(19) United States
(12) Patent Application Publication
(10) Pub. No.: US 2025/0305061 A1
(43) Pub. Date: Oct. 2, 2025

(54) METHODS AND SYSTEMS FOR INFERRING GENE EXPRESSION USING CELL-FREE DNA FRAGMENTS

(71) Applicant: Freenome Holdings, Inc., South San Francisco, CA (US)

(72) Inventors: Emily LEFF, Pacifica, CA (US); Alexander TSENG, Mountain View, CA (US); Victoria CHEUNG, Menlo Park, CA (US); Rebecca GUPTE, Foster City, CA (US); Ehsan TABARI, San Francisco, CA (US); Francesco VALLANIA, San Francisco, CA (US); Hayley DONNELLA, Austin, TX (US); John ST. JOHN, Brisbane, CA (US); Alexander LOVEJOY, Cottage Grove, OR (US); Peter ULZ, Graz (AT)

(21) Appl. No.: 19/092,296

(22) Filed: Mar. 27, 2025

Related U.S. Application Data
(63) Continuation of application No. PCT/US2025/021646, filed on Mar. 26, 2025.
(60) Provisional application No. 63/570,508, filed on Mar. 27, 2024.

(51) Int. Cl. C12Q 1/6886; C12Q 1/6874

(52) U.S. Cl. CPC C12Q 1/6886; C12Q 1/6874; C12Q 2600/154

(57) ABSTRACT

Methods and systems disclosed herein can improve inference of gene expression using cell-free DNA fragments. In an aspect, the present disclosure provides a computer-implemented method for inferring gene expression, the method comprising: obtaining a biological sample from a subject; extracting cell-free deoxyribonucleic acid (cfDNA) from the biological sample, wherein the cfDNA comprises a plurality of cfDNA fragments; performing a sequencing assay on the plurality of cfDNA fragments to generate a plurality of cfDNA sequencing fragments; computer processing the plurality of cfDNA sequencing fragments; and calculating, based at least in part on the computer processing, a gene expression score for a gene in a plurality of genes, wherein the gene expression score indicates a probability of expression or non-expression of the gene in the plurality of genes.

### CROSS-REFERENCE

This application is a continuation of International Application No. PCT/US2025/021646, filed Mar. 26, 2025, which claims the benefit of U.S. Provisional Application No. 63/570,508, filed Mar. 27, 2024, each of which is incorporated by reference herein in its entirety.

### BACKGROUND

Cell-free DNA (cfDNA) circulating in blood plasma may arise primarily from cellular chromatin fragmentation and release due to cell death. The assessment of fragmentomic (e.g., fragment length) features of cfDNA may enable gene expression inference and tissue-of-origin classification with potential applications for noninvasive cancer detection. However, due to low depth of coverage of sites of interest, current whole genome sequencing (WGS) methods may not be capable of inferring expression of individual genes or limited gene sets.

### SUMMARY

Aspects disclosed herein provide methods for preparing a methylation sequencing library for inferring gene expression, the method comprising: (a) obtaining a biological sample from a subject; (b) extracting cell-free deoxyribonucleic acid (cfDNA) from the biological sample, wherein the cfDNA comprises a plurality of cfDNA fragments; (c) providing conditions capable of converting unmethylated cytosines to uracils in the cfDNA fragments to produce a plurality of converted cfDNA fragments; (d) enriching the plurality of converted cfDNA fragments to produce enriched converted cfDNA fragment molecules, wherein the enriching comprises contacting the plurality of converted cfDNA fragments with a probe set comprising hybridization probes having sequence complementarity to at least two transcription start site (TSS) sequences of a plurality of TSS sequences selected from the genes listed in Tables 1-5; (e) amplifying the enriched converted cfDNA fragment molecules to produce amplified enriched converted cfDNA fragments; and (f) determining a nucleic acid sequence of a plurality of cfDNA sequencing fragments of the amplified enriched converted cfDNA fragments. In some embodiments, the method further comprises processing the plurality of cfDNA sequencing fragments, wherein the processing comprises calculating a gene expression score for one or more genes of a plurality of genes, wherein the gene expression score indicates a probability of expression or non-expression of the one or more genes of the plurality of genes. In some embodiments, the plurality of TSS sequences are selected from the genes listed in Table 1. In some embodiments, the plurality of TSS sequences are selected from the genes listed in Table 2. In some embodiments, the plurality of TSS sequences are selected from the genes listed in Table 3. In some embodiments, the plurality of TSS sequences are selected from the genes listed in Table 4. In some embodiments, the plurality of TSS sequences are selected from the genes listed in Table 5. In some embodiments, the biological sample comprises a blood sample or a cellular sample. In some embodiments, the blood sample comprises a plasma sample, a serum sample, or a buffy coat sample. In some embodiments, the cellular sample comprises a tissue sample, a biopsy sample, or a plurality of cells from a cell line. In some embodiments, deoxyribonucleic acid (DNA) from the biological sample is treated with one or more nucleases prior to (b). In some embodiments, the one or more nucleases comprises micrococcal nuclease (MNase). In some embodiments, the method further comprises performing a sequencing assay on the plurality of cfDNA fragments. In some embodiments, the sequencing assay comprises next generation sequencing (NGS). In some embodiments, the NGS comprises whole genome sequencing (WGS) or targeted sequencing. In some embodiments, the sequencing assay comprises bisulfite conversion, enzymatic conversion, or TET-assisted pyridine borane sequencing (TAPS) conversion. In some embodiments, the method further comprises determining fragmentation patterns in the plurality of cfDNA sequencing fragments. In some embodiments, the method further comprises using the fragmentation patterns to train a machine learning classifier capable of distinguishing between gene expression and gene non-expression. In some embodiments, the gene expression score comprises a value of between 0 and 1. In some embodiments, a gene expression score of 0 corresponds to non-expression of the gene. In some embodiments, a gene expression score of 1 corresponds to expression of the gene. In some embodiments, the one or more genes comprise epithelial cell-related genes. In some embodiments, the one or more genes comprise a member selected from the group consisting of: SOWAHB, TMEM63C, SOX2, TMEM184A, NBL1, B4GALNT2, TFAP2B, RND2, TP63, ATG9B, IGSF9, TMEM82, C10orf99, LOXL1, and GRB7. In some embodiments, the one or more genes comprise transcriptional targets. In some embodiments, the transcriptional targets comprise a member selected from the group consisting of: SOWAHB, TMEM63C, SOX2, TMEM184A, NBL1, B4GALNT2, TFAP2B, RND2, TP63, ATG9B, IGSF9, TMEM82, C10orf99, LOXL1, and GRB7. In some embodiments, the method further comprises detecting the expression or the non-expression of the one or more genes with an accuracy of at least 70%, at least 80%, or at least 90%. In some embodiments, the method further comprises detecting the expression or the non-expression of the one or more genes with a sensitivity of at least 70%, at least 80%, or at least 90%. In some embodiments, the method further comprises detecting the expression or the non-expression of the one or more genes with a specificity at least 70%, at least 80%, or at least 90%. In some embodiments, the subject is a human. In some embodiments, the gene expression score is used to distinguish between a diseased biological sample and a healthy biological sample. In some embodiments, the diseased biological sample is a sample obtained or derived from a subject having cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the method further comprises detecting a presence or an absence of a disease in the subject based at least in part on the gene expression score of the one or more genes. In some embodiments, the method further comprises minimal residual disease monitoring. In some embodiments, the disease comprises cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the cancer is stage I cancer, stage II cancer, stage III cancer, or stage IV cancer. In some embodiments, the method further comprises administering a treatment to the subject based on detecting the presence of the disease in the subject. In some embodiments, the method further comprises administering a treatment to the subject based on determining a cancer subtype of the subject, or based on inferred gene expression patterns of the cancer or subject.

Aspects disclosed herein provide methods for preparing a methylation sequencing library for inferring gene expression, the method comprising: (a) obtaining a biological sample from a subject; (b) extracting cell-free deoxyribonucleic acid (cfDNA) from the biological sample, wherein the cfDNA comprises a plurality of cfDNA fragments; (c) providing conditions capable of converting unmethylated cytosines to uracils in the cfDNA fragments to produce a plurality of converted cfDNA fragment molecules; (d) amplifying the plurality of converted cfDNA fragment molecules to produce amplified converted cfDNA fragments; (e) determining a nucleic acid sequence of a plurality of cfDNA sequencing fragments of the amplified converted cfDNA fragments; and (f) processing the plurality of cfDNA sequencing fragments, wherein the processing comprises calculating a gene expression score for one or more genes in a plurality of genes, wherein the gene expression score indicates a probability of expression or non-expression of the one or more genes of the plurality of genes. In some embodiments, the biological sample comprises a blood sample or cellular sample. In some embodiments, the blood sample comprises a plasma sample, a serum sample, or a buffy coat sample. In some embodiments, the cellular sample comprises a tissue sample, a biopsy sample, or a plurality of cells from a cell line. In some embodiments, deoxyribonucleic acid (DNA) from the biological sample is treated with one or more nucleases prior to (b). In some embodiments, the one or more nucleases comprises micrococcal nuclease (MNase). In some embodiments, the method further comprises performing a sequencing assay on the plurality of cfDNA fragments. In some embodiments, the sequencing assay comprises next generation sequencing (NGS). In some embodiments, the NGS comprises whole genome sequencing (WGS) or targeted sequencing. In some embodiments, the sequencing assay comprises bisulfite conversion, enzymatic conversion, or TET-assisted pyridine borane sequencing (TAPS) conversion. In some embodiments, the method further comprises determining fragmentation patterns in the plurality of cfDNA sequencing fragments. In some embodiments, the method further comprises using the fragmentation patterns to train a machine learning classifier capable of distinguishing between gene expression and gene non-expression. In some embodiments, the gene expression score comprises a value of between 0 and 1. In some embodiments, a gene expression score of 0 corresponds to non-expression of the gene. In some embodiments, a gene expression score of 1 corresponds to expression of the gene. In some embodiments, the method further comprises detecting the expression or the non-expression of the one or more genes with an accuracy of at least 70%, at least 80%, or at least 90%. In some embodiments, the method further comprises detecting the expression or the non-expression of the one or more genes with a sensitivity of at least 70%, at least 80%, or at least 90%. In some embodiments, the method further comprises detecting the expression or the non-expression of the one or more genes with a specificity of at least 70%, at least 80%, or at least 90%. In some embodiments, the subject is a human. In some embodiments, the gene expression score is used to distinguish between a diseased biological sample and healthy biological sample. In some embodiments, the diseased biological sample is a biological sample obtained or derived from a subject having cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the method further comprises detecting a presence or an absence of a disease in the subject based at least in part on the gene expression score of the one or more genes. In some embodiments, the method further comprises minimal residual disease monitoring. In some embodiments, the disease comprises cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the cancer is stage I cancer, stage II cancer, stage III cancer, or stage IV cancer. In some embodiments, the method further comprises administering a treatment to the subject based on detecting the presence of the disease in the subject. In some embodiments, the method further comprises administering a treatment to the subject based on determining a cancer subtype of the subject, or based on inferred gene expression patterns of the cancer or subject.

Aspects disclosed herein provide methods for preparing a sequencing library for inferring gene expression, the method comprising: (a) obtaining a biological sample from a subject; (b) extracting cell-free deoxyribonucleic acid (cfDNA) from the biological sample, wherein the cfDNA comprises a plurality of cfDNA fragments; (c) enriching the plurality of cfDNA fragments to produce enriched cfDNA fragment molecules, wherein the enriching comprises contacting the plurality of cfDNA fragments with a probe set comprising hybridization probes having sequence complementarity to at least two transcription start site (TSS) sequences of a plurality of TSS selected from the genes listed in Tables 1-5; (d) amplifying the enriched cfDNA fragment molecules to produce amplified enriched cfDNA fragments; (e) determining a nucleic acid sequence of a plurality of cfDNA sequencing fragments of the amplified enriched cfDNA fragments; and (f) processing the plurality of sequenced enriched cfDNA fragments, wherein the processing comprises calculating a gene expression score for one or more genes of a plurality of genes, wherein the gene expression score indicates a probability of expression or non-expression of the one or more genes of the plurality of genes. In some embodiments, the plurality of TSS sequences are selected from the genes listed in Table 1. In some embodiments, the plurality of TSS sequences are selected from the genes listed in Table 2. In some embodiments, the plurality of TSS sequences are selected from the genes listed in Table 3. In some embodiments, the plurality of TSS sequences are selected from the genes listed in Table 4. In some embodiments, the plurality of TSS sequences are selected from the genes listed in Table 5. In some embodiments, the biological sample comprises a blood sample or cellular sample. In some embodiments, the blood sample comprises a plasma sample, a serum sample, or a buffy coat sample. In some embodiments, the cellular sample comprises a tissue sample, a biopsy sample, or a plurality of cells from a cell line. In some embodiments, deoxyribonucleic acid (DNA) from the biological sample is treated with one or more nucleases prior to (b). In some embodiments, the one or more nucleases comprise MNase. In some embodiments, the method further comprises performing a sequencing assay on the plurality of cfDNA fragments. In some embodiments, the sequencing assay comprises next generation sequencing (NGS). In some embodiments, the NGS comprises whole genome sequencing (WGS) or targeted sequencing. In some embodiments, the sequencing assay comprises bisulfite conversion, enzymatic conversion, or TET-assisted pyridine borane sequencing (TAPS) conversion. In some embodiments, the method further comprises determining fragmentation patterns in the plurality of cfDNA sequencing fragments. In some embodiments, the method further comprises using the fragmentation patterns to train a machine learning classifier capable of distinguishing between gene expression and gene non-expression. In some embodiments, the gene expression score comprises a value of between 0 and 1. In some embodiments, a gene expression score of 0 corresponds to non-expression of the gene. In some embodiments, a gene expression score of 1 corresponds to expression of the gene. In some embodiments, the method further comprises detecting the expression or the non-expression of the one or more genes with an accuracy of at least 70%, at least 80%, or at least 90%. In some embodiments, the method further comprises detecting the expression or the non-expression of the one or more genes with a sensitivity of at least 70%, at least 80%, or at least 90%. In some embodiments, the method further comprises detecting the expression or the non-expression of the one or more genes with a specificity of at least 70%, at least 80%, or at least 90%. In some embodiments, the subject is a human. In some embodiments, the gene expression score is used to distinguish between a diseased biological sample and healthy biological sample. In some embodiments, the diseased biological sample is a biological sample obtained or derived from a subject having cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the method further comprises detecting a presence or an absence of a disease in the subject based at least in part on the gene expression score of the one or more genes. In some embodiments, the method further comprises minimal residual disease monitoring. In some embodiments, the disease comprises cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the cancer is stage I cancer, stage II cancer, stage III cancer, or stage IV cancer. In some embodiments, the method further comprises administering a treatment to the subject based on detecting the presence of the disease in the subject. In some embodiments, the method further comprises administering a treatment to the subject based on determining a cancer subtype of the subject, or based on inferred gene expression patterns of the cancer or subject.

Aspects disclosed herein provide methods for preparing a sequencing library for inferring gene expression, the method comprising: (a) obtaining a biological sample from a subject; (b) extracting cell-free deoxyribonucleic acid (cfDNA) from the biological sample, wherein the cfDNA comprises a plurality of cfDNA fragments; (c) amplifying the plurality of cfDNA fragments to produce amplified cfDNA fragments; (d) determining a nucleic acid sequence of a plurality of cfDNA sequencing fragments of the amplified cfDNA fragments; and (e) processing the plurality of sequenced cfDNA fragments, wherein the processing comprises calculating a gene expression score of one or more genes of a plurality of genes, wherein the gene expression score indicates a probability of expression or non-expression of the one or more genes of the plurality of genes. In some embodiments, the biological sample comprises a blood sample or cellular sample. In some embodiments, the blood sample comprises a plasma sample, a serum sample, or a buffy coat sample. In some embodiments, the cellular sample comprises a tissue sample, a biopsy sample, or a plurality of cells from a cell line. In some embodiments, deoxyribonucleic acid (DNA) from the biological sample is treated with one or more nucleases prior to (b). In some embodiments, the one or more nucleases comprise MNase. In some embodiments, the method further comprises performing a sequencing assay on the plurality of cfDNA fragments. In some embodiments, the sequencing assay comprises next generation sequencing (NGS). In some embodiments, the NGS comprises whole genome sequencing (WGS) or targeted sequencing. In some embodiments, the sequencing assay comprises bisulfite conversion, enzymatic conversion, or TET-assisted pyridine borane sequencing (TAPS) conversion. In some embodiments, the method further comprises determining fragmentation patterns in the plurality of cfDNA sequencing fragments. In some embodiments, the method further comprises using the fragmentation patterns to train a machine learning classifier capable of distinguishing between gene expression and gene non-expression. In some embodiments, the gene expression score comprises a value of between 0 and 1. In some embodiments, a gene expression score of 0 corresponds to non-expression of the gene. In some embodiments, a gene expression score of 1 corresponds to expression of the gene. In some embodiments, the method further comprises detecting the expression or the non-expression of the one or more genes with an accuracy of at least 70%, at least 80%, or at least 90%. In some embodiments, the method further comprises detecting the expression or the non-expression of the one or more genes with a specificity of at least 70%, at least 80%, or at least 90%. In some embodiments, the method further comprises detecting the expression or the non-expression of the one or more genes with a sensitivity of at least 70%, at least 80%, or at least 90%. In some embodiments, the subject is a human. In some embodiments, the gene expression score is used to distinguish between a diseased biological sample and a healthy biological sample. In some embodiments, the diseased biological sample is a biological sample obtained or derived from a subject having cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the method further comprises detecting a presence or an absence of a disease in the subject based at least in part on the gene expression score of the one or more genes. In some embodiments, the method further comprises minimal residual disease monitoring. In some embodiments, the disease comprises cancer. In some embodiments, the cancer is selected from the group consisting of breast cancer, diffuse large B cell lymphoma, lung cancer, pancreatic cancer, liver cancer, colorectal cancer, gastric cancer, prostate cancer, ovarian cancer, and bile duct cancer. In some embodiments, the cancer is stage I cancer, stage II cancer, stage III cancer, or stage IV cancer. In some embodiments, the method further comprises administering a treatment to the subject based on detecting the presence of the disease in the subject. In some embodiments, the method further comprises administering a treatment to the subject based on determining a cancer subtype of the subject, or based on inferred gene expression patterns of the cancer or subject.

Aspects disclosed herein provide non-transitory computer-readable memory storing one or more instructions executable by one or more processors, that when executed by the one or more processors cause the one or more processors to perform processing, comprising: (a) obtaining a biological sample from a subject; (b) extracting cell-free DNA (cfDNA) from the biological sample, wherein the cfDNA comprises a plurality of cfDNA fragments; (c) performing a sequencing assay on the plurality of cfDNA fragments to generate a plurality of cfDNA sequencing fragments; (d) computer processing the plurality of cfDNA sequencing fragments; and (e) calculating, based at least in part on the computer processing, a gene expression score for one or more genes of a plurality of genes, wherein the gene expression score indicates a probability of expression or non-expression of the one or more genes of the plurality of genes.

Aspects disclosed herein provide computer systems for inferring gene expression, the system comprising: (a) a non-transitory memory; and (b) a processor in communication with the non-transitory memory, the processor configured to execute the following operations in order to effectuate a method comprising the operations of: (i) obtaining a biological sample from a subject; (ii) extracting cell-free DNA (cfDNA) from the biological sample, wherein the cfDNA comprises a plurality of cfDNA fragments; (iii) performing a sequencing assay on the plurality of cfDNA fragments to generate a plurality of cfDNA sequencing fragments; (iv) computer processing the plurality of cfDNA sequencing fragments; and (v) calculating, based at least in part on the computer processing, a gene expression score for one or more genes of a plurality of genes, wherein the gene expression score indicates a probability of expression or non-expression of the one or more genes of the plurality of genes.

Additional aspects and advantages of the present disclosure will become readily apparent to those skilled in this art from the following detailed description, wherein only illustrative examples of the present disclosure are shown and disclosed. As will be realized, the present disclosure is capable of other and different examples, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the invention are set forth with particularity in the appended claims. A better understanding of the features and advantages of the present methods and systems will be obtained by reference to the following detailed description that sets forth illustrative examples, in which the principles of the methods and systems are utilized, and the accompanying drawings (also “figure” and “FIG.” herein), of which:

FIG. 1 shows a computer system that is programmed or otherwise configured to perform methods of the present disclosure.

FIG. 2 shows micrococcal nuclease (MNase)-digested blends of 100% cell lines of epithelial colorectal cancer cell line (LS180), T cells (CD4), and monocytes (CD14).

FIG. 3 shows a flow chart for an embodiment of the entire workflow process of generating TSS-GAP scores, which includes dividing the 19,910 protein-coding genes into training and test sets, denoising V-plots, and using the training genes to train a model to classify genes as “on” or “off” for prediction on the test (holdout) genes, providing the computational basis for calculating TSS-GAP scores.

FIG. 4 shows a flow chart for obtaining the limit of detection (LoD) by comparing associated TSS-GAP scores between MNase-digested colorectal cancer cell line (LS180) DNA and healthy donor-derived DNA. The number of genes is cumulative across concentrations of LS180. Detectability is determined if the minimum TSS-GAP score for a gene at a concentration of LS180 is greater than the mean TSS-GAP score for the same gene among four healthy PBMCs.

FIG. 5 shows several V-plots for the FOXA1 and MUC6 genes, where the x-axis represents distance to TSS in base pairs and the y-axis represents fragment length in base pairs.

FIG. 6 shows several box plots with TSS-GAP scores for 15 genes, where the x-axis represents one of the 15 genes, and the y-axis represents the TSS-GAP score.

FIGS. 7A-7B show bar graphs depicting the top enriched gene pathways and PaGenBase gene profiles in 0.1% LS180 and all concentrations of LS180 ranked most enriched to least enriched by-log (p-adjusted).

FIG. 8 illustrates that CRC epithelial signatures can be detected at low level for TSS-GAP. TSS-GAP is able to detect epithelial CRC-associated pathways and gene expression profiles at all concentrations of LS180 (epithelial cell differentiation, colon tissue/epithelial cell signatures). TSS-GAP can also detect the same signal at significantly lower concentrations of LS180 (cell-cell adhesion, regulation of epithelial cell proliferation, colorectal adenocarcinoma tissue signatures and epithelial cell signatures at 0.1%.

FIG. 9A illustrates an example of a workflow used in the methods and systems described herein where DNA (e.g., cell-free DNA, MNase-treated DNA, fragmented DNA, etc.) is used to generate a sequencing library (library prep). An enzymatic conversion operation is performed (CpG conversion) and hybrid capture panels comprising regions flanking the transcription start site (TSS) of a panel of genes are provided and next generation sequencing is performed to generate reads for TSS-GAP and methylation. Computational analysis of the NGS methylation sequencing reads for TSS-GAP is then performed to generate TSS-GAP scores.

FIG. 9B shows an example of a workflow used in the methods and systems described herein where DNA (e.g., cell-free DNA, MNase-treated DNA, fragmented DNA, etc.) is used to generate a sequencing library (library prep). NGS at a coverage of about 30×is performed to generate reads for TSS-GAP, transcription factor binding accessibility (TFBA), or a combination thereof. Computational analysis of the whole genome sequencing reads for TSS-GAP is then performed to generate TSS-GAP scores.

FIG. 10 shows an image illustrating data quality control and data preprocessing for TEM-seq and whole genome sequencing (WGS).

FIG. 11 shows several V-plots for POMGNT1, UROD, LRRC8C, BCAN, LRRC71, and HSD3B1 in MNase-Digested (MN-D) PBMCs and in cfDNA.

FIG. 12A shows an example of a workflow used in the methods and systems described herein.

FIG. 12B shows an example of ideal DNA fragment profiles associated with cfDNA, MNase-digestion, and ATAC-seq around an active transcription start site.

FIG. 13A shows an example of a graph where cfDNA TSS-GAP score represents the x-axis and MN-D PBMCs TSS-GAP score represents the y-axis.

FIG. 13B shows an example of a graph where cfDNA TSS-GAP score represents the x-axis and MN-D PBMCs TSS-GAP score represents the y-axis.

FIG. 13C shows an example of a graph where cfDNA TSS-GAP score represents the x-axis and MN-D PBMCs TSS-GAP score represents the y-axis.

FIG. 13D shows an example of a graph where cfDNA TSS-GAP score represents the x-axis and MN-D PBMCs TSS-GAP score represents the y-axis.

FIG. 14 shows an example of a schematic diagram comparing dataset-level modeling and sample-specific modeling approaches. In dataset-level modeling, training gene subsets from all samples are combined into a single large training set to generate a unified dataset-level model. This model is then used to predict holdout genes, producing a consolidated results matrix. Alternatively, in sample-specific modeling, each sample is treated independently, training its own model using its training gene subset. Each trained model then predicts holdout genes specific to the individual sample, resulting in sample-specific results matrices.

FIG. 15A shows an example of a schematic diagram illustrating a first approach for generating TSS-GAP scores wherein the modeling frameworks that are specific to each dataset, training with only the healthy subset of samples. In this embodiment, datasets A and B are treated as discrete entities, with models trained separately within each dataset. Each dataset is first divided into healthy and cases subsets. Pre-defined training genes from the healthy subset are used to train models, which then predict holdout genes from both subsets. This framework supports both dataset-level modeling (DSL), where a single model is trained per dataset, and sample-specific modeling (SS), where individual models are trained per sample. If multiple models are generated per sample, an ensemble approach is applied as necessary.

FIG. 15B shows an example of a schematic diagram illustrating a second approach for generating TSS-GAP scores wherein the modeling frameworks that are specific to each dataset, training without subgroup separation. In this embodiment, datasets A and B are treated as discrete entities, with models trained separately within each dataset. All samples within a dataset contribute to model training using a predefined set of training genes. The trained models are then used to predict holdout genes for the same dataset, generating a results matrix of TSS-GAP scores. This framework supports both dataset-level modeling (DSL), where a single model is trained per dataset, and sample-specific modeling (SS), where individual models are trained per sample. If multiple models are generated per sample, an ensemble approach is applied as necessary.

FIG. 15C shows an example of a schematic diagram illustrating a third approach for generating TSS-GAP scores wherein cross-dataset modeling occurs with subgroup-based training. In this embodiment, datasets A and B are treated as discrete entities, with dataset A used to train a master model. Within dataset A, only the healthy subset is used for training with the predefined set of training genes. The trained master model is then applied to predict on holdout genes in other datasets, including healthy and cases subsets. This framework supports both dataset-level modeling (DSL), where a single model is trained per dataset, and sample-specific modeling (SS), where individual models are trained per sample. If multiple models are generated per sample, an ensemble approach is applied as necessary.

FIG. 15D shows an example of a schematic diagram illustrating a third approach for generating TSS-GAP scores wherein cross-dataset modeling occurs without subgroup separation. In this embodiment, datasets A and B are treated as discrete entities, with dataset A used to train a master model. Within dataset A, all samples are used for training with the predefined set of training genes. The trained master model is then applied to predict on holdout genes in other datasets, including healthy and cases subsets. This framework supports both dataset-level modeling (DSL), where a single model is trained per dataset, and sample-specific modeling (SS), where individual models are trained per sample. If multiple models are generated per sample, an ensemble approach is applied as necessary.

### DETAILED DESCRIPTION

While various embodiments of the invention have been shown and disclosed herein, it will be obvious to those skilled in the art that such embodiments are provided by way of example only. Numerous variations, changes, and substitutions may occur to those skilled in the art without departing from the invention. It should be understood that various alternatives to the embodiments of the invention disclosed herein may be employed.

Where values are disclosed as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “plasma cell-free DNA”, “circulating free DNA” or “cell-free DNA” (cfDNA) generally refers to deoxyribonucleic acid (DNA) that was first detected in human blood plasma in 1948. (Mandel, P. Metais, P., C R Acad. Sci. Paris, 142, 241-243 (1948), which is incorporated by reference herein in its entirety). Much of the circulating nucleic acids in blood may arise from necrotic or apoptotic cells (Giacona, M. B., et al., Pancreas, 17, 89-97 (1998), which is incorporated by reference herein in its entirety) and greatly elevated levels of nucleic acids from apoptosis is observed in diseases such as cancer. (Giacona, M. B., et al., Pancreas, 17, 89-97 (1998); Fournie, G. J., et al., Cancer Lett, 91, 221-227 (1995), which is incorporated by reference herein in its entirety). In cancer, circulating DNA bears hallmark signs of the disease, including mutations in oncogenes and microsatellite alterations. These circulating DNA may be referred to as circulating tumor DNA (ctDNA). Viral genomic sequences, DNA, or RNA in plasma is a potential biomarker for disease.

The term “cell-free fraction” of a biological sample, as used herein, generally refers to a fraction of the biological sample that is substantially free of cells. The cell-free fraction may be blood serum or blood plasma. In some embodiments, the cell-free fraction of blood is preferably blood serum or blood plasma. As used herein, the term “substantially free of cells” may refer to a preparation from the biological sample comprising fewer than about 20,000 cells per ml, fewer than about 2,000 cells per ml, fewer than about 200 cells per ml, or fewer than about 20 cells per ml.

As used herein, the term “substantially free of cells” generally refers to a preparation from the biological sample comprising fewer than about 20,000 cells per mL, fewer than about 2,000 cells per mL, fewer than about 200 cells per mL, or fewer than about 20 cells per mL. Genomic DNA (gDNA) refers to non-fragmented DNA that is released from white blood cells contaminating the blood cell-free fraction. To mitigate gDNA from contaminating samples, a highly controlled sample processing workflow may be implemented, and specimens may be screened against the presence of gDNA. Genomic DNA may not be excluded from the acellular sample and may comprise from about 0% to about 90% of the nucleic acids that are present in the sample.

As used herein, the term “nucleic acid” generally refers to a polynucleotide comprising two or more nucleotides. It may be DNA or RNA. The nucleic acid may be a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, identity to the nucleotide sequence of the original nucleic acid.

As used herein, the term “methylation conversion methods” or “methylation enrichment methods” or “methylation conversion agents” refers to a method in which a nucleic acid molecule is subjected to conditions sufficient to convert unmethylated cytosines in the nucleic acid molecule to uracils. The methods are useful for differentiating methylated cytosines from unmethylated cytosines in a nucleic acid molecule. Methylation conversion methods or methylation conversion agents can include bisulfite conversion or bisulfite sequencing may be used for DNA methylation analysis. Bisulfite sequencing is a convenient and effective method of mapping DNA methylation to individual bases. Additionally, methylation conversion methods or methylation conversion agents can include enzymatic methylation (EM) conversion. Enzymatic methylation conversion is mediated by non-destructive enzymatic reactions involving a ten-eleven translocation (TET) enzyme and a cytosine-deaminating enzyme (e.g., APOBEC) to convert unmethylated (but not methylated) cytosines to uracils. Other embodiments such as Tet-assisted pyridine borane sequencing (TAPS) combine enzymatic reactions such as TET together with chemical treatments (e.g., pyridine borane).

As used herein, the term “enzymatic methylation” or “enzymatic methyl” or “EM conversion” or “EM-seq” refers to a method in which a nucleic acid molecule is subjected to conditions sufficient to convert unmethylated cytosines in the nucleic acid molecule to uracils by treatment with one or more enzymes. In some cases, the method does not comprise treatment with bisulfite (e.g., chemical treatment).

As used herein, the term “methylcytosine dioxygenase”, “dioxygenase”, or “oxygenase” refers to an enzyme that converts 5mC to 5hmC. Non-limiting examples of methylcytosine dioxygenases include, e.g., ten eleven translocation (TET) enzymes, e.g., TET1, TET2, TET3, Naeglaria TET, and genetically engineered versions and/or variants thereof. TET2 is an example of a methylcytosine dioxygenase that oxidizes at least 90%, at least 92%, at least 94%, at least 96%, at least 98%, or at least 99% of all 5mC.

As used herein, the term “cytidine deaminase” refers to an enzyme that deaminates cytosine (C) to form uracil (U). Non-limiting examples of cytidine deaminases include the apolipoprotein B mRNA-editing enzyme, catalytic polypeptide (APOBEC) family of cytidine deaminases, such as APOBEC3A. In any embodiment, a cytidine deaminase described herein may have an amino acid sequence that is at least 90% identical to (e.g., at least 95% identical to) the amino acid sequence of GenBank accession number AKE33285.1, which is the sequence of human APOBEC3A. In some embodiments, a cytidine deaminase described herein converts unmodified cytosine to uracil with an efficiency of at least 95%, 98% or 99%, preferably at least 99%.

As used herein, the term “glucosyltransferase” or “GT” refers to an enzyme that catalyzes the transfer of a beta-D-glucosyl or alpha-D-glucosyl residue from UDP-glucose to 5hmC residue to form 5ghmC. APOBEC can convert 5hmC to U at a low rate relative to converting C or 5mC to U. An example of a GT is T4-betaGT (BGT). In one example, GT may be used concurrently with a dioxygenase. This combination ensures that deamination of 5hmC is blocked such that less than 5%, less than 3%, or less than 1% of 5hmC is converted to U by the deaminase. In another example, GT may be used together with dioxygenase in the same reaction mix with DNA such that the dioxygenase converts 5mC to 5hmC and 5caC, and the GT converts any residual 5hmC to 5ghmC to ensure only cytosine is deaminated.

The term “Next Generation Sequencing” or “NGS” generally applies to sequencing libraries of genomic fragments of a size of less than 1 kb.

As used herein, the term “subject” generally refers to an individual, entity or a medium that has or is suspected of having testable or detectable genetic information or material. A subject can be a person, individual, or patient. The subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a cancer or a stage of a cancer of the subject. As an alternative, the subject can be asymptomatic with respect to such health or physiological state or condition.

As used herein, the term “sample” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free protein and/or cell-free polypeptides. A biological sample may be tissue (e.g., tissue obtained by biopsy), blood (e.g., whole blood), plasma, serum, sweat, urine, saliva, or a derivative thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops), a tumor sample, a tissue sample, a urine sample, or a cell (e.g., tissue) sample.

### I. Method for Inferring Gene Expression

In an aspect, the present disclosure provides methods for preparing a sequencing library for inferring gene expression. The sequencing library may be a methylation sequencing library. The methods may comprise obtaining a biological sample from a subject. The methods may comprise extracting cell-free deoxyribonucleic acid (cfDNA) from the biological sample, wherein the cfDNA comprises a plurality of cfDNA fragments. The methods may comprise providing conditions capable of converting unmethylated cytosines to uracils in the cfDNA fragments to produce a plurality of converted cfDNA fragments. The methods may comprise enriching the plurality of converted or unconverted cfDNA fragments to produce enriched converted or unconverted cfDNA fragment molecule. The enriching may comprise contacting the plurality of converted or unconverted cfDNA fragments with a probe set comprising hybridization probes having sequence complementarity to at least two transcription start site (TSS) sequences of a plurality of TSS sequences selected from the genes listed in Tables 1-5. The methods may comprise amplifying the enriched converted or unconverted cfDNA fragment molecules to produce amplified enriched converted or unconverted cfDNA fragments. The methods may comprise determining a nucleic acid sequence of a plurality of cfDNA sequencing fragments of the amplified enriched converted or unconverted cfDNA fragments. The methods may comprise processing the plurality of cfDNA sequencing fragments. The processing may comprise calculating a gene expression score for one or more genes of a plurality of genes. The gene expression score may indicate a probability of expression or non-expression of the one or more genes of the plurality of genes. The methods may comprise detecting a presence or an absence of a disease in the subject based on the determining a nucleic acid sequence of a plurality of cfDNA sequencing fragments. The methods may comprise detecting a presence or an absence of a disease in the subject based on the processing the plurality of cfDNA sequencing fragments. The methods may comprise detecting a presence or an absence of a disease in the subject based on the calculated gene expression score for one or more genes of a plurality of genes.

In certain embodiments, the extracted cfDNA may comprise a plurality of cfDNA fragments. The method may include performing a sequencing assay on the plurality of cfDNA fragments to generate a plurality of cfDNA sequencing fragments. The method may include computer processing the plurality of cfDNA sequencing fragments. The method may include calculating a gene expression score for a gene in a plurality of genes. The gene expression score may indicate a probability of expression or non-expression of the gene in the plurality of genes. The calculation may be based in part on the computer processing.

In other embodiments, the extracted DNA may undergo enzymatic processing to generate a plurality of DNA fragments. The method may include performing a sequencing assay on the plurality of DNA fragments to generate a plurality of DNA sequencing fragments. The method may include computer processing the plurality of DNA sequencing fragments. The method may include calculating a gene expression score for a gene in a plurality of genes. The gene expression score may indicate a probability of expression or non-expression of the gene in the plurality of genes. The calculation may be based in part on the computer processing.

The biological sample may be cell-free. The biological sample may comprise nucleic acids, such as DNA or RNA. The DNA may be cell-free DNA. The RNA may be cell-free RNA, such as cell-free mRNA.

The biological sample may comprise a blood sample. The blood sample may be a plasma sample. The blood sample may be a serum sample. The blood sample may be a buffy coat sample.

The biological sample may comprise a cellular source. The cellular source may comprise a tissue sample. The cellular source may comprise a biopsy sample. The cellular source may comprise one or more cells isolated from a cell line.

The method may include enzymatic processing of the extracted DNA from a biological sample comprising a cellular source. The enzymatic processing of the extracted DNA may comprise treatment with one or more nucleases. In certain embodiments, the enzymatic treatment with one or more nucleases reflects the underlying nucleosome positioning of the extracted DNA.

The method may include extracting cfDNA from the biological sample. The cfDNA may comprise a plurality of cfDNA fragments. In some cases, the plurality of cfDNA fragments may comprise more than or equal to 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 9,000, 10,00, 25,000, 50,000, or 100,000 cfDNA fragments. The cfDNA fragments may be various lengths (base pairs). In some cases, the cfDNA fragments have a length of more than or equal to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150,160, 170, 180, 190, 200, 210, 220, 230, 240, 250 260, 270, 280, 290, 300, 310, 320, 330, 340, or 350 base pairs. Each cfDNA fragment in the plurality of cfDNA fragments may comprise the same or different lengths in base pairs.

The method may further include library preparation methods including, but not limited to, end-repair, A-tailing, adapter ligation, or any other preparation performed on the cfDNA fragments to permit subsequent sequencing of DNA. In certain examples, a prepared cell-free nucleic acid library sequence can contain adapters, sequence tags, index barcodes or combinations thereof that are ligated onto cell-free nucleic acid sample molecules. Various commercially available kits are available to facilitate library preparation for NGS approaches. Advances and the development of various library preparation technologies have expanded the application of NGS to fields such as epigenetics.

The method may also include hybrid capture being carried out on the prepared library sequences using specific probes. In some embodiments, the term “specific probe”, as used herein, generally refers to a probe that is specific for a region. In some embodiments, the specific probes are designed based on using the human genome as a reference sequence and using specific genomic regions of interest. Therefore, when carrying out the hybrid capture by using the specific probes of some embodiments, the sequences in the sample genome which are complementary to the target sequences may be captured efficiently.

The method may also include methyl conversion to convert the DNA for methylation sequencing. In such an embodiment, DNA methylation analysis may be coupled with sequencing to determine whether a portion of cfDNA is likely to be pre-cancerous or tumor-derived. DNA methylation is a covalent modification of DNA and a stable inherited mark that can play an important role in repressing gene expression and regulating chromatin architecture. In humans, DNA methylation primarily occurs at cytosine residues in CpG dinucleotides. Unlike other dinucleotides, CpGs are not evenly distributed across the genome and can be concentrated in short CpG-rich DNA regions called CpG islands. In general, the majority of the CpG sites in the genome are ˜70-75% methylated. However, methylation patterns differ from cell type to cell type, reflecting their role in regulating cell type-specific gene expression. In this manner, a cell's methylome can program the cell's terminal differentiation state to be, for instance, a neuron, a muscle cell, an immune cell, etc.

Bisulfite conversion or bisulfite sequencing may be used for DNA methylation analysis. Bisulfite sequencing is a convenient and effective method of mapping DNA methylation to individual bases. Unfortunately, bisulfite conversion is a harsh and destructive process for cfDNA that leads to degradation of >90% of the sample DNA.

Alternatively, enzymatic methylation (EM) conversion may be used for DNA methylation analysis and sequencing. In one embodiment, methylation conversion is mediated by non-destructive enzymatic reactions involving a ten-eleven translocation (TET) enzyme and a cytosine-deaminating enzyme (e.g., APOBEC) to convert unmethylated (but not methylated) cytosines to uracils. Other embodiments such as Tet-assisted pyridine borane sequencing (TAPS) combine enzymatic reactions such as TET together with chemical treatments (e.g., pyridine borane).

Examples of enzymatic methyl conversion workflows include enzymatic methyl-seq (EM-seq) and TET-assisted pyridine borane sequencing (TAPS).

EM-seq is a minimally destructive conversion methylation sequencing method for converting cytosines to uracils in nucleic acid. This bisulfite-free method preserves the length of nucleic acid molecules while achieving conversion rates similar to bisulfite sequencing. Further, EM-Seq can result in higher sequencing quality scores for cytosine and guanine base pairs, and can provide a more even coverage of various genomic features, such as CpG islands. EM-Seq comprises two sets of enzymatic reactions. In the initial reaction, a ten eleven translocation (TET) enzyme (e.g., TET1, TET2, TET3, Naegleria TET, and genetically engineered versions and/or variants thereof) and a β-glucosyltransferase (e.g., T4 BGT) convert 5mC and 5hmC into products that cannot be deaminated, or are resistant to deamination, by a cytosine-deaminating enzyme (e.g., APOBEC). In the second reaction, a cytosine-deaminating enzyme (e.g., APOBEC) deaminates unmodified (e.g., unmethylated) cytosines by converting them to uracils.

In another embodiment, TAPS can be used in enzymatic methylation sequencing workflows. TAPS is a minimally-destructive conversion methylation sequencing method for converting cytosines to uracil in nucleic acid. This bisulfite-free method allows minimal degradation of DNA, and thus preserves the length of nucleic acid molecules while achieving conversion rates similar to sodium bisulfite sequencing. TAPS can result in higher sequencing quality scores for cytosines and guanine base pairs, and can provide a more even coverage of various genomic features, such as CpG islands.

In TAPS, a ten eleven translocation enzyme (e.g., TET1) is used to oxidize both 5mC and 5hmC to 5caC. Pyridine borane is used to reduce 5caC to dihydrouracil, a uracil derivative that is then converted to thymine after PCR. TAPS can be performed in two other ways: TAPSß and chemical-assisted pyridine borane sequencing (CAPS). In TAPSB, β-glucosyltransferase is used to label 5hmC with glucose to protect 5hmC from the oxidation and reduction reactions and allows for specific detection of 5mC. In CAPS, potassium perruthenate acts as the chemical replacement for Tet1 and specifically oxidizes 5hmC, thus allowing for direct detection.

The advent of next generation DNA sequencing offers advances in clinical medicine and basic research. However, while this technology has the capacity to generate hundreds of billions of nucleotides of DNA sequence in a single experiment, the error rate of approximately 1% results in hundreds of millions of sequencing mistakes. Such errors can be tolerated in some applications but become extremely problematic for “deep sequencing” of genetically heterogeneous mixtures, such as tumors or mixed microbial populations. Thus, improved methods for analyzing methylation of cfDNA are needed to preserve the integrity of sample nucleic acid and enable improved accuracy of methylation state analysis at the whole genome or targeted level.

The method may include sequencing. The sequencing may be performed on a plurality of cfDNA fragments to generate a plurality of cfDNA sequencing fragments. In some cases, the cfDNA sequencing fragments may comprise more than or equal to 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 9,000, 10,00, 25,000, 50,000, or 100,000 cfDNA sequencing fragments. The cfDNA sequencing fragments may be various lengths (base pairs). In some cases, the cfDNA sequencing fragments have a length of more than or equal to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150,160, 170, 180, 190, 200, 210, 220, 230, 240, 250 260, 270, 280, 290, 300, 310, 320, 330, 340, or 350 base pairs. Each cfDNA sequencing fragments may comprise the same or different lengths in base pairs.

Non-limiting examples of sequencing include sequencing by synthesis (SBS), pyrosequencing, sequencing by ligation, sequencing by reversible terminator chemistry, phospholinked fluorescent nucleotide sequencing, and real-time sequencing. The method may include next generation sequencing (NGS). NGS utilizes the concept of massively parallel processing to obtain high-throughput, speed, and scalability. The methods may include RNA sequencing, such as mRNA sequencing, total RNA sequencing, low-input or ultra-low input RNA sequencing, small RNA sequencing, and single cell RNA sequencing. The methods may include DNA sequencing, such as sanger sequencing, capillary electrophoresis, sequencing by synthesis, shotgun sequencing, pyrosequencing, sequencing by ligation, nanopore sequencing, single molecular real time sequencing, and ion torrent sequencing, nanoball sequencing.

In various examples, enzymatic methylation sequencing results generates using the dsDNA library preparation methods described herein are used to analyze the methylation state of nucleic acids in a biological sample. In one example, whole genome enzymatic methyl sequencing (“WG EM-seq”) provides high resolution sequencing by characterizing DNA methylation of nearly every cytidine nucleotide in the genome. Other targeted methods, such as targeted enzymatic methyl sequencing (“TEM-seq”), may be useful for methylation analysis.

In other examples, assays that have conventionally been used for bisulfite conversion can be employed for minimally-destructive conversion methods, such as enzymatic conversion, TAPS, and CAPS. In various examples, assays used for methylation analysis may be mass spectrometry, methylation-specific PCR (MSP), reduced representation bisulfite sequencing (RRBS), HELP assay, GLAD-PCR assay, ChIP-on-chip assays, restriction landmark genomic scanning, methylated DNA immunoprecipitation (MeDIP), pyrosequencing of bisulfite treated DNA, molecular break light assay, methyl sensitive Southern Blotting, High Resolution Melt Analysis (HRM or HRMA), ancient DNA methylation reconstruction, or Methylation Sensitive Single Nucleotide Primer Extension Assay (msSNuPE).

The methylation profile of cfDNA can then be identified by applying sequence alignment methods to map methyl-seq reads from whole genome or targeted methyl sequencing of a human reference genome. Non-limiting examples of sequence alignment methods include bwa-meth, bismark, Last, GSNAP, BSMAP, NovoAlign, Bison, Metagenomic Phylogenetic Analysis (for example, MetaPhlAn2), BLAT, Burrows-Wheeler Aligner (BWA), Bowtie, Bowtie2, Bfast, BioScope, CLC bio, Cloudburst, Eland/Eland2, GenomeMapper, GnuMap, Karma, MAQ, MOM, Mosaik, MrFAST/MrsFAST, PASS, PerM, RazerS, RMAP, SSAHA2, Segemehl, SeqMap, SHRIMP, Slider/SliderII, Srprism, Stampy, vmatch, ZOOM, and the SOAP/SOAP2 alignment tool.

The method may include computer processing, and may include machine learning as disclosed in the machine learning section herein.

The method may include computer processing the plurality of cfDNA sequencing fragments.

In some embodiments, the computer processing comprises determining cfDNA fragmentation patterns in a plurality of cfDNA sequencing fragments.

In some embodiments, the cfDNA fragmentation patterns are determined based at least in part on transcription start-site gene activation probability.

In some embodiments, the method further comprises using the gene expression score to train a machine learning classifier capable of distinguishing between gene expression or gene non-expression. In some embodiments, targeted protein-coding genes covered in a DNA panel are divided into training and test (holdout) gene sets for the purpose of TSS-GAP featurization. The training set comprises a predefined list of “on” genes and “off” genes based upon known stably expressed genes with known expression states. In certain embodiments, the “off” genes are set forth in Table 1 and the “on” genes are set forth in Table 2 provided herein. In certain embodiments, “on” genes are housekeeping genes whereas “off” genes are set of genes that are known to be unexpressed based upon gene expression atlases (e.g., FANTOM5, ENCODE, EPD, VISTA or RefSeq databases). V-plots can be denoised using a Haar wavelet transform-based approach. The denoised V-plots corresponding to the training gene set can be used to train a linear or non-linear model to classify a V-plot (one per gene per sample) as “on” or “off”. The corresponding logistic regression probabilities can be used to generate gene activation (TSS-GAP) scores. In some embodiment, the TSS-GAP scores are defined as the probability of each holdout gene to be labeled as “on” (1) or off (0) by the trained classifier.

In some embodiments, the gene expression score comprises a value of between 0 and 1. In some examples, the gene expression score of 0 corresponds to non-expression of the gene. In some embodiments, the gene expression score of 1 corresponds to expression of the gene.

In some embodiments, the plurality of genes comprises epithelial cell-related genes.

In some embodiments, the plurality of genes comprises a member selected from the group consisting of: SOWAHB, TMEM63C, SOX2, TMEM184A, NBL1, B4GALNT2, TFAP2B, RND2, TP63, ATG9B, IGSF9, TMEM82, C10orf99, LOXL1, and GRB7. The gene may comprise SOWAHB. The gene may comprise TMEM63C. The gene may comprise SOX2. The gene may comprise TMEM184A. The gene may comprise NBL1. The gene may comprise B4GALNT2. The gene may comprise TFAP2B. The gene may comprise RND2. The gene may comprise TP63. The gene may comprise ATG9B. The gene may comprise IGSF9. The gene may comprise TMEM82. The gene may comprise C10orf99. The gene may comprise LOXL1. The gene may comprise GRB7.

In some embodiments, the method further comprises detecting the expression or non-expression of the gene with an accuracy. The accuracy may be expressed as a percentage. In some cases, the methods comprise detecting the expression or non-expression of a gene with an accuracy of more than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In another aspect, provided herein is a non-transitory computer-readable memory storing one or more instructions executable by one or more processors, that when executed by the one or more processors cause the one or more processors to perform processing comprising: obtaining a biological sample from a subject; extracting cell-free DNA (cfDNA) from the biological sample, wherein the cfDNA comprises a plurality of cfDNA fragments; performing a sequencing assay on the plurality of cfDNA fragments to generate a plurality of cfDNA sequencing fragments; computer processing the plurality of cfDNA sequencing fragments; and calculating, based at least in part on the computer processing, a gene expression score for a gene in a plurality of genes, wherein the gene expression score indicates a probability of expression or non-expression of the gene in the plurality of genes.

In another aspect, provided herein are computer systems for inferring gene expression, the system comprising: a non-transitory memory; and a processor in communication with the non-transitory memory, the processor configured to execute the following operations in order to effectuate a method comprising the operations of: obtaining a biological sample from a subject; extracting cell-free DNA (cfDNA) from the biological sample, wherein the cfDNA comprises a plurality of cfDNA fragments; performing a sequencing assay on the plurality of cfDNA fragments to generate a plurality of cfDNA sequencing fragments; computer processing the plurality of cfDNA sequencing fragments; and calculating, based at least in part on the computer processing, a gene expression score for a gene in a plurality of genes, wherein the gene expression score indicates a probability of expression or non-expression of the gene in the plurality of genes.

In an aspect, the present disclosure provides methods for inferring gene expression. The methods may comprise preparing a methylation sequencing library. The methods may comprise obtaining a biological sample from a subject. The methods may comprise extracting cell-free deoxyribonucleic acid (cfDNA) from a biological sample. The cfDNA may comprises a plurality of cfDNA fragments, The methods may comprise providing conditions capable of converting unmethylated cytosines to uracils in cfDNA fragments to produce a plurality of converted cfDNA fragments. The methods may comprise enriching the converted cfDNA fragments to produce enriched converted cfDNA fragment molecules. The enriching may comprise contacting the converted cfDNA fragments with a probe set comprising hybridization probes having sequence complementarity to transcription start site (TSS) sequences of a plurality of TSS. The methods may comprise amplifying the enriched converted cfDNA fragment molecules to produce amplified enriched converted cfDNA fragments. The methods may comprise determining a nucleic acid sequence of a plurality of cfDNA sequencing fragments of the amplified enriched converted cfDNA fragments. The methods may comprise processing the plurality of cfDNA sequencing fragments. The processing may comprise calculating a gene expression score for one or more genes of a plurality of genes. The genes expression score may indicate a probability of expression or non-expression of the one or more genes of the plurality of genes.

In an aspect, the present disclosure provides methods for preparing a methylation sequencing library for inferring gene expression. The methods may comprise obtaining a biological sample from a subject. The methods may comprise extracting cell-free deoxyribonucleic acid (cfDNA) from the biological sample. The cfDNA may comprise a plurality of cfDNA fragments. The methods may comprise providing conditions capable of converting unmethylated cytosines to uracils in the cfDNA fragments to produce a plurality of converted cfDNA fragment molecules. In an alternative embodiment, the methods may comprise providing conditions capable of converting methylated cytosines to thymine in the cfDNA fragments to produce a plurality of converted cfDNA fragment molecules. The methods may comprise amplifying the plurality of converted cfDNA fragment molecules to produce amplified converted cfDNA fragments. The methods may comprise determining a nucleic acid sequence of a plurality of cfDNA sequencing fragments of the amplified converted cfDNA fragments. The methods may comprise processing the plurality of cfDNA sequencing fragments. The processing may comprise calculating a gene expression score for one or more genes in a plurality of genes. The gene expression score may indicate a probability of expression or non-expression of the one or more genes of the plurality of genes.

In an aspect, the present disclosure provides methods for preparing a sequencing library for inferring gene expression. The methods may comprise obtaining a biological sample from a subject. The methods may comprise extracting cell-free deoxyribonucleic acid (cfDNA) from the biological sample. The cfDNA may comprise a plurality of cfDNA fragments. The methods may comprise enriching the plurality of cfDNA fragments to produce enriched cfDNA fragment molecules. The enriching may comprise contacting the plurality of cfDNA fragments with a probe set comprising hybridization probes having sequence complementarity to at least two transcription start site (TSS) sequences of a plurality of TSS selected from the genes listed in Tables 1-5. The methods may comprise amplifying the enriched cfDNA fragment molecules to produce amplified enriched cfDNA fragments. The methods may comprise determining a nucleic acid sequence of a plurality of cfDNA sequencing fragments of the amplified enriched cfDNA fragments. The methods may comprise processing the plurality of sequenced enriched cfDNA fragments. The processing may comprise calculating a gene expression score for one or more genes in a plurality of genes. The gene expression score may indicate a probability of expression or non-expression of the one or more genes of the plurality of genes.

In an aspect, the present disclosure provides methods for preparing a sequencing library for inferring gene expression. The methods may comprise obtaining a biological sample from a subject. The methods may comprise extracting cell-free deoxyribonucleic acid (cfDNA) from the biological sample, wherein the cfDNA comprises a plurality of cfDNA fragments. The methods may comprise amplifying the plurality of cfDNA fragments to produce amplified cfDNA fragments. The methods may comprise determining a nucleic acid sequence of a plurality of cfDNA sequencing fragments of the amplified cfDNA fragments. The methods may comprise processing the plurality of sequenced cfDNA fragments. The processing may comprise calculating a gene expression score for one or more genes in a plurality of genes. The gene expression score may indicate a probability of expression or non-expression of the one or more genes of the plurality of genes.

The methods may comprise obtaining a biological sample. The biological sample may be obtained from a subject. The biological sample may be derived from a subject. The biological sample may comprise a blood sample. The biological sample may comprise a cellular sample. The biological sample may comprise a blood sample and a cellular sample. The blood sample may comprise a plasma sample, a serum sample, or a buffy coat sample. The blood sample may comprise a plasma sample. The blood sample may comprise a serum sample. The blood sample may comprise a buffy coat sample. The cellular sample may comprise a tissue sample, isolated cells, a biopsy sample, or a plurality of cells from a cell line. The cellular sample may comprise a tissue sample. The cellular sample may comprise a biopsy sample. The cellular sample may comprise a plurality of cells from a cell line. The cellular sample may comprise a plurality of cells from one or more cell lines. The cellular sample may comprise cells isolated from the blood. The biological sample may comprise nucleic acids, for example, deoxyribonucleic acid (DNA), ribonucleic acid (RNA), or a combination thereof. The biological sample may comprise a tissue or a cell.

The methods may comprise obtaining a biological sample from the subject. The biological sample may be obtained or derived from the subject. The subject may be a mammal. For example, the subject may be a human or a non-human mammal. The subject may be a dog, a pig, a sheep, a cow, a goat, or a feline. The subject may be an adult (e.g., a human 18 years of age or older). The subject may be a child (e.g., a human less than 18 years of age). The subject may be a fish. The subject may be a reptile. The subject may be a rodent, for example, a rat, a mouse, a guinea pig, or a hamster. The subject may be a mammal. In some embodiments, the subject is human.

The methods may comprise extracting cell-free deoxyribonucleic acid (cfDNA) from a biological sample. The cfDNA may comprise a plurality of cfDNA fragment. The plurality of cfDNA fragments may comprise about 3, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 550, about 600, about 650, about 700, about 750, about 800, about 850, about 900, about 950, about 1,000, about 2,000, about 3,000, about 4,000, about 5,000, about 6,000, about 7,000, about 8,000, about 9,000, or about 10,000 cfDNA fragments.

The methods may comprise providing conditions capable of converting unmethylated cytosines to uracils in cfDNA fragments to produce a plurality of converted cfDNA fragments. The conditions may comprise a temperature or a change in temperature. The conditions may comprise providing one or more agents to the biological sample in various environmental conditions.

The methods may comprise enriching. In some embodiments, the enriching may comprise enriching cfDNA fragments to produce enriched cfDNA fragment molecules. The cfDNA may comprise converted cfDNA fragments. The cfDNA may not comprise converted cfDNA fragments. The cfDNA may have undergone a conversion operation. The cfDNA may not have undergone a conversion operation. In some embodiments, the enriching may comprise enriching converted or non-converted cfDNA fragments to produce enriched converted or enriched non-converted cfDNA fragment molecules. The enriching may comprise use of hybridization probes. The enriching may comprise use of centrifugation. The enriching may comprise use of an immunoassay. The enrichment may comprise use of beads, for example, magnetic beads. The enriching may comprise use of antibodies.

The methods may comprise providing one or more nucleases. In some embodiments, the methods may comprise treating a biological sample with one or more nucleases. The methods may comprise treating deoxyribonucleic acid (DNA) from a biological sample with one or more nucleases. The treatment with nucleases may be performed prior to an enriching of cfDNA fragments. The treatment with nucleases may be performed simultaneously with an enriching of cfDNA fragments. The treatment with nucleases may be performed substantially simultaneously with an enriching of cfDNA fragments. The treatment with nucleases may be performed after an enriching of cfDNA fragments. In some embodiments, the one or more nucleases may comprise micrococcal nuclease (MNase) or deoxyribonuclease (DNase). The DNase may comprise DNase I or DNase II. In some embodiments, the one or more nucleases may comprise exonucleases, nuclease S1, endonucleases, lambda exonucleases, ribonucleases, micrococcal nucleases, mung bean nucleases, or a combination thereof. In some embodiments, the methods may comprise providing one or more, two or more, three or more, four or more, five or more, six or more, seven or more, eight or more, nine or more, or ten or more nucleases.

In some embodiments, the methods may comprise obtaining a biological sample from a subject; extracting cell-free deoxyribonucleic acid (cfDNA) and/or nuclease-treated DNA from the biological sample, wherein the cfDNA comprises a plurality of cfDNA fragments; providing conditions capable of converting unmethylated cytosines to uracils in the cfDNA fragments to produce a plurality of converted cfDNA fragments; enriching the converted cfDNA fragments to produce enriched converted cfDNA fragment molecules, wherein the enriching comprises contacting the converted cfDNA fragments with a probe set comprising hybridization probes having sequence complementarity to at least two transcription start site (TSS) sequences of a plurality of TSS sequences; amplifying the enriched converted cfDNA fragment molecules to produce amplified enriched converted cfDNA fragments; and determining a nucleic acid sequence of a plurality of cfDNA sequencing fragments of the amplified enriched converted cfDNA fragments.

In some embodiments, the methods may comprise obtaining a biological sample from a subject; extracting cell-free deoxyribonucleic acid (cfDNA) and/or nuclease-treated DNA from the biological sample, wherein the cfDNA comprises a plurality of cfDNA fragments; enriching the cfDNA fragments to produce enriched cfDNA fragment molecules, wherein the enriching comprises contacting the cfDNA fragments with a probe set comprising hybridization probes having sequence complementarity to at least two transcription start site (TSS) sequences of a plurality of TSS sequences; amplifying the enriched cfDNA fragment molecules to produce amplified enriched cfDNA fragments; and determining a nucleic acid sequence of a plurality of cfDNA sequencing fragments of the amplified enriched cfDNA fragments.

The methods may make use of a probe set comprising hybridization probes. The hybridization probes may have complementarity to transcription start site (TSS) sequences of a plurality of TSS. The hybridization probes may be complementary to TSS sequences of a set of genes. The hybridization probes may be substantially complementary to TSS sequences of a set of genes. The hybridization probes may be partially complementary to TSS sequences of a set of genes.

The methods may comprise contacting cfDNA fragments with a probe set comprising hybridization probes. The hybridization probes may comprise sequence complementarity to TSS sequences of a plurality of TSS sequences. The TSS sequences may be from a set of genes. The plurality of TSS sequences may be selected from the group consisting of the TSS sequences listed in the genes of Tables 1-5.

In some embodiments, the plurality of TSS sequences may comprise TSS sequences of genes listed in Table 1. The plurality of TSS sequence may comprise more than or equal to about 3, about 4, about 5, about 6, about 7, about 8, about 9, about 10, about 25, about 50, about 75, about 100, about 150, about 200, about 250, about 300, about 350, about 400, about 450, about 500, about 550, or about 595 TSS sequences of genes listed in Table 1. The plurality of TSS sequence may comprise less than or equal to about 595, about 550, about 500, about 450, about 400, about 350, about 300, about 250, about 200, about 150, about 100, about 75, about 50, about 25, about 10, about 9, about 8, about 7, about 6, about 5, about 4, or about 3 TSS sequences of genes listed in Table 1.

In some embodiments, the plurality of TSS sequences may comprise TSS sequences of genes listed in Table 2. The plurality of TSS sequence may comprise more than or equal to about 3, about 4, about 5, about 6, about 7, about 8, about 9, about 10, about 25, about 50, about 75, about 100, about 150, about 200, about 250, about 300, about 350, about 400, about 450, about 500, about 550, or about 595 TSS sequences of genes listed in Table 2. The plurality of TSS sequence may comprise less than or equal to about 595, about 550, about 500, about 450, about 400, about 350, about 300, about 250, about 200, about 150, about 100, about 75, about 50, about 25, about 10, about 9, about 8, about 7, about 6, about 5, about 4, or about 3 TSS sequences of genes listed in Table 2.

In some embodiments, the plurality of TSS sequences may comprise TSS sequences of genes listed in Table 3. The plurality of TSS sequence may comprise more than or equal to about 3, about 4, about 5, about 6, about 7, about 8, about 9, about 10, about 25, about 50, about 75, about 100, about 150, about 200, about 250, about 300, about 350, about 400, about 450, about 500, about 550, about 600, about 650, about 700, about 750, about 800, about 850, or about 890 TSS sequences of genes listed in Table 3. The plurality of TSS sequence may comprise less than or equal to about 890, about 850, about 800, about 750, about 700, about 650, about 600, about 550, about 500, about 450, about 400, about 350, about 300, about 250, about 200, about 150, about 100, about 75, about 50, about 25, about 10, about 9, about 8, about 7, about 6, about 5, about 4, or about 3 TSS sequences of genes listed in Table 3.

In some embodiments, the plurality of TSS sequences may comprise TSS sequences of genes listed in Table 4. The plurality of TSS sequence may comprise more than or equal to about 3, about 4, about 5, about 6, about 7, about 8, about 9, about 10, about 25, about 50, about 75, about 100, about 150, about 200, about 250, about 300, about 350, about 400, about 450, about 500, about 550, about 600, about 650, about 700, about 750, about 800, about 850, about 900, about 1,000, about 1,050, about 1,100, about 1,150, about 1,200, about 1,250, or about 1,296 TSS sequences of genes listed in Table 4. The plurality of TSS sequence may comprise less than or equal to about 1,250, about 1,200, about 1,150, about 1,100, about 1,050, about 1,000, about 950, about 900, about 850, about 800, about 750, about 700, about 650, about 600, about 550, about 500, about 450, about 400, about 350, about 300, about 250, about 200, about 150, about 100, about 75, about 50, about 25, about 10, about 9, about 8, about 7, about 6, about 5, about 4, or about 3 TSS sequences of genes listed in Table 4.

In some embodiments, the plurality of TSS sequences may comprise TSS sequences of genes listed in Table 5. The plurality of TSS sequence may comprise more than or equal to about 3, about 4, about 5, about 6, about 7, about 8, about 9, about 10, about 25, about 50, about 75, about 100, about 150, about 200, about 250, about 300, about 350, about 400, about 450, about 500, about 550, about 600, about 650, about 700, about 750, about 800, about 850, about 900, about 1,000, about 1,050, about 1,100, about 1,150, about 1,200, about 1,250, about 1,300, about 1,350, about 1,400, about 1,450, about 1,500, about 1,600, about 1,700, about 1,800, about 1,900, about 2,000, about 2,100, about 2,200, about 2,300, about 2,400, about 2,500, about 2,600, about 2,700, about 2,800, about 2,900, about 3,000 about 3,100, about 3,200, about 3,300, about 3,400, about 3,500, about 3,600, about 3,700, about 3,800, about 3,900, about 4,000, about 4,100, about 4,200, about 4,300, about 4,400, about 4,500, about 4,600, about 4,700, about 4,800, about 4,900, about 5,000, about 5,100, about 5,200, about 5,300, about 5,400, about 5,500, about 5,600, about 5,700, about 5,800, about 5,900, about 6,000, about 6,100, about 6,200, about 6,300, about 6,400, about 6,500, about 6,600, about 6,700, about 6,800, about 6,900, about 7,000, about 7,100, about 7,200, about 7,300, about 7,400, about 7,500, about 7,600, about 7,700, about 7,800, about 7,900, about 8,000, about 8,100, about 8,200, about 8,300, about 8,400, about 8,500, about 8,600, about 8,700, about 8,800, about 8,900, about 9,000, about 9,100, about 9,200, about 9,300, about 9,400, about 9,500, about 9,600, about 9,700, about 9,800, about 9,900, about 10,000, about 10,250 about 10,500, about 10,750 about 11,000 about 11,250, about 11,500, about 11,750 about 12,000, about 12,250 about 12,500, about 12,750 about 13,000 about 13,250 about 13,500, about 13,750 about 14,000, about 14,250 about 14,500, about 14,750 about 15,000, about 15,250 about 15,500, about 15,750 about 16,000, about 16,250, about 16,500, about 16,750 about 17,000, about 17,250, about 17,500, about 17,750 about 18,000, about 18,250, about 18,500, or about 18,721 TSS sequences of genes listed in Table 5. The plurality of TSS sequence may comprise less than or equal to about 18,721, about 18,500, about 18,250, about 18,000, about 17,750, about 17,500, about 17,250, about 17,000, about 16,750, about 16,500, about 16,250, about 16,000, about 15,750, about 15,500, about 15,250, about 15,000, about 14,750, about 14,500, about 14,250, about 14,000, about 13,750, about 13,500, about 13,250, about 13,000, about 12,750, about 12,500, about 12,250, about 12,000, about 11,750, about 11,500, about 11,250, about 11,000, about 10,750, about 10,500, about 10,250, about 10,000, about 9,900, about 9,800, about 9,700, about 9,600, about 9,500, about 9,400, about 9,300, about 9,200, about 9,100, about 9,000, about 8,900, about 8,800, about 8,700, about 8,600, about 8,500, about 8,400, about 8,300, about 8,200, about 8,100, about 8,000, about 7,900, about 7,800, about 7,700, about 7,600, about 7,500, about 7,400, about 7,300, about 7,200, about 7,100, about 7,000, about 6,900, about 6,800, about 6,700, about 6,600, about 6,500, about 6,400, about 6,300, about 6,200, about 6,100, about 6,000, about 5,900, about 5,800, about 5,700, about 5,600, about 5,500, about 5,400, about 5,300, about 5,200, about 5,100, about 5,000, about 4,900, about 4,800, about 4,700, about 4,600, about 4,500, about 4,400, about 4,300, about 4,200, about 4,100, about 4,000, about 3,900, about 3,800, about 3,700, about 3,600, about 3,500, about 3,400, about 3,300, about 3,200, about 3,100, about 3,000, about 2,900, about 2,800, about 2,700, about 2,600, about 2,500, about 2,400, about 2,300, about 2,200, about 2,100, about 2,000, about 1,900, about 1,800, about 1,700, about 1,600, about 1,500, about 1,400, about 1,300, about 1,200, about 1,100, about 1,000, about 950, about 900, about 850, about 800, about 750, about 700, about 650, about 600, about 550, about 500, about 450, about 400, about 350, about 300, about 250, about 200, about 150, about 100, about 75, about 50, about 25, about 10, about 9, about 8, about 7, about 6, about 5, about 4, or about 3 TSS sequences of genes listed in Table 5.

In some embodiments, the plurality of TSS sequences may comprise TSS sequences of genes listed in Table 1, Table 2, Table 3, Table 4, Table 5, or any combination thereof. The plurality of TSS sequences may comprise TSS sequences of genes listed in Table 1 and Table 2. The plurality of TSS sequences may comprise TSS sequences of genes listed in Table 1 and Table 3. The plurality of TSS sequences may comprise TSS sequences of genes listed in Table 1 and Table 4. The plurality of TSS sequences may comprise TSS sequences of genes listed in Table 1 and Table 5. The plurality of TSS sequences may comprise TSS sequences of genes listed in Table 2 and Table 3. The plurality of TSS sequences may comprise TSS sequences of genes listed in Table 2 and Table 4. The plurality of TSS sequences may comprise TSS sequences of genes listed in Table 2 and Table 5. The plurality of TSS sequences may comprise TSS sequences of genes listed in Table 3 and Table 4. The plurality of TSS sequences may comprise TSS sequences of genes listed in Table 3 and Table 5. The plurality of TSS sequences may comprise TSS sequences of genes listed in Table 4 and Table 5.

The probe set comprising hybridization probes may comprise one or more hybridization probes. In some embodiments, the probe set comprising hybridization probes may comprise more than or equal to about 1 hybridization probe, about 2 hybridization probes, about 3 hybridization probes, about 4 hybridization probes, about 5 hybridization probes, about 10 hybridization probes, about 25 hybridization probes, about 50 hybridization probes, about 100 hybridization probes, about 150 hybridization probes, about 200 hybridization probes, about 250 hybridization probes, about 300 hybridization probes, about 350 hybridization probes, about 400 hybridization probes, about 450 hybridization probes, about 500 hybridization probes, about 550 hybridization probes, about 600 hybridization probes, about 650 hybridization probes, about 700 hybridization probes, about 750 hybridization probes, about 800 hybridization probes, about 850 hybridization probes, about 900 hybridization probes, about 950 hybridization probes, about 1,000 hybridization probes, about 1,500 hybridization probes, about 2,000 hybridization probes, about 3,000 hybridization probes, about 4,000 hybridization probes, about 5,000 hybridization probes, about 6,000 hybridization probes, about 7,000 hybridization probes, about 8,000 hybridization probes, about 9,000 hybridization probes, or about 10,000 hybridization probes. In some embodiments, the probe set comprising hybridization probes may comprise less than or equal to about 10,000 hybridization probes, about 9,000 hybridization probes, about 8,000 hybridization probes, about 7,000 hybridization probes, about 6,000 hybridization probes, about 5,000 hybridization probes, about 4,000 hybridization probes, about 3,000 hybridization probes, about 2,000 hybridization probes, about 1,500 hybridization probes, about 1,000 hybridization probes, about 950 hybridization probes, about 900 hybridization probes, about 850 hybridization probes, about 800 hybridization probes, about 750 hybridization probes, about 700 hybridization probes, about 650 hybridization probes, about 600 hybridization probes, about 550 hybridization probes, about 500 hybridization probes, about 450 hybridization probes, about 400 hybridization probes, about 350 hybridization probes, about 300 hybridization probes, about 250 hybridization probes, about 200 hybridization probes, about 150 hybridization probes, about 100 hybridization probes, about 90 hybridization probes, about 80 hybridization probes, about 70 hybridization probes, about 60 hybridization probes, about 50 hybridization probes, about 40 hybridization probes, about 30 hybridization probes, about 20 hybridization probes, about 10 hybridization probes, about 8 hybridization probes, about 6 hybridization probes, about 4 hybridization probes, about 3 hybridization probes, or about 2 hybridization probes.

The hybridization probes may be designed to cover regions flanking TSS sequences. In some embodiments, the hybridization probes may be designed to cover a 1,500-base pair (bp) window (e.g., 750 bp downstream a TSS sequence and 750 bp upstream a TSS sequence). In some embodiments, the bp window is about 500 bp, about 600 bp, about 700 bp, about 800 bp, about 900 bp, about 1,000 bp, about 1,100 bp, about 1,150 bp, about 1,200 bp, about 1,250 bp, about 1,300 bp, about 1,400 bp, about 1,500 bp, about 1,600 bp, about 1,700 bp, about 1,800 bp, about 1,900 bp, about 2,000 bp, about 2,100 bp, about 2,200 bp, about 2,300 bp, about 2,400 bp, about 2,500 bp, about 2,600 bp, about 2,700 bp, about 2,800 bp, about 2,900 bp, about 3,000 bp, about 3,100 bp, about 3,200 bp, about 3,300 bp, about 3,400 bp, about 3,500 bp, or about 3,600 bp.

The methods may comprise performing a sequencing assay. The sequencing assay may be performed on a plurality of cfDNA fragments. In some embodiments, the sequencing assay may comprise next generation sequencing (NGS). The NGS may comprise whole genome sequencing (WGS). The NGS may comprise targeted sequencing. The NGS may comprise WGS and/or targeted sequencing. The sequencing assay may comprise bisulfite conversion, enzymatic conversion, or TET-assisted pyridine borane sequencing (TAPS) conversion. The sequencing assay may comprise bisulfite conversion. The sequencing assay may comprise enzymatic conversion. The sequencing assay may comprise TET-assisted pyridine borane sequencing (TAPS) conversion. The sequencing assay may comprise ribonucleic acid (RNA) sequencing, polony sequencing, SOLiD sequencing, Sanger sequencing, deoxyribonucleic acid (DNA) sequencing, cycle sequencing, pyrosequencing, amplicon sequencing, exome sequencing nanopore sequencing, Shotgun sequencing, ChiP-seq, or a combination thereof.

The methods may comprise determining fragmentation patterns. The fragmentation patterns may be determined in a plurality of cfDNA sequencing fragments. The methods may comprise using the fragmentation patterns to train a machine learning classifier. The machine learning classifier may be capable of distinguishing between gene expression and gene non-expression of one or more genes. The machine learning classifier may comprise any machine learning classifier disclosed herein.

The methods may comprise calculating a gene expression score. The gene expression score may be for one or more genes of a plurality of genes. The genes expression score may indicate a probability of expression or non-expression of the one or more genes of the plurality of genes. The gene expression score may comprise a numerical value. The gene expression score may comprise a numerical value of about from 0 to about 1. In some embodiments, the gene expression score may be about 0.0, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, about 0.99, or about 1.0. In some embodiments, a gene expression score of 0 corresponds to non-expression of a gene. In some embodiments, a gene expression score of about 0 to about 0.49 corresponds to a likelihood of non-expression of a gene. In some embodiments, a gene expression score of about 0.0, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, or about 0.49 corresponds to a likelihood of non-expression of a gene. In some embodiments, a gene expression score of 1 corresponds to expression of a gene. In some embodiments, a gene expression score of about 0.51 to about 1.0 corresponds to a likelihood of expression of a gene. In some embodiments, a gene expression score of about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, about 0.99, or about 1.0 corresponds to a likelihood of expression of a gene.

In some embodiments, the gene expression score may be used to distinguish between two samples. In some embodiments, the gene expression score may be used to distinguish between a diseased sample and healthy sample. The diseased sample may be obtained or derived from a subject having a disease. The disease may be cancer. The healthy sample may be obtained or derived from a subject not having cancer.

In some embodiments, the cancer may be selected from the group consisting of breast cancer, diffuse large B cell lymphoma, esophageal cancer, lung cancer, pancreatic cancer, liver cancer, colorectal cancer, prostate cancer, gastric cancer, ovarian cancer, and bile duct cancer. The cancer may be pre-cancer lesions, stage 0, stage I cancer, stage II cancer, stage III cancer, or stage IV cancer.

The methods may comprise detecting the expression or non-expression of one or more genes. In some embodiments, the methods comprise detecting the expression or non-expression or more than or equal to about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 225, about 250, about 275, about 300, about 325, about 350, about 375, about 400, about 425, about 450, about 475, about or about 500 genes. In some embodiments, the methods comprise detecting the expression or non-expression or less than or equal to about 500, about 475, about 450, about 425, about 400, about 375, about 350, about 325, about 300, about 275, about 250, about 225, about 200, about 175, about 150, about 125, about 100, about 95, about 90, about 85, about 80, about 75, about 70, about 65, about 60, about 55, about 50, about 45, about 40, about 35, about 30, about 25, about 20, about 15, about 10, about 9, about 8, about 7, about 6, about 5, about 4, about 3, about 2, or about 1 genes.

In some embodiments, the expression or non-expression of one or more genes may be determined with an accuracy of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

In some embodiments, the expression or non-expression of one or more genes may be determined with a sensitivity of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

In some embodiments, the expression or non-expression of one or more genes may be determined with a specificity of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

In some embodiments, the expression or non-expression of one or more genes may be determined with a positive predictive value (PPV) of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

In some embodiments, the expression or non-expression of one or more genes may be determined with a negative predictive value (NPV) of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

In some embodiments, the expression or non-expression of one or more genes may be determined with an Area Under the Curve (AUC) value of a Receiver Operating Characteristic (ROC) of at least about 0.50, about 0.55, about 0.60, about 0.65, about 0.70, about 0.75, about 0.80, about 0.85, about 0.90, about 0.95, about 0.96, about 0.97, about 0.98, or about 0.99.

The methods may comprise using one or more gene panels. In some embodiments, the methods may comprise using the gene panel of Table 1 (“training genes (off”). In some embodiments, the methods may comprise using the gene panel of Table 2 (“training genes (on)”). In some embodiments, the methods may comprise using the gene panel of Table 3 (Cancer Panel 1). In some embodiments, the methods may comprise using the gene panel of Table 4 (Cancer Panel 2). In some embodiments, the methods may comprise using the gene panel of Table 5 (“target genes”).

The methods may comprise training a machine leaning model with gene panels. In some embodiments, the methods may comprise training a machine learning model with the 595 genes in Table 1. Table 1 provides a list of genes, including the Ensembl ID and the gene symbol. The genes in Table 1 may be used as “off”′ training genes with a gene expression score of 0.

In some embodiments, the methods may comprise training a machine learning model with the 595 genes in Table 2. Table 2 provide a list of genes, including the Ensembl ID and the gene symbol. The genes in Table 2 may be used as “on” training genes with a gene expression score of 1.

Table 3 provides a first list of genes relating to cancer, including the gene name and the hedge's g value. Table 4 provides a second list of genes related to cancer, including the gene name and the hedge's g value. The hedge's g corresponds to the effect size value of each gene.

TABLE 1
Gene Panel: Training Genes (off)

| OFF Training Gene Ensembl ID | Gene Symbol |
|---|---|
| ENSG00000114771 | AADAC |
| ENSG00000188984 | AADACL3 |
| ENSG00000205002 | AARD |
| ENSG00000138075 | ABCG5 |
| ENSG00000115361 | ACADL |
| ENSG00000153093 | ACOXL |
| ENSG00000183760 | ACP7 |
| ENSG00000159251 | ACTC1 |
| ENSG00000164113 | ADAD1 |
| ENSG00000168619 | ADAM18 |
| ENSG00000168594 | ADAM29 |
| ENSG00000151388 | ADAMTS12 |
| ENSG00000140873 | ADAMTS18 |
| ENSG00000145808 | ADAMTS19 |
| ENSG00000118492 | ADGB |
| ENSG00000164393 | ADGRF2 |
| ENSG00000164199 | ADGRV1 |
| ENSG00000182035 | ADIG |
| ENSG00000120907 | ADRA1A |
| ENSG00000187546 | AGMO |
| ENSG00000113492 | AGXT2 |
| ENSG00000145192 | AHSG |
| ENSG00000129221 | AIPL1 |
| ENSG00000122787 | AKR1D1 |
| ENSG00000108602 | ALDH3A1 |
| ENSG00000132746 | ALDH3B2 |
| ENSG00000196711 | ALKAL1 |
| ENSG00000151360 | ALLC |
| ENSG00000106927 | AMBP |
| ENSG00000178171 | AMER3 |
| ENSG00000116748 | AMPD1 |
| ENSG00000187689 | AMTN |
| ENSG00000153930 | ANKFN1 |
| ENSG00000165887 | ANKRD2 |
| ENSG00000180777 | ANKRD30B |
| ENSG00000163046 | ANKRD30BL |
| ENSG00000167612 | ANKRD33 |
| ENSG00000181626 | ANKRD62 |
| ENSG00000110243 | APOA5 |
| ENSG00000110245 | APOC3 |
| ENSG00000088756 | ARHGAP28 |
| ENSG00000124143 | ARHGAP40 |
| ENSG00000164122 | ASB5 |
| ENSG00000154438 | ASZ1 |
| ENSG00000107518 | ATRNL1 |
| ENSG00000135454 | B4GALNT1 |
| ENSG00000136881 | BAAT |
| ENSG00000132692 | BCAN |
| ENSG00000127325 | BEST3 |
| ENSG00000198183 | BPIFA1 |
| ENSG00000131059 | BPIFA3 |
| ENSG00000125999 | BPIFB1 |
| ENSG00000184459 | BPIFC |
| ENSG00000137948 | BRDT |
| ENSG00000078725 | BRINP1 |
| ENSG00000138152 | BTBD16 |
| ENSG00000178645 | C10orf53 |
| ENSG00000177354 | C10orf71 |
| ENSG00000154493 | C10orf90 |
| ENSG00000176029 | C11orf16 |
| ENSG00000180999 | C1orf105 |
| ENSG00000162598 | C1orf87 |
| ENSG00000240654 | C1QTNF9 |
| ENSG00000258713 | C20orf141 |
| ENSG00000205856 | C22orf42 |
| ENSG00000138813 | C4orf17 |
| ENSG00000204661 | C5orf60 |
| ENSG00000039537 | C6 |
| ENSG00000021852 | C8B |
| ENSG00000154975 | CA10 |
| ENSG00000174990 | CA5A |
| ENSG00000157782 | CABP1 |
| ENSG00000006283 | CACNA1G |
| ENSG00000175161 | CADM2 |
| ENSG00000110680 | CALCA |
| ENSG00000004948 | CALCR |
| ENSG00000070808 | CAMK2A |
| ENSG00000182533 | CAV3 |
| ENSG00000174015 | CBY2 |
| ENSG00000185860 | CCDC190 |
| ENSG00000162592 | CCDC27 |
| ENSG00000183273 | CCDC60 |
| ENSG00000173093 | CCDC63 |
| ENSG00000152076 | CCDC74B |
| ENSG00000150676 | CCDC83 |
| ENSG00000187094 | CCK |
| ENSG00000110148 | CCKBR |
| ENSG00000064205 | CCN5 |
| ENSG00000161649 | CD300LG |
| ENSG00000040731 | CDH10 |
| ENSG00000145526 | CDH18 |
| ENSG00000150394 | CDH8 |
| ENSG00000099954 | CECR2 |
| ENSG00000142789 | CELA3A |
| ENSG00000219073 | CELA3B |
| ENSG00000137691 | CFAP300 |
| ENSG00000181378 | CFAP65 |
| ENSG00000134365 | CFHR4 |
| ENSG00000135346 | CGA |
| ENSG00000141371 | CHCT1 |
| ENSG00000134216 | CHIA |
| ENSG00000120903 | CHRNA2 |
| ENSG00000147432 | CHRNB3 |
| ENSG00000117971 | CHRNB4 |
| ENSG00000157884 | CIB4 |
| ENSG00000153789 | CIBAR2 |
| ENSG00000137975 | CLCA2 |
| ENSG00000013297 | CLDN11 |
| ENSG00000066405 | CLDN18 |
| ENSG00000184697 | CLDN6 |
| ENSG00000188393 | CLEC2A |
| ENSG00000104938 | CLEC4M |
| ENSG00000137392 | CLPS |
| ENSG00000146352 | CLVS2 |
| ENSG00000183671 | CMKLR2 |
| ENSG00000144191 | CNGA3 |
| ENSG00000146910 | CNPY1 |
| ENSG00000149972 | CNTN5 |
| ENSG00000134115 | CNTN6 |
| ENSG00000152910 | CNTNAP4 |
| ENSG00000124749 | COL21A1 |
| ENSG00000144810 | COL8A1 |
| ENSG00000112280 | COL9A1 |
| ENSG00000184374 | COLEC10 |
| ENSG00000156885 | COX6A2 |
| ENSG00000170516 | COX7B2 |
| ENSG00000091704 | CPA1 |
| ENSG00000128510 | CPA4 |
| ENSG00000120054 | CPN1 |
| ENSG00000178772 | CPN2 |
| ENSG00000196353 | CPNE4 |
| ENSG00000132693 | CRP |
| ENSG00000105392 | CRX |
| ENSG00000163499 | CRYBA2 |
| ENSG00000136488 | CSH1 |
| ENSG00000204414 | CSHL1 |
| ENSG00000121904 | CSMD2 |
| ENSG00000164796 | CSMD3 |
| ENSG00000225362 | CT62 |
| ENSG00000066032 | CTNNA2 |
| ENSG00000168925 | CTRB1 |
| ENSG00000160882 | CYP11B1 |
| ENSG00000095596 | CYP26A1 |
| ENSG00000138109 | CYP2C9 |
| ENSG00000187048 | CYP4A11 |
| ENSG00000162365 | CYP4A22 |
| ENSG00000110887 | DAO |
| ENSG00000187323 | DCC |
| ENSG00000180483 | DEFB119 |
| ENSG00000157680 | DGKI |
| ENSG00000100867 | DHRS2 |
| ENSG00000104901 | DKKL1 |
| ENSG00000147570 | DNAJC5B |
| ENSG00000015413 | DPEP1 |
| ENSG00000130226 | DPP6 |
| ENSG00000134762 | DSC3 |
| ENSG00000134760 | DSG1 |
| ENSG00000221818 | EBF2 |
| ENSG00000164318 | EGFLAM |
| ENSG00000196361 | ELAVL3 |
| ENSG00000110675 | ELMOD1 |
| ENSG00000169393 | ELSPBP1 |
| ENSG00000135063 | ENTREP1 |
| ENSG00000101448 | EPPIN |
| ENSG00000164283 | ESM1 |
| ENSG00000196482 | ESRRG |
| ENSG00000164089 | ETNPPL |
| ENSG00000006468 | ETV1 |
| ENSG00000178997 | EXD1 |
| ENSG00000180210 | F2 |
| ENSG00000164434 | FABP7 |
| ENSG00000148541 | FAM13C |
| ENSG00000140067 | FAM181A |
| ENSG00000179813 | FAM216B |
| ENSG00000078098 | FAP |
| ENSG00000165323 | FAT3 |
| ENSG00000128610 | FEZF1 |
| ENSG00000171564 | FGB |
| ENSG00000114279 | FGF12 |
| ENSG00000171557 | FGG |
| ENSG00000094963 | FMO2 |
| ENSG00000007933 | FMO3 |
| ENSG00000110195 | FOLR1 |
| ENSG00000109101 | FOXN1 |
| ENSG00000138759 | FRAS1 |
| ENSG00000070601 | FRMPD1 |
| ENSG00000170820 | FSHR |
| ENSG00000168843 | FSTL5 |
| ENSG00000187889 | FYB2 |
| ENSG00000131482 | G6PC1 |
| ENSG00000022355 | GABRA1 |
| ENSG00000109158 | GABRA4 |
| ENSG00000186297 | GABRA5 |
| ENSG00000145864 | GABRB2 |
| ENSG00000182256 | GABRG3 |
| ENSG00000094755 | GABRP |
| ENSG00000136750 | GAD2 |
| ENSG00000144644 | GADL1 |
| ENSG00000144278 | GALNT13 |
| ENSG00000100626 | GALNT16 |
| ENSG00000106648 | GALNTL5 |
| ENSG00000135248 | GARIN1B |
| ENSG00000136574 | GATA4 |
| ENSG00000145321 | GC |
| ENSG00000084734 | GCKR |
| ENSG00000125965 | GDF5 |
| ENSG00000168621 | GDNF |
| ENSG00000178795 | GDPD4 |
| ENSG00000131095 | GFAP |
| ENSG00000146013 | GFRA3 |
| ENSG00000169605 | GKN1 |
| ENSG00000183607 | GKN2 |
| ENSG00000186417 | GLDN |
| ENSG00000180481 | GLIPR1L2 |
| ENSG00000065325 | GLP2R |
| ENSG00000145451 | GLRA3 |
| ENSG00000149124 | GLYAT |
| ENSG00000205835 | GMNC |
| ENSG00000114349 | GNAT1 |
| ENSG00000127928 | GNGT1 |
| ENSG00000169347 | GP2 |
| ENSG00000115290 | GRB14 |
| ENSG00000182771 | GRID1 |
| ENSG00000152208 | GRID2 |
| ENSG00000152822 | GRM1 |
| ENSG00000168959 | GRM5 |
| ENSG00000113262 | GRM6 |
| ENSG00000196277 | GRM7 |
| ENSG00000179603 | GRM8 |
| ENSG00000134443 | GRP |
| ENSG00000147697 | GSDMC |
| ENSG00000174156 | GSTA3 |
| ENSG00000048545 | GUCA1A |
| ENSG00000170180 | GYPA |
| ENSG00000145681 | HAPLN1 |
| ENSG00000002746 | HECW1 |
| ENSG00000010282 | HHATL |
| ENSG00000180806 | HOXC9 |
| ENSG00000113905 | HRG |
| ENSG00000203857 | HSD3B1 |
| ENSG00000203859 | HSD3B2 |
| ENSG00000126550 | HTN1 |
| ENSG00000159217 | IGF2BP1 |
| ENSG00000216588 | IGSF23 |
| ENSG00000144730 | IL17RD |
| ENSG00000136688 | IL36G |
| ENSG00000136695 | IL36RN |
| ENSG00000254647 | INS |
| ENSG00000205363 | INSYN1 |
| ENSG00000161896 | IP6K3 |
| ENSG00000184345 | IQCF2 |
| ENSG00000229972 | IQCF3 |
| ENSG00000177508 | IRX3 |
| ENSG00000016082 | ISL1 |
| ENSG00000137809 | ITGA11 |
| ENSG00000205442 | IZUMO3 |
| ENSG00000143473 | KCNH1 |
| ENSG00000140015 | KCNH5 |
| ENSG00000173826 | KCNH6 |
| ENSG00000184611 | KCNH7 |
| ENSG00000153822 | KCNJ16 |
| ENSG00000120457 | KCNJ5 |
| ENSG00000197584 | KCNMB2 |
| ENSG00000215262 | KCNU1 |
| ENSG00000164794 | KCNV1 |
| ENSG00000183775 | KCTD16 |
| ENSG00000182329 | KIAA2012 |
| ENSG00000170498 | KISS1 |
| ENSG00000162873 | KLHDC8A |
| ENSG00000150361 | KLHL1 |
| ENSG00000167757 | KLK11 |
| ENSG00000167759 | KLK13 |
| ENSG00000167751 | KLK2 |
| ENSG00000167749 | KLK4 |
| ENSG00000167754 | KLK5 |
| ENSG00000167755 | KLK6 |
| ENSG00000169035 | KLK7 |
| ENSG00000213022 | KLK9 |
| ENSG00000186847 | KRT14 |
| ENSG00000186832 | KRT16 |
| ENSG00000128422 | KRT17 |
| ENSG00000213424 | KRT222 |
| ENSG00000171446 | KRT27 |
| ENSG00000196859 | KRT39 |
| ENSG00000170477 | KRT4 |
| ENSG00000170465 | KRT6C |
| ENSG00000185640 | KRT79 |
| ENSG00000167767 | KRT80 |
| ENSG00000240871 | KRTAP4-7 |
| ENSG00000204880 | KRTAP4-8 |
| ENSG00000212722 | KRTAP4-9 |
| ENSG00000187272 | KRTAP9-8 |
| ENSG00000135413 | LACRT |
| ENSG00000101680 | LAMA1 |
| ENSG00000197084 | LCE1C |
| ENSG00000160349 | LCN1 |
| ENSG00000203985 | LDLRAD1 |
| ENSG00000145826 | LECT2 |
| ENSG00000186007 | LEMD1 |
| ENSG00000168481 | LGI3 |
| ENSG00000146166 | LGSN |
| ENSG00000187416 | LHFPL3 |
| ENSG00000106689 | LHX2 |
| ENSG00000143355 | LHX9 |
| ENSG00000253314 | LINC00293 |
| ENSG00000167459 | LINC00905 |
| ENSG00000168367 | LINC00917 |
| ENSG00000258710 | LINC01193 |
| ENSG00000205786 | LINC01531 |
| ENSG00000186960 | LINC01551 |
| ENSG00000182333 | LIPF |
| ENSG00000152936 | LMNTD1 |
| ENSG00000187185 | LOC388282 |
| ENSG00000233539 | LOC730338 |
| ENSG00000165379 | LRFN5 |
| ENSG00000081479 | LRP2 |
| ENSG00000172061 | LRRC15 |
| ENSG00000160838 | LRRC71 |
| ENSG00000157093 | LYZL4 |
| ENSG00000105695 | MAG |
| ENSG00000176601 | MAP3K19 |
| ENSG00000171533 | MAP6 |
| ENSG00000109339 | MAPK10 |
| ENSG00000151224 | MAT1A |
| ENSG00000124159 | MATN4 |
| ENSG00000234602 | MCIDAS |
| ENSG00000145794 | MEGF10 |
| ENSG00000182050 | MGAT4C |
| ENSG00000205301 | MGAT4D |
| ENSG00000254349 | MIR2052HG |
| ENSG00000146147 | MLIP |
| ENSG00000114487 | MORC1 |
| ENSG00000186732 | MPPED1 |
| ENSG00000236301 | MRGPRG-AS1 |
| ENSG00000134042 | MRO |
| ENSG00000171495 | MROH2B |
| ENSG00000166961 | MS4A15 |
| ENSG00000138823 | MTTP |
| ENSG00000169550 | MUC15 |
| ENSG00000205592 | MUC19 |
| ENSG00000172551 | MUCL1 |
| ENSG00000168631 | MUCL3 |
| ENSG00000221986 | MYBPHL |
| ENSG00000125414 | MYH2 |
| ENSG00000168530 | MYL1 |
| ENSG00000101306 | MYLK2 |
| ENSG00000187616 | MYMK |
| ENSG00000071909 | MYO3B |
| ENSG00000138347 | MYPN |
| ENSG00000186487 | MYT1L |
| ENSG00000111704 | NANOG |
| ENSG00000130287 | NCAN |
| ENSG00000176771 | NCKAP5 |
| ENSG00000184454 | NCMAP |
| ENSG00000164600 | NEUROD6 |
| ENSG00000101198 | NKAIN4 |
| ENSG00000136352 | NKX2-1 |
| ENSG00000183072 | NKX2-5 |
| ENSG00000182261 | NLRP10 |
| ENSG00000179873 | NLRP11 |
| ENSG00000160505 | NLRP4 |
| ENSG00000157064 | NMNAT2 |
| ENSG00000189332 | None |
| ENSG00000229415 | None |
| ENSG00000150526 | None |
| ENSG00000224689 | None |
| ENSG00000233611 | None |
| ENSG00000206532 | None |
| ENSG00000203690 | None |
| ENSG00000255085 | None |
| ENSG00000243444 | None |
| ENSG00000255346 | NOX5 |
| ENSG00000187258 | NPSR1 |
| ENSG00000122585 | NPY |
| ENSG00000164128 | NPY1R |
| ENSG00000197893 | NRAP |
| ENSG00000158458 | NRG2 |
| ENSG00000185737 | NRG3 |
| ENSG00000152954 | NRSN1 |
| ENSG00000185652 | NTF3 |
| ENSG00000225950 | NTF4 |
| ENSG00000182667 | NTM |
| ENSG00000148053 | NTRK2 |
| ENSG00000140538 | NTRK3 |
| ENSG00000188039 | NWD1 |
| ENSG00000122136 | OBP2A |
| ENSG00000104044 | OCA2 |
| ENSG00000155087 | ODF1 |
| ENSG00000181781 | ODF3L2 |
| ENSG00000152463 | OLAH |
| ENSG00000205927 | OLIG2 |
| ENSG00000183715 | OPCML |
| ENSG00000082556 | OPRK1 |
| ENSG00000171199 | OPRPN |
| ENSG00000188770 | OPTC |
| ENSG00000167332 | OR51E2 |
| ENSG00000155719 | OTOA |
| ENSG00000115507 | OTX1 |
| ENSG00000165588 | OTX2 |
| ENSG00000225683 | PACRG-AS3 |
| ENSG00000142623 | PADI1 |
| ENSG00000122133 | PAEP |
| ENSG00000101349 | PAK5 |
| ENSG00000116183 | PAPPA2 |
| ENSG00000135903 | PAX3 |
| ENSG00000138650 | PCDH10 |
| ENSG00000204970 | PCDHA1 |
| ENSG00000113211 | PCDHB6 |
| ENSG00000125851 | PCSK2 |
| ENSG00000056487 | PHF21B |
| ENSG00000122733 | PHF24 |
| ENSG00000165443 | PHYHIPL |
| ENSG00000164093 | PITX2 |
| ENSG00000125207 | PIWIL1 |
| ENSG00000170927 | PKHD1 |
| ENSG00000205038 | PKHD1L1 |
| ENSG00000081277 | PKP1 |
| ENSG00000159337 | PLA2G4D |
| ENSG00000188089 | PLA2G4E |
| ENSG00000122194 | PLG |
| ENSG00000148123 | PLPPR1 |
| ENSG00000147588 | PMP2 |
| ENSG00000175535 | PNLIP |
| ENSG00000187021 | PNLIPRP1 |
| ENSG00000105852 | PON3 |
| ENSG00000183206 | POTEC |
| ENSG00000168333 | PPDPFL |
| ENSG00000150722 | PPP1R1C |
| ENSG00000154415 | PPP1R3A |
| ENSG00000074211 | PPP2R2C |
| ENSG00000185686 | PRAME |
| ENSG00000061455 | PRDM6 |
| ENSG00000115592 | PRKAG3 |
| ENSG00000250799 | PRODH2 |
| ENSG00000187533 | PRR27 |
| ENSG00000231924 | PSG1 |
| ENSG00000243130 | PSG11 |
| ENSG00000243137 | PSG4 |
| ENSG00000204941 | PSG5 |
| ENSG00000124467 | PSG8 |
| ENSG00000183668 | PSG9 |
| ENSG00000244694 | PTCHD4 |
| ENSG00000171016 | PYGO1 |
| ENSG00000186867 | QRFPR |
| ENSG00000152932 | RAB3C |
| ENSG00000203722 | RAET1G |
| ENSG00000155918 | RAET1L |
| ENSG00000058335 | RASGRF1 |
| ENSG00000151962 | RBM46 |
| ENSG00000124232 | RBPJL |
| ENSG00000183324 | REC114 |
| ENSG00000189056 | RELN |
| ENSG00000111783 | RFX4 |
| ENSG00000185002 | RFX6 |
| ENSG00000148604 | RGR |
| ENSG00000117152 | RGS4 |
| ENSG00000132677 | RHBG |
| ENSG00000178796 | RIIAD1 |
| ENSG00000079841 | RIMS1 |
| ENSG00000203877 | RIPPLY2 |
| ENSG00000152214 | RIT2 |
| ENSG00000173464 | RNASE11 |
| ENSG00000165799 | RNASE7 |
| ENSG00000235631 | RNF148 |
| ENSG00000132972 | RNF17 |
| ENSG00000130561 | SAG |
| ENSG00000103449 | SALL1 |
| ENSG00000189001 | SBSN |
| ENSG00000136155 | SCEL |
| ENSG00000133488 | SEC14L4 |
| ENSG00000214491 | SEC14L6 |
| ENSG00000101251 | SEL1L2 |
| ENSG00000186838 | SELENOV |
| ENSG00000153993 | SEMA3D |
| ENSG00000170381 | SEMA3E |
| ENSG00000140093 | SERPINA10 |
| ENSG00000170054 | SERPINA9 |
| ENSG00000206073 | SERPINB4 |
| ENSG00000122852 | SFTPA1 |
| ENSG00000168484 | SFTPC |
| ENSG00000189410 | SH2D5 |
| ENSG00000107295 | SH3GL2 |
| ENSG00000148082 | SHC3 |
| ENSG00000237515 | SHISA9 |
| ENSG00000126778 | SIX1 |
| ENSG00000100625 | SIX4 |
| ENSG00000074803 | SLC12A1 |
| ENSG00000124140 | SLC12A5 |
| ENSG00000124568 | SLC17A1 |
| ENSG00000112337 | SLC17A2 |
| ENSG00000179520 | SLC17A8 |
| ENSG00000036565 | SLC18A1 |
| ENSG00000197901 | SLC22A6 |
| ENSG00000135502 | SLC26A10P |
| ENSG00000156222 | SLC28A1 |
| ENSG00000137860 | SLC28A2 |
| ENSG00000164756 | SLC30A8 |
| ENSG00000157765 | SLC34A2 |
| ENSG00000186335 | SLC36A2 |
| ENSG00000164175 | SLC45A2 |
| ENSG00000148942 | SLC5A12 |
| ENSG00000174899 | SLC66A1L |
| ENSG00000165970 | SLC6A5 |
| ENSG00000013293 | SLC7A14 |
| ENSG00000180251 | SLC9A4 |
| ENSG00000162753 | SLC9C2 |
| ENSG00000084453 | SLCO1A2 |
| ENSG00000111700 | SLCO1B3 |
| ENSG00000139155 | SLCO1C1 |
| ENSG00000121871 | SLITRK3 |
| ENSG00000170290 | SLN |
| ENSG00000171201 | SMR3B |
| ENSG00000188176 | SMTNL2 |
| ENSG00000115593 | SMYD1 |
| ENSG00000065609 | SNAP91 |
| ENSG00000074317 | SNCB |
| ENSG00000188817 | SNTN |
| ENSG00000174226 | SNX31 |
| ENSG00000141316 | SPACA3 |
| ENSG00000150628 | SPATA4 |
| ENSG00000171722 | SPATA46 |
| ENSG00000249481 | SPATS1 |
| ENSG00000178172 | SPINK6 |
| ENSG00000177679 | SRRM3 |
| ENSG00000136011 | STAB2 |
| ENSG00000145087 | STXBP5L |
| ENSG00000198203 | SULT1C2 |
| ENSG00000109193 | SULT1E1 |
| ENSG00000163630 | SYNPR |
| ENSG00000170743 | SYT9 |
| ENSG00000163377 | TAFA4 |
| ENSG00000198650 | TAT |
| ENSG00000136535 | TBR1 |
| ENSG00000092607 | TBX15 |
| ENSG00000164458 | TBXT |
| ENSG00000163792 | TCF23 |
| ENSG00000159450 | TCHH |
| ENSG00000241186 | TDGF1 |
| ENSG00000162782 | TDRD5 |
| ENSG00000205678 | TECRL |
| ENSG00000218336 | TENM3 |
| ENSG00000167014 | TERB2 |
| ENSG00000133863 | TEX15 |
| ENSG00000185264 | TEX33 |
| ENSG00000175018 | TEX36 |
| ENSG00000257987 | TEX49 |
| ENSG00000137203 | TFAP2A |
| ENSG00000087510 | TFAP2C |
| ENSG00000105825 | TFPI2 |
| ENSG00000092969 | TGFB2 |
| ENSG00000125780 | TGM3 |
| ENSG00000180176 | TH |
| ENSG00000095587 | TLL2 |
| ENSG00000166069 | TMCO5A |
| ENSG00000185332 | TMEM105 |
| ENSG00000173452 | TMEM196 |
| ENSG00000186329 | TMEM212 |
| ENSG00000198326 | TMEM239 |
| ENSG00000196932 | TMEM26 |
| ENSG00000153802 | TMPRSS11D |
| ENSG00000186452 | TMPRSS12 |
| ENSG00000159173 | TNNI1 |
| ENSG00000118194 | TNNT2 |
| ENSG00000120471 | TP53AIP1 |
| ENSG00000170777 | TPD52L3 |
| ENSG00000115705 | TPO |
| ENSG00000180438 | TPRXL |
| ENSG00000186439 | TRDN |
| ENSG00000137699 | TRIM29 |
| ENSG00000147573 | TRIM55 |
| ENSG00000177238 | TRIM72 |
| ENSG00000133107 | TRPC4 |
| ENSG00000204296 | TSBP1 |
| ENSG00000198033 | TUBA3C |
| ENSG00000112041 | TULP1 |
| ENSG00000122691 | TWIST1 |
| ENSG00000077498 | TYR |
| ENSG00000107165 | TYRP1 |
| ENSG00000156096 | UGT2B4 |
| ENSG00000137766 | UNC13C |
| ENSG00000156687 | UNC5D |
| ENSG00000144406 | UNC80 |
| ENSG00000128564 | VGF |
| ENSG00000170162 | VGLL2 |
| ENSG00000206538 | VGLL3 |
| ENSG00000170419 | VSTM2A |
| ENSG00000158816 | VWA5B1 |
| ENSG00000174453 | VWC2L |
| ENSG00000136918 | WDR38 |
| ENSG00000171804 | WDR87 |
| ENSG00000152977 | ZIC1 |
| ENSG00000144331 | ZNF385B |
| ENSG00000042813 | ZPBP |
| ENSG00000186075 | ZPBP2 |
| ENSG00000163012 | ZSWIM2 |

TABLE 2
Gene Panel: Training Genes (on)

| ON Training Gene Ensembl ID | Gene Symbol |
|---|---|
| ENSG00000087884 | AAMDC |
| ENSG00000165029 | ABCA1 |
| ENSG00000106077 | ABHD11 |
| ENSG00000163322 | ABRAXAS1 |
| ENSG00000101473 | ACOT8 |
| ENSG00000075624 | ACTB |
| ENSG00000138071 | ACTR2 |
| ENSG00000075089 | ACTR6 |
| ENSG00000151694 | ADAM17 |
| ENSG00000151651 | ADAM8 |
| ENSG00000087274 | ADD1 |
| ENSG00000159346 | ADIPOR1 |
| ENSG00000130706 | ADRM1 |
| ENSG00000171763 | AFG2B |
| ENSG00000106351 | AGFG2 |
| ENSG00000126705 | AHDC1 |
| ENSG00000204472 | AIF1 |
| ENSG00000147853 | AK3 |
| ENSG00000127914 | AKAP9 |
| ENSG00000105186 | ANKRD27 |
| ENSG00000185101 | ANO9 |
| ENSG00000182718 | ANXA2 |
| ENSG00000177721 | ANXA2R |
| ENSG00000164111 | ANXA5 |
| ENSG00000138660 | AP1AR |
| ENSG00000242802 | AP5Z1 |
| ENSG00000134982 | APC |
| ENSG00000138613 | APH1B |
| ENSG00000047365 | ARAP2 |
| ENSG00000134884 | ARGLU1 |
| ENSG00000159314 | ARHGAP27 |
| ENSG00000141522 | ARHGDIA |
| ENSG00000182196 | ARL6IP4 |
| ENSG00000197070 | ARRDC1 |
| ENSG00000065802 | ASB1 |
| ENSG00000148331 | ASB6 |
| ENSG00000115966 | ATF2 |
| ENSG00000085978 | ATG16L1 |
| ENSG00000197548 | ATG7 |
| ENSG00000069849 | ATP1B3 |
| ENSG00000156411 | ATP5MJ |
| ENSG00000154723 | ATP5PF |
| ENSG00000113732 | ATP6V0E1 |
| ENSG00000171953 | ATPAF2 |
| ENSG00000121578 | B4GALT4 |
| ENSG00000166170 | BAG5 |
| ENSG00000175866 | BAIAP2 |
| ENSG00000176788 | BASP1 |
| ENSG00000174483 | BBS1 |
| ENSG00000110987 | BCL7A |
| ENSG00000161267 | BDH1 |
| ENSG00000145919 | BOD1 |
| ENSG00000095564 | BTAF1 |
| ENSG00000110696 | C11orf58 |
| ENSG00000167747 | C19orf48P |
| ENSG00000241852 | C8orf58 |
| ENSG00000129007 | CALML4 |
| ENSG00000179218 | CALR |
| ENSG00000014216 | CAPN1 |
| ENSG00000130940 | CASZ1 |
| ENSG00000105879 | CBLL1 |
| ENSG00000100211 | CBY1 |
| ENSG00000158941 | CCAR2 |
| ENSG00000105173 | CCNE1 |
| ENSG00000138764 | CCNG2 |
| ENSG00000108100 | CCNY |
| ENSG00000163249 | CCNYL1 |
| ENSG00000139193 | CD27 |
| ENSG00000026508 | CD44 |
| ENSG00000135404 | CD63 |
| ENSG00000173762 | CD7 |
| ENSG00000105401 | CDC37 |
| ENSG00000170779 | CDCA4 |
| ENSG00000144354 | CDCA7 |
| ENSG00000006837 | CDKL3 |
| ENSG00000239704 | CDRT4 |
| ENSG00000101290 | CDS2 |
| ENSG00000123219 | CENPK |
| ENSG00000138092 | CENPO |
| ENSG00000121289 | CEP89 |
| ENSG00000172757 | CFL1 |
| ENSG00000111642 | CHD4 |
| ENSG00000177830 | CHID1 |
| ENSG00000100288 | CHKB |
| ENSG00000176108 | CHMP6 |
| ENSG00000147457 | CHMP7 |
| ENSG00000136213 | CHST12 |
| ENSG00000131873 | CHSY1 |
| ENSG00000164442 | CITED2 |
| ENSG00000254788 | CKLF-CMTM1 |
| ENSG00000013441 | CLK1 |
| ENSG00000179335 | CLK3 |
| ENSG00000187118 | CMC1 |
| ENSG00000180917 | CMTR2 |
| ENSG00000125107 | CNOT1 |
| ENSG00000138767 | CNOT6L |
| ENSG00000166997 | CNPY4 |
| ENSG00000068120 | COASY |
| ENSG00000164597 | COG5 |
| ENSG00000188243 | COMMD6 |
| ENSG00000198612 | COPS8 |
| ENSG00000163050 | COQ8A |
| ENSG00000133983 | COX16 |
| ENSG00000138495 | COX17 |
| ENSG00000135940 | COX5B |
| ENSG00000108582 | CPD |
| ENSG00000140848 | CPNE2 |
| ENSG00000213145 | CRIP1 |
| ENSG00000112297 | CRYBG1 |
| ENSG00000113712 | CSNK1A1 |
| ENSG00000159176 | CSRP1 |
| ENSG00000178585 | CTNNBIP1 |
| ENSG00000171793 | CTPS1 |
| ENSG00000064601 | CTSA |
| ENSG00000109861 | CTSC |
| ENSG00000174080 | CTSF |
| ENSG00000119929 | CUTC |
| ENSG00000152404 | CWF19L2 |
| ENSG00000153310 | CYRIB |
| ENSG00000162384 | CZIB |
| ENSG00000183283 | DAZAP2 |
| ENSG00000137992 | DBT |
| ENSG00000156136 | DCK |
| ENSG00000129187 | DCTD |
| ENSG00000132912 | DCTN4 |
| ENSG00000079785 | DDX1 |
| ENSG00000108654 | DDX5 |
| ENSG00000110367 | DDX6 |
| ENSG00000174839 | DENND6A |
| ENSG00000108771 | DHX58 |
| ENSG00000086189 | DIMT1 |
| ENSG00000150768 | DLAT |
| ENSG00000105993 | DNAJB6 |
| ENSG00000087470 | DNM1L |
| ENSG00000130816 | DNMT1 |
| ENSG00000167130 | DOLPP1 |
| ENSG00000159147 | DONSON |
| ENSG00000083097 | DOP1A |
| ENSG00000254986 | DPP3 |
| ENSG00000144635 | DYNC1LI1 |
| ENSG00000013016 | EHD3 |
| ENSG00000119718 | EIF2B2 |
| ENSG00000130811 | EIF3G |
| ENSG00000156976 | EIF4A2 |
| ENSG00000184708 | EIF4ENIF1 |
| ENSG00000163832 | ELP6 |
| ENSG00000170571 | EMB |
| ENSG00000125037 | EMC3 |
| ENSG00000165521 | EML5 |
| ENSG00000108515 | ENO3 |
| ENSG00000099219 | ERMP1 |
| ENSG00000141446 | ESCO1 |
| ENSG00000089048 | ESF1 |
| ENSG00000164002 | EXO5 |
| ENSG00000107371 | EXOSC3 |
| ENSG00000151348 | EXT2 |
| ENSG00000055147 | FAM114A2 |
| ENSG00000054965 | FAM168A |
| ENSG00000228300 | FAM174C |
| ENSG00000167930 | FAM234A |
| ENSG00000105058 | FAM32A |
| ENSG00000120709 | FAM53C |
| ENSG00000188878 | FBF1 |
| ENSG00000156860 | FBRS |
| ENSG00000151876 | FBXO4 |
| ENSG00000157353 | FCSK |
| ENSG00000151422 | FER |
| ENSG00000111790 | FGFR1OP2 |
| ENSG00000132436 | FIGNL1 |
| ENSG00000145216 | FIP1L1 |
| ENSG00000000460 | FIRRM |
| ENSG00000100442 | FKBP3 |
| ENSG00000065970 | FOXJ2 |
| ENSG00000114416 | FXR1 |
| ENSG00000143891 | GALM |
| ENSG00000255284 | GATD1-DT |
| ENSG00000174500 | GCSAM |
| ENSG00000198356 | GET3 |
| ENSG00000152904 | GGPS1 |
| ENSG00000106560 | GIMAP2 |
| ENSG00000055211 | GINM1 |
| ENSG00000147536 | GINS4 |
| ENSG00000139278 | GLIPR1 |
| ENSG00000122694 | GLIPR2 |
| ENSG00000197045 | GMFB |
| ENSG00000112312 | GMNN |
| ENSG00000090581 | GNPTG |
| ENSG00000166734 | GOLM2 |
| ENSG00000076650 | GPATCH1 |
| ENSG00000111231 | GPN3 |
| ENSG00000132522 | GPS2 |
| ENSG00000121957 | GPSM2 |
| ENSG00000030582 | GRN |
| ENSG00000109519 | GRPEL1 |
| ENSG00000084207 | GSTP1 |
| ENSG00000121964 | GTDC1 |
| ENSG00000165417 | GTF2A1 |
| ENSG00000153767 | GTF2E1 |
| ENSG00000158406 | H4C8 |
| ENSG00000049239 | H6PD |
| ENSG00000143575 | HAX1 |
| ENSG00000105856 | HBP1 |
| ENSG00000206344 | HCG27 |
| ENSG00000008869 | HEATR5B |
| ENSG00000013583 | HEBP1 |
| ENSG00000092148 | HECTD1 |
| ENSG00000119969 | HELLS |
| ENSG00000130589 | HELZ2 |
| ENSG00000197568 | HHLA3 |
| ENSG00000234745 | HLA-B |
| ENSG00000241106 | HLA-DOB |
| ENSG00000112972 | HMGCS1 |
| ENSG00000221887 | HMSD |
| ENSG00000099783 | HNRNPM |
| ENSG00000125944 | HNRNPR |
| ENSG00000134709 | HOOK1 |
| ENSG00000127483 | HP1BP3 |
| ENSG00000133835 | HSD17B4 |
| ENSG00000101052 | IFT52 |
| ENSG00000096872 | IFT74 |
| ENSG00000032742 | IFT88 |
| ENSG00000132740 | IGHMBP2 |
| ENSG00000177663 | IL17RA |
| ENSG00000196083 | IL1RAP |
| ENSG00000134352 | IL6ST |
| ENSG00000105135 | ILVBL |
| ENSG00000197429 | IPP |
| ENSG00000145703 | IQGAP2 |
| ENSG00000142856 | ITGB3BP |
| ENSG00000086544 | ITPKC |
| ENSG00000050130 | JKAMP |
| ENSG00000168970 | JMJD7-PLA2G4B |
| ENSG00000116852 | KIF21B |
| ENSG00000068796 | KIF2A |
| ENSG00000131437 | KIF3A |
| ENSG00000127528 | KLF2 |
| ENSG00000164344 | KLKB1 |
| ENSG00000133247 | KMT5C |
| ENSG00000198945 | L3MBTL3 |
| ENSG00000149357 | LAMTOR1 |
| ENSG00000134333 | LDHA |
| ENSG00000213625 | LEPROT |
| ENSG00000100097 | LGALS1 |
| ENSG00000187624 | LIAT1 |
| ENSG00000079435 | LIPE |
| ENSG00000131899 | LLGL1 |
| ENSG00000114988 | LMAN2L |
| ENSG00000100258 | LMF2 |
| ENSG00000235978 | LOC124906209 |
| ENSG00000102910 | LONP2 |
| ENSG00000123684 | LPGAT1 |
| ENSG00000171488 | LRRC8C |
| ENSG00000240720 | LRRD1 |
| ENSG00000148356 | LRSAM1 |
| ENSG00000106355 | LSM5 |
| ENSG00000111144 | LTA4H |
| ENSG00000213906 | LTB4R2 |
| ENSG00000198862 | LTN1 |
| ENSG00000160932 | LY6E |
| ENSG00000143353 | LYPLAL1 |
| ENSG00000178573 | MAF |
| ENSG00000198162 | MAN1A2 |
| ENSG00000013288 | MAN2B2 |
| ENSG00000130479 | MAP1S |
| ENSG00000104814 | MAP4K1 |
| ENSG00000088888 | MAVS |
| ENSG00000204406 | MBD5 |
| ENSG00000166987 | MBD6 |
| ENSG00000152601 | MBNL1 |
| ENSG00000180398 | MCFD2 |
| ENSG00000100297 | MCM5 |
| ENSG00000135272 | MDFIC |
| ENSG00000104973 | MED25 |
| ENSG00000141026 | MED9 |
| ENSG00000138382 | METTL5 |
| ENSG00000130382 | MLLT1 |
| ENSG00000139428 | MMAB |
| ENSG00000133422 | MORC2 |
| ENSG00000160588 | MPZL3 |
| ENSG00000170262 | MRAP |
| ENSG00000011028 | MRC2 |
| ENSG00000037757 | MRI1 |
| ENSG00000066697 | MSANTD3 |
| ENSG00000052802 | MSMO1 |
| ENSG00000187193 | MT1X |
| ENSG00000128654 | MTX2 |
| ENSG00000183486 | MX2 |
| ENSG00000173418 | NAA20 |
| ENSG00000123338 | NCKAP1L |
| ENSG00000130414 | NDUFA10 |
| ENSG00000170906 | NDUFA3 |
| ENSG00000164182 | NDUFAF2 |
| ENSG00000147684 | NDUFB9 |
| ENSG00000115286 | NDUFS7 |
| ENSG00000139350 | NEDD1 |
| ENSG00000204386 | NEU1 |
| ENSG00000162139 | NEU3 |
| ENSG00000104825 | NFKBIB |
| ENSG00000001167 | NFYA |
| ENSG00000120837 | NFYB |
| ENSG00000129460 | NGDN |
| ENSG00000145029 | NICN1 |
| ENSG00000001461 | NIPAL3 |
| ENSG00000010322 | NISCH |
| ENSG00000158793 | NIT1 |
| ENSG00000225921 | NOL7 |
| ENSG00000258465 | None |
| ENSG00000203709 | None |
| ENSG00000255062 | None |
| ENSG00000259066 | None |
| ENSG00000214176 | None |
| ENSG00000103148 | NPRL3 |
| ENSG00000113580 | NR3C1 |
| ENSG00000078618 | NRDC |
| ENSG00000106459 | NRF1 |
| ENSG00000129535 | NRL |
| ENSG00000174004 | NRROS |
| ENSG00000073969 | NSF |
| ENSG00000013374 | NUB1 |
| ENSG00000155561 | NUP205 |
| ENSG00000125450 | NUP85 |
| ENSG00000198408 | OGA |
| ENSG00000138430 | OLA1 |
| ENSG00000198836 | OPA1 |
| ENSG00000110048 | OSBP |
| ENSG00000081087 | OSTM1 |
| ENSG00000101104 | PABPC1L |
| ENSG00000072415 | PALS1 |
| ENSG00000102981 | PARD6A |
| ENSG00000140694 | PARN |
| ENSG00000157212 | PAXIP1 |
| ENSG00000081154 | PCNP |
| ENSG00000197329 | PELI1 |
| ENSG00000049246 | PER3 |
| ENSG00000229833 | PET100 |
| ENSG00000143256 | PFDN2 |
| ENSG00000101132 | PFDN4 |
| ENSG00000123349 | PFDN5 |
| ENSG00000170525 | PFKFB3 |
| ENSG00000141959 | PFKL |
| ENSG00000152556 | PFKM |
| ENSG00000161395 | PGAP3 |
| ENSG00000013375 | PGM3 |
| ENSG00000087157 | PGS1 |
| ENSG00000106443 | PHF14 |
| ENSG00000040199 | PHLPP2 |
| ENSG00000135845 | PIGC |
| ENSG00000141506 | PIK3R5 |
| ENSG00000143398 | PIP5K1A |
| ENSG00000180957 | PITPNB |
| ENSG00000243708 | PLA2G4B |
| ENSG00000011422 | PLAUR |
| ENSG00000151176 | PLBD2 |
| ENSG00000115956 | PLEK |
| ENSG00000145632 | PLK2 |
| ENSG00000115946 | PNO1 |
| ENSG00000070501 | POLB |
| ENSG00000100227 | POLDIP3 |
| ENSG00000102978 | POLR2C |
| ENSG00000085998 | POMGNT1 |
| ENSG00000166794 | PPIB |
| ENSG00000170836 | PPM1D |
| ENSG00000115241 | PPM1G |
| ENSG00000163590 | PPM1L |
| ENSG00000172531 | PPP1CA |
| ENSG00000077157 | PPP1R12B |
| ENSG00000087074 | PPP1R15A |
| ENSG00000115685 | PPP1R7 |
| ENSG00000113575 | PPP2CA |
| ENSG00000119414 | PPP6C |
| ENSG00000148840 | PPRC1 |
| ENSG00000113593 | PPWD1 |
| ENSG00000163558 | PRKCI |
| ENSG00000110107 | PRPF19 |
| ENSG00000134748 | PRPF38A |
| ENSG00000185246 | PRPF39 |
| ENSG00000143363 | PRUNE1 |
| ENSG00000205155 | PSENEN |
| ENSG00000142507 | PSMB6 |
| ENSG00000136930 | PSMB7 |
| ENSG00000161057 | PSMC2 |
| ENSG00000159352 | PSMD4 |
| ENSG00000172775 | PSME3IP1 |
| ENSG00000165983 | PTER |
| ENSG00000173482 | PTPRM |
| ENSG00000172053 | QARS1 |
| ENSG00000198218 | QRICH1 |
| ENSG00000213339 | QTRT1 |
| ENSG00000198858 | R3HDM4 |
| ENSG00000185236 | RAB11B |
| ENSG00000138069 | RAB1A |
| ENSG00000080371 | RAB21 |
| ENSG00000157869 | RAB28 |
| ENSG00000182185 | RAD51B |
| ENSG00000099901 | RANBP1 |
| ENSG00000108961 | RANGRF |
| ENSG00000155903 | RASA2 |
| ENSG00000117222 | RBBP5 |
| ENSG00000185272 | RBM11 |
| ENSG00000086589 | RBM22 |
| ENSG00000106344 | RBM28 |
| ENSG00000131051 | RBM39 |
| ENSG00000126254 | RBM42 |
| ENSG00000155636 | RBM45 |
| ENSG00000139194 | RBP5 |
| ENSG00000168214 | RBPJ |
| ENSG00000159200 | RCAN1 |
| ENSG00000117602 | RCAN3 |
| ENSG00000115255 | REEP6 |
| ENSG00000164068 | RNF123 |
| ENSG00000118518 | RNF146 |
| ENSG00000163162 | RNF149 |
| ENSG00000163961 | RNF168 |
| ENSG00000011275 | RNF216 |
| ENSG00000063978 | RNF4 |
| ENSG00000101654 | RNMT |
| ENSG00000156990 | RPUSD3 |
| ENSG00000103472 | RRN3P2 |
| ENSG00000215012 | RTL10 |
| ENSG00000133318 | RTN3 |
| ENSG00000020633 | RUNX3 |
| ENSG00000111832 | RWDD1 |
| ENSG00000167100 | SAMD14 |
| ENSG00000152700 | SAR1B |
| ENSG00000075856 | SART3 |
| ENSG00000139218 | SCAF11 |
| ENSG00000184178 | SCFD2 |
| ENSG00000116171 | SCP2 |
| ENSG00000166562 | SEC11C |
| ENSG00000157020 | SEC13 |
| ENSG00000071537 | SEL1L |
| ENSG00000211450 | SELENOH |
| ENSG00000131871 | SELENOS |
| ENSG00000188404 | SELL |
| ENSG00000185033 | SEMA4B |
| ENSG00000129158 | SERGEF |
| ENSG00000097033 | SH3GLB1 |
| ENSG00000113558 | SKP1 |
| ENSG00000089057 | SLC23A2 |
| ENSG00000114120 | SLC25A36 |
| ENSG00000147454 | SLC25A37 |
| ENSG00000110660 | SLC35F2 |
| ENSG00000180773 | SLC36A4 |
| ENSG00000138449 | SLC40A1 |
| ENSG00000133065 | SLC41A1 |
| ENSG00000169241 | SLC50A1 |
| ENSG00000103257 | SLC7A5 |
| ENSG00000119705 | SLIRP |
| ENSG00000137776 | SLTM |
| ENSG00000073584 | SMARCE1 |
| ENSG00000135587 | SMPD2 |
| ENSG00000104852 | SNRNP70 |
| ENSG00000131876 | SNRPA1 |
| ENSG00000167088 | SNRPD1 |
| ENSG00000139343 | SNRPF |
| ENSG00000100603 | SNW1 |
| ENSG00000167208 | SNX20 |
| ENSG00000124104 | SNX21 |
| ENSG00000148158 | SNX30 |
| ENSG00000129515 | SNX6 |
| ENSG00000140263 | SORD |
| ENSG00000066336 | SPI1 |
| ENSG00000157837 | SPPL3 |
| ENSG00000198911 | SREBF2 |
| ENSG00000151304 | SRFBP1 |
| ENSG00000096063 | SRPK1 |
| ENSG00000116754 | SRSF11 |
| ENSG00000007341 | ST7L |
| ENSG00000115415 | STAT1 |
| ENSG00000143093 | STRIP1 |
| ENSG00000111450 | STX2 |
| ENSG00000079950 | STX7 |
| ENSG00000113387 | SUB1 |
| ENSG00000116030 | SUMO1 |
| ENSG00000184900 | SUMO3 |
| ENSG00000213246 | SUPT4H1 |
| ENSG00000198168 | SVIP |
| ENSG00000159082 | SYNJ1 |
| ENSG00000139546 | TARBP2 |
| ENSG00000120948 | TARDBP |
| ENSG00000157014 | TATDN2 |
| ENSG00000106052 | TAX1BP1 |
| ENSG00000099992 | TBC1D10A |
| ENSG00000136111 | TBC1D4 |
| ENSG00000131374 | TBC1D5 |
| ENSG00000183735 | TBK1 |
| ENSG00000106638 | TBL2 |
| ENSG00000137310 | TCF19 |
| ENSG00000110719 | TCIRG1 |
| ENSG00000070814 | TCOF1 |
| ENSG00000168769 | TET2 |
| ENSG00000116001 | TIA1 |
| ENSG00000140993 | TIGD7 |
| ENSG00000075336 | TIMM21 |
| ENSG00000104980 | TIMM44 |
| ENSG00000119139 | TJP2 |
| ENSG00000077147 | TM9SF3 |
| ENSG00000101337 | TM9SF4 |
| ENSG00000113119 | TMCO6 |
| ENSG00000251201 | TMED7-TICAM2 |
| ENSG00000171202 | TMEM126A |
| ENSG00000171204 | TMEM126B |
| ENSG00000131634 | TMEM204 |
| ENSG00000146842 | TMEM209 |
| ENSG00000187824 | TMEM220 |
| ENSG00000134825 | TMEM258 |
| ENSG00000151881 | TMEM267 |
| ENSG00000169964 | TMEM42 |
| ENSG00000103978 | TMEM87A |
| ENSG00000185215 | TNFAIP2 |
| ENSG00000196683 | TOMM7 |
| ENSG00000196511 | TPK1 |
| ENSG00000082512 | TRAF5 |
| ENSG00000135148 | TRAFD1 |
| ENSG00000141569 | TRIM65 |
| ENSG00000123144 | TRIR |
| ENSG00000066651 | TRMT11 |
| ENSG00000100416 | TRMU |
| ENSG00000100991 | TRPC4AP |
| ENSG00000103197 | TSC2 |
| ENSG00000167721 | TSR1 |
| ENSG00000128881 | TTBK2 |
| ENSG00000149292 | TTC12 |
| ENSG00000163728 | TTC14 |
| ENSG00000100271 | TTLL1 |
| ENSG00000167553 | TUBA1C |
| ENSG00000178952 | TUFM |
| ENSG00000110344 | UBE4A |
| ENSG00000175567 | UCP2 |
| ENSG00000120686 | UFM1 |
| ENSG00000109103 | UNC119 |
| ENSG00000124602 | UNC5CL |
| ENSG00000127540 | UQCR11 |
| ENSG00000164405 | UQCRQ |
| ENSG00000126088 | UROD |
| ENSG00000123552 | USP45 |
| ENSG00000164663 | USP49 |
| ENSG00000124164 | VAPB |
| ENSG00000129003 | VPS13C |
| ENSG00000104142 | VPS18 |
| ENSG00000131475 | VPS25 |
| ENSG00000136100 | VPS36 |
| ENSG00000004766 | VPS50 |
| ENSG00000141252 | VPS53 |
| ENSG00000100568 | VTI1B |
| ENSG00000139668 | WDFY2 |
| ENSG00000184575 | XPOT |
| ENSG00000066422 | ZBTB11 |
| ENSG00000109906 | ZBTB16 |
| ENSG00000179456 | ZBTB18 |
| ENSG00000174282 | ZBTB4 |
| ENSG00000160446 | ZDHHC12 |
| ENSG00000177054 | ZDHHC13 |
| ENSG00000186908 | ZDHHC17 |
| ENSG00000156599 | ZDHHC5 |
| ENSG00000148516 | ZEB1 |
| ENSG00000160445 | ZER1 |
| ENSG00000158552 | ZFAND2B |
| ENSG00000066827 | ZFAT |
| ENSG00000152518 | ZFP36L2 |
| ENSG00000187954 | ZFTRAF1 |
| ENSG00000164631 | ZNF12 |
| ENSG00000166478 | ZNF143 |
| ENSG00000160321 | ZNF208 |
| ENSG00000172466 | ZNF24 |
| ENSG00000198105 | ZNF248 |
| ENSG00000137871 | ZNF280D |
| ENSG00000170265 | ZNF282 |
| ENSG00000145908 | ZNF300 |
| ENSG00000131116 | ZNF428 |
| ENSG00000197647 | ZNF433 |
| ENSG00000183621 | ZNF438 |
| ENSG00000197044 | ZNF441 |
| ENSG00000081665 | ZNF506 |
| ENSG00000189164 | ZNF527 |
| ENSG00000186020 | ZNF529 |
| ENSG00000204519 | ZNF551 |
| ENSG00000188321 | ZNF559 |
| ENSG00000167395 | ZNF646 |
| ENSG00000197372 | ZNF675 |
| ENSG00000181450 | ZNF678 |
| ENSG00000181135 | ZNF707 |
| ENSG00000140548 | ZNF710 |
| ENSG00000196417 | ZNF765 |
| ENSG00000175691 | ZNF77 |
| ENSG00000196456 | ZNF775 |
| ENSG00000152443 | ZNF776 |
| ENSG00000196597 | ZNF782 |
| ENSG00000198482 | ZNF808 |
| ENSG00000223547 | ZNF844 |
| ENSG00000106479 | ZNF862 |
| ENSG00000136682 | ZNG1B |
| ENSG00000197037 | ZSCAN25 |
| ENSG00000140987 | ZSCAN32 |
| ENSG00000070476 | ZXDC |

TABLE 3
Gene Panel: Cancer Panel 1

| Gene Name | Hedges' g |
|---|---|
| PLA2G2F | 1.479113632 |
| SSTR2 | 1.259250217 |
| PHLDB2 | 1.246920665 |
| PSAPL1 | 1.147912418 |
| SERPINB5 | 1.107365096 |
| AMY2A | 1.080384737 |
| LOC389199 | 1.076884032 |
| PPFIA2 | 1.061913574 |
| AGBL4 | 1.057605692 |
| BMPR1B | 1.05679972 |
| SCGB1D2 | 1.04701516 |
| C21orf58 | 1.038894657 |
| DSCR4 | 1.035225556 |
| VTCN1 | 1.030307028 |
| SCGB2A2 | 1.027227639 |
| VEPH1 | 1.023374721 |
| TMPRSS11F | 1.022785349 |
| AGR3 | 0.991225109 |
| LRRC52 | 0.990839341 |
| B3GALT2 | 0.980263125 |
| ATP2C2 | 0.972054278 |
| CDHR3 | 0.971579253 |
| SYT1 | 0.963777915 |
| ZNF492 | 0.962040195 |
| TMEM95 | 0.961920394 |
| MSRB1 | 0.958179899 |
| ADGRF1 | 0.953555597 |
| CAPN8 | 0.95240347 |
| TFAP2B | 0.951841816 |
| C11orf88 | 0.947584487 |
| MT1B | 0.947172792 |
| NKX1-2 | 0.943939887 |
| ATG9B | 0.942291227 |
| OR13J1 | 0.942223103 |
| INSC | 0.941328468 |
| BDKRB2 | 0.94019543 |
| PRKG1 | 0.939222414 |
| PWP1 | 0.936897969 |
| CXCL8 | 0.931802443 |
| TMEM116 | 0.93055595 |
| RASEF | 0.929918886 |
| TUB | 0.922005202 |
| SLC7A9 | 0.920245163 |
| ATOH7 | 0.918516102 |
| OSBPL1A | 0.915360946 |
| CECR2 | 0.912914877 |
| C10orf107 | 0.910843796 |
| ZMYND10 | 0.909366008 |
| DMP1 | 0.898958606 |
| SORBS2 | 0.896649477 |
| CALML5 | 0.896099843 |
| CHST9 | 0.894488754 |
| STX19 | 0.89140078 |
| HSD17B6 | 0.889147059 |
| TXNDC8 | 0.888369765 |
| GRHL2 | 0.888218463 |
| BTC | 0.887561792 |
| MTUS2 | 0.88614213 |
| SLC28A3 | 0.885590527 |
| LARP1 | 0.880371093 |
| RIPK4 | 0.876042172 |
| NKX2-8 | 0.875014449 |
| ATP6V1B1 | 0.871295951 |
| OTOG | 0.869168546 |
| OSBPL10 | 0.865766096 |
| NDST4 | 0.864807425 |
| MUC21 | 0.863279853 |
| IL22RA2 | 0.86252253 |
| SCNM1 | 0.858682255 |
| ARID3B | 0.856681299 |
| KRTDAP | 0.856470266 |
| QSOX1 | 0.85587722 |
| CLEC19A | 0.849586226 |
| FAM179A | 0.848582485 |
| BCL2L14 | 0.845695271 |
| LIN7A | 0.845237311 |
| B3GNT6 | 0.844911289 |
| HSPB8 | 0.844325811 |
| CP | 0.842971756 |
| C11orf52 | 0.840095948 |
| SLC39A2 | 0.836807749 |
| KRT23 | 0.832040085 |
| PROM2 | 0.831759052 |
| ABCC8 | 0.82918515 |
| SMOC2 | 0.828538667 |
| BCAS1 | 0.822712404 |
| ZNF619 | 0.822535837 |
| C16orf82 | 0.822366328 |
| SAXO1 | 0.821330477 |
| RASSF10 | 0.820454864 |
| RGS12 | 0.819725959 |
| MKRN3 | 0.816783798 |
| OR2S2 | 0.816046187 |
| IARS2 | 0.811549911 |
| CST5 | 0.811155143 |
| FAM71D | 0.810501279 |
| SEPP1 | 0.809604291 |
| C1QTNF8 | 0.809038031 |
| RAB1B | 0.808075516 |
| DNAH11 | 0.807677861 |
| SLC39A4 | 0.807043043 |
| PEX19 | 0.80392369 |
| JAKMIP1 | 0.803334017 |
| KDF1 | 0.803082402 |
| BCLAF1 | 0.802439598 |
| SDR16C5 | 0.8014299 |
| SMIM14 | 0.79953674 |
| RANBP17 | 0.799431911 |
| BEST1 | 0.798620852 |
| BRINP3 | 0.795838473 |
| CSNK1G3 | 0.795233652 |
| COL21A1 | 0.794478957 |
| LEUTX | 0.794456619 |
| SGPP2 | 0.794185606 |
| NME1 | 0.790857862 |
| DHRS7C | 0.787856241 |
| TGM7 | 0.785114213 |
| DAZL | 0.784454543 |
| OR2T27 | 0.78375321 |
| SPINT1 | 0.782690843 |
| SPC24 | 0.782043415 |
| PHF3 | 0.781401228 |
| ARHGEF38 | 0.781335837 |
| GOLT1A | 0.780315838 |
| ALX1 | 0.780215363 |
| ARG1 | 0.779716802 |
| LOC101929926 | 0.778834751 |
| CRCT1 | 0.778391598 |
| MFAP2 | 0.777758087 |
| COL9A1 | 0.775967908 |
| ZNF804B | 0.775501106 |
| IREB2 | 0.773404179 |
| STAT3 | 0.772904993 |
| PDIA6 | 0.769319454 |
| SQLE | 0.768964981 |
| PALLD | 0.76870361 |
| GPC6 | 0.76860995 |
| NAALADL2 | 0.767762495 |
| S100A16 | 0.766964261 |
| NOA1 | 0.766287307 |
| YIF1B | 0.76628538 |
| HCAR1 | 0.765490244 |
| TMEM242 | 0.764767245 |
| GALNT11 | 0.764322711 |
| FAM227B | 0.764181078 |
| SCRG1 | 0.764121165 |
| ITGA8 | 0.763073898 |
| SH3RF2 | 0.763069866 |
| TBC1D22B | 0.762648056 |
| SLIT1 | 0.762420691 |
| LOC730098 | 0.761820253 |
| SCGB3A2 | 0.759531029 |
| KDR | 0.759120018 |
| OPALIN | 0.757181261 |
| EYA2 | 0.756709865 |
| SYDE1 | 0.756290073 |
| TRAM1L1 | 0.755868019 |
| C6orf163 | 0.755798639 |
| ZNF839 | 0.754563784 |
| ACTRT2 | 0.754485211 |
| PCDHGA1 | 0.753283821 |
| CSRNP3 | 0.752578336 |
| PDE4D | 0.752080784 |
| GALR1 | 0.751966128 |
| NELFE | 0.74811493 |
| FLRT3 | 0.747608139 |
| PLIN1 | 0.74735436 |
| PLA2G2A | 0.746817545 |
| ARHGEF19 | 0.746505065 |
| B3GALT5 | 0.746280698 |
| KLHL38 | 0.746021048 |
| NECTIN4 | 0.745776097 |
| RNF183 | 0.745404572 |
| FAM101A | 0.744719934 |
| KRTAP2-2 | 0.744106239 |
| SLC16A12 | 0.743711283 |
| KRTAP2-4 | 0.742455426 |
| RAG2 | 0.741861056 |
| DSCAM | 0.741688208 |
| ENPP5 | 0.741362931 |
| SENP2 | 0.740992105 |
| ADAMTSL1 | 0.740732347 |
| CASP14 | 0.740267136 |
| PDE7A | 0.738172617 |
| DNAJC12 | 0.735910409 |
| VIT | 0.735254318 |
| ERN1 | 0.734979234 |
| IGFBP1 | 0.734260931 |
| ADAMTSL3 | 0.733884896 |
| C8orf86 | 0.733836081 |
| ELF1 | 0.733631989 |
| LAMB1 | 0.733596778 |
| FAM163B | 0.732520205 |
| KRTAP9-7 | 0.732488919 |
| PHGDH | 0.731842153 |
| ANKRD1 | 0.73107111 |
| DNAJC11 | 0.730825371 |
| DCT | 0.730789646 |
| SGCZ | 0.729623575 |
| PANX3 | 0.729313078 |
| PRPF6 | 0.726976843 |
| RPL19 | 0.72615221 |
| GJB4 | 0.724097402 |
| NRG1 | 0.721561462 |
| PLEKHD1 | 0.720262167 |
| RHOD | 0.719411068 |
| ZNF416 | 0.716048677 |
| ANGPTL5 | 0.715484253 |
| SLC26A4 | 0.715456528 |
| TMPRSS11E | 0.714906095 |
| CD3G | 0.711733186 |
| SVIL | 0.71089869 |
| SERPINA5 | 0.710658109 |
| MRPL38 | 0.710481378 |
| C10orf142 | 0.710419772 |
| ASTN1 | 0.709802032 |
| RNF43 | 0.709259692 |
| TM4SF18 | 0.709055158 |
| GYLTL1B | 0.708992425 |
| APOF | 0.707045825 |
| CENPF | 0.706686526 |
| CLDN25 | 0.706345957 |
| NCOA2 | 0.706137641 |
| RNF222 | 0.705853698 |
| HOXC6 | 0.705776615 |
| SHROOM3 | 0.705400105 |
| MIR205HG | 0.70535375 |
| KIF1A | 0.704739481 |
| NNMT | 0.704237811 |
| ACKR3 | 0.704021708 |
| ZHX3 | 0.70373115 |
| TMEM108 | 0.703195296 |
| SLC35B1 | 0.703056985 |
| PRR15L | 0.702208417 |
| CNTF | 0.701971446 |
| TRIM6-TRIM34 | 0.700839081 |
| IQCA1 | 0.700109304 |
| DMTF1 | 0.69944822 |
| MEF2B | 0.69754401 |
| CLEC3A | 0.697199238 |
| HTR1E | 0.696702883 |
| OSMR | 0.696203453 |
| WFDC6 | 0.696146391 |
| TMCO4 | 0.696100093 |
| MYO1B | 0.696066216 |
| MRPL32 | 0.694619512 |
| PLCB4 | 0.694580196 |
| CYP2E1 | 0.693497278 |
| BDKRB1 | 0.692990182 |
| PDPN | 0.692661949 |
| CNDP1 | 0.692400218 |
| CNN2 | 0.692381872 |
| KCNS3 | 0.691310117 |
| DPH3P1 | 0.690603383 |
| MAB21L3 | 0.688357139 |
| CLDN22 | 0.686952957 |
| DSC2 | 0.686821941 |
| SCN2A | 0.685049363 |
| ATP13A4 | 0.684722738 |
| NOSTRIN | 0.684622908 |
| STC2 | 0.684328866 |
| PTX3 | 0.683961362 |
| IFT27 | 0.68371629 |
| ARSB | 0.683632061 |
| CAPNS1 | 0.683318089 |
| PRAMEF20 | 0.683300855 |
| CRISP3 | 0.682963938 |
| SRBD1 | 0.68150672 |
| FXYD6 | 0.68090319 |
| PPIP5K2 | 0.680782987 |
| LOC101928093 | 0.680705086 |
| ADAMTS8 | 0.680329632 |
| TMPRSS4 | 0.679930016 |
| CAPN13 | 0.679905228 |
| BPIFB2 | 0.679856488 |
| FREM2 | 0.679016245 |
| FAM134B | 0.678690809 |
| PIK3C2G | 0.678290905 |
| HMGCS2 | 0.677950893 |
| PRSS45 | 0.677755519 |
| MAGEF1 | 0.677404391 |
| USP17L3 | 0.674411112 |
| MYT1 | 0.674009338 |
| HTR3A | 0.672300443 |
| DMRT1 | 0.671853574 |
| RAB5C | 0.671589469 |
| SLC6A9 | 0.671530157 |
| MYH14 | 0.671425924 |
| IFI27 | 0.67097674 |
| EDDM3A | 0.670944595 |
| MUC2 | 0.670450788 |
| RPS11 | 0.669434817 |
| PAIP2 | 0.669233603 |
| SLC16A4 | 0.669052569 |
| RTP5 | 0.666422751 |
| LNX1 | 0.665919304 |
| CCL22 | 0.664369818 |
| HIBADH | 0.664272038 |
| PLCD4 | 0.664266538 |
| KRI1 | 0.664078832 |
| RBM24 | 0.66333471 |
| ZNF232 | 0.662760837 |
| FGF21 | 0.662518569 |
| RPGRIP1 | 0.661641512 |
| ACBD4 | 0.661230828 |
| WFDC13 | 0.661060029 |
| CELF1 | 0.659828646 |
| HDGF | 0.659759379 |
| FUT2 | 0.659545955 |
| MAMDC2 | 0.65799203 |
| FGL1 | 0.657723031 |
| FAM184A | 0.657395512 |
| KRT9 | 0.656336095 |
| FAM83B | 0.656311295 |
| CDH16 | 0.656126929 |
| PALMD | 0.654598041 |
| SLC47A2 | 0.654117855 |
| C4orf26 | 0.653578576 |
| TRABD2A | 0.653309756 |
| WEE2 | 0.652885547 |
| TRPV3 | 0.652089342 |
| PPP1R27 | 0.652022629 |
| CNTN1 | 0.652017217 |
| FANCI | 0.651592728 |
| PIGF | 0.651384865 |
| MTMR6 | 0.65094265 |
| EPHB6 | 0.650752266 |
| ARID1B | 0.650336684 |
| LPAR3 | 0.650150851 |
| NCSTN | 0.649968604 |
| DNAH5 | 0.649683404 |
| PRRG4 | 0.649651425 |
| KLHL32 | 0.649634844 |
| VLDLR | 0.649040582 |
| ALS2CR12 | 0.648823488 |
| RASIP1 | 0.6479115 |
| RFXANK | 0.647041118 |
| PTP4A1 | 0.646627159 |
| C12orf80 | 0.646436538 |
| SH3BGR | 0.646350112 |
| PSCA | 0.645604253 |
| GSDMC | 0.645368073 |
| PPARG | 0.645147812 |
| EYA1 | 0.644916953 |
| USP32 | 0.644773585 |
| PAQR9 | 0.644688806 |
| CNGA1 | 0.644643397 |
| RHOH | 0.644539794 |
| HDAC4 | 0.644319168 |
| KRT2 | 0.643950598 |
| RAB23 | 0.643735074 |
| ARID5B | 0.643002553 |
| VRTN | 0.641727124 |
| IL11RA | 0.641383309 |
| ABI3BP | 0.640363631 |
| PZP | 0.640191307 |
| IBSP | 0.64004856 |
| RSPO1 | 0.639490538 |
| TMEM67 | 0.639203979 |
| USP15 | 0.638990803 |
| ALDHIL1 | 0.638913453 |
| C16orf72 | 0.638765706 |
| TMED4 | 0.638603716 |
| TRIM46 | 0.638400607 |
| IL33 | 0.638374614 |
| TDRD1 | 0.635966215 |
| LURAP1L | 0.635951398 |
| ATP13A5 | 0.635797172 |
| STOML1 | 0.635691953 |
| ADRA1A | 0.63523739 |
| DNAH14 | 0.635194535 |
| TF | 0.634799737 |
| HMGA1 | 0.634667901 |
| PRSS8 | 0.633657643 |
| LOC284898 | 0.633608928 |
| PIP5K1B | 0.633569385 |
| SVOPL | 0.632865818 |
| BPIFB1 | 0.632633977 |
| DEFA6 | 0.632094372 |
| GOLIM4 | 0.631263665 |
| SH2D7 | 0.630790181 |
| DONSON | 0.630553746 |
| MALL | 0.630513792 |
| LIN54 | 0.630027304 |
| C7orf34 | 0.629306655 |
| XDH | 0.628630112 |
| FAM60A | 0.628143258 |
| SLC13A4 | 0.626826121 |
| GFI1B | 0.626803213 |
| UQCRFS1 | 0.626613024 |
| CEBPB | 0.626444386 |
| NUP210 | 0.626241198 |
| N4BP3 | 0.625661992 |
| TSSK1B | 0.62561564 |
| LBH | 0.625568432 |
| CORO7-PAM16 | 0.625032681 |
| MPPED2 | 0.624950865 |
| INTS1 | 0.624767989 |
| CHRNA1 | 0.624267955 |
| SH2D4A | 0.624008301 |
| FBXW7 | 0.623654537 |
| OXCT1 | 0.623220407 |
| HSD17B14 | 0.623175672 |
| MTMR4 | 0.623030969 |
| PDLIM3 | 0.622695768 |
| CD302 | 0.622406992 |
| C5orf63 | 0.622330614 |
| GALNT2 | 0.621595312 |
| DAAM2 | 0.621318088 |
| IGLL1 | 0.621202916 |
| COBL | 0.620905213 |
| CLC | 0.619989207 |
| KHDC1L | 0.619883103 |
| CXCR6 | 0.619872726 |
| NOL4 | 0.618377165 |
| CHL1 | 0.618179007 |
| SERPINB6 | 0.617875793 |
| S100P | 0.617858063 |
| TRPV6 | 0.617714151 |
| ZNF687 | 0.617419761 |
| BLVRA | 0.616605942 |
| ESRP1 | 0.616400849 |
| C9orf152 | 0.616397355 |
| OR9Q2 | 0.616155798 |
| RP1L1 | 0.615993992 |
| C10orf82 | 0.615835592 |
| CYP4F11 | 0.61562244 |
| OR52N4 | 0.615516583 |
| EPGN | 0.614659067 |
| CCR9 | 0.614300185 |
| RRP15 | 0.614188728 |
| KLHDC7A | 0.614155292 |
| PHTF2 | 0.6139468 |
| HIVEP3 | 0.613752504 |
| OR52N5 | 0.613554943 |
| RGMB | 0.613251704 |
| NSL1 | 0.612958326 |
| MOBP | 0.612716852 |
| SLC10A6 | 0.612611992 |
| KIF5C | 0.612329221 |
| C4orf45 | 0.612200264 |
| FAM107B | 0.612077065 |
| DGKA | 0.611522244 |
| SH2D3A | 0.611187188 |
| PCK1 | 0.611029596 |
| SLC5A4 | 0.610940789 |
| TECTA | 0.61067127 |
| DENND6A | 0.610624488 |
| OR13C8 | 0.610124656 |
| PPDPF | 0.609532251 |
| NCAM1 | 0.609489418 |
| KCNG1 | 0.609001824 |
| LOC100132813 | 0.608927856 |
| PGC | 0.608468469 |
| HMGCLL1 | 0.608196278 |
| ZBTB10 | 0.608122017 |
| AGAP1 | 0.607175249 |
| GIMD1 | 0.607120102 |
| SLC30A1 | 0.604403935 |
| AGXT | 0.604389395 |
| NODAL | 0.603904802 |
| NME1-NME2 | 0.603808378 |
| PTPRZ1 | 0.603695345 |
| DENR | 0.603688408 |
| MS4A8 | 0.603562587 |
| C14orf132 | 0.60322968 |
| SOX6 | 0.603139709 |
| C17orf99 | 0.602945197 |
| UGT1A1 | 0.602445556 |
| PDILT | 0.602195576 |
| NOL11 | 0.601659354 |
| VSX2 | 0.601160197 |
| LOC105372977 | 0.60111929 |
| PIEZO2 | 0.601117662 |
| IL20RB | 0.601093712 |
| CACNA1E | 0.600903176 |
| CHRNA7 | 0.600697629 |
| HNRNPA2B1 | 0.600636199 |
| KRTAP3-1 | 0.599771794 |
| MCM2 | 0.59912486 |
| SPINT4 | 0.598323219 |
| FBXL7 | 0.597783597 |
| HYDIN | 0.597276339 |
| DEFB4B | 0.597211316 |
| TMEM213 | 0.597149005 |
| FHDC1 | 0.596899557 |
| SNAI2 | 0.596822353 |
| MTUS1 | 0.596323142 |
| ANKK1 | 0.596167616 |
| GNPTAB | 0.596124267 |
| CDCP2 | 0.596011639 |
| FKBPL | 0.595822297 |
| SULT6B1 | 0.595783361 |
| BARX1 | 0.595706643 |
| USP17L8 | 0.595704453 |
| PCDHB2 | 0.595577511 |
| TMEM237 | 0.595539729 |
| GTSF1L | 0.594573455 |
| TSPAN8 | 0.593929609 |
| KALRN | 0.59362093 |
| CHRNA9 | 0.592781487 |
| FUT8 | 0.592032962 |
| OTOR | 0.59114938 |
| PEX5L | 0.590573034 |
| C16orf45 | 0.58975089 |
| SPATA19 | 0.589460197 |
| ITGB6 | 0.589278431 |
| ANKRD46 | 0.588422837 |
| CFHR5 | 0.588358138 |
| PNMAL1 | 0.588142891 |
| MANSC1 | 0.588099301 |
| PCDHB4 | 0.587499315 |
| BBOX1 | 0.587489944 |
| GLDN | 0.587401442 |
| BAZ1A | 0.586790009 |
| TACC2 | 0.58665477 |
| TANC2 | 0.586366335 |
| RDH13 | 0.58628501 |
| C18orf8 | 0.586050779 |
| PHLDB3 | 0.585744219 |
| ARNT | 0.58548736 |
| GPR75-ASB3 | 0.584943858 |
| SGK223 | 0.584720751 |
| PI4K2A | 0.584710637 |
| KIAA1217 | 0.584169816 |
| LAPTM4B | 0.583756041 |
| LOC645177 | 0.583702292 |
| RBKS | 0.582210743 |
| GRM3 | 0.581975824 |
| BSPRY | 0.581938975 |
| TMX3 | 0.581411669 |
| WLS | 0.581323027 |
| MYOT | 0.581261407 |
| PYROXD1 | 0.580857069 |
| TNPO3 | 0.580847445 |
| MCF2L | 0.580664533 |
| CNIH3 | 0.580644385 |
| ZNF138 | 0.580516134 |
| TNP1 | 0.580508156 |
| SPPL2C | 0.580115072 |
| GOLGA6L9 | 0.579940888 |
| HINT1 | 0.579696265 |
| HIST1H4F | 0.579691165 |
| SASH1 | 0.579608327 |
| ZNF233 | 0.579459896 |
| GDF10 | 0.578909435 |
| LRRIQ3 | 0.578503128 |
| DTNA | 0.578298651 |
| PPP2R3A | 0.57823638 |
| IVL | 0.578195473 |
| STRA6 | 0.577968683 |
| JAZF1 | 0.577699784 |
| IL6 | 0.577605002 |
| PDZRN3 | 0.577523591 |
| MYCL | 0.577338945 |
| COMT | 0.577058192 |
| DAD1 | 0.57662311 |
| ZNF408 | 0.576568339 |
| CDH7 | 0.576462982 |
| GJA1 | 0.575985889 |
| HEY2 | 0.575581782 |
| C11orf44 | 0.575435172 |
| KNG1 | 0.575250497 |
| SLC22A1 | 0.574946558 |
| USP17L18 | 0.573743934 |
| CXXC4 | 0.573727414 |
| HMGN3 | 0.57338145 |
| KANSL1L | 0.573235345 |
| TK2 | 0.572940029 |
| AGR2 | 0.572385046 |
| KLHL31 | 0.572365547 |
| RRM2 | 0.571977539 |
| CPZ | 0.571710848 |
| FNDC8 | 0.571507032 |
| MTRNR2L9 | 0.571090358 |
| OR4F6 | 0.570410379 |
| LOC388780 | 0.570183265 |
| RASD2 | 0.569831116 |
| TEX10 | 0.569706617 |
| RHPN1 | 0.569690366 |
| SLC6A18 | 0.569446586 |
| TNS4 | 0.569440121 |
| XYLT2 | 0.568545335 |
| PSMF1 | 0.568304985 |
| HMGB4 | 0.567335994 |
| RBMS1 | 0.567062076 |
| PCDH18 | 0.567024069 |
| FAM84A | 0.566483535 |
| SPX | 0.566358142 |
| C12orf74 | 0.566306716 |
| BRD4 | 0.566281438 |
| TAT | 0.566004312 |
| SNRPD3 | 0.565792131 |
| LOC100130880 | 0.565446489 |
| CLDN12 | 0.565445694 |
| CA1 | 0.565391704 |
| TPH1 | 0.564649586 |
| SERPINI1 | 0.564197447 |
| CCDC125 | 0.56414466 |
| CD3D | 0.56403072 |
| CD2BP2 | 0.563847743 |
| DPY19L4 | 0.56308503 |
| CASR | 0.5623909 |
| CA8 | 0.562366737 |
| NRN1L | 0.561486121 |
| RBP2 | 0.56067759 |
| SLX4IP | 0.560605274 |
| C9orf16 | 0.560572463 |
| KCNA5 | 0.559826965 |
| SPATA18 | 0.55928074 |
| IMMP2L | 0.559212741 |
| DSC1 | 0.558984466 |
| C5orf49 | 0.558964539 |
| MYPN | 0.558956037 |
| ADNP2 | 0.558924042 |
| MSANTD3 | 0.558877725 |
| NOTCH2NL | 0.558761848 |
| PPIC | 0.558616524 |
| MMAA | 0.558444529 |
| CMYA5 | 0.558334292 |
| SNAP25 | 0.55789178 |
| USP25 | 0.557860574 |
| NMBR | 0.55755988 |
| ADIPOQ | 0.5572082 |
| MBIP | 0.556807615 |
| RRP1 | 0.555648682 |
| MMD | 0.554956405 |
| GCNT1 | 0.554907339 |
| TPBG | 0.554888948 |
| HOXC10 | 0.554714885 |
| STC1 | 0.554624855 |
| RAD21 | 0.554513363 |
| CTSZ | 0.554434452 |
| IL23R | 0.554171481 |
| SOX9 | 0.554111065 |
| CD164L2 | 0.553794451 |
| UGT2A1 | 0.553162946 |
| RNF152 | 0.552463065 |
| AQP4 | 0.552282885 |
| OR8A1 | 0.551953392 |
| TMEM91 | 0.551835593 |
| FBN3 | 0.551474621 |
| MPZL2 | 0.551467936 |
| CAMK1G | 0.551464377 |
| DAGLB | 0.551413659 |
| STK38 | 0.550719346 |
| SLC5A7 | 0.550445987 |
| WDR25 | 0.549949739 |
| NSG1 | 0.549810235 |
| SLC20A2 | 0.549308094 |
| TEX26 | 0.549161089 |
| TRIM35 | 0.548760137 |
| CHRNA3 | 0.54873554 |
| CCDC30 | 0.548404593 |
| RFPL4B | 0.548200876 |
| NOL10 | 0.547993385 |
| SETDB1 | 0.547279969 |
| OLFM3 | 0.547278505 |
| KIAA1549L | 0.547257489 |
| PRDM4 | 0.547239507 |
| ASB15 | 0.546964284 |
| C11orf86 | 0.546719109 |
| RCAN3 | 0.54647922 |
| PLPPR4 | 0.546438168 |
| NREP | 0.546406346 |
| LAD1 | 0.546384545 |
| ABCC2 | 0.546356578 |
| SLC6A11 | 0.545840915 |
| TRAPPC2L | 0.545739739 |
| SLC25A24 | 0.544655682 |
| GGACT | 0.54429028 |
| PCDHGB1 | 0.544059497 |
| PATE2 | 0.543764121 |
| COX6C | 0.543730708 |
| ARHGDIB | 0.543670724 |
| PIP | 0.543263808 |
| MGP | 0.542637256 |
| BRE | 0.542550168 |
| TMEM30B | 0.542382399 |
| CRABP1 | 0.541981056 |
| MYOF | 0.541623942 |
| WRB-SH3BGR | 0.541398628 |
| SCAND1 | 0.541378765 |
| PCDHGA6 | 0.541302811 |
| KRTAP9-2 | 0.541161961 |
| APOL5 | 0.540910039 |
| FMN1 | 0.540693808 |
| DEFB1 | 0.540471017 |
| DUSP28 | 0.540387402 |
| YWHAQ | 0.540250395 |
| DENND1B | 0.540105311 |
| USP54 | 0.538983655 |
| CTXN3 | 0.538627054 |
| PCDHB16 | 0.538581173 |
| FN1 | 0.538497337 |
| ANGPT2 | 0.538222899 |
| ERICH2 | 0.537974444 |
| RASSF9 | 0.537881716 |
| LY6K | 0.53750734 |
| SAMD10 | 0.537154525 |
| WASF3 | 0.537062791 |
| ACTR1B | 0.536564748 |
| TOMM34 | 0.53627922 |
| TNFAIP8L1 | 0.535982712 |
| SBF1 | 0.535324612 |
| TBC1D31 | 0.535313855 |
| IDH1 | 0.535079415 |
| ATP2B2 | 0.535060785 |
| ASPG | 0.534622283 |
| RNF150 | 0.534486681 |
| FCN2 | 0.534466795 |
| LTF | 0.534422905 |
| PROK1 | 0.534243892 |
| SDF4 | 0.534014228 |
| PIWIL3 | 0.533612216 |
| PCBP3 | 0.533531625 |
| FRMPD2 | 0.533447676 |
| C2CD4B | 0.533177199 |
| AKR1C3 | 0.532977777 |
| GART | 0.532364635 |
| SEMA4G | 0.532245327 |
| VAMP4 | 0.532126797 |
| SKIDA1 | 0.532121774 |
| MPP4 | 0.531695819 |
| PKD1L1 | 0.531513479 |
| CLEC3B | 0.531294856 |
| C21orf91 | 0.531058778 |
| KRTAP9-9 | 0.530983932 |
| GPR6 | 0.53076393 |
| HSPB2 | 0.530572714 |
| MC2R | 0.530283171 |
| MUC5B | 0.530126247 |
| HTR5A | 0.530106609 |
| PPTC7 | 0.530057127 |
| RPS19BP1 | 0.529707857 |
| FUT3 | 0.529451882 |
| MELK | 0.529434377 |
| RTFDC1 | 0.529367517 |
| PLAC8 | 0.529064209 |
| DEFB106B | 0.529014347 |
| HRH1 | 0.528809477 |
| IFT20 | 0.528725265 |
| DNASE1 | 0.528498182 |
| PREPL | 0.528080083 |
| SLC39A10 | 0.52770944 |
| PGBD2 | 0.527599861 |
| SEMA6A | 0.52756242 |
| CDC42BPG | 0.527496854 |
| TECTB | 0.527330856 |
| KAZN | 0.527150815 |
| TNFAIP1 | 0.527061832 |
| KDM5B | 0.527007294 |
| IL17C | 0.526548791 |
| MYOZ2 | 0.526523979 |
| ILDR1 | 0.526366254 |
| OAZ1 | 0.526073731 |
| BCL6B | 0.525607872 |
| WARS | 0.525429068 |
| ALDH8A1 | 0.525311906 |
| UTS2B | 0.525075003 |
| MYF5 | 0.524961211 |
| RASGRP3 | 0.524638496 |
| PDLIM4 | 0.524605951 |
| CCDC67 | 0.524585423 |
| IQCB1 | 0.524528756 |
| CAPN9 | 0.524372018 |
| ARHGAP8 | 0.524326598 |
| TAS2R30 | 0.524115714 |
| FAM24A | 0.524045456 |
| CDCP1 | 0.523731332 |
| SNRNP27 | 0.523637547 |
| FAM205A | 0.523318763 |
| CNTN3 | 0.522824349 |
| SELM | 0.522812778 |
| SMG8 | 0.52242517 |
| LONP1 | 0.522341979 |
| TSKU | 0.522279948 |
| CDH1 | 0.522065026 |
| ADCYAP1R1 | 0.521747089 |
| PIGP | 0.52127998 |
| KIF6 | 0.520988065 |
| SORCS1 | 0.520943026 |
| ZBTB43 | 0.520676007 |
| GRIN3A | 0.520512043 |
| OR4S1 | 0.520359549 |
| KRT80 | 0.520288457 |
| LRRC37A2 | 0.519907663 |
| FOXP1 | 0.519725257 |
| PKIG | 0.519538013 |
| MVB12A | 0.51911703 |
| PRPF40B | 0.519046756 |
| GTPBP2 | 0.518783844 |
| FCHSD2 | 0.518505781 |
| SUSD2 | 0.51850053 |
| UCN3 | 0.518268871 |
| FAM19A3 | 0.518185524 |
| FMN2 | 0.518016396 |
| ADH5 | 0.517720667 |
| WIPF3 | 0.517669753 |
| XRN1 | 0.517646797 |
| ANKRD61 | 0.517157913 |
| FBXO38 | 0.517139416 |
| PDHA2 | 0.517086181 |
| C9orf3 | 0.516687943 |
| BAZ2B | 0.516663111 |
| MT4 | 0.516217099 |
| TNC | 0.516031649 |
| COQ3 | 0.515953853 |
| NPIPB6 | 0.515948267 |
| NDUFV1 | 0.515718488 |
| ABCC12 | 0.51563297 |
| LACTBL1 | 0.515372576 |
| SGCG | 0.51535898 |
| DDR1 | 0.514856213 |
| ASPN | 0.514848651 |
| HPX | 0.514686856 |
| MMP21 | 0.51455296 |
| PNLIPRP3 | 0.514405658 |
| RPL9 | 0.513855324 |
| H2AFY2 | 0.513462894 |
| HNMT | 0.512841333 |
| ACSM1 | 0.512473199 |
| OXA1L | 0.512456651 |
| MPRIP | 0.512279852 |
| PKD1L2 | 0.511850222 |
| CCDC185 | 0.511446803 |
| TMOD1 | 0.511339797 |
| KRT20 | 0.511078944 |
| PCP4 | 0.510779224 |
| C1orf43 | 0.510739791 |
| TIGD2 | 0.510679028 |
| ACSM2B | 0.510670492 |
| ADGRF4 | 0.509812607 |
| PCDHGB2 | 0.509336498 |
| C20orf202 | 0.509068115 |
| PTGES | 0.508886985 |
| TAS1R2 | 0.508732833 |
| RPH3AL | 0.508681962 |
| ZMYND8 | 0.50828232 |
| EIF4EBP1 | 0.508185471 |
| CCP110 | 0.508012779 |
| ABCC9 | 0.507664219 |
| ADNP | 0.507646534 |
| RADIL | 0.507587283 |
| GRIA1 | 0.507461552 |
| USP5 | 0.507418542 |
| STX3 | 0.507173222 |
| STMND1 | 0.506931992 |
| ZFAND5 | 0.506878276 |
| RNF112 | 0.506815035 |
| MRGPRX4 | 0.506779306 |
| BTNL8 | 0.50677315 |
| HLA-DQB2 | 0.506555067 |
| CREBBP | 0.506334346 |
| FZD5 | 0.505969931 |
| HINFP | 0.505835048 |
| SEMA6D | 0.505711063 |
| UMODL1 | 0.505631722 |
| CARD19 | 0.505486414 |
| TTC39C | 0.504767027 |
| MTMR11 | 0.504659773 |
| SULT2B1 | 0.504656231 |
| INSL4 | 0.504548736 |
| PACRG | 0.504440025 |
| MYSM1 | 0.503973254 |
| SERPINF2 | 0.503961841 |
| EMCN | 0.503826367 |
| PLPP4 | 0.503685295 |
| THNSL2 | 0.503515189 |
| KCNA6 | 0.503333096 |
| CUZD1 | 0.503318175 |
| DENND1A | 0.502954307 |
| FRG2 | 0.502941411 |
| ZNF532 | 0.502803667 |
| PIP4K2C | 0.502751527 |
| TRABD2B | 0.502731209 |
| SULT1E1 | 0.502286675 |
| PLCB1 | 0.502275706 |
| GNRH1 | 0.502194321 |
| ST6GALNAC5 | 0.502001446 |
| AURKA | 0.50181214 |
| TSHZ2 | 0.501481035 |
| TTLL4 | 0.501203394 |
| HMGXB3 | 0.50081488 |
| PMPCA | 0.5007765 |
| SEPT1 | 0.500496928 |
| ACOT7 | 0.500437214 |
| REM2 | 0.500113491 |
| FIBIN | 0.500075956 |
| MTA3 | 0.500010728 |

TABLE 4
Gene Panel: Cancer Panel 2

| Gene Name | Hedges' g |
|---|---|
| KIR3DL2 | 1.238941321 |
| LHX4 | 1.18910963 |
| STX10 | 1.162294752 |
| USP17L19 | 1.125373645 |
| KIF22 | 1.11240923 |
| PCDHGA6 | 1.086802175 |
| BZW1 | 1.06417008 |
| AKT1S1 | 1.058565154 |
| TMCC3 | 1.048079667 |
| VIM | 1.047039919 |
| SIGIRR | 1.044319986 |
| HS6ST1 | 1.027778533 |
| ANKMY2 | 1.021636059 |
| DRD3 | 1.018893256 |
| RNASEH1 | 1.013416711 |
| PDE3B | 1.011217022 |
| IZUMO4 | 1.002567804 |
| CD24 | 1.001992292 |
| KNSTRN | 0.995692394 |
| FAM45A | 0.993271308 |
| PRAMEF15 | 0.988116782 |
| RAD52 | 0.985572105 |
| GATSL2 | 0.984813866 |
| DNTT | 0.984683649 |
| RNF141 | 0.982187151 |
| ZNRD1 | 0.98086868 |
| MTRF1 | 0.979080167 |
| NPIPB15 | 0.971262343 |
| PREX2 | 0.970911669 |
| CLPB | 0.966545248 |
| CLCN7 | 0.966236751 |
| CNPPD1 | 0.963119531 |
| DEFB113 | 0.96138802 |
| HYAL2 | 0.957933008 |
| LYST | 0.94738202 |
| ERP29 | 0.946415111 |
| CDH3 | 0.942560796 |
| TRERF1 | 0.938666596 |
| UBE2K | 0.938146324 |
| UBTFL1 | 0.933615964 |
| HSPA1B | 0.92501753 |
| OR5T3 | 0.923530086 |
| SLC26A7 | 0.922139287 |
| DCDC5 | 0.921995714 |
| DOC2B | 0.916856229 |
| NFIL3 | 0.912892119 |
| GALE | 0.910961021 |
| HNRNPCL2 | 0.908015983 |
| STX3 | 0.906189142 |
| UHRF1BP1L | 0.90611206 |
| KIR2DL3 | 0.905820948 |
| HYAL1 | 0.904426116 |
| ADAMTSL4 | 0.904340099 |
| MAP2K3 | 0.89436535 |
| ARL17B | 0.89247995 |
| TRAP1 | 0.888443177 |
| FBXO27 | 0.887869085 |
| UBN1 | 0.884628772 |
| RPS7 | 0.88394053 |
| POLR3B | 0.882162413 |
| ABHD17A | 0.87991729 |
| RIMKLB | 0.87770779 |
| EPS15L1 | 0.876753512 |
| HM13 | 0.875474947 |
| MXD4 | 0.872305673 |
| EID2B | 0.870126212 |
| MERTK | 0.867289176 |
| ADAM21 | 0.864641519 |
| APMAP | 0.864553169 |
| ACLY | 0.862485754 |
| LARP7 | 0.861293895 |
| SLCO1A2 | 0.861127946 |
| MDK | 0.859588849 |
| H3F3C | 0.859583259 |
| PPM1A | 0.857109956 |
| SIPA1L1 | 0.852972691 |
| HOXA7 | 0.85111905 |
| PRAMEF6 | 0.850856041 |
| TPP2 | 0.849772274 |
| CYP27A1 | 0.849702784 |
| MOSPD3 | 0.845784043 |
| TUSC1 | 0.845767512 |
| PSMC6 | 0.844681367 |
| ATP6V1G2 | 0.843633227 |
| COQ9 | 0.841932054 |
| LIPT2 | 0.840697337 |
| ARPC5 | 0.84045044 |
| EPN2 | 0.839896732 |
| PRNP | 0.839559073 |
| MTUS1 | 0.838380382 |
| MPP6 | 0.83736276 |
| DDX27 | 0.83629692 |
| TBCC | 0.836062658 |
| BAP1 | 0.833567668 |
| CSGALNACT1 | 0.831724877 |
| NPTN | 0.828224461 |
| ADAM15 | 0.826537151 |
| IRF2 | 0.825562988 |
| PPP1R3B | 0.824865998 |
| MARS2 | 0.822983788 |
| TAP1 | 0.822479948 |
| RBM4 | 0.821896917 |
| TMEM242 | 0.821696762 |
| FAM126A | 0.821526303 |
| PYGB | 0.820284445 |
| WNT4 | 0.820260729 |
| SLC52A1 | 0.817140115 |
| DEFB105B | 0.816495408 |
| MXRA7 | 0.815596059 |
| EBF1 | 0.815254027 |
| HCK | 0.813303534 |
| C16orf91 | 0.812660463 |
| LHX1 | 0.812551645 |
| LYG1 | 0.812078252 |
| CD2BP2 | 0.811824094 |
| DENND4B | 0.811272665 |
| POLN | 0.810769855 |
| EIF3A | 0.809185 |
| PCTP | 0.80715311 |
| XRCC6 | 0.80713437 |
| PTP4A1 | 0.807118992 |
| WDR77 | 0.806137702 |
| FGFR1OP | 0.805784365 |
| ARHGAP26 | 0.805463905 |
| ANG | 0.804599735 |
| LMTK3 | 0.803444778 |
| TMEM101 | 0.802521209 |
| PCDHB13 | 0.799299357 |
| ADGRF3 | 0.798079552 |
| ARL16 | 0.79517746 |
| BTC | 0.795097751 |
| OR56A4 | 0.794890718 |
| IREB2 | 0.793705211 |
| TP53I3 | 0.792870061 |
| EXOSC10 | 0.792749238 |
| GIPC2 | 0.79127223 |
| FSCN1 | 0.790586535 |
| RAB40C | 0.789884993 |
| ASXL1 | 0.78843697 |
| IRX5 | 0.788312054 |
| OR2A42 | 0.788311498 |
| DGCR14 | 0.788220296 |
| PLEKHA5 | 0.787548136 |
| IL18R1 | 0.787076888 |
| TNFRSF1A | 0.786505872 |
| OTOGL | 0.78543314 |
| DGCR6L | 0.785421864 |
| EIF3C | 0.785009858 |
| ABI1 | 0.78437593 |
| CORO7-PAM16 | 0.784134368 |
| DEPDC1B | 0.78281904 |
| C6orf1 | 0.781622406 |
| PRKRA | 0.780094105 |
| C16orf13 | 0.777695163 |
| NAT6 | 0.777668405 |
| NOA1 | 0.777037988 |
| CPT1C | 0.776804245 |
| TRMT12 | 0.775579575 |
| VIT | 0.774122991 |
| SAMD1 | 0.77405407 |
| RPL28 | 0.773096217 |
| H2AFJ | 0.772984527 |
| NME3 | 0.771399365 |
| RAB36 | 0.771290945 |
| RNASEH2C | 0.769682804 |
| ZC3H3 | 0.769002934 |
| CHMP4B | 0.768902742 |
| PNPLA6 | 0.767833386 |
| WIPI2 | 0.767256496 |
| GABRR1 | 0.767132804 |
| BMP5 | 0.765571504 |
| ACSM2A | 0.764190027 |
| GTF3C5 | 0.763780606 |
| CYP26B1 | 0.763584663 |
| STAM | 0.763584252 |
| IGFBP2 | 0.763513968 |
| RNF32 | 0.762470335 |
| DTNBP1 | 0.762452466 |
| LRPAP1 | 0.762143319 |
| OR2T35 | 0.761519254 |
| GOS2 | 0.760233467 |
| NUTM2G | 0.759966939 |
| RFX2 | 0.7597331 |
| SYNC | 0.759186052 |
| HSF4 | 0.757938603 |
| ATOH8 | 0.756268097 |
| ZNF839 | 0.75581403 |
| TAF1L | 0.755757212 |
| RPP38 | 0.755307925 |
| ANGPTL4 | 0.75499979 |
| SBF1 | 0.754620334 |
| TSSC4 | 0.754148816 |
| PAFAH2 | 0.753546459 |
| ERAL1 | 0.753112237 |
| CSRP1 | 0.752637437 |
| TRIO | 0.751248857 |
| CACNG8 | 0.750307781 |
| TMED5 | 0.749240936 |
| RRP9 | 0.748509811 |
| ATRIP | 0.748473851 |
| BIRC5 | 0.748233055 |
| RASSF8 | 0.747448113 |
| TM4SF18 | 0.744832583 |
| RGS17 | 0.7446511 |
| UBE2QL1 | 0.744501936 |
| DNAJC11 | 0.744070496 |
| TRNP1 | 0.742676033 |
| PPP4R3B | 0.742671538 |
| DGAT1 | 0.74263777 |
| SOX6 | 0.742429874 |
| PTHLH | 0.742202299 |
| TAAR5 | 0.74147462 |
| MMP10 | 0.741065408 |
| FAM217B | 0.741034674 |
| CBARP | 0.740438847 |
| USP17L8 | 0.740136583 |
| MRPL20 | 0.738576507 |
| THSD4 | 0.737499433 |
| DSTYK | 0.737342222 |
| PDLIM4 | 0.737032836 |
| ARL2 | 0.736377049 |
| TMEM191C | 0.733616168 |
| VSX1 | 0.732981966 |
| GHR | 0.732970451 |
| RPL34 | 0.732889015 |
| PLOD3 | 0.731200389 |
| C17orf99 | 0.73111384 |
| CRYAA | 0.730892461 |
| RPS8 | 0.730660025 |
| JMJD4 | 0.729837395 |
| FAM134B | 0.729770651 |
| NDUFC2-KCTD14 | 0.729532761 |
| ZFAND3 | 0.729500674 |
| CHMP5 | 0.72885287 |
| PCDHGA4 | 0.728807972 |
| SLC2A4 | 0.727877056 |
| ELF2 | 0.726951104 |
| GPCPD1 | 0.724341317 |
| NBPF6 | 0.724040023 |
| DNAH17 | 0.723158308 |
| EFNB2 | 0.723036398 |
| FDFT1 | 0.72290095 |
| HMCES | 0.720632213 |
| IGSF9B | 0.720296139 |
| SF3A1 | 0.719697107 |
| PIGZ | 0.719622517 |
| SPPL2B | 0.718416325 |
| GABARAP | 0.718200171 |
| FAM107A | 0.71804854 |
| CLTCL1 | 0.717977083 |
| EMC6 | 0.717833942 |
| RTKN | 0.717170397 |
| PPP2R5E | 0.71704722 |
| FDPS | 0.71693487 |
| CARD18 | 0.716743662 |
| RNF44 | 0.71668867 |
| ZNF425 | 0.71662591 |
| GIMAP4 | 0.71640603 |
| C1QTNF6 | 0.715944296 |
| PARP3 | 0.715491347 |
| RPIA | 0.715359444 |
| TRPM6 | 0.714267921 |
| ATXN2L | 0.713346501 |
| TFDP2 | 0.712980211 |
| S100A3 | 0.711127005 |
| PEX11B | 0.70979849 |
| ACTN4 | 0.709440611 |
| EIF4A1 | 0.708107318 |
| PROCR | 0.707667328 |
| ISCA2 | 0.706772151 |
| EIF2S1 | 0.706305681 |
| DCAF4L1 | 0.705669603 |
| OR5T2 | 0.70498758 |
| BYSL | 0.704950142 |
| PXMP4 | 0.704066537 |
| SF1 | 0.703691642 |
| PRAMEF13 | 0.703485077 |
| SULT6B1 | 0.703142685 |
| MCM4 | 0.702350859 |
| STRA13 | 0.702163283 |
| MYL6 | 0.702111431 |
| CANX | 0.701306018 |
| GK5 | 0.701247498 |
| FAM155A | 0.700352221 |
| SLC10A2 | 0.699991952 |
| RBMS1 | 0.699386666 |
| FOXK2 | 0.699332824 |
| SPATA31A7 | 0.698090723 |
| GULP1 | 0.697845954 |
| MAP3K6 | 0.697774593 |
| ARAP3 | 0.697702345 |
| TROVE2 | 0.697319794 |
| BCL2L14 | 0.697138795 |
| PALMD | 0.697102684 |
| HSP90B1 | 0.697051091 |
| HSF1 | 0.696672598 |
| ACAT2 | 0.696452963 |
| EFEMP2 | 0.696101297 |
| ZNF692 | 0.69515615 |
| NAA35 | 0.694835907 |
| MAP2K7 | 0.694729359 |
| TACSTD2 | 0.69353986 |
| RFXAP | 0.693503558 |
| CPB1 | 0.693442594 |
| CARS | 0.693137101 |
| TET1 | 0.692870904 |
| RABAC1 | 0.69184467 |
| MRM3 | 0.691310373 |
| ABHD14B | 0.691289775 |
| GSTA1 | 0.690325054 |
| ARFIP2 | 0.690321806 |
| DPY30 | 0.690092243 |
| C3orf14 | 0.688376924 |
| CARHSP1 | 0.688088584 |
| THAP3 | 0.687964039 |
| LOC100133267 | 0.68772163 |
| TAPBPL | 0.68696435 |
| SASH1 | 0.686565194 |
| ME3 | 0.686103774 |
| DHRS1 | 0.685939289 |
| SLC30A9 | 0.685610374 |
| SLC9A3R1 | 0.685469188 |
| KIF5C | 0.6854225 |
| STX4 | 0.68491431 |
| B3GNT8 | 0.684554857 |
| FUOM | 0.684505738 |
| PREB | 0.684288951 |
| TTC9 | 0.683333622 |
| WDR64 | 0.683233599 |
| CLDND2 | 0.682799696 |
| DNAJA3 | 0.682643885 |
| DPH3 | 0.682223437 |
| IAH1 | 0.681650951 |
| RAB27A | 0.680441203 |
| SERINC5 | 0.680187278 |
| NBPF20 | 0.679085584 |
| GUK1 | 0.679013337 |
| C4orf45 | 0.678849166 |
| ELK3 | 0.678586388 |
| TNFRSF10A | 0.678447857 |
| TMEM87B | 0.677864992 |
| CDCA3 | 0.677785588 |
| PPP1R2 | 0.677686065 |
| SLC35E3 | 0.677585186 |
| CACNA2D1 | 0.677531604 |
| PRPF4B | 0.677345066 |
| LOC388780 | 0.677040657 |
| HEXIM2 | 0.67702485 |
| EHD1 | 0.676566413 |
| RAI14 | 0.676131849 |
| MPLKIP | 0.676032549 |
| SPATA31A1 | 0.676003063 |
| DAAM1 | 0.675830512 |
| PDXK | 0.675101604 |
| SPIRE2 | 0.674244231 |
| HINT3 | 0.674120262 |
| OSBPL5 | 0.673874158 |
| USP17L11 | 0.673727818 |
| OR52E6 | 0.673619318 |
| VCPIP1 | 0.673615321 |
| TPCN1 | 0.673496438 |
| ABL2 | 0.67338633 |
| CNN2 | 0.672390066 |
| ZMIZ2 | 0.671770882 |
| FAM175B | 0.671517638 |
| ARFGAP2 | 0.671137432 |
| EMSY | 0.67108512 |
| SPTBN1 | 0.670774371 |
| PRDM13 | 0.670241356 |
| ELAVL2 | 0.669859302 |
| NR2C2AP | 0.669758545 |
| ZNF331 | 0.669738073 |
| GLYATL3 | 0.668414247 |
| GRB2 | 0.667555438 |
| AK2 | 0.66752623 |
| VWA2 | 0.666756027 |
| CD248 | 0.666074289 |
| DENND1A | 0.66570893 |
| PLK3 | 0.665455589 |
| RRP8 | 0.6648838 |
| DEXI | 0.664692963 |
| RPL17-C18orf32 | 0.664692164 |
| EIF5A | 0.663701961 |
| PIK3R2 | 0.663296538 |
| SPRR2D | 0.663252404 |
| STX1A | 0.663059924 |
| ZNF326 | 0.662992167 |
| MTRNR2L1 | 0.662953586 |
| OR51A4 | 0.662739244 |
| C9orf3 | 0.662724861 |
| ADAMTS7 | 0.662680262 |
| ZNF623 | 0.662543698 |
| TRIM64B | 0.661409792 |
| PAG1 | 0.661379836 |
| MTURN | 0.661299355 |
| GSDMD | 0.66075378 |
| EMCN | 0.660622975 |
| GOLM1 | 0.660061465 |
| KIFC2 | 0.65991329 |
| CERS2 | 0.659344353 |
| KLHL42 | 0.659032627 |
| HRH1 | 0.658922975 |
| RGS19 | 0.658905104 |
| GFI1B | 0.658829493 |
| FKBPL | 0.658312428 |
| CNN3 | 0.65801449 |
| PTK2B | 0.657762812 |
| NPFFR2 | 0.657681452 |
| IFRD2 | 0.657673581 |
| AKAP8L | 0.65754672 |
| TMEM95 | 0.65702095 |
| EXOC3 | 0.656774761 |
| PSD | 0.656655769 |
| GSKIP | 0.656136554 |
| COPRS | 0.656123827 |
| TECTA | 0.655734553 |
| TMEM218 | 0.655644495 |
| OSTM1 | 0.655229006 |
| KLHDC3 | 0.655032984 |
| HAND1 | 0.654812552 |
| ANGPTL1 | 0.654372183 |
| C7orf62 | 0.653882273 |
| OR13C5 | 0.653806968 |
| WDR43 | 0.653649148 |
| CCNO | 0.653623646 |
| NUDT22 | 0.653285574 |
| EDEM2 | 0.653253323 |
| SLC22A23 | 0.653228884 |
| LOC100289561 | 0.653199808 |
| PHKG1 | 0.65296713 |
| CXCL16 | 0.652650882 |
| EEF1AKMT1 | 0.652590285 |
| PTPRJ | 0.652532031 |
| RNF185 | 0.6524914 |
| ATF6B | 0.651704858 |
| LRRC45 | 0.651663304 |
| SERTAD2 | 0.651553557 |
| RPAP2 | 0.650747289 |
| CLN8 | 0.650283591 |
| POTEI | 0.64997718 |
| TAS2R8 | 0.64992918 |
| TMEM70 | 0.649676641 |
| TRIP13 | 0.649343851 |
| NT5C1B-RDH14 | 0.649238267 |
| SNTG1 | 0.649145705 |
| TBC1D30 | 0.648908345 |
| NUDT16 | 0.648902755 |
| GSG2 | 0.648786936 |
| ZNF80 | 0.648375537 |
| AMPD2 | 0.648221932 |
| SEPT5 | 0.647275492 |
| CEP104 | 0.647212644 |
| CFB | 0.646015742 |
| TRAPPC3L | 0.645934342 |
| ATP5D | 0.645914871 |
| PBOV1 | 0.64568897 |
| GNA14 | 0.645331762 |
| PCNX3 | 0.644768508 |
| GRAP | 0.644679579 |
| ZC3H14 | 0.643879128 |
| AZIN2 | 0.643634864 |
| ECH1 | 0.643204026 |
| SOX13 | 0.642934143 |
| CNNM2 | 0.642461076 |
| HAVCR1 | 0.642332376 |
| CEACAM19 | 0.642313529 |
| VAX2 | 0.641374276 |
| PTGIS | 0.641149252 |
| HNF1B | 0.641096286 |
| FIGNL2 | 0.639772329 |
| CGNL1 | 0.639744478 |
| BIVM | 0.639583968 |
| HOXA1 | 0.639559504 |
| LRRC37A3 | 0.639080956 |
| KRTAP1-3 | 0.638991641 |
| OR5H2 | 0.63872619 |
| TMEM143 | 0.638520444 |
| NCALD | 0.637860764 |
| HSPA14 | 0.63745309 |
| PYURF | 0.637396847 |
| IGF2 | 0.637286667 |
| FRS2 | 0.636551213 |
| PQLC2 | 0.636156764 |
| KRTAP5-3 | 0.635929761 |
| FAM76A | 0.635868548 |
| ATG9A | 0.635523401 |
| EPHX3 | 0.635105712 |
| SACM1L | 0.634683333 |
| S1PR5 | 0.634533806 |
| ZAR1L | 0.634349869 |
| ATOX1 | 0.634280542 |
| CPPED1 | 0.634267627 |
| SDCCAG3 | 0.633613062 |
| VAMP8 | 0.633423115 |
| CHST15 | 0.632458973 |
| CMTM4 | 0.632104705 |
| NOL9 | 0.631582442 |
| TMEM189-UBE2V1 | 0.630935644 |
| DNAJC4 | 0.630669922 |
| ENDOV | 0.630445586 |
| OR13G1 | 0.630291412 |
| PLN | 0.630098464 |
| TYSND1 | 0.62991323 |
| C16orf74 | 0.629792048 |
| PLGLB2 | 0.629713456 |
| TMEM99 | 0.628732868 |
| OXA1L | 0.6285828 |
| BLCAP | 0.62836947 |
| EPYC | 0.627843302 |
| FAM118B | 0.627091218 |
| NOL3 | 0.627014515 |
| RASGRF1 | 0.62616649 |
| COIL | 0.625167625 |
| IL33 | 0.624458383 |
| ALX1 | 0.623478743 |
| HMGCR | 0.623117409 |
| WNT7B | 0.623032482 |
| IP6K1 | 0.623007602 |
| CCDC80 | 0.622736452 |
| KLHL10 | 0.621558273 |
| IVD | 0.621393738 |
| PRSS33 | 0.621318449 |
| CLDN24 | 0.621037175 |
| FGFR4 | 0.620997093 |
| TPD52L2 | 0.620768375 |
| HPCAL1 | 0.620408643 |
| TSC22D4 | 0.620090784 |
| C16orf70 | 0.620086173 |
| IMPG1 | 0.619944003 |
| TSPAN11 | 0.619528345 |
| AFAP1L2 | 0.619254482 |
| POLR3H | 0.618798791 |
| PCDHB4 | 0.618705028 |
| OR5AN1 | 0.618547375 |
| GOLGA8B | 0.618417768 |
| FGF19 | 0.618406344 |
| OR2A2 | 0.618347562 |
| SPARCL1 | 0.618129814 |
| FAM69B | 0.618120139 |
| GLIS2 | 0.617622211 |
| C1QTNF5 | 0.617458071 |
| P2RY2 | 0.617315069 |
| AHRR | 0.616564127 |
| REV1 | 0.616404472 |
| TLCD1 | 0.616146494 |
| RASGRP3 | 0.615873635 |
| TRIM39-RPP21 | 0.615827956 |
| ASB7 | 0.615476011 |
| PPIC | 0.615366293 |
| SLF2 | 0.615313548 |
| ZNF865 | 0.615056671 |
| FAM189B | 0.614818201 |
| CAPNS2 | 0.614516464 |
| TSKU | 0.614306996 |
| FBXO47 | 0.614174301 |
| BST2 | 0.614010959 |
| SOX17 | 0.613983803 |
| BCL2L11 | 0.613695359 |
| C16orf86 | 0.613663253 |
| TRABD2A | 0.613490403 |
| MTF1 | 0.613302454 |
| USP19 | 0.613287702 |
| SMARCD3 | 0.613276772 |
| KCNE1 | 0.613239033 |
| PPL | 0.612975505 |
| OR2C1 | 0.61280829 |
| AFAP1 | 0.612806519 |
| OR5B12 | 0.612793576 |
| COX7A2L | 0.61261065 |
| SAP30L | 0.612495719 |
| FN3KRP | 0.61222513 |
| HIC2 | 0.611854344 |
| TBX6 | 0.611174543 |
| FAM135A | 0.611108439 |
| AMY1A | 0.61075252 |
| RNF133 | 0.610241511 |
| TMEM247 | 0.60965056 |
| ZNF580 | 0.609613289 |
| OR1A2 | 0.609431 |
| IKBIP | 0.609283272 |
| LAYN | 0.60902598 |
| PLEKHG1 | 0.608683709 |
| POLE3 | 0.608421248 |
| TRAM2 | 0.60786401 |
| DCN | 0.607818053 |
| E2F8 | 0.607795229 |
| RNF208 | 0.607449812 |
| PHF19 | 0.607329617 |
| ADAMTS3 | 0.6071696 |
| PRAMEF25 | 0.607016933 |
| MFN2 | 0.606774845 |
| SCD | 0.606753896 |
| RHBDL2 | 0.606567663 |
| PLAC8L1 | 0.606443826 |
| MCCC2 | 0.605703981 |
| FAU | 0.605702557 |
| RPL26 | 0.605621949 |
| RNH1 | 0.605196014 |
| SHCBP1L | 0.604676588 |
| FOXO1 | 0.604385359 |
| TIMM10 | 0.604134842 |
| AATF | 0.603527297 |
| CIPC | 0.603262695 |
| SSH3 | 0.603066397 |
| OR6Q1 | 0.602319405 |
| SWI5 | 0.602249902 |
| PDE1C | 0.601660294 |
| TPTE2 | 0.601557224 |
| IDO2 | 0.600979173 |
| NUAK1 | 0.600870187 |
| ARHGAP30 | 0.600706472 |
| TAS2R31 | 0.600450566 |
| PI4K2A | 0.599727895 |
| PTGES | 0.599634858 |
| LTBP4 | 0.598693801 |
| SYCP2 | 0.598194294 |
| COL5A2 | 0.598167737 |
| OXNAD1 | 0.597975113 |
| C11orf84 | 0.597933597 |
| RERGL | 0.597884075 |
| DEFB106B | 0.597385746 |
| FOCAD | 0.597194616 |
| ABHD15 | 0.596915699 |
| MAT1A | 0.596830997 |
| PLA2G16 | 0.596359609 |
| LOC613037 | 0.596353679 |
| ZNF22 | 0.596256075 |
| SERPINF1 | 0.596083127 |
| LOC100130880 | 0.595838656 |
| EIF2B4 | 0.595777423 |
| PCDH18 | 0.595463458 |
| TOR1B | 0.595463391 |
| EVC | 0.595334299 |
| ZC3H7B | 0.594990593 |
| DHRS7 | 0.594853808 |
| CCT8L2 | 0.594839478 |
| HS1BP3 | 0.594559455 |
| GOLGA6L1 | 0.594516541 |
| SNX17 | 0.594415636 |
| AGO4 | 0.594072325 |
| GPAA1 | 0.593771171 |
| ILF2 | 0.593667449 |
| CYB5R1 | 0.593531145 |
| TMEM198 | 0.593254381 |
| MYRIP | 0.593163749 |
| HIST1H3G | 0.593087052 |
| ECE2 | 0.592082021 |
| CTU1 | 0.59180686 |
| CORT | 0.591782038 |
| SYT16 | 0.591722616 |
| KLHDC7B | 0.591545202 |
| MRLN | 0.591462121 |
| MAN2A1 | 0.591206188 |
| SPON2 | 0.590687944 |
| GDF11 | 0.590633196 |
| EYA1 | 0.590526924 |
| CLEC1A | 0.590154143 |
| NOP2 | 0.590151709 |
| TEX30 | 0.590148718 |
| NMRK2 | 0.589800744 |
| MAS1 | 0.589319746 |
| SSH2 | 0.588894234 |
| INPP5K | 0.588843493 |
| FAM188B | 0.588743301 |
| LRRC73 | 0.588215492 |
| GPANK1 | 0.587563643 |
| ACTN1 | 0.587347603 |
| MRPL28 | 0.586181592 |
| DHH | 0.585733102 |
| SLC16A7 | 0.585672141 |
| CARD19 | 0.585353372 |
| TOP2A | 0.585182629 |
| ALG3 | 0.585007998 |
| PRELID1 | 0.584938497 |
| SEC24D | 0.584771692 |
| COMT | 0.584728712 |
| SULT1E1 | 0.584567929 |
| CUL1 | 0.584467101 |
| UBA7 | 0.584168111 |
| BCS1L | 0.583940166 |
| ANGPT2 | 0.583870916 |
| CHL1 | 0.583750481 |
| JMJD8 | 0.583715835 |
| NXT1 | 0.583650336 |
| GOLPH3 | 0.583604201 |
| DZIP1 | 0.583436058 |
| GTPBP2 | 0.583288635 |
| NDUFA7 | 0.583121688 |
| INHBB | 0.582990999 |
| TSGA13 | 0.582864257 |
| MAFF | 0.582855773 |
| CTNNA3 | 0.582454813 |
| SH2B3 | 0.582297232 |
| NAT9 | 0.582251021 |
| XIRP2 | 0.581610102 |
| NUDCD3 | 0.581550597 |
| PRAMEF4 | 0.581526839 |
| B3GAT3 | 0.581473217 |
| C2orf68 | 0.581450503 |
| PPARGC1A | 0.581388696 |
| FILIP1L | 0.58129879 |
| APOF | 0.581256377 |
| DEFB112 | 0.5811976 |
| RND2 | 0.581169674 |
| GPR52 | 0.580997871 |
| OR5R1 | 0.580848799 |
| NPR3 | 0.580765782 |
| NAB1 | 0.580719658 |
| CLDN25 | 0.580261239 |
| C15orf39 | 0.580129973 |
| RNF138 | 0.579993033 |
| B9D1 | 0.579776891 |
| ARMC6 | 0.579747361 |
| C10orf95 | 0.579703083 |
| GLS | 0.579279759 |
| NOP14 | 0.579123661 |
| CCDC189 | 0.578517655 |
| FIGN | 0.577715834 |
| PTPN3 | 0.577332055 |
| MSANTD3-TMEFF1 | 0.57733146 |
| ASCL3 | 0.57708795 |
| METTL22 | 0.577072448 |
| OSBPL9 | 0.577065348 |
| ANKZF1 | 0.576805701 |
| CALCOCO2 | 0.57680104 |
| C2orf80 | 0.576638976 |
| SMIM19 | 0.575943982 |
| SOGA3 | 0.575870621 |
| C8orf4 | 0.575856854 |
| KRT38 | 0.575500169 |
| AVIL | 0.57532688 |
| SENP5 | 0.57515056 |
| FOXJ3 | 0.574722699 |
| OR2J3 | 0.574636526 |
| ARHGEF17 | 0.574477203 |
| HOPX | 0.574397627 |
| KLF11 | 0.57423195 |
| GDF5 | 0.574150974 |
| RALBP1 | 0.574118582 |
| FAM177B | 0.573978585 |
| CAD | 0.573906903 |
| ACTR5 | 0.573656373 |
| PPM1J | 0.573656065 |
| GPM6A | 0.573388566 |
| KRT19 | 0.573366192 |
| CLIC3 | 0.573282929 |
| ERGIC3 | 0.573190738 |
| PTPRK | 0.57282171 |
| TRIM46 | 0.57274829 |
| C1orf234 | 0.572730034 |
| WNT3 | 0.572716249 |
| ITFG1 | 0.572585 |
| MTIF2 | 0.572401021 |
| LIG1 | 0.572285842 |
| ISLR2 | 0.572259185 |
| TSTA3 | 0.572221403 |
| TPBG | 0.572199264 |
| GNG3 | 0.571911004 |
| TUBB2A | 0.571612414 |
| ETV5 | 0.571531287 |
| ATP6V0A2 | 0.571382298 |
| TIMM13 | 0.57134155 |
| ANKRD13D | 0.57105477 |
| C8G | 0.570698186 |
| APOM | 0.570631573 |
| S1PR3 | 0.570327054 |
| GFER | 0.570152439 |
| ADRA1A | 0.570149946 |
| FXYD1 | 0.570017771 |
| PKD1 | 0.569621104 |
| S100PBP | 0.569471057 |
| ZSWIM4 | 0.569045034 |
| APBB3 | 0.568980871 |
| NEIL2 | 0.568794027 |
| ARL10 | 0.568660501 |
| CLIP2 | 0.568451041 |
| SERPINB7 | 0.568083807 |
| BCO2 | 0.568034716 |
| SLC38A10 | 0.567784069 |
| DENND6B | 0.567745406 |
| TXN | 0.567492861 |
| NT5E | 0.567455173 |
| SF3A3 | 0.567262934 |
| FAM174A | 0.567248508 |
| ZKSCAN4 | 0.567202334 |
| CBX6 | 0.566986169 |
| GSX2 | 0.56671587 |
| PRH2 | 0.566550716 |
| MECOM | 0.56634919 |
| NOTCH4 | 0.566240553 |
| SNAPC5 | 0.566007803 |
| SIPA1L2 | 0.565764904 |
| PDE1A | 0.565749428 |
| C9orf170 | 0.56574335 |
| OR6C6 | 0.56566029 |
| CD47 | 0.565656546 |
| DNLZ | 0.565619489 |
| SETD9 | 0.565341704 |
| FABP9 | 0.565259113 |
| UCK1 | 0.564891194 |
| TMEM161A | 0.564678057 |
| C14orf28 | 0.564574991 |
| SLC31A2 | 0.564475538 |
| SLC25A39 | 0.564472914 |
| UCHL1 | 0.564378843 |
| PLSCR5 | 0.56431555 |
| CDIPT | 0.564244844 |
| NRSN2 | 0.564102635 |
| ZNF365 | 0.564075767 |
| GLYR1 | 0.563811795 |
| ATP6V1G3 | 0.563627859 |
| MOCS3 | 0.563487664 |
| MAPK3 | 0.563292442 |
| GTF3A | 0.563248985 |
| IDH1 | 0.563245033 |
| RGPD8 | 0.563236511 |
| BORA | 0.562997508 |
| FOXD1 | 0.562969212 |
| CMTR1 | 0.562508117 |
| APP | 0.562376849 |
| PMPCA | 0.562303558 |
| CKAP2L | 0.562275272 |
| KIAA0895 | 0.561920554 |
| KRT10 | 0.561906428 |
| UGT1A3 | 0.561515774 |
| OR8B3 | 0.561422104 |
| PCID2 | 0.561097342 |
| BFSP2 | 0.560867098 |
| CACNG6 | 0.560751633 |
| GOLGA8M | 0.560637429 |
| NMRAL1 | 0.56057172 |
| ZNF236 | 0.560385943 |
| DCLRE1A | 0.560226286 |
| ZSCAN5A | 0.560214736 |
| DDI1 | 0.560210056 |
| SOX5 | 0.560186363 |
| IRF2BPL | 0.560098769 |
| RAC3 | 0.559982539 |
| CD200R1L | 0.559911425 |
| FOSL1 | 0.559869727 |
| ZNF48 | 0.559808734 |
| ACAD8 | 0.559692331 |
| CEP57 | 0.559638117 |
| CAMTA2 | 0.559315224 |
| NBPF12 | 0.559174243 |
| CCDC54 | 0.559094102 |
| ZNF436 | 0.559006647 |
| KCTD21 | 0.558667846 |
| SMR3A | 0.558398956 |
| CYC1 | 0.558384959 |
| MALL | 0.55837846 |
| FBXW11 | 0.557781233 |
| ZNF514 | 0.557624892 |
| SMIM18 | 0.557591176 |
| FAM129B | 0.557426387 |
| PRPF6 | 0.557312782 |
| OR2T1 | 0.557178935 |
| IL20RB | 0.557067993 |
| TRA2B | 0.556896251 |
| FGL1 | 0.556744435 |
| MAPRE3 | 0.556564213 |
| SEPT7 | 0.556373481 |
| ASIC5 | 0.556290437 |
| TSEN54 | 0.556177772 |
| MYRFL | 0.556138388 |
| WDFY1 | 0.556051323 |
| SETD4 | 0.556051109 |
| PCGF2 | 0.555959459 |
| C12orf49 | 0.555881742 |
| TAXIBP3 | 0.555754834 |
| OR4C45 | 0.555617121 |
| TTLL3 | 0.55547305 |
| CTSZ | 0.555382674 |
| RAB20 | 0.555250719 |
| MAPK7 | 0.555071105 |
| CABLES2 | 0.555068186 |
| RAB13 | 0.554944547 |
| UCN | 0.554943133 |
| KCNG1 | 0.554484223 |
| GPR89B | 0.554470912 |
| ITGA8 | 0.554103006 |
| MMP15 | 0.554043847 |
| OR5L2 | 0.553957606 |
| CAPG | 0.553111058 |
| IL23R | 0.553026935 |
| BCAS4 | 0.552310206 |
| OR5L1 | 0.55227379 |
| CLCA1 | 0.552255467 |
| AMOTL1 | 0.552133214 |
| NDUFV1 | 0.552019665 |
| RNF214 | 0.551827077 |
| C8orf89 | 0.551740532 |
| WNT2 | 0.551187743 |
| PUS10 | 0.551155745 |
| DNAJB5 | 0.550777906 |
| HIF1A | 0.550583066 |
| CDKN2B | 0.550544713 |
| SH3BP1 | 0.550217459 |
| FBLIM1 | 0.550105807 |
| LOC100507507 | 0.550087736 |
| MRPL46 | 0.550081266 |
| CORIN | 0.549877132 |
| AMY1C | 0.549859384 |
| GJA9 | 0.549760165 |
| RB1CC1 | 0.549696592 |
| MORN4 | 0.549593227 |
| ABHD6 | 0.549488677 |
| C2CD4B | 0.549408667 |
| PLCB1 | 0.549263127 |
| RANBP10 | 0.548961901 |
| S100A13 | 0.54890489 |
| ETNK2 | 0.548892712 |
| CCR5 | 0.548820635 |
| ANKAR | 0.548741123 |
| FAM160B1 | 0.548632237 |
| RSU1 | 0.548628683 |
| CAMLG | 0.548206298 |
| FAM46C | 0.548024591 |
| PABPN1 | 0.548021479 |
| CAMK4 | 0.547775679 |
| BIN3 | 0.547763356 |
| BTN2A1 | 0.547592772 |
| FANCD2 | 0.547505749 |
| CDKN3 | 0.547154068 |
| AGTPBP1 | 0.54712638 |
| TBX20 | 0.547061532 |
| COPG1 | 0.546742421 |
| OR5D14 | 0.546144333 |
| PPP6R3 | 0.54602676 |
| COX14 | 0.545897472 |
| FOXRED2 | 0.545490791 |
| ARHGEF15 | 0.545420642 |
| FBXW9 | 0.545316401 |
| PWWP2A | 0.545029441 |
| TROAP | 0.544983139 |
| KCNMB2 | 0.54495055 |
| AURKC | 0.544827392 |
| SCO2 | 0.544799451 |
| TMEM52B | 0.544373242 |
| LACTB2 | 0.544353647 |
| AK1 | 0.543916412 |
| CXXC5 | 0.543789099 |
| RIN1 | 0.543782223 |
| YIF1B | 0.543369083 |
| C7 | 0.543288688 |
| ZP4 | 0.543168425 |
| CCDC88C | 0.543001816 |
| SLCO1B7 | 0.54291902 |
| PRAMEF11 | 0.542661377 |
| CDX1 | 0.542503077 |
| SLC16A3 | 0.542331782 |
| MYCT1 | 0.542211575 |
| MCMDC2 | 0.542030485 |
| RANBP3 | 0.541821668 |
| DHODH | 0.541679863 |
| ZP3 | 0.541626293 |
| MLKL | 0.541338511 |
| LY6K | 0.541156668 |
| C16orf97 | 0.541064295 |
| C12orf57 | 0.54099026 |
| MC2R | 0.540897684 |
| SERPINB1 | 0.540854085 |
| P2RX2 | 0.540850426 |
| MAP3K14 | 0.540698016 |
| SVBP | 0.540589052 |
| MEI1 | 0.540567935 |
| CEP152 | 0.540468259 |
| PIK3C2G | 0.540368009 |
| SLC20A2 | 0.540332654 |
| NUTF2 | 0.540327329 |
| C18orf8 | 0.540240364 |
| PCDHAC2 | 0.540182063 |
| SMAP1 | 0.540176081 |
| ZNF653 | 0.540092773 |
| ENTPD6 | 0.539710845 |
| SLC39A1 | 0.539411561 |
| DBF4 | 0.539364046 |
| TARBP1 | 0.539352302 |
| PTPRB | 0.5391439 |
| DYNLL2 | 0.539109128 |
| RNGTT | 0.538846562 |
| COL21A1 | 0.538693441 |
| GAA | 0.538598482 |
| FGF14 | 0.538505272 |
| RUSC1 | 0.538486229 |
| G6PC2 | 0.538388501 |
| OR5T1 | 0.538263287 |
| LMAN2 | 0.538009668 |
| DYNC1LI2 | 0.537888699 |
| NUP93 | 0.537881928 |
| ZNF274 | 0.537809439 |
| LOXL2 | 0.537574222 |
| LOC100506422 | 0.53741693 |
| CHRFAM7A | 0.537069132 |
| ESRRG | 0.536879545 |
| C5orf38 | 0.536879357 |
| COL1A2 | 0.53686712 |
| ALDOA | 0.536776212 |
| GIGYF2 | 0.53661446 |
| PDK1 | 0.536604321 |
| ITPRIPL2 | 0.536588353 |
| INPP5J | 0.536400441 |
| KRTAP1-5 | 0.536371329 |
| SCNN1D | 0.535887986 |
| OR4A47 | 0.535795065 |
| MTFR1L | 0.535771542 |
| FAM213B | 0.535621977 |
| MTFMT | 0.535541401 |
| B4GAT1 | 0.535486163 |
| C10orf111 | 0.535467552 |
| TOMM20L | 0.53545357 |
| PI16 | 0.534968781 |
| HES2 | 0.534942806 |
| COLEC10 | 0.534908612 |
| MPND | 0.534844447 |
| EIF3J | 0.534843276 |
| C1QTNF1 | 0.534703074 |
| CLRN3 | 0.534637978 |
| MYH4 | 0.534223959 |
| ANKS1A | 0.533716109 |
| TNNI3K | 0.533444056 |
| OR2M4 | 0.533364336 |
| GPR149 | 0.533161372 |
| PI3 | 0.533127973 |
| KIAA1462 | 0.532997684 |
| SYDE1 | 0.532938594 |
| TADA3 | 0.532688196 |
| IL15 | 0.532642875 |
| HHIPL1 | 0.532495072 |
| HRCT1 | 0.532473642 |
| NEB | 0.531999611 |
| AOC3 | 0.531878187 |
| BCL2L1 | 0.531809544 |
| WDR83 | 0.531762259 |
| AP2A1 | 0.531749989 |
| TUBAL3 | 0.531651177 |
| VHLL | 0.531451717 |
| TRAF4 | 0.531441883 |
| HHLA2 | 0.531440025 |
| NUPR1 | 0.53129574 |
| MUSK | 0.531222988 |
| CCDC87 | 0.530877935 |
| KRBA1 | 0.530781837 |
| NKD2 | 0.5305868 |
| SPATA33 | 0.530467047 |
| PKMYT1 | 0.530360285 |
| ZNF764 | 0.530310192 |
| MRPL41 | 0.529790545 |
| METTL6 | 0.529763649 |
| HSPB8 | 0.529735581 |
| CLCF1 | 0.52962188 |
| AOC2 | 0.529223847 |
| CREB3L2 | 0.529219856 |
| VLDLR | 0.529211942 |
| SLC22A24 | 0.529182574 |
| CAPN10 | 0.52912559 |
| PTRF | 0.529119334 |
| CMKLR1 | 0.529054452 |
| PEBP1 | 0.529053654 |
| RPL18 | 0.528727906 |
| OTULIN | 0.528700995 |
| CCDC124 | 0.528458568 |
| GNPNAT1 | 0.528426423 |
| ACACB | 0.528091413 |
| MAEL | 0.527861573 |
| RNF126 | 0.527738822 |
| NXPH4 | 0.52761494 |
| TMEM208 | 0.527318392 |
| DENND5A | 0.52730787 |
| SLITRK6 | 0.526889237 |
| ANO2 | 0.526791419 |
| CTAGE6 | 0.526756953 |
| IL13 | 0.526644006 |
| LCN12 | 0.526050458 |
| CDH12 | 0.525977899 |
| BRD2 | 0.525880363 |
| AKR1C2 | 0.525398191 |
| NCBP2 | 0.525262116 |
| RNF5 | 0.525108682 |
| LONP1 | 0.52508879 |
| DUSP3 | 0.525080115 |
| TMEM131 | 0.525057841 |
| TMCO3 | 0.524959055 |
| ZNF512B | 0.524835691 |
| CTSV | 0.524817967 |
| LOC101928841 | 0.524789941 |
| SLC15A2 | 0.524775055 |
| CTAGE5 | 0.524417109 |
| DLC1 | 0.524407748 |
| TMEM132C | 0.52436748 |
| CORO1C | 0.524362004 |
| CASC10 | 0.524214101 |
| ESYT3 | 0.524209113 |
| GART | 0.524179015 |
| MLST8 | 0.524092173 |
| SPINK9 | 0.523963933 |
| ZKSCAN5 | 0.52392531 |
| LBP | 0.523897334 |
| ERVFRD-1 | 0.523879798 |
| NUS1 | 0.523677759 |
| TMEM123 | 0.523671778 |
| ZNF132 | 0.523486418 |
| CAMK2N2 | 0.523456598 |
| HOXB3 | 0.523392077 |
| CHST3 | 0.523391379 |
| RNF170 | 0.523304383 |
| ATF1 | 0.523188273 |
| PCDHA9 | 0.523153353 |
| STX19 | 0.523115427 |
| ARHGEF26 | 0.523095699 |
| CYB5D2 | 0.522989337 |
| ZFYVE19 | 0.522963404 |
| OR7D2 | 0.522917009 |
| ASS1 | 0.52286936 |
| KCTD14 | 0.522471674 |
| ABI3BP | 0.522399364 |
| RNASE4 | 0.522389802 |
| OR52H1 | 0.522327854 |
| SLITRK1 | 0.522111899 |
| SEPHS2 | 0.522099964 |
| ART1 | 0.521894842 |
| LMO3 | 0.521869618 |
| ARHGAP20 | 0.521837233 |
| SPIC | 0.521738176 |
| SOX9 | 0.521651002 |
| OAZ3 | 0.521457645 |
| GMDS | 0.521315303 |
| WDR34 | 0.521204487 |
| TIMELESS | 0.520656045 |
| KRT12 | 0.520603844 |
| KCNV2 | 0.520540561 |
| TSLP | 0.520394021 |
| SVEP1 | 0.520037652 |
| FABP4 | 0.519698798 |
| SLC8A3 | 0.519477154 |
| MRPL53 | 0.51946579 |
| C6orf132 | 0.519448739 |
| PROCA1 | 0.519429607 |
| LCLAT1 | 0.519391 |
| GDAP2 | 0.519290116 |
| CLDN8 | 0.519242617 |
| SSH1 | 0.518667843 |
| DIDO1 | 0.518636986 |
| TMEM201 | 0.518511288 |
| TMEM52 | 0.518504954 |
| ATP5F1 | 0.51847357 |
| SLC52A3 | 0.518250879 |
| CLDN4 | 0.518242105 |
| TMBIM1 | 0.518106946 |
| ACAD9 | 0.51776819 |
| FAM86C1 | 0.517641105 |
| FOXF1 | 0.517608455 |
| EEF1A2 | 0.517569937 |
| NAA60 | 0.517468608 |
| SCARF2 | 0.517315371 |
| ETV2 | 0.517287278 |
| BAAT | 0.517244868 |
| C4BPB | 0.517192103 |
| CBFB | 0.517152509 |
| SLC25A22 | 0.51710615 |
| OTOP1 | 0.517066907 |
| IL12B | 0.516850473 |
| HDAC11 | 0.51674991 |
| C16orf45 | 0.516608689 |
| FAM213A | 0.516604223 |
| NAT2 | 0.516365528 |
| MPI | 0.516219352 |
| GPR148 | 0.516122223 |
| UBE2Q1 | 0.516006195 |
| BHLHE23 | 0.515615273 |
| PUSL1 | 0.515604059 |
| GPHA2 | 0.515371937 |
| LRP2BP | 0.515360734 |
| OR6V1 | 0.515348873 |
| OR5K4 | 0.515297878 |
| CHRM2 | 0.515153999 |
| SLC16A11 | 0.515097276 |
| LOC730159 | 0.51503336 |
| DNASE1L2 | 0.514951363 |
| PLPP6 | 0.514786594 |
| NAP1L5 | 0.514778727 |
| PCGF5 | 0.514660487 |
| HSD17B1 | 0.514549086 |
| OR6K2 | 0.514119937 |
| NKIRAS1 | 0.514008321 |
| BOLA2-SMG1P6 | 0.513995998 |
| LRRN2 | 0.513886869 |
| TGIF1 | 0.513809274 |
| PLEKHJ1 | 0.513614117 |
| MCM7 | 0.513608366 |
| PCDHB8 | 0.513361532 |
| TP53I11 | 0.513192463 |
| VN1R4 | 0.512986761 |
| NSG1 | 0.512846876 |
| SPINK5 | 0.512801919 |
| HDGF | 0.512682366 |
| ANAPC15 | 0.512660148 |
| CMTM3 | 0.512592955 |
| C3orf79 | 0.512491244 |
| PLPPR2 | 0.511864037 |
| TBC1D31 | 0.511621447 |
| ZBTB49 | 0.511560205 |
| RTP5 | 0.511408846 |
| DACT2 | 0.511339503 |
| TEKT3 | 0.511233968 |
| CDCA5 | 0.511114319 |
| CHST6 | 0.511108995 |
| DMBT1 | 0.510940732 |
| NCS1 | 0.510901943 |
| RCAN2 | 0.510845023 |
| PURB | 0.510798833 |
| FST | 0.510776328 |
| WWP1 | 0.510720966 |
| TOM1 | 0.510658131 |
| GPR32 | 0.510650161 |
| SSC5D | 0.510530749 |
| NAT1 | 0.51031523 |
| TRPT1 | 0.510067581 |
| C2orf88 | 0.510057536 |
| MARK1 | 0.51003332 |
| SDHA | 0.509885798 |
| CMTM6 | 0.509661111 |
| SAFB | 0.509567144 |
| COL17A1 | 0.509356884 |
| KAT14 | 0.509308484 |
| GRK7 | 0.508999292 |
| PCDHA4 | 0.508560164 |
| CDH18 | 0.508534557 |
| SNIP1 | 0.508331031 |
| NPM1 | 0.508267312 |
| KRT40 | 0.508093087 |
| GALNT5 | 0.508080185 |
| ACVRL1 | 0.507713192 |
| ERBB2 | 0.507588465 |
| MARC2 | 0.50755239 |
| HHLA1 | 0.507517934 |
| ARSA | 0.507420148 |
| RAD23A | 0.507343507 |
| B3GAT2 | 0.507099475 |
| CXCL12 | 0.507012825 |
| COLGALT2 | 0.506953459 |
| ZSCAN10 | 0.506952617 |
| TALDO1 | 0.506890491 |
| SGCZ | 0.506873827 |
| RCN2 | 0.506787181 |
| OR10X1 | 0.506561703 |
| OR52I1 | 0.506558623 |
| EGLN1 | 0.506362914 |
| PCDHB7 | 0.50628122 |
| PRR15 | 0.506177642 |
| ZDHHC6 | 0.506071073 |
| HOXD13 | 0.506047477 |
| SERTAD3 | 0.505913285 |
| ALDH16A1 | 0.505865084 |
| CPLX4 | 0.505855062 |
| E2F6 | 0.505642053 |
| CDH1 | 0.505501756 |
| PIEZO2 | 0.505384382 |
| USP54 | 0.505356442 |
| CYYR1 | 0.505346503 |
| GATS | 0.505266789 |
| TACC2 | 0.505253586 |
| SYN3 | 0.505188224 |
| NRF1 | 0.505058424 |
| SPATA2 | 0.505053164 |
| SLC25A26 | 0.504660905 |
| RER1 | 0.504176062 |
| SLC39A12 | 0.503966071 |
| MTRNR2L6 | 0.503914686 |
| INO80B | 0.503847646 |
| ZIC2 | 0.503593014 |
| NOXA1 | 0.503497859 |
| PAAF1 | 0.503481785 |
| IRX1 | 0.503187137 |
| SYT13 | 0.502662095 |
| CSTL1 | 0.502588205 |
| IQCA1 | 0.502415691 |
| ALX4 | 0.502350639 |
| CARMIL2 | 0.502097786 |
| KATNA1 | 0.502059161 |
| SLC35F6 | 0.501999098 |
| SERTM1 | 0.501958541 |
| TCEB2 | 0.501902864 |
| PRDM12 | 0.501666783 |
| TMEM265 | 0.501658246 |
| FAM101A | 0.501602707 |
| SIX3 | 0.501450255 |
| CSN1S1 | 0.50126736 |
| REP15 | 0.501120522 |
| ARSJ | 0.500974917 |
| GPR162 | 0.500838262 |
| PLCXD3 | 0.500802755 |
| CEP128 | 0.500617005 |
| COL3A1 | 0.500594087 |
| EPHA6 | 0.500385155 |
| PTPRE | 0.500030107 |

TABLE 5
Gene Panel: Target Genes

| Target Gene Names | Target Gene Names | Target Gene Names |
|---|---|---|
| A1BG/NM130786.3 | GLO1/NM006708.2 | PRDM16/NM022114.3 |
| A1CF/NM138932.2 | GLOD4/NM016080.3 | PRDM2/NM012231.4 |
| A2M/NM000014.4 | GLP1R/NM002062.4 | PRDM4/NM012406.3 |
| A2ML1/NM144670.5 | GLRA1/NM000171.3 | PRDM5/NM018699.3 |
| A3GALT2/NM001080438.1 | GLRB/NM000824.4 | PRDM7/NM001098173.1 |
| A4GALT/NM001318038.1 | GLRB/NM001166060.1 | PRDM8/NM001099403.1 |
| A4GALT/NM017436.5 | GLRX/NM001243659.1 | PRDM8/NM020226.3 |
| A4GNT/NM016161.2 | GLRX2/NM016066.4 | PRDM9/NM020227.3 |
| AAAS/NM015665.5 | GLRX3/NM006541.4 | PRDX1/NM001202431.1 |
| AACS/NM023928.4 | GLRX5/NM016417.2 | PRDX1/NM002574.3 |
| AADACL2/NM207365.3 | GLS/NM014905.4 | PRDX2/NM005809.5 |
| AADACL4/NM001013630.1 | GLS2/NM013267.3 | PRDX3/NM006793.4 |
| AADAT/NM001286683.1 | GLT6D1/NM182974.2 | PRDX5/NM012094.4 |
| AADAT/NM016228.3 | GLT8D1/NM001010983.2 | PRDX6/NM004905.2 |
| AADAT/NM182662.1 | GLT8D1/NM152932.2 | PREB/NM013388.4 |
| AAED1/NM153698.1 | GLT8D2/NM031302.4 | PRELID1/NM013237.3 |
| AAGAB/NM024666.4 | GLTP/NM016433.3 | PRELID2/NM205846.2 |
| AAK1/NM014911.3 | GLTPD2/NM001014985.2 | PRELID3A/NM006553.3 |
| AAMP/NM001087.4 | GLTSCR1/NM015711.3 | PRELID3B/NM016045.2 |
| AANAT/NM001088.2 | GLTSCR1L/NM001318819.1 | PRELP/NM201348.1 |
| AAR2/NM001271874.1 | GLTSCR1L/NM015349.2 | PREP/NM002726.4 |
| AARS/NM001605.2 | GLTSCR2/NM015710.4 | PREPL/NM001171603.1 |
| AARS2/NM020745.3 | GLUD1/NM005271.4 | PREPL/NM001171606.1 |
| AARSD1/NM001261434.1 | GLUL/NM001033056.3 | PREPL/NM006036.4 |
| AASDH/NM181806.3 | GLUL/NM002065.6 | PREX1/NM020820.3 |
| AASDHPPT/NM015423.2 | GLYATL1/NM080661.4 | PREX2/NM024870.3 |
| AASS/NM005763.3 | GLYATL2/NM145016.3 | PRF1/NM001083116.1 |
| AATF/NM012138.3 | GLYATL3/NM001010904.1 | PRG2/NM001302927.1 |
| AATK/NM001080395.2 | GLYCTK/NM145262.3 | PRG3/NM006093.3 |
| ABAT/NM000663.4 | GLYR1/NM032569.3 | PRG4/NM005807.4 |
| ABAT/NM001127448.1 | GM2A/NM000405.4 | PRH1/NM001291314.1 |
| ABAT/NM020686.5 | GMCL1/NM178439.4 | PRH2/NM001110213.1 |
| ABCA10/NM080282.3 | GMDS/NM001500.3 | PRICKLE1/NM001144881.1 |
| ABCA12/NM173076.2 | GMEB1/NM006582.3 | PRICKLE1/NM001144882.1 |
| ABCA13/NM152701.4 | GMEB2/NM012384.4 | PRICKLE1/NM153026.2 |
| ABCA2/NM001606.4 | GMFG/NM004877.3 | PRICKLE2/NM198859.3 |
| ABCA3/NM001089.2 | GMIP/NM016573.3 | PRICKLE4/NM013397.5 |
| ABCA4/NM000350.2 | GML/NM002066.2 | PRIM1/NM000946.2 |
| ABCA5/NM018672.4 | GMPPA/NM013335.3 | PRIM2/NM000947.4 |
| ABCA5/NM172232.3 | GMPPA/NM205847.2 | PRIMA1/NM178013.3 |
| ABCA6/NM080284.2 | GMPPB/NM021971.2 | PRIMPOL/NM152683.3 |
| ABCA7/NM019112.3 | GMPR/NM006877.3 | PRKAA1/NM006251.5 |
| ABCA8/NM001288985.1 | GMPR2/NM001002001.2 | PRKAA2/NM006252.3 |
| ABCA9/NM080283.3 | GMPR2/NM001002002.2 | PRKAB1/NM006253.4 |
| ABCB1/NM000927.4 | GMPS/NM003875.2 | PRKAB2/NM005399.4 |
| ABCB10/NM012089.2 | GNA11/NM002067.4 | PRKACA/NM002730.3 |
| ABCB11/NM003742.2 | GNA12/NM007353.2 | PRKACB/NM182948.3 |
| ABCB4/NM018849.2 | GNA13/NM006572.5 | PRKACG/NM002732.3 |
| ABCB5/NM001163941.1 | GNA14/NM004297.3 | PRKAG1/NM002733.4 |
| ABCB6/NM005689.2 | GNA15/NM002068.3 | PRKAG2/NM016203.3 |
| ABCB8/NM007188.4 | GNAI1/NM002069.5 | PRKAR1A/NM001278433.1 |
| ABCB9/NM019625.3 | GNAI2/NM002070.3 | PRKAR1A/NM002734.4 |
| ABCC1/NM004996.3 | GNAI3/NM006496.3 | PRKAR1A/NM212471.2 |
| ABCC10/NM033450.2 | GNAL/NM001261443.1 | PRKAR1B/NM001164758.1 |
| ABCC11/NM032583.3 | GNAO1/NM020988.2 | PRKAR1B/NM001164760.1 |
| ABCC11/NM033151.3 | GNAQ/NM002072.4 | PRKAR1B/NM001164761.1 |
| ABCC12/NM033226.2 | GNAS/NM000516.5 | PRKAR1B/NM001164762.1 |
| ABCC2/NM000392.4 | GNAT2/NM005272.3 | PRKAR1B/NM002735.2 |
| ABCC3/NM003786.3 | GNAT3/NM001102386.2 | PRKAR2A/NM004157.3 |
| ABCC4/NM005845.4 | GNAZ/NM002073.2 | PRKAR2B/NM002736.2 |
| ABCC5/NM005688.3 | GNB1/NM002074.4 | PRKCA/NM002737.2 |
| ABCC6/NM001171.5 | GNB2/NM005273.3 | PRKCB/NM002738.6 |
| ABCC8/NM000352.4 | GNB3/NM002075.3 | PRKCD/NM212539.1 |
| ABCC9/NM005691.3 | GNB4/NM021629.3 | PRKCDBP/NM145040.2 |
| ABCD2/NM005164.3 | GNB5/NM016194.3 | PRKCE/NM005400.2 |
| ABCD3/NM002858.3 | GNE/NM005476.5 | PRKCG/NM002739.4 |
| ABCD4/NM005050.3 | GNG10/NM001198664.1 | PRKCH/NM006255.4 |
| ABCE1/NM001040876.1 | GNG11/NM004126.3 | PRKCQ/NM001323265.1 |
| ABCF1/NM001025091.1 | GNG12/NM018841.5 | PRKCQ/NM006257.4 |
| ABCF2/NM007189.2 | GNG13/NM016541.2 | PRKCSH/NM002743.3 |
| ABCF3/NM018358.2 | GNG2/NM001243774.1 | PRKCZ/NM002744.4 |
| ABCG1/NM016818.2 | GNG3/NM012202.4 | PRKD1/NM002742.2 |
| ABCG2/NM004827.2 | GNG4/NM001098721.1 | PRKD2/NM016457.4 |
| ABCG4/NM001142505.1 | GNG4/NM001098722.1 | PRKD3/NM005813.4 |
| ABCG4/NM022169.4 | GNG5/NM005274.2 | PRKDC/NM006904.6 |
| ABHD1/NM032604.3 | GNG7/NM052847.2 | PRKG1/NM001098512.2 |
| ABHD10/NM018394.3 | GNG8/NM033258.1 | PRKG2/NM006259.2 |
| ABHD12/NM001042472.2 | GNGT1/NM021955.3 | PRKRA/NM003690.4 |
| ABHD12B/NM181814.1 | GNGT2/NM001198754.1 | PRKRIP1/NM024653.3 |
| ABHD13/NM032859.2 | GNGT2/NM001198755.1 | PRL/NM000948.5 |
| ABHD14A/NM015407.4 | GNGT2/NM031498.2 | PRL/NM001163558.2 |
| ABHD14B/NM032750.2 | GNL1/NM005275.3 | PRLH/NM015893.1 |
| ABHD15/NM198147.2 | GNL2/NM013285.2 | PRLHR/NM004248.2 |
| ABHD16A/NM021160.2 | GNL3/NM014366.4 | PRLR/NM000949.6 |
| ABHD16B/NM080622.3 | GNLY/NM006433.4 | PRM1/NM002761.2 |
| ABHD17A/NM001130111.1 | GNMT/NM018960.5 | PRM2/NM002762.3 |
| ABHD17B/NM001025780.2 | GNPAT/NM014236.3 | PRM3/NM021247.2 |
| ABHD17C/NM021214.1 | GNPDA1/NM005471.4 | PRMT1/NM198318.4 |
| ABHD2/NM152924.4 | GNPDA2/NM138335.2 | PRMT2/NM001535.4 |
| ABHD3/NM138340.4 | GNPNAT1/NM198066.3 | PRMT3/NM005788.3 |
| ABHD5/NM016006.4 | GNPTAB/NM024312.4 | PRMT5/NM006109.4 |
| ABHD6/NM020676.6 | GNRH1/NM001083111.1 | PRMT6/NM018137.2 |
| ABHD8/NM024527.4 | GNRH2/NM001501.1 | PRMT7/NM001290018.1 |
| ABI1/NM001012750.2 | GNRHR/NM000406.2 | PRMT7/NM019023.2 |
| ABI2/NM005759.5 | GNS/NM002076.3 | PRMT8/NM019854.4 |
| ABI3/NM016428.2 | GOLGA1/NM002077.3 | PRMT9/NM138364.3 |
| ABI3BP/NM015429.3 | GOLGA2/NM004486.4 | PRND/NM012409.3 |
| ABL1/NM007313.2 | GOLGA3/NM005895.3 | PRNP/NM001080122.1 |
| ABL2/NM007314.3 | GOLGA4/NM002078.4 | PRNP/NM001080123.1 |
| ABLIM1/NM001322886.1 | GOLGA5/NM005113.3 | PROB1/NM001161546.1 |
| ABLIM2/NM001130083.1 | GOLGA6A/NM001038640.2 | PROC/NM000312.3 |
| ABLIM3/NM014945.3 | GOLGA6B/NM018652.4 | PROCA1/NM152465.2 |
| ABO/NM020469.2 | GOLGA6C/NM001164404.1 | PROCR/NM006404.4 |
| ABR/NM021962.4 | GOLGA6D/NM001145224.1 | PRODH/NM016335.4 |
| ABRA/NM139166.4 | GOLGA6L1/NM001001413.3 | PROK1/NM032414.2 |
| ABRACL/NM021243.2 | GOLGA6L10/NM001164465.3 | PROK2/NM021935.3 |
| ABT1/NM013375.3 | GOLGA6L2/NM001304388.1 | PROKR1/NM138964.2 |
| ABTB1/NM172027.2 | GOLGA6L3/NM001310153.1 | PROKR2/NM144773.3 |
| ABTB2/NM145804.2 | GOLGA6L4/NM001267536.2 | PROM1/NM006017.2 |
| ACAA1/NM001607.3 | GOLGA6L6/NM001145004.2 | PROM2/NM001165977.2 |
| ACAA2/NM006111.2 | GOLGA6L9/NM198181.3 | PROP1/NM006261.4 |
| ACACA/NM198836.2 | GOLGA7/NM001002296.1 | PROS1/NM000313.3 |
| ACACA/NM198839.2 | GOLGA7/NM016099.2 | PROSC/NM007198.3 |
| ACACB/NM001093.3 | GOLGA7B/NM001010917.2 | PROSER1/NM025138.4 |
| ACAD10/NM025247.5 | GOLGA8A/NM181077.3 | PROSER2/NM153256.3 |
| ACAD11/NM032169.4 | GOLGA8B/NM001023567.4 | PROSER3/NM001039887.2 |
| ACAD8/NM014384.2 | GOLGA8H/NM001282490.1 | PROX1/NM001270616.1 |
| ACAD9/NM014049.4 | GOLGA8K/NM001282493.1 | PROX1/NM002763.4 |
| ACADM/NM000016.5 | GOLGA8M/NM001282468.1 | PROX2/NM001243007.1 |
| ACADS/NM000017.3 | GOLGA8N/NM001282494.1 | PROZ/NM003891.2 |
| ACADSB/NM001609.3 | GOLGA8R/NM001282484.1 | PRPF18/NM003675.3 |
| ACADVL/NM000018.3 | GOLGB1/NM004487.4 | PRPF3/NM004698.2 |
| ACAP1/NM014716.3 | GOLIM4/NM014498.4 | PRPF31/NM015629.3 |
| ACAP2/NM012287.5 | GOLM1/NM016548.3 | PRPF38B/NM018061.2 |
| ACAP3/NM030649.2 | GOLM1/NM177937.2 | PRPF4/NM004697.4 |
| ACAT1/NM000019.3 | GOLPH3/NM022130.3 | PRPF40A/NM017892.3 |
| ACAT2/NM005891.2 | GOLPH3L/NM018178.5 | PRPF40B/NM001031698.2 |
| ACBD3/NM022735.3 | GOLT1A/NM198447.1 | PRPF4B/NM003913.4 |
| ACBD4/NM001135705.2 | GOLT1B/NM016072.4 | PRPF6/NM012469.3 |
| ACBD5/NM001042473.3 | GON4L/NM001282858.1 | PRPF8/NM006445.3 |
| ACBD6/NM032360.3 | GOPC/NM020399.3 | PRPH/NM006262.3 |
| ACBD7/NM001039844.2 | GORAB/NM152281.2 | PRPH2/NM000322.4 |
| ACCS/NM001127219.1 | GORASP1/NM031899.3 | PRPS1L1/NM175886.2 |
| ACCS/NM032592.3 | GORASP2/NM015530.4 | PRPSAP1/NM002766.2 |
| ACCSL/NM001031854.2 | GOSR1/NM004871.2 | PRPSAP2/NM002767.3 |
| ACE/NM000789.3 | GOSR2/NM004287.4 | PRR11/NM018304.3 |
| ACER1/NM133492.2 | GOT1/NM002079.2 | PRR12/NM020719.1 |
| ACER2/NM001010887.2 | GOT1L1/NM152413.2 | PRR13/NM001005354.2 |
| ACER3/NM018367.6 | GOT2/NM002080.3 | PRR14/NM001320464.1 |
| ACHE/NM000665.4 | GP1BA/NM000173.6 | PRR14/NM024031.3 |
| ACHE/NM001302622.1 | GP1BB/NM000407.4 | PRR14L/NM173566.2 |
| ACIN1/NM014977.3 | GP5/NM004488.2 | PRR15/NM175887.2 |
| ACKR1/NM002036.3 | GP6/NM001083899.2 | PRR15L/NM024320.3 |
| ACKR2/NM001296.4 | GP9/NM000174.4 | PRR16/NM001300783.1 |
| ACKR3/NM020311.2 | GPA33/NM005814.2 | PRR18/NM175922.3 |
| ACKR4/NM016557.3 | GPAA1/NM003801.3 | PRR19/NM199285.2 |
| ACKR4/NM178445.2 | GPALPP1/NM018559.3 | PRR20A/NM198441.2 |
| ACLY/NM001096.2 | GPAM/NM001244949.1 | PRR20B/NM001130404.1 |
| ACMSD/NM138326.2 | GPANK1/NM001199237.1 | PRR20C/NM001130405.1 |
| ACO1/NM002197.2 | GPANK1/NM001199238.1 | PRR20D/NM001130406.1 |
| ACO2/NM001098.2 | GPANK1/NM001199240.1 | PRR20E/NM001130407.1 |
| ACOD1/NM001258406.1 | GPANK1/NM033177.3 | PRR21/NM001080835.1 |
| ACOT1/NM001037161.1 | GPAT2/NM207328.3 | PRR22/NM001134316.1 |
| ACOT11/NM015547.3 | GPAT3/NM001256421.1 | PRR23A/NM001134659.1 |
| ACOT12/NM130767.2 | GPAT3/NM001256422.1 | PRR23B/NM001013650.2 |
| ACOT13/NM018473.3 | GPAT3/NM032717.4 | PRR23C/NM001134657.1 |
| ACOT2/NM006821.5 | GPAT4/NM178819.3 | PRR23D1/NM001282479.1 |
| ACOT4/NM152331.3 | GPATCH2/NM018040.3 | PRR23D2/NM001282478.1 |
| ACOT7/NM181864.2 | GPATCH2L/NM017926.3 | PRR25/NM001013638.1 |
| ACOX1/NM007292.5 | GPATCH3/NM022078.2 | PRR29/NM001191029.1 |
| ACOX2/NM003500.3 | GPATCH4/NM182679.2 | PRR3/NM025263.3 |
| ACOX3/NM003501.2 | GPATCH8/NM001002909.3 | PRR30/NM178553.3 |
| ACP1/NM004300.3 | GPBAR1/NM001321950.1 | PRR34/NM018280.2 |
| ACP2/NM001610.3 | GPBAR1/NM170699.2 | PRR35/NM145270.2 |
| ACP5/NM001111034.2 | GPBP1/NM022913.3 | PRR36/NM001190467.1 |
| ACP5/NM001111035.2 | GPBP1L1/NM021639.4 | PRR4/NM007244.2 |
| ACP5/NM001322023.1 | GPC1/NM002081.2 | PRR5-ARHGAP8/NM181334.5 |
| ACP5/NM001611.4 | GPC2/NM152742.2 | PRR5L/NM001160167.1 |
| ACP6/NM016361.4 | GPC5/NM004466.5 | PRR5L/NM024841.4 |
| ACPP/NM001099.4 | GPC6/NM005708.3 | PRR7/NM001174101.1 |
| ACPT/NM033068.2 | GPCPD1/NM019593.3 | PRR7/NM001174102.1 |
| ACR/NM001097.2 | GPD1/NM005276.3 | PRR9/NM001195571.1 |
| ACRBP/NM032489.2 | GPD1L/NM015141.3 | PRRC1/NM130809.4 |
| ACRV1/NM001612.5 | GPD2/NM000408.4 | PRRC2A/NM080686.2 |
| ACSBG2/NM001289178.1 | GPD2/NM001083112.2 | PRRC2B/NM013318.3 |
| ACSF2/NM025149.5 | GPER1/NM001098201.1 | PRRC2C/NM015172.3 |
| ACSF3/NM174917.4 | GPER1/NM001505.2 | PRRG2/NM000951.2 |
| ACSL1/NM001286708.1 | GPHA2/NM130769.3 | PRRG4/NM024081.5 |
| ACSL1/NM001995.3 | GPHB5/NM145171.3 | PRRT1/NM030651.3 |
| ACSL3/NM203372.1 | GPHN/NM020806.4 | PRRT2/NM145239.2 |
| ACSL5/NM203379.1 | GPI/NM000175.3 | PRRT3/NM207351.4 |
| ACSL5/NM203380.1 | GPIHBP1/NM178172.5 | PRRT4/NM001114726.2 |
| ACSL6/NM001009185.2 | GPLD1/NM001503.3 | PRRX1/NM022716.3 |
| ACSM1/NM001318890.1 | GPM6A/NM005277.4 | PRRX2/NM016307.3 |
| ACSM1/NM052956.2 | GPM6A/NM201591.2 | PRSS1/NM002769.4 |
| ACSM2A/NM001308172.1 | GPN1/NM007266.3 | PRSS12/NM003619.3 |
| ACSM2B/NM182617.3 | GPN2/NM018066.3 | PRSS16/NM005865.3 |
| ACSM3/NM005622.3 | GPR1/NM001261453.1 | PRSS2/NM002770.3 |
| ACSM4/NM001080454.1 | GPR107/NM020960.4 | PRSS21/NM006799.3 |
| ACSM5/NM017888.2 | GPR108/NM001080452.1 | PRSS22/NM022119.3 |
| ACSM6/NM207321.2 | GPR12/NM005288.3 | PRSS23/NM001293179.1 |
| ACSS1/NM032501.3 | GPR132/NM001278694.1 | PRSS23/NM001293180.1 |
| ACSS2/NM018677.3 | GPR135/NM022571.5 | PRSS27/NM031948.4 |
| ACSS3/NM024560.2 | GPR137/NM001170880.1 | PRSS3/NM002771.3 |
| ACTA1/NM001100.3 | GPR137B/NM003272.3 | PRSS33/NM152891.2 |
| ACTA2/NM001141945.2 | GPR137C/NM001099652.1 | PRSS35/NM153362.2 |
| ACTA2/NM001613.2 | GPR139/NM001002911.3 | PRSS36/NM173502.4 |
| ACTBL2/NM001017992.3 | GPR141/NM181791.1 | PRSS37/NM001008270.2 |
| ACTC1/NM005159.4 | GPR142/NM181790.1 | PRSS38/NM183062.2 |
| ACTG1/NM001199954.1 | GPR146/NM001303473.1 | PRSS41/NM001135086.1 |
| ACTG2/NM001615.3 | GPR146/NM138445.3 | PRSS42/NM182702.1 |
| ACTL10/NM001024675.1 | GPR148/NM207364.2 | PRSS45/NM199183.2 |
| ACTL6A/NM004301.4 | GPR149/NM001038705.1 | PRSS46/NM001205271.1 |
| ACTL6B/NM016188.4 | GPR15/NM005290.3 | PRSS48/NM183375.2 |
| ACTL7A/NM006687.3 | GPR150/NM199243.1 | PRSS50/NM013270.4 |
| ACTL7B/NM006686.3 | GPR151/NM194251.2 | PRSS53/NM001039503.2 |
| ACTL8/NM030812.2 | GPR152/NM206997.1 | PRSS54/NM001080492.1 |
| ACTL9/NM178525.4 | GPR153/NM207370.2 | PRSS55/NM198464.3 |
| ACTN1/NM001102.3 | GPR155/NM001033045.3 | PRSS56/NM001195129.1 |
| ACTN2/NM001103.3 | GPR156/NM153002.2 | PRSS57/NM214710.4 |
| ACTN3/NM001104.3 | GPR157/NM024980.4 | PRSS58/NM001001317.4 |
| ACTN4/NM004924.5 | GPR158/NM020752.2 | PRSS8/NM002773.4 |
| ACTR10/NM018477.2 | GPR160/NM014373.2 | PRTFDC1/NM020200.6 |
| ACTR1A/NM005736.3 | GPR161/NM001267610.1 | PRTG/NM173814.5 |
| ACTR1B/NM005735.3 | GPR161/NM153832.2 | PRTN3/NM002777.3 |
| ACTR3/NM005721.4 | GPR162/NM019858.1 | PRUNE2/NM015225.2 |
| ACTR3B/NM020445.5 | GPR17/NM001161417.1 | PRX/NM020956.2 |
| ACTR3C/NM001164458.1 | GPR171/NM013308.3 | PSAP/NM002778.3 |
| ACTR3C/NM001164459.1 | GPR176/NM007223.2 | PSAPL1/NM001085382.1 |
| ACTR5/NM024855.3 | GPR179/NM001004334.3 | PSAT1/NM058179.3 |
| ACTR8/NM022899.4 | GPR18/NM005292.3 | PSCA/NM005672.4 |
| ACTRT2/NM080431.4 | GPR180/NM180989.5 | PSD/NM001270965.1 |
| ACTRT3/NM032487.4 | GPR182/NM007264.3 | PSD/NM002779.4 |
| ACVR1/NM001105.4 | GPR183/NM004951.4 | PSD2/NM032289.2 |
| ACVR1/NM001111067.2 | GPR19/NM006143.2 | PSD3/NM206909.2 |
| ACVR1B/NM004302.4 | GPR20/NM005293.2 | PSD4/NM012455.2 |
| ACVR1C/NM145259.2 | GPR21/NM005294.2 | PSEN1/NM000021.3 |
| ACVR2A/NM001278579.1 | GPR22/NM005295.2 | PSEN2/NM000447.2 |
| ACVR2A/NM001616.4 | GPR25/NM005298.3 | PSG2/NM031246.3 |
| ACVR2B/NM001106.3 | GPR26/NM153442.3 | PSG3/NM021016.3 |
| ACVRL1/NM000020.2 | GPR27/NM018971.1 | PSG6/NM002782.4 |
| ACVRL1/NM001077401.1 | GPR3/NM005281.3 | PSG7/NM002783.2 |
| ACY1/NM001198895.1 | GPR31/NM005299.2 | PSIP1/NM033222.4 |
| ACY3/NM080658.1 | GPR32/NM001506.2 | PSKH1/NM006742.2 |
| ACYP1/NM001107.4 | GPR33/NM001197184.2 | PSKH2/NM033126.1 |
| ACYP2/NM138448.3 | GPR35/NM005301.3 | PSMA1/NM002786.3 |
| ADA/NM000022.3 | GPR37/NM005302.3 | PSMA2/NM002787.4 |
| ADAD2/NM139174.3 | GPR37L1/NM004767.3 | PSMA3/NM002788.3 |
| ADAL/NM001159280.2 | GPR39/NM001508.2 | PSMA4/NM001102667.1 |
| ADAM10/NM001110.3 | GPR4/NM005282.2 | PSMA5/NM002790.3 |
| ADAM11/NM002390.5 | GPR45/NM007227.3 | PSMA6/NM002791.2 |
| ADAM12/NM003474.5 | GPR52/NM005684.4 | PSMA7/NM002792.3 |
| ADAM15/NM207197.2 | GPR55/NM005683.3 | PSMA8/NM144662.2 |
| ADAM19/NM033274.4 | GPR6/NM005284.4 | PSMB1/NM002793.3 |
| ADAM2/NM001464.4 | GPR61/NM031936.4 | PSMB10/NM002801.3 |
| ADAM20/NM003814.4 | GPR62/NM080865.3 | PSMB11/NM001099780.1 |
| ADAM21/NM003813.3 | GPR63/NM001143957.2 | PSMB2/NM002794.4 |
| ADAM22/NM021722.5 | GPR65/NM003608.3 | PSMB3/NM002795.3 |
| ADAM23/NM003812.3 | GPR68/NM001177676.1 | PSMB4/NM002796.2 |
| ADAM28/NM014265.5 | GPR68/NM003485.3 | PSMB5/NM002797.4 |
| ADAM30/NM021794.3 | GPR75-ASB3/NM001164165.1 | PSMB7/NM002799.3 |
| ADAM32/NM145004.6 | GPR78/NM080819.4 | PSMB8/NM148919.3 |
| ADAM33/NM025220.3 | GPR83/NM016540.3 | PSMB9/NM002800.4 |
| ADAM7/NM003817.3 | GPR84/NM020370.2 | PSMC1/NM002802.2 |
| ADAM9/NM003816.2 | GPR85/NM001146265.1 | PSMC2/NM002803.3 |
| ADAMDEC1/NM014479.3 | GPR85/NM001146266.1 | PSMC3/NM002804.4 |
| ADAMTS1/NM006988.4 | GPR85/NM001146267.1 | PSMC3IP/NM016556.3 |
| ADAMTS10/NM030957.3 | GPR87/NM023915.3 | PSMC4/NM006503.3 |
| ADAMTS13/NM139025.4 | GPR88/NM022049.2 | PSMC5/NM002805.5 |
| ADAMTS14/NM139155.2 | GPR89A/NM001097612.1 | PSMC6/NM002806.3 |
| ADAMTS15/NM139055.2 | GPR89B/NM016334.3 | PSMD1/NM002807.3 |
| ADAMTS16/NM139056.2 | GPRC5A/NM003979.3 | PSMD11/NM002815.3 |
| ADAMTS17/NM139057.2 | GPRC5B/NM016235.2 | PSMD12/NM002816.4 |
| ADAMTS18/NM199355.2 | GPRC5C/NM022036.2 | PSMD13/NM002817.3 |
| ADAMTS2/NM014244.4 | GPRC6A/NM148963.3 | PSMD14/NM005805.5 |
| ADAMTS20/NM025003.3 | GPRIN1/NM052899.2 | PSMD2/NM002808.4 |
| ADAMTS3/NM014243.2 | GPRIN2/NM014696.3 | PSMD3/NM002809.3 |
| ADAMTS4/NM005099.5 | GPRIN3/NM198281.2 | PSMD5/NM005047.3 |
| ADAMTS5/NM007038.4 | GPS1/NM212492.2 | PSMD6/NM014814.2 |
| ADAMTS6/NM197941.3 | GPSM1/NM001145638.2 | PSMD7/NM002811.4 |
| ADAMTS7/NM014272.4 | GPSM3/NM001276501.1 | PSMD8/NM002812.4 |
| ADAMTS8/NM007037.5 | GPSM3/NM022107.2 | PSMD9/NM002813.6 |
| ADAMTS9/NM182920.1 | GPT/NM005309.2 | PSME1/NM006263.3 |
| ADAMTSL1/NM001040272.5 | GPT2/NM133443.3 | PSME2/NM002818.2 |
| ADAMTSL2/NM001145320.1 | GPX1/NM000581.2 | PSME3/NM005789.3 |
| ADAMTSL2/NM014694.3 | GPX2/NM002083.3 | PSME4/NM014614.2 |
| ADAMTSL3/NM207517.2 | GPX3/NM002084.3 | PSMF1/NM006814.4 |
| ADAMTSL4/NM019032.5 | GPX4/NM002085.4 | PSMF1/NM178578.3 |
| ADAMTSL5/NM213604.2 | GPX5/NM001509.2 | PSMG1/NM003720.3 |
| ADAP1/NM006869.3 | GPX6/NM182701.1 | PSMG2/NM020232.4 |
| ADAP2/NM018404.2 | GPX7/NM015696.4 | PSMG3/NM001134340.1 |
| ADAR/NM001111.4 | GPX8/NM001008397.3 | PSMG3/NM032302.3 |
| ADARB1/NM001112.3 | GRAMD1A/NM020895.4 | PSMG4/NM001128591.1 |
| ADARB2/NM018702.3 | GRAMD1C/NM017577.4 | PSORS1C1/NM014068.2 |
| ADAT1/NM001324445.1 | GRAMD2/NM001012642.2 | PSORS1C2/NM014069.2 |
| ADAT2/NM182503.2 | GRAMD3/NM023927.2 | PSPC1/NM001042414.2 |
| ADAT3/NM138422.2 | GRAMD4/NM015124.4 | PSPH/NM004577.3 |
| ADCK1/NM020421.3 | GRAP/NM006613.3 | PSPN/NM004158.2 |
| ADCK2/NM052853.3 | GRAP2/NM001291824.1 | PSRC1/NM001032291.2 |
| ADCK5/NM174922.4 | GRAP2/NM001291825.1 | PSTK/NM153336.2 |
| ADCY1/NM021116.2 | GRAP2/NM004810.3 | PSTPIP1/NM003978.4 |
| ADCY10/NM018417.5 | GRAPL/NM001129778.1 | PSTPIP2/NM024430.3 |
| ADCY2/NM020546.2 | GRASP/NM181711.3 | PTAFR/NM001164721.1 |
| ADCY3/NM004036.4 | GRB10/NM005311.4 | PTAFR/NM001164723.2 |
| ADCY4/NM001198568.1 | GRB14/NM004490.2 | PTBP1/NM031991.3 |
| ADCY5/NM183357.2 | GRB2/NM002086.4 | PTBP2/NM021190.3 |
| ADCY6/NM015270.4 | GRB7/NM001030002.2 | PTBP3/NM005156.6 |
| ADCY7/NM001114.4 | GRB7/NM001242443.1 | PTCD1/NM015545.3 |
| ADCY8/NM001115.2 | GRB7/NM005310.3 | PTCD2/NM024754.4 |
| ADCY9/NM001116.3 | GREB1/NM014668.3 | PTCD3/NM017952.5 |
| ADCYAP1/NM001099733.1 | GREB1L/NM001142966.1 | PTCH1/NM000264.3 |
| ADCYAP1/NM001117.4 | GREM1/NM013372.6 | PTCH2/NM003738.4 |
| ADCYAP1R1/NM001118.4 | GREM2/NM022469.3 | PTCHD3/NM001034842.3 |
| ADD2/NM001185054.1 | GRHL1/NM198182.2 | PTCRA/NM138296.2 |
| ADD2/NM001617.3 | GRHL2/NM024915.3 | PTDSS1/NM014754.2 |
| ADD3/NM016824.4 | GRHL3/NM198173.2 | PTDSS2/NM030783.1 |
| ADGRA1/NM001083909.2 | GRHPR/NM012203.1 | PTEN/NM000314.6 |
| ADGRA2/NM032777.9 | GRIA1/NM000827.3 | PTF1A/NM178161.2 |
| ADGRA3/NM145290.3 | GRIA2/NM000826.3 | PTGDR/NM000953.2 |
| ADGRB1/NM001702.2 | GRIA4/NM000829.3 | PTGDR2/NM004778.2 |
| ADGRB2/NM001294335.1 | GRID2IP/NM001145118.1 | PTGDS/NM000954.5 |
| ADGRB3/NM001704.2 | GRIFIN/NM001291784.1 | PTGER1/NM000955.2 |
| ADGRD1/NM198827.3 | GRIK1/NM175611.2 | PTGER2/NM000956.3 |
| ADGRE2/NM013447.3 | GRIK2/NM021956.4 | PTGER3/NM198718.1 |
| ADGRE3/NM032571.4 | GRIK3/NM000831.3 | PTGER4/NM000958.2 |
| ADGRE5/NM078481.3 | GRIK4/NM001282470.2 | PTGES/NM004878.4 |
| ADGRF1/NM153840.3 | GRIK4/NM014619.4 | PTGES2/NM025072.6 |
| ADGRF3/NM153835.3 | GRIK5/NM002088.4 | PTGES3/NM006601.6 |
| ADGRF4/NM153838.3 | GRIN1/NM007327.3 | PTGES3L-AARSD1/NM025267.3 |
| ADGRF5/NM001098518.1 | GRIN2A/NM000833.4 | PTGES3L/NM001261430.1 |
| ADGRF5/NM015234.4 | GRIN2A/NM001134407.2 | PTGFR/NM000959.3 |
| ADGRG1/NM001145771.2 | GRIN2B/NM000834.3 | PTGFRN/NM020440.3 |
| ADGRG1/NM005682.6 | GRIN2C/NM000835.4 | PTGIR/NM000960.3 |
| ADGRG3/NM170776.4 | GRIN2D/NM000836.2 | PTGIS/NM000961.3 |
| ADGRG5/NM001304376.2 | GRIN3A/NM133445.2 | PTGR1/NM001146108.1 |
| ADGRG6/NM198569.2 | GRIN3B/NM138690.2 | PTGR1/NM012212.3 |
| ADGRG7/NM032787.2 | GRINA/NM001009184.1 | PTGR2/NM001146154.1 |
| ADGRL1/NM014921.4 | GRIP1/NM021150.3 | PTGR2/NM001146155.1 |
| ADGRL3/NM001322402.1 | GRIP2/NM001080423.3 | PTGR2/NM152444.2 |
| ADGRL4/NM022159.3 | GRK1/NM002929.2 | PTGS1/NM000962.3 |
| ADH1A/NM000667.3 | GRK2/NM001619.3 | PTGS2/NM000963.3 |
| ADH1B/NM000668.5 | GRK3/NM005160.3 | PTH/NM000315.3 |
| ADH1C/NM000669.4 | GRK4/NM182982.2 | PTH1R/NM000316.2 |
| ADH4/NM000670.4 | GRK5/NM005308.2 | PTH1R/NM001184744.1 |
| ADH5/NM000671.4 | GRK6/NM001004106.2 | PTH2/NM178449.3 |
| ADH6/NM000672.3 | GRK7/NM139209.2 | PTH2R/NM005048.3 |
| ADH7/NM000673.4 | GRM2/NM000839.3 | PTHLH/NM002820.2 |
| ADHFE1/NM144650.2 | GRM3/NM000840.2 | PTHLH/NM198964.1 |
| ADI1/NM018269.3 | GRM4/NM000841.3 | PTK2B/NM004103.4 |
| ADIPOQ/NM004797.3 | GRM6/NM000843.3 | PTK2B/NM173174.2 |
| ADIPOR2/NM024551.2 | GRM8/NM000845.2 | PTK2B/NM173176.2 |
| ADIRF/NM006829.2 | GRPEL2/NM152407.3 | PTK6/NM005975.3 |
| ADK/NM006721.3 | GRSF1/NM002092.3 | PTK7/NM002821.4 |
| ADM/NM001124.2 | GRTP1/NM024719.3 | PTMA/NM001099285.1 |
| ADM2/NM001253845.1 | GRWD1/NM031485.3 | PTMS/NM002824.4 |
| ADM5/NM001101340.1 | GRXCR1/NM001080476.2 | PTN/NM001321386.1 |
| ADNP/NM001282531.1 | GRXCR2/NM001080516.1 | PTN/NM002825.5 |
| ADNP/NM001282532.1 | GS1-259H13.2/NM001195543.1 | PTOV1/NM001305105.1 |
| ADNP2/NM014913.3 | GSAP/NM017439.3 | PTP4A1/NM003463.4 |
| ADO/NM032804.5 | GSC/NM173849.2 | PTP4A2/NM080391.3 |
| ADORA1/NM001048230.1 | GSC2/NM005315.1 | PTP4A3/NM032611.2 |
| ADORA2A/NM001278498.1 | GSDMA/NM178171.4 | PTPDC1/NM152422.4 |
| ADORA2A/NM001278499.1 | GSDMB/NM018530.2 | PTPMT1/NM175732.2 |
| ADORA2A/NM001278500.1 | GSDMC/NM031415.2 | PTPN1/NM002827.3 |
| ADORA2B/NM000676.2 | GSDMD/NM001166237.1 | PTPN11/NM002834.3 |
| ADPGK/NM031284.4 | GSDMD/NM024736.6 | PTPN12/NM002835.3 |
| ADPRH/NM001291949.1 | GSE1/NM014615.3 | PTPN13/NM080685.2 |
| ADPRHL1/NM138430.4 | GSG1/NM031289.3 | PTPN14/NM005401.4 |
| ADPRHL2/NM017825.2 | GSG1L/NM001109763.1 | PTPN18/NM014369.3 |
| ADPRM/NM020233.4 | GSG1L2/NM001310219.1 | PTPN2/NM002828.3 |
| ADRA1A/NM000680.3 | GSG2/NM031965.2 | PTPN20/NM001042357.4 |
| ADRA1B/NM000679.3 | GSK3A/NM019884.2 | PTPN21/NM007039.3 |
| ADRA1D/NM000678.3 | GSK3B/NM002093.3 | PTPN22/NM015967.6 |
| ADRA2A/NM000681.3 | GSKIP/NM001271904.1 | PTPN23/NM015466.3 |
| ADRA2B/NM000682.6 | GSKIP/NM001271906.1 | PTPN3/NM002829.3 |
| ADRA2C/NM000683.3 | GSN/NM000177.4 | PTPN4/NM002830.3 |
| ADRB1/NM000684.2 | GSPT1/NM001130007.1 | PTPN5/NM001278238.1 |
| ADRB2/NM000024.5 | GSR/NM000637.3 | PTPN6/NM080548.4 |
| ADRB3/NM000025.2 | GSS/NM000178.3 | PTPN9/NM002833.3 |
| ADSL/NM000026.3 | GSS/NM001322494.1 | PTPRA/NM002836.3 |
| ADSS/NM001126.3 | GSS/NM001322495.1 | PTPRB/NM001109754.2 |
| ADSSL1/NM152328.4 | GSTA1/NM145740.4 | PTPRC/NM002838.4 |
| ADTRP/NM032744.3 | GSTA2/NM000846.4 | PTPRCAP/NM005608.2 |
| AEBP1/NM001129.4 | GSTA4/NM001512.3 | PTPRD/NM002839.3 |
| AEBP2/NM001114176.1 | GSTA5/NM153699.1 | PTPRE/NM006504.5 |
| AEN/NM022767.3 | GSTCD/NM024751.3 | PTPRF/NM002840.3 |
| AES/NM001130.5 | GSTK1/NM015917.2 | PTPRG/NM002841.3 |
| AFAP1/NM198595.2 | GSTM1/NM000561.3 | PTPRH/NM002842.4 |
| AFAP1L1/NM152406.3 | GSTM2/NM000848.3 | PTPRJ/NM002843.3 |
| AFAP1L2/NM001001936.2 | GSTM3/NM000849.4 | PTPRK/NM002844.3 |
| AFF1/NM005935.3 | GSTM4/NM000850.4 | PTPRM/NM002845.3 |
| AFF3/NM001025108.1 | GSTM5/NM000851.3 | PTPRN/NM002846.3 |
| AFF4/NM014423.3 | GSTO1/NM004832.2 | PTPRN2/NM002847.4 |
| AFG3L2/NM006796.2 | GSTO2/NM183239.1 | PTPRO/NM030667.2 |
| AFM/NM001133.2 | GSTT2B/NM001080843.3 | PTPRQ/NM001145026.1 |
| AFMID/NM001010982.4 | GSTZ1/NM145870.2 | PTPRR/NM002849.3 |
| AFP/NM001134.2 | GSX1/NM145657.2 | PTPRS/NM130854.2 |
| AFTPH/NM017657.4 | GSX2/NM133267.2 | PTPRT/NM007050.5 |
| AGA/NM000027.3 | GTDC1/NM001006636.3 | PTPRU/NM005704.4 |
| AGAP1/NM014914.4 | GTF2A1L/NM006872.4 | PTPRZ1/NM002851.2 |
| AGAP11/NM133447.1 | GTF2A2/NM001320929.1 | PTRF/NM012232.5 |
| AGAP2/NM014770.3 | GTF2A2/NM001320930.1 | PTRH1/NM001002913.1 |
| AGAP3/NM031946.6 | GTF2A2/NM004492.2 | PTRH2/NM016077.4 |
| AGAP4/NM133446.3 | GTF2B/NM001514.5 | PTS/NM000317.2 |
| AGAP5/NM001144000.1 | GTF2E2/NM002095.4 | PTTG1/NM001282383.1 |
| AGAP6/NM001077665.2 | GTF2F1/NM002096.2 | PTTG1/NM004219.3 |
| AGAP9/NM001190810.1 | GTF2F2/NM004128.2 | PTTG1IP/NM004339.3 |
| AGBL1/NM152336.2 | GTF2H1/NM005316.3 | PTTG2/NM006607.2 |
| AGBL2/NM024783.3 | GTF2H2/NM001515.3 | PTX3/NM002852.3 |
| AGBL3/NM178563.3 | GTF2H2C/NM001098728.1 | PTX4/NM001013658.1 |
| AGBL4/NM001323575.1 | GTF2H3/NM001516.4 | PUF60/NM078480.2 |
| AGBL5/NM021831.5 | GTF2H4/NM001517.4 | PUM1/NM014676.2 |
| AGER/NM001136.4 | GTF2H5/NM207118.2 | PUM2/NM015317.2 |
| AGFG1/NM004504.4 | GTF2I/NM032999.3 | PUM3/NM014878.4 |
| AGGF1/NM018046.4 | GTF2IRD1/NM016328.2 | PURA/NM005859.4 |
| AGK/NM018238.3 | GTF2IRD2/NM173537.3 | PURB/NM033224.4 |
| AGL/NM000642.2 | GTF2IRD2B/NM001003795.2 | PURG/NM001323311.1 |
| AGL/NM000644.2 | GTF3A/NM002097.2 | PURG/NM013357.2 |
| AGMAT/NM024758.4 | GTF3C1/NM001520.3 | PUS1/NM025215.5 |
| AGMO/NM001004320.1 | GTF3C2/NM001521.3 | PUS10/NM001322123.1 |
| AGO1/NM012199.4 | GTF3C3/NM012086.4 | PUS10/NM001322124.1 |
| AGO2/NM012154.3 | GTF3C4/NM012204.3 | PUS10/NM144709.3 |
| AGO3/NM024852.3 | GTF3C5/NM012087.3 | PUS3/NM031307.3 |
| AGO4/NM017629.3 | GTF3C6/NM138408.3 | PUS7/NM019042.4 |
| AGPAT1/NM006411.3 | GTPBP1/NM004286.4 | PUS7L/NM001098614.2 |
| AGPAT1/NM032741.4 | GTPBP10/NM033107.3 | PUS7L/NM001098615.1 |
| AGPAT2/NM006412.3 | GTPBP2/NM019096.4 | PUS7L/NM031292.4 |
| AGPAT3/NM001037553.1 | GTPBP3/NM133644.3 | PUSL1/NM153339.1 |
| AGPAT3/NM020132.4 | GTPBP4/NM012341.2 | PVALB/NM001315532.1 |
| AGPAT4/NM020133.2 | GTPBP8/NM014170.2 | PVALB/NM002854.2 |
| AGPAT5/NM018361.3 | GTSCR1/NM001278515.1 | PVR/NM006505.4 |
| AGPS/NM003659.3 | GTSE1/NM016426.6 | PVRIG/NM024070.3 |
| AGR2/NM006408.3 | GTSF1/NM144594.2 | PWP1/NM007062.2 |
| AGR3/NM176813.4 | GTSF1L/NM176791.3 | PWP2/NM005049.2 |
| AGRN/NM198576.3 | GUCA1B/NM002098.5 | PWWP2A/NM001130864.1 |
| AGRP/NM001138.1 | GUCA1C/NM005459.3 | PWWP2B/NM138499.3 |
| AGT/NM000029.3 | GUCA2A/NM033553.2 | PXDC1/NM183373.3 |
| AGTPBP1/NM015239.2 | GUCA2B/NM007102.2 | PXDN/NM012293.2 |
| AGTR1/NM009585.3 | GUCD1/NM031444.3 | PXDNL/NM144651.4 |
| AGTRAP/NM020350.4 | GUCY1A2/NM000855.2 | PXK/NM017771.4 |
| AGXT/NM000030.2 | GUCY1A3/NM001130682.2 | PXMP2/NM018663.2 |
| AHCTF1/NM015446.4 | GUCY1A3/NM001130683.3 | PXMP4/NM007238.4 |
| AHCY/NM000687.3 | GUCY1A3/NM001130684.2 | PXN/NM001080855.2 |
| AHCYL1/NM006621.5 | GUCY1A3/NM001256449.1 | PXT1/NM152990.3 |
| AHCYL2/NM015328.3 | GUCY1B3/NM000857.3 | PXYLP1/NM001037172.2 |
| AHDC1/NM001029882.3 | GUCY2C/NM004963.3 | PYCARD/NM013258.4 |
| AHI1/NM001134831.1 | GUCY2D/NM000180.3 | PYCR1/NM001282280.1 |
| AHNAK/NM024060.3 | GUF1/NM021927.2 | PYCR1/NM006907.3 |
| AHNAK2/NM138420.2 | GUK1/NM000858.5 | PYCR2/NM013328.3 |
| AHR/NM001621.4 | GUK1/NM001159391.1 | PYCRL/NM023078.3 |
| AHRR/NM020731.4 | GUK1/NM001242839.1 | PYDC1/NM152901.3 |
| AHSA1/NM012111.2 | GULP1/NM016315.3 | PYDC2/NM001083308.1 |
| AHSP/NM001318221.1 | GUSB/NM000181.3 | PYGB/NM002862.3 |
| AHSP/NM001318222.1 | GVQW2/NM001242740.1 | PYGL/NM002863.4 |
| AICDA/NM020661.2 | GXYLT1/NM173601.1 | PYGM/NM005609.2 |
| AIDA/NM022831.2 | GXYLT2/NM001080393.1 | PYGO2/NM138300.3 |
| AIF1L/NM031426.3 | GYG1/NM004130.3 | PYHIN1/NM152501.4 |
| AIFM2/NM001198696.1 | GYLTL1B/NM001300721.1 | PYM1/NM032345.2 |
| AIFM3/NM144704.2 | GYLTL1B/NM152312.4 | PYROXD1/NM024854.3 |
| AIG1/NM016108.3 | GYPB/NM002100.5 | PYROXD2/NM032709.2 |
| AIM1/NM001624.3 | GYPC/NM002101.4 | PYURF/NM032906.4 |
| AIM1L/NM001039775.3 | GYPE/NM198682.2 | PYY/NM004160.5 |
| AIM2/NM004833.1 | GYS1/NM002103.4 | PZP/NM002864.2 |
| AIMP1/NM001142415.1 | GYS2/NM021957.3 | QDPR/NM000320.2 |
| AIMP1/NM004757.3 | GZF1/NM022482.4 | QKI/NM006775.2 |
| AIMP2/NM006303.3 | GZMA/NM006144.3 | QPCT/NM012413.3 |
| AIP/NM003977.3 | GZMB/NM004131.4 | QPCTL/NM017659.3 |
| AIRE/NM000383.3 | GZMH/NM033423.4 | QPRT/NM014298.4 |
| AJAP1/NM001042478.1 | GZMK/NM002104.2 | QRFP/NM198180.2 |
| AJUBA/NM032876.5 | GZMM/NM005317.3 | QRICH2/NM032134.2 |
| AK1/NM000476.2 | H1F0/NM005318.3 | QRSL1/NM018292.4 |
| AK1/NM001318121.1 | H1FNT/NM181788.1 | QSER1/NM001076786.2 |
| AK2/NM001625.3 | H1FOO/NM153833.2 | QSOX1/NM002826.4 |
| AK4/NM001005353.2 | H1FX/NM006026.3 | QSOX2/NM181701.3 |
| AK4/NM013410.3 | H2AFJ/NM177925.3 | QTRT2/NM024638.3 |
| AK4/NM203464.2 | H2AFV/NM012412.4 | R3HCC1/NM001136108.2 |
| AK5/NM174858.2 | H2AFX/NM002105.2 | R3HCC1L/NM001256620.1 |
| AK6/NM016283.4 | H2AFY/NM138610.2 | R3HDM1/NM015361.3 |
| AK7/NM152327.3 | H2AFY2/NM018649.2 | R3HDML/NM178491.3 |
| AK8/NM152572.2 | H2AFZ/NM002106.3 | RAB10/NM016131.4 |
| AK9/NM001145128.2 | H3F3A/NM002107.4 | RAB11A/NM004663.4 |
| AKAIN1/NM001145194.1 | H3F3B/NM005324.4 | RAB11FIP1/NM001002814.2 |
| AKAP1/NM001242902.1 | H3F3C/NM001013699.2 | RAB11FIP2/NM014904.2 |
| AKAP1/NM001242903.1 | H6PD/NM004285.3 | RAB11FIP3/NM014700.3 |
| AKAP1/NM003488.3 | HAAO/NM012205.2 | RAB11FIP4/NM032932.5 |
| AKAP10/NM007202.3 | HABP2/NM004132.4 | RAB11FIP5/NM015470.2 |
| AKAP11/NM016248.3 | HABP4/NM014282.3 | RAB12/NM001025300.2 |
| AKAP12/NM005100.3 | HACD1/NM014241.3 | RAB13/NM002870.3 |
| AKAP13/NM006738.5 | HACD2/NM198402.3 | RAB14/NM016322.3 |
| AKAP2/NM001198656.1 | HACD3/NM016395.2 | RAB15/NM001308154.1 |
| AKAP3/NM001278309.1 | HACD4/NM001010915.4 | RAB17/NM022449.3 |
| AKAP5/NM004857.3 | HACE1/NM020771.3 | RAB18/NM021252.4 |
| AKAP7/NM016377.3 | HACL1/NM012260.3 | RAB19/NM001008749.2 |
| AKAP8/NM005858.3 | HADH/NM005327.4 | RAB1B/NM030981.2 |
| AKAP8L/NM014371.3 | HADHA/NM000182.4 | RAB20/NM017817.2 |
| AKIP1/NM020642.3 | HADHB/NM000183.2 | RAB22A/NM020673.2 |
| AKIRIN1/NM024595.2 | HAGH/NM005326.4 | RAB23/NM016277.4 |
| AKIRIN2/NM018064.3 | HAGHL/NM001290137.1 | RAB23/NM183227.2 |
| AKNA/NM030767.5 | HAGHL/NM032304.3 | RAB24/NM001031677.3 |
| AKNAD1/NM152763.4 | HAL/NM002108.3 | RAB25/NM020387.3 |
| AKR1A1/NM001202413.1 | HAMP/NM021175.3 | RAB26/NM014353.4 |
| AKR1B1/NM001628.2 | HAND1/NM004821.2 | RAB27A/NM004580.4 |
| AKR1B10/NM020299.4 | HAND2/NM021973.2 | RAB27A/NM183234.2 |
| AKR1C1/NM001353.5 | HAO1/NM017545.2 | RAB27A/NM183235.2 |
| AKR1C2/NM001354.5 | HAO2/NM016527.3 | RAB27A/NM183236.2 |
| AKR1C3/NM003739.5 | HAP1/NM177977.2 | RAB27B/NM004163.4 |
| AKR1C4/NM001818.3 | HAPLN1/NM001884.3 | RAB29/NM003929.2 |
| AKR1E2/NM001040177.2 | HAPLN2/NM021817.2 | RAB2A/NM002865.2 |
| AKR7A2/NM003689.3 | HAPLN3/NM178232.3 | RAB2B/NM032846.3 |
| AKR7A3/NM012067.2 | HAPLN4/NM023002.2 | RAB30/NM001286059.1 |
| AKT1/NM001014432.1 | HARBI1/NM173811.3 | RAB30/NM001286061.1 |
| AKT1/NM005163.2 | HARS/NM002109.5 | RAB30/NM014488.4 |
| AKT1S1/NM001098632.2 | HARS2/NM012208.3 | RAB31/NM006868.3 |
| AKT1S1/NM001278159.1 | HAS1/NM001523.3 | RAB32/NM006834.4 |
| AKTIS1/NM001278160.1 | HAS2/NM005328.2 | RAB33B/NM031296.2 |
| AKT2/NM001626.5 | HAS3/NM001199280.1 | RAB34/NM001256277.1 |
| AKT3/NM005465.4 | HAS3/NM005329.2 | RAB34/NM031934.5 |
| AKTIP/NM001012398.2 | HAT1/NM003642.3 | RAB35/NM006861.6 |
| ALAD/NM000031.5 | HAUS1/NM138443.3 | RAB36/NM004914.3 |
| ALAS1/NM001304443.1 | HAUS2/NM018097.2 | RAB37/NM175738.4 |
| ALB/NM000477.5 | HAUS4/NM001166269.1 | RAB38/NM022337.2 |
| ALCAM/NM001627.3 | HAUS5/NM015302.1 | RAB39A/NM017516.2 |
| ALDH16A1/NM153329.3 | HAUS6/NM017645.4 | RAB3A/NM002866.4 |
| ALDH18A1/NM002860.3 | HAUS8/NM033417.1 | RAB3B/NM002867.3 |
| ALDH1A1/NM000689.4 | HAVCR1/NM001173393.2 | RAB3D/NM004283.3 |
| ALDH1A2/NM003888.3 | HAVCR1/NM012206.3 | RAB3GAP1/NM012233.2 |
| ALDH1A3/NM000693.3 | HAVCR2/NM032782.4 | RAB3GAP2/NM012414.3 |
| ALDH1B1/NM000692.4 | HBA1/NM000558.4 | RAB3IL1/NM013401.3 |
| ALDH1L1/NM012190.3 | HBA2/NM000517.4 | RAB3IP/NM022456.4 |
| ALDH1L2/NM001034173.3 | HBB/NM000518.4 | RAB40B/NM006822.2 |
| ALDH2/NM000690.3 | HBD/NM000519.3 | RAB40C/NM001172663.1 |
| ALDH3A1/NM001135167.1 | HBE1/NM005330.3 | RAB40C/NM001172664.1 |
| ALDH3A1/NM001135168.1 | HBEGF/NM001945.2 | RAB40C/NM001172665.1 |
| ALDH3A2/NM000382.2 | HBG1/NM000559.2 | RAB40C/NM021168.4 |
| ALDH3B1/NM000694.3 | HBG2/NM000184.2 | RAB42/NM001193532.1 |
| ALDH3B1/NM001161473.2 | HBM/NM001003938.3 | RAB43/NM001204883.1 |
| ALDH3B2/NM001031615.1 | HBQ1/NM005331.4 | RAB43/NM001204884.1 |
| ALDH4A1/NM003748.3 | HBS1L/NM006620.3 | RAB43/NM001204885.1 |
| ALDH5A1/NM170740.1 | HBZ/NM005332.2 | RAB43/NM198490.2 |
| ALDH6A1/NM005589.3 | HCAR1/NM032554.3 | RAB44/NM001257357.1 |
| ALDH7A1/NM001182.4 | HCAR2/NM177551.3 | RAB4A/NM004578.3 |
| ALDH8A1/NM022568.3 | HCAR3/NM006018.2 | RAB4B/NM016154.4 |
| ALDH9A1/NM000696.3 | HCFC1R1/NM017885.3 | RAB5A/NM004162.4 |
| ALDOA/NM000034.3 | HCFC2/NM013320.2 | RAB5B/NM001252036.1 |
| ALDOA/NM001127617.2 | HCK/NM002110.3 | RAB5B/NM002868.3 |
| ALDOA/NM184041.2 | HCLS1/NM005335.5 | RAB5C/NM201434.2 |
| ALDOA/NM184043.2 | HCN1/NM021072.3 | RAB6A/NM002869.4 |
| ALDOB/NM000035.3 | HCN2/NM001194.3 | RAB6B/NM016577.3 |
| ALDOC/NM005165.2 | HCN3/NM020897.2 | RAB6C/NM032144.2 |
| ALG1/NM019109.4 | HCN4/NM005477.2 | RAB7A/NM004637.5 |
| ALG10/NM032834.3 | HCRT/NM001524.1 | RAB7B/NM001164522.2 |
| ALG10B/NM001013620.3 | HCRTR1/NM001525.2 | RAB7B/NM177403.5 |
| ALG11/NM001004127.2 | HCRTR2/NM001526.3 | RAB8A/NM005370.4 |
| ALG12/NM024105.3 | HCST/NM014266.3 | RAB8B/NM016530.2 |
| ALG14/NM144988.3 | HDAC1/NM004964.2 | RABAC1/NM006423.2 |
| ALG1L/NM001015050.2 | HDAC10/NM032019.5 | RABEP1/NM004703.5 |
| ALG1L2/NM001136152.1 | HDAC11/NM024827.3 | RABEP2/NM024816.2 |
| ALG2/NM033087.3 | HDAC2/NM001527.3 | RABEPK/NM001174152.1 |
| ALG3/NM005787.5 | HDAC3/NM003883.3 | RABEPK/NM005833.3 |
| ALG5/NM013338.4 | HDAC4/NM006037.3 | RABGAP1/NM012197.3 |
| ALG6/NM013339.3 | HDAC7/NM015401.4 | RABGAP1L/NM014857.4 |
| ALG8/NM024079.4 | HDAC9/NM001321897.1 | RABGEF1/NM014504.2 |
| ALG9/NM001077690.1 | HDC/NM002112.3 | RABGGTA/NM004581.5 |
| ALK/NM004304.4 | HDDC2/NM016063.2 | RABGGTB/NM004582.3 |
| ALKBH2/NM001145374.1 | HDDC3/NM001286451.1 | RABIF/NM002871.4 |
| ALKBH3/NM139178.3 | HDGF/NM004494.2 | RABL2A/NM007082.4 |
| ALKBH4/NM017621.3 | HDGFL1/NM138574.2 | RABL2B/NM001130922.1 |
| ALKBH5/NM017758.3 | HDGFRP2/NM001001520.2 | RABL6/NM024718.4 |
| ALKBH6/NM032878.3 | HDGFRP3/NM016073.3 | RAC1/NM018890.3 |
| ALKBH7/NM032306.3 | HDHD2/NM032124.4 | RAC2/NM002872.4 |
| ALKBH8/NM138775.2 | HDHD3/NM001304509.1 | RAC3/NM005052.2 |
| ALMS1/NM015120.4 | HDHD3/NM031219.3 | RACGAP1/NM001126104.2 |
| ALOX12/NM000697.2 | HDLBP/NM001320965.1 | RACK1/NM006098.4 |
| ALOX12B/NM001139.2 | HDLBP/NM001320966.1 | RAD1/NM002853.3 |
| ALOX15/NM001140.3 | HDLBP/NM005336.5 | RAD17/NM133339.2 |
| ALOX15B/NM001141.2 | HDLBP/NM203346.4 | RAD18/NM020165.3 |
| ALOX5/NM000698.4 | HEATR1/NM018072.5 | RAD21/NM006265.2 |
| ALOX5AP/NM001629.3 | HEATR3/NM182922.2 | RAD21L1/NM001136566.2 |
| ALOXE3/NM021628.2 | HEATR4/NM001220484.1 | RAD23A/NM005053.3 |
| ALPI/NM001631.4 | HEATR5A/NM015473.3 | RAD23B/NM002874.4 |
| ALPK1/NM001102406.1 | HEATR5B/NM019024.2 | RAD50/NM005732.3 |
| ALPK2/NM052947.3 | HEATR6/NM022070.4 | RAD51/NM002875.4 |
| ALPK3/NM020778.4 | HEATR9/NM152781.3 | RAD51AP1/NM006479.4 |
| ALPL/NM000478.5 | HEBP1/NM015987.4 | RAD51AP2/NM001099218.2 |
| ALPP/NM001632.4 | HEBP2/NM014320.2 | RAD51C/NM058216.2 |
| ALPPL2/NM031313.2 | HECA/NM016217.2 | RAD51D/NM002878.3 |
| ALS2/NM020919.3 | HECTD2/NM182765.4 | RAD52/NM001297419.1 |
| ALS2CL/NM001190707.1 | HECTD4/NM001109662.3 | RAD52/NM134424.3 |
| ALS2CL/NM147129.4 | HECW2/NM020760.2 | RAD54B/NM012415.3 |
| ALS2CR11/NM152525.5 | HEG1/NM020733.1 | RAD54L/NM001142548.1 |
| ALS2CR12/NM139163.3 | HELB/NM033647.4 | RAD54L2/NM001322256.1 |
| ALX1/NM006982.2 | HELQ/NM133636.3 | RAD9A/NM004584.2 |
| ALX3/NM006492.2 | HELT/NM001300781.1 | RAD9B/NM152442.3 |
| ALX4/NM021926.3 | HELZ/NM014877.3 | RADIL/NM018059.4 |
| ALYREF/NM005782.3 | HEMGN/NM018437.4 | RAE1/NM001015885.1 |
| AMACR/NM014324.5 | HEMGN/NM197978.2 | RAE1/NM003610.3 |
| AMBN/NM016519.5 | HEMK1/NM016173.4 | RAET1E/NM139165.2 |
| AMBRA1/NM017749.3 | HENMT1/NM001102592.1 | RAF1/NM002880.3 |
| AMD1/NM001634.5 | HENMT1/NM144584.2 | RAG1/NM000448.2 |
| AMDHD1/NM152435.2 | HEPACAM/NM152722.4 | RAG2/NM001243786.1 |
| AMER2/NM152704.3 | HEPACAM2/NM001039372.2 | RAI1/NM030665.3 |
| AMFR/NM001144.5 | HEPHL1/NM001098672.1 | RAI14/NM001145520.1 |
| AMH/NM000479.3 | HEPN1/NM001037558.2 | RAI14/NM001145521.1 |
| AMHR2/NM020547.2 | HERC1/NM003922.3 | RAI14/NM015577.2 |
| AMIGO1/NM020703.2 | HERC2/NM004667.5 | RALA/NM005402.3 |
| AMIGO2/NM181847.4 | HERC3/NM014606.2 | RALB/NM002881.2 |
| AMIGO3/NM198722.2 | HERC4/NM015601.3 | RALBP1/NM006788.3 |
| AMMECR1L/NM001199140.1 | HERC5/NM016323.3 | RALGAPA1/NM194301.2 |
| AMMECR1L/NM031445.2 | HERC6/NM017912.3 | RALGAPA2/NM020343.3 |
| AMN/NM030943.3 | HERPUD1/NM014685.3 | RALGAPB/NM001282917.1 |
| AMN1/NM001113402.1 | HERPUD2/NM022373.4 | RALGDS/NM006266.3 |
| AMOTL1/NM130847.2 | HES1/NM005524.3 | RALGPS1/NM001322321.1 |
| AMOTL2/NM016201.3 | HES2/NM019089.4 | RALGPS2/NM152663.4 |
| AMPD2/NM139156.3 | HES3/NM001024598.3 | RALY/NM016732.2 |
| AMPD3/NM000480.2 | HES4/NM021170.3 | RALYL/NM001100393.1 |
| AMPH/NM001635.3 | HES5/NM001010926.3 | RALYL/NM173848.5 |
| AMT/NM000481.3 | HES6/NM018645.5 | RAMP1/NM005855.3 |
| AMY1A/NM001008221.1 | HES7/NM032580.3 | RAMP2/NM005854.2 |
| AMY1A/NM004038.3 | HESX1/NM003865.2 | RAMP3/NM005856.2 |
| AMY1B/NM001008218.1 | HEXA/NM000520.5 | RAN/NM006325.4 |
| AMY1C/NM001008219.1 | HEXB/NM000521.3 | RANBP10/NM020850.2 |
| AMY2A/NM000699.3 | HEXDC/NM173620.2 | RANBP17/NM022897.4 |
| AMY2B/NM020978.4 | HEXIM1/NM006460.2 | RANBP2/NM006267.4 |
| AMZ1/NM133463.3 | HEXIM2/NM001303440.1 | RANBP3/NM007322.2 |
| AMZ2/NM001033572.1 | HEXIM2/NM001303442.1 | RANBP3L/NM145000.4 |
| ANAPC1/NM022662.3 | HEXIM2/NM144608.2 | RANBP6/NM012416.3 |
| ANAPC10/NM001256707.1 | HEY1/NM012258.3 | RANBP9/NM005493.2 |
| ANAPC10/NM001256708.1 | HEY2/NM012259.2 | RANGAP1/NM001278651.1 |
| ANAPC11/NM001002245.2 | HEYL/NM014571.3 | RANGAP1/NM001317930.1 |
| ANAPC11/NM001289414.1 | HFE/NM000410.3 | RANGAP1/NM002883.3 |
| ANAPC11/NM001289416.1 | HFE2/NM213653.3 | RAP1A/NM001291896.1 |
| ANAPC13/NM001242374.1 | HFM1/NM001017975.4 | RAP1A/NM002884.3 |
| ANAPC13/NM001242375.1 | HGC6.3/NM001129895.2 | RAP1B/NM001010942.2 |
| ANAPC13/NM015391.3 | HGD/NM000187.3 | RAP1GAP/NM002885.2 |
| ANAPC15/NM001278491.1 | HGF/NM000601.5 | RAP1GDS1/NM001100427.1 |
| ANAPC15/NM001278492.1 | HGFAC/NM001528.3 | RAP2A/NM021033.6 |
| ANAPC15/NM001278494.1 | HGH1/NM016458.3 | RAP2B/NM002886.3 |
| ANAPC15/NM014042.2 | HGS/NM004712.4 | RAPGEF1/NM198679.1 |
| ANAPC16/NM001242547.1 | HGSNAT/NM152419.2 | RAPGEF3/NM001098532.2 |
| ANAPC2/NM013366.3 | HHAT/NM001122834.3 | RAPGEF3/NM006105.5 |
| ANAPC4/NM013367.2 | HHAT/NM001170580.2 | RAPGEF4/NM007023.3 |
| ANAPC5/NM016237.4 | HHAT/NM018194.5 | RAPGEF5/NM012294.3 |
| ANAPC7/NM016238.2 | HHEX/NM002729.4 | RAPGEF6/NM016340.5 |
| ANG/NM001097577.2 | HHIP/NM022475.2 | RAPH1/NM213589.2 |
| ANG/NM001145.4 | HHIPL1/NM001127258.1 | RAPSN/NM005055.4 |
| ANGEL1/NM015305.3 | HHIPL2/NM024746.3 | RARA/NM000964.3 |
| ANGEL2/NM144567.4 | HHLA1/NM001145095.1 | RARA/NM001145301.2 |
| ANGPT1/NM001146.4 | HHLA2/NM001282556.1 | RARB/NM000965.4 |
| ANGPT2/NM001147.2 | HHLA2/NM001282557.1 | RARG/NM000966.5 |
| ANGPT4/NM015985.3 | HHLA2/NM007072.3 | RARRES1/NM206963.1 |
| ANGPTL1/NM004673.3 | HIBADH/NM152740.3 | RARRES2/NM002889.3 |
| ANGPTL2/NM012098.2 | HIBCH/NM014362.3 | RARRES3/NM004585.4 |
| ANGPTL3/NM014495.3 | HIC1/NM001098202.1 | RARS/NM002887.3 |
| ANGPTL4/NM139314.2 | HIC2/NM015094.2 | RARS2/NM020320.4 |
| ANGPTL5/NM178127.4 | HID1/NM030630.2 | RASA1/NM002890.2 |
| ANGPTL6/NM031917.2 | HIF1A/NM001530.3 | RASA3/NM007368.3 |
| ANGPTL7/NM021146.3 | HIF1AN/NM017902.2 | RASA4/NM006989.5 |
| ANGPTL8/NM018687.6 | HIF3A/NM152795.3 | RASAL1/NM004658.2 |
| ANHX/NM001191054.1 | HIGD1A/NM001099669.1 | RASAL2/NM170692.2 |
| ANK1/NM020476.2 | HIGD1B/NM001271880.1 | RASAL3/NM022904.1 |
| ANK2/NM001148.4 | HIGD1B/NM016438.3 | RASD1/NM016084.4 |
| ANK3/NM020987.4 | HIGD1C/NM001109619.1 | RASD2/NM014310.3 |
| ANKAR/NM144708.3 | HIGD2A/NM138820.2 | RASEF/NM152573.3 |
| ANKDD1A/NM182703.5 | HILPDA/NM001098786.1 | RASGEF1A/NM145313.3 |
| ANKDD1B/NM001276713.1 | HINFP/NM198971.2 | RASGEF1B/NM152545.2 |
| ANKEF1/NM198798.2 | HINT1/NM005340.6 | RASGEF1C/NM175062.3 |
| ANKFN1/NM153228.2 | HINT2/NM032593.2 | RASGRF1/NM001145648.1 |
| ANKFY1/NM016376.3 | HINT3/NM138571.4 | RASGRF2/NM006909.2 |
| ANKH/NM054027.4 | HIP1/NM005338.6 | RASGRP1/NM005739.3 |
| ANKHD1/NM017747.2 | HIP1R/NM003959.2 | RASGRP2/NM001098670.1 |
| ANKIB1/NM019004.1 | HIPK1/NM198268.2 | RASGRP2/NM001098671.1 |
| ANKKI/NM178510.1 | HIPK2/NM022740.4 | RASGRP2/NM153819.1 |
| ANKLE1/NM152363.5 | HIPK3/NM005734.4 | RASGRP3/NM001139488.1 |
| ANKLE2/NM015114.2 | HIPK4/NM144685.4 | RASGRP3/NM170672.2 |
| ANKMY1/NM016552.3 | HIRA/NM003325.3 | RASGRP4/NM170604.2 |
| ANKMY2/NM020319.2 | HIRIP3/NM003609.4 | RASIP1/NM017805.2 |
| ANKRA2/NM023039.4 | HIST1H1A/NM005325.3 | RASL10A/NM006477.4 |
| ANKRD1/NM014391.2 | HIST1H1B/NM005322.2 | RASL10B/NM033315.3 |
| ANKRD10/NM017664.3 | HIST1H1C/NM005319.3 | RASL11A/NM206827.1 |
| ANKRD11/NM001256182.1 | HIST1H1D/NM005320.2 | RASL11B/NM023940.2 |
| ANKRD12/NM015208.4 | HIST1H1E/NM005321.2 | RASL12/NM016563.3 |
| ANKRD13A/NM033121.1 | HIST1H1T/NM005323.3 | RASSF1/NM007182.4 |
| ANKRD13B/NM152345.4 | HIST1H2AA/NM170745.3 | RASSF10/NM001080521.2 |
| ANKRD13D/NM207354.2 | HIST1H2AB/NM003513.2 | RASSF2/NM014737.2 |
| ANKRD16/NM019046.2 | HIST1H2AC/NM003512.3 | RASSF2/NM170774.1 |
| ANKRD17/NM032217.4 | HIST1H2AE/NM021052.2 | RASSF3/NM178169.3 |
| ANKRD18A/NM147195.2 | HIST1H2AG/NM021064.4 | RASSF4/NM032023.3 |
| ANKRD18B/NM001244752.1 | HIST1H2AH/NM080596.2 | RASSF5/NM182663.3 |
| ANKRD20A1/NM032250.3 | HIST1H2AI/NM003509.2 | RASSF6/NM177532.4 |
| ANKRD20A2/NM001012421.1 | HIST1H2AJ/NM021066.2 | RASSF7/NM003475.3 |
| ANKRD20A3/NM001012419.2 | HIST1H2AK/NM003510.2 | RASSF8/NM001164746.1 |
| ANKRD20A4/NM001098805.1 | HIST1H2AL/NM003511.2 | RASSF8/NM001164747.1 |
| ANKRD22/NM144590.2 | HIST1H2AM/NM003514.2 | RASSF8/NM001164748.1 |
| ANKRD23/NM144994.7 | HIST1H2BA/NM170610.2 | RASSF9/NM005447.3 |
| ANKRD26/NM014915.2 | HIST1H2BB/NM021062.2 | RAVER1/NM133452.2 |
| ANKRD28/NM015199.3 | HIST1H2BC/NM003526.2 | RAVER2/NM018211.3 |
| ANKRD29/NM173505.3 | HIST1H2BD/NM021063.3 | RAX/NM013435.2 |
| ANKRD30A/NM052997.2 | HIST1H2BE/NM003523.2 | RAX2/NM032753.3 |
| ANKRD31/NM001164443.1 | HIST1H2BF/NM003522.3 | RB1/NM000321.2 |
| ANKRD33B/NM001164440.1 | HIST1H2BG/NM003518.3 | RB1CC1/NM014781.4 |
| ANKRD34A/NM001039888.3 | HIST1H2BH/NM003524.2 | RBAK/NM001204456.1 |
| ANKRD34B/NM001004441.2 | HIST1H2BI/NM003525.2 | RBAK/NM021163.3 |
| ANKRD34C/NM001146341.1 | HIST1H2BJ/NM021058.3 | RBBP4/NM005610.2 |
| ANKRD35/NM144698.4 | HIST1H2BK/NM080593.2 | RBBP6/NM006910.4 |
| ANKRD36/NM001164315.1 | HIST1H2BL/NM003519.3 | RBBP8/NM002894.2 |
| ANKRD36B/NM025190.3 | HIST1H2BM/NM003521.2 | RBBP8/NM203291.1 |
| ANKRD36C/NM001310154.1 | HIST1H2BN/NM003520.3 | RBBP8NL/NM080833.2 |
| ANKRD37/NM181726.3 | HIST1H2BO/NM003527.4 | RBBP9/NM006606.2 |
| ANKRD39/NM016466.5 | HIST1H3A/NM003529.2 | RBCK1/NM031229.3 |
| ANKRD40/NM052855.3 | HIST1H3B/NM003537.3 | RBFA/NM024805.2 |
| ANKRD42/NM001300975.1 | HIST1H3C/NM003531.2 | RBFOX1/NM145891.2 |
| ANKRD44/NM001195144.1 | HIST1H3D/NM003530.4 | RBFOX2/NM001082578.1 |
| ANKRD45/NM198493.2 | HIST1H3E/NM003532.2 | RBFOX3/NM001082575.2 |
| ANKRD46/NM001270378.1 | HIST1H3F/NM021018.2 | RBKS/NM022128.2 |
| ANKRD46/NM198401.3 | HIST1H3G/NM003534.2 | RBL1/NM002895.4 |
| ANKRD49/NM017704.2 | HIST1H3H/NM003536.2 | RBL2/NM001323608.1 |
| ANKRD50/NM020337.2 | HIST1H3I/NM003533.2 | RBL2/NM005611.3 |
| ANKRD52/NM173595.3 | HIST1H3J/NM003535.2 | RBM12B/NM203390.2 |
| ANKRD53/NM024933.3 | HIST1H4A/NM003538.3 | RBM14/NM006328.3 |
| ANKRD54/NM138797.2 | HIST1H4B/NM003544.2 | RBM15/NM022768.4 |
| ANKRD55/NM024669.2 | HIST1H4C/NM003542.3 | RBM15B/NM013286.4 |
| ANKRD6/NM014942.4 | HIST1H4D/NM003539.3 | RBM17/NM001145547.1 |
| ANKRD60/NM001304369.1 | HIST1H4E/NM003545.3 | RBM17/NM032905.4 |
| ANKRD61/NM001271700.1 | HIST1H4F/NM003540.3 | RBM18/NM033117.3 |
| ANKRD63/NM001190479.2 | HIST1H4G/NM003547.2 | RBM19/NM001146699.1 |
| ANKRD65/NM001145210.2 | HIST1H4I/NM003495.2 | RBM20/NM001134363.2 |
| ANKRD66/NM001162435.2 | HIST1H4J/NM021968.3 | RBM23/NM001077351.1 |
| ANKRD7/NM019644.3 | HIST1H4K/NM003541.2 | RBM24/NM001143942.1 |
| ANKRD9/NM152326.2 | HIST1H4L/NM003546.2 | RBM25/NM021239.2 |
| ANKS1A/NM015245.2 | HIST2H2AA3/NM003516.2 | RBM26/NM022118.4 |
| ANKS3/NM133450.3 | HIST2H2AA4/NM001040874.1 | RBM27/NM018989.1 |
| ANKS4B/NM145865.2 | HIST2H2AB/NM175065.2 | RBM33/NM053043.2 |
| ANKS6/NM173551.4 | HIST2H2AC/NM003517.2 | RBM34/NM015014.2 |
| ANKUB1/NM001144960.2 | HIST2H2BE/NM003528.2 | RBM38/NM017495.5 |
| ANKZF1/NM001042410.1 | HIST2H2BF/NM001024599.4 | RBM4/NM002896.3 |
| ANKZF1/NM018089.2 | HIST2H3A/NM001005464.2 | RBM43/NM198557.2 |
| ANLN/NM018685.4 | HIST2H3C/NM021059.2 | RBM44/NM001080504.2 |
| ANO1/NM018043.5 | HIST2H3D/NM001123375.2 | RBM47/NM019027.3 |
| ANO10/NM018075.3 | HIST2H4A/NM003548.2 | RBM48/NM032120.3 |
| ANO2/NM001278597.1 | HIST2H4B/NM001034077.4 | RBM4B/NM031492.3 |
| ANO3/NM031418.3 | HIST3H2A/NM033445.2 | RBM5/NM005778.3 |
| ANO4/NM178826.3 | HIST3H2BB/NM175055.2 | RBM6/NM005777.2 |
| ANO5/NM213599.2 | HIST3H3/NM003493.2 | RBM7/NM016090.3 |
| ANO6/NM001025356.2 | HIST4H4/NM175054.2 | RBM8A/NM005105.4 |
| ANO7/NM001001891.3 | HIVEP1/NM002114.3 | RBMS1/NM002897.4 |
| ANO8/NM020959.2 | HIVEP2/NM006734.3 | RBMS2/NM002898.3 |
| ANP32A/NM006305.3 | HIVEP3/NM024503.4 | RBMS3/NM001003793.2 |
| ANP32B/NM006401.2 | HJURP/NM018410.4 | RBMXL2/NM014469.4 |
| ANP32C/NM012403.1 | HK1/NM000188.2 | RBP1/NM002899.3 |
| ANP32D/NM012404.2 | HK2/NM000189.4 | RBP2/NM004164.2 |
| ANP32E/NM030920.4 | HK3/NM002115.2 | RBP3/NM002900.2 |
| ANPEP/NM001150.2 | HKDC1/NM025130.3 | RBP4/NM001323517.1 |
| ANTXR1/NM032208.2 | HKR1/NM181786.2 | RBP4/NM006744.3 |
| ANTXR2/NM058172.5 | HLA-A/NM002116.7 | RBP7/NM052960.2 |
| ANTXRL/NM001278688.2 | HLA-C/NM002117.5 | RBPMS/NM001008710.2 |
| ANXA1/NM000700.2 | HLA-DMA/NM006120.3 | RBPMS2/NM194272.1 |
| ANXA10/NM007193.4 | HLA-DMB/NM002118.4 | RBSN/NM022340.3 |
| ANXA11/NM001278407.1 | HLA-DOA/NM002119.3 | RBX1/NM014248.3 |
| ANXA11/NM001278408.1 | HLA-DPA1/NM001242524.1 | RC3H1/NM172071.3 |
| ANXA11/NM145869.1 | HLA-DPA1/NM033554.3 | RC3H2/NM001100588.1 |
| ANXA13/NM001003954.2 | HLA-DPB1/NM002121.5 | RCAN2/NM005822.3 |
| ANXA3/NM005139.2 | HLA-DQA1/NM002122.3 | RCAN3/NM001251978.1 |
| ANXA4/NM001153.4 | HLA-DQA2/NM020056.4 | RCBTB1/NM018191.3 |
| ANXA4/NM001320698.1 | HLA-DQB1/NM002123.4 | RCBTB2/NM001268.3 |
| ANXA6/NM001155.4 | HLA-DQB2/NM001198858.1 | RCC1/NM001048199.2 |
| ANXA7/NM004034.3 | HLA-DRA/NM019111.4 | RCC1/NM001269.4 |
| ANXA8/NM001040084.2 | HLA-DRB1/NM002124.3 | RCC1L/NM030798.4 |
| ANXA8L1/NM001098845.2 | HLA-DRB5/NM002125.3 | RCC2/NM001136204.2 |
| ANXA9/NM003568.2 | HLA-E/NM005516.5 | RCC2/NM018715.3 |
| AOAH/NM001637.3 | HLA-F/NM001098479.1 | RCCD1/NM001017919.1 |
| AOC1/NM001091.3 | HLA-G/NM002127.5 | RCE1/NM005133.2 |
| AOC2/NM009590.2 | HLCS/NM000411.6 | RCHY1/NM015436.3 |
| AOC3/NM003734.3 | HLCS/NM001242784.1 | RCL1/NM005772.4 |
| AOX1/NM001159.3 | HLCS/NM001242785.1 | RCN1/NM002901.2 |
| AP1B1/NM001127.3 | HLF/NM002126.4 | RCN2/NM002902.2 |
| AP1G1/NM001128.5 | HLTF/NM003071.3 | RCN3/NM020650.2 |
| AP1G2/NM003917.4 | HLX/NM021958.3 | RCOR1/NM015156.3 |
| AP1M1/NM032493.3 | HM13/NM030789.3 | RCOR2/NM173587.3 |
| AP1M2/NM005498.4 | HMBOX1/NM001135726.2 | RCOR3/NM001136223.1 |
| AP1S1/NM001283.3 | HMBOX1/NM024567.4 | RCSD1/NM052862.3 |
| AP1S3/NM001039569.1 | HMBS/NM000190.3 | RCVRN/NM002903.2 |
| AP2A1/NM014203.2 | HMCES/NM001006109.1 | RD3/NM183059.2 |
| AP2A2/NM012305.3 | HMCES/NM020187.2 | RD3L/NM001257268.1 |
| AP2B1/NM001030006.1 | HMCN1/NM031935.2 | RDH10/NM172037.4 |
| AP2M1/NM004068.3 | HMCN2/NM001291815.1 | RDH11/NM016026.3 |
| AP2S1/NM004069.4 | HMG20A/NM001304504.1 | RDH12/NM152443.2 |
| AP3B1/NM003664.4 | HMG20B/NM006339.2 | RDH13/NM001145971.1 |
| AP3B2/NM004644.4 | HMGA1/NM145899.2 | RDH14/NM020905.3 |
| AP3D1/NM003938.6 | HMGA1/NM145901.2 | RDH16/NM003708.4 |
| AP3M1/NM001320263.1 | HMGA2/NM003483.4 | RDH5/NM002905.3 |
| AP3M1/NM001320264.1 | HMGB1/NM001313892.1 | RDH8/NM015725.2 |
| AP3M2/NM006803.3 | HMGB1/NM001313893.1 | RDM1/NM145654.3 |
| AP3S1/NM001284.3 | HMGB1/NM002128.5 | RDX/NM002906.3 |
| AP3S2/NM005829.4 | HMGB2/NM001130688.1 | REC8/NM005132.2 |
| AP4B1/NM001253852.2 | HMGB2/NM001130689.1 | RECK/NM021111.2 |
| AP4B1/NM006594.4 | HMGB2/NM002129.3 | RECQL/NM002907.3 |
| AP4E1/NM007347.4 | HMGB4/NM145205.5 | RECQL4/NM004260.3 |
| AP4M1/NM004722.3 | HMGCL/NM000191.2 | RECQL5/NM004259.6 |
| AP4S1/NM001128126.2 | HMGCLL1/NM001042406.1 | REEP1/NM022912.2 |
| AP4S1/NM001254728.1 | HMGCR/NM000859.2 | REEP2/NM016606.3 |
| AP4S1/NM001254729.1 | HMGCS2/NM005518.3 | REEP3/NM001001330.2 |
| AP5B1/NM138368.4 | HMGN1/NM004965.6 | REEP4/NM025232.3 |
| AP5M1/NM018229.3 | HMGN2/NM005517.3 | REEP5/NM005669.4 |
| AP5S1/NM001204447.1 | HMGN3/NM004242.3 | REG1A/NM002909.4 |
| APAF1/NM181861.1 | HMGN4/NM006353.2 | REG1B/NM006507.3 |
| APBA1/NM001163.3 | HMGXB3/NM014983.2 | REG3A/NM002580.2 |
| APBA2/NM005503.3 | HMGXB4/NM001003681.2 | REG3A/NM138938.2 |
| APBA3/NM004886.3 | HMHA1/NM012292.4 | REG3G/NM198448.3 |
| APBB1/NM145689.2 | HMHB1/NM021182.1 | REG4/NM001159352.1 |
| APBB1IP/NM019043.3 | HMMR/NM012484.2 | REL/NM002908.3 |
| APBB2/NM173075.4 | HMOX1/NM002133.2 | RELA/NM021975.3 |
| APBB3/NM133173.2 | HMOX2/NM001127205.1 | RELB/NM006509.3 |
| APC2/NM005883.2 | HMOX2/NM001127206.2 | RELL1/NM001085399.1 |
| APCDD1/NM153000.4 | HMOX2/NM001286270.1 | RELL2/NM001130029.1 |
| APCDD1L/NM153360.2 | HMOX2/NM002134.3 | RELN/NM173054.2 |
| APCS/NM001639.3 | HMP19/NM015980.4 | RELT/NM032871.3 |
| APEH/NM001640.3 | HMX1/NM018942.2 | RELT/NM152222.1 |
| APELA/NM001297550.1 | HMX2/NM005519.1 | REM1/NM014012.5 |
| APEX1/NM080648.2 | HMX3/NM001105574.1 | REM2/NM173527.2 |
| APH1A/NM001077628.2 | HN1/NM016185.3 | REN/NM000537.3 |
| API5/NM006595.3 | HN1L/NM144570.2 | REP15/NM001029874.1 |
| APIP/NM015957.3 | HNF1A/NM000545.6 | REPIN1/NM014374.3 |
| APITD1/NM199294.2 | HNF1B/NM000458.3 | REPS1/NM031922.4 |
| APLF/NM173545.2 | HNF4A/NM000457.4 | RER1/NM007033.4 |
| APLNR/NM005161.4 | HNMT/NM006895.2 | RERE/NM012102.3 |
| APLP1/NM001024807.2 | HNRNPA0/NM006805.3 | RERG/NM032918.2 |
| APLP2/NM001642.2 | HNRNPA1/NM002136.3 | RERGL/NM001286201.1 |
| APMAP/NM020531.2 | HNRNPA1L2/NM001011725.1 | RESP18/NM001007089.3 |
| APOA1/NM000039.2 | HNRNPA2B1/NM002137.3 | REST/NM001193508.1 |
| APOA1/NM001318017.1 | HNRNPA3/NM194247.2 | REST/NM005612.4 |
| APOA1/NM001318018.1 | HNRNPAB/NM031266.2 | RET/NM020975.4 |
| APOA2/NM001643.1 | HNRNPC/NM031314.2 | RETN/NM001193374.1 |
| APOA4/NM000482.3 | HNRNPCL1/NM001013631.2 | RETNLB/NM032579.2 |
| APOB/NM000384.2 | HNRNPCL2/NM001136561.3 | RETSAT/NM017750.3 |
| APOBEC1/NM001304566.1 | HNRNPCL3/NM001146181.2 | REV1/NM016316.3 |
| APOBEC1/NM001644.4 | HNRNPCL4/NM001302551.1 | REV3L/NM002912.4 |
| APOBEC2/NM006789.3 | HNRNPD/NM031370.2 | REXO1/NM020695.3 |
| APOBEC3A/NM145699.3 | HNRNPDL/NM031372.3 | REXO2/NM015523.3 |
| APOBEC3B/NM004900.4 | HNRNPF/NM001098204.1 | REXO4/NM020385.3 |
| APOBEC3C/NM014508.2 | HNRNPF/NM001098205.1 | RFC1/NM002913.4 |
| APOBEC3D/NM152426.3 | HNRNPF/NM001098206.1 | RFC2/NM181471.2 |
| APOBEC3F/NM145298.5 | HNRNPF/NM001098207.1 | RFC3/NM002915.3 |
| APOBEC3G/NM021822.3 | HNRNPF/NM001098208.1 | RFC4/NM002916.3 |
| APOBEC3H/NM181773.4 | HNRNPF/NM004966.3 | RFC4/NM181573.2 |
| APOBEC4/NM203454.2 | HNRNPH1/NM001257293.1 | RFC5/NM007370.5 |
| APOBR/NM018690.3 | HNRNPH1/NM005520.2 | RFESD/NM173362.3 |
| APOC1/NM001321065.1 | HNRNPH3/NM012207.2 | RFFL/NM001017368.1 |
| APOC1/NM001645.4 | HNRNPK/NM002140.4 | RFK/NM018339.5 |
| APOC2/NM000483.4 | HNRNPK/NM031263.3 | RFNG/NM002917.1 |
| APOC4/NM001646.2 | HNRNPL/NM001533.2 | RFPL1/NM021026.2 |
| APOD/NM001647.3 | HNRNPLL/NM138394.3 | RFPL2/NM001098527.2 |
| APOE/NM000041.3 | HNRNPU/NM004501.3 | RFPL3/NM006604.2 |
| APOE/NM001302689.1 | HNRNPUL1/NM007040.5 | RFPL4A/NM001145014.1 |
| APOE/NM001302690.1 | HNRNPUL2/NM001079559.2 | RFPL4AL1/NM001277397.1 |
| APOF/NM001638.2 | HOGA1/NM138413.3 | RFPL4B/NM001013734.2 |
| APOH/NM000042.2 | HOMER1/NM004272.4 | RFT1/NM052859.3 |
| APOL1/NM145343.2 | HOMER2/NM199330.2 | RFTN1/NM015150.1 |
| APOL2/NM030882.3 | HOMER3/NM001145722.1 | RFTN2/NM144629.2 |
| APOL2/NM145637.2 | HOMER3/NM004838.3 | RFWD2/NM022457.6 |
| APOL3/NM145640.2 | HOMEZ/NM020834.2 | RFWD3/NM018124.3 |
| APOL4/NM145660.2 | HOOK2/NM013312.2 | RFX1/NM002918.4 |
| APOL5/NM030642.1 | HOOK3/NM032410.3 | RFX2/NM000635.3 |
| APOL6/NM030641.3 | HOPX/NM001145459.1 | RFX3/NM134428.2 |
| APOLD1/NM030817.2 | HOPX/NM139212.3 | RFX4/NM213594.2 |
| APOM/NM019101.2 | HORMAD1/NM032132.4 | RFX5/NM000449.3 |
| APP/NM000484.3 | HORMAD2/NM152510.2 | RFX7/NM022841.5 |
| APPBP2/NM006380.3 | HOTS/NM001293171.2 | RFXANK/NM003721.3 |
| APPL1/NM012096.2 | HOXA1/NM005522.4 | RFXAP/NM000538.3 |
| APPL2/NM018171.3 | HOXA10/NM018951.3 | RGCC/NM014059.2 |
| APRT/NM000485.2 | HOXA11/NM005523.5 | RGL1/NM015149.4 |
| APTX/NM001195249.1 | HOXA13/NM000522.4 | RGL2/NM004761.4 |
| APTX/NM175073.2 | HOXA2/NM006735.3 | RGL3/NM001035223.3 |
| AQP1/NM198098.2 | HOXA3/NM030661.4 | RGL4/NM153615.1 |
| AQP10/NM080429.2 | HOXA3/NM153631.2 | RGMA/NM020211.2 |
| AQP11/NM173039.2 | HOXA4/NM002141.4 | RGMB/NM001012761.2 |
| AQP12A/NM198998.2 | HOXA5/NM019102.3 | RGP1/NM001080496.2 |
| AQP12B/NM001102467.1 | HOXA6/NM024014.3 | RGPD3/NM001144013.1 |
| AQP2/NM000486.5 | HOXA7/NM006896.3 | RGPD4/NM182588.2 |
| AQP3/NM004925.4 | HOXA9/NM152739.3 | RGPD5/NM005054.2 |
| AQP4/NM001650.5 | HOXB1/NM002144.3 | RGPD6/NM001123363.3 |
| AQP5/NM001651.3 | HOXB13/NM006361.5 | RGPD8/NM001164463.1 |
| AQP6/NM001652.3 | HOXB2/NM002145.3 | RGS1/NM002922.3 |
| AQP7/NM001170.2 | HOXB3/NM002146.4 | RGS10/NM001005339.1 |
| AQP8/NM001169.2 | HOXB4/NM024015.4 | RGS11/NM003834.2 |
| AQP9/NM020980.4 | HOXB5/NM002147.3 | RGS12/NM198229.2 |
| AQR/NM014691.2 | HOXB6/NM018952.4 | RGS13/NM144766.2 |
| ARAP1/NM015242.4 | HOXB7/NM004502.3 | RGS14/NM006480.4 |
| ARAP2/NM015230.3 | HOXB8/NM024016.3 | RGS16/NM002928.3 |
| ARAP3/NM022481.5 | HOXB9/NM024017.4 | RGS17/NM012419.4 |
| ARC/NM015193.4 | HOXC10/NM017409.3 | RGS18/NM130782.2 |
| ARCN1/NM001655.4 | HOXC11/NM014212.3 | RGS19/NM001039467.1 |
| AREG/NM001657.3 | HOXC12/NM173860.1 | RGS19/NM005873.2 |
| AREL1/NM001039479.1 | HOXC13/NM017410.2 | RGS2/NM002923.3 |
| ARF1/NM001024228.1 | HOXC4/NM014620.5 | RGS20/NM170587.3 |
| ARF1/NM001658.3 | HOXC4/NM153633.2 | RGS21/NM001039152.3 |
| ARF3/NM001659.2 | HOXC5/NM018953.3 | RGS22/NM015668.4 |
| ARF4/NM001660.3 | HOXC6/NM004503.3 | RGS3/NM144488.5 |
| ARF5/NM001662.3 | HOXC8/NM022658.3 | RGS5/NM003617.3 |
| ARF6/NM001663.3 | HOXC9/NM006897.1 | RGS6/NM004296.6 |
| ARFGAP1/NM175609.2 | HOXD1/NM024501.2 | RGS7/NM001282775.1 |
| ARFGAP2/NM032389.4 | HOXD10/NM002148.3 | RGS7BP/NM001029875.2 |
| ARFGAP3/NM014570.4 | HOXD11/NM021192.2 | RGS8/NM001102450.2 |
| ARFGEF1/NM006421.4 | HOXD12/NM021193.3 | RGS9/NM003835.3 |
| ARFGEF2/NM006420.2 | HOXD13/NM000523.3 | RGSL1/NM001137669.1 |
| ARFGEF3/NM020340.4 | HOXD3/NM006898.4 | RHAG/NM000324.2 |
| ARFIP1/NM014447.3 | HOXD4/NM014621.2 | RHBDD1/NM032276.3 |
| ARFIP2/NM012402.3 | HOXD8/NM019558.3 | RHBDD2/NM001040456.1 |
| ARFRP1/NM001267547.2 | HOXD9/NM014213.3 | RHBDD3/NM012265.1 |
| ARFRP1/NM001267548.2 | HP/NM005143.4 | RHBDF1/NM022450.3 |
| ARG1/NM000045.3 | HPCA/NM002143.2 | RHBDF2/NM024599.5 |
| ARG2/NM001172.3 | HPCAL1/NM001258357.1 | RHBDL1/NM001278720.1 |
| ARGFX/NM001012659.1 | HPCAL1/NM001258358.1 | RHBDL2/NM017821.4 |
| ARGLU1/NM018011.3 | HPCAL1/NM001258359.1 | RHBDL3/NM138328.2 |
| ARHGAP1/NM004308.3 | HPCAL1/NM002149.3 | RHCE/NM020485.4 |
| ARHGAP10/NM024605.3 | HPCAL1/NM134421.2 | RHCG/NM001321041.1 |
| ARHGAP11A/NM014783.4 | HPCAL4/NM001282396.1 | RHCG/NM016321.2 |
| ARHGAP11B/NM001039841.1 | HPCAL4/NM016257.3 | RHD/NM016124.4 |
| ARHGAP12/NM018287.6 | HPD/NM002150.2 | RHEB/NM005614.3 |
| ARHGAP15/NM018460.3 | HPDL/NM032756.2 | RHEBL1/NM144593.2 |
| ARHGAP17/NM001006634.2 | HPF1/NM017867.2 | RHNO1/NM001252499.2 |
| ARHGAP18/NM033515.2 | HPGD/NM000860.5 | RHO/NM000539.3 |
| ARHGAP19/NM032900.5 | HPGDS/NM014485.2 | RHOA/NM001313941.1 |
| ARHGAP20/NM020809.3 | HPN/NM002151.2 | RHOB/NM004040.3 |
| ARHGAP21/NM020824.3 | HPR/NM020995.3 | RHOBTB1/NM001242359.1 |
| ARHGAP22/NM021226.3 | HPS1/NM000195.4 | RHOBTB2/NM015178.2 |
| ARHGAP23/NM001199417.1 | HPS3/NM032383.4 | RHOBTB3/NM014899.3 |
| ARHGAP24/NM001025616.2 | HPS4/NM022081.5 | RHOC/NM001042679.1 |
| ARHGAP25/NM001007231.2 | HPS5/NM181507.1 | RHOC/NM175744.4 |
| ARHGAP26/NM015071.4 | HPS6/NM024747.5 | RHOD/NM014578.3 |
| ARHGAP27/NM001282290.1 | HPSE/NM001098540.2 | RHOF/NM019034.2 |
| ARHGAP29/NM004815.3 | HPSE/NM006665.5 | RHOG/NM001665.3 |
| ARHGAP30/NM181720.2 | HPSE2/NM021828.4 | RHOH/NM001278367.1 |
| ARHGAP31/NM020754.3 | HPX/NM000613.2 | RHOH/NM001278368.1 |
| ARHGAP32/NM001142685.1 | HR/NM005144.4 | RHOH/NM001278369.1 |
| ARHGAP33/NM052948.3 | HRAS/NM001130442.2 | RHOH/NM004310.4 |
| ARHGAP35/NM004491.4 | HRASLS2/NM017878.1 | RHOJ/NM020663.4 |
| ARHGAP39/NM001308207.1 | HRASLS5/NM054108.3 | RHOQ/NM012249.3 |
| ARHGAP39/NM001308208.1 | HRC/NM002152.2 | RHOT1/NM001033568.2 |
| ARHGAP42/NM152432.2 | HRCT1/NM001039792.1 | RHOT2/NM138769.2 |
| ARHGAP44/NM014859.5 | HRH1/NM000861.3 | RHOU/NM021205.5 |
| ARHGAP5/NM001030055.1 | HRH1/NM001098211.1 | RHOV/NM133639.3 |
| ARHGAP8/NM181335.2 | HRH1/NM001098212.1 | RHPN1/NM052924.2 |
| ARHGAP9/NM032496.3 | HRH1/NM001098213.1 | RHPN2/NM033103.4 |
| ARHGDIB/NM001321420.1 | HRH2/NM022304.2 | RIBC2/NM015653.4 |
| ARHGDIB/NM001321421.1 | HRH3/NM007232.2 | RIC1/NM020829.3 |
| ARHGDIB/NM001321422.1 | HRH4/NM021624.3 | RIC3/NM024557.4 |
| ARHGDIB/NM001321423.1 | HRK/NM003806.2 | RIC8A/NM001286134.1 |
| ARHGDIG/NM001176.3 | HRNR/NM001009931.2 | RICTOR/NM152756.4 |
| ARHGEF1/NM199002.1 | HS1BP3/NM022460.3 | RIDA/NM005836.2 |
| ARHGEF10/NM014629.3 | HS2ST1/NM012262.3 | RIF1/NM018151.4 |
| ARHGEF10L/NM018125.3 | HS3ST1/NM005114.2 | RILP/NM031430.2 |
| ARHGEF11/NM198236.2 | HS3ST2/NM006043.1 | RILPL1/NM178314.4 |
| ARHGEF12/NM015313.2 | HS3ST3A1/NM006042.2 | RILPL2/NM145058.2 |
| ARHGEF15/NM025014.1 | HS3ST3B1/NM006041.2 | RIMBP2/NM015347.4 |
| ARHGEF15/NM173728.3 | HS3ST4/NM006040.2 | RIMBP3/NM015672.1 |
| ARHGEF16/NM014448.3 | HS3ST5/NM153612.3 | RIMBP3B/NM001128635.1 |
| ARHGEF17/NM014786.3 | HS3ST6/NM001009606.3 | RIMBP3C/NM001128633.1 |
| ARHGEF18/NM015318.3 | HS6ST1/NM004807.2 | RIMKLA/NM173642.3 |
| ARHGEF19/NM153213.3 | HS6ST3/NM153456.3 | RIMKLB/NM001297776.1 |
| ARHGEF2/NM004723.3 | HSBP1/NM001537.3 | RIMKLB/NM020734.3 |
| ARHGEF25/NM182947.3 | HSBP1L1/NM001136180.1 | RIMS2/NM001100117.2 |
| ARHGEF26/NM001251962.1 | HSCB/NM172002.4 | RIMS3/NM014747.2 |
| ARHGEF26/NM015595.3 | HSD11B1/NM005525.3 | RIMS4/NM182970.3 |
| ARHGEF28/NM001080479.2 | HSD11B1/NM181755.2 | RIN1/NM004292.2 |
| ARHGEF3/NM019555.2 | HSD11B1L/NM198706.2 | RIN2/NM001242581.1 |
| ARHGEF33/NM001145451.2 | HSD11B2/NM000196.3 | RIN3/NM024832.4 |
| ARHGEF35/NM001003702.2 | HSD17B1/NM000413.2 | RING1/NM002931.3 |
| ARHGEF37/NM001001669.2 | HSD17B11/NM016245.4 | RINL/NM198445.3 |
| ARHGEF38/NM001242729.1 | HSD17B12/NM016142.2 | RINT1/NM021930.4 |
| ARHGEF39/NM032818.2 | HSD17B13/NM178135.4 | RIOK1/NM031480.2 |
| ARHGEF4/NM015320.3 | HSD17B14/NM016246.2 | RIOK2/NM018343.2 |
| ARHGEF40/NM018071.4 | HSD17B2/NM002153.2 | RIOK3/NM003831.3 |
| ARHGEF5/NM005435.3 | HSD17B3/NM000197.1 | RIPK1/NM003804.4 |
| ARHGEF7/NM001320852.1 | HSD17B6/NM003725.3 | RIPK2/NM003821.5 |
| ARID1A/NM006015.4 | HSD17B7/NM016371.3 | RIPK3/NM006871.3 |
| ARID1B/NM017519.2 | HSD17B8/NM014234.4 | RIPK4/NM020639.2 |
| ARID2/NM152641.2 | HSD3B7/NM025193.3 | RIPPLY3/NM018962.2 |
| ARID3A/NM005224.2 | HSDL1/NM031463.4 | RIT1/NM006912.5 |
| ARID3B/NM006465.3 | HSDL2/NM032303.4 | RITA1/NM032848.2 |
| ARID3C/NM001017363.1 | HSF1/NM005526.2 | RLBP1/NM000326.4 |
| ARID4A/NM002892.3 | HSF2/NM004506.3 | RLF/NM012421.3 |
| ARID5A/NM212481.2 | HSF2BP/NM007031.1 | RLN1/NM006911.3 |
| ARID5B/NM032199.2 | HSF4/NM001040667.2 | RLN2/NM134441.2 |
| ARIH1/NM005744.3 | HSF5/NM001080439.2 | RLN3/NM080864.3 |
| ARIH2/NM006321.3 | HSH2D/NM032855.3 | RMDN1/NM016033.2 |
| ARIH2OS/NM001123040.1 | HSP90AA1/NM005348.3 | RMDN2/NM001170791.2 |
| ARL1/NM001177.5 | HSP90AB1/NM001271970.1 | RMDN2/NM001170792.2 |
| ARL10/NM173664.5 | HSP90AB1/NM007355.3 | RMDN3/NM018145.2 |
| ARL11/NM138450.5 | HSP90B1/NM003299.2 | RMI1/NM024945.2 |
| ARL13B/NM182896.2 | HSPA12A/NM025015.2 | RMI2/NM152308.2 |
| ARL14/NM025047.2 | HSPA12B/NM052970.4 | RMND1/NM001271937.1 |
| ARL14EP/NM152316.2 | HSPA13/NM006948.4 | RMND5A/NM022780.3 |
| ARL14EPL/NM001195581.1 | HSPA14/NM016299.3 | RMND5B/NM022762.4 |
| ARL15/NM019087.2 | HSPA1A/NM005345.5 | RNASE1/NM198232.2 |
| ARL16/NM001040025.1 | HSPA1B/NM005346.4 | RNASE1/NM198235.2 |
| ARL17A/NM001113738.1 | HSPA1L/NM005527.3 | RNASE10/NM001012975.1 |
| ARL17B/NM001039083.3 | HSPA2/NM021979.3 | RNASE11/NM145250.3 |
| ARL2/NM001667.3 | HSPA4/NM002154.3 | RNASE12/NM001024822.2 |
| ARL2BP/NM012106.3 | HSPA4L/NM014278.3 | RNASE13/NM001012264.3 |
| ARL3/NM004311.3 | HSPA5/NM005347.4 | RNASE2/NM002934.2 |
| ARL4A/NM001195396.1 | HSPA6/NM002155.4 | RNASE3/NM002935.2 |
| ARL4A/NM212460.3 | HSPA8/NM006597.5 | RNASE4/NM001282193.1 |
| ARL4C/NM005737.3 | HSPA9/NM004134.6 | RNASE4/NM194431.2 |
| ARL4D/NM001661.3 | HSPB1/NM001540.3 | RNASE6/NM005615.4 |
| ARL5A/NM012097.3 | HSPB11/NM001316935.1 | RNASE8/NM138331.2 |
| ARL5B/NM178815.3 | HSPB11/NM016126.3 | RNASE9/NM001289110.1 |
| ARL5C/NM001143968.1 | HSPB2/NM001541.3 | RNASEH1/NM002936.4 |
| ARL6/NM001278293.1 | HSPB3/NM006308.2 | RNASEH2A/NM006397.2 |
| ARL6IP1/NM015161.2 | HSPB6/NM144617.2 | RNASEH2B/NM024570.3 |
| ARL6IP5/NM006407.3 | HSPB7/NM014424.4 | RNASEH2C/NM032193.3 |
| ARL6IP6/NM152522.5 | HSPB8/NM014365.2 | RNASEK/NM001004333.4 |
| ARL8A/NM138795.3 | HSPB9/NM033194.2 | RNASEL/NM021133.3 |
| ARL8B/NM018184.2 | HSPBAP1/NM024610.5 | RNASET2/NM003730.4 |
| ARMC1/NM018120.5 | HSPBP1/NM012267.4 | RND1/NM014470.3 |
| ARMC10/NM001161009.2 | HSPD1/NM002156.4 | RND2/NM005440.4 |
| ARMC12/NM145028.4 | HSPD1/NM199440.1 | RND3/NM001254738.1 |
| ARMC3/NM001282745.1 | HSPE1-MOB4/NM001202485.1 | RNF10/NM014868.4 |
| ARMC4/NM001290020.1 | HSPG2/NM005529.6 | RNF103-CHMP3/NM001198954.1 |
| ARMC4/NM018076.4 | HSPH1/NM006644.3 | RNF103/NM005667.3 |
| ARMC5/NM024742.2 | HTATIP2/NM001098521.1 | RNF11/NM014372.4 |
| ARMC6/NM033415.3 | HTATIP2/NM001098522.1 | RNF111/NM017610.7 |
| ARMC7/NM024585.3 | HTATIP2/NM006410.4 | RNF112/NM007148.4 |
| ARMC9/NM001291656.1 | HTN3/NM000200.2 | RNF113B/NM178861.4 |
| ARMS2/NM001099667.1 | HTR1A/NM000524.3 | RNF114/NM018683.3 |
| ARMT1/NM024573.2 | HTR1B/NM000863.2 | RNF115/NM014455.3 |
| ARNT/NM001668.3 | HTR1D/NM000864.4 | RNF121/NM018320.4 |
| ARNT2/NM014862.3 | HTR1E/NM000865.2 | RNF122/NM024787.3 |
| ARNTL/NM001030272.2 | HTR1F/NM000866.4 | RNF125/NM017831.3 |
| ARNTL2/NM020183.4 | HTR1F/NM001322208.1 | RNF126/NM194460.2 |
| ARPC1A/NM006409.3 | HTR1F/NM001322209.1 | RNF13/NM183381.2 |
| ARPC1B/NM005720.3 | HTR1F/NM001322210.1 | RNF130/NM018434.5 |
| ARPC2/NM005731.3 | HTR2A/NM000621.4 | RNF133/NM139175.1 |
| ARPC2/NM152862.2 | HTR2B/NM000867.4 | RNF135/NM032322.3 |
| ARPC3/NM001278556.1 | HTR3A/NM213621.3 | RNF138/NM001191324.1 |
| ARPC4-TTLL3/NM001198793.1 | HTR3B/NM006028.4 | RNF138/NM016271.4 |
| ARPC5/NM005717.3 | HTR3C/NM130770.2 | RNF139/NM007218.3 |
| ARPC5L/NM030978.2 | HTR3D/NM001163646.1 | RNF14/NM183399.2 |
| ARPP19/NM001306195.1 | HTR3E/NM182589.2 | RNF14/NM183400.2 |
| ARPP19/NM006628.5 | HTR4/NM000870.6 | RNF14/NM183401.2 |
| ARPP21/NM016300.4 | HTR5A/NM024012.3 | RNF141/NM016422.3 |
| ARRB1/NM004041.4 | HTR6/NM000871.2 | RNF144A/NM014746.4 |
| ARRB2/NM004313.3 | HTR7/NM000872.4 | RNF144B/NM182757.3 |
| ARRDC2/NM015683.1 | HTRA1/NM002775.4 | RNF145/NM144726.2 |
| ARRDC3/NM020801.2 | HTRA2/NM013247.4 | RNF149/NM173647.3 |
| ARRDC4/NM183376.2 | HTRA3/NM053044.4 | RNF150/NM020724.1 |
| ARRDC5/NM001080523.1 | HTRA4/NM153692.3 | RNF151/NM174903.4 |
| ARSA/NM000487.5 | HTT/NM002111.7 | RNF152/NM173557.2 |
| ARSB/NM000046.3 | HUNK/NM014586.1 | RNF157/NM052916.2 |
| ARSG/NM001267727.1 | HUS1/NM004507.3 | RNF165/NM152470.2 |
| ARSG/NM014960.4 | HUS1B/NM148959.3 | RNF166/NM178841.3 |
| ARSI/NM001012301.2 | HVCN1/NM001040107.1 | RNF167/NM015528.2 |
| ARSJ/NM024590.3 | HVCN1/NM032369.3 | RNF169/NM001098638.1 |
| ARSK/NM198150.2 | HYAL1/NM033159.3 | RNF170/NM001160223.1 |
| ART1/NM004314.2 | HYAL1/NM153281.1 | RNF175/NM173662.2 |
| ART3/NM001179.5 | HYAL2/NM033158.4 | RNF180/NM001113561.2 |
| ART4/NM021071.2 | HYAL3/NM001200029.1 | RNF181/NM016494.3 |
| ARTN/NM001136215.1 | HYAL3/NM003549.3 | RNF182/NM001165033.1 |
| ARV1/NM022786.1 | HYAL4/NM012269.2 | RNF182/NM001165034.1 |
| ARVCF/NM001670.2 | HYDIN/NM001270974.2 | RNF182/NM152737.3 |
| AS3MT/NM020682.3 | HYI/NM001190880.2 | RNF183/NM145051.3 |
| ASAH1/NM004315.5 | HYKK/NM001013619.3 | RNF185/NM152267.3 |
| ASAH2/NM019893.2 | HYLS1/NM001134793.1 | RNF186/NM019062.1 |
| ASAH2B/NM001321958.1 | HYLS1/NM145014.2 | RNF187/NM001010858.2 |
| ASAP1/NM018482.3 | HYOU1/NM006389.4 | RNF19A/NM001280539.1 |
| ASAP2/NM003887.2 | HYPK/NM016400.3 | RNF19A/NM015435.4 |
| ASAP3/NM017707.3 | IAH1/NM001039613.2 | RNF19A/NM183419.3 |
| ASB10/NM001142459.1 | IAPP/NM000415.2 | RNF19B/NM153341.3 |
| ASB13/NM024701.3 | IARS/NM013417.3 | RNF2/NM007212.3 |
| ASB14/NM001142733.2 | IARS2/NM018060.3 | RNF20/NM019592.6 |
| ASB15/NM001290258.1 | IBA57/NM001010867.3 | RNF207/NM207396.2 |
| ASB15/NM080928.4 | IBSP/NM004967.3 | RNF208/NM031297.4 |
| ASB16/NM080863.4 | IBTK/NM015525.3 | RNF212/NM194439.4 |
| ASB17/NM080868.2 | ICA1/NM001136020.2 | RNF212B/NM001282322.1 |
| ASB18/NM212556.2 | ICA1/NM004968.3 | RNF213/NM001256071.2 |
| ASB2/NM001202429.1 | ICA1/NM022307.2 | RNF214/NM001077239.1 |
| ASB3/NM016115.4 | ICA1L/NM001288623.1 | RNF214/NM207343.3 |
| ASB4/NM016116.2 | ICA1L/NM138468.5 | RNF215/NM001017981.1 |
| ASB7/NM198243.2 | ICAM1/NM000201.2 | RNF217/NM001286398.2 |
| ASB8/NM001319296.1 | ICAM2/NM000873.3 | RNF219/NM024546.3 |
| ASCC1/NM001198798.2 | ICAM2/NM001099786.1 | RNF220/NM018150.3 |
| ASCC1/NM001198800.2 | ICAM3/NM002162.4 | RNF222/NM001146684.2 |
| ASCC2/NM032204.4 | ICAM4/NM001544.4 | RNF223/NM001205252.1 |
| ASCC3/NM006828.3 | ICAM5/NM003259.3 | RNF224/NM001190228.1 |
| ASCL1/NM004316.3 | ICE1/NM015325.2 | RNF225/NM001195135.1 |
| ASCL2/NM005170.2 | ICE2/NM024611.5 | RNF24/NM007219.4 |
| ASCL3/NM020646.2 | ICK/NM016513.4 | RNF25/NM022453.2 |
| ASCL4/NM203436.2 | ICMT/NM012405.3 | RNF26/NM032015.4 |
| ASCL5/NM001270601.1 | ICOS/NM012092.3 | RNF31/NM017999.4 |
| ASF1A/NM014034.2 | ICOSLG/NM015259.5 | RNF32/NM001184996.1 |
| ASF1B/NM018154.2 | ICT1/NM001545.2 | RNF32/NM001184997.1 |
| ASGR1/NM001671.4 | ID1/NM002165.3 | RNF32/NM030936.3 |
| ASGR2/NM080913.3 | ID2/NM002166.4 | RNF34/NM194271.2 |
| ASH1L/NM018489.2 | ID3/NM002167.4 | RNF38/NM022781.4 |
| ASH2L/NM004674.4 | ID4/NM001546.3 | RNF39/NM025236.3 |
| ASIC1/NM001095.3 | IDE/NM004969.3 | RNF40/NM001286572.2 |
| ASIC2/NM183377.1 | IDH1/NM001282386.1 | RNF40/NM014771.3 |
| ASIC3/NM004769.3 | IDH1/NM001282387.1 | RNF41/NM001242826.1 |
| ASIC4/NM182847.2 | IDH1/NM005896.3 | RNF41/NM005785.3 |
| ASIC5/NM017419.2 | IDH2/NM002168.3 | RNF41/NM194359.2 |
| ASIP/NM001672.2 | IDH3A/NM005530.2 | RNF43/NM017763.5 |
| ASL/NM000048.3 | IDH3B/NM006899.4 | RNF44/NM014901.4 |
| ASL/NM001024943.1 | IDI1/NM001317955.1 | RNF5/NM006913.3 |
| ASNS/NM133436.3 | IDI1/NM001317956.1 | RNF6/NM005977.3 |
| ASNS/NM183356.3 | IDI2/NM033261.2 | RNF6/NM183043.2 |
| ASNSD1/NM019048.2 | IDNK/NM001001551.3 | RNF6/NM183044.2 |
| ASPA/NM000049.2 | IDO1/NM002164.5 | RNF7/NM014245.4 |
| ASPA/NM001128085.1 | IDO2/NM194294.2 | RNF8/NM003958.3 |
| ASPDH/NM001114598.1 | IDUA/NM000203.4 | RNFT1/NM016125.3 |
| ASPG/NM001080464.2 | IER2/NM004907.2 | RNFT2/NM001109903.1 |
| ASPH/NM004318.3 | IER3/NM003897.3 | RNGTT/NM003800.4 |
| ASPHD1/NM181718.3 | IER3IP1/NM016097.4 | RNH1/NM002939.3 |
| ASPHD2/NM020437.4 | IER5/NM016545.4 | RNH1/NM203387.2 |
| ASPM/NM018136.4 | IER5L/NM203434.2 | RNLS/NM001031709.2 |
| ASPN/NM017680.4 | IFFO1/NM080730.4 | RNPC3/NM017619.3 |
| ASPRV1/NM152792.2 | IFFO2/NM001136265.1 | RNPEP/NM020216.3 |
| ASPSCR1/NM024083.3 | IFI16/NM005531.2 | RNPEPL1/NM018226.4 |
| ASRGL1/NM025080.3 | IFI27/NM001288957.1 | RNPS1/NM001286625.1 |
| ASS1/NM054012.3 | IFI27/NM001288958.1 | RNPS1/NM006711.4 |
| ASTE1/NM014065.3 | IFI27L1/NM206949.2 | RNPS1/NM080594.3 |
| ASTL/NM001002036.3 | IFI27L2/NM032036.2 | ROBO1/NM133631.3 |
| ASTN1/NM004319.2 | IFI30/NM006332.4 | ROBO3/NM022370.3 |
| ASTN2/NM014010.4 | IFI35/NM005533.4 | ROBO4/NM019055.5 |
| ASXL1/NM015338.5 | IFI44/NM006417.4 | ROCK1/NM005406.2 |
| ASXL2/NM018263.4 | IFI44L/NM006820.3 | ROCK2/NM004850.4 |
| ASXL3/NM030632.2 | IFI6/NM022873.2 | ROGDI/NM024589.2 |
| ATAD1/NM032810.3 | IFIH1/NM022168.3 | ROM1/NM000327.3 |
| ATAD2/NM014109.3 | IFIT1/NM001548.4 | ROMO1/NM080748.2 |
| ATAD2B/NM017552.3 | IFIT1B/NM001010987.2 | ROPN1/NM001317774.1 |
| ATAD3A/NM018188.3 | IFIT2/NM001547.4 | ROPN1/NM017578.3 |
| ATAD3B/NM031921.5 | IFIT3/NM001031683.3 | ROPN1B/NM001012337.2 |
| ATAD3C/NM001039211.2 | IFIT3/NM001549.5 | ROPN1L/NM001201466.1 |
| ATAD5/NM024857.4 | IFIT5/NM012420.2 | ROR1/NM005012.3 |
| ATAT1/NM024909.3 | IFITM1/NM003641.3 | ROR2/NM004560.3 |
| ATCAY/NM033064.4 | IFITM10/NM001170820.3 | RORA/NM134261.2 |
| ATE1/NM001001976.2 | IFITM2/NM006435.2 | RORB/NM006914.3 |
| ATF1/NM005171.4 | IFITM3/NM021034.2 | RORC/NM005060.3 |
| ATF3/NM001030287.3 | IFITM5/NM001025295.2 | ROS1/NM002944.2 |
| ATF3/NM001674.3 | IFNA1/NM024013.2 | RP1/NM006269.1 |
| ATF4/NM001675.4 | IFNA10/NM002171.2 | RP1L1/NM178857.5 |
| ATF4/NM182810.2 | IFNA13/NM006900.3 | RP9/NM203288.1 |
| ATF5/NM001193646.1 | IFNA14/NM002172.2 | RPA1/NM002945.3 |
| ATF5/NM001290746.1 | IFNA16/NM002173.3 | RPA2/NM002946.4 |
| ATF5/NM012068.5 | IFNA17/NM021268.2 | RPA3/NM002947.4 |
| ATF6/NM007348.3 | IFNA2/NM000605.3 | RPAIN/NM001033002.3 |
| ATF6B/NM004381.4 | IFNA21/NM002175.2 | RPAP1/NM015540.3 |
| ATF7/NM006856.2 | IFNA4/NM021068.2 | RPAP2/NM024813.2 |
| ATF7IP/NM018179.4 | IFNA5/NM002169.2 | RPAP3/NM024604.2 |
| ATF7IP2/NM024997.3 | IFNA6/NM021002.2 | RPE/NM199229.2 |
| ATG10/NM031482.4 | IFNA7/NM021057.2 | RPE65/NM000329.2 |
| ATG101/NM001098673.1 | IFNA8/NM002170.3 | RPEL1/NM001143909.1 |
| ATG12/NM004707.3 | IFNAR1/NM000629.2 | RPF1/NM025065.6 |
| ATG13/NM014741.4 | IFNAR2/NM207585.2 | RPF2/NM032194.2 |
| ATG14/NM014924.4 | IFNB1/NM002176.3 | RPGRIP1/NM020366.3 |
| ATG16L2/NM033388.1 | IFNE/NM176891.4 | RPGRIP1L/NM001127897.2 |
| ATG2A/NM015104.2 | IFNG/NM000619.2 | RPH3A/NM014954.3 |
| ATG2B/NM018036.5 | IFNGR1/NM000416.2 | RPH3AL/NM006987.3 |
| ATG3/NM022488.4 | IFNGR2/NM005534.3 | RPIA/NM144563.2 |
| ATG4B/NM013325.4 | IFNK/NM020124.2 | RPL10A/NM007104.4 |
| ATG4C/NM178221.2 | IFNL1/NM172140.1 | RPL10L/NM080746.2 |
| ATG4D/NM032885.5 | IFNL2/NM172138.1 | RPL11/NM000975.3 |
| ATG5/NM001286106.1 | IFNL3/NM172139.2 | RPL13/NM000977.3 |
| ATG5/NM004849.3 | IFNLR1/NM170743.3 | RPL13/NM033251.2 |
| ATG9A/NM001077198.2 | IFNW1/NM002177.2 | RPL13A/NM012423.3 |
| ATG9B/NM001317056.1 | IFRD1/NM001007245.2 | RPL14/NM001034996.2 |
| ATHL1/NM025092.4 | IFRD1/NM001550.3 | RPL14/NM003973.4 |
| ATIC/NM004044.6 | IFRD2/NM006764.4 | RPL15/NM001253380.1 |
| ATL1/NM015915.4 | IFT122/NM052985.3 | RPL15/NM001253382.1 |
| ATL2/NM022374.3 | IFT140/NM014714.3 | RPL15/NM001253383.1 |
| ATL3/NM015459.4 | IFT172/NM015662.2 | RPL15/NM002948.3 |
| ATM/NM000051.3 | IFT20/NM001267775.1 | RPL17-C18orf32/NM001199355.1 |
| ATMIN/NM015251.2 | IFT22/NM022777.3 | RPL17/NM000985.4 |
| ATN1/NM001007026.1 | IFT27/NM006860.4 | RPL17/NM001199342.1 |
| ATN1/NM001940.3 | IFT43/NM001102564.1 | RPL18/NM000979.3 |
| ATOH1/NM005172.1 | IFT46/NM020153.3 | RPL18A/NM000980.3 |
| ATOH7/NM145178.3 | IFT57/NM018010.3 | RPL19/NM000981.3 |
| ATOH8/NM032827.6 | IFT74/NM001099222.1 | RPL21/NM000982.3 |
| ATOX1/NM004045.3 | IFT74/NM001099223.1 | RPL22/NM000983.3 |
| ATP10A/NM024490.3 | IFT80/NM020800.2 | RPL22L1/NM001099645.1 |
| ATP10B/NM025153.2 | IFT81/NM001143779.1 | RPL23/NM000978.3 |
| ATP10D/NM020453.3 | IGDCC3/NM004884.3 | RPL23A/NM000984.5 |
| ATP11A/NM015205.2 | IGDCC4/NM020962.2 | RPL24/NM000986.3 |
| ATP11AUN/NM207440.2 | IGF1/NM000618.4 | RPL26/NM000987.3 |
| ATP11B/NM014616.2 | IGF1R/NM000875.4 | RPL26/NM001315530.1 |
| ATP12A/NM001676.5 | IGF2/NM000612.5 | RPL26L1/NM001317980.1 |
| ATP13A1/NM020410.2 | IGF2/NM001007139.5 | RPL26L1/NM001317981.1 |
| ATP13A2/NM001141973.2 | IGF2/NM001291861.2 | RPL26L1/NM001317982.1 |
| ATP13A3/NM024524.3 | IGF2/NM001291862.2 | RPL26L1/NM016093.3 |
| ATP13A4/NM032279.3 | IGF2BP2/NM006548.4 | RPL27/NM000988.3 |
| ATP13A5/NM198505.2 | IGF2BP3/NM006547.2 | RPL27A/NM000990.4 |
| ATP1A1/NM000701.7 | IGF2R/NM000876.3 | RPL28/NM000991.4 |
| ATP1A2/NM000702.3 | IGFALS/NM004970.2 | RPL29/NM000992.2 |
| ATP1A3/NM152296.4 | IGFBP1/NM000596.2 | RPL3/NM000967.3 |
| ATP1A4/NM144699.3 | IGFBP2/NM000597.2 | RPL30/NM000989.3 |
| ATP1B1/NM001677.3 | IGFBP3/NM000598.4 | RPL31/NM000993.4 |
| ATP1B2/NM001678.4 | IGFBP4/NM001552.2 | RPL32/NM001007073.1 |
| ATP23/NM033276.3 | IGFBP5/NM000599.3 | RPL32/NM001007074.1 |
| ATP2A1/NM004320.4 | IGFBP6/NM002178.2 | RPL34/NM001319234.1 |
| ATP2A2/NM170665.3 | IGFBP7/NM001553.2 | RPL34/NM001319235.1 |
| ATP2A3/NM005173.3 | IGFBPL1/NM001007563.2 | RPL34/NM001319236.1 |
| ATP2B1/NM001682.2 | IGFL1/NM198541.1 | RPL35/NM007209.3 |
| ATP2B2/NM001683.3 | IGFL2/NM001002915.2 | RPL35A/NM000996.2 |
| ATP2B4/NM001684.4 | IGFL3/NM207393.1 | RPL35A/NM001316311.1 |
| ATP2C1/NM001001486.1 | IGFL4/NM001002923.1 | RPL36/NM015414.3 |
| ATP2C2/NM014861.3 | IGFLR1/NM024660.2 | RPL36/NM033643.2 |
| ATP4A/NM000704.2 | IGFN1/NM001164586.1 | RPL36AL/NM001001.4 |
| ATP4B/NM000705.3 | IGIP/NM001007189.1 | RPL37/NM000997.4 |
| ATP5A1/NM001001937.1 | IGLL1/NM020070.3 | RPL37A/NM000998.4 |
| ATP5A1/NM004046.5 | IGLL5/NM001178126.1 | RPL38/NM001035258.1 |
| ATP5B/NM001686.3 | IGLON5/NM001101372.1 | RPL39L/NM052969.1 |
| ATP5C1/NM005174.3 | IGSF10/NM178822.4 | RPL3L/NM005061.2 |
| ATP5D/NM001001975.1 | IGSF11/NM152538.2 | RPL4/NM000968.3 |
| ATP5E/NM006886.3 | IGSF21/NM032880.4 | RPL41/NM021104.1 |
| ATP5F1/NM001688.4 | IGSF22/NM173588.3 | RPL5/NM000969.3 |
| ATP5G1/NM001002027.1 | IGSF3/NM001542.3 | RPL6/NM001320137.1 |
| ATP5G3/NM001002258.4 | IGSF5/NM001080444.1 | RPL6/NM001320140.1 |
| ATP5G3/NM001689.4 | IGSF6/NM005849.3 | RPL6/NM001320141.1 |
| ATP5H/NM006356.2 | IGSF8/NM001206665.2 | RPL7/NM000971.3 |
| ATP5I/NM007100.3 | IGSF8/NM052868.5 | RPL7A/NM000972.2 |
| ATP5J2-PTCD1/NM001198879.1 | IGSF9/NM001135050.1 | RPL7L1/NM198486.3 |
| ATP5L/NM006476.4 | IGSF9B/NM001277285.1 | RPL8/NM000973.4 |
| ATP5L2/NM001165877.1 | IHH/NM002181.3 | RPL8/NM001317782.1 |
| ATP5O/NM001697.2 | IK/NM006083.3 | RPL9/NM001024921.3 |
| ATP5SL/NM018035.2 | IKBIP/NM153687.3 | RPLP0/NM053275.3 |
| ATP6V0A1/NM005177.3 | IKBKAP/NM003640.4 | RPLP1/NM001003.2 |
| ATP6V0A2/NM012463.3 | IKBKB/NM001556.2 | RPLP2/NM001004.3 |
| ATP6V0A4/NM020632.2 | IKBKE/NM014002.3 | RPN1/NM002950.3 |
| ATP6V0A4/NM130841.2 | IKZF1/NM006060.5 | RPN2/NM002951.4 |
| ATP6V0B/NM004047.4 | IKZF2/NM016260.2 | RPP14/NM007042.4 |
| ATP6V0C/NM001198569.1 | IKZF3/NM012481.4 | RPP21/NM024839.2 |
| ATP6V0C/NM001694.3 | IKZF4/NM022465.3 | RPP25/NM017793.2 |
| ATP6V0D1/NM004691.4 | IKZF5/NM001271840.1 | RPP25L/NM148179.2 |
| ATP6V0D2/NM152565.1 | IL10/NM000572.2 | RPP30/NM006413.4 |
| ATP6V0E2/NM145230.3 | IL10RA/NM001558.3 | RPP38/NM006414.4 |
| ATP6V1A/NM001690.3 | IL10RB/NM000628.4 | RPP40/NM006638.3 |
| ATP6V1B1/NM001692.3 | IL11/NM000641.3 | RPRD1A/NM018170.4 |
| ATP6V1B2/NM001693.3 | IL11RA/NM001142784.2 | RPRD1B/NM021215.3 |
| ATP6V1C1/NM001695.4 | IL12A/NM000882.3 | RPRD2/NM015203.4 |
| ATP6V1C2/NM144583.3 | IL12B/NM002187.2 | RPRM/NM019845.2 |
| ATP6V1D/NM015994.3 | IL12RB1/NM005535.2 | RPRML/NM203400.4 |
| ATP6V1E1/NM001696.3 | IL12RB2/NM001559.2 | RPS10-NUDT3/NM001202470.2 |
| ATP6V1E2/NM001318063.1 | IL13/NM002188.2 | RPS10/NM001203245.2 |
| ATP6V1F/NM004231.3 | IL15/NM000585.4 | RPS11/NM001015.4 |
| ATP6V1G1/NM004888.3 | IL15RA/NM002189.3 | RPS12/NM001016.3 |
| ATP6V1G2/NM130463.3 | IL16/NM172217.3 | RPS13/NM001017.2 |
| ATP6V1G3/NM133262.2 | IL17A/NM002190.2 | RPS14/NM001025071.1 |
| ATP6V1H/NM015941.3 | IL17B/NM014443.2 | RPS15/NM001018.4 |
| ATP6V1H/NM213620.2 | IL17C/NM013278.3 | RPS15A/NM001019.4 |
| ATP7B/NM000053.3 | IL17D/NM138284.1 | RPS16/NM001020.5 |
| ATP8A1/NM006095.2 | IL17F/NM052872.3 | RPS17/NM001021.4 |
| ATP8A2/NM016529.5 | IL17RB/NM018725.3 | RPS18/NM022551.2 |
| ATP8B1/NM005603.4 | IL17RC/NM153461.3 | RPS19/NM001022.3 |
| ATP8B3/NM138813.3 | IL17RE/NM153480.1 | RPS19/NM001321483.1 |
| ATP8B4/NM024837.3 | IL18/NM001562.3 | RPS19/NM001321484.1 |
| ATP9B/NM198531.4 | IL18BP/NM001039660.1 | RPS19BP1/NM194326.3 |
| ATPAF1/NM022745.4 | IL18BP/NM001145057.1 | RPS2/NM002952.3 |
| ATPIF1/NM016311.4 | IL18BP/NM173042.2 | RPS20/NM001023.3 |
| ATR/NM001184.3 | IL18R1/NM003855.3 | RPS21/NM001024.3 |
| ATRAID/NM080592.3 | IL18RAP/NM003853.3 | RPS23/NM001025.4 |
| ATRIP/NM130384.2 | IL19/NM153758.2 | RPS24/NM001026.4 |
| ATRN/NM139321.2 | IL1A/NM000575.4 | RPS25/NM001028.2 |
| ATXN1/NM000332.3 | IL1B/NM000576.2 | RPS26/NM001029.3 |
| ATXN10/NM013236.3 | IL1F10/NM032556.5 | RPS27/NM001030.4 |
| ATXN1L/NM001137675.3 | IL1F10/NM173161.2 | RPS27A/NM001135592.2 |
| ATXN2/NM002973.3 | IL1R1/NM000877.3 | RPS27A/NM001177413.1 |
| ATXN2L/NM148414.2 | IL1R2/NM004633.3 | RPS27A/NM002954.5 |
| ATXN3/NM004993.5 | IL1RL1/NM016232.4 | RPS27L/NM015920.3 |
| ATXN7/NM000333.3 | IL1RL2/NM003854.2 | RPS28/NM001031.4 |
| ATXN7L1/NM020725.1 | IL1RN/NM000577.4 | RPS29/NM001032.4 |
| ATXN7L2/NM153340.4 | IL2/NM000586.3 | RPS3/NM001005.4 |
| ATXN7L3/NM001098833.1 | IL20/NM018724.3 | RPS3A/NM001006.4 |
| ATXN7L3B/NM001136262.1 | IL20RA/NM014432.3 | RPS5/NM001009.3 |
| AUH/NM001698.2 | IL20RB/NM144717.3 | RPS6/NM001010.2 |
| AUNIP/NM024037.2 | IL21/NM021803.3 | RPS6KA1/NM002953.3 |
| AUP1/NM181575.4 | IL21R/NM021798.3 | RPS6KA2/NM021135.5 |
| AURKA/NM198433.2 | IL21R/NM181078.2 | RPS6KA4/NM003942.2 |
| AURKAIP1/NM001127229.1 | IL22/NM020525.4 | RPS6KA5/NM004755.3 |
| AURKAIP1/NM001127230.1 | IL22RA1/NM021258.3 | RPS6KB1/NM003161.3 |
| AURKAIP1/NM017900.2 | IL22RA2/NM181309.1 | RPS6KB2/NM003952.2 |
| AURKB/NM004217.3 | IL23A/NM016584.2 | RPS6KC1/NM012424.4 |
| AURKC/NM001015878.1 | IL23R/NM144701.2 | RPS6KL1/NM031464.4 |
| AUTS2/NM015570.3 | IL24/NM006850.3 | RPS7/NM001011.3 |
| AVEN/NM020371.2 | IL25/NM022789.3 | RPS8/NM001012.1 |
| AVIL/NM006576.3 | IL26/NM018402.1 | RPS9/NM001321702.1 |
| AVL9/NM015060.2 | IL27/NM145659.3 | RPS9/NM001321704.1 |
| AVP/NM000490.4 | IL27RA/NM004843.3 | RPSA/NM002295.5 |
| AVPI1/NM021732.2 | IL2RA/NM000417.2 | RPTN/NM001122965.1 |
| AVPR1A/NM000706.4 | IL2RB/NM000878.3 | RPTOR/NM020761.2 |
| AVPR1B/NM000707.3 | IL3/NM000588.3 | RPUSD1/NM058192.2 |
| AXDND1/NM144696.5 | IL31/NM001014336.1 | RPUSD2/NM152260.2 |
| AXIN1/NM003502.3 | IL31RA/NM139017.5 | RPUSD4/NM032795.2 |
| AXIN2/NM004655.3 | IL32/NM001012632.1 | RRAD/NM001128850.1 |
| AXL/NM021913.4 | IL32/NM004221.4 | RRAGA/NM006570.4 |
| AZGP1/NM001185.3 | IL33/NM033439.3 | RRAGC/NM022157.3 |
| AZI2/NM022461.4 | IL34/NM001172772.1 | RRAGD/NM021244.4 |
| AZIN1/NM015878.5 | IL34/NM152456.2 | RRAS/NM006270.4 |
| AZIN2/NM001293562.1 | IL36A/NM014440.2 | RRAS2/NM012250.5 |
| AZU1/NM001700.4 | IL36B/NM173178.2 | RRBP1/NM001042576.1 |
| B2M/NM004048.2 | IL37/NM014439.3 | RREB1/NM001003699.3 |
| B3GALNT1/NM001038628.1 | IL4/NM000589.3 | RRH/NM006583.2 |
| B3GALNT1/NM033169.2 | IL4I1/NM001258018.1 | RRM1/NM001033.4 |
| B3GALNT2/NM152490.4 | IL4R/NM001257406.1 | RRM2/NM001034.3 |
| B3GALT1/NM020981.3 | IL5RA/NM000564.4 | RRM2B/NM015713.4 |
| B3GALT2/NM003783.3 | IL6/NM000600.4 | RRN3/NM018427.4 |
| B3GALT4/NM003782.3 | IL6R/NM000565.3 | RRNAD1/NM015997.3 |
| B3GALT5/NM001278650.1 | IL7/NM000880.3 | RRP1/NM003683.5 |
| B3GALT5/NM033170.2 | IL7R/NM002185.3 | RRP12/NM015179.3 |
| B3GALT6/NM080605.3 | IL9/NM000590.1 | RRP15/NM016052.3 |
| B3GAT1/NM054025.2 | ILDR1/NM175924.3 | RRP1B/NM015056.2 |
| B3GAT2/NM080742.2 | ILDR2/NM199351.2 | RRP36/NM033112.2 |
| B3GAT3/NM012200.3 | ILF2/NM004515.3 | RRP7A/NM015703.4 |
| B3GLCT/NM194318.3 | ILF3/NM012218.3 | RRP8/NM015324.3 |
| B3GNT2/NM001319075.1 | ILK/NM001014794.2 | RRP9/NM004704.4 |
| B3GNT2/NM006577.5 | ILK/NM001014795.2 | RRS1/NM015169.3 |
| B3GNT3/NM014256.3 | ILKAP/NM030768.2 | RSAD1/NM018346.2 |
| B3GNT4/NM030765.2 | IMMP1L/NM144981.2 | RSAD2/NM080657.4 |
| B3GNT5/NM032047.4 | IMMP2L/NM001244606.1 | RSBN1/NM018364.4 |
| B3GNT6/NM138706.4 | IMMP2L/NM032549.3 | RSBN1L/NM198467.2 |
| B3GNT7/NM145236.2 | IMMT/NM006839.2 | RSC1A1/NM006511.2 |
| B3GNT8/NM198540.2 | IMP3/NM018285.3 | RSG1/NM030907.3 |
| B3GNT9/NM033309.2 | IMP4/NM033416.2 | RSL1D1/NM015659.2 |
| B3GNTL1/NM001009905.2 | IMPA1/NM005536.3 | RSL24D1/NM016304.2 |
| B4GALNT2/NM153446.2 | IMPA2/NM014214.2 | RSPH1/NM080860.3 |
| B4GALNT3/NM173593.3 | IMPACT/NM018439.3 | RSPH10B/NM173565.3 |
| B4GALNT4/NM178537.4 | IMPAD1/NM017813.4 | RSPH10B2/NM001099697.1 |
| B4GALT1/NM001497.3 | IMPDH1/NM000883.3 | RSPH14/NM014433.2 |
| B4GALT2/NM001005417.2 | IMPDH2/NM000884.2 | RSPH3/NM031924.4 |
| B4GALT2/NM003780.4 | IMPG1/NM001563.3 | RSPH4A/NM001010892.2 |
| B4GALT3/NM001199873.1 | IMPG2/NM016247.3 | RSPH6A/NM030785.3 |
| B4GALT5/NM004776.3 | INA/NM032727.3 | RSPH9/NM152732.4 |
| B4GALT6/NM004775.3 | INAFM1/NM178511.5 | RSPO1/NM001038633.3 |
| B4GALT7/NM007255.2 | INAFM2/NM001301268.1 | RSPO2/NM178565.4 |
| B4GAT1/NM006876.2 | INCA1/NM213726.2 | RSPO3/NM032784.4 |
| B9D1/NM015681.4 | INCENP/NM020238.2 | RSPO4/NM001029871.3 |
| B9D2/NM030578.3 | INF2/NM022489.3 | RSRC1/NM016625.3 |
| BAALC/NM024812.2 | ING1/NM198219.2 | RSRC2/NM023012.5 |
| BAAT/NM001127610.1 | ING2/NM001564.3 | RSRP1/NM001321772.1 |
| BAAT/NM001701.3 | ING3/NM019071.2 | RSRP1/NM020317.4 |
| BABAM1/NM001288756.1 | ING4/NM016162.3 | RSU1/NM012425.3 |
| BACE1/NM012104.4 | ING5/NM032329.4 | RTBDN/NM031429.2 |
| BACE2/NM012105.4 | INHA/NM002191.3 | RTCA/NM003729.3 |
| BACH1/NM001186.3 | INHBA/NM002192.2 | RTCB/NM014306.4 |
| BACH1/NM206866.2 | INHBB/NM002193.2 | RTEL1/NM032957.4 |
| BACH2/NM021813.3 | INHBC/NM005538.3 | RTF1/NM015138.4 |
| BAD/NM004322.3 | INHBE/NM031479.3 | RTFDC1/NM016407.4 |
| BAG1/NM004323.5 | INIP/NM021218.1 | RTKN/NM033046.2 |
| BAG2/NM004282.3 | INMT/NM006774.4 | RTKN2/NM145307.3 |
| BAG3/NM004281.3 | INO80/NM017553.2 | RTL1/NM001134888.2 |
| BAG4/NM004874.3 | INO80B/NM031288.3 | RTN1/NM206852.2 |
| BAG6/NM001098534.1 | INO80C/NM194281.3 | RTN2/NM206901.2 |
| BAG6/NM080703.2 | INO80D/NM017759.4 | RTN4/NM007008.2 |
| BAGE2/NM182482.2 | INO80E/NM173618.2 | RTN4IP1/NM032730.5 |
| BAHCC1/NM001291324.1 | INPP1/NM002194.3 | RTN4R/NM023004.5 |
| BAHD1/NM014952.4 | INPP4A/NM001566.2 | RTN4RL1/NM178568.3 |
| BAIAP2L1/NM018842.4 | INPP4B/NM003866.2 | RTN4RL2/NM178570.2 |
| BAIAP2L2/NM025045.5 | INPP5A/NM005539.4 | RTP1/NM153708.2 |
| BAIAP3/NM003933.4 | INPP5B/NM005540.2 | RTP2/NM001004312.2 |
| BAK1/NM001188.3 | INPP5D/NM001017915.2 | RTP3/NM031440.1 |
| BAMBI/NM012342.2 | INPP5E/NM019892.5 | RTP4/NM022147.2 |
| BANF1/NM001143985.1 | INPP5F/NM014937.3 | RTP5/NM173821.2 |
| BANF2/NM001014977.3 | INPP5J/NM001002837.2 | RTTN/NM173630.3 |
| BANK1/NM017935.4 | INPP5K/NM016532.3 | RUBCN/NM014687.2 |
| BANP/NM001173542.1 | INPPL1/NM001567.3 | RUFY1/NM001040452.2 |
| BAP1/NM004656.3 | INSC/NM001031853.4 | RUFY3/NM001037442.3 |
| BARD1/NM000465.3 | INSIG1/NM005542.4 | RUFY4/NM198483.3 |
| BARHL1/NM020064.3 | INSIG2/NM001321329.1 | RUNDC1/NM173079.3 |
| BARHL2/NM020063.1 | INSIG2/NM016133.3 | RUNDC3A/NM001144825.1 |
| BARX1/NM021570.3 | INSL3/NM005543.3 | RUNDC3B/NM001134405.1 |
| BARX2/NM003658.4 | INSL4/NM002195.1 | RUNX1/NM001754.4 |
| BATF/NM006399.3 | INSL5/NM005478.4 | RUNX1T1/NM001198627.1 |
| BATF2/NM138456.3 | INSL6/NM007179.2 | RUNX1T1/NM001198628.1 |
| BATF3/NM018664.2 | INSM1/NM002196.2 | RUNX1T1/NM001198630.1 |
| BAX/NM138761.3 | INSM2/NM032594.3 | RUNX1T1/NM175634.2 |
| BAZ1A/NM013448.2 | INSR/NM000208.3 | RUNX2/NM001024630.3 |
| BAZ1B/NM032408.3 | INSRR/NM014215.2 | RUNX3/NM004350.2 |
| BAZ2A/NM013449.3 | INTS1/NM001080453.2 | RUSC1-AS1/NM001039517.1 |
| BAZ2B/NM013450.3 | INTS10/NM018142.2 | RUSC1/NM001278230.1 |
| BBC3/NM014417.4 | INTS12/NM020395.3 | RUSC1/NM014328.4 |
| BBIP1/NM001243783.1 | INTS2/NM020748.2 | RUSC2/NM001135999.1 |
| BBOF1/NM025057.2 | INTS3/NM023015.3 | RUSC2/NM014806.3 |
| BBOX1/NM003986.2 | INTS4/NM033547.3 | RUVBL1/NM003707.2 |
| BBS10/NM024685.3 | INTS5/NM030628.1 | RUVBL2/NM006666.2 |
| BBS12/NM152618.2 | INTS6/NM012141.2 | RWDD2B/NM001320724.1 |
| BBS2/NM031885.3 | INTS7/NM015434.3 | RWDD3/NM015485.4 |
| BBS4/NM033028.4 | INTS8/NM017864.3 | RWDD4/NM152682.3 |
| BBS5/NM152384.2 | INTS9/NM018250.3 | RXFP1/NM021634.3 |
| BBS7/NM176824.2 | INTU/NM015693.3 | RXFP2/NM130806.3 |
| BBS9/NM001033605.1 | INVS/NM014425.4 | RXFP3/NM016568.3 |
| BBX/NM020235.6 | IP6K1/NM001242829.1 | RXFP4/NM181885.2 |
| BCAM/NM005581.4 | IP6K1/NM153273.3 | RXRA/NM001291920.1 |
| BCAP29/NM018844.3 | IP6K2/NM016291.3 | RXRB/NM021976.4 |
| BCAR1/NM014567.3 | IPCEF1/NM015553.2 | RXRG/NM006917.4 |
| BCAR3/NM001261408.1 | IPMK/NM152230.4 | RYBP/NM012234.6 |
| BCAR3/NM001261409.1 | IPO11/NM016338.4 | RYK/NM001005861.2 |
| BCAR3/NM003567.3 | IPO13/NM014652.3 | RYR1/NM000540.2 |
| BCAS1/NM003657.3 | IPO4/NM024658.3 | RYR2/NM001035.2 |
| BCAS2/NM005872.2 | IPO5/NM002271.4 | RYR3/NM001036.4 |
| BCAS3/NM017679.3 | IPO7/NM006391.2 | S100A1/NM006271.1 |
| BCAS4/NM198799.3 | IPO8/NM006390.3 | S100A10/NM002966.2 |
| BCAT1/NM005504.6 | IPO9/NM018085.4 | S100A11/NM005620.1 |
| BCAT2/NM001190.3 | IPP/NM005897.2 | S100A12/NM005621.1 |
| BCCIP/NM016567.3 | IPPK/NM022755.5 | S100A13/NM001024210.1 |
| BCDIN3D/NM181708.2 | IQCA1/NM024726.4 | S100A13/NM001024211.1 |
| BCHE/NM000055.2 | IQCA1L/NM001304419.1 | S100A13/NM001024212.1 |
| BCKDHA/NM000709.3 | IQCB1/NM001023570.3 | S100A13/NM001024213.1 |
| BCKDHB/NM000056.4 | IQCC/NM018134.2 | S100A13/NM005979.2 |
| BCKDK/NM005881.3 | IQCD/NM138451.2 | S100A14/NM020672.2 |
| BCL10/NM003921.4 | IQCE/NM152558.4 | S100A16/NM001317007.1 |
| BCL11A/NM022893.3 | IQCF1/NM152397.2 | S100A16/NM080388.2 |
| BCL11B/NM138576.3 | IQCF3/NM001207023.1 | S100A2/NM005978.3 |
| BCL2/NM000633.2 | IQCF5/NM001145059.1 | S100A3/NM002960.1 |
| BCL2A1/NM004049.3 | IQCG/NM001134435.2 | S100A4/NM019554.2 |
| BCL2L1/NM001317919.1 | IQCG/NM032263.4 | S100A5/NM002962.1 |
| BCL2L1/NM001322240.1 | IQCH/NM022784.2 | S100A6/NM014624.3 |
| BCL2L1/NM001322242.1 | IQCJ-SCHIP1/NM001197113.1 | S100A7/NM002963.3 |
| BCL2L10/NM020396.3 | IQCK/NM153208.2 | S100A7A/NM176823.3 |
| BCL2L11/NM006538.4 | IQGAP1/NM003870.3 | S100A7L2/NM001045479.1 |
| BCL2L12/NM138639.1 | IQGAP2/NM006633.3 | S100A8/NM001319201.1 |
| BCL2L13/NM015367.3 | IQGAP3/NM178229.4 | S100A8/NM002964.4 |
| BCL2L14/NM138722.1 | IQSEC1/NM014869.6 | S100A9/NM002965.3 |
| BCL2L14/NM138723.1 | IQSEC3/NM015232.1 | S100B/NM006272.2 |
| BCL2L15/NM001010922.2 | IQUB/NM001282855.1 | S100P/NM005980.2 |
| BCL2L2/NM001199839.1 | IQUB/NM178827.4 | S100PBP/NM001256121.1 |
| BCL2L2/NM004050.4 | IRAK1BP1/NM001010844.3 | S100PBP/NM022753.3 |
| BCL3/NM005178.4 | IRAK2/NM001570.3 | S100Z/NM130772.3 |
| BCL6/NM001130845.1 | IRAK3/NM007199.2 | S1PR1/NM001320730.1 |
| BCL6/NM001706.4 | IRAK4/NM016123.3 | S1PR2/NM004230.3 |
| BCL6B/NM181844.3 | IREB2/NM004136.3 | S1PR3/NM005226.3 |
| BCL7B/NM001707.3 | IRF1/NM002198.2 | S1PR4/NM003775.3 |
| BCL7C/NM004765.3 | IRF2/NM002199.3 | S1PR5/NM001166215.1 |
| BCL9/NM004326.3 | IRF2BP1/NM015649.2 | S1PR5/NM030760.4 |
| BCL9L/NM182557.2 | IRF2BP2/NM182972.2 | SAA1/NM199161.4 |
| BCLAF1/NM014739.2 | IRF2BPL/NM024496.3 | SAA2-SAA4/NM001199744.1 |
| BCO1/NM017429.2 | IRF3/NM001571.5 | SAA4/NM006512.3 |
| BCO2/NM031938.5 | IRF4/NM002460.3 | SAAL1/NM138421.2 |
| BCR/NM004327.3 | IRF5/NM001098627.3 | SAC3D1/NM013299.3 |
| BCS1L/NM001079866.1 | IRF5/NM001098630.2 | SACM1L/NM014016.4 |
| BDH1/NM203315.2 | IRF5/NM032643.4 | SACS/NM014363.5 |
| BDH2/NM020139.3 | IRF6/NM006147.3 | SAE1/NM005500.2 |
| BDKRB1/NM000710.3 | IRF7/NM004031.2 | SAFB/NM002967.3 |
| BDKRB2/NM000623.3 | IRF8/NM002163.2 | SAFB2/NM014649.2 |
| BDNF/NM170731.4 | IRF9/NM006084.4 | SALL2/NM005407.2 |
| BDP1/NM018429.2 | IRGC/NM019612.3 | SALL3/NM171999.3 |
| BEAN1/NM001197225.3 | IRGM/NM001145805.1 | SALL4/NM020436.4 |
| BECN1/NM003766.4 | IRGQ/NM001007561.2 | SAMD1/NM138352.1 |
| BECN2/NM001290693.1 | IRS1/NM005544.2 | SAMD10/NM080621.4 |
| BEGAIN/NM001159531.1 | IRS2/NM003749.2 | SAMD11/NM152486.2 |
| BEGAIN/NM020836.3 | IRX1/NM024337.3 | SAMD12/NM001101676.1 |
| BEND3/NM001080450.2 | IRX2/NM033267.4 | SAMD13/NM001134663.1 |
| BEND4/NM207406.3 | IRX4/NM001278632.1 | SAMD13/NM001134664.1 |
| BEND5/NM024603.3 | IRX4/NM001278634.1 | SAMD15/NM001010860.1 |
| BEND6/NM152731.2 | IRX4/NM016358.2 | SAMD3/NM001017373.3 |
| BEST1/NM004183.3 | IRX5/NM005853.5 | SAMD3/NM001258275.2 |
| BEST2/NM017682.2 | IRX6/NM024335.2 | SAMD4A/NM015589.5 |
| BEST4/NM153274.2 | ISCA1/NM030940.3 | SAMD4B/NM018028.3 |
| BET1/NM005868.5 | ISCA2/NM194279.3 | SAMD5/NM001030060.2 |
| BET1L/NM001098787.1 | ISG15/NM005101.3 | SAMD7/NM001304366.1 |
| BFAR/NM016561.2 | ISG20/NM001303233.1 | SAMD8/NM001174156.1 |
| BFSP1/NM001195.4 | ISG20/NM002201.5 | SAMD9/NM017654.3 |
| BFSP2/NM003571.3 | ISG20L2/NM001303095.1 | SAMD9L/NM152703.3 |
| BGLAP/NM199173.5 | ISL2/NM145805.2 | SAMHD1/NM015474.3 |
| BHLHA15/NM177455.3 | ISLR/NM005545.3 | SAMM50/NM015380.4 |
| BHLHA9/NM001164405.1 | ISLR/NM201526.1 | SAMSN1/NM022136.4 |
| BHLHE22/NM152414.4 | ISLR2/NM001130136.1 | SAP18/NM005870.4 |
| BHLHE23/NM080606.3 | ISLR2/NM001130137.1 | SAP25/NM001168682.1 |
| BHLHE40/NM003670.2 | ISLR2/NM001130138.1 | SAP30/NM003864.3 |
| BHLHE41/NM030762.2 | ISLR2/NM020851.2 | SAP30BP/NM013260.7 |
| BHMG1/NM001310124.1 | ISM1/NM080826.1 | SAP30L/NM024632.5 |
| BHMT/NM001713.2 | ISM2/NM199296.2 | SAPCD1/NM001039651.1 |
| BHMT2/NM017614.4 | ISOC1/NM016048.2 | SAPCD2/NM178448.3 |
| BICC1/NM001080512.2 | ISOC2/NM001136201.1 | SAR1A/NM001142648.1 |
| BICD1/NM001714.2 | ISPD/NM001101426.3 | SARAF/NM016127.5 |
| BICD2/NM015250.3 | IST1/NM001270975.1 | SARDH/NM001134707.1 |
| BICDL1/NM207311.2 | ISX/NM001303508.1 | SARDH/NM007101.3 |
| BICDL2/NM001103175.1 | ISY1-RAB43/NM001204890.1 | SARM1/NM015077.3 |
| BID/NM197966.2 | ISYNA1/NM016368.4 | SARNP/NM033082.3 |
| BIK/NM001197.4 | ITCH/NM031483.6 | SARS/NM006513.3 |
| BIN1/NM139351.2 | ITFG1/NM030790.4 | SARS2/NM017827.3 |
| BIN2/NM016293.3 | ITFG2/NM018463.3 | SART1/NM005146.4 |
| BIN3/NM018688.4 | ITGA1/NM181501.1 | SASH1/NM015278.3 |
| BIRC2/NM001166.4 | ITGA10/NM003637.4 | SASS6/NM194292.2 |
| BIRC2/NM001256163.1 | ITGA11/NM001004439.1 | SAT2/NM133491.4 |
| BIRC3/NM182962.2 | ITGA2/NM002203.3 | SATB1/NM001131010.3 |
| BIRC5/NM001168.2 | ITGA2B/NM000419.4 | SATB1/NM002971.5 |
| BIRC6/NM016252.3 | ITGA3/NM002204.3 | SATB2/NM001172509.1 |
| BIRC7/NM139317.2 | ITGA4/NM000885.5 | SATB2/NM001172517.1 |
| BIRC8/NM033341.4 | ITGA5/NM002205.4 | SATB2/NM015265.3 |
| BIVM-ERCC5/NM001204425.1 | ITGA6/NM000210.3 | SAV1/NM021818.3 |
| BIVM/NM017693.3 | ITGA7/NM002206.2 | SAXO1/NM153707.3 |
| BLCAP/NM001167820.1 | ITGA8/NM003638.2 | SAXO2/NM001008226.1 |
| BLCAP/NM001167822.2 | ITGA9/NM002207.2 | SAYSD1/NM018322.2 |
| BLCAP/NM001167823.1 | ITGAD/NM005353.2 | SBDS/NM016038.2 |
| BLCAP/NM001317074.1 | ITGAE/NM002208.4 | SBF1/NM002972.3 |
| BLCAP/NM001317075.1 | ITGAL/NM002209.2 | SBF2/NM030962.3 |
| BLID/NM001001786.2 | ITGAM/NM000632.3 | SBK1/NM001024401.2 |
| BLK/NM001715.2 | ITGAV/NM002210.4 | SBK2/NM001101401.2 |
| BLM/NM000057.3 | ITGAX/NM000887.4 | SBK3/NM001199824.2 |
| BLMH/NM000386.3 | ITGB1/NM002211.3 | SBNO1/NM018183.3 |
| BLNK/NM013314.3 | ITGB1/NM133376.2 | SBNO2/NM014963.2 |
| BLOC1S1/NM001487.3 | ITGB1BP1/NM001319066.1 | SBSPON/NM153225.3 |
| BLOC1S2/NM173809.4 | ITGB1BP1/NM001319068.1 | SC5D/NM001024956.2 |
| BLOC1S3/NM212550.4 | ITGB1BP1/NM004763.4 | SC5D/NM006918.4 |
| BLOC1S4/NM018366.2 | ITGB2/NM000211.4 | SCAF1/NM021228.2 |
| BLOC1S5/NM201280.2 | ITGB2/NM001127491.2 | SCAF4/NM020706.2 |
| BLOC1S6/NM012388.3 | ITGB3/NM000212.2 | SCAF8/NM001286199.1 |
| BLVRA/NM000712.3 | ITGB4/NM000213.4 | SCAI/NM001144877.2 |
| BLVRB/NM000713.2 | ITGB5/NM002213.4 | SCAMP1/NM004866.5 |
| BLZF1/NM001320973.1 | ITGB6/NM000888.4 | SCAMP2/NM005697.4 |
| BMF/NM001003940.1 | ITGB7/NM000889.2 | SCAMP3/NM005698.3 |
| BMF/NM033503.3 | ITGB8/NM002214.2 | SCAMP4/NM079834.2 |
| BMI1/NM005180.8 | ITGBL1/NM004791.2 | SCAMP5/NM138967.3 |
| BMP1/NM006129.4 | ITIH1/NM002215.3 | SCAND1/NM016558.3 |
| BMP10/NM014482.1 | ITIH2/NM002216.2 | SCAP/NM012235.3 |
| BMP2/NM001200.3 | ITIH3/NM002217.3 | SCAPER/NM020843.2 |
| BMP2K/NM198892.1 | ITIH4/NM002218.4 | SCARA3/NM016240.2 |
| BMP3/NM001201.2 | ITIH5/NM030569.6 | SCARA5/NM173833.5 |
| BMP4/NM001202.4 | ITK/NM005546.3 | SCARB1/NM005505.4 |
| BMP5/NM021073.2 | ITLN1/NM017625.2 | SCARB2/NM005506.3 |
| BMP6/NM001718.4 | ITLN2/NM080878.2 | SCARF1/NM003693.3 |
| BMP7/NM001719.2 | ITM2B/NM021999.4 | SCARF2/NM153334.6 |
| BMP8A/NM181809.3 | ITM2C/NM001287241.1 | SCCPDH/NM016002.2 |
| BMP8B/NM001720.3 | ITM2C/NM030926.5 | SCD/NM005063.4 |
| BMPER/NM133468.4 | ITPA/NM033453.3 | SCD5/NM001037582.2 |
| BMPR1A/NM004329.2 | ITPK1/NM014216.4 | SCFD1/NM016106.3 |
| BMPR1B/NM001256793.1 | ITPKA/NM002220.2 | SCG2/NM003469.4 |
| BMPR2/NM001204.6 | ITPKB/NM002221.3 | SCG3/NM013243.3 |
| BMS1/NM014753.3 | ITPR1/NM001099952.2 | SCG5/NM001144757.1 |
| BMT2/NM152556.2 | ITPR2/NM002223.3 | SCGB1A1/NM003357.4 |
| BNC1/NM001717.3 | ITPR3/NM002224.3 | SCGB1C1/NM145651.2 |
| BNC2/NM017637.5 | ITPRIP/NM001272012.1 | SCGB1C2/NM001097610.2 |
| BNIP1/NM001205.2 | ITPRIP/NM001272013.1 | SCGB1D1/NM006552.1 |
| BNIP2/NM004330.3 | ITPRIP/NM033397.3 | SCGB1D2/NM006551.3 |
| BNIP3/NM004052.3 | ITPRIPL1/NM001163523.1 | SCGB1D4/NM206998.1 |
| BNIP3L/NM004331.2 | ITPRIPL1/NM001163524.1 | SCGB2A1/NM002407.2 |
| BNIPL/NM138278.3 | ITPRIPL2/NM001034841.3 | SCGB2A2/NM002411.3 |
| BOC/NM033254.3 | ITSN1/NM003024.2 | SCGB2B2/NM001025591.3 |
| BOD1L1/NM148894.2 | ITSN2/NM006277.2 | SCGB3A1/NM052863.2 |
| BOD1L2/NM001257964.1 | IVD/NM002225.3 | SCGB3A2/NM054023.4 |
| BOK/NM032515.4 | IVL/NM005547.2 | SCGN/NM006998.3 |
| BOLA1/NM016074.4 | IVNS1ABP/NM006469.4 | SCHIP1/NM001197109.1 |
| BOLA2-SMG1P6/NM001320623.1 | IWS1/NM017969.2 | SCIMP/NM207103.3 |
| BOLA2B/NM001039182.2 | IYD/NM203395.2 | SCIN/NM001112706.2 |
| BOLA3/NM212552.2 | IZUMO1/NM182575.2 | SCLT1/NM144643.3 |
| BOLL/NM197970.2 | IZUMO1R/NM001199206.1 | SCLY/NM016510.5 |
| BOP1/NM015201.4 | IZUMO2/NM152358.2 | SCMH1/NM012236.3 |
| BORA/NM024808.3 | IZUMO4/NM001031735.2 | SCML4/NM198081.4 |
| BORCS5/NM058169.4 | JADE1/NM001287440.1 | SCN10A/NM006514.3 |
| BORCS6/NM017622.2 | JADE1/NM001287442.1 | SCN11A/NM001287223.1 |
| BORCS7/NM144591.3 | JADE1/NM001287443.1 | SCN11A/NM014139.2 |
| BORCS8-MEF2B/NM005919.3 | JADE1/NM199320.3 | SCN1A/NM006920.4 |
| BPGM/NM001724.4 | JADE2/NM015288.5 | SCN1B/NM001037.4 |
| BPHL/NM004332.3 | JAG1/NM000214.2 | SCN2A/NM001040142.1 |
| BPI/NM001725.2 | JAG2/NM002226.4 | SCN2B/NM004588.4 |
| BPIFA2/NM001319164.1 | JAGN1/NM032492.3 | SCN3A/NM006922.3 |
| BPIFA2/NM080574.3 | JAK1/NM002227.3 | SCN3B/NM018400.3 |
| BPIFB1/NM033197.2 | JAK2/NM001322195.1 | SCN4A/NM000334.4 |
| BPIFB2/NM025227.2 | JAK2/NM001322196.1 | SCN4B/NM174934.3 |
| BPIFB3/NM182658.2 | JAK2/NM004972.3 | SCN5A/NM001099404.1 |
| BPIFB4/NM182519.2 | JAK3/NM000215.3 | SCN7A/NM002976.3 |
| BPIFB6/NM174897.2 | JAKMIP1/NM001306133.1 | SCN8A/NM014191.3 |
| BPNT1/NM006085.5 | JAKMIP1/NM144720.3 | SCN9A/NM002977.3 |
| BRAF/NM004333.4 | JAKMIP2/NM014790.4 | SCNM1/NM024041.3 |
| BRAP/NM006768.4 | JAKMIP3/NM001105521.3 | SCNN1A/NM001038.5 |
| BRAT1/NM152743.3 | JAM2/NM021219.3 | SCNN1B/NM000336.2 |
| BRCA1/NM007300.3 | JAM3/NM032801.4 | SCNN1D/NM001130413.3 |
| BRCA2/NM000059.3 | JAML/NM153206.2 | SCNN1G/NM001039.3 |
| BRD1/NM001304809.1 | JARID2/NM004973.3 | SCO1/NM004589.3 |
| BRD2/NM001113182.2 | JAZF1/NM175061.3 | SCO2/NM001169109.1 |
| BRD2/NM005104.3 | JCHAIN/NM144646.3 | SCO2/NM001169110.1 |
| BRD3/NM007371.3 | JDP2/NM001135047.1 | SCO2/NM001169111.1 |
| BRD4/NM058243.2 | JDP2/NM001135048.1 | SCO2/NM005138.2 |
| BRD7/NM013263.4 | JDP2/NM130469.3 | SCOC/NM001153446.1 |
| BRD8/NM139199.1 | JMJD1C/NM032776.2 | SCOC/NM001153585.1 |
| BRD9/NM023924.4 | JMJD4/NM023007.2 | SCOC/NM032547.2 |
| BRDT/NM001726.4 | JMJD6/NM001081461.1 | SCP2/NM002979.4 |
| BRE/NM199191.2 | JMJD8/NM001005920.2 | SCP2D1/NM178483.2 |
| BRF1/NM001519.3 | JMY/NM152405.4 | SCPEP1/NM021626.2 |
| BRF2/NM018310.3 | JOSD1/NM014876.5 | SCRG1/NM007281.2 |
| BRI3/NM015379.4 | JOSD2/NM001270639.1 | SCRIB/NM182706.4 |
| BRI3BP/NM080626.5 | JOSD2/NM138334.3 | SCRN1/NM001145513.1 |
| BRICD5/NM182563.3 | JPH1/NM020647.3 | SCRN1/NM014766.4 |
| BRINP2/NM021165.3 | JPH2/NM020433.4 | SCRN2/NM138355.3 |
| BRINP3/NM199051.2 | JPH3/NM020655.3 | SCRN3/NM024583.4 |
| BRIP1/NM032043.2 | JPH4/NM032452.2 | SCRT1/NM031309.5 |
| BRIX1/NM018321.3 | JRK/NM003724.3 | SCRT2/NM033129.3 |
| BRK1/NM018462.4 | JRKL/NM001261833.1 | SCT/NM021920.3 |
| BRMS1/NM015399.3 | JSRP1/NM144616.3 | SCTR/NM002980.2 |
| BRMS1L/NM032352.3 | JTB/NM006694.3 | SCUBE1/NM173050.3 |
| BROX/NM144695.3 | JUN/NM002228.3 | SCUBE2/NM020974.2 |
| BRPF1/NM004634.2 | JUNB/NM002229.2 | SCUBE3/NM152753.3 |
| BRPF3/NM015695.2 | JUND/NM005354.5 | SCX/NM001080514.2 |
| BRSK1/NM032430.1 | JUP/NM002230.2 | SCYL1/NM020680.3 |
| BRSK2/NM003957.3 | KAAG1/NM181337.3 | SCYL2/NM017988.5 |
| BRWD1/NM018963.4 | KALRN/NM001024660.4 | SCYL3/NM181093.3 |
| BSCL2/NM032667.6 | KANK1/NM001256876.1 | SDAD1/NM018115.3 |
| BSDC1/NM018045.7 | KANK1/NM015158.3 | SDC1/NM001006946.1 |
| BSG/NM001728.3 | KANK2/NM001136191.2 | SDC1/NM002997.4 |
| BSN/NM003458.3 | KANK3/NM198471.2 | SDC2/NM002998.3 |
| BSND/NM057176.2 | KANK4/NM181712.4 | SDC3/NM014654.3 |
| BSPH1/NM001128326.2 | KANSL1/NM001193465.1 | SDC4/NM002999.3 |
| BSPRY/NM017688.2 | KANSL1L/NM152519.3 | SDCBP/NM001007067.1 |
| BST1/NM004334.2 | KANSL2/NM017822.3 | SDCBP2/NM015685.5 |
| BST2/NM004335.3 | KARS/NM005548.2 | SDCCAG3/NM006643.3 |
| BSX/NM001098169.1 | KAT14/NM020536.4 | SDCCAG8/NM006642.3 |
| BTBD1/NM025238.3 | KAT2A/NM021078.2 | SDE2/NM152608.3 |
| BTBD10/NM032320.6 | KAT2B/NM003884.4 | SDF2/NM006923.3 |
| BTBD11/NM001018072.1 | KAT5/NM182710.2 | SDF2L1/NM022044.2 |
| BTBD17/NM001080466.1 | KAT6A/NM006766.4 | SDF4/NM016547.2 |
| BTBD18/NM001145101.1 | KAT6B/NM012330.3 | SDHA/NM004168.3 |
| BTBD19/NM001136537.1 | KAT7/NM007067.4 | SDHAF1/NM001042631.2 |
| BTBD2/NM017797.3 | KAT8/NM032188.2 | SDHAF2/NM017841.2 |
| BTBD3/NM001282550.1 | KATNA1/NM007044.3 | SDHAF3/NM020186.2 |
| BTBD3/NM001282551.1 | KATNAL1/NM001014380.2 | SDHAF4/NM145267.2 |
| BTBD6/NM033271.2 | KATNAL1/NM032116.4 | SDHB/NM003000.2 |
| BTBD7/NM001002860.3 | KATNB1/NM005886.2 | SDHC/NM003001.3 |
| BTBD8/NM183242.3 | KATNBL1/NM024713.2 | SDHD/NM003002.3 |
| BTBD9/NM052893.1 | KAZALD1/NM030929.4 | SDK1/NM152744.3 |
| BTC/NM001729.3 | KAZN/NM201628.2 | SDK2/NM001144952.1 |
| BTD/NM000060.3 | KBTBD11/NM014867.2 | SDPR/NM004657.5 |
| BTF3/NM001207.4 | KBTBD12/NM207335.2 | SDR16C5/NM138969.3 |
| BTF3L4/NM152265.4 | KBTBD13/NM001101362.2 | SDR39U1/NM020195.2 |
| BTG1/NM001731.2 | KBTBD2/NM015483.2 | SDR42E1/NM145168.2 |
| BTG2/NM006763.2 | KBTBD3/NM198439.2 | SDR9C7/NM148897.2 |
| BTG3/NM006806.4 | KBTBD4/NM016506.6 | SDS/NM006843.2 |
| BTG4/NM017589.3 | KBTBD6/NM152903.4 | SDSL/NM001304993.1 |
| BTLA/NM181780.3 | KBTBD7/NM032138.4 | SEBOX/NM001080837.3 |
| BTN1A1/NM001732.2 | KBTBD8/NM032505.2 | SEC11A/NM014300.3 |
| BTN2A1/NM007049.4 | KCMF1/NM020122.4 | SEC14L1/NM001039573.2 |
| BTN2A2/NM001197237.1 | KCNA1/NM000217.2 | SEC14L1/NM001204408.1 |
| BTN2A2/NM006995.4 | KCNA10/NM005549.2 | SEC14L2/NM012429.4 |
| BTN3A1/NM007048.5 | KCNA2/NM004974.3 | SEC14L3/NM174975.4 |
| BTN3A2/NM001197246.2 | KCNA3/NM002232.4 | SEC14L5/NM014692.1 |
| BTN3A2/NM007047.4 | KCNA4/NM002233.3 | SEC14L6/NM001193336.2 |
| BTN3A3/NM006994.4 | KCNA5/NM002234.3 | SEC16A/NM014866.1 |
| BTNL10/NM001287262.1 | KCNA6/NM002235.3 | SEC16B/NM033127.2 |
| BTNL2/NM001304561.1 | KCNA7/NM031886.2 | SEC22A/NM012430.4 |
| BTNL3/NM197975.2 | KCNAB1/NM172160.2 | SEC22B/NM004892.5 |
| BTNL8/NM001040462.2 | KCNAB2/NM001199860.1 | SEC22C/NM032970.3 |
| BTNL9/NM152547.4 | KCNAB2/NM001199861.1 | SEC23A/NM006364.3 |
| BTRC/NM033637.3 | KCNAB2/NM003636.3 | SEC23B/NM001172745.1 |
| BUB1/NM004336.4 | KCNAB3/NM004732.3 | SEC23B/NM032986.3 |
| BUB1B-PAK6/NM001128629.2 | KCNB1/NM004975.3 | SEC23IP/NM007190.3 |
| BUB1B/NM001211.5 | KCNB2/NM004770.2 | SEC24A/NM021982.2 |
| BUB3/NM004725.3 | KCNC1/NM001112741.1 | SEC24B/NM001042734.3 |
| BUD13/NM032725.3 | KCNC2/NM139137.3 | SEC24C/NM198597.2 |
| BUD31/NM003910.3 | KCNC3/NM004977.2 | SEC24D/NM014822.3 |
| BVES/NM001199563.1 | KCNC4/NM001039574.2 | SEC31A/NM001077207.3 |
| BVES/NM007073.4 | KCND2/NM012281.2 | SEC31B/NM015490.3 |
| BVES/NM147147.3 | KCND3/NM004980.4 | SEC61A1/NM013336.3 |
| BYSL/NM004053.3 | KCNE1/NM000219.5 | SEC61A2/NM018144.3 |
| BZW1/NM001207068.1 | KCNE1/NM001127668.3 | SEC61B/NM006808.2 |
| BZW2/NM014038.2 | KCNE1/NM001127670.3 | SEC61G/NM014302.3 |
| C10orf10/NM007021.3 | KCNE1/NM001270402.2 | SEC62/NM003262.3 |
| C10orf105/NM001164375.2 | KCNE1/NM001270403.2 | SEC63/NM007214.4 |
| C10orf105/NM001168390.1 | KCNE1/NM001270405.2 | SECISBP2/NM024077.4 |
| C10orf107/NM173554.2 | KCNE2/NM172201.1 | SECISBP2L/NM014701.3 |
| C10orf11/NM032024.4 | KCNE3/NM005472.4 | SECTM1/NM003004.2 |
| C10orf111/NM153244.1 | KCNE4/NM080671.3 | SEH1L/NM001013437.1 |
| C10orf113/NM001010896.2 | KCNF1/NM002236.4 | SEL1L3/NM015187.4 |
| C10orf12/NM015652.2 | KCNG1/NM002237.3 | SELE/NM000450.2 |
| C10orf120/NM001010912.2 | KCNG2/NM012283.1 | SELENBP1/NM003944.3 |
| C10orf126/NM001278522.1 | KCNG3/NM133329.5 | SELK/NM021237.3 |
| C10orf128/NM001010863.2 | KCNG4/NM172347.2 | SELM/NM080430.2 |
| C10orf131/NM001130446.2 | KCNH2/NM000238.3 | SELO/NM031454.1 |
| C10orf142/NM001258000.1 | KCNH3/NM012284.2 | SELP/NM003005.3 |
| C10orf2/NM021830.4 | KCNH4/NM012285.2 | SELPLG/NM003006.4 |
| C10orf25/NM001039380.3 | KCNH8/NM144633.2 | SELT/NM016275.3 |
| C10orf35/NM145306.2 | KCNIP1/NM014592.3 | SEMA3A/NM006080.2 |
| C10orf54/NM022153.1 | KCNIP2/NM173191.2 | SEMA3B/NM001290060.1 |
| C10orf55/NM001001791.2 | KCNIP3/NM013434.4 | SEMA3B/NM004636.3 |
| C10orf62/NM001009997.2 | KCNIP4/NM147183.3 | SEMA3C/NM006379.3 |
| C10orf76/NM024541.2 | KCNJ1/NM000220.4 | SEMA3D/NM152754.2 |
| C10orf82/NM144661.2 | KCNJ10/NM002241.4 | SEMA3F/NM004186.4 |
| C10orf88/NM024942.3 | KCNJ11/NM000525.3 | SEMA3G/NM020163.1 |
| C10orf91/NM173541.2 | KCNJ12/NM021012.4 | SEMA4A/NM001193300.1 |
| C10orf95/NM024886.2 | KCNJ13/NM002242.4 | SEMA4A/NM001193301.1 |
| C10orf99/NM207373.2 | KCNJ14/NM013348.3 | SEMA4B/NM020210.3 |
| C11orf1/NM022761.2 | KCNJ15/NM001276438.1 | SEMA4C/NM017789.4 |
| C11orf21/NM001142946.1 | KCNJ15/NM002243.4 | SEMA4D/NM006378.3 |
| C11orf24/NM022338.3 | KCNJ15/NM170736.2 | SEMA4F/NM004263.4 |
| C11orf40/NM144663.1 | KCNJ15/NM170737.2 | SEMA4G/NM017893.3 |
| C11orf42/NM173525.2 | KCNJ16/NM001291624.1 | SEMA5A/NM003966.2 |
| C11orf44/NM001271983.1 | KCNJ18/NM001194958.2 | SEMA5B/NM001031702.3 |
| C11orf45/NM001256088.1 | KCNJ2/NM000891.2 | SEMA5B/NM001256346.1 |
| C11orf45/NM145013.2 | KCNJ3/NM002239.3 | SEMA6A/NM020796.4 |
| C11orf49/NM024113.4 | KCNJ4/NM004981.1 | SEMA6B/NM032108.3 |
| C11orf52/NM080659.2 | KCNJ4/NM152868.2 | SEMA6C/NM030913.4 |
| C11orf54/NM001286068.1 | KCNJ6/NM002240.4 | SEMA6D/NM153618.1 |
| C11orf57/NM001082969.1 | KCNJ8/NM004982.3 | SEMA7A/NM003612.4 |
| C11orf63/NM024806.3 | KCNJ9/NM004983.2 | SEMG1/NM003007.4 |
| C11orf65/NM152587.3 | KCNK1/NM002245.3 | SEMG2/NM003008.2 |
| C11orf68/NM031450.3 | KCNK10/NM138317.2 | SENP1/NM001267594.1 |
| C11orf71/NM019021.3 | KCNK12/NM022055.1 | SENP1/NM001267595.1 |
| C11orf73/NM016401.3 | KCNK13/NM022054.3 | SENP2/NM021627.2 |
| C11orf74/NM001276724.1 | KCNK15/NM022358.3 | SENP3/NM015670.5 |
| C11orf74/NM138787.3 | KCNK16/NM001135106.1 | SENP5/NM152699.4 |
| C11orf80/NM001302084.1 | KCNK17/NM031460.3 | SENP6/NM001100409.2 |
| C11orf84/NM138471.2 | KCNK18/NM181840.1 | SENP7/NM020654.4 |
| C11orf86/NM001136485.1 | KCNK2/NM014217.3 | SENP8/NM001166340.1 |
| C11orf87/NM207645.3 | KCNK3/NM002246.2 | SENP8/NM001172109.1 |
| C11orf88/NM207430.2 | KCNK4/NM001317090.1 | SENP8/NM001172110.1 |
| C11orf91/NM001166692.1 | KCNK4/NM033310.2 | SENP8/NM145204.3 |
| C11orf94/NM001080446.2 | KCNK5/NM003740.3 | SEP15/NM004261.3 |
| C11orf95/NM001144936.1 | KCNK6/NM004823.1 | SEPHS1/NM012247.4 |
| C11orf96/NM001145033.1 | KCNK7/NM033347.1 | SEPHS2/NM012248.3 |
| C11orf97/NM001190462.1 | KCNK9/NM001282534.1 | SEPN1/NM020451.2 |
| C11orf98/NM001286086.1 | KCNMA1/NM002247.3 | SEPP1/NM001085486.1 |
| C12orf10/NM021640.3 | KCNMB1/NM004137.3 | SEPSECS/NM016955.3 |
| C12orf29/NM001009894.2 | KCNMB2/NM181361.2 | SEPT1/NM052838.4 |
| C12orf4/NM020374.3 | KCNMB3/NM014407.3 | SEPT10/NM178584.3 |
| C12orf40/NM001031748.3 | KCNMB4/NM014505.5 | SEPT11/NM018243.3 |
| C12orf42/NM001099336.2 | KCNN1/NM002248.4 | SEPT12/NM144605.4 |
| C12orf43/NM022895.2 | KCNN2/NM021614.3 | SEPT14/NM207366.2 |
| C12orf45/NM152318.2 | KCNN3/NM170782.2 | SEPT2/NM001321030.1 |
| C12orf49/NM024738.2 | KCNN4/NM002250.2 | SEPT2/NM004404.4 |
| C12orf54/NM152319.3 | KCNQ1/NM000218.2 | SEPT2/NM006155.2 |
| C12orf56/NM001170633.1 | KCNQ2/NM172107.3 | SEPT3/NM145733.2 |
| C12orf57/NM001301834.1 | KCNQ3/NM004519.3 | SEPT4/NM004574.4 |
| C12orf57/NM138425.3 | KCNQ4/NM004700.3 | SEPT5/NM002688.5 |
| C12orf60/NM175874.3 | KCNQ5/NM019842.3 | SEPT7/NM001788.5 |
| C12orf65/NM001143905.2 | KCNRG/NM173605.1 | SEPT8/NM001098811.1 |
| C12orf65/NM001194995.1 | KCNS1/NM002251.4 | SEPT9/NM001113491.1 |
| C12orf65/NM152269.4 | KCNS2/NM020697.3 | SEPW1/NM003009.2 |
| C12orf66/NM152440.4 | KCNS3/NM001282428.1 | SERAC1/NM032861.3 |
| C12orf71/NM001080406.1 | KCNS3/NM002252.4 | SERBP1/NM015640.3 |
| C12orf73/NM001135570.1 | KCNT1/NM020822.2 | SERF1A/NM022968.1 |
| C12orf74/NM001037671.3 | KCNT2/NM198503.3 | SERF1B/NM001178087.1 |
| C12orf75/NM001145199.1 | KCNV2/NM133497.3 | SERF2/NM001018108.3 |
| C12orf76/NM207435.2 | KCP/NM001135914.1 | SERF2/NM001199877.1 |
| C12orf77/NM001101339.2 | KCTD1/NM001142730.2 | SERHL2/NM014509.4 |
| C12orf80/NM001242696.1 | KCTD10/NM031954.4 | SERINC1/NM020755.3 |
| C14orf1/NM007176.3 | KCTD11/NM001002914.2 | SERINC2/NM178865.4 |
| C14orf105/NM018168.3 | KCTD12/NM138444.3 | SERINC3/NM198941.2 |
| C14orf119/NM017924.3 | KCTD13/NM178863.4 | SERINC4/NM001258031.1 |
| C14orf132/NM001282464.1 | KCTD14/NM023930.3 | SERINC5/NM001174072.2 |
| C14orf142/NM032490.4 | KCTD15/NM001129995.1 | SERP1/NM014445.3 |
| C14orf159/NM001102367.2 | KCTD17/NM024681.3 | SERP2/NM001010897.1 |
| C14orf159/NM001286471.1 | KCTD18/NM152387.3 | SERPINA1/NM000295.4 |
| C14orf159/NM024952.7 | KCTD19/NM001100915.1 | SERPINA1/NM001127701.1 |
| C14orf166/NM016039.2 | KCTD2/NM015353.2 | SERPINA11/NM001080451.1 |
| C14orf169/NM024644.4 | KCTD20/NM173562.4 | SERPINA12/NM001304461.1 |
| C14orf177/NM182560.2 | KCTD21/NM001029859.2 | SERPINA12/NM173850.3 |
| C14orf180/NM001008404.2 | KCTD3/NM016121.4 | SERPINA3/NM001085.4 |
| C14orf180/NM001286400.1 | KCTD4/NM198404.2 | SERPINA4/NM006215.3 |
| C14orf28/NM001017923.1 | KCTD5/NM018992.3 | SERPINA5/NM000624.5 |
| C14orf37/NM001001872.3 | KCTD6/NM001128214.1 | SERPINA6/NM001756.3 |
| C14orf39/NM174978.2 | KCTD6/NM153331.3 | SERPINB1/NM030666.3 |
| C14orf79/NM174891.3 | KCTD7/NM153033.4 | SERPINB10/NM005024.2 |
| C14orf80/NM001134875.1 | KCTD8/NM198353.2 | SERPINB11/NM080475.3 |
| C14orf93/NM001130706.2 | KCTD9/NM017634.3 | SERPINB12/NM001307928.1 |
| C15orf32/NM153040.2 | KDELC1/NM024089.2 | SERPINB13/NM012397.3 |
| C15orf38-AP3S2/NM001199058.1 | KDELC2/NM153705.4 | SERPINB2/NM002575.2 |
| C15orf39/NM015492.4 | KDELR1/NM006801.2 | SERPINB3/NM006919.2 |
| C15orf40/NM144597.2 | KDELR2/NM006854.3 | SERPINB5/NM002639.4 |
| C15orf41/NM001130010.2 | KDELR3/NM006855.3 | SERPINB6/NM001271825.1 |
| C15orf48/NM197955.2 | KDF1/NM152365.2 | SERPINB6/NM001297699.1 |
| C15orf52/NM207380.2 | KDM1A/NM015013.3 | SERPINB7/NM001040147.2 |
| C15orf53/NM207444.2 | KDM2A/NM012308.2 | SERPINB7/NM001261830.1 |
| C15orf54/NM001302797.1 | KDM3A/NM001146688.1 | SERPINB7/NM003784.3 |
| C15orf56/NM001039905.2 | KDM3A/NM018433.5 | SERPINB8/NM002640.3 |
| C15orf57/NM052849.3 | KDM3B/NM016604.3 | SERPINB9/NM004155.5 |
| C15orf59/NM001039614.2 | KDM4A/NM014663.2 | SERPINC1/NM000488.3 |
| C15orf61/NM001143936.1 | KDM4B/NM015015.2 | SERPIND1/NM000185.3 |
| C15orf62/NM001130448.2 | KDM4C/NM015061.3 | SERPINE1/NM000602.4 |
| C15orf65/NM001198784.1 | KDM4D/NM018039.2 | SERPINE2/NM006216.3 |
| C16orf13/NM032366.4 | KDM4E/NM001161630.1 | SERPINE3/NM001101320.1 |
| C16orf45/NM033201.2 | KDM5A/NM001042603.2 | SERPINF1/NM002615.5 |
| C16orf46/NM152337.2 | KDM5B/NM006618.4 | SERPINF2/NM000934.3 |
| C16orf47/NM207385.1 | KDM6B/NM001080424.1 | SERPINF2/NM001165920.1 |
| C16orf52/NM001164579.1 | KDM7A/NM030647.1 | SERPING1/NM000062.2 |
| C16orf54/NM175900.3 | KDM8/NM024773.2 | SERPING1/NM001032295.1 |
| C16orf58/NM022744.3 | KDR/NM002253.2 | SERPINH1/NM001235.3 |
| C16orf59/NM025108.2 | KDSR/NM002035.2 | SERPINI1/NM001122752.1 |
| C16orf62/NM020314.5 | KEAP1/NM012289.3 | SERPINI1/NM005025.4 |
| C16orf70/NM001320540.1 | KEAP1/NM203500.1 | SERPINI2/NM006217.4 |
| C16orf70/NM025187.4 | KEL/NM000420.2 | SERTAD1/NM013376.3 |
| C16orf71/NM139170.2 | KERA/NM007035.3 | SERTAD2/NM014755.2 |
| C16orf72/NM014117.2 | KHDC1/NM030568.4 | SERTAD3/NM013368.3 |
| C16orf74/NM206967.2 | KHDC1L/NM001126063.2 | SERTAD3/NM203344.2 |
| C16orf78/NM144602.3 | KHDC3L/NM001017361.2 | SERTAD4/NM019605.3 |
| C16orf82/NM001145545.1 | KHDRBS1/NM006559.2 | SERTM1/NM203451.2 |
| C16orf86/NM001012984.2 | KHDRBS2/NM152688.3 | SESN1/NM014454.2 |
| C16orf87/NM001001436.2 | KHDRBS3/NM006558.2 | SESN2/NM031459.4 |
| C16orf89/NM152459.4 | KHNYN/NM001290257.1 | SESN3/NM144665.3 |
| C16orf90/NM001080524.1 | KHSRP/NM003685.2 | SESTD1/NM178123.4 |
| C16orf91/NM001272051.1 | KIAA0040/NM001162893.1 | SET/NM003011.3 |
| C16orf92/NM001109660.1 | KIAA0040/NM001319231.1 | SETBP1/NM015559.2 |
| C16orf95/NM001195124.1 | KIAA0100/NM014680.3 | SETD1A/NM014712.2 |
| C16orf96/NM001145011.1 | KIAA0101/NM014736.5 | SETD1B/NM015048.1 |
| C16orf97/NM001242473.1 | KIAA0141/NM001142603.2 | SETD2/NM014159.6 |
| C17orf100/NM001105520.1 | KIAA0196/NM014846.3 | SETD3/NM032233.2 |
| C17orf102/NM207454.2 | KIAA0226L/NM001286761.1 | SETD4/NM017438.4 |
| C17orf105/NM001136483.1 | KIAA0226L/NM025113.3 | SETD5/NM001080517.2 |
| C17orf107/NM001145536.1 | KIAA0232/NM001100590.1 | SETD6/NM024860.3 |
| C17orf112/NM001243552.1 | KIAA0319/NM001168375.1 | SETD7/NM030648.3 |
| C17orf47/NM001038704.2 | KIAA0319/NM014809.3 | SETD9/NM153706.3 |
| C17orf49/NM174893.3 | KIAA0319L/NM024874.4 | SETDB1/NM012432.3 |
| C17orf50/NM145272.3 | KIAA0355/NM014686.3 | SETDB2/NM031915.2 |
| C17orf51/NM001113434.4 | KIAA0391/NM014672.3 | SETMAR/NM006515.3 |
| C17orf53/NM024032.4 | KIAA0408/NM014702.4 | SETSIP/NM001287737.1 |
| C17orf58/NM181655.3 | KIAA0430/NM014647.3 | SETX/NM015046.5 |
| C17orf62/NM001193653.1 | KIAA0513/NM001286565.1 | SEZ6/NM178860.4 |
| C17orf62/NM001193655.1 | KIAA0513/NM014732.3 | SEZ6L/NM021115.4 |
| C17orf67/NM001085430.2 | KIAA0556/NM015202.3 | SEZ6L2/NM201575.3 |
| C17orf74/NM175734.4 | KIAA0586/NM001244189.1 | SF1/NM201995.2 |
| C17orf75/NM022344.3 | KIAA0753/NM014804.2 | SF3A1/NM005877.5 |
| C17orf77/NM001302809.1 | KIAA0754/NM015038.1 | SF3A2/NM007165.4 |
| C17orf77/NM152460.2 | KIAA0895/NM001199708.1 | SF3A3/NM006802.3 |
| C17orf78/NM173625.4 | KIAA0895L/NM001040715.1 | SF3B1/NM012433.3 |
| C17orf80/NM017941.5 | KIAA0907/NM014949.3 | SF3B2/NM006842.2 |
| C17orf82/NM203425.2 | KIAA0922/NM015196.3 | SF3B3/NM012426.4 |
| C17orf89/NM001086521.1 | KIAA0930/NM001009880.1 | SF3B4/NM005850.4 |
| C17orf96/NM001130677.1 | KIAA1024/NM015206.2 | SF3B5/NM031287.2 |
| C17orf98/NM001080465.2 | KIAA1024L/NM001257308.1 | SF3B6/NM016047.3 |
| C17orf99/NM001163075.1 | KIAA1033/NM015275.2 | SFI1/NM001007467.2 |
| C18orf21/NM031446.4 | KIAA1107/NM015237.2 | SFMBT1/NM016329.3 |
| C18orf25/NM145055.3 | KIAA1109/NM015312.3 | SFMBT2/NM001018039.1 |
| C18orf32/NM001035005.3 | KIAA1143/NM020696.3 | SFMBT2/NM001029880.2 |
| C18orf54/NM001288981.1 | KIAA1147/NM001080392.1 | SFN/NM006142.3 |
| C18orf63/NM001174123.1 | KIAA1161/NM020702.4 | SFPQ/NM005066.2 |
| C18orf65/NM001272093.2 | KIAA1191/NM020444.4 | SFR1/NM001002759.1 |
| C18orf8/NM013326.4 | KIAA1211/NM020722.1 | SFRP1/NM003012.4 |
| C19orf12/NM001256047.1 | KIAA1211L/NM207362.2 | SFRP2/NM003013.2 |
| C19orf12/NM031448.4 | KIAA1217/NM019590.4 | SFRP4/NM003014.3 |
| C19orf18/NM152474.4 | KIAA1257/NM020741.2 | SFRP5/NM003015.3 |
| C19orf25/NM152482.2 | KIAA1324/NM020775.4 | SFSWAP/NM004592.3 |
| C19orf33/NM033520.2 | KIAA1324L/NM001291990.1 | SFT2D1/NM145169.2 |
| C19orf35/NM198532.2 | KIAA1328/NM020776.1 | SFT2D2/NM199344.2 |
| C19orf38/NM001136482.1 | KIAA1429/NM015496.4 | SFT2D3/NM032740.3 |
| C19orf44/NM032207.3 | KIAA1456/NM020844.2 | SFTA2/NM205854.2 |
| C19orf45/NM198534.2 | KIAA1462/NM020848.2 | SFTPA2/NM001320813.1 |
| C19orf47/NM001256440.1 | KIAA1468/NM020854.3 | SFTPB/NM000542.3 |
| C19orf52/NM138358.2 | KIAA1522/NM020888.2 | SFTPD/NM003019.4 |
| C19orf53/NM014047.2 | KIAA1524/NM020890.2 | SFXN1/NM022754.6 |
| C19orf54/NM198476.3 | KIAA1549/NM020910.2 | SFXN2/NM178858.4 |
| C19orf57/NM024323.3 | KIAA1549L/NM012194.2 | SFXN3/NM030971.3 |
| C19orf60/NM001100419.1 | KIAA1551/NM018169.3 | SFXN4/NM213649.1 |
| C19orf66/NM018381.3 | KIAA1586/NM020931.3 | SFXN5/NM144579.2 |
| C19orf67/NM001277378.1 | KIAA1614/NM020950.1 | SGCA/NM000023.3 |
| C19orf68/NM199341.3 | KIAA1644/NM001099294.1 | SGCB/NM000232.4 |
| C19orf70/NM205767.2 | KIAA1671/NM001145206.1 | SGCD/NM000337.5 |
| C19orf71/NM001135580.1 | KIAA1683/NM025249.3 | SGCE/NM003919.2 |
| C19orf73/NM018111.2 | KIAA1715/NM030650.2 | SGCG/NM000231.2 |
| C19orf81/NM001195076.1 | KIAA1755/NM001029864.1 | SGCZ/NM139167.3 |
| C19orf84/NM001193623.1 | KIAA1804/NM032435.2 | SGF29/NM138414.2 |
| C1D/NM001190265.1 | KIAA1841/NM001129993.1 | SGK1/NM005627.3 |
| C1GALT1/NM020156.4 | KIAA1958/NM133465.3 | SGK2/NM001199264.1 |
| C1GALT1C1L/NM001101330.2 | KIAA2013/NM138346.2 | SGK2/NM170693.2 |
| C1orf100/NM001012970.2 | KIAA2026/NM001017969.2 | SGK223/NM001080826.2 |
| C1orf101/NM001130957.1 | KIDINS220/NM020738.2 | SGK3/NM001033578.2 |
| C1orf106/NM001142569.2 | KIF11/NM004523.3 | SGK3/NM013257.4 |
| C1orf109/NM017850.2 | KIF13A/NM022113.5 | SGK494/NM001174103.1 |
| C1orf115/NM024709.4 | KIF13B/NM015254.3 | SGMS1/NM147156.3 |
| C1orf116/NM023938.5 | KIF14/NM014875.2 | SGMS2/NM001136257.1 |
| C1orf122/NM198446.2 | KIF15/NM020242.2 | SGMS2/NM001136258.1 |
| C1orf127/NM001170754.1 | KIF16B/NM024704.4 | SGO1/NM001199256.2 |
| C1orf131/NM152379.3 | KIF17/NM020816.3 | SGO2/NM152524.5 |
| C1orf137/NM001013643.1 | KIF18A/NM031217.3 | SGPL1/NM003901.3 |
| C1orf146/NM001012425.1 | KIF18B/NM001265577.1 | SGPP1/NM030791.3 |
| C1orf158/NM152290.3 | KIF19/NM153209.3 | SGPP2/NM152386.3 |
| C1orf159/NM017891.4 | KIF1A/NM004321.6 | SGSH/NM000199.3 |
| C1orf162/NM174896.3 | KIF1B/NM015074.3 | SGSM1/NM001039948.3 |
| C1orf167/NM001010881.1 | KIF1BP/NM015634.3 | SGSM2/NM014853.2 |
| C1orf174/NM207356.2 | KIF1C/NM006612.5 | SGSM3/NM015705.5 |
| C1orf185/NM001136508.1 | KIF20A/NM005733.2 | SGTA/NM003021.3 |
| C1orf186/NM001007544.3 | KIF20B/NM016195.3 | SGTB/NM019072.2 |
| C1orf189/NM001010979.2 | KIF21A/NM017641.3 | SH2B1/NM001145796.1 |
| C1orf194/NM001122961.1 | KIF21B/NM017596.3 | SH2B1/NM001145812.1 |
| C1orf195/NM001278501.1 | KIF22/NM007317.2 | SH2B1/NM015503.2 |
| C1orf198/NM032800.2 | KIF23/NM138555.3 | SH2B2/NM020979.4 |
| C1orf204/NM001134233.1 | KIF24/NM194313.2 | SH2B3/NM005475.2 |
| C1orf21/NM030806.3 | KIF25/NM030615.2 | SH2D1B/NM053282.4 |
| C1orf210/NM001164829.1 | KIF26A/NM015656.1 | SH2D2A/NM003975.3 |
| C1orf210/NM182517.2 | KIF26B/NM018012.3 | SH2D3A/NM005490.2 |
| C1orf216/NM152374.1 | KIF27/NM017576.2 | SH2D3C/NM005489.3 |
| C1orf226/NM001135240.1 | KIF2B/NM032559.4 | SH2D4A/NM001174159.1 |
| C1orf228/NM001145636.1 | KIF2C/NM006845.3 | SH2D4A/NM022071.3 |
| C1orf229/NM207401.2 | KIF3B/NM004798.3 | SH2D4B/NM207372.2 |
| C1orf233/NM001242659.1 | KIF3C/NM002254.6 | SH2D7/NM001101404.1 |
| C1orf234/NM001242521.1 | KIF4B/NM001099293.1 | SH3BGR/NM007341.2 |
| C1orf27/NM017847.5 | KIF5A/NM004984.2 | SH3BGRL2/NM031469.3 |
| C1orf35/NM024319.3 | KIF5B/NM004521.2 | SH3BGRL3/NM031286.3 |
| C1orf43/NM001098616.2 | KIF5C/NM004522.2 | SH3BP1/NM018957.3 |
| C1orf50/NM024097.3 | KIF6/NM145027.4 | SH3BP2/NM001122681.1 |
| C1orf52/NM198077.3 | KIF7/NM198525.2 | SH3BP2/NM003023.4 |
| C1orf53/NM001024594.2 | KIF9/NM001134878.1 | SH3BP4/NM014521.2 |
| C1orf54/NM001301040.1 | KIFAP3/NM014970.3 | SH3BP5/NM004844.4 |
| C1orf54/NM024579.3 | KIFC1/NM002263.3 | SH3BP5L/NM030645.1 |
| C1orf56/NM017860.3 | KIFC2/NM145754.3 | SH3D19/NM001009555.3 |
| C1orf61/NM006365.2 | KIFC3/NM005550.3 | SH3D21/NM001162530.1 |
| C1orf64/NM178840.3 | KIN/NM012311.3 | SH3GL1/NM003025.3 |
| C1orf68/NM001024679.2 | KIR2DL1/NM014218.2 | SH3GL3/NM003027.4 |
| C1orf74/NM152485.3 | KIR2DL3/NM015868.2 | SH3GLB2/NM020145.3 |
| C1orf87/NM152377.2 | KIR2DL4/NM001080770.1 | SH3PXD2A/NM014631.2 |
| C1orf94/NM032884.4 | KIR2DS4/NM001281971.1 | SH3PXD2B/NM001017995.2 |
| C1QA/NM015991.2 | KIR3DL1/NM013289.2 | SH3RF1/NM020870.3 |
| C1QB/NM000491.3 | KIR3DL2/NM006737.3 | SH3RF2/NM152550.3 |
| C1QBP/NM001212.3 | KIR3DL3/NM153443.4 | SH3RF3/NM001099289.2 |
| C1QC/NM172369.3 | KIRREL/NM018240.6 | SH3TC1/NM018986.4 |
| C1QL1/NM006688.4 | KIRREL2/NM199180.3 | SH3TC2/NM024577.3 |
| C1QL2/NM182528.3 | KIRREL3/NM032531.3 | SH3YL1/NM015677.3 |
| C1QL3/NM001010908.1 | KISS1/NM002256.3 | SHANK1/NM016148.3 |
| C1QL4/NM001008223.1 | KISS1R/NM032551.4 | SHANK2/NM012309.4 |
| C1QTNF1/NM030968.3 | KIT/NM000222.2 | SHANK3/NM033517.1 |
| C1QTNF1/NM153372.1 | KITLG/NM000899.4 | SHARPIN/NM030974.3 |
| C1QTNF1/NM198593.3 | KIZ/NM018474.4 | SHB/NM003028.2 |
| C1QTNF2/NM031908.4 | KL/NM004795.3 | SHBG/NM001040.4 |
| C1QTNF3/NM030945.3 | KLB/NM175737.3 | SHC1/NM003029.4 |
| C1QTNF4/NM031909.2 | KLC1/NM005552.4 | SHC2/NM012435.2 |
| C1QTNF5/NM001278431.1 | KLC2/NM001134775.1 | SHC3/NM016848.5 |
| C1QTNF6/NM031910.3 | KLC2/NM001134776.1 | SHC4/NM203349.3 |
| C1QTNF7/NM001135171.1 | KLC2/NM001318734.1 | SHCBP1/NM024745.4 |
| C1QTNF7/NM031911.4 | KLC3/NM177417.2 | SHCBP1L/NM030933.2 |
| C1QTNF8/NM207419.3 | KLC4/NM001289034.1 | SHD/NM020209.3 |
| C1QTNF9/NM178540.4 | KLC4/NM201521.2 | SHE/NM001010846.2 |
| C1QTNF9B-AS1/NM001135816.1 | KLF1/NM006563.3 | SHF/NM138356.2 |
| C1QTNF9B/NM001007537.2 | KLF10/NM005655.3 | SHFM1/NM006304.1 |
| C1R/NM001733.4 | KLF11/NM003597.4 | SHH/NM000193.3 |
| C1RL/NM016546.3 | KLF12/NM007249.4 | SHISA2/NM001007538.1 |
| C1S/NM001734.3 | KLF13/NM015995.3 | SHISA3/NM001080505.1 |
| C2/NM000063.5 | KLF14/NM138693.3 | SHISA4/NM198149.2 |
| C20orf144/NM080825.3 | KLF15/NM014079.3 | SHISA5/NM016479.4 |
| C20orf173/NM001145350.1 | KLF16/NM031918.3 | SHISA6/NM207386.3 |
| C20orf194/NM001009984.2 | KLF17/NM173484.3 | SHISA7/NM001145176.1 |
| C20orf195/NM024059.3 | KLF3/NM016531.5 | SHISA8/NM001207020.1 |
| C20orf196/NM152504.3 | KLF4/NM004235.5 | SHKBP1/NM138392.3 |
| C20orf197/NM001302813.1 | KLF5/NM001730.4 | SHMT1/NM004169.4 |
| C20orf202/NM001009612.2 | KLF6/NM001300.5 | SHMT2/NM005412.5 |
| C20orf203/NM182584.2 | KLF7/NM003709.3 | SHOC2/NM001324336.1 |
| C20orf24/NM018840.4 | KLF9/NM001206.2 | SHOC2/NM007373.3 |
| C20orf27/NM001258430.1 | KLHDC1/NM172193.2 | SHOX2/NM006884.3 |
| C20orf78/NM001242671.2 | KLHDC10/NM014997.3 | SHPK/NM013276.2 |
| C20orf85/NM178456.2 | KLHDC2/NM014315.2 | SHPRH/NM001042683.2 |
| C20orf96/NM153269.2 | KLHDC3/NM057161.3 | SHQ1/NM018130.2 |
| C21orf140/NM001282537.1 | KLHDC4/NM017566.3 | SHROOM1/NM133456.2 |
| C21orf2/NM004928.2 | KLHDC7A/NM152375.2 | SHROOM3/NM020859.3 |
| C21orf33/NM004649.7 | KLHDC7B/NM138433.3 | SHTN1/NM018330.6 |
| C21orf58/NM058180.4 | KLHDC8B/NM173546.2 | SI/NM001041.3 |
| C21orf59/NM021254.2 | KLHDC9/NM152366.4 | SIAE/NM170601.4 |
| C21orf62/NM001162495.2 | KLHL10/NM152467.3 | SIAH1/NM003031.3 |
| C21orf91/NM001100420.1 | KLHL11/NM018143.2 | SIAH2/NM005067.5 |
| C22orf15/NM182520.2 | KLHL12/NM021633.3 | SIAH3/NM198849.2 |
| C22orf23/NM032561.4 | KLHL14/NM020805.2 | SIDT1/NM017699.2 |
| C22orf24/NM001302819.1 | KLHL17/NM198317.2 | SIDT2/NM001040455.1 |
| C22orf31/NM015370.1 | KLHL18/NM025010.4 | SIGIRR/NM001135053.1 |
| C22orf34/NM001289922.2 | KLHL2/NM007246.3 | SIGIRR/NM001135054.1 |
| C22orf39/NM173793.4 | KLHL20/NM014458.3 | SIGLEC1/NM023068.3 |
| C22orf46/NM001142964.1 | KLHL21/NM014851.3 | SIGLEC10/NM033130.4 |
| C2CD2/NM015500.1 | KLHL22/NM032775.3 | SIGLEC11/NM052884.2 |
| C2CD2L/NM014807.4 | KLHL23/NM144711.5 | SIGLEC12/NM053003.3 |
| C2CD3/NM015531.5 | KLHL24/NM017644.3 | SIGLEC14/NM001098612.1 |
| C2CD4A/NM207322.2 | KLHL25/NM022480.3 | SIGLEC15/NM213602.2 |
| C2CD4B/NM001007595.2 | KLHL26/NM018316.1 | SIGLEC5/NM003830.3 |
| C2CD4C/NM001136263.1 | KLHL28/NM017658.4 | SIGLEC6/NM001245.5 |
| C2CD4D/NM001136003.1 | KLHL29/NM052920.1 | SIGLEC7/NM014385.3 |
| C2CD5/NM001286173.1 | KLHL3/NM017415.2 | SIGLEC8/NM014442.2 |
| C2orf15/NM144706.3 | KLHL30/NM198582.3 | SIGLEC9/NM014441.2 |
| C2orf16/NM032266.3 | KLHL31/NM001003760.4 | SIGLECL1/NM173635.2 |
| C2orf27A/NM013310.3 | KLHL32/NM052904.3 | SIGMAR1/NM005866.3 |
| C2orf27B/NM214461.2 | KLHL35/NM001039548.2 | SIK1/NM173354.3 |
| C2orf40/NM032411.2 | KLHL36/NM024731.3 | SIK2/NM015191.2 |
| C2orf42/NM017880.1 | KLHL38/NM001081675.2 | SIK3/NM025164.4 |
| C2orf47/NM024520.2 | KLHL40/NM152393.3 | SIKE1/NM025073.2 |
| C2orf48/NM182626.3 | KLHL41/NM006063.2 | SIL1/NM001037633.1 |
| C2orf49/NM024093.2 | KLHL42/NM020782.1 | SIM1/NM005068.2 |
| C2orf50/NM182500.2 | KLHL5/NM001007075.2 | SIM2/NM005069.4 |
| C2orf54/NM001085437.2 | KLHL6/NM130446.2 | SIMC1/NM198567.5 |
| C2orf57/NM152614.2 | KLHL7/NM001031710.2 | SIN3A/NM001145357.1 |
| C2orf61/NM001163561.1 | KLHL8/NM001292003.1 | SIN3A/NM001145358.1 |
| C2orf66/NM213608.2 | KLHL8/NM020803.4 | SIN3A/NM015477.2 |
| C2orf68/NM001013649.3 | KLHL9/NM018847.2 | SIN3B/NM015260.3 |
| C2orf69/NM153689.5 | KLK1/NM002257.3 | SIPA1/NM006747.3 |
| C2orf70/NM001105519.2 | KLK10/NM001077500.1 | SIPA1/NM153253.29 |
| C2orf71/NM001029883.2 | KLK10/NM002776.4 | SIPA1L1/NM015556.2 |
| C2orf72/NM001144994.1 | KLK10/NM145888.2 | SIPA1L2/NM020808.4 |
| C2orf73/NM001100396.1 | KLK12/NM145894.1 | SIRPA/NM001040022.1 |
| C2orf74/NM001143959.1 | KLK14/NM001311182.1 | SIRPA/NM001040023.1 |
| C2orf76/NM001017927.3 | KLK15/NM017509.3 | SIRPA/NM080792.2 |
| C2orf76/NM001322331.1 | KLK3/NM001648.2 | SIRPB1/NM006065.3 |
| C2orf78/NM001080474.1 | KLK8/NM007196.3 | SIRPB2/NM001122962.1 |
| C2orf80/NM001099334.2 | KLKB1/NM000892.4 | SIRPD/NM178460.2 |
| C2orf81/NM001316764.1 | KLRB1/NM002258.2 | SIRPG/NM018556.3 |
| C2orf82/NM206895.1 | KLRC1/NM002259.4 | SIRT1/NM012238.4 |
| C2orf83/NM020161.3 | KLRC1/NM213658.2 | SIRT3/NM001017524.2 |
| C2orf88/NM001042519.1 | KLRC2/NM002260.3 | SIRT4/NM012240.2 |
| C2orf88/NM001042520.1 | KLRC3/NM007333.2 | SIRT5/NM012241.4 |
| C2orf88/NM001042521.1 | KLRC4-KLRK1/NM001199805.1 | SIRT6/NM016539.3 |
| C2orf88/NM032321.2 | KLRC4/NM013431.2 | SIRT7/NM016538.2 |
| C2orf91/NM001242815.1 | KLRD1/NM001114396.1 | SIT1/NM014450.2 |
| C3/NM000064.3 | KLRD1/NM002262.3 | SIVA1/NM006427.3 |
| C3AR1/NM004054.2 | KLRF1/NM016523.2 | SIX2/NM016932.4 |
| C3orf14/NM001291942.1 | KLRF2/NM001190765.1 | SIX3/NM005413.3 |
| C3orf14/NM020685.4 | KLRG1/NM005810.3 | SIX5/NM175875.4 |
| C3orf18/NM001171741.2 | KLRG2/NM198508.2 | SIX6/NM007374.2 |
| C3orf18/NM016210.4 | KLRK1/NM007360.3 | SKA1/NM145060.3 |
| C3orf20/NM032137.4 | KMO/NM003679.4 | SKA2/NM182620.3 |
| C3orf22/NM152533.2 | KMT2A/NM005933.3 | SKA3/NM145061.5 |
| C3orf30/NM152539.2 | KMT2B/NM014727.2 | SKAP1/NM003726.3 |
| C3orf33/NM173657.2 | KMT2C/NM170606.2 | SKAP2/NM003930.4 |
| C3orf35/NM178339.2 | KMT2D/NM003482.3 | SKI/NM003036.3 |
| C3orf36/NM025041.2 | KMT2E/NM018682.3 | SKIDA1/NM207371.3 |
| C3orf38/NM173824.3 | KMT5A/NM020382.3 | SKIL/NM001248008.1 |
| C3orf52/NM024616.2 | KMT5B/NM017635.4 | SKIL/NM005414.4 |
| C3orf56/NM001007534.2 | KNCN/NM001322255.1 | SKIV2L/NM006929.4 |
| C3orf58/NM173552.3 | KNDC1/NM152643.6 | SKIV2L2/NM015360.4 |
| C3orf62/NM198562.2 | KNG1/NM000893.3 | SKOR1/NM001258024.1 |
| C3orf70/NM001025266.1 | KNOP1/NM001012991.2 | SKOR2/NM001037802.1 |
| C3orf79/NM001101337.1 | KNSTRN/NM033286.3 | SKP2/NM005983.3 |
| C3orf80/NM001168214.1 | KNTC1/NM014708.4 | SLA/NM001045556.2 |
| C3orf84/NM001080528.2 | KPNA1/NM002264.3 | SLA2/NM032214.3 |
| C4A/NM007293.2 | KPNA2/NM001320611.1 | SLAIN2/NM020846.1 |
| C4B/NM001002029.3 | KPNA2/NM002266.3 | SLAMF1/NM003037.3 |
| C4BPA/NM000715.3 | KPNA3/NM002267.3 | SLAMF6/NM052931.4 |
| C4BPB/NM000716.3 | KPNA4/NM002268.4 | SLAMF7/NM021181.4 |
| C4BPB/NM001017365.2 | KPNA5/NM002269.2 | SLAMF8/NM020125.2 |
| C4BPB/NM001017367.1 | KPNA6/NM012316.4 | SLAMF9/NM033438.3 |
| C4orf19/NM001104629.1 | KPNA7/NM001145715.1 | SLBP/NM006527.3 |
| C4orf19/NM018302.2 | KPNB1/NM002265.5 | SLC10A1/NM003049.3 |
| C4orf22/NM152770.2 | KPRP/NM001025231.1 | SLC10A2/NM000452.2 |
| C4orf26/NM178497.3 | KPTN/NM007059.3 | SLC10A4/NM152679.3 |
| C4orf3/NM001001701.3 | KRAS/NM033360.3 | SLC10A5/NM001010893.2 |
| C4orf32/NM152400.2 | KRBA1/NM001290187.1 | SLC10A6/NM197965.2 |
| C4orf33/NM001099783.1 | KRBA2/NM213597.3 | SLC10A7/NM001029998.5 |
| C4orf33/NM173487.2 | KRBOX1/NM001205272.1 | SLC11A1/NM000578.3 |
| C4orf36/NM144645.3 | KRCC1/NM016618.2 | SLC11A2/NM001174126.1 |
| C4orf45/NM152543.2 | KREMEN1/NM032045.4 | SLC12A2/NM001046.2 |
| C4orf46/NM001008393.3 | KREMEN2/NM172229.2 | SLC12A3/NM000339.2 |
| C4orf47/NM001114357.1 | KRI1/NM023008.3 | SLC12A4/NM005072.4 |
| C4orf48/NM001141936.2 | KRIT1/NM004912.3 | SLC12A6/NM005135.2 |
| C4orf51/NM001080531.1 | KRIT1/NM194454.1 | SLC12A7/NM006598.2 |
| C5/NM001735.2 | KRIT1/NM194455.1 | SLC12A8/NM001195483.1 |
| C5AR1/NM001736.3 | KRR1/NM007043.6 | SLC12A8/NM024628.5 |
| C5AR2/NM001271749.1 | KRT1/NM006121.3 | SLC12A9/NM020246.3 |
| C5AR2/NM001271750.1 | KRT10/NM000421.3 | SLC13A1/NM022444.3 |
| C5AR2/NM018485.2 | KRT12/NM000223.3 | SLC13A2/NM003984.3 |
| C5orf15/NM020199.2 | KRT13/NM153490.2 | SLC13A3/NM022829.5 |
| C5orf22/NM018356.2 | KRT14/NM000526.4 | SLC13A4/NM012450.3 |
| C5orf24/NM001135586.1 | KRT15/NM002275.3 | SLC13A5/NM177550.4 |
| C5orf24/NM152409.3 | KRT18/NM000224.2 | SLC14A1/NM015865.6 |
| C5orf30/NM001316968.1 | KRT18/NM199187.1 | SLC14A2/NM001242692.1 |
| C5orf30/NM001316969.1 | KRT19/NM002276.4 | SLC14A2/NM007163.3 |
| C5orf30/NM033211.3 | KRT2/NM000423.2 | SLC15A1/NM005073.3 |
| C5orf34/NM198566.3 | KRT20/NM019010.2 | SLC15A2/NM021082.3 |
| C5orf38/NM178569.3 | KRT23/NM015515.4 | SLC15A3/NM016582.2 |
| C5orf42/NM023073.3 | KRT24/NM019016.2 | SLC15A4/NM145648.3 |
| C5orf45/NM016175.3 | KRT25/NM181534.3 | SLC15A5/NM001170798.1 |
| C5orf46/NM206966.2 | KRT26/NM181539.4 | SLC16A1/NM001166496.1 |
| C5orf47/NM001144954.1 | KRT27/NM181537.3 | SLC16A1/NM003051.3 |
| C5orf49/NM001089584.2 | KRT28/NM181535.3 | SLC16A10/NM018593.4 |
| C5orf51/NM175921.5 | KRT3/NM057088.2 | SLC16A11/NM153357.1 |
| C5orf52/NM001145132.1 | KRT31/NM002277.2 | SLC16A12/NM213606.3 |
| C5orf58/NM001102609.2 | KRT32/NM002278.3 | SLC16A13/NM201566.2 |
| C5orf58/NM001305394.1 | KRT33A/NM004138.3 | SLC16A14/NM152527.4 |
| C5orf63/NM001164478.1 | KRT33B/NM002279.4 | SLC16A3/NM001042422.2 |
| C5orf64/NM173667.3 | KRT34/NM021013.3 | SLC16A3/NM001042423.2 |
| C5orf66/NM001277348.1 | KRT35/NM002280.4 | SLC16A3/NM001206951.1 |
| C5orf67/NM001287053.1 | KRT36/NM003771.4 | SLC16A3/NM001206952.1 |
| C6/NM001115131.2 | KRT37/NM003770.4 | SLC16A3/NM004207.3 |
| C6orf1/NM178508.4 | KRT38/NM006771.3 | SLC16A4/NM004696.2 |
| C6orf106/NM024294.3 | KRT39/NM213656.3 | SLC16A5/NM001271765.1 |
| C6orf118/NM144980.3 | KRT40/NM182497.3 | SLC16A5/NM004695.3 |
| C6orf120/NM001029863.2 | KRT5/NM000424.3 | SLC16A6/NM001174166.1 |
| C6orf132/NM001164446.2 | KRT6A/NM005554.3 | SLC16A7/NM001270622.1 |
| C6orf136/NM001109938.2 | KRT6B/NM005555.3 | SLC16A7/NM004731.4 |
| C6orf141/NM001145652.1 | KRT6C/NM173086.4 | SLC16A8/NM013356.2 |
| C6orf15/NM014070.2 | KRT7/NM005556.3 | SLC16A9/NM001323981.1 |
| C6orf163/NM001010868.2 | KRT71/NM033448.2 | SLC17A2/NM001286123.1 |
| C6orf201/NM001085401.2 | KRT72/NM080747.2 | SLC17A3/NM001098486.1 |
| C6orf203/NM016487.4 | KRT73/NM175068.2 | SLC17A4/NM005495.2 |
| C6orf222/NM001010903.4 | KRT74/NM175053.3 | SLC17A5/NM012434.4 |
| C6orf223/NM153246.5 | KRT75/NM004693.2 | SLC17A6/NM020346.2 |
| C6orf226/NM001008739.1 | KRT76/NM015848.4 | SLC17A7/NM020309.3 |
| C6orf229/NM001282492.1 | KRT77/NM175078.2 | SLC17A9/NM022082.3 |
| C6orf25/NM138272.2 | KRT78/NM173352.3 | SLC18A2/NM003054.4 |
| C6orf47/NM021184.3 | KRT79/NM175834.2 | SLC18A3/NM003055.2 |
| C6orf48/NM001287488.1 | KRT8/NM001256293.1 | SLC18B1/NM052831.2 |
| C6orf52/NM001145020.1 | KRT8/NM002273.3 | SLC19A1/NM194255.2 |
| C6orf58/NM001010905.2 | KRT80/NM182507.2 | SLC19A2/NM006996.2 |
| C6orf62/NM030939.4 | KRT81/NM002281.3 | SLC19A3/NM025243.3 |
| C6orf89/NM152734.3 | KRT82/NM033033.3 | SLC1A1/NM004170.5 |
| C6orf99/NM001195032.1 | KRT83/NM002282.3 | SLC1A2/NM004171.3 |
| C6orf99/NM001302841.1 | KRT84/NM033045.3 | SLC1A3/NM004172.4 |
| C7/NM000587.2 | KRT85/NM002283.3 | SLC1A4/NM003038.4 |
| C7orf25/NM024054.2 | KRT86/NM001320198.1 | SLC1A5/NM005628.2 |
| C7orf26/NM024067.3 | KRT9/NM000226.3 | SLC1A6/NM005071.2 |
| C7orf31/NM138811.3 | KRTAP1-1/NM030967.2 | SLC1A7/NM006671.5 |
| C7orf33/NM145304.3 | KRTAP1-3/NM030966.1 | SLC20A1/NM005415.4 |
| C7orf34/NM178829.4 | KRTAP1-4/NM001257305.1 | SLC20A2/NM001257180.1 |
| C7orf43/NM018275.4 | KRTAP1-5/NM031957.1 | SLC20A2/NM001257181.1 |
| C7orf49/NM024033.3 | KRTAP10-1/NM198691.2 | SLC20A2/NM006749.4 |
| C7orf50/NM032350.5 | KRTAP10-10/NM181688.2 | SLC22A1/NM003057.2 |
| C7orf55-LUC7L2/NM001244584.2 | KRTAP10-11/NM198692.2 | SLC22A10/NM001039752.3 |
| C7orf57/NM001100159.2 | KRTAP10-12/NM198699.1 | SLC22A11/NM018484.3 |
| C7orf61/NM001004323.1 | KRTAP10-2/NM198693.3 | SLC22A12/NM144585.3 |
| C7orf62/NM152706.3 | KRTAP10-3/NM198696.2 | SLC22A13/NM004256.3 |
| C7orf65/NM001123065.1 | KRTAP10-4/NM198687.2 | SLC22A14/NM004803.4 |
| C7orf66/NM001024607.2 | KRTAP10-5/NM198694.3 | SLC22A15/NM018420.2 |
| C7orf69/NM025031.2 | KRTAP10-6/NM198688.3 | SLC22A16/NM033125.3 |
| C7orf71/NM001145531.2 | KRTAP10-7/NM198689.2 | SLC22A17/NM020372.3 |
| C7orf72/NM001161834.2 | KRTAP10-8/NM198695.2 | SLC22A18/NM002555.5 |
| C7orf73/NM001130929.1 | KRTAP10-9/NM198690.2 | SLC22A18/NM183233.2 |
| C7orf76/NM001201451.1 | KRTAP11-1/NM175858.2 | SLC22A18AS/NM007105.3 |
| C7orf77/NM001024603.1 | KRTAP12-1/NM181686.1 | SLC22A2/NM003058.3 |
| C8A/NM000562.2 | KRTAP12-2/NM181684.2 | SLC22A23/NM015482.1 |
| C8G/NM000606.2 | KRTAP12-3/NM198697.2 | SLC22A24/NM001136506.2 |
| C8orf31/NM173687.3 | KRTAP12-4/NM198698.1 | SLC22A25/NM199352.3 |
| C8orf33/NM023080.2 | KRTAP13-1/NM181599.2 | SLC22A3/NM021977.3 |
| C8orf34/NM052958.2 | KRTAP13-2/NM181621.3 | SLC22A31/NM001242757.1 |
| C8orf37/NM177965.3 | KRTAP13-3/NM181622.1 | SLC22A4/NM003059.2 |
| C8orf4/NM020130.4 | KRTAP13-4/NM181600.1 | SLC22A5/NM003060.3 |
| C8orf44-SGK3/NM001204173.1 | KRTAP15-1/NM181623.1 | SLC22A7/NM153320.2 |
| C8orf46/NM152765.3 | KRTAP16-1/NM001146182.1 | SLC22A8/NM004254.3 |
| C8orf48/NM001007090.2 | KRTAP17-1/NM031964.1 | SLC22A9/NM080866.2 |
| C8orf59/NM001099670.2 | KRTAP19-1/NM181607.1 | SLC23A1/NM152685.3 |
| C8orf74/NM001040032.1 | KRTAP19-2/NM181608.1 | SLC23A2/NM203327.1 |
| C8orf76/NM032847.2 | KRTAP19-3/NM181609.3 | SLC23A3/NM001144890.1 |
| C8orf82/NM001001795.1 | KRTAP19-4/NM181610.1 | SLC24A1/NM004727.2 |
| C8orf86/NM207412.2 | KRTAP19-5/NM181611.1 | SLC24A2/NM020344.3 |
| C8orf87/NM001242668.1 | KRTAP19-6/NM181612.3 | SLC24A3/NM020689.3 |
| C8orf88/NM001190972.1 | KRTAP19-7/NM181614.1 | SLC24A4/NM153647.3 |
| C8orf89/NM001243237.1 | KRTAP19-8/NM001099219.1 | SLC24A5/NM205850.2 |
| C9/NM001737.3 | KRTAP2-1/NM001123387.1 | SLC25A1/NM005984.4 |
| C9orf106/NM001012715.3 | KRTAP2-2/NM033032.2 | SLC25A10/NM012140.4 |
| C9orf114/NM016390.3 | KRTAP2-3/NM001165252.1 | SLC25A11/NM003562.4 |
| C9orf116/NM001048265.1 | KRTAP2-4/NM033184.3 | SLC25A12/NM003705.4 |
| C9orf129/NM001098808.1 | KRTAP20-1/NM181615.1 | SLC25A13/NM014251.2 |
| C9orf131/NM203299.3 | KRTAP20-2/NM181616.1 | SLC25A15/NM014252.3 |
| C9orf135/NM001010940.2 | KRTAP20-3/NM001128077.1 | SLC25A16/NM152707.3 |
| C9orf139/NM207511.2 | KRTAP21-1/NM181619.1 | SLC25A17/NM006358.3 |
| C9orf142/NM183241.1 | KRTAP21-2/NM181617.1 | SLC25A18/NM001303484.1 |
| C9orf152/NM001012993.2 | KRTAP21-3/NM001164435.1 | SLC25A19/NM001126121.1 |
| C9orf153/NM001276367.1 | KRTAP22-1/NM181620.1 | SLC25A2/NM031947.3 |
| C9orf153/NM001276368.1 | KRTAP22-2/NM001164434.1 | SLC25A20/NM000387.5 |
| C9orf16/NM024112.3 | KRTAP23-1/NM181624.1 | SLC25A21/NM030631.3 |
| C9orf163/NM152571.2 | KRTAP24-1/NM001085455.2 | SLC25A22/NM001191060.1 |
| C9orf170/NM001001709.2 | KRTAP25-1/NM001128598.1 | SLC25A22/NM001191061.1 |
| C9orf172/NM001080482.2 | KRTAP26-1/NM203405.1 | SLC25A22/NM024698.5 |
| C9orf24/NM032596.3 | KRTAP27-1/NM001077711.1 | SLC25A23/NM024103.2 |
| C9orf3/NM001193329.1 | KRTAP29-1/NM001257309.1 | SLC25A24/NM013386.4 |
| C9orf40/NM017998.2 | KRTAP3-1/NM031958.1 | SLC25A25/NM052901.4 |
| C9orf43/NM001278629.1 | KRTAP3-2/NM031959.2 | SLC25A26/NM173471.3 |
| C9orf43/NM152786.2 | KRTAP3-3/NM033185.2 | SLC25A27/NM004277.4 |
| C9orf47/NM001001938.3 | KRTAP4-1/NM033060.2 | SLC25A28/NM031212.3 |
| C9orf50/NM199350.3 | KRTAP4-11/NM033059.3 | SLC25A29/NM001039355.2 |
| C9orf57/NM001128618.1 | KRTAP4-12/NM031854.2 | SLC25A3/NM005888.3 |
| C9orf62/NM173520.2 | KRTAP4-2/NM033062.3 | SLC25A30/NM001010875.3 |
| C9orf64/NM032307.4 | KRTAP4-3/NM033187.1 | SLC25A31/NM031291.3 |
| C9orf66/NM152569.2 | KRTAP4-4/NM032524.1 | SLC25A32/NM030780.4 |
| C9orf69/NM001256526.1 | KRTAP4-5/NM033188.3 | SLC25A33/NM032315.2 |
| C9orf69/NM152833.2 | KRTAP4-6/NM030976.1 | SLC25A34/NM207348.2 |
| C9orf72/NM001256054.2 | KRTAP5-1/NM001005922.1 | SLC25A35/NM001320870.1 |
| C9orf72/NM018325.4 | KRTAP5-10/NM001012710.1 | SLC25A38/NM017875.2 |
| C9orf78/NM016520.2 | KRTAP5-11/NM001005405.2 | SLC25A39/NM016016.3 |
| C9orf84/NM173521.4 | KRTAP5-2/NM001004325.1 | SLC25A4/NM001151.3 |
| C9orf85/NM182505.3 | KRTAP5-3/NM001012708.2 | SLC25A40/NM018843.3 |
| C9orf91/NM153045.3 | KRTAP5-5/NM001001480.2 | SLC25A41/NM173637.3 |
| C9orf92/NM001271829.1 | KRTAP5-6/NM001012416.1 | SLC25A42/NM001321544.1 |
| CA1/NM001164830.1 | KRTAP5-7/NM001012503.1 | SLC25A42/NM178526.4 |
| CA1/NM001738.4 | KRTAP5-8/NM021046.2 | SLC25A44/NM014655.3 |
| CA10/NM001082533.1 | KRTAP5-9/NM005553.3 | SLC25A45/NM182556.3 |
| CA10/NM001082534.1 | KRTAP6-1/NM181602.1 | SLC25A46/NM138773.2 |
| CA10/NM020178.4 | KRTAP6-2/NM181604.1 | SLC25A47/NM207117.2 |
| CA11/NM001217.4 | KRTAP6-3/NM181605.3 | SLC25A51/NM033412.3 |
| CA12/NM001218.4 | KRTAP7-1/NM181606.2 | SLC25A52/NM001034172.2 |
| CA13/NM198584.2 | KRTAP8-1/NM175857.3 | SLC26A1/NM213613.3 |
| CA14/NM012113.2 | KRTAP9-1/NM001190460.1 | SLC26A2/NM000112.3 |
| CA2/NM000067.2 | KRTAP9-2/NM031961.2 | SLC26A3/NM000111.2 |
| CA3/NM005181.3 | KRTAP9-4/NM033191.2 | SLC26A4/NM000441.1 |
| CA4/NM000717.3 | KRTAP9-6/NM001277331.1 | SLC26A5/NM198999.2 |
| CA6/NM001215.3 | KRTAP9-7/NM001277332.1 | SLC26A6/NM022911.2 |
| CA7/NM005182.2 | KRTAP9-8/NM031963.2 | SLC26A7/NM001282356.1 |
| CA8/NM004056.5 | KRTAP9-9/NM030975.2 | SLC26A7/NM052832.3 |
| CA9/NM001216.2 | KRTCAP2/NM173852.3 | SLC26A8/NM001193476.1 |
| CAAP1/NM024828.3 | KRTCAP3/NM001321325.1 | SLC26A8/NM052961.3 |
| CAB39/NM001130850.1 | KRTDAP/NM207392.2 | SLC26A9/NM134325.2 |
| CAB39/NM016289.3 | KTI12/NM138417.2 | SLC27A1/NM198580.2 |
| CAB39L/NM001287337.1 | KTN1/NM001079521.1 | SLC27A2/NM003645.3 |
| CAB39L/NM001287338.1 | KXD1/NM024069.3 | SLC27A3/NM024330.1 |
| CAB39L/NM030925.3 | KY/NM178554.4 | SLC27A4/NM005094.3 |
| CABIN1/NM001199281.1 | KYAT1/NM004059.4 | SLC27A5/NM012254.2 |
| CABIN1/NM012295.3 | KYAT3/NM001008661.2 | SLC27A6/NM001317984.1 |
| CABLES1/NM001100619.2 | KYNU/NM003937.2 | SLC28A3/NM001199633.1 |
| CABLES2/NM031215.2 | L1TD1/NM019079.4 | SLC28A3/NM022127.2 |
| CABP2/NM016366.2 | L2HGDH/NM024884.2 | SLC29A1/NM001078175.2 |
| CABP4/NM145200.3 | L3MBTL1/NM015478.6 | SLC29A1/NM001078177.1 |
| CABP5/NM019855.4 | L3MBTL2/NM031488.4 | SLC29A2/NM001300868.1 |
| CABP7/NM182527.2 | LACC1/NM001128303.1 | SLC29A2/NM001532.2 |
| CABS1/NM033122.3 | LACC1/NM153218.2 | SLC29A3/NM018344.5 |
| CABYR/NM012189.3 | LACE1/NM145315.4 | SLC29A4/NM153247.2 |
| CACFD1/NM017586.3 | LACTB/NM032857.4 | SLC2A1/NM006516.2 |
| CACHD1/NM020925.2 | LACTB2/NM016027.2 | SLC2A10/NM030777.3 |
| CACNA1A/NM001127222.1 | LACTBL1/NM001289974.1 | SLC2A11/NM001024938.3 |
| CACNA1B/NM000718.3 | LAD1/NM005558.3 | SLC2A12/NM145176.2 |
| CACNA1C/NM001129827.1 | LAG3/NM002286.5 | SLC2A13/NM052885.3 |
| CACNA1D/NM000720.3 | LAIR1/NM002287.5 | SLC2A14/NM001286233.1 |
| CACNA1E/NM000721.3 | LAIR2/NM002288.5 | SLC2A2/NM000340.1 |
| CACNA1H/NM021098.2 | LALBA/NM002289.2 | SLC2A3/NM006931.2 |
| CACNA1I/NM021096.3 | LAMA2/NM000426.3 | SLC2A4/NM001042.2 |
| CACNA1S/NM000069.2 | LAMA3/NM198129.2 | SLC2A4RG/NM020062.3 |
| CACNA2D1/NM000722.3 | LAMA4/NM001105207.2 | SLC2A5/NM003039.2 |
| CACNA2D2/NM006030.3 | LAMA5/NM005560.4 | SLC2A6/NM017585.3 |
| CACNA2D3/NM018398.2 | LAMB1/NM002291.2 | SLC2A7/NM207420.2 |
| CACNA2D4/NM172364.4 | LAMB2/NM002292.3 | SLC2A8/NM014580.4 |
| CACNB1/NM000723.4 | LAMB3/NM000228.2 | SLC2A9/NM020041.2 |
| CACNB2/NM201571.3 | LAMB3/NM001017402.1 | SLC30A1/NM021194.2 |
| CACNB3/NM000725.3 | LAMB3/NM001127641.1 | SLC30A10/NM018713.2 |
| CACNB4/NM001005746.2 | LAMB4/NM007356.2 | SLC30A2/NM001004434.2 |
| CACNG1/NM000727.3 | LAMC1/NM002293.3 | SLC30A3/NM003459.4 |
| CACNG2/NM006078.3 | LAMC2/NM005562.2 | SLC30A4/NM013309.5 |
| CACNG3/NM006539.3 | LAMC3/NM006059.3 | SLC30A5/NM022902.4 |
| CACNG4/NM014405.3 | LAMP1/NM005561.3 | SLC30A6/NM017964.3 |
| CACNG5/NM145811.2 | LAMP3/NM014398.3 | SLC30A7/NM001144884.1 |
| CACNG6/NM145814.1 | LAMP5/NM012261.3 | SLC30A7/NM133496.4 |
| CACNG7/NM031896.4 | LAMTOR2/NM014017.3 | SLC30A8/NM173851.2 |
| CACNG8/NM031895.5 | LAMTOR3/NM021970.3 | SLC30A9/NM006345.3 |
| CACTIN/NM001080543.1 | LAMTOR4/NM001008395.3 | SLC31A1/NM001859.3 |
| CACUL1/NM153810.4 | LAMTOR5/NM006402.2 | SLC31A2/NM001860.2 |
| CACYBP/NM014412.2 | LANCL1/NM001136574.1 | SLC32A1/NM080552.2 |
| CAD/NM004341.4 | LANCL1/NM001136575.1 | SLC33A1/NM004733.3 |
| CADM1/NM001098517.1 | LANCL1/NM006055.2 | SLC34A1/NM003052.4 |
| CADM3/NM021189.3 | LANCL2/NM018697.3 | SLC34A3/NM001177316.1 |
| CADM4/NM145296.1 | LAP3/NM015907.2 | SLC34A3/NM080877.2 |
| CADPS/NM183394.2 | LAPTM4A/NM014713.4 | SLC35A1/NM006416.4 |
| CADPS2/NM001009571.3 | LAPTM4B/NM018407.4 | SLC35A3/NM012243.2 |
| CAGE1/NM205864.2 | LAPTM5/NM006762.2 | SLC35A4/NM080670.3 |
| CALB1/NM004929.3 | LARGE/NM004737.4 | SLC35A5/NM017945.3 |
| CALB2/NM001740.4 | LARP1/NM015315.4 | SLC35B1/NM005827.2 |
| CALCB/NM000728.3 | LARP1B/NM018078.3 | SLC35B2/NM178148.3 |
| CALCOCO1/NM020898.2 | LARP4/NM052879.4 | SLC35B3/NM015948.4 |
| CALCOCO2/NM005831.4 | LARP4B/NM015155.2 | SLC35B4/NM032826.4 |
| CALCR/NM001164738.1 | LARP6/NM018357.3 | SLC35C1/NM018389.4 |
| CALCRL/NM005795.5 | LARP7/NM015454.2 | SLC35C2/NM001281460.1 |
| CALD1/NM004342.6 | LARP7/NM016648.3 | SLC35D1/NM015139.2 |
| CALHM1/NM001001412.3 | LARS/NM020117.10 | SLC35D2/NM007001.2 |
| CALHM2/NM015916.4 | LARS2/NM015340.3 | SLC35D3/NM001008783.2 |
| CALHM3/NM001129742.1 | LASP1/NM006148.3 | SLC35E1/NM024881.4 |
| CALM1/NM006888.4 | LAT/NM014387.3 | SLC35E2/NM182838.2 |
| CALM2/NM001743.5 | LAT2/NM014146.3 | SLC35E2B/NM001290264.1 |
| CALM3/NM005184.2 | LATS1/NM004690.3 | SLC35E3/NM018656.2 |
| CALML3/NM005185.3 | LATS2/NM014572.2 | SLC35E4/NM001001479.3 |
| CALML5/NM017422.4 | LAX1/NM017773.3 | SLC35F1/NM001029858.3 |
| CALML6/NM138705.2 | LAYN/NM178834.4 | SLC35F3/NM173508.3 |
| CALN1/NM001017440.2 | LBH/NM030915.3 | SLC35F4/NM001306087.1 |
| CALR3/NM145046.4 | LBHD1/NM024099.3 | SLC35F5/NM025181.3 |
| CALU/NM001130674.2 | LBP/NM004139.4 | SLC35F6/NM017877.3 |
| CALY/NM015722.3 | LBR/NM002296.3 | SLC35G1/NM153226.2 |
| CAMK1/NM003656.4 | LBR/NM194442.2 | SLC35G2/NM001097600.1 |
| CAMK1D/NM153498.3 | LBX1/NM006562.4 | SLC35G3/NM152462.2 |
| CAMK1G/NM020439.2 | LBX2/NM001282430.1 | SLC35G4/NM001282300.1 |
| CAMK2A/NM015981.3 | LCA5/NM181714.3 | SLC35G5/NM054028.1 |
| CAMK2B/NM172078.2 | LCA5L/NM152505.3 | SLC36A1/NM078483.3 |
| CAMK2D/NM001221.3 | LCAT/NM000229.1 | SLC36A3/NM181774.3 |
| CAMK2G/NM172171.2 | LCE1A/NM178348.2 | SLC37A1/NM018964.3 |
| CAMK2N1/NM018584.5 | LCE1B/NM178349.1 | SLC37A2/NM198277.2 |
| CAMK2N2/NM033259.2 | LCE1D/NM178352.2 | SLC37A3/NM207113.2 |
| CAMK4/NM001323374.1 | LCE1E/NM178353.1 | SLC37A4/NM001164278.1 |
| CAMK4/NM001323375.1 | LCE1F/NM178354.2 | SLC38A1/NM001278387.1 |
| CAMK4/NM001744.5 | LCE2A/NM178428.3 | SLC38A1/NM001278389.1 |
| CAMKK1/NM032294.2 | LCE2B/NM014357.4 | SLC38A10/NM138570.3 |
| CAMKK1/NM172206.1 | LCE2C/NM178429.3 | SLC38A2/NM018976.4 |
| CAMKK2/NM001270485.1 | LCE2D/NM178430.3 | SLC38A3/NM006841.5 |
| CAMKK2/NM006549.3 | LCE3A/NM178431.1 | SLC38A4/NM018018.4 |
| CAMKMT/NM024766.4 | LCE3B/NM178433.1 | SLC38A6/NM153811.2 |
| CAMKV/NM024046.4 | LCE3C/NM178434.2 | SLC38A7/NM018231.2 |
| CAMLG/NM001745.3 | LCE3D/NM032563.1 | SLC38A8/NM001080442.2 |
| CAMP/NM004345.4 | LCE3E/NM178435.3 | SLC38A9/NM173514.3 |
| CAMSAP1/NM015447.3 | LCE4A/NM178356.3 | SLC39A1/NM001271957.1 |
| CAMSAP2/NM203459.2 | LCE5A/NM178438.4 | SLC39A1/NM001271958.1 |
| CAMSAP3/NM020902.1 | LCE6A/NM001128600.1 | SLC39A1/NM001271959.1 |
| CAMTA1/NM015215.3 | LCK/NM001042771.2 | SLC39A1/NM001271960.1 |
| CAMTA2/NM015099.3 | LCK/NM005356.4 | SLC39A1/NM014437.4 |
| CAND1/NM018448.4 | LCLAT1/NM001002257.2 | SLC39A10/NM001127257.1 |
| CAND2/NM001162499.1 | LCMT1/NM016309.2 | SLC39A10/NM020342.2 |
| CANT1/NM001159772.1 | LCMT2/NM014793.4 | SLC39A11/NM139177.3 |
| CANX/NM001024649.1 | LCN10/NM001001712.2 | SLC39A12/NM152725.3 |
| CAP1/NM001105530.1 | LCN12/NM178536.3 | SLC39A13/NM152264.4 |
| CAP2/NM006366.2 | LCN15/NM203347.1 | SLC39A14/NM015359.4 |
| CAPG/NM001256139.1 | LCN2/NM005564.4 | SLC39A2/NM014579.3 |
| CAPG/NM001320732.1 | LCN6/NM198946.2 | SLC39A3/NM144564.4 |
| CAPG/NM001320733.1 | LCN8/NM178469.3 | SLC39A4/NM130849.3 |
| CAPG/NM001747.3 | LCNL1/NM207510.3 | SLC39A5/NM001135195.1 |
| CAPN10/NM023083.3 | LCOR/NM001170765.1 | SLC39A5/NM173596.2 |
| CAPN11/NM007058.3 | LCOR/NM032440.3 | SLC39A6/NM012319.3 |
| CAPN12/NM144691.4 | LCP1/NM002298.4 | SLC39A7/NM001077516.1 |
| CAPN13/NM144575.2 | LCP2/NM005565.3 | SLC39A8/NM001135146.1 |
| CAPN14/NM001145122.1 | LCT/NM002299.3 | SLC39A8/NM022154.5 |
| CAPN15/NM005632.2 | LCTL/NM207338.3 | SLC39A9/NM018375.4 |
| CAPN2/NM001748.4 | LDAH/NM021925.3 | SLC3A1/NM000341.3 |
| CAPN3/NM000070.2 | LDB1/NM003893.4 | SLC3A2/NM002394.5 |
| CAPN5/NM004055.4 | LDB2/NM001290.4 | SLC41A2/NM032148.3 |
| CAPN7/NM014296.2 | LDB3/NM001080114.1 | SLC41A3/NM001008485.1 |
| CAPN8/NM001143962.1 | LDHAL6A/NM001144071.1 | SLC43A1/NM001198810.1 |
| CAPN9/NM006615.2 | LDHAL6B/NM033195.2 | SLC43A1/NM003627.5 |
| CAPNS1/NM001003962.2 | LDHB/NM001174097.2 | SLC43A2/NM152346.2 |
| CAPNS1/NM001302632.1 | LDHB/NM002300.7 | SLC43A3/NM014096.3 |
| CAPNS2/NM032330.1 | LDHC/NM017448.3 | SLC43A3/NM017611.2 |
| CAPRIN1/NM005898.4 | LDHD/NM194436.2 | SLC43A3/NM199329.2 |
| CAPRIN2/NM023925.4 | LDLR/NM000527.4 | SLC44A1/NM080546.4 |
| CAPS/NM004058.4 | LDLRAD2/NM001013693.2 | SLC44A2/NM020428.3 |
| CAPSL/NM001042625.1 | LDLRAD3/NM174902.3 | SLC44A3/NM152369.4 |
| CAPSL/NM144647.3 | LDLRAD4/NM181481.4 | SLC44A4/NM025257.2 |
| CAPZA2/NM006136.2 | LDLRAP1/NM015627.2 | SLC44A5/NM152697.4 |
| CAPZA3/NM033328.2 | LDOC1L/NM032287.2 | SLC45A1/NM001080397.2 |
| CAPZB/NM004930.4 | LEAP2/NM052971.2 | SLC45A3/NM033102.2 |
| CARD10/NM014550.3 | LECT1/NM007015.2 | SLC45A4/NM001080431.2 |
| CARD11/NM032415.5 | LEF1/NM016269.4 | SLC46A1/NM080669.5 |
| CARD14/NM024110.4 | LEFTY1/NM020997.3 | SLC46A2/NM033051.3 |
| CARD16/NM001017534.1 | LEFTY2/NM003240.3 | SLC46A3/NM181785.3 |
| CARD17/NM001007232.1 | LEKR1/NM001004316.2 | SLC47A1/NM018242.2 |
| CARD18/NM021571.3 | LELP1/NM001010857.2 | SLC47A2/NM152908.3 |
| CARD19/NM032310.4 | LEMD2/NM181336.3 | SLC48A1/NM017842.2 |
| CARD6/NM032587.3 | LEMD3/NM014319.4 | SLC4A1/NM000342.3 |
| CARD8/NM014959.3 | LENEP/NM018655.2 | SLC4A11/NM032034.3 |
| CARD9/NM052813.4 | LENG1/NM024316.2 | SLC4A1AP/NM018158.2 |
| CARF/NM001104586.2 | LENG8/NM052925.3 | SLC4A2/NM001199692.1 |
| CARHSP1/NM001042476.2 | LENG9/NM001301782.1 | SLC4A2/NM003040.3 |
| CARHSP1/NM001278260.1 | LEO1/NM138792.3 | SLC4A3/NM201574.2 |
| CARHSP1/NM001278261.1 | LEP/NM000230.2 | SLC4A4/NM001098484.2 |
| CARHSP1/NM001278263.1 | LEPROTL1/NM015344.2 | SLC4A5/NM021196.3 |
| CARHSP1/NM001278264.1 | LETM1/NM012318.2 | SLC4A7/NM003615.4 |
| CARHSP1/NM001278265.1 | LETM2/NM001199659.2 | SLC4A8/NM001039960.2 |
| CARM1/NM199141.1 | LETMD1/NM015416.4 | SLC4A9/NM031467.2 |
| CARMIL2/NM001317026.1 | LEUTX/NM001143832.1 | SLC51A/NM152672.5 |
| CARMIL3/NM138360.3 | LEXM/NM152607.2 | SLC51B/NM178859.3 |
| CARNMT1/NM001320497.1 | LFNG/NM001040167.1 | SLC52A1/NM001104577.1 |
| CARNS1/NM020811.1 | LGALS12/NM033101.3 | SLC52A2/NM001253815.1 |
| CARS/NM001751.5 | LGALS13/NM013268.2 | SLC52A2/NM001253816.1 |
| CARS2/NM024537.3 | LGALS14/NM203471.1 | SLC52A2/NM024531.4 |
| CARTPT/NM004291.3 | LGALS16/NM001190441.2 | SLC52A3/NM033409.3 |
| CASC1/NM018272.4 | LGALS2/NM006498.2 | SLC5A1/NM000343.3 |
| CASC10/NM001010911.2 | LGALS3/NM002306.3 | SLC5A10/NM152351.4 |
| CASC3/NM007359.4 | LGALS3BP/NM005567.3 | SLC5A11/NM052944.4 |
| CASC5/NM170589.4 | LGALS4/NM006149.3 | SLC5A2/NM003041.3 |
| CASD1/NM022900.4 | LGALS7/NM002307.3 | SLC5A3/NM006933.6 |
| CASKIN1/NM020764.3 | LGALS7B/NM001042507.3 | SLC5A4/NM014227.2 |
| CASKIN2/NM020753.4 | LGALS8/NM006499.4 | SLC5A5/NM000453.2 |
| CASP1/NM001257118.2 | LGALS8/NM201545.2 | SLC5A6/NM021095.2 |
| CASP10/NM032977.3 | LGALS9/NM009587.2 | SLC5A7/NM001305005.2 |
| CASP14/NM012114.2 | LGALS9B/NM001042685.1 | SLC5A8/NM145913.3 |
| CASP2/NM032982.3 | LGALS9C/NM001040078.2 | SLC5A9/NM001011547.2 |
| CASP3/NM004346.3 | LGALSL/NM014181.2 | SLC6A1/NM003042.3 |
| CASP4/NM001225.3 | LGI1/NM005097.3 | SLC6A11/NM014229.2 |
| CASP5/NM004347.3 | LGI2/NM018176.3 | SLC6A12/NM001122847.2 |
| CASP6/NM001226.3 | LGI4/NM139284.2 | SLC6A12/NM001122848.2 |
| CASP7/NM001227.4 | LGMN/NM001008530.2 | SLC6A12/NM003044.4 |
| CASP7/NM001267056.1 | LGR4/NM018490.2 | SLC6A13/NM016615.4 |
| CASP8/NM033355.3 | LGR5/NM003667.3 | SLC6A15/NM182767.5 |
| CASP8AP2/NM001137668.1 | LGR6/NM021636.2 | SLC6A16/NM014037.2 |
| CASP9/NM001229.4 | LHB/NM000894.2 | SLC6A17/NM001010898.2 |
| CASQ1/NM001231.4 | LHCGR/NM000233.3 | SLC6A18/NM182632.2 |
| CASQ2/NM001232.3 | LHFP/NM005780.2 | SLC6A19/NM001003841.2 |
| CASR/NM000388.3 | LHFPL2/NM005779.2 | SLC6A2/NM001043.3 |
| CASS4/NM001164116.1 | LHFPL4/NM198560.2 | SLC6A2/NM001172501.1 |
| CASS4/NM020356.3 | LHFPL5/NM182548.3 | SLC6A20/NM020208.3 |
| CAST/NM173060.3 | LHPP/NM022126.3 | SLC6A3/NM001044.4 |
| CASZ1/NM001079843.2 | LHX1/NM005568.4 | SLC6A4/NM001045.5 |
| CAT/NM001752.3 | LHX3/NM014564.4 | SLC6A6/NM003043.5 |
| CATIP/NM198559.1 | LHX4/NM033343.3 | SLC6A7/NM014228.4 |
| CATSPER1/NM053054.3 | LHX5/NM022363.2 | SLC6A9/NM001024845.2 |
| CATSPER2/NM172095.2 | LHX6/NM014368.4 | SLC7A1/NM003045.4 |
| CATSPER3/NM178019.2 | LHX8/NM001256114.1 | SLC7A10/NM019849.2 |
| CATSPER4/NM198137.1 | LIAS/NM006859.3 | SLC7A11/NM014331.3 |
| CATSPERB/NM024764.3 | LIF/NM002309.4 | SLC7A13/NM138817.2 |
| CATSPERD/NM152784.3 | LIFR/NM001127671.1 | SLC7A4/NM004173.2 |
| CATSPERG/NM021185.4 | LIFR/NM002310.5 | SLC7A5/NM003486.5 |
| CAV1/NM001753.4 | LIG1/NM000234.2 | SLC7A6/NM003983.5 |
| CAV2/NM001233.4 | LIG3/NM013975.3 | SLC7A6OS/NM032178.2 |
| CBARP/NM152769.2 | LIG4/NM001098268.1 | SLC7A7/NM001126105.2 |
| CBFA2T2/NM001032999.2 | LIG4/NM002312.3 | SLC7A7/NM001126106.2 |
| CBFA2T3/NM005187.5 | LIG4/NM206937.1 | SLC7A8/NM012244.3 |
| CBFB/NM001755.2 | LILRA1/NM006863.3 | SLC7A9/NM001243036.1 |
| CBL/NM005188.3 | LILRA2/NM006866.3 | SLC8A1/NM021097.2 |
| CBLB/NM170662.4 | LILRA4/NM012276.4 | SLC8A2/NM015063.2 |
| CBLC/NM012116.3 | LILRA5/NM021250.3 | SLC8A3/NM033262.4 |
| CBLN1/NM004352.3 | LILRA6/NM024318.3 | SLC8B1/NM024959.2 |
| CBLN2/NM182511.3 | LILRB1/NM001081637.2 | SLC9A1/NM003047.4 |
| CBLN3/NM001039771.2 | LILRB2/NM005874.4 | SLC9A2/NM003048.4 |
| CBLN4/NM080617.5 | LILRB3/NM006864.3 | SLC9A3/NM004174.3 |
| CBR1/NM001757.3 | LILRB4/NM001278426.3 | SLC9A3R1/NM004252.4 |
| CBR3/NM001236.3 | LILRB5/NM006840.4 | SLC9A3R2/NM001130012.2 |
| CBR4/NM032783.4 | LIM2/NM001161748.1 | SLC9A5/NM004594.2 |
| CBS/NM000071.2 | LIMA1/NM016357.4 | SLC9A8/NM015266.2 |
| CBS/NM001178008.2 | LIMCH1/NM014988.3 | SLC9A9/NM173653.3 |
| CBSL/NM001321073.1 | LIMD1/NM014240.2 | SLC9B1/NM139173.3 |
| CBWD1/NM018491.3 | LIMD2/NM030576.3 | SLC9B2/NM178833.5 |
| CBWD3/NM201453.3 | LIME1/NM017806.3 | SLC9C1/NM183061.2 |
| CBWD6/NM001085457.1 | LIMK1/NM002314.3 | SLCO1A2/NM021094.3 |
| CBX1/NM001127228.1 | LIMK2/NM005569.3 | SLCO1A2/NM134431.3 |
| CBX1/NM006807.4 | LIMS1/NM001193485.2 | SLCO1B1/NM006446.4 |
| CBX2/NM005189.2 | LIMS2/NM001161403.1 | SLCO1B7/NM001009562.4 |
| CBX3/NM007276.4 | LIMS3/NM033514.4 | SLCO2A1/NM005630.2 |
| CBX3/NM016587.3 | LIN28A/NM024674.5 | SLCO2B1/NM007256.4 |
| CBX4/NM003655.2 | LIN28B/NM001004317.3 | SLCO3A1/NM013272.3 |
| CBX5/NM001127321.1 | LIN37/NM019104.2 | SLCO4A1/NM016354.3 |
| CBX5/NM001127322.1 | LIN52/NM001024674.2 | SLCO4C1/NM180991.4 |
| CBX5/NM012117.2 | LIN54/NM194282.3 | SLCO5A1/NM030958.2 |
| CBX6/NM014292.4 | LIN7A/NM004664.3 | SLCO6A1/NM173488.4 |
| CBX7/NM175709.3 | LIN7B/NM022165.2 | SLF1/NM032290.3 |
| CBX8/NM020649.2 | LIN7C/NM018362.3 | SLF2/NM018121.3 |
| CBY3/NM001164444.1 | LIN9/NM173083.3 | SLFN11/NM001104587.1 |
| CC2D1A/NM017721.4 | LINC00452/NM001278674.1 | SLFN12/NM001289009.1 |
| CC2D1B/NM032449.2 | LINC01272/NM001278655.1 | SLFN12/NM018042.4 |
| CC2D2A/NM001080522.2 | LINGO1/NM032808.6 | SLFN12L/NM001195790.1 |
| CC2D2B/NM001001732.3 | LINGO2/NM001258282.1 | SLFN13/NM144682.5 |
| CCAR1/NM018237.3 | LINGO2/NM152570.2 | SLFN14/NM001129820.1 |
| CCBE1/NM133459.3 | LINGO3/NM001101391.1 | SLFN5/NM144975.3 |
| CCDC102A/NM033212.3 | LINGO4/NM001004432.3 | SLFNL1/NM001168247.2 |
| CCDC102B/NM001093729.1 | LINS1/NM001040616.2 | SLIT1/NM003061.2 |
| CCDC102B/NM024781.2 | LIPA/NM000235.3 | SLIT2/NM004787.3 |
| CCDC103/NM001258395.1 | LIPA/NM001127605.2 | SLIT3/NM003062.3 |
| CCDC105/NM173482.2 | LIPC/NM000236.2 | SLITRK1/NM052910.2 |
| CCDC106/NM013301.2 | LIPE/NM005357.3 | SLITRK5/NM015567.1 |
| CCDC107/NM174923.2 | LIPG/NM006033.3 | SLITRK6/NM032229.2 |
| CCDC109B/NM017918.4 | LIPH/NM139248.2 | SLK/NM014720.3 |
| CCDC110/NM152775.3 | LIPI/NM198996.3 | SLMAP/NM007159.4 |
| CCDC112/NM152549.2 | LIPJ/NM001010939.2 | SLPI/NM003064.3 |
| CCDC113/NM014157.3 | LIPK/NM001080518.1 | SLU7/NM006425.4 |
| CCDC114/NM144577.3 | LIPM/NM001128215.1 | SLURP1/NM020427.2 |
| CCDC115/NM032357.3 | LIPN/NM001102469.1 | SLX1A/NM001014999.2 |
| CCDC116/NM152612.2 | LIPT1/NM001204830.1 | SLX1B/NM024044.3 |
| CCDC117/NM173510.3 | LIPT1/NM145199.2 | SLX4/NM032444.2 |
| CCDC12/NM001277074.1 | LIPT2/NM001144869.1 | SLX4IP/NM001009608.2 |
| CCDC121/NM024584.4 | LITAF/NM001136472.1 | SMAD1/NM001003688.1 |
| CCDC122/NM144974.3 | LITAF/NM004862.3 | SMAD1/NM005900.2 |
| CCDC124/NM001136203.1 | LIX1/NM153234.4 | SMAD2/NM001003652.3 |
| CCDC124/NM138442.3 | LIX1L/NM153713.2 | SMAD2/NM005901.5 |
| CCDC125/NM176816.4 | LKAAEAR1/NM001007125.1 | SMAD3/NM005902.3 |
| CCDC126/NM138771.3 | LLGL2/NM004524.2 | SMAD4/NM005359.5 |
| CCDC127/NM145265.2 | LLPH/NM032338.3 | SMAD5/NM001001420.2 |
| CCDC129/NM194300.3 | LMAN1/NM005570.3 | SMAD6/NM005585.4 |
| CCDC13/NM144719.3 | LMAN1L/NM021819.2 | SMAD7/NM005904.3 |
| CCDC130/NM030818.3 | LMAN2/NM006816.2 | SMAD9/NM005905.5 |
| CCDC134/NM024821.3 | LMBR1/NM022458.3 | SMAGP/NM001031628.1 |
| CCDC136/NM022742.4 | LMBR1L/NM018113.3 | SMAGP/NM001033873.1 |
| CCDC137/NM199287.2 | LMBRD1/NM018368.3 | SMAP1/NM021940.4 |
| CCDC138/NM144978.2 | LMBRD2/NM001007527.1 | SMAP2/NM022733.2 |
| CCDC14/NM022757.4 | LMCD1/NM014583.3 | SMARCA2/NM001289396.1 |
| CCDC141/NM173648.3 | LMF1/NM022773.2 | SMARCA2/NM003070.4 |
| CCDC142/NM032779.3 | LMF2/NM033200.2 | SMARCA4/NM003072.3 |
| CCDC144A/NM014695.2 | LMLN/NM001136049.2 | SMARCA5/NM003601.3 |
| CCDC144NL/NM001004306.2 | LMNA/NM170707.3 | SMARCAD1/NM020159.4 |
| CCDC146/NM020879.2 | LMNB1/NM005573.3 | SMARCAL1/NM001127207.1 |
| CCDC148/NM138803.3 | LMNB2/NM032737.3 | SMARCAL1/NM014140.3 |
| CCDC149/NM173463.4 | LMNTD2/NM173573.2 | SMARCB1/NM003073.4 |
| CCDC15/NM025004.2 | LMO1/NM002315.2 | SMARCC1/NM003074.3 |
| CCDC150/NM001080539.1 | LMO2/NM001142315.1 | SMARCC2/NM003075.3 |
| CCDC151/NM145045.4 | LMO2/NM001142316.1 | SMARCD1/NM003076.4 |
| CCDC152/NM001134848.1 | LMO3/NM001243611.1 | SMARCD2/NM001098426.1 |
| CCDC153/NM001145018.1 | LMO4/NM006769.3 | SMARCD3/NM001003802.1 |
| CCDC154/NM001143980.1 | LMO7/NM005358.5 | SMARCD3/NM003078.3 |
| CCDC155/NM144688.4 | LMO7DN/NM001257995.1 | SMC1B/NM148674.4 |
| CCDC157/NM001017437.3 | LMOD1/NM012134.2 | SMC2/NM001042550.1 |
| CCDC158/NM001042784.1 | LMOD2/NM207163.1 | SMC2/NM006444.2 |
| CCDC159/NM001080503.2 | LMOD3/NM198271.4 | SMC3/NM005445.3 |
| CCDC166/NM001162914.1 | LMTK2/NM014916.3 | SMC4/NM001002800.2 |
| CCDC167/NM138493.2 | LMTK3/NM001080434.1 | SMC4/NM005496.3 |
| CCDC168/NM001146197.1 | LMX1A/NM001174069.1 | SMC5/NM015110.3 |
| CCDC169-SOHLH2/NM001198910.1 | LMX1A/NM177398.3 | SMCHD1/NM015295.2 |
| CCDC17/NM001114938.2 | LMX1B/NM002316.3 | SMCO1/NM001077657.2 |
| CCDC170/NM025059.3 | LNP1/NM001085451.1 | SMCO2/NM001145010.1 |
| CCDC172/NM198515.2 | LNPEP/NM005575.2 | SMCO3/NM001013698.2 |
| CCDC173/NM001085447.1 | LNX1/NM001126328.2 | SMC04/NM020179.2 |
| CCDC174/NM016474.4 | LNX2/NM153371.3 | SMCP/NM030663.2 |
| CCDC175/NM001164399.1 | LOC100128108/NM001321551.1 | SMCR8/NM144775.2 |
| CCDC177/NM001271507.1 | LOC100128988/NM001318196.1 | SMDT1/NM033318.4 |
| CCDC178/NM198995.2 | LOC100129083/NM001256795.1 | SMG1/NM015092.4 |
| CCDC179/NM001195637.1 | LOC100129216/NM001242853.1 | SMG5/NM015327.2 |
| CCDC18/NM001306076.1 | LOC100129307/NM001310140.1 | SMG6/NM017575.4 |
| CCDC180/NM020893.2 | LOC100129697/NM001290330.1 | SMG8/NM018149.6 |
| CCDC181/NM001300968.1 | LOC100129924/NM001242750.1 | SMG9/NM019108.2 |
| CCDC181/NM021179.2 | LOC100129940/NM001292023.1 | SMIM1/NM001163724.2 |
| CCDC182/NM001282544.1 | LOC100130357/NM001242698.1 | SMIM1/NM001288583.1 |
| CCDC183/NM001039374.4 | LOC100130370/NM001272086.1 | SMIM10L1/NM001271592.1 |
| CCDC184/NM001013635.3 | LOC100130451/NM001242575.1 | SMIM11A/NM058182.4 |
| CCDC185/NM152610.2 | LOC100130520/NM001324073.1 | SMIM11B/NM001313692.1 |
| CCDC186/NM018017.3 | LOC100130705/NM001195150.1 | SMIM12/NM001164824.1 |
| CCDC187/NM001291516.1 | LOC100130880/NM001243523.1 | SMIM12/NM001164825.1 |
| CCDC189/NM001014979.2 | LOC100131107/NM001310142.1 | SMIM12/NM001320261.1 |
| CCDC190/NM178550.4 | LOC100131303/NM001282442.1 | SMIM13/NM001135575.1 |
| CCDC191/NM020817.1 | LOC100132146/NM001195442.1 | SMIM14/NM001317896.1 |
| CCDC192/NM001317938.1 | LOC100132813/NM001317989.1 | SMIM14/NM001317897.1 |
| CCDC24/NM152499.2 | LOC100133267/NM001195257.1 | SMIM15/NM001048249.3 |
| CCDC25/NM018246.2 | LOC100134391/NM001278587.1 | SMIM17/NM001193628.1 |
| CCDC28A/NM015439.2 | LOC100144595/NM001256368.1 | SMIM18/NM001206847.1 |
| CCDC28B/NM024296.4 | LOC100287036/NM001242885.1 | SMIM19/NM001135674.1 |
| CCDC3/NM031455.3 | LOC100287896/NM001319240.1 | SMIM19/NM001135675.1 |
| CCDC30/NM001080850.2 | LOC100289561/NM001242713.1 | SMIM19/NM001135676.1 |
| CCDC33/NM025055.4 | LOC100505478/NM001199233.1 | SMIM19/NM138436.3 |
| CCDC34/NM030771.1 | LOC100505549/NM001242804.1 | SMIM2/NM024058.2 |
| CCDC36/NM001135197.1 | LOC100506127/NM001305225.1 | SMIM20/NM001145432.1 |
| CCDC36/NM178173.3 | LOC100506388/NM001242780.1 | SMIM21/NM001037331.3 |
| CCDC38/NM182496.2 | LOC100506422/NM001004352.2 | SMIM22/NM001253791.1 |
| CCDC39/NM181426.1 | LOC100507507/NM001322263.1 | SMIM22/NM001253794.1 |
| CCDC40/NM017950.3 | LOC100996634/NM001277339.1 | SMIM23/NM001289970.1 |
| CCDC42/NM144681.2 | LOC100996693/NM001286811.1 | SMIM24/NM001136503.1 |
| CCDC43/NM144609.2 | LOC100996842/NM001322800.1 | SMIM3/NM032947.4 |
| CCDC47/NM020198.2 | LOC101060389/NM001291465.1 | SMIM4/NM001124767.1 |
| CCDC50/NM178335.2 | LOC101927322/NM001289967.1 | SMIM5/NM001162995.2 |
| CCDC51/NM001256964.1 | LOC101927503/NM001323425.1 | SMIM6/NM001162997.1 |
| CCDC53/NM016053.3 | LOC101927572/NM001290056.1 | SMIM7/NM024104.3 |
| CCDC54/NM032600.2 | LOC101927844/NM001290053.1 | SMIM8/NM020425.5 |
| CCDC57/NM198082.2 | LOC101928093/NM001287812.1 | SMKR1/NM001195243.1 |
| CCDC58/NM001017928.3 | LOC101928120/NM001321726.1 | SMLR1/NM001195597.1 |
| CCDC59/NM014167.4 | LOC101928436/NM001286997.1 | SMN1/NM000344.3 |
| CCDC6/NM005436.4 | LOC101928841/NM001304433.1 | SMN2/NM017411.3 |
| CCDC61/NM001267723.1 | LOC101929372/NM001288707.1 | SMNDC1/NM005871.3 |
| CCDC62/NM201435.4 | LOC101929726/NM001315494.1 | SMO/NM005631.4 |
| CCDC65/NM033124.4 | LOC101929926/NM001322244.1 | SMOC2/NM022138.2 |
| CCDC66/NM001012506.4 | LOC102723360/NM001322975.1 | SMOX/NM175839.2 |
| CCDC67/NM181645.3 | LOC102724219/NM001322044.1 | SMPD1/NM001007593.2 |
| CCDC68/NM001143829.1 | LOC102724265/NM001319657.1 | SMPD3/NM018667.3 |
| CCDC68/NM025214.2 | LOC102724279/NM001302493.1 | SMPD4/NM017951.4 |
| CCDC69/NM015621.2 | LOC102724428/NM001320643.1 | SMPDL3A/NM006714.4 |
| CCDC7/NM001026383.2 | LOC102724652/NM001314050.2 | SMPDL3B/NM014474.3 |
| CCDC70/NM031290.2 | LOC102724843/NM001322047.1 | SMR3A/NM012390.3 |
| CCDC71/NM022903.3 | LOC102724951/NM001322036.1 | SMR3B/NM006685.3 |
| CCDC71L/NM175884.4 | LOC102724957/NM001293172.1 | SMTN/NM134270.2 |
| CCDC73/NM001008391.3 | LOC105372280/NM001316692.1 | SMTNL1/NM001105565.2 |
| CCDC74A/NM138770.2 | LOC105372977/NM001318392.1 | SMU1/NM018225.2 |
| CCDC77/NM032358.3 | LOC105374250/NM001322103.1 | SMUG1/NM014311.2 |
| CCDC78/NM001031737.2 | LOC105375396/NM001317905.1 | SMURF1/NM181349.2 |
| CCDC8/NM032040.4 | LOC105375787/NM001317971.1 | SMURF2/NM022739.3 |
| CCDC80/NM199511.2 | LOC105376731/NM001321990.1 | SMYD2/NM020197.2 |
| CCDC81/NM001156474.1 | LOC113230/NM001291291.1 | SMYD3/NM001167740.1 |
| CCDC82/NM024725.3 | LOC149373/NM001256615.1 | SMYD4/NM052928.2 |
| CCDC84/NM198489.2 | LOC150051/NM001321023.1 | SMYD5/NM006062.2 |
| CCDC85A/NM001080433.1 | LOC200726/NM001102659.1 | SNAI1/NM005985.3 |
| CCDC85B/NM006848.2 | LOC283710/NM001243538.1 | SNAI2/NM003068.4 |
| CCDC85C/NM001144995.1 | LOC284898/NM001318329.1 | SNAI3/NM178310.3 |
| CCDC86/NM024098.3 | LOC286238/NM001100111.1 | SNAP23/NM003825.3 |
| CCDC87/NM018219.2 | LOC339862/NM001257177.1 | SNAP25/NM003081.4 |
| CCDC88A/NM018084.4 | LOC388282/NM001278081.1 | SNAP29/NM004782.3 |
| CCDC88B/NM032251.5 | LOC388436/NM001291905.1 | SNAP47/NM053052.3 |
| CCDC88C/NM001080414.3 | LOC388780/NM001287682.1 | SNAPC1/NM003082.3 |
| CCDC89/NM152723.2 | LOC388813/NM001256579.2 | SNAPC2/NM003083.3 |
| CCDC9/NM015603.2 | LOC389199/NM203423.2 | SNAPC3/NM001039697.1 |
| CCDC90B/NM021825.4 | LOC389602/NM001291913.1 | SNAPC4/NM003086.2 |
| CCDC91/NM018318.3 | LOC400927-CSNK1E/NM001289912.1 | SNAPC5/NM006049.2 |
| CCDC92/NM001304961.1 | LOC401052/NM001008737.1 | SNAPIN/NM012437.5 |
| CCDC93/NM019044.4 | LOC403312/NM001301851.1 | SNCA/NM000345.3 |
| CCDC94/NM018074.4 | LOC441155/NM001271675.1 | SNCA/NM001146054.1 |
| CCDC96/NM153376.2 | LOC613037/NM001321892.1 | SNCA/NM001146055.1 |
| CCDC97/NM052848.1 | LOC643355/NM001270945.1 | SNCAIP/NM005460.3 |
| CCER1/NM152638.3 | LOC643802/NM001207030.1 | SNCG/NM003087.2 |
| CCER2/NM001243212.1 | LOC645177/NM001321724.1 | SND1/NM014390.2 |
| CCIN/NM005893.2 | LOC650293/NM001040071.1 | SNED1/NM001080437.1 |
| CCKAR/NM000730.2 | LOC653602/NM001291410.1 | SNF8/NM007241.3 |
| CCL1/NM002981.2 | LOC728392/NM001162371.2 | SNIP1/NM024700.3 |
| CCL11/NM002986.2 | LOC728485/NM001294306.1 | SNN/NM003498.5 |
| CCL13/NM005408.2 | LOC729159/NM001282301.1 | SNPH/NM014723.3 |
| CCL14/NM032963.3 | LOC730098/NM001320038.1 | SNRK/NM001100594.1 |
| CCL15/NM032965.5 | LOC730159/NM001195190.1 | SNRNP200/NM014014.4 |
| CCL16/NM004590.3 | LOC730183/NM001256932.1 | SNRNP25/NM024571.3 |
| CCL17/NM002987.2 | LOC79999/NM001291904.1 | SNRNP27/NM006857.2 |
| CCL18/NM002988.3 | LOC81691/NM030941.2 | SNRNP35/NM022717.3 |
| CCL19/NM006274.2 | LONP1/NM004793.3 | SNRNP40/NM004814.2 |
| CCL2/NM002982.3 | LONRF1/NM152271.3 | SNRNP48/NM152551.3 |
| CCL20/NM004591.2 | LONRF2/NM198461.3 | SNRPA/NM004596.4 |
| CCL21/NM002989.3 | LOR/NM000427.2 | SNRPB/NM003091.3 |
| CCL22/NM002990.4 | LOX/NM002317.6 | SNRPB2/NM198220.2 |
| CCL23/NM005064.5 | LOXHD1/NM144612.6 | SNRPC/NM003093.2 |
| CCL24/NM002991.2 | LOXL1/NM005576.3 | SNRPD2/NM004597.6 |
| CCL25/NM005624.3 | LOXL2/NM002318.2 | SNRPD3/NM001278656.1 |
| CCL26/NM006072.4 | LOXL3/NM032603.3 | SNRPE/NM003094.3 |
| CCL27/NM006664.3 | LOXL4/NM032211.6 | SNRPG/NM003096.3 |
| CCL28/NM001301873.1 | LPA/NM005577.2 | SNRPN/NM022805.3 |
| CCL3/NM002983.2 | LPAR1/NM057159.2 | SNRPN/NM022806.3 |
| CCL3L3/NM001001437.3 | LPAR2/NM004720.5 | SNTA1/NM003098.2 |
| CCL4/NM002984.3 | LPAR3/NM012152.2 | SNTB1/NM021021.3 |
| CCL4L2/NM001291468.1 | LPAR5/NM001142961.1 | SNTB2/NM006750.3 |
| CCL5/NM002985.2 | LPAR5/NM020400.5 | SNTG1/NM001287813.2 |
| CCL7/NM006273.3 | LPAR6/NM001162497.1 | SNTG1/NM001321773.1 |
| CCL8/NM005623.2 | LPAR6/NM001162498.1 | SNTG2/NM018968.3 |
| CCM2/NM031443.3 | LPAR6/NM005767.5 | SNU13/NM005008.3 |
| CCNA1/NM001111047.1 | LPCAT1/NM024830.3 | SNUPN/NM001042588.1 |
| CCNA2/NM001237.3 | LPCAT2/NM017839.4 | SNUPN/NM005701.3 |
| CCNB1/NM031966.3 | LPCAT3/NM005768.5 | SNURF/NM005678.4 |
| CCNB1IP1/NM182852.3 | LPCAT4/NM153613.2 | SNURF/NM022804.2 |
| CCNB2/NM004701.3 | LPIN1/NM145693.2 | SNX1/NM003099.4 |
| CCNC/NM005190.3 | LPIN2/NM014646.2 | SNX10/NM013322.2 |
| CCND1/NM053056.2 | LPIN3/NM001301860.1 | SNX11/NM013323.2 |
| CCND2/NM001759.3 | LPL/NM000237.2 | SNX11/NM152244.1 |
| CCND3/NM001760.4 | LPO/NM006151.2 | SNX13/NM015132.4 |
| CCNDBP1/NM012142.4 | LPP/NM001167671.2 | SNX14/NM153816.5 |
| CCNE1/NM001238.3 | LPP/NM005578.4 | SNX15/NM013306.4 |
| CCNE2/NM057749.2 | LPXN/NM004811.2 | SNX16/NM022133.3 |
| CCNF/NM001761.2 | LRAT/NM001301645.1 | SNX17/NM014748.3 |
| CCNG1/NM199246.1 | LRAT/NM004744.4 | SNX18/NM001102575.1 |
| CCNH/NM001239.3 | LRBA/NM006726.4 | SNX19/NM014758.2 |
| CCNI/NM006835.2 | LRCH1/NM015116.2 | SNX2/NM003100.3 |
| CCNI2/NM001039780.3 | LRCH4/NM002319.4 | SNX22/NM024798.2 |
| CCNJ/NM001134375.1 | LRCOL1/NM001195520.1 | SNX24/NM014035.2 |
| CCNJL/NM001308173.1 | LRFN1/NM020862.1 | SNX25/NM001317781.1 |
| CCNK/NM001099402.1 | LRFN2/NM020737.2 | SNX29/NM032167.4 |
| CCNL1/NM020307.3 | LRFN3/NM024509.1 | SNX3/NM003795.5 |
| CCNL2/NM030937.4 | LRFN4/NM024036.4 | SNX30/NM001012994.1 |
| CCNO/NM021147.4 | LRG1/NM052972.2 | SNX32/NM152760.2 |
| CCNT1/NM001240.3 | LRGUK/NM144648.1 | SNX33/NM153271.1 |
| CCNT2/NM058241.2 | LRIF1/NM018372.3 | SNX4/NM003794.3 |
| CCNY/NM145012.5 | LRIG1/NM015541.2 | SNX5/NM014426.3 |
| CCP110/NM014711.5 | LRIG2/NM014813.2 | SNX5/NM152227.2 |
| CCPG1/NM004748.4 | LRIG3/NM153377.4 | SNX7/NM015976.4 |
| CCPG1/NM020739.3 | LRIT1/NM015613.2 | SNX8/NM013321.3 |
| CCR1/NM001295.2 | LRIT2/NM001017924.3 | SNX9/NM016224.4 |
| CCR10/NM016602.2 | LRIT3/NM198506.4 | SOAT1/NM003101.5 |
| CCR2/NM001123396.1 | LRMP/NM001204126.1 | SOAT2/NM003578.3 |
| CCR3/NM178329.2 | LRP1/NM002332.2 | SOBP/NM018013.3 |
| CCR4/NM005508.4 | LRP10/NM014045.3 | SOCS1/NM003745.1 |
| CCR5/NM001100168.1 | LRP11/NM032832.5 | SOCS2/NM001270467.1 |
| CCR6/NM004367.5 | LRP12/NM013437.4 | SOCS2/NM001270468.1 |
| CCR6/NM031409.3 | LRP1B/NM018557.2 | SOCS2/NM001270469.1 |
| CCR7/NM001838.3 | LRP2BP/NM018409.3 | SOCS2/NM001270471.1 |
| CCR8/NM005201.3 | LRP3/NM002333.3 | SOCS2/NM003877.4 |
| CCR9/NM031200.2 | LRP4/NM002334.3 | SOCS3/NM003955.4 |
| CCRL2/NM003965.4 | LRP5/NM002335.3 | SOCS4/NM080867.2 |
| CCS/NM005125.1 | LRP5L/NM001135772.1 | SOCS5/NM014011.4 |
| CCSAP/NM145257.4 | LRP5L/NM182492.2 | SOCS5/NM144949.2 |
| CCSER1/NM001145065.1 | LRP6/NM002336.2 | SOCS6/NM004232.3 |
| CCSER2/NM018999.3 | LRP8/NM004631.4 | SOCS7/NM014598.3 |
| CCT2/NM006431.2 | LRPAP1/NM002337.3 | SOD1/NM000454.4 |
| CCT3/NM005998.4 | LRPPRC/NM133259.3 | SOD2/NM000636.3 |
| CCT4/NM006430.3 | LRR1/NM152329.3 | SOD3/NM003102.2 |
| CCT5/NM012073.4 | LRRC1/NM018214.4 | SOGA1/NM199181.2 |
| CCT6A/NM001762.3 | LRRC10/NM201550.3 | SOGA3/NM001012279.2 |
| CCT6B/NM006584.3 | LRRC10B/NM001145077.1 | SOHLH1/NM001012415.2 |
| CCT7/NM006429.3 | LRRC14/NM014665.3 | SOHLH2/NM017826.2 |
| CCT8/NM006585.3 | LRRC14B/NM001080478.1 | SON/NM138927.2 |
| CCT8L2/NM014406.4 | LRRC16A/NM017640.5 | SORBS1/NM006434.2 |
| CCZ1/NM015622.5 | LRRC17/NM001031692.2 | SORBS2/NM003603.6 |
| CCZ1B/NM198097.3 | LRRC18/NM001006939.3 | SORBS3/NM005775.4 |
| CD101/NM001256109.2 | LRRC19/NM022901.2 | SORCS1/NM052918.4 |
| CD109/NM133493.4 | LRRC2/NM024512.4 | SORCS2/NM020777.2 |
| CD14/NM000591.3 | LRRC20/NM001278211.1 | SORCS3/NM014978.2 |
| CD14/NM001174104.1 | LRRC20/NM207119.2 | SORL1/NM003105.5 |
| CD151/NM001039490.1 | LRRC23/NM201650.2 | SORT1/NM002959.6 |
| CD160/NM007053.3 | LRRC24/NM001024678.3 | SOS1/NM005633.3 |
| CD163/NM004244.5 | LRRC25/NM145256.2 | SOS2/NM006939.2 |
| CD163L1/NM174941.5 | LRRC26/NM001013653.2 | SOST/NM025237.2 |
| CD164/NM006016.4 | LRRC27/NM001143757.1 | SOSTDC1/NM015464.2 |
| CD164L2/NM207397.3 | LRRC27/NM030626.2 | SOWAHA/NM175873.5 |
| CD177/NM020406.3 | LRRC28/NM001321675.1 | SOWAHB/NM001029870.1 |
| CD180/NM005582.2 | LRRC28/NM144598.4 | SOWAHC/NM023016.3 |
| CD19/NM001770.5 | LRRC29/NM001004055.1 | SOX1/NM005986.2 |
| CD1A/NM001763.2 | LRRC29/NM012163.2 | SOX10/NM006941.3 |
| CD1B/NM001764.2 | LRRC3/NM030891.4 | SOX11/NM003108.3 |
| CD1C/NM001765.2 | LRRC30/NM001105581.1 | SOX12/NM006943.3 |
| CD1D/NM001766.3 | LRRC31/NM024727.3 | SOX13/NM005686.2 |
| CD1E/NM030893.3 | LRRC32/NM001128922.1 | SOX14/NM004189.3 |
| CD2/NM001767.3 | LRRC32/NM005512.2 | SOX15/NM006942.1 |
| CD200/NM005944.6 | LRRC34/NM001172779.1 | SOX17/NM022454.3 |
| CD200R1/NM138806.3 | LRRC36/NM018296.5 | SOX18/NM018419.2 |
| CD200R1L/NM001008784.2 | LRRC37A/NM014834.4 | SOX2/NM003106.3 |
| CD207/NM015717.4 | LRRC37A2/NM001006607.2 | SOX21/NM007084.3 |
| CD209/NM021155.3 | LRRC37A3/NM199340.3 | SOX30/NM178424.1 |
| CD22/NM001771.3 | LRRC37B/NM052888.2 | SOX4/NM003107.2 |
| CD226/NM001303618.1 | LRRC38/NM001010847.1 | SOX5/NM006940.4 |
| CD226/NM006566.3 | LRRC39/NM001256386.1 | SOX6/NM033326.3 |
| CD24/NM001291737.1 | LRRC3B/NM001317808.1 | SOX7/NM031439.3 |
| CD24/NM001291738.1 | LRRC3B/NM001317809.1 | SOX8/NM014587.4 |
| CD24/NM013230.3 | LRRC3B/NM001317810.1 | SOX9/NM000346.3 |
| CD244/NM016382.3 | LRRC3B/NM001317811.1 | SP1/NM138473.2 |
| CD247/NM198053.2 | LRRC3B/NM052953.3 | SP100/NM001080391.1 |
| CD248/NM020404.2 | LRRC3C/NM001195545.1 | SP110/NM080424.2 |
| CD274/NM014143.3 | LRRC4/NM022143.4 | SP140/NM007237.4 |
| CD276/NM001024736.1 | LRRC41/NM006369.4 | SP140L/NM138402.4 |
| CD28/NM006139.3 | LRRC42/NM052940.4 | SP2/NM003110.5 |
| CD2AP/NM012120.2 | LRRC43/NM001098519.1 | SP3/NM003111.4 |
| CD2BP2/NM001243646.1 | LRRC45/NM144999.3 | SP4/NM003112.3 |
| CD2BP2/NM006110.2 | LRRC46/NM033413.3 | SP5/NM001003845.2 |
| CD300A/NM007261.3 | LRRC47/NM020710.2 | SP6/NM001258248.1 |
| CD300C/NM006678.4 | LRRC49/NM017691.4 | SP6/NM199262.2 |
| CD300E/NM181449.2 | LRRC4B/NM001080457.1 | SP7/NM001173467.2 |
| CD300LB/NM174892.3 | LRRC4C/NM001258419.1 | SP7/NM152860.1 |
| CD300LD/NM001115152.1 | LRRC52/NM001005214.3 | SP8/NM198956.3 |
| CD300LF/NM139018.4 | LRRC55/NM001005210.2 | SP9/NM001145250.1 |
| CD302/NM014880.4 | LRRC56/NM198075.3 | SPA17/NM017425.3 |
| CD320/NM016579.3 | LRRC57/NM153260.2 | SPACA1/NM030960.2 |
| CD33/NM001772.3 | LRRC58/NM001099678.1 | SPACA4/NM133498.2 |
| CD34/NM001025109.1 | LRRC59/NM018509.3 | SPACA6/NM001316972.1 |
| CD36/NM000072.3 | LRRC6/NM012472.5 | SPACA7/NM145248.4 |
| CD36/NM001001547.2 | LRRC61/NM023942.2 | SPACA9/NM018956.4 |
| CD36/NM001127443.1 | LRRC63/NM001282460.1 | SPAG1/NM003114.4 |
| CD36/NM001127444.1 | LRRC66/NM001024611.2 | SPAG1/NM172218.2 |
| CD37/NM001774.2 | LRRC69/NM001129890.1 | SPAG11B/NM058203.2 |
| CD38/NM001775.3 | LRRC70/NM181506.4 | SPAG16/NM024532.4 |
| CD3D/NM000732.4 | LRRC72/NM001195280.1 | SPAG17/NM206996.2 |
| CD3E/NM000733.3 | LRRC73/NM001012974.2 | SPAG4/NM001317931.1 |
| CD3EAP/NM012099.1 | LRRC74A/NM194287.2 | SPAG5/NM006461.3 |
| CD3G/NM000073.2 | LRRC74B/NM001291006.1 | SPAG6/NM012443.3 |
| CD4/NM000616.4 | LRRC75A/NM207387.3 | SPAG7/NM004890.2 |
| CD40/NM001250.5 | LRRC75B/NM207644.2 | SPAG8/NM172312.1 |
| CD46/NM172359.2 | LRRC8A/NM001127244.1 | SPAG9/NM003971.5 |
| CD47/NM001777.3 | LRRC8A/NM001127245.1 | SPAM1/NM003117.4 |
| CD48/NM001778.3 | LRRC8B/NM001134476.1 | SPARC/NM003118.3 |
| CD5/NM014207.3 | LRRC8B/NM015350.2 | SPARCL1/NM001128310.2 |
| CD52/NM001803.2 | LRRC8D/NM001134479.1 | SPAST/NM014946.3 |
| CD53/NM000560.3 | LRRC8D/NM018103.4 | SPATA1/NM001310156.1 |
| CD53/NM001040033.1 | LRRC8E/NM025061.5 | SPATA12/NM181727.1 |
| CD55/NM000574.4 | LRRCC1/NM033402.4 | SPATA13/NM153023.3 |
| CD58/NM001779.2 | LRRD1/NM001161528.1 | SPATA16/NM031955.5 |
| CD59/NM001127223.1 | LRRFIP1/NM001137551.1 | SPATA17/NM138796.3 |
| CD59/NM001127227.1 | LRRFIP2/NM017724.2 | SPATA18/NM145263.3 |
| CD59/NM203330.2 | LRRIQ1/NM001079910.1 | SPATA19/NM174927.2 |
| CD5L/NM005894.2 | LRRIQ3/NM001105659.1 | SPATA2/NM001135773.1 |
| CD6/NM006725.4 | LRRIQ4/NM001080460.1 | SPATA2/NM006038.3 |
| CD68/NM001251.2 | LRRK1/NM024652.4 | SPATA20/NM022827.3 |
| CD69/NM001781.2 | LRRK2/NM198578.3 | SPATA21/NM198546.1 |
| CD70/NM001252.4 | LRRN1/NM020873.6 | SPATA22/NM001170695.1 |
| CD72/NM001782.2 | LRRN2/NM006338.2 | SPATA22/NM001170697.1 |
| CD74/NM001025159.2 | LRRN2/NM201630.1 | SPATA22/NM001170698.1 |
| CD79A/NM001783.3 | LRRN3/NM018334.4 | SPATA22/NM032598.4 |
| CD79B/NM000626.2 | LRRN4/NM152611.4 | SPATA24/NM194296.1 |
| CD80/NM005191.3 | LRRN4CL/NM203422.3 | SPATA25/NM080608.3 |
| CD81/NM004356.3 | LRRTM1/NM178839.4 | SPATA2L/NM152339.3 |
| CD82/NM002231.3 | LRRTM2/NM015564.2 | SPATA3/NM139073.3 |
| CD83/NM004233.3 | LRRTM3/NM178011.4 | SPATA31A1/NM001085452.2 |
| CD84/NM003874.3 | LRRTM4/NM001134745.1 | SPATA31A3/NM001083124.1 |
| CD86/NM175862.4 | LRTM1/NM020678.3 | SPATA31A5/NM001113541.1 |
| CD8A/NM001145873.1 | LRTM2/NM001039029.2 | SPATA31A6/NM001145196.1 |
| CD8A/NM001768.6 | LRWD1/NM152892.2 | SPATA31A7/NM015667.2 |
| CD8B/NM172213.3 | LSAMP/NM002338.4 | SPATA31C1/NM001145124.1 |
| CD9/NM001769.3 | LSG1/NM018385.2 | SPATA31C2/NM001166137.1 |
| CD93/NM012072.3 | LSM1/NM014462.2 | SPATA31D1/NM001001670.2 |
| CDA/NM001785.2 | LSM10/NM032881.2 | SPATA31D3/NM207416.2 |
| CDADC1/NM030911.3 | LSM11/NM173491.3 | SPATA31D4/NM001145197.1 |
| CDAN1/NM138477.2 | LSM12/NM152344.3 | SPATA31E1/NM178828.4 |
| CDC123/NM006023.2 | LSM14A/NM015578.2 | SPATA32/NM152343.2 |
| CDC14A/NM033312.2 | LSM14B/NM144703.2 | SPATA33/NM153025.2 |
| CDC14B/NM001077181.1 | LSM2/NM021177.4 | SPATA45/NM001024601.2 |
| CDC16/NM003903.3 | LSM3/NM014463.2 | SPATA5/NM145207.2 |
| CDC20/NM001255.2 | LSM4/NM012321.4 | SPATA6/NM019073.3 |
| CDC20B/NM152623.2 | LSM6/NM007080.2 | SPATA7/NM018418.4 |
| CDC23/NM004661.3 | LSM7/NM016199.2 | SPATA8/NM173499.4 |
| CDC25A/NM001789.2 | LSM8/NM016200.4 | SPATA9/NM031952.3 |
| CDC25B/NM021873.3 | LSMEM1/NM001134468.1 | SPATC1/NM198572.2 |
| CDC25C/NM001790.4 | LSMEM1/NM182597.2 | SPATC1L/NM001142854.1 |
| CDC26/NM139286.3 | LSMEM2/NM153215.2 | SPATS2/NM023071.3 |
| CDC27/NM001256.4 | LSP1/NM002339.2 | SPATS2L/NM001100422.1 |
| CDC34/NM004359.1 | LSR/NM205834.3 | SPATS2L/NM001100423.1 |
| CDC37L1/NM017913.3 | LSS/NM001001438.2 | SPATS2L/NM015535.2 |
| CDC40/NM015891.2 | LST1/NM205839.2 | SPC24/NM182513.3 |
| CDC42/NM001039802.1 | LTA/NM000595.3 | SPC25/NM020675.3 |
| CDC42BPA/NM003607.3 | LTA/NM001159740.2 | SPCS1/NM014041.3 |
| CDC42BPB/NM006035.3 | LTB/NM002341.1 | SPCS2/NM014752.2 |
| CDC42BPG/NM017525.2 | LTB4R/NM181657.3 | SPCS3/NM021928.3 |
| CDC42EP1/NM152243.2 | LTBP1/NM000627.3 | SPDEF/NM012391.2 |
| CDC42EP2/NM006779.3 | LTBP2/NM000428.2 | SPDL1/NM017785.4 |
| CDC42EP3/NM001270436.1 | LTBP3/NM001130144.2 | SPDYA/NM001142634.1 |
| CDC42EP3/NM006449.4 | LTBP4/NM001042544.1 | SPDYC/NM001008778.1 |
| CDC42EP4/NM012121.4 | LTBR/NM002342.2 | SPDYE1/NM175064.2 |
| CDC42EP5/NM145057.3 | LTC4S/NM145867.1 | SPDYE2/NM001031618.3 |
| CDC42SE1/NM001038707.1 | LTF/NM002343.5 | SPDYE2B/NM001166339.1 |
| CDC42SE2/NM001038702.1 | LTK/NM002344.5 | SPDYE3/NM001004351.4 |
| CDC45/NM003504.4 | LTV1/NM032860.3 | SPDYE4/NM001128076.1 |
| CDC5L/NM001253.3 | LUC7L/NM201412.2 | SPDYE5/NM001306141.1 |
| CDC6/NM001254.3 | LUC7L2/NM016019.4 | SPDYE6/NM001146210.2 |
| CDC7/NM001134419.1 | LUC7L3/NM006107.3 | SPECC1/NM001033553.2 |
| CDC7/NM001134420.1 | LUM/NM002345.3 | SPECC1/NM001243439.1 |
| CDC73/NM024529.4 | LURAP1/NM001013615.2 | SPECC1L/NM001145468.3 |
| CDCA2/NM152562.3 | LURAP1L/NM203403.1 | SPEF1/NM015417.4 |
| CDCA3/NM031299.5 | LUZP1/NM033631.3 | SPEF2/NM024867.3 |
| CDCA5/NM080668.3 | LUZP2/NM001009909.3 | SPEG/NM005876.4 |
| CDCA7L/NM018719.4 | LUZP6/NM001128619.2 | SPEM1/NM199339.2 |
| CDCA8/NM018101.3 | LVRN/NM173800.4 | SPEN/NM015001.2 |
| CDCP1/NM022842.4 | LXN/NM020169.3 | SPG11/NM025137.3 |
| CDCP2/NM201546.3 | LY6D/NM003695.2 | SPG20/NM001142294.1 |
| CDH1/NM004360.4 | LY6G5B/NM021221.2 | SPG20/NM001142295.1 |
| CDH11/NM001797.3 | LY6G5C/NM025262.3 | SPG20/NM015087.4 |
| CDH12/NM001317227.1 | LY6G6C/NM025261.2 | SPG21/NM001127889.4 |
| CDH12/NM004061.4 | LY6G6D/NM021246.2 | SPG21/NM016630.6 |
| CDH13/NM001220488.1 | LY6G6F/NM001003693.1 | SPG7/NM003119.3 |
| CDH15/NM004933.2 | LY6H/NM002347.4 | SPHAR/NM006542.3 |
| CDH16/NM004062.3 | LY6K/NM017527.3 | SPHK1/NM182965.2 |
| CDH17/NM001144663.1 | LY75-CD302/NM001198759.1 | SPHK2/NM001204159.2 |
| CDH17/NM004063.3 | LY86/NM004271.3 | SPHK2/NM020126.4 |
| CDH18/NM001291956.1 | LY9/NM002348.3 | SPHKAP/NM030623.3 |
| CDH19/NM021153.3 | LY96/NM015364.4 | SPIB/NM001243998.1 |
| CDH2/NM001792.4 | LYAR/NM001145725.1 | SPIC/NM152323.1 |
| CDH20/NM031891.3 | LYAR/NM017816.2 | SPICE1/NM144718.3 |
| CDH22/NM021248.2 | LYG1/NM174898.2 | SPIDR/NM001080394.3 |
| CDH23/NM022124.5 | LYG2/NM175735.3 | SPIN1/NM006717.2 |
| CDH24/NM144985.3 | LYL1/NM005583.4 | SPINK1/NM003122.4 |
| CDH26/NM177980.2 | LYN/NM002350.3 | SPINK13/NM001040129.2 |
| CDH3/NM001793.5 | LYNX1/NM177457.4 | SPINK14/NM001001325.2 |
| CDH4/NM001794.4 | LYNX1/NM177476.3 | SPINK2/NM021114.3 |
| CDH5/NM001795.4 | LYNX1/NM177477.3 | SPINK4/NM014471.1 |
| CDH6/NM004932.3 | LYPD1/NM144586.6 | SPINK5/NM006846.3 |
| CDH7/NM004361.3 | LYPD2/NM205545.2 | SPINK7/NM032566.2 |
| CDH7/NM033646.2 | LYPD3/NM014400.2 | SPINK8/NM001080525.1 |
| CDH9/NM016279.3 | LYPD4/NM173506.6 | SPINK9/NM001040433.1 |
| CDHR1/NM033100.3 | LYPD5/NM001288763.1 | SPINT1/NM181642.2 |
| CDHR2/NM001171976.1 | LYPD5/NM182573.2 | SPINT2/NM021102.3 |
| CDHR2/NM017675.4 | LYPD6/NM001195685.1 | SPINT3/NM006652.1 |
| CDHR3/NM152750.4 | LYPD6/NM194317.3 | SPINT4/NM178455.1 |
| CDHR4/NM001007540.3 | LYPD6B/NM001317006.1 | SPIRE1/NM020148.2 |
| CDHR5/NM021924.4 | LYPD8/NM001085474.1 | SPIRE2/NM032451.1 |
| CDIP1/NM001199054.1 | LYPLA1/NM006330.3 | SPN/NM001030288.2 |
| CDIP1/NM013399.2 | LYPLA2/NM007260.2 | SPN/NM003123.4 |
| CDIPT/NM006319.4 | LYRM1/NM001128302.2 | SPNS1/NM001142448.1 |
| CDK1/NM001320918.1 | LYRM1/NM001302835.1 | SPNS2/NM001124758.1 |
| CDK1/NM001786.4 | LYRM1/NM001302836.1 | SPNS3/NM182538.4 |
| CDK10/NM052988.4 | LYRM1/NM020424.4 | SPO11/NM012444.2 |
| CDK11A/NM024011.3 | LYRM2/NM020466.4 | SPOCD1/NM144569.5 |
| CDK11B/NM001787.2 | LYRM4/NM020408.5 | SPOCK1/NM004598.3 |
| CDK12/NM016507.3 | LYRM5/NM001001660.2 | SPOCK2/NM001244950.1 |
| CDK13/NM003718.4 | LYRM7/NM181705.3 | SPOCK2/NM014767.2 |
| CDK14/NM001287135.1 | LYRM9/NM001076680.1 | SPOCK3/NM001040159.1 |
| CDK17/NM002595.4 | LYSMD1/NM212551.4 | SPON1/NM006108.3 |
| CDK18/NM212503.2 | LYSMD2/NM153374.2 | SPON2/NM001128325.2 |
| CDK19/NM015076.4 | LYSMD3/NM198273.2 | SPON2/NM001199021.1 |
| CDK2/NM001798.4 | LYSMD4/NM152449.3 | SPON2/NM012445.3 |
| CDK20/NM001039803.2 | LYST/NM000081.3 | SPOP/NM001007226.1 |
| CDK2AP1/NM004642.3 | LYST/NM001301365.1 | SPOPL/NM001001664.2 |
| CDK2AP2/NM005851.4 | LYVE1/NM006691.3 | SPP1/NM000582.2 |
| CDK3/NM001258.2 | LYZ/NM000239.2 | SPP2/NM006944.2 |
| CDK4/NM000075.3 | LYZL1/NM032517.4 | SPPL2A/NM032802.3 |
| CDK5/NM004935.3 | LYZL2/NM183058.2 | SPPL2B/NM152988.2 |
| CDK5R1/NM003885.2 | LYZL6/NM020426.3 | SPPL2C/NM175882.2 |
| CDK5R2/NM003936.4 | LZTFL1/NM020347.3 | SPR/NM003124.4 |
| CDK5RAP1/NM016408.3 | LZTR1/NM006767.3 | SPRED1/NM152594.2 |
| CDK5RAP2/NM018249.5 | LZTS1/NM021020.3 | SPRED2/NM181784.2 |
| CDK5RAP3/NM176096.2 | LZTS2/NM032429.3 | SPRED3/NM001042522.2 |
| CDK6/NM001145306.1 | M1AP/NM138804.4 | SPRN/NM001012508.3 |
| CDK6/NM001259.6 | M6PR/NM002355.3 | SPRR1A/NM005987.3 |
| CDK7/NM001799.3 | MAATS1/NM033364.3 | SPRR1B/NM003125.2 |
| CDK8/NM001260.2 | MAB21L1/NM005584.4 | SPRR2A/NM005988.2 |
| CDK9/NM001261.3 | MAB21L2/NM006439.4 | SPRR2B/NM001017418.2 |
| CDKAL1/NM017774.3 | MAB21L3/NM152367.2 | SPRR2D/NM006945.4 |
| CDKL1/NM004196.4 | MACC1/NM182762.3 | SPRR2E/NM001024209.3 |
| CDKL2/NM003948.3 | MACROD1/NM014067.3 | SPRR2F/NM001014450.2 |
| CDKN1A/NM000389.4 | MACROD2/NM080676.5 | SPRR2G/NM001014291.3 |
| CDKN1A/NM001220777.1 | MAD1L1/NM003550.2 | SPRR3/NM001097589.1 |
| CDKN1A/NM001220778.1 | MAD2L1/NM002358.3 | SPRR4/NM173080.2 |
| CDKN1A/NM078467.2 | MAD2L1BP/NM014628.2 | SPRTN/NM032018.6 |
| CDKN1B/NM004064.4 | MAD2L2/NM001127325.1 | SPRY1/NM001258039.1 |
| CDKN1C/NM000076.2 | MAD2L2/NM006341.3 | SPRY1/NM005841.2 |
| CDKN2A/NM058195.3 | MADCAM1/NM130760.2 | SPRY1/NM199327.2 |
| CDKN2AIP/NM017632.3 | MADD/NM003682.3 | SPRY2/NM001318536.1 |
| CDKN2AIPNL/NM080656.2 | MAEA/NM001017405.2 | SPRY2/NM001318537.1 |
| CDKN2B/NM004936.3 | MAEL/NM032858.2 | SPRY2/NM005842.3 |
| CDKN2C/NM001262.2 | MAF1/NM032272.4 | SPRY4/NM001127496.1 |
| CDKN2C/NM078626.2 | MAFA/NM201589.3 | SPRY4/NM001293289.1 |
| CDKN2D/NM001800.3 | MAFB/NM005461.4 | SPRYD3/NM032840.2 |
| CDKN2D/NM079421.2 | MAFF/NM001161573.1 | SPRYD4/NM207344.3 |
| CDKN3/NM005192.3 | MAFF/NM012323.3 | SPRYD7/NM020456.3 |
| CDNF/NM001029954.2 | MAFG/NM002359.3 | SPSB1/NM025106.3 |
| CDO1/NM001801.2 | MAFG/NM032711.3 | SPSB2/NM001146316.1 |
| CDON/NM016952.4 | MAFK/NM002360.3 | SPSB2/NM001319670.1 |
| CDPF1/NM207327.4 | MAGEF1/NM022149.4 | SPSB3/NM080861.3 |
| CDR2/NM001802.1 | MAGEL2/NM019066.4 | SPSB4/NM080862.2 |
| CDR2L/NM014603.2 | MAGI1/NM004742.2 | SPTA1/NM003126.2 |
| CDRT1/NM006382.3 | MAGI2/NM012301.3 | SPTAN1/NM001130438.2 |
| CDRT15/NM001007530.1 | MAGI3/NM152900.2 | SPTB/NM001024858.2 |
| CDRT15L2/NM001190790.1 | MAGOH/NM002370.3 | SPTBN1/NM003128.2 |
| CDS1/NM001263.3 | MAGOHB/NM018048.4 | SPTBN2/NM006946.2 |
| CDSN/NM001264.4 | MAK/NM005906.5 | SPTBN4/NM020971.2 |
| CDT1/NM030928.3 | MAK16/NM032509.3 | SPTBN5/NM016642.3 |
| CDV3/NM017548.4 | MAL/NM002371.3 | SPTLC1/NM006415.3 |
| CDX1/NM001804.2 | MAL2/NM052886.2 | SPTLC2/NM004863.3 |
| CDX2/NM001265.4 | MALL/NM005434.4 | SPTLC3/NM018327.2 |
| CDYL/NM004824.3 | MALRD1/NM001142308.2 | SPTSSA/NM138288.3 |
| CDYL2/NM152342.3 | MALSU1/NM138446.1 | SPTSSB/NM001040100.1 |
| CEACAM1/NM001712.4 | MALT1/NM006785.3 | SPTSSB/NM001320679.1 |
| CEACAM16/NM001039213.3 | MAMDC2/NM153267.4 | SPTY2D1/NM194285.2 |
| CEACAM18/NM001278392.1 | MAMDC4/NM206920.2 | SPX/NM030572.3 |
| CEACAM19/NM020219.3 | MAML1/NM014757.4 | SPZ1/NM032567.3 |
| CEACAM20/NM001102597.2 | MAML2/NM032427.3 | SQLE/NM003129.3 |
| CEACAM21/NM001098506.3 | MAML3/NM018717.4 | SQRDL/NM001271213.1 |
| CEACAM3/NM001815.4 | MAMSTR/NM182574.2 | SQRDL/NM021199.3 |
| CEACAM4/NM001817.3 | MAN1A1/NM005907.3 | SQSTM1/NM003900.4 |
| CEACAM5/NM001291484.2 | MAN1B1/NM016219.4 | SRA1/NM001035235.3 |
| CEACAM6/NM002483.6 | MAN1C1/NM020379.3 | SRBD1/NM018079.4 |
| CEACAM7/NM006890.4 | MAN2A1/NM002372.3 | SRC/NM005417.4 |
| CEACAM8/NM001816.3 | MAN2A2/NM006122.3 | SRC/NM198291.2 |
| CEBPA/NM004364.4 | MAN2B1/NM000528.3 | SRCAP/NM006662.2 |
| CEBPB/NM005194.3 | MAN2C1/NM006715.3 | SRCIN1/NM025248.2 |
| CEBPD/NM005195.3 | MANBA/NM005908.3 | SRD5A1/NM001047.3 |
| CEBPE/NM001805.3 | MANBAL/NM001003897.1 | SRD5A2/NM000348.3 |
| CEBPG/NM001252296.1 | MANEA/NM024641.3 | SRD5A3/NM024592.4 |
| CEBPG/NM001806.3 | MANEAL/NM001113482.1 | SREBF1/NM004176.4 |
| CEBPZ/NM005760.2 | MANF/NM006010.5 | SREK1/NM001077199.2 |
| CEBPZOS/NM001322375.1 | MANSC1/NM018050.2 | SREK1IP1/NM173829.3 |
| CECR1/NM001282225.1 | MANSC4/NM001146221.1 | SRF/NM003131.3 |
| CECR2/NM001290046.1 | MAP10/NM019090.2 | SRGAP1/NM020762.2 |
| CECR5/NM017829.5 | MAP1A/NM002373.5 | SRGAP2/NM015326.4 |
| CECR6/NM031890.3 | MAP1B/NM005909.4 | SRGAP2B/NM001271870.2 |
| CEL/NM001807.4 | MAP1LC3A/NM032514.3 | SRGAP2C/NM001271872.1 |
| CELA1/NM001971.5 | MAP1LC3B/NM022818.4 | SRGAP3/NM014850.3 |
| CELA2A/NM033440.2 | MAP1LC3B2/NM001085481.1 | SRGN/NM001321053.1 |
| CELA2B/NM015849.2 | MAP1LC3C/NM001004343.2 | SRGN/NM002727.3 |
| CELF1/NM001172639.1 | MAP2K1/NM002755.3 | SRI/NM003130.3 |
| CELF2/NM001025076.2 | MAP2K2/NM030662.3 | SRL/NM001098814.1 |
| CELF3/NM007185.6 | MAP2K3/NM145109.2 | SRM/NM003132.2 |
| CELF5/NM021938.3 | MAP2K4/NM003010.3 | SRMS/NM080823.3 |
| CELF6/NM052840.4 | MAP2K5/NM145160.2 | SRP14/NM003134.5 |
| CELSR1/NM014246.1 | MAP2K6/NM002758.3 | SRP19/NM003135.2 |
| CELSR2/NM001408.2 | MAP2K7/NM145185.3 | SRP54/NM003136.3 |
| CELSR3/NM001407.2 | MAP3K1/NM005921.1 | SRP68/NM014230.3 |
| CEMIP/NM018689.2 | MAP3K10/NM002446.3 | SRP72/NM006947.3 |
| CEMP1/NM001048212.3 | MAP3K11/NM002419.3 | SRP9/NM003133.5 |
| CEND1/NM016564.3 | MAP3K13/NM001242314.1 | SRPK2/NM182692.2 |
| CENPA/NM001809.3 | MAP3K13/NM004721.4 | SRPRA/NM003139.3 |
| CENPB/NM001810.5 | MAP3K14/NM003954.4 | SRPRB/NM021203.3 |
| CENPBD1/NM145039.3 | MAP3K2/NM006609.4 | SRR/NM021947.2 |
| CENPC/NM001812.2 | MAP3K3/NM203351.1 | SRRD/NM001013694.2 |
| CENPE/NM001813.2 | MAP3K4/NM005922.3 | SRRM1/NM005839.3 |
| CENPF/NM016343.3 | MAP3K5/NM005923.3 | SRRM2/NM016333.3 |
| CENPH/NM022909.3 | MAP3K6/NM004672.4 | SRRM4/NM194286.3 |
| CENPJ/NM018451.4 | MAP3K7/NM145331.2 | SRRM5/NM001145641.1 |
| CENPL/NM001171182.1 | MAP3K7CL/NM001286618.1 | SRRT/NM015908.5 |
| CENPL/NM033319.3 | MAP3K7CL/NM001286619.1 | SRSF1/NM006924.4 |
| CENPM/NM024053.4 | MAP3K7CL/NM001286620.1 | SRSF10/NM054016.3 |
| CENPN/NM001100624.2 | MAP3K8/NM001244134.1 | SRSF12/NM080743.4 |
| CENPP/NM001012267.2 | MAP3K8/NM001320961.1 | SRSF2/NM003016.4 |
| CENPQ/NM018132.3 | MAP3K9/NM033141.3 | SRSF3/NM003017.4 |
| CENPT/NM025082.3 | MAP4/NM002375.4 | SRSF4/NM005626.4 |
| CENPU/NM024629.3 | MAP4K2/NM004579.4 | SRSF5/NM001039465.1 |
| CENPV/NM181716.2 | MAP4K3/NM003618.3 | SRSF5/NM006925.4 |
| CENPW/NM001012507.3 | MAP4K4/NM001242559.1 | SRSF6/NM006275.5 |
| CEP104/NM014704.3 | MAP4K5/NM198794.2 | SRSF7/NM001031684.2 |
| CEP112/NM001199165.2 | MAP6D1/NM024871.2 | SRSF8/NM032102.3 |
| CEP120/NM153223.3 | MAP7/NM003980.4 | SRSF9/NM003769.2 |
| CEP126/NM020802.3 | MAP7D1/NM018067.4 | SRXN1/NM080725.2 |
| CEP128/NM152446.3 | MAP9/NM001039580.1 | SS18/NM001007559.2 |
| CEP131/NM001009811.3 | MAPK1/NM002745.4 | SS18L1/NM198935.2 |
| CEP135/NM025009.4 | MAPK11/NM002751.6 | SS18L2/NM016305.3 |
| CEP152/NM014985.3 | MAPK12/NM002969.4 | SSB/NM001294145.1 |
| CEP162/NM014895.3 | MAPK13/NM002754.4 | SSB/NM003142.4 |
| CEP164/NM014956.4 | MAPK14/NM001315.2 | SSBP1/NM001256510.1 |
| CEP170/NM014812.2 | MAPK15/NM139021.2 | SSBP1/NM003143.2 |
| CEP19/NM032898.4 | MAPK1IP1L/NM144578.3 | SSBP2/NM012446.3 |
| CEP192/NM032142.3 | MAPK3/NM002746.2 | SSBP3/NM145716.3 |
| CEP250/NM007186.5 | MAPK4/NM002747.3 | SSBP4/NM032627.4 |
| CEP290/NM025114.3 | MAPK6/NM002748.3 | SSC4D/NM080744.1 |
| CEP295/NM033395.1 | MAPK7/NM139033.2 | SSC5D/NM001144950.1 |
| CEP295NL/NM001243540.1 | MAPK7/NM139034.2 | SSFA2/NM006751.6 |
| CEP350/NM014810.4 | MAPK8/NM001278547.1 | SSH1/NM018984.3 |
| CEP41/NM018718.2 | MAPK8IP1/NM005456.3 | SSH2/NM033389.3 |
| CEP44/NM001040157.2 | MAPK8IP2/NM012324.4 | SSH3/NM017857.3 |
| CEP55/NM001127182.1 | MAPK8IP3/NM015133.4 | SSMEM1/NM145268.3 |
| CEP55/NM018131.4 | MAPK9/NM002752.4 | SSNA1/NM003731.2 |
| CEP57/NM014679.4 | MAPKAP1/NM024117.3 | SSPN/NM005086.4 |
| CEP57L1/NM001271852.1 | MAPKAPK2/NM004759.4 | SSPO/NM198455.2 |
| CEP57L1/NM173830.4 | MAPKAPK3/NM001243926.1 | SSR1/NM003144.4 |
| CEP63/NM025180.3 | MAPKAPK3/NM004635.4 | SSR2/NM003145.3 |
| CEP68/NM015147.2 | MAPKAPK5/NM139078.2 | SSR3/NM007107.4 |
| CEP70/NM024491.3 | MAPKBP1/NM014994.2 | SSRP1/NM003146.2 |
| CEP72/NM018140.3 | MAPRE1/NM012325.2 | SSSCA1/NM006396.2 |
| CEP76/NM024899.3 | MAPRE2/NM014268.3 | SST/NM001048.3 |
| CEP78/NM001098802.1 | MAPRE3/NM001303050.1 | SSTR1/NM001049.2 |
| CEP83/NM016122.2 | MAPRE3/NM012326.3 | SSTR2/NM001050.2 |
| CEP85/NM022778.4 | MAPT/NM016835.4 | SSTR3/NM001051.4 |
| CEP85L/NM001042475.2 | MARC1/NM022746.3 | SSTR3/NM001278687.2 |
| CEP97/NM024548.3 | MARC2/NM017898.4 | SSTR4/NM001052.2 |
| CEPT1/NM001007794.1 | MARCH1/NM001166373.1 | SSTR5/NM001053.3 |
| CEPT1/NM006090.3 | MARCH10/NM001100875.2 | SSTR5/NM001172560.1 |
| CER1/NM005454.2 | MARCH10/NM152598.3 | SSU72/NM014188.2 |
| CERCAM/NM016174.4 | MARCH11/NM001102562.1 | SSUH2/NM001256748.1 |
| CERK/NM022766.5 | MARCH2/NM001005415.1 | SSX2IP/NM001166417.1 |
| CERKL/NM201548.4 | MARCH3/NM178450.4 | SSX2IP/NM014021.3 |
| CERS2/NM181746.3 | MARCH4/NM020814.2 | ST13/NM003932.4 |
| CERS3/NM001290342.2 | MARCH5/NM017824.4 | ST14/NM021978.3 |
| CERS3/NM001290343.1 | MARCH6/NM005885.3 | ST18/NM014682.2 |
| CERS4/NM024552.2 | MARCH7/NM022826.3 | ST20-MTHFS/NM001199760.1 |
| CERS5/NM147190.3 | MARCH8/NM001002266.2 | ST20/NM001100879.1 |
| CERS6/NM203463.2 | MARCH8/NM145021.5 | ST20/NM001100880.2 |
| CES1/NM001025195.1 | MARCH9/NM138396.5 | ST3GAL1/NM003033.3 |
| CES2/NM003869.5 | MARCKS/NM002356.6 | ST3GAL2/NM006927.3 |
| CES3/NM024922.5 | MARCKSL1/NM023009.6 | ST3GAL3/NM006279.3 |
| CES5A/NM145024.2 | MARCO/NM006770.3 | ST3GAL4/NM006278.2 |
| CETN1/NM004066.2 | MARK1/NM018650.4 | ST3GAL5/NM003896.3 |
| CETN3/NM004365.3 | MARK2/NM004954.4 | ST3GAL6/NM001271146.1 |
| CETP/NM000078.2 | MARK3/NM002376.6 | ST3GAL6/NM006100.3 |
| CFAP100/NM182628.2 | MARK4/NM031417.3 | ST5/NM005418.3 |
| CFAP126/NM001013625.3 | MARS/NM004990.3 | ST5/NM213618.1 |
| CFAP157/NM001012502.2 | MARS2/NM138395.3 | ST6GAL1/NM003032.2 |
| CFAP161/NM173528.2 | MARVELD1/NM031484.3 | ST6GAL1/NM173216.2 |
| CFAP20/NM013242.2 | MARVELD2/NM001038603.2 | ST6GAL2/NM001142351.1 |
| CFAP206/NM001031743.2 | MARVELD3/NM052858.5 | ST6GAL2/NM001322362.1 |
| CFAP221/NM001271049.1 | MAS1/NM002377.2 | ST6GAL2/NM032528.2 |
| CFAP36/NM080667.6 | MAS1L/NM052967.1 | ST6GALNAC1/NM018414.4 |
| CFAP43/NM025145.5 | MASP1/NM001879.5 | ST6GALNAC2/NM006456.2 |
| CFAP44/NM018338.3 | MASP2/NM006610.3 | ST6GALNAC3/NM152996.2 |
| CFAP45/NM012337.2 | MAST1/NM014975.2 | ST6GALNAC4/NM175039.3 |
| CFAP46/NM001200049.2 | MAST2/NM015112.2 | ST6GALNAC5/NM030965.2 |
| CFAP52/NM145054.4 | MAST3/NM015016.1 | ST6GALNAC6/NM013443.4 |
| CFAP53/NM145020.4 | MAST4/NM015183.2 | ST7/NM018412.3 |
| CFAP54/NM001306084.1 | MASTL/NM032844.4 | ST8SIA1/NM003034.3 |
| CFAP57/NM001167965.1 | MAT1A/NM000429.2 | ST8SIA2/NM006011.3 |
| CFAP58/NM001008723.1 | MAT2A/NM005911.5 | ST8SIA3/NM015879.2 |
| CFAP61/NM015585.3 | MAT2B/NM013283.4 | ST8SIA4/NM005668.5 |
| CFAP69/NM001039706.2 | MATK/NM139354.2 | ST8SIA5/NM013305.5 |
| CFAP70/NM145170.3 | MATN1/NM002379.3 | ST8SIA6/NM001004470.1 |
| CFAP73/NM001144872.1 | MATN2/NM002380.4 | STAB1/NM015136.2 |
| CFAP74/NM001304360.1 | MATN3/NM002381.4 | STAC/NM003149.2 |
| CFAP77/NM001282957.1 | MATR3/NM001194954.1 | STAC2/NM198993.3 |
| CFAP97/NM020827.2 | MATR3/NM001194955.1 | STAC3/NM145064.2 |
| CFAP99/NM001193282.2 | MATR3/NM018834.5 | STAG1/NM005862.2 |
| CFB/NM001710.5 | MAX/NM002382.4 | STAG3/NM001282716.1 |
| CFC1/NM032545.3 | MAZ/NM001042539.2 | STAG3/NM012447.3 |
| CFC1B/NM001079530.1 | MB/NM005368.2 | STAM/NM003473.3 |
| CFD/NM001928.3 | MB/NM203377.1 | STAM2/NM005843.5 |
| CFDP1/NM006324.2 | MB/NM203378.1 | STAMBP/NM006463.4 |
| CFH/NM000186.3 | MB21D1/NM138441.2 | STAMBP/NM201647.2 |
| CFHR1/NM002113.2 | MB21D2/NM178496.3 | STAMBP/NM213622.2 |
| CFHR2/NM005666.3 | MBD1/NM001204139.1 | STAMBPL1/NM020799.3 |
| CFHR3/NM021023.5 | MBD2/NM003927.4 | STAP1/NM001317769.1 |
| CFHR5/NM030787.3 | MBD3/NM001281453.1 | STAP2/NM017720.2 |
| CFI/NM000204.4 | MBD3L1/NM145208.2 | STAR/NM000349.2 |
| CFL2/NM021914.7 | MBD3L2/NM144614.3 | STARD10/NM006645.2 |
| CFL2/NM138638.4 | MBD3L3/NM001164425.1 | STARD13/NM178006.3 |
| CFLAR/NM001127183.2 | MBD3L4/NM001164419.1 | STARD3/NM006804.3 |
| CFLAR/NM003879.5 | MBD3L5/NM001136507.1 | STARD3NL/NM032016.3 |
| CFTR/NM000492.3 | MBD4/NM003925.2 | STARD4/NM139164.2 |
| CGB1/NM033377.1 | MBIP/NM016586.2 | STARD5/NM181900.2 |
| CGB2/NM033378.1 | MBL2/NM000242.2 | STARD6/NM139171.1 |
| CGB3/NM000737.3 | MBLAC1/NM203397.2 | STARD7/NM020151.3 |
| CGB5/NM033043.1 | MBLAC2/NM203406.1 | STARD9/NM020759.2 |
| CGB7/NM033142.1 | MBNL1/NM207293.1 | STAT2/NM005419.3 |
| CGB8/NM033183.2 | MBNL2/NM144778.3 | STAT3/NM139276.2 |
| CGGBP1/NM001008390.1 | MBOAT1/NM001080480.2 | STAT4/NM001243835.1 |
| CGGBP1/NM001195308.1 | MBOAT2/NM138799.3 | STAT4/NM003151.3 |
| CGN/NM020770.2 | MBOAT4/NM001100916.1 | STAT5A/NM001288718.1 |
| CGNL1/NM032866.4 | MBOAT7/NM024298.4 | STAT5A/NM003152.3 |
| CGREF1/NM001166239.1 | MBP/NM001025090.1 | STAT5B/NM012448.3 |
| CGREF1/NM006569.5 | MBTD1/NM017643.2 | STAT6/NM001178078.1 |
| CGRRF1/NM006568.2 | MBTPS1/NM003791.3 | STAT6/NM001178079.1 |
| CH17-360D5.1/NM001278795.1 | MC1R/NM002386.3 | STATH/NM003154.2 |
| CH25H/NM003956.3 | MC2R/NM000529.2 | STAU1/NM017453.3 |
| CHAC1/NM024111.3 | MC2R/NM001291911.1 | STAU2/NM001164380.1 |
| CHAC2/NM001008708.2 | MC3R/NM019888.3 | STBD1/NM003943.4 |
| CHAD/NM001267.2 | MC4R/NM005912.2 | STC1/NM003155.2 |
| CHADL/NM138481.1 | MC5R/NM005913.2 | STC2/NM003714.2 |
| CHAF1A/NM005483.2 | MCAM/NM006500.2 | STEAP1/NM012449.2 |
| CHAF1B/NM005441.2 | MCAT/NM173467.4 | STEAP1B/NM207342.2 |
| CHAMP1/NM001164145.1 | MCC/NM001085377.1 | STEAP2/NM001040665.1 |
| CHAT/NM020549.4 | MCCC1/NM020166.4 | STEAP2/NM152999.3 |
| CHCHD1/NM203298.2 | MCCC2/NM022132.4 | STEAP3/NM001008410.1 |
| CHCHD10/NM213720.2 | MCCD1/NM001011700.2 | STEAP4/NM001205315.1 |
| CHCHD2/NM016139.3 | MCEE/NM032601.3 | STH/NM001007532.2 |
| CHCHD3/NM017812.3 | MCEMP1/NM174918.2 | STIL/NM001282936.1 |
| CHCHD4/NM144636.2 | MCF2L/NM024979.4 | STIM1/NM003156.3 |
| CHCHD5/NM032309.3 | MCF2L2/NM015078.3 | STIM2/NM020860.3 |
| CHCHD6/NM032343.2 | MCFD2/NM001171508.2 | STIP1/NM006819.2 |
| CHCHD7/NM001011671.2 | MCHR1/NM005297.3 | STK10/NM005990.3 |
| CHD1/NM001270.2 | MCHR2/NM001040179.1 | STK11/NM000455.4 |
| CHD1L/NM004284.4 | MCHR2/NM032503.2 | STK11IP/NM052902.3 |
| CHD2/NM001271.3 | MCL1/NM021960.4 | STK16/NM001008910.2 |
| CHD3/NM001005271.2 | MCM10/NM182751.2 | STK17A/NM004760.2 |
| CHD5/NM015557.2 | MCM2/NM004526.3 | STK17B/NM004226.3 |
| CHD6/NM032221.4 | MCM3/NM002388.4 | STK19/NM032454.1 |
| CHD7/NM017780.3 | MCM3AP/NM003906.4 | STK24/NM001032296.3 |
| CHD8/NM020920.3 | MCM4/NM182746.2 | STK25/NM001271977.1 |
| CHD9/NM025134.4 | MCM6/NM005915.5 | STK25/NM001271978.1 |
| CHDH/NM018397.4 | MCM7/NM005916.4 | STK3/NM006281.3 |
| CHEK1/NM001114122.2 | MCM8/NM032485.5 | STK31/NM031414.4 |
| CHEK1/NM001274.5 | MCM9/NM017696.2 | STK32A/NM001112724.1 |
| CHEK2/NM007194.3 | MCMBP/NM024834.3 | STK32B/NM018401.2 |
| CHERP/NM006387.5 | MCMDC2/NM173518.4 | STK32C/NM173575.3 |
| CHFR/NM001161344.1 | MCOLN1/NM020533.2 | STK33/NM030906.3 |
| CHGA/NM001275.3 | MCOLN3/NM018298.10 | STK35/NM080836.3 |
| CHGB/NM001819.2 | MCPH1/NM024596.4 | STK36/NM015690.4 |
| CHI3L1/NM001276.2 | MCRIP1/NM207368.4 | STK38/NM007271.3 |
| CHI3L2/NM004000.2 | MCRIP2/NM138418.2 | STK38L/NM015000.3 |
| CHIA/NM201653.3 | MCRS1/NM006337.4 | STK39/NM013233.2 |
| CHIC2/NM012110.3 | MCTP1/NM001002796.3 | STK4/NM006282.3 |
| CHID1/NM001142675.1 | MCTP2/NM018349.3 | STK40/NM032017.2 |
| CHIT1/NM003465.2 | MCU/NM138357.2 | STKLD1/NM153710.4 |
| CHKA/NM001277.2 | MCUR1/NM001031713.3 | STMN1/NM005563.3 |
| CHL1/NM006614.3 | MDC1/NM014641.2 | STMN1/NM203399.1 |
| CHML/NM001821.3 | MDFI/NM001300804.1 | STMN1/NM203401.1 |
| CHMP1A/NM002768.4 | MDFI/NM005586.3 | STMN2/NM007029.3 |
| CHMP1B/NM020412.4 | MDGA1/NM153487.3 | STMN3/NM015894.3 |
| CHMP2A/NM014453.3 | MDGA2/NM001113498.2 | STMN4/NM030795.3 |
| CHMP2A/NM198426.2 | MDH1/NM005917.3 | STMND1/NM001190766.1 |
| CHMP2B/NM014043.3 | MDH1B/NM001039845.2 | STOM/NM004099.5 |
| CHMP3/NM016079.3 | MDH2/NM005918.3 | STOML1/NM004809.4 |
| CHMP4A/NM014169.3 | MDK/NM001012333.2 | STOML2/NM013442.2 |
| CHMP4B/NM176812.4 | MDK/NM001012334.2 | STOML3/NM145286.2 |
| CHMP4C/NM152284.3 | MDK/NM001270550.1 | STON1-GTF2A1L/NM172311.2 |
| CHMP5/NM016410.5 | MDK/NM002391.4 | STON1/NM006873.3 |
| CHMP7/NM152272.4 | MDM1/NM017440.4 | STOX1/NM152709.4 |
| CHN1/NM001822.5 | MDM4/NM002393.4 | STOX2/NM020225.2 |
| CHN2/NM004067.3 | MDN1/NM014611.2 | STPG1/NM178122.4 |
| CHODL/NM024944.2 | MDP1/NM138476.3 | STPG2/NM174952.2 |
| CHORDC1/NM012124.2 | ME1/NM002395.5 | STPG3/NM001004353.3 |
| CHP2/NM022097.3 | ME2/NM002396.4 | STRA13/NM001271006.1 |
| CHPF/NM024536.5 | ME3/NM001014811.1 | STRA6/NM001142617.1 |
| CHPF2/NM019015.2 | ME3/NM001161586.1 | STRA6/NM001142618.1 |
| CHPT1/NM020244.2 | ME3/NM006680.2 | STRA6/NM022369.3 |
| CHRAC1/NM017444.5 | MEA1/NM001318942.1 | STRA8/NM182489.1 |
| CHRD/NM003741.3 | MEA1/NM001318943.1 | STRADA/NM001003787.2 |
| CHRDL2/NM001278473.2 | MEAF6/NM022756.5 | STRADB/NM018571.5 |
| CHRFAM7A/NM139320.1 | MECOM/NM004991.3 | STRAP/NM007178.3 |
| CHRM1/NM000738.2 | MECR/NM001024732.2 | STRBP/NM018387.4 |
| CHRM2/NM001006626.1 | MED1/NM004774.3 | STRC/NM153700.2 |
| CHRM2/NM001006627.1 | MED10/NM032286.2 | STRIP2/NM020704.2 |
| CHRM2/NM001006628.1 | MED11/NM001001683.3 | STRN/NM003162.3 |
| CHRM2/NM001006629.1 | MED12L/NM053002.5 | STRN3/NM014574.3 |
| CHRM2/NM001006631.1 | MED13/NM005121.2 | STRN4/NM013403.2 |
| CHRM3/NM000740.2 | MED13L/NM015335.4 | STT3A/NM152713.4 |
| CHRM4/NM000741.3 | MED15/NM015889.4 | STT3B/NM178862.2 |
| CHRM5/NM001320917.1 | MED16/NM005481.2 | STUB1/NM005861.3 |
| CHRM5/NM012125.3 | MED17/NM004268.4 | STUM/NM001003665.3 |
| CHRNA1/NM000079.3 | MED18/NM001127350.1 | STX10/NM003765.2 |
| CHRNA10/NM020402.3 | MED19/NM001317078.1 | STX11/NM003764.3 |
| CHRNA3/NM000743.4 | MED20/NM004275.4 | STX12/NM177424.2 |
| CHRNA4/NM000744.6 | MED21/NM004264.4 | STX16/NM001001433.2 |
| CHRNA5/NM000745.3 | MED22/NM133640.4 | STX17/NM017919.2 |
| CHRNA6/NM004198.3 | MED23/NM004830.3 | STX18/NM016930.2 |
| CHRNA7/NM000746.5 | MED24/NM014815.3 | STX19/NM001001850.2 |
| CHRNA9/NM017581.3 | MED26/NM004831.3 | STX1A/NM004603.3 |
| CHRNB1/NM000747.2 | MED27/NM004269.3 | STX1B/NM052874.4 |
| CHRNB2/NM000748.2 | MED28/NM025205.4 | STX3/NM004177.4 |
| CHRND/NM000751.2 | MED29/NM017592.2 | STX4/NM004604.4 |
| CHRNE/NM000080.3 | MED30/NM080651.3 | STX5/NM003164.4 |
| CHRNG/NM005199.4 | MED31/NM016060.2 | STX6/NM005819.5 |
| CHST1/NM003654.5 | MED4/NM014166.3 | STX8/NM004853.2 |
| CHST10/NM004854.4 | MED6/NM005466.3 | STXBP1/NM003165.3 |
| CHST11/NM018413.5 | MED7/NM001100816.1 | STXBP2/NM006949.3 |
| CHST13/NM152889.2 | MED7/NM004270.4 | STXBP3/NM007269.2 |
| CHST14/NM130468.3 | MED8/NM201542.4 | STXBP4/NM178509.5 |
| CHST15/NM001270764.1 | MEDAG/NM032849.3 | STXBP5/NM139244.4 |
| CHST15/NM015892.4 | MEF2A/NM005587.3 | STXBP6/NM001304476.1 |
| CHST2/NM004267.4 | MEF2B/NM001145785.1 | STXBP6/NM001304477.1 |
| CHST3/NM004273.4 | MEF2C/NM001131005.2 | STXBP6/NM014178.7 |
| CHST4/NM001166395.1 | MEFV/NM000243.2 | STYK1/NM018423.2 |
| CHST4/NM005769.2 | MEGF11/NM032445.2 | STYX/NM001130701.1 |
| CHST5/NM024533.4 | MEGF6/NM001409.3 | STYXL1/NM016086.2 |
| CHST6/NM021615.4 | MEGF8/NM001410.2 | SUCLA2/NM003850.2 |
| CHST8/NM001127895.1 | MEGF9/NM001080497.2 | SUCLG1/NM003849.3 |
| CHST8/NM001127896.1 | MEI1/NM152513.3 | SUCLG2/NM003848.3 |
| CHST8/NM022467.3 | MEI4/NM001282136.2 | SUCNR1/NM033050.5 |
| CHST9/NM031422.5 | MEIG1/NM001080836.2 | SUCO/NM014283.4 |
| CHSY1/NM014918.4 | MEIKIN/NM001303622.1 | SUDS3/NM022491.2 |
| CHSY3/NM175856.4 | MEIOB/NM152764.2 | SUFU/NM016169.3 |
| CHTF18/NM022092.2 | MEIOC/NM001145080.2 | SUGCT/NM001193311.1 |
| CHTF8/NM001039690.3 | MEIS1/NM002398.2 | SUGP1/NM172231.3 |
| CHTF8/NM001040146.3 | MEIS2/NM170675.4 | SUGP2/NM001017392.4 |
| CHTOP/NM015607.3 | MEIS3/NM020160.2 | SUGT1/NM006704.4 |
| CHUK/NM001278.4 | MELK/NM014791.3 | SULF1/NM001128204.1 |
| CHURC1-FNTB/NM001202559.1 | MELTF/NM005929.5 | SULF1/NM001128206.1 |
| CIAO1/NM004804.2 | MEMO1/NM001301833.1 | SULF2/NM001161841.1 |
| CIAPIN1/NM020313.3 | MEMO1/NM015955.3 | SULF2/NM018837.3 |
| CIART/NM001300838.1 | MEN1/NM130799.2 | SULT1A1/NM177529.2 |
| CIB1/NM006384.3 | MEOX1/NM004527.3 | SULT1A1/NM177534.2 |
| CIB2/NM006383.3 | MEOX2/NM005924.4 | SULT1A2/NM001054.3 |
| CIB3/NM054113.3 | MEP1A/NM005588.2 | SULT1A2/NM177528.2 |
| CIB4/NM001029881.2 | MEP1B/NM005925.2 | SULT1A3/NM177552.3 |
| CIC/NM001304815.1 | MEPCE/NM019606.5 | SULT1A4/NM001017390.2 |
| CIDEA/NM001279.3 | MEPE/NM001184694.2 | SULT1B1/NM014465.3 |
| CIDEC/NM001199552.1 | MEPE/NM020203.3 | SULT1C3/NM001008743.2 |
| CIDEC/NM022094.3 | MERTK/NM006343.2 | SULT1C4/NM006588.3 |
| CIITA/NM000246.3 | MESDC1/NM022566.2 | SULT1E1/NM005420.2 |
| CILP/NM003613.3 | MESDC2/NM015154.2 | SULT2A1/NM003167.3 |
| CILP2/NM153221.2 | MESP1/NM018670.3 | SULT2B1/NM177973.1 |
| CINP/NM032630.2 | MESP2/NM001039958.1 | SULT4A1/NM014351.3 |
| CIPC/NM033426.2 | MEST/NM002402.3 | SULT6B1/NM001032377.1 |
| CIR1/NM004882.3 | MET/NM000245.3 | SUMF1/NM182760.3 |
| CIRBP/NM001280.2 | METAP1/NM015143.2 | SUMF2/NM015411.2 |
| CISD1/NM018464.4 | METAP1D/NM199227.2 | SUMO2/NM006937.3 |
| CISD2/NM001008388.4 | METAP2/NM006838.3 | SUMO4/NM001002255.1 |
| CISD3/NM001136498.1 | METRN/NM024042.3 | SUN1/NM025154.5 |
| CISH/NM145071.2 | METRNL/NM001004431.2 | SUN2/NM001199580.1 |
| CIT/NM001206999.1 | METTL1/NM005371.5 | SUN2/NM015374.2 |
| CITED4/NM133467.2 | METTL10/NM212554.3 | SUN3/NM001030019.1 |
| CIZ1/NM001131016.1 | METTL11B/NM001136107.1 | SUN5/NM080675.3 |
| CIZ1/NM012127.2 | METTL12/NM001043229.1 | SUOX/NM001032387.1 |
| CKAP2/NM018204.4 | METTL13/NM015935.4 | SUPT16H/NM007192.3 |
| CKAP2L/NM152515.4 | METTL14/NM020961.3 | SUPT20H/NM001278482.1 |
| CKAP4/NM006825.3 | METTL15/NM001297775.1 | SUPT20H/NM017569.3 |
| CKAP5/NM001008938.3 | METTL16/NM024086.3 | SUPT3H/NM181356.2 |
| CKB/NM001823.4 | METTL17/NM022734.2 | SUPT5H/NM001130824.1 |
| CKM/NM001824.4 | METTL21A/NM001127395.2 | SUPT5H/NM003169.3 |
| CKMT1A/NM001015001.2 | METTL21A/NM145280.5 | SUPT6H/NM003170.4 |
| CKMT1A/NM001321926.1 | METTL21B/NM015433.2 | SUPT7L/NM001282729.1 |
| CKMT1B/NM020990.4 | METTL21C/NM001010977.2 | SUPV3L1/NM003171.4 |
| CKMT2/NM001099735.1 | METTL22/NM024109.3 | SURF1/NM003172.3 |
| CKS1B/NM001826.2 | METTL23/NM001206984.2 | SURF2/NM017503.4 |
| CKS2/NM001827.2 | METTL24/NM001123364.1 | SURF4/NM033161.3 |
| CLASP1/NM015282.2 | METTL25/NM032230.2 | SURF6/NM006753.5 |
| CLASP2/NM015097.2 | METTL2A/NM181725.3 | SUSD1/NM022486.4 |
| CLASRP/NM007056.2 | METTL2B/NM018396.2 | SUSD2/NM019601.3 |
| CLC/NM001828.5 | METTL3/NM019852.4 | SUSD3/NM145006.3 |
| CLCA1/NM001285.3 | METTL4/NM022840.4 | SUSD4/NM017982.3 |
| CLCA4/NM012128.3 | METTL6/NM152396.3 | SUSD5/NM015551.1 |
| CLCC1/NM015127.4 | METTL7A/NM014033.3 | SUSD6/NM014734.3 |
| CLCF1/NM013246.2 | METTL7B/NM152637.2 | SUV39H2/NM001193425.1 |
| CLCN1/NM000083.2 | METTL8/NM024770.4 | SUV39H2/NM024670.3 |
| CLCN2/NM004366.5 | METTL9/NM016025.4 | SUZ12/NM015355.3 |
| CLCN3/NM173872.3 | MEX3A/NM001093725.1 | SV2A/NM014849.4 |
| CLCN6/NM001286.3 | MEX3B/NM032246.4 | SV2B/NM001323031.1 |
| CLCN7/NM001287.5 | MEX3C/NM016626.4 | SV2B/NM001323032.1 |
| CLCNKA/NM004070.3 | MEX3D/NM203304.3 | SV2B/NM001323038.1 |
| CLCNKB/NM000085.4 | MFAP1/NM005926.2 | SV2B/NM001323039.1 |
| CLDN1/NM021101.4 | MFAP2/NM002403.3 | SV2C/NM014979.3 |
| CLDN10/NM006984.4 | MFAP2/NM017459.2 | SVBP/NM199342.3 |
| CLDN12/NM012129.4 | MFAP3L/NM021647.7 | SVEP1/NM153366.3 |
| CLDN14/NM001146077.1 | MFAP4/NM002404.2 | SVIL/NM021738.2 |
| CLDN14/NM001146078.2 | MFAP5/NM003480.3 | SVOP/NM018711.4 |
| CLDN14/NM012130.3 | MFF/NM001277061.1 | SVOPL/NM001139456.1 |
| CLDN14/NM144492.2 | MFF/NM020194.5 | SWAP70/NM015055.3 |
| CLDN15/NM001185080.1 | MFGE8/NM005928.3 | SWI5/NM001040011.1 |
| CLDN15/NM014343.2 | MFHAS1/NM004225.2 | SWSAP1/NM175871.3 |
| CLDN16/NM006580.3 | MFN1/NM033540.2 | SWT1/NM001105518.1 |
| CLDN17/NM012131.2 | MFN2/NM001127660.1 | SWT1/NM017673.6 |
| CLDN18/NM016369.3 | MFNG/NM002405.3 | SYBU/NM001099743.1 |
| CLDN19/NM148960.2 | MFRP/NM031433.3 | SYBU/NM001099751.1 |
| CLDN20/NM001001346.3 | MFSD1/NM022736.2 | SYBU/NM001099753.1 |
| CLDN22/NM001111319.1 | MFSD10/NM001120.4 | SYBU/NM017786.5 |
| CLDN23/NM194284.2 | MFSD10/NM001146069.1 | SYCE1/NM130784.2 |
| CLDN24/NM001185149.1 | MFSD11/NM001242534.1 | SYCE1L/NM001129979.1 |
| CLDN25/NM001101389.1 | MFSD11/NM024311.3 | SYCE2/NM001105578.1 |
| CLDN3/NM001306.3 | MFSD12/NM174983.4 | SYCE3/NM001123225.1 |
| CLDN4/NM001305.4 | MFSD13A/NM024789.3 | SYCN/NM001080468.2 |
| CLDN5/NM001130861.1 | MFSD14A/NM033055.2 | SYCP1/NM003176.3 |
| CLDN7/NM001185022.1 | MFSD14B/NM032558.2 | SYCP2/NM014258.3 |
| CLDN7/NM001307.5 | MFSD2A/NM032793.4 | SYCP2L/NM001040274.2 |
| CLDN8/NM199328.2 | MFSD2B/NM001080473.1 | SYCP3/NM001177949.1 |
| CLDN9/NM020982.3 | MFSD3/NM138431.2 | SYDE1/NM033025.5 |
| CLDND1/NM001040199.1 | MFSD4A/NM181644.4 | SYDE2/NM032184.1 |
| CLDND2/NM152353.2 | MFSD4B/NM153369.2 | SYF2/NM015484.4 |
| CLEC10A/NM182906.2 | MFSD5/NM032889.4 | SYK/NM001174167.2 |
| CLEC11A/NM002975.2 | MFSD6/NM017694.3 | SYK/NM003177.6 |
| CLEC12A/NM138337.5 | MFSD6L/NM152599.3 | SYMPK/NM004819.2 |
| CLEC12B/NM001129998.2 | MFSD7/NM032219.3 | SYN2/NM133625.4 |
| CLEC14A/NM175060.2 | MFSD8/NM152778.2 | SYN3/NM003490.3 |
| CLEC16A/NM015226.2 | MFSD9/NM032718.4 | SYNC/NM030786.2 |
| CLEC17A/NM001204118.1 | MGA/NM001164273.1 | SYNCRIP/NM006372.4 |
| CLEC18A/NM001136214.2 | MGAM2/NM001293626.1 | SYNDIG1/NM001323607.1 |
| CLEC18A/NM001271197.1 | MGARP/NM032623.3 | SYNDIG1/NM024893.2 |
| CLEC18A/NM182619.3 | MGAT1/NM001114617.1 | SYNDIG1L/NM001105579.1 |
| CLEC18B/NM001011880.2 | MGAT1/NM001114618.1 | SYNE1/NM182961.3 |
| CLEC18C/NM173619.3 | MGAT1/NM001114620.1 | SYNE2/NM182914.2 |
| CLEC19A/NM001256720.1 | MGAT1/NM002406.3 | SYNE3/NM152592.3 |
| CLEC1A/NM016511.3 | MGAT2/NM002408.3 | SYNE4/NM001039876.2 |
| CLEC1B/NM016509.3 | MGAT3/NM001098270.1 | SYNGAP1/NM006772.2 |
| CLEC2B/NM005127.2 | MGAT3/NM002409.4 | SYNGR1/NM004711.4 |
| CLEC2D/NM013269.5 | MGAT4A/NM012214.2 | SYNGR2/NM004710.4 |
| CLEC2L/NM001080511.2 | MGAT4B/NM014275.4 | SYNGR3/NM004209.5 |
| CLEC3A/NM005752.4 | MGAT4C/NM013244.3 | SYNGR4/NM012451.3 |
| CLEC3B/NM003278.2 | MGAT5/NM002410.4 | SYNJ2/NM003898.3 |
| CLEC4A/NM016184.3 | MGAT5B/NM144677.2 | SYNJ2BP-COX16/NM001202548.2 |
| CLEC4C/NM130441.2 | MGC57346-CRHR1/NM001303016.1 | SYNM/NM145728.2 |
| CLEC4D/NM080387.4 | MGLL/NM001003794.2 | SYNPO/NM007286.5 |
| CLEC4E/NM014358.3 | MGME1/NM052865.3 | SYNPO2/NM133477.2 |
| CLEC4F/NM173535.2 | MGMT/NM002412.4 | SYNPO2L/NM001114133.2 |
| CLEC4G/NM198492.3 | MGP/NM000900.3 | SYNPR/NM001130003.1 |
| CLEC5A/NM013252.2 | MGRN1/NM015246.3 | SYNRG/NM007247.5 |
| CLEC6A/NM001007033.1 | MGST1/NM001260511.1 | SYPL1/NM006754.3 |
| CLEC7A/NM022570.4 | MGST1/NM145764.2 | SYPL2/NM001040709.1 |
| CLEC9A/NM207345.3 | MGST1/NM145791.2 | SYS1/NM001197129.1 |
| CLECL1/NM172004.3 | MGST1/NM145792.2 | SYS1/NM033542.3 |
| CLGN/NM001130675.1 | MGST2/NM001204366.1 | SYT1/NM001135805.1 |
| CLHC1/NM152385.2 | MGST3/NM004528.3 | SYT1/NM001135806.1 |
| CLIC1/NM001287593.1 | MIA/NM001202553.1 | SYT1/NM005639.2 |
| CLIC1/NM001287594.1 | MIA/NM006533.3 | SYT10/NM198992.3 |
| CLIC1/NM001288.4 | MIA3/NM198551.3 | SYT11/NM152280.4 |
| CLIC3/NM004669.2 | MIB1/NM020774.3 | SYT12/NM001177880.1 |
| CLIC4/NM013943.2 | MIB2/NM080875.2 | SYT12/NM177963.3 |
| CLIC5/NM016929.4 | MICA/NM001177519.2 | SYT13/NM020826.2 |
| CLIC6/NM053277.2 | MICAL1/NM022765.3 | SYT14/NM153262.3 |
| CLINT1/NM014666.3 | MICAL2/NM001282663.1 | SYT15/NM031912.4 |
| CLIP1/NM002956.2 | MICAL3/NM015241.2 | SYT16/NM031914.2 |
| CLIP2/NM003388.4 | MICALCL/NM032867.2 | SYT17/NM016524.3 |
| CLIP3/NM001199570.1 | MICALL1/NM033386.3 | SYT2/NM001136504.1 |
| CLIP3/NM015526.2 | MICALL2/NM182924.3 | SYT2/NM177402.4 |
| CLIP4/NM001287527.1 | MICB/NM005931.4 | SYT3/NM001160328.1 |
| CLK2/NM003993.3 | MICU1/NM001195518.1 | SYT3/NM032298.2 |
| CLK3/NM003992.4 | MICU2/NM152726.2 | SYT4/NM020783.3 |
| CLK4/NM020666.2 | MICU3/NM181723.2 | SYT5/NM003180.2 |
| CLLU1/NM001025233.2 | MIDN/NM177401.4 | SYT6/NM001270805.1 |
| CLLU1OS/NM001025232.1 | MIEF1/NM019008.5 | SYT6/NM205848.3 |
| CLMN/NM024734.3 | MIEF2/NM139162.3 | SYT7/NM004200.3 |
| CLMP/NM024769.3 | MIEN1/NM032339.4 | SYT8/NM138567.4 |
| CLN3/NM000086.2 | MIER1/NM001077701.2 | SYT9/NM175733.3 |
| CLN3/NM001042432.1 | MIER2/NM017550.1 | SYTL1/NM032872.2 |
| CLN5/NM006493.2 | MIER3/NM152622.4 | SYTL2/NM032943.4 |
| CLN6/NM017882.2 | MIF/NM002415.1 | SYTL3/NM001242384.1 |
| CLN8/NM018941.3 | MIF4GD/NM001242501.1 | SYVN1/NM032431.2 |
| CLNK/NM052964.2 | MIIP/NM021933.3 | SZRD1/NM001114600.2 |
| CLNS1A/NM001293.2 | MILR1/NM001085423.1 | SZT2/NM015284.3 |
| CLOCK/NM001267843.1 | MINA/NM032778.5 | TAAR1/NM138327.2 |
| CLOCK/NM004898.3 | MINK1/NM153827.4 | TAAR2/NM014626.3 |
| CLP1/NM006831.2 | MINOS1/NM001032363.3 | TAAR5/NM003967.2 |
| CLPB/NM030813.5 | MINPP1/NM004897.4 | TAAR6/NM175067.1 |
| CLPP/NM006012.2 | MIOS/NM019005.3 | TAAR8/NM053278.1 |
| CLPSL1/NM001010886.3 | MIOX/NM017584.5 | TAAR9/NM175057.3 |
| CLPSL2/NM207409.3 | MIP/NM012064.3 | TAB1/NM006116.2 |
| CLPTM1/NM001294.3 | MIPEP/NM005932.3 | TAB2/NM001292034.2 |
| CLPTM1L/NM030782.4 | MIPOL1/NM138731.6 | TAB2/NM015093.5 |
| CLPX/NM006660.4 | MIR1-1HG/NM001302812.1 | TAC1/NM003182.2 |
| CLRN1/NM174878.2 | MIR205HG/NM001104548.1 | TAC3/NM013251.3 |
| CLRN2/NM001079827.2 | MIS12/NM001258220.1 | TAC4/NM170685.2 |
| CLRN3/NM152311.3 | MIS12/NM024039.2 | TACC1/NM001122824.1 |
| CLSPN/NM022111.3 | MIS18A/NM018944.2 | TACC2/NM206862.3 |
| CLSTN1/NM014944.4 | MIS18BP1/NM018353.4 | TACC3/NM006342.2 |
| CLSTN2/NM022131.2 | MISP/NM173481.3 | TACO1/NM016360.3 |
| CLTA/NM007096.3 | MITD1/NM138798.2 | TACR1/NM001058.3 |
| CLTB/NM007097.3 | MITF/NM198159.2 | TACR2/NM001057.2 |
| CLTC/NM004859.3 | MIXL1/NM031944.2 | TACR3/NM001059.2 |
| CLTCL1/NM007098.3 | MKI67/NM002417.4 | TACSTD2/NM002353.2 |
| CLU/NM001831.3 | MKKS/NM018848.3 | TADA1/NM053053.3 |
| CLUAPI/NM015041.2 | MKKS/NM170784.2 | TADA2A/NM001166105.2 |
| CLUH/NM015229.3 | MKL1/NM001318139.1 | TADA2A/NM001488.4 |
| CLUL1/NM001318522.1 | MKL2/NM014048.4 | TADA2B/NM152293.2 |
| CLUL1/NM014410.4 | MKLN1/NM013255.4 | TADA3/NM006354.3 |
| CLUL1/NM199167.1 | MKNK2/NM199054.2 | TAF10/NM006284.3 |
| CLVS1/NM173519.2 | MKRN1/NM013446.3 | TAF11/NM005643.3 |
| CLYBL/NM206808.3 | MKRN2/NM014160.4 | TAF12/NM001135218.1 |
| CMA1/NM001836.4 | MKRN2OS/NM001195279.1 | TAF13/NM005645.3 |
| CMAS/NM018686.5 | MKRN3/NM005664.3 | TAF15/NM139215.2 |
| CMBL/NM138809.3 | MKS1/NM017777.3 | TAF1A/NM001201536.1 |
| CMC2/NM020188.3 | MKX/NM173576.2 | TAF1B/NM005680.2 |
| CMIP/NM198390.2 | MLANA/NM005511.1 | TAF1C/NM005679.3 |
| CMKLR1/NM004072.2 | MLC1/NM015166.3 | TAF1D/NM024116.3 |
| CMPK1/NM016308.2 | MLC1/NM139202.2 | TAF1L/NM153809.2 |
| CMPK2/NM207315.3 | MLEC/NM014730.3 | TAF2/NM003184.3 |
| CMSS1/NM032359.3 | MLF1/NM022443.4 | TAF3/NM031923.3 |
| CMTM1/NM052999.3 | MLF2/NM005439.2 | TAF4/NM003185.3 |
| CMTM2/NM144673.2 | MLH1/NM000249.3 | TAF4B/NM005640.2 |
| CMTM3/NM144601.3 | MLH3/NM001040108.1 | TAF5/NM006951.4 |
| CMTM3/NM181553.2 | MLKL/NM152649.3 | TAF5L/NM014409.3 |
| CMTM4/NM181521.2 | MLLT1/NM005934.3 | TAF6L/NM006473.3 |
| CMTM5/NM138460.2 | MLLT10/NM004641.3 | TAF7/NM005642.2 |
| CMTM6/NM017801.2 | MLLT11/NM006818.3 | TAF8/NM138572.2 |
| CMTM7/NM138410.3 | MLLT3/NM004529.3 | TAF9/NM001015892.1 |
| CMTM8/NM178868.4 | MLLT4/NM001291964.1 | TAGAP/NM054114.4 |
| CMTR1/NM015050.2 | MLLT6/NM005937.3 | TAGLN/NM003186.3 |
| CMYA5/NM153610.4 | MLN/NM002418.2 | TAGLN2/NM001277223.1 |
| CNBD1/NM173538.2 | MLNR/NM001507.1 | TAGLN2/NM003564.2 |
| CNBD2/NM080834.3 | MLPH/NM024101.6 | TAGLN3/NM001008272.1 |
| CNBP/NM003418.4 | MLST8/NM001199173.1 | TAGLN3/NM001008273.1 |
| CNDP1/NM032649.5 | MLST8/NM001199174.1 | TAL1/NM001287347.2 |
| CNDP2/NM018235.2 | MLX/NM170607.2 | TAL1/NM001290404.1 |
| CNEP1R1/NM001281789.1 | MLXIP/NM014938.5 | TAL1/NM003189.5 |
| CNFN/NM032488.3 | MLXIPL/NM032951.2 | TAL2/NM005421.2 |
| CNGA1/NM000087.3 | MLYCD/NM012213.2 | TALDO1/NM006755.1 |
| CNGA4/NM001037329.3 | MMAA/NM172250.2 | TAMM41/NM001284401.1 |
| CNGB1/NM001297.4 | MMACHC/NM015506.2 | TANC1/NM033394.2 |
| CNGB3/NM019098.4 | MMADHC/NM015702.2 | TANC2/NM025185.3 |
| CNIH1/NM005776.2 | MMD/NM012329.2 | TANGO2/NM001283106.2 |
| CNIH2/NM182553.2 | MMD2/NM001100600.1 | TANGO2/NM001283116.2 |
| CNIH3/NM152495.1 | MME/NM000902.3 | TANGO6/NM024562.1 |
| CNIH4/NM014184.3 | MME/NM007287.2 | TANK/NM001199135.1 |
| CNKSR1/NM006314.2 | MME/NM007288.2 | TANK/NM004180.2 |
| CNKSR3/NM173515.2 | MME/NM007289.2 | TAOK1/NM020791.2 |
| CNN1/NM001299.5 | MMEL1/NM033467.3 | TAOK2/NM004783.3 |
| CNN2/NM004368.3 | MMP1/NM002421.3 | TAOK3/NM016281.3 |
| CNN3/NM001839.4 | MMP10/NM002425.2 | TAP1/NM000593.5 |
| CNNM1/NM020348.2 | MMP11/NM005940.4 | TAP2/NM018833.2 |
| CNNM2/NM017649.4 | MMP12/NM002426.5 | TAPBP/NM172208.2 |
| CNNM3/NM017623.4 | MMP13/NM002427.3 | TAPBPL/NM018009.4 |
| CNNM4/NM020184.3 | MMP14/NM004995.3 | TAPT1/NM153365.2 |
| CNOT10/NM015442.2 | MMP15/NM002428.3 | TARBP1/NM005646.3 |
| CNOT11/NM017546.4 | MMP16/NM005941.4 | TARM1/NM001135686.2 |
| CNOT2/NM001199302.1 | MMP17/NM016155.5 | TARS/NM001258437.1 |
| CNOT2/NM001199303.1 | MMP19/NM002429.5 | TARS/NM152295.4 |
| CNOT2/NM014515.5 | MMP2/NM004530.5 | TARS2/NM025150.4 |
| CNOT3/NM014516.3 | MMP20/NM004771.3 | TARSL2/NM152334.2 |
| CNOT4/NM013316.3 | MMP21/NM147191.1 | TAS1R1/NM138697.3 |
| CNOT6/NM001303241.1 | MMP23B/NM006983.1 | TAS1R2/NM152232.2 |
| CNOT7/NM001322090.1 | MMP24/NM006690.3 | TAS1R3/NM152228.2 |
| CNOT7/NM001322092.1 | MMP25/NM022468.4 | TAS2R1/NM019599.2 |
| CNOT8/NM001301073.1 | MMP26/NM021801.3 | TAS2R10/NM023921.1 |
| CNOT9/NM005444.2 | MMP27/NM022122.2 | TAS2R13/NM023920.2 |
| CNP/NM033133.4 | MMP28/NM024302.4 | TAS2R14/NM023922.1 |
| CNPPD1/NM001321389.1 | MMP3/NM002422.4 | TAS2R16/NM016945.2 |
| CNPPD1/NM001321390.1 | MMP7/NM002423.4 | TAS2R19/NM176888.2 |
| CNPPD1/NM001321391.1 | MMP8/NM002424.2 | TAS2R20/NM176889.2 |
| CNPPD1/NM015680.5 | MMP9/NM004994.2 | TAS2R3/NM016943.2 |
| CNPY2/NM014255.5 | MMRN1/NM007351.2 | TAS2R30/NM001097643.1 |
| CNPY3-GNMT/NM001318856.1 | MMRN2/NM024756.2 | TAS2R31/NM176885.2 |
| CNR1/NM001160259.1 | MMS19/NM001289405.1 | TAS2R38/NM176817.4 |
| CNR2/NM001841.2 | MMS19/NM022362.4 | TAS2R39/NM176881.2 |
| CNRIP1/NM015463.2 | MMS22L/NM198468.2 | TAS2R4/NM016944.1 |
| CNST/NM152609.2 | MN1/NM002430.2 | TAS2R40/NM176882.1 |
| CNTD1/NM173478.2 | MNAT1/NM002431.3 | TAS2R41/NM176883.2 |
| CNTF/NM000614.3 | MND1/NM032117.3 | TAS2R42/NM181429.2 |
| CNTFR/NM001207011.1 | MNDA/NM002432.1 | TAS2R43/NM176884.2 |
| CNTFR/NM147164.2 | MNS1/NM018365.2 | TAS2R46/NM176887.2 |
| CNTLN/NM017738.3 | MNT/NM020310.2 | TAS2R5/NM018980.2 |
| CNTN1/NM001843.3 | MNX1/NM005515.3 | TAS2R50/NM176890.2 |
| CNTN2/NM005076.3 | MOB1A/NM018221.4 | TAS2R60/NM177437.1 |
| CNTN3/NM020872.2 | MOB1B/NM173468.3 | TAS2R7/NM023919.2 |
| CNTN4/NM001206955.1 | MOB2/NM001172223.2 | TAS2R8/NM023918.1 |
| CNTN4/NM175607.2 | MOB3A/NM130807.2 | TAS2R9/NM023917.2 |
| CNTNAP1/NM003632.2 | MOB3B/NM024761.4 | TASP1/NM017714.2 |
| CNTNAP2/NM014141.5 | MOB3C/NM201403.2 | TAT/NM000353.2 |
| CNTNAP3/NM033655.3 | MOB4/NM015387.4 | TATDN3/NM001042552.2 |
| CNTNAP3B/NM001201380.2 | MOBP/NM001278323.1 | TAX1BP3/NM014604.3 |
| CNTNAP5/NM130773.3 | MOCOS/NM017947.2 | TBATA/NM152710.3 |
| CNTROB/NM053051.3 | MOCS2/NM004531.4 | TBC1D1/NM015173.3 |
| COA1/NM018224.3 | MOCS3/NM014484.4 | TBC1D10B/NM015527.3 |
| COA3/NM001040431.2 | MOG/NM002433.4 | TBC1D10C/NM198517.3 |
| COA4/NM016565.2 | MOGAT1/NM058165.2 | TBC1D13/NM018201.4 |
| COA5/NM001008215.2 | MOGAT2/NM025098.3 | TBC1D14/NM001113361.1 |
| COA6/NM001301733.1 | MOGAT3/NM178176.3 | TBC1D14/NM020773.2 |
| COA7/NM023077.2 | MOGS/NM006302.2 | TBC1D15/NM022771.4 |
| COBL/NM015198.3 | MOK/NM014226.2 | TBC1D16/NM019020.3 |
| COBLL1/NM014900.4 | MON1B/NM014940.3 | TBC1D17/NM024682.2 |
| COCH/NM004086.2 | MON2/NM015026.2 | TBC1D19/NM018317.3 |
| COG1/NM018714.2 | MORC2/NM001303256.2 | TBC1D2/NM001267571.1 |
| COG2/NM007357.2 | MORC3/NM015358.2 | TBC1D20/NM144628.3 |
| COG3/NM031431.3 | MORF4L1/NM206839.2 | TBC1D21/NM153356.2 |
| COG4/NM015386.2 | MORN1/NM024848.2 | TBC1D22A/NM014346.3 |
| COG6/NM020751.2 | MORN2/NM001145450.1 | TBC1D22B/NM017772.3 |
| COG7/NM153603.3 | MORN3/NM173855.4 | TBC1D23/NM018309.4 |
| COG8/NM032382.4 | MORN4/NM001098831.1 | TBC1D24/NM020705.2 |
| COIL/NM004645.2 | MORN4/NM178832.3 | TBC1D26/NM178571.4 |
| COL10A1/NM000493.3 | MORN5/NM198469.3 | TBC1D28/NM001039397.2 |
| COL11A1/NM001854.3 | MOS/NM005372.1 | TBC1D29/NM015594.2 |
| COL11A2/NM080680.2 | MOSPD3/NM001040097.1 | TBC1D2B/NM015079.5 |
| COL12A1/NM004370.5 | MOSPD3/NM001040098.1 | TBC1D3/NM001123391.3 |
| COL14A1/NM021110.3 | MOSPD3/NM023948.4 | TBC1D30/NM015279.1 |
| COL15A1/NM001855.4 | MOV10/NM001130079.2 | TBC1D31/NM145647.3 |
| COL16A1/NM001856.3 | MOV10/NM001321324.1 | TBC1D3B/NM001001417.6 |
| COL17A1/NM000494.3 | MOV10/NM020963.4 | TBC1D3C/NM001001418.6 |
| COL18A1/NM030582.3 | MOV10L1/NM018995.2 | TBC1D3E/NM001291466.1 |
| COL19A1/NM001858.5 | MOXD1/NM015529.3 | TBC1D3F/NM032258.4 |
| COL1A1/NM000088.3 | MPC1/NM016098.3 | TBC1D3G/NM001291462.1 |
| COL1A2/NM000089.3 | MPC2/NM001143674.3 | TBC1D3H/NM001123392.3 |
| COL20A1/NM020882.2 | MPDU1/NM004870.3 | TBC1D3I/NM001291463.1 |
| COL21A1/NM030820.3 | MPDZ/NM003829.4 | TBC1D3K/NM001291464.1 |
| COL22A1/NM152888.2 | MPEG1/NM001039396.1 | TBC1D3L/NM001291459.1 |
| COL23A1/NM173465.3 | MPG/NM002434.3 | TBC1D4/NM014832.4 |
| COL24A1/NM152890.5 | MPHOSPH10/NM005791.2 | TBC1D7-LOC100130357/NM001318809.1 |
| COL26A1/NM001278563.2 | MPHOSPH6/NM005792.2 | TBC1D8/NM001102426.1 |
| COL27A1/NM032888.3 | MPHOSPH8/NM017520.3 | TBC1D9/NM015130.2 |
| COL28A1/NM001037763.2 | MPHOSPH9/NM022782.3 | TBC1D9B/NM015043.3 |
| COL2A1/NM001844.4 | MPI/NM002435.2 | TBCA/NM004607.2 |
| COL3A1/NM000090.3 | MPL/NM005373.2 | TBCB/NM001281.2 |
| COL4A1/NM001845.5 | MPLKIP/NM138701.3 | TBCC/NM003192.2 |
| COL4A2-AS2/NM001267044.1 | MPND/NM032868.5 | TBCCD1/NM001134415.1 |
| COL4A2/NM001846.2 | MPO/NM000250.1 | TBCCD1/NM018138.4 |
| COL4A3/NM000091.4 | MPP2/NM001278381.1 | TBCD/NM005993.4 |
| COL4A3BP/NM005713.2 | MPP3/NM001932.4 | TBCE/NM001287801.1 |
| COL4A4/NM000092.4 | MPP4/NM033066.2 | TBCEL/NM001130047.1 |
| COL5A1/NM000093.4 | MPP6/NM001303037.1 | TBCEL/NM152715.3 |
| COL5A2/NM000393.3 | MPP7/NM173496.4 | TBCK/NM001163435.2 |
| COL5A3/NM015719.3 | MPPE1/NM023075.5 | TBCK/NM001163436.2 |
| COL6A1/NM001848.2 | MPPED1/NM001044370.1 | TBK1/NM013254.3 |
| COL6A2/NM001849.3 | MPPED2/NM001584.2 | TBKBP1/NM014726.2 |
| COL6A3/NM004369.3 | MPRIP/NM201274.3 | TBL1XR1/NM001321193.1 |
| COL6A5/NM001278298.1 | MPST/NM001013436.2 | TBL1XR1/NM001321194.1 |
| COL6A6/NM001102608.1 | MPV17/NM002437.4 | TBL3/NM006453.2 |
| COL7A1/NM000094.3 | MPV17L/NM001128423.1 | TBP/NM003194.4 |
| COL8A2/NM005202.3 | MPV17L2/NM032683.2 | TBPL1/NM001253676.1 |
| COL9A1/NM001851.4 | MPZ/NM000530.7 | TBPL1/NM004865.3 |
| COL9A2/NM001852.3 | MPZL1/NM003953.5 | TBPL2/NM199047.2 |
| COL9A3/NM001853.3 | MPZL2/NM005797.3 | TBRG1/NM032811.2 |
| COLCA1/NM001302644.1 | MR1/NM001531.2 | TBRG4/NM004749.3 |
| COLCA1/NM001302645.1 | MRAP/NM178817.3 | TBX1/NM080647.1 |
| COLCA1/NM001302646.1 | MRAP2/NM138409.2 | TBX10/NM005995.4 |
| COLCA1/NM001302647.1 | MRAS/NM001085049.2 | TBX18/NM001080508.2 |
| COLCA1/NM207429.3 | MRAS/NM001252090.1 | TBX19/NM005149.2 |
| COLCA2/NM001271458.1 | MRAS/NM012219.4 | TBX2/NM005994.3 |
| COLEC10/NM006438.4 | MRC1/NM002438.3 | TBX20/NM001077653.2 |
| COLEC11/NM024027.4 | MRE11A/NM005591.3 | TBX21/NM013351.1 |
| COLEC12/NM130386.2 | MREG/NM018000.2 | TBX3/NM005996.3 |
| COLGALT1/NM024656.3 | MRFAP1/NM001272053.1 | TBX4/NM018488.3 |
| COLGALT2/NM015101.3 | MRFAP1L1/NM203462.2 | TBX5/NM000192.3 |
| COLQ/NM005677.3 | MRGBP/NM018270.5 | TBX5/NM181486.2 |
| COMMD1/NM152516.3 | MRGPRD/NM198923.2 | TBX6/NM004608.3 |
| COMMD10/NM016144.3 | MRGPRE/NM001039165.2 | TBXA2R/NM001060.5 |
| COMMD2/NM016094.3 | MRGPRF/NM001098515.1 | TBXAS1/NM001061.4 |
| COMMD3/NM012071.3 | MRGPRX1/NM147199.3 | TBXAS1/NM001130966.2 |
| COMMD4/NM017828.4 | MRGPRX2/NM001303615.1 | TC2N/NM001128596.2 |
| COMMD5/NM001081003.2 | MRGPRX2/NM054030.3 | TC2N/NM152332.5 |
| COMMD5/NM001287237.1 | MRGPRX3/NM054031.3 | TCAF1/NM014719.2 |
| COMMD7/NM053041.2 | MRGPRX4/NM054032.3 | TCAF2/NM001130026.2 |
| COMMD8/NM017845.3 | MRLN/NM001304732.1 | TCAIM/NM001282913.1 |
| COMMD9/NM014186.3 | MRM1/NM024864.4 | TCAIM/NM173826.3 |
| COMP/NM000095.2 | MRM2/NM013393.1 | TCAP/NM003673.3 |
| COMT/NM000754.3 | MRM3/NM018146.3 | TCEA1/NM006756.3 |
| COMT/NM001135161.1 | MROH1/NM032450.2 | TCEA2/NM003195.4 |
| COMT/NM001135162.1 | MROH2A/NM001287395.1 | TCEA3/NM003196.2 |
| COMTD1/NM144589.2 | MROH5/NM207414.2 | TCEANC2/NM153035.2 |
| COPA/NM004371.3 | MROH6/NM001100878.1 | TCEB1/NM001204858.1 |
| COPB1/NM001144061.1 | MROH7/NM001039464.3 | TCEB1/NM001204861.1 |
| COPB1/NM001144062.1 | MROH8/NM152503.5 | TCEB2/NM007108.3 |
| COPB1/NM016451.4 | MROH9/NM001163629.1 | TCEB3/NM003198.2 |
| COPB2/NM004766.2 | MRPL1/NM020236.3 | TCEB3B/NM016427.2 |
| COPE/NM007263.3 | MRPL10/NM145255.3 | TCEB3C/NM145653.3 |
| COPG1/NM016128.3 | MRPL11/NM016050.4 | TCEB3CL/NM001100817.1 |
| COPG2/NM012133.5 | MRPL12/NM002949.3 | TCEB3CL2/NM001242907.1 |
| COPRS/NM018405.3 | MRPL13/NM014078.5 | TCERG1/NM006706.3 |
| COPS2/NM004236.3 | MRPL14/NM001318768.1 | TCERG1L/NM174937.3 |
| COPS3/NM003653.3 | MRPL14/NM032111.3 | TCF12/NM207036.1 |
| COPS4/NM016129.2 | MRPL15/NM014175.3 | TCF15/NM004609.3 |
| COPS5/NM006837.2 | MRPL16/NM017840.3 | TCF20/NM005650.3 |
| COPS6/NM006833.4 | MRPL17/NM022061.3 | TCF21/NM003206.3 |
| COPS7A/NM001164093.1 | MRPL18/NM014161.4 | TCF21/NM198392.2 |
| COPS7A/NM016319.3 | MRPL19/NM014763.3 | TCF24/NM001193502.1 |
| COPS7B/NM022730.3 | MRPL2/NM015950.4 | TCF25/NM014972.2 |
| COPS9/NM001163424.1 | MRPL20/NM017971.3 | TCF3/NM003200.3 |
| COPZ1/NM016057.2 | MRPL22/NM014180.3 | TCF4/NM003199.2 |
| COPZ2/NM016429.3 | MRPL23/NM021134.3 | TCF7/NM003202.3 |
| COQ10A/NM144576.3 | MRPL24/NM145729.2 | TCF7L1/NM031283.2 |
| COQ10B/NM025147.4 | MRPL27/NM016504.2 | TCF7L2/NM030756.4 |
| COQ2/NM015697.7 | MRPL28/NM006428.4 | TCFL5/NM006602.3 |
| COQ3/NM017421.3 | MRPL3/NM007208.3 | TCHHL1/NM001008536.1 |
| COQ4/NM016035.4 | MRPL30/NM145212.3 | TCHP/NM001143852.1 |
| COQ5/NM032314.3 | MRPL32/NM031903.2 | TCHP/NM032300.4 |
| COQ6/NM182476.2 | MRPL33/NM004891.3 | TCL1A/NM021966.2 |
| COQ7/NM016138.4 | MRPL34/NM023937.3 | TCL1B/NM004918.3 |
| COQ9/NM020312.3 | MRPL35/NM016622.3 | TCN1/NM001062.3 |
| CORIN/NM006587.3 | MRPL36/NM032479.3 | TCN2/NM000355.3 |
| CORO1A/NM007074.3 | MRPL37/NM016491.3 | TCP1/NM030752.2 |
| CORO1B/NM001018070.2 | MRPL38/NM032478.3 | TCP10L/NM144659.5 |
| CORO1B/NM020441.2 | MRPL39/NM017446.3 | TCP11/NM018679.5 |
| CORO1C/NM001276471.1 | MRPL4/NM146387.1 | TCP11L1/NM001145541.1 |
| CORO1C/NM014325.3 | MRPL40/NM003776.3 | TCP11L1/NM018393.3 |
| CORO2A/NM003389.3 | MRPL41/NM032477.2 | TCP11L2/NM152772.2 |
| CORO2A/NM052820.3 | MRPL42/NM014050.3 | TCTA/NM022171.2 |
| CORO2B/NM006091.4 | MRPL43/NM032112.2 | TCTE1/NM182539.3 |
| CORO6/NM032854.3 | MRPL44/NM022915.3 | TCTE3/NM174910.1 |
| CORO7-PAM16/NM001201479.1 | MRPL45/NM032351.5 | TCTEX1D1/NM152665.2 |
| CORT/NM001302.4 | MRPL46/NM022163.3 | TCTEX1D2/NM152773.4 |
| COTL1/NM021149.3 | MRPL47/NM020409.2 | TCTEX1D4/NM001013632.3 |
| COX10/NM001303.3 | MRPL48/NM016055.5 | TCTN1/NM001082538.2 |
| COX11/NM004375.4 | MRPL49/NM004927.3 | TCTN2/NM024809.4 |
| COX14/NM032901.3 | MRPL50/NM019051.2 | TCTN3/NM015631.5 |
| COX18/NM173827.3 | MRPL51/NM016497.3 | TDG/NM003211.4 |
| COX19/NM001031617.2 | MRPL52/NM180982.2 | TDO2/NM005651.3 |
| COX20/NM001312871.1 | MRPL53/NM053050.4 | TDP1/NM001008744.1 |
| COX20/NM198076.5 | MRPL54/NM172251.2 | TDP2/NM016614.2 |
| COX4I1/NM001861.4 | MRPL55/NM181463.2 | TDRD1/NM198795.1 |
| COX4I2/NM032609.2 | MRPL57/NM024026.4 | TDRD10/NM182499.3 |
| COX5A/NM004255.3 | MRPL9/NM031420.3 | TDRD15/NM001306137.1 |
| COX6A1/NM004373.3 | MRPS10/NM018141.3 | TDRD3/NM001146070.1 |
| COX6B1/NM001863.4 | MRPS11/NM022839.4 | TDRD6/NM001010870.2 |
| COX6B2/NM144613.4 | MRPS12/NM033362.3 | TDRD7/NM014290.2 |
| COX6C/NM004374.3 | MRPS12/NM033363.1 | TDRD9/NM153046.2 |
| COX7A1/NM001864.3 | MRPS14/NM022100.2 | TDRKH/NM006862.3 |
| COX7A2/NM001865.3 | MRPS15/NM031280.3 | TDRP/NM175075.4 |
| COX7A2L/NM001319036.1 | MRPS16/NM016065.3 | TEAD1/NM021961.5 |
| COX7A2L/NM004718.3 | MRPS17/NM015969.2 | TEAD3/NM003214.3 |
| COX7C/NM001867.2 | MRPS18A/NM018135.3 | TEAD4/NM003213.3 |
| COX8A/NM004074.2 | MRPS18B/NM014046.3 | TEC/NM003215.2 |
| COX8C/NM182971.2 | MRPS18C/NM016067.3 | TECPR1/NM015395.2 |
| CP/NM000096.3 | MRPS2/NM016034.4 | TECPR2/NM014844.4 |
| CPA2/NM001869.2 | MRPS21/NM031901.5 | TECR/NM138501.5 |
| CPA3/NM001870.3 | MRPS22/NM020191.2 | TECTA/NM005422.2 |
| CPA5/NM001127441.1 | MRPS23/NM016070.3 | TECTB/NM058222.2 |
| CPA6/NM020361.4 | MRPS24/NM032014.2 | TEDDM1/NM172000.3 |
| CPAMD8/NM015692.2 | MRPS25/NM022497.4 | TEF/NM003216.3 |
| CPB1/NM001871.2 | MRPS26/NM030811.3 | TEFM/NM024683.3 |
| CPB2/NM001872.4 | MRPS27/NM015084.2 | TEK/NM000459.4 |
| CPE/NM001873.3 | MRPS28/NM014018.2 | TEKT1/NM053285.1 |
| CPEB1/NM001079533.1 | MRPS30/NM016640.3 | TEKT2/NM014466.2 |
| CPEB3/NM014912.4 | MRPS31/NM005830.3 | TEKT3/NM031898.2 |
| CPEB4/NM030627.3 | MRPS33/NM016071.3 | TEKT4/NM144705.3 |
| CPED1/NM024913.4 | MRPS33/NM053035.2 | TEKT5/NM144674.1 |
| CPLX1/NM006651.3 | MRPS34/NM023936.1 | TELO2/NM016111.3 |
| CPLX2/NM001008220.1 | MRPS35/NM021821.3 | TEN1/NM001113324.2 |
| CPLX2/NM006650.3 | MRPS36/NM033281.5 | TENM2/NM001122679.1 |
| CPLX3/NM001030005.2 | MRPS5/NM031902.4 | TENM3/NM001080477.3 |
| CPLX4/NM181654.3 | MRPS7/NM015971.3 | TEP1/NM007110.4 |
| CPM/NM001874.4 | MRPS9/NM182640.2 | TEPP/NM199046.2 |
| CPM/NM198320.3 | MRRF/NM138777.3 | TERB1/NM001136505.1 |
| CPN1/NM001308.2 | MRS2/NM020662.3 | TERF1/NM017489.2 |
| CPNE1/NM152927.2 | MRTO4/NM016183.3 | TERF2/NM005652.4 |
| CPNE2/NM152727.5 | MRVI1/NM001098579.2 | TERF2IP/NM018975.3 |
| CPNE3/NM003909.4 | MS4A1/NM021950.3 | TERT/NM198253.2 |
| CPNE4/NM130808.2 | MS4A10/NM206893.3 | TES/NM015641.3 |
| CPNE5/NM020939.1 | MS4A12/NM017716.2 | TESC/NM017899.3 |
| CPNE6/NM006032.3 | MS4A13/NM001012417.2 | TESK1/NM006285.2 |
| CPNE7/NM014427.4 | MS4A14/NM032597.4 | TESK2/NM007170.2 |
| CPNE8/NM153634.2 | MS4A2/NM000139.4 | TESMIN/NM004923.3 |
| CPNE9/NM153635.2 | MS4A3/NM006138.4 | TESPA1/NM001098815.2 |
| CPO/NM173077.2 | MS4A4A/NM148975.2 | TESPA1/NM001136030.2 |
| CPOX/NM000097.5 | MS4A5/NM023945.2 | TET1/NM030625.2 |
| CPPED1/NM018340.2 | MS4A6A/NM152852.2 | TET3/NM001287491.1 |
| CPQ/NM016134.3 | MS4A6E/NM139249.2 | TEX10/NM017746.3 |
| CPS1/NM001875.4 | MS4A7/NM206939.1 | TEX101/NM031451.4 |
| CPSF1/NM013291.2 | MS4A8/NM031457.1 | TEX12/NM031275.4 |
| CPSF2/NM001322272.1 | MSANTD2/NM001308027.1 | TEX14/NM198393.3 |
| CPSF2/NM017437.2 | MSANTD3-TMEFF1/NM001198812.1 | TEX19/NM207459.3 |
| CPSF3/NM016207.3 | MSANTD3/NM001198805.1 | TEX2/NM018469.4 |
| CPSF3L/NM017871.5 | MSANTD3/NM080655.2 | TEX22/NM001195082.1 |
| CPSF4/NM006693.3 | MSANTD4/NM032424.2 | TEX26/NM152325.1 |
| CPSF4L/NM001129885.1 | MSC/NM005098.3 | TEX261/NM144582.2 |
| CPSF6/NM007007.2 | MSGN1/NM001105569.1 | TEX264/NM001243726.1 |
| CPSF7/NM001136040.2 | MSH2/NM000251.2 | TEX264/NM015926.5 |
| CPT1A/NM001876.3 | MSH3/NM002439.4 | TEX29/NM152324.2 |
| CPT1B/NM001145137.1 | MSH4/NM002440.3 | TEX30/NM138779.4 |
| CPT1B/NM004377.3 | MSH5/NM172166.3 | TEX35/NM032126.4 |
| CPT1B/NM152245.2 | MSH6/NM000179.2 | TEX37/NM152670.2 |
| CPT1C/NM152359.2 | MSI1/NM002442.3 | TEX38/NM001145474.3 |
| CPT2/NM000098.2 | MSI2/NM138962.3 | TEX40/NM001039496.1 |
| CPTP/NM001029885.1 | MSL2/NM018133.3 | TEX43/NM207408.1 |
| CPVL/NM019029.2 | MSLN/NM013404.4 | TEX9/NM198524.2 |
| CPXM1/NM019609.4 | MSMB/NM002443.3 | TF/NM001063.3 |
| CPXM2/NM198148.2 | MSMP/NM001044264.2 | TFAM/NM003201.2 |
| CPZ/NM003652.3 | MSR1/NM138715.2 | TFAP2A/NM003220.2 |
| CR1/NM000651.4 | MSRA/NM012331.4 | TFAP2B/NM003221.3 |
| CR1L/NM175710.1 | MSRB1/NM016332.2 | TFAP2D/NM172238.3 |
| CR2/NM001877.4 | MSRB2/NM012228.3 | TFAP2E/NM178548.3 |
| CRABP1/NM004378.2 | MSRB3/NM198080.3 | TFAP4/NM003223.2 |
| CRABP2/NM001199723.1 | MSS51/NM001024593.1 | TFB1M/NM016020.3 |
| CRABP2/NM001878.3 | MST1L/NM001271733.1 | TFB2M/NM022366.2 |
| CRACR2A/NM001144958.1 | MST1R/NM002447.3 | TFCP2/NM005653.4 |
| CRACR2B/NM173584.4 | MSTN/NM005259.2 | TFCP2L1/NM014553.2 |
| CRADD/NM001320099.1 | MSTO1/NM018116.3 | TFDP1/NM007111.4 |
| CRADD/NM003805.4 | MSX1/NM002448.3 | TFDP2/NM001178138.1 |
| CRAMP1/NM020825.3 | MSX2/NM002449.4 | TFDP2/NM006286.4 |
| CRAT/NM000755.3 | MT1A/NM005946.2 | TFEB/NM001271944.1 |
| CRB1/NM201253.2 | MT1B/NM005947.2 | TFEB/NM001271945.1 |
| CRB2/NM173689.6 | MT1E/NM175617.3 | TFEB/NM007162.2 |
| CRB3/NM174881.3 | MT1F/NM005949.3 | TFEC/NM012252.3 |
| CRBN/NM016302.3 | MT1G/NM005950.2 | TFF1/NM003225.2 |
| CRCP/NM014478.4 | MT1H/NM005951.2 | TFF2/NM005423.4 |
| CRCT1/NM019060.2 | MT1HL1/NM001276687.1 | TFF3/NM003226.3 |
| CREB1/NM134442.4 | MT1M/NM176870.2 | TFG/NM001007565.2 |
| CREB3/NM006368.4 | MT2A/NM005953.3 | TFG/NM001195478.1 |
| CREB3L1/NM052854.3 | MT3/NM005954.2 | TFG/NM006070.5 |
| CREB3L2/NM194071.3 | MT4/NM032935.2 | TFIP11/NM001008697.1 |
| CREB3L3/NM032607.2 | MTA1/NM004689.3 | TFPI/NM006287.4 |
| CREB3L4/NM001255978.1 | MTA2/NM004739.3 | TFPT/NM013342.3 |
| CREB3L4/NM001255979.1 | MTA3/NM020744.3 | TFR2/NM003227.3 |
| CREB5/NM182898.3 | MTAP/NM002451.3 | TFRC/NM001128148.2 |
| CREBBP/NM004380.2 | MTBP/NM022045.4 | TFRC/NM003234.3 |
| CREBL2/NM001310.3 | MTCH1/NM014341.2 | TG/NM003235.4 |
| CREBRF/NM153607.2 | MTCH2/NM014342.3 | TGDS/NM014305.3 |
| CREBZF/NM001039618.2 | MTCL1/NM015210.3 | TGFA/NM003236.3 |
| CREG1/NM003851.2 | MTDH/NM178812.3 | TGFB1/NM000660.5 |
| CREG2/NM153836.3 | MTERF1/NM006980.4 | TGFB1I1/NM001164719.1 |
| CRELD1/NM001077415.2 | MTERF2/NM001033050.2 | TGFB1I1/NM015927.4 |
| CRELD1/NM015513.4 | MTERF3/NM015942.4 | TGFB3/NM003239.3 |
| CRELD2/NM024324.4 | MTERF4/NM182501.3 | TGFBI/NM000358.2 |
| CREM/NM181571.2 | MTF1/NM005955.2 | TGFBR1/NM004612.3 |
| CRH/NM000756.3 | MTF2/NM007358.3 | TGFBR2/NM003242.5 |
| CRHBP/NM001882.3 | MTFMT/NM139242.3 | TGFBR3/NM003243.4 |
| CRHR1/NM004382.4 | MTFP1/NM016498.4 | TGFBRAP1/NM004257.5 |
| CRHR2/NM001883.4 | MTFR1/NM014637.3 | TGIF1/NM001278684.1 |
| CRIM1/NM016441.2 | MTFR1L/NM001099625.1 | TGIF1/NM003244.3 |
| CRIP2/NM001312.3 | MTFR1L/NM001099626.1 | TGIF2-C20orf24/NM001199535.1 |
| CRIP3/NM206922.2 | MTFR1L/NM019557.5 | TGIF2/NM001199513.1 |
| CRIPAK/NM175918.3 | MTFR2/NM001099286.2 | TGIF2/NM021809.6 |
| CRIPT/NM014171.5 | MTG1/NM138384.2 | TGM1/NM000359.2 |
| CRISP1/NM001131.2 | MTG2/NM015666.3 | TGM2/NM001323316.1 |
| CRISP1/NM001205220.1 | MTHFD1/NM005956.3 | TGM2/NM004613.3 |
| CRISP2/NM003296.3 | MTHFD1L/NM015440.4 | TGM4/NM003241.3 |
| CRISP3/NM006061.2 | MTHFD2/NM006636.3 | TGM5/NM201631.3 |
| CRISPLD1/NM031461.5 | MTHFR/NM005957.4 | TGM6/NM198994.2 |
| CRISPLD2/NM031476.3 | MTHFS/NM006441.3 | TGM7/NM052955.2 |
| CRK/NM016823.3 | MTHFSD/NM001159377.1 | TGOLN2/NM006464.3 |
| CRKL/NM005207.3 | MTIF2/NM001005369.1 | TGS1/NM024831.7 |
| CRLF1/NM004750.4 | MTIF2/NM001321004.1 | TH/NM000360.3 |
| CRLF3/NM015986.3 | MTIF3/NM001166261.1 | THADA/NM001083953.1 |
| CRLS1/NM019095.5 | MTIF3/NM001166262.1 | THADA/NM022065.4 |
| CRMP1/NM001313.4 | MTIF3/NM001166263.1 | THAP1/NM018105.2 |
| CRNDE/NM001308963.1 | MTMR10/NM017762.2 | THAP10/NM020147.3 |
| CRNKL1/NM016652.5 | MTMR11/NM001145862.1 | THAP11/NM020457.2 |
| CRNN/NM016190.2 | MTMR12/NM001040446.2 | THAP12/NM004705.3 |
| CROCC/NM014675.4 | MTMR14/NM001077525.2 | THAP2/NM031435.3 |
| CROT/NM021151.3 | MTMR2/NM016156.5 | THAP3/NM138350.3 |
| CRTAC1/NM018058.6 | MTMR3/NM021090.3 | THAP4/NM015963.5 |
| CRTAM/NM019604.3 | MTMR4/NM004687.4 | THAP5/NM001130475.2 |
| CRTAP/NM006371.4 | MTMR6/NM004685.3 | THAP6/NM144721.5 |
| CRTC1/NM015321.2 | MTMR7/NM004686.4 | THAP7/NM030573.2 |
| CRTC2/NM181715.2 | MTMR9/NM015458.3 | THAP8/NM152658.2 |
| CRTC3/NM022769.4 | MTNR1A/NM005958.4 | THAP9/NM024672.5 |
| CRY1/NM004075.4 | MTNR1B/NM005959.3 | THBD/NM000361.2 |
| CRY2/NM021117.3 | MTO1/NM133645.2 | THBS1/NM003246.3 |
| CRYAA/NM000394.3 | MTOR/NM004958.3 | THBS2/NM003247.3 |
| CRYAB/NM001289808.1 | MTPAP/NM018109.3 | THBS3/NM007112.4 |
| CRYAB/NM001885.2 | MTR/NM000254.2 | THBS4/NM003248.5 |
| CRYBA1/NM005208.4 | MTRF1/NM004294.2 | THEG/NM016585.4 |
| CRYBA4/NM001886.2 | MTRF1L/NM019041.6 | THEG5/NM001205273.2 |
| CRYBB1/NM001887.3 | MTRNR2L1/NM001190452.1 | THEGL/NM001256475.1 |
| CRYBB2/NM000496.2 | MTRNR2L2/NM001190470.1 | THEM4/NM053055.4 |
| CRYBB3/NM004076.4 | MTRNR2L3/NM001190472.1 | THEM5/NM182578.3 |
| CRYBG3/NM153605.3 | MTRNR2L4/NM001190476.1 | THEM6/NM016647.2 |
| CRYGA/NM014617.3 | MTRNR2L5/NM001190478.1 | THEMIS/NM001010923.2 |
| CRYGB/NM005210.3 | MTRNR2L6/NM001190487.2 | THEMIS2/NM001105556.2 |
| CRYGC/NM020989.3 | MTRNR2L7/NM001190489.1 | THG1L/NM017872.4 |
| CRYGD/NM006891.3 | MTRNR2L8/NM001190702.1 | THNSL1/NM024838.4 |
| CRYGS/NM017541.2 | MTRNR2L9/NM001190706.1 | THNSL2/NM018271.4 |
| CRYL1/NM015974.2 | MTRR/NM024010.2 | THOC1/NM005131.2 |
| CRYM/NM001888.4 | MTSS1/NM014751.5 | THOC3/NM032361.3 |
| CRYZ/NM001130042.1 | MTSS1L/NM138383.2 | THOC5/NM001002877.1 |
| CRYZL1/NM145858.2 | MTTP/NM000253.3 | THOC5/NM001002878.1 |
| CS/NM004077.2 | MTURN/NM152793.2 | THOC6/NM024339.3 |
| CSAD/NM001244705.1 | MTUS1/NM001001931.2 | THOC7/NM025075.3 |
| CSDC2/NM014460.3 | MTUS2/NM015233.5 | THOP1/NM003249.4 |
| CSDE1/NM001007553.2 | MTX1/NM002455.4 | THPO/NM000460.3 |
| CSE1L/NM001316.3 | MTX3/NM001010891.4 | THPO/NM001289998.1 |
| CSF1/NM172211.3 | MUC1/NM002456.5 | THRA/NM199334.3 |
| CSF1R/NM001288705.1 | MUC12/NM001164462.1 | THRAP3/NM005119.3 |
| CSF1R/NM005211.3 | MUC13/NM033049.3 | THRB/NM001252634.1 |
| CSF2/NM000758.3 | MUC16/NM024690.2 | THRSP/NM003251.3 |
| CSF2RB/NM000395.2 | MUC17/NM001040105.1 | THSD1/NM018676.3 |
| CSF3/NM000759.3 | MUC2/NM002457.3 | THSD4/NM024817.2 |
| CSF3R/NM156039.3 | MUC20/NM001282506.1 | THSD7A/NM015204.2 |
| CSGALNACT1/NM001130518.1 | MUC21/NM001010909.3 | THSD7B/NM001316349.1 |
| CSGALNACT1/NM018371.4 | MUC22/NM001198815.1 | THTPA/NM001126339.3 |
| CSGALNACT2/NM018590.4 | MUC3A/NM005960.1 | THUMPD1/NM017736.4 |
| CSH2/NM020991.3 | MUC4/NM004532.5 | THUMPD2/NM025264.4 |
| CSK/NM001127190.1 | MUC5AC/NM001304359.1 | THUMPD3/NM001114092.1 |
| CSMD1/NM033225.5 | MUC5B/NM002458.2 | THY1/NM001311160.1 |
| CSN1S1/NM001890.1 | MUC6/NM005961.2 | THY1/NM001311162.1 |
| CSN2/NM001891.3 | MUC7/NM001145006.1 | THY1/NM006288.4 |
| CSN3/NM005212.2 | MUC7/NM001145007.1 | THYN1/NM014174.2 |
| CSNK1A1L/NM145203.5 | MUC7/NM152291.2 | TIAF1/NM004740.3 |
| CSNK1D/NM001893.4 | MUL1/NM024544.2 | TIAL1/NM003252.3 |
| CSNK1E/NM001894.4 | MUM1/NM032853.3 | TIAM1/NM003253.2 |
| CSNK1E/NM152221.2 | MURC/NM001018116.2 | TIAM2/NM012454.3 |
| CSNK1G1/NM022048.3 | MUS81/NM025128.4 | TICAM1/NM182919.3 |
| CSNK1G2/NM001319.6 | MUSK/NM005592.3 | TICAM2/NM021649.7 |
| CSNK1G3/NM004384.4 | MUSTNI/NM205853.3 | TICRR/NM152259.3 |
| CSNK2A1/NM177559.2 | MUT/NM000255.3 | TIE1/NM005424.4 |
| CSNK2A2/NM001896.2 | MUTYH/NM012222.2 | TIFA/NM052864.2 |
| CSNK2A3/NM001256686.1 | MVB12A/NM138401.3 | TIFAB/NM001099221.1 |
| CSNK2B/NM001320.6 | MVB12B/NM033446.2 | TIGAR/NM020375.2 |
| CSPG4/NM001897.4 | MVD/NM002461.2 | TIGD1/NM145702.2 |
| CSPG5/NM001206943.1 | MVP/NM005115.4 | TIGD2/NM145715.2 |
| CSPP1/NM024790.6 | MX1/NM001144925.2 | TIGD3/NM145719.2 |
| CSRNP1/NM033027.3 | MX1/NM001178046.2 | TIGD4/NM145720.3 |
| CSRNP2/NM030809.2 | MXD1/NM002357.3 | TIGD5/NM032862.4 |
| CSRNP3/NM001172173.1 | MXD3/NM031300.3 | TIGD6/NM001243253.1 |
| CSRNP3/NM024969.3 | MXD4/NM006454.2 | TIGD6/NM030953.3 |
| CSRP1/NM001193572.1 | MXI1/NM005962.4 | TIGIT/NM173799.3 |
| CSRP2/NM001321.2 | MXRA7/NM001008528.2 | TIMD4/NM138379.2 |
| CSRP3/NM003476.4 | MXRA8/NM001282582.1 | TIMELESS/NM003920.3 |
| CST1/NM001898.2 | MXRA8/NM032348.3 | TIMM10/NM012456.2 |
| CST11/NM130794.1 | MYADM/NM001020820.2 | TIMM10B/NM012192.3 |
| CST2/NM001322.2 | MYADM/NM001020821.2 | TIMM13/NM012458.3 |
| CST3/NM001288614.1 | MYADM/NM001290190.1 | TIMM17A/NM006335.2 |
| CST4/NM001899.2 | MYADM/NM001290193.1 | TIMM22/NM013337.2 |
| CST5/NM001900.4 | MYADM/NM001290194.1 | TIMM23/NM006327.3 |
| CST6/NM001323.3 | MYADML2/NM001145113.2 | TIMM23B/NM001290117.1 |
| CST7/NM003650.3 | MYB/NM005375.3 | TIMM50/NM001001563.2 |
| CST8/NM001281730.1 | MYBBP1A/NM014520.3 | TIMM8B/NM012459.2 |
| CST9/NM001008693.2 | MYBL1/NM001080416.3 | TIMM9/NM001304485.1 |
| CST9L/NM080610.2 | MYBL2/NM002466.3 | TIMM9/NM001304486.1 |
| CSTA/NM005213.3 | MYBPC1/NM002465.3 | TIMMDC1/NM016589.3 |
| CSTB/NM000100.3 | MYBPC2/NM004533.3 | TIMP2/NM003255.4 |
| CSTF1/NM001033521.1 | MYBPC3/NM000256.3 | TIMP3/NM000362.4 |
| CSTF1/NM001033522.1 | MYBPH/NM004997.2 | TIMP4/NM003256.3 |
| CSTF1/NM001324.2 | MYC/NM002467.4 | TINAG/NM014464.3 |
| CSTF2T/NM015235.2 | MYCBP/NM012333.4 | TINAGL1/NM022164.2 |
| CSTF3/NM001326.2 | MYCBP2/NM015057.4 | TINF2/NM001099274.1 |
| CSTL1/NM138283.1 | MYCBPAP/NM032133.4 | TIPARP/NM001184717.1 |
| CTAGE1/NM172241.2 | MYCL/NM001033081.2 | TIPARP/NM001184718.1 |
| CTAGE15/NM001008747.2 | MYCN/NM005378.5 | TIPARP/NM015508.4 |
| CTAGE4/NM198495.2 | MYCT1/NM025107.2 | TIPIN/NM017858.2 |
| CTAGE5/NM005930.3 | MYD88/NM002468.4 | TIPRL/NM152902.4 |
| CTAGE6/NM178561.4 | MYDGF/NM019107.3 | TIRAP/NM001039661.1 |
| CTAGE8/NM001278507.1 | MYEF2/NM016132.4 | TJAP1/NM001146020.1 |
| CTAGE9/NM001145659.1 | MYEOV/NM138768.3 | TJP1/NM003257.4 |
| CTBP1/NM001328.2 | MYF5/NM005593.2 | TJP3/NM001267560.1 |
| CTBP2/NM001083914.2 | MYF6/NM002469.2 | TK1/NM003258.4 |
| CTBP2/NM001290214.2 | MYH1/NM005963.3 | TK2/NM004614.4 |
| CTBP2/NM001321012.1 | MYH10/NM005964.3 | TKFC/NM015533.3 |
| CTBP2/NM001321014.1 | MYH11/NM002474.2 | TKT/NM001135055.2 |
| CTBS/NM004388.2 | MYH13/NM003802.2 | TKTL2/NM032136.4 |
| CTC1/NM025099.5 | MYH14/NM001077186.1 | TLCD1/NM138463.3 |
| CTCF/NM006565.3 | MYH15/NM014981.1 | TLCD2/NM001164407.1 |
| CTCFL/NM001269041.1 | MYH3/NM002470.3 | TLDC1/NM020947.3 |
| CTCFL/NM080618.3 | MYH4/NM017533.2 | TLDC2/NM080628.2 |
| CTDNEP1/NM001143775.1 | MYH6/NM002471.3 | TLE1/NM005077.4 |
| CTDNEP1/NM015343.4 | MYH7/NM000257.3 | TLE2/NM003260.4 |
| CTDP1/NM004715.4 | MYH7B/NM020884.4 | TLE3/NM005078.3 |
| CTDSP1/NM021198.2 | MYH8/NM002472.2 | TLE4/NM007005.4 |
| CTDSP2/NM005730.3 | MYH9/NM002473.5 | TLE6/NM024760.2 |
| CTDSPL/NM001008392.1 | MYL10/NM138403.4 | TLK1/NM012290.4 |
| CTDSPL2/NM016396.2 | MYL12A/NM001303048.1 | TLK2/NM006852.3 |
| CTF1/NM001330.3 | MYL12A/NM006471.3 | TLL1/NM012464.4 |
| CTGF/NM001901.2 | MYL12B/NM001144944.1 | TLN1/NM006289.3 |
| CTH/NM001902.5 | MYL12B/NM001144945.1 | TLN2/NM015059.2 |
| CTHRC1/NM138455.3 | MYL12B/NM033546.3 | TLR1/NM003263.3 |
| CTIF/NM014772.2 | MYL2/NM000432.3 | TLR10/NM030956.3 |
| CTLA4/NM005214.4 | MYL3/NM000258.2 | TLR2/NM001318793.1 |
| CTNNA1/NM001323983.1 | MYL4/NM001002841.1 | TLR3/NM003265.2 |
| CTNNA1/NM001903.4 | MYL4/NM002476.2 | TLR4/NM138554.4 |
| CTNNA3/NM001127384.2 | MYL5/NM002477.1 | TLR5/NM003268.5 |
| CTNNA3/NM013266.3 | MYL6/NM021019.4 | TLR6/NM006068.4 |
| CTNNAL1/NM003798.3 | MYL6B/NM002475.4 | TLR9/NM017442.3 |
| CTNNB1/NM001098210.1 | MYL7/NM021223.2 | TLX1/NM005521.3 |
| CTNNBIP1/NM020248.2 | MYL9/NM006097.4 | TLX2/NM016170.4 |
| CTNNBL1/NM030877.4 | MYLIP/NM013262.3 | TLX3/NM021025.3 |
| CTNND1/NM001085458.1 | MYLK/NM053027.3 | TM2D1/NM032027.2 |
| CTNND2/NM001332.3 | MYLK3/NM182493.2 | TM2D2/NM078473.2 |
| CTNS/NM004937.2 | MYLK4/NM001012418.3 | TM2D3/NM025141.3 |
| CTPS1/NM001905.3 | MYLPF/NM001324458.1 | TM4SF1/NM014220.2 |
| CTR9/NM014633.4 | MYLPF/NM001324459.1 | TM4SF18/NM001184723.1 |
| CTRB2/NM001025200.3 | MYLPF/NM013292.4 | TM4SF18/NM138786.3 |
| CTRC/NM007272.2 | MYNN/NM018657.4 | TM4SF19/NM138461.3 |
| CTRL/NM001907.2 | MYO10/NM012334.2 | TM4SF20/NM024795.3 |
| CTSB/NM147780.3 | MYO15A/NM016239.3 | TM4SF4/NM004617.3 |
| CTSD/NM001909.4 | MYO16/NM015011.1 | TM4SF5/NM003963.2 |
| CTSE/NM001910.3 | MYO18A/NM078471.3 | TM6SF1/NM023003.3 |
| CTSF/NM003793.3 | MYO18B/NM032608.6 | TM6SF2/NM001001524.2 |
| CTSG/NM001911.2 | MYO19/NM001163735.1 | TM7SF2/NM003273.3 |
| CTSH/NM004390.4 | MYO1A/NM001256041.1 | TM7SF3/NM016551.2 |
| CTSK/NM000396.3 | MYO1A/NM005379.3 | TM9SF1/NM006405.6 |
| CTSL/NM001257972.1 | MYO1B/NM012223.3 | TM9SF2/NM004800.2 |
| CTSO/NM001334.2 | MYO1C/NM033375.4 | TM9SF3/NM020123.3 |
| CTSS/NM004079.4 | MYO1D/NM015194.2 | TMA16/NM018352.2 |
| CTSV/NM001201575.1 | MYO1E/NM004998.3 | TMA7/NM015933.4 |
| CTSV/NM001333.3 | MYO1F/NM012335.3 | TMBIM1/NM001321427.1 |
| CTSW/NM001335.3 | MYO1G/NM033054.2 | TMBIM1/NM001321429.1 |
| CTSZ/NM001336.3 | MYO1H/NM001101421.3 | TMBIM1/NM001321430.1 |
| CTTN/NM138565.2 | MYO3A/NM017433.4 | TMBIM1/NM001321436.1 |
| CTTNBP2/NM033427.2 | MYO5A/NM000259.3 | TMBIM4/NM016056.3 |
| CTTNBP2NL/NM018704.2 | MYO5B/NM001080467.2 | TMBIM6/NM003217.2 |
| CTU1/NM145232.3 | MYO5C/NM018728.3 | TMC1/NM138691.2 |
| CTU2/NM001012762.2 | MYO6/NM001300899.1 | TMC2/NM080751.2 |
| CTXN1/NM206833.3 | MYO7A/NM001127180.1 | TMC3/NM001080532.1 |
| CTXN2/NM001145668.1 | MYO7B/NM001080527.1 | TMC4/NM144686.3 |
| CTXN3/NM001048252.2 | MYO9A/NM006901.3 | TMC5/NM024780.4 |
| CTXN3/NM001127385.1 | MYO9B/NM001130065.1 | TMC6/NM001127198.2 |
| CUBN/NM001081.3 | MYOC/NM000261.1 | TMC6/NM007267.7 |
| CUEDC1/NM001271875.1 | MYOCD/NM153604.3 | TMC7/NM024847.4 |
| CUEDC1/NM001292025.1 | MYOD1/NM002478.4 | TMC8/NM152468.4 |
| CUEDC2/NM024040.2 | MYOF/NM013451.3 | TMCC1/NM001017395.3 |
| CUL1/NM003592.2 | MYOG/NM002479.5 | TMCC2/NM001297613.1 |
| CUL2/NM001198777.1 | MYOM1/NM003803.3 | TMCC3/NM020698.3 |
| CUL2/NM003591.3 | MYOM2/NM003970.3 | TMCO1/NM019026.4 |
| CUL3/NM003590.4 | MYOM3/NM152372.3 | TMCO2/NM001008740.3 |
| CUL4A/NM001008895.2 | MYOT/NM006790.2 | TMCO3/NM017905.4 |
| CUL5/NM003478.3 | MYOZ1/NM021245.3 | TMCO4/NM181719.4 |
| CUL7/NM014780.4 | MYOZ2/NM016599.4 | TMED1/NM006858.3 |
| CUL9/NM015089.3 | MYOZ3/NM133371.4 | TMED10/NM006827.5 |
| CUTA/NM015921.2 | MYPN/NM032578.3 | TMED2/NM006815.3 |
| CUX1/NM181552.3 | MYPOP/NM001012643.3 | TMED3/NM007364.3 |
| CUX2/NM015267.3 | MYRF/NM013279.3 | TMED4/NM182547.3 |
| CUZD1/NM022034.5 | MYRFL/NM182530.2 | TMED5/NM016040.4 |
| CWC15/NM016403.3 | MYRIP/NM001284423.1 | TMED6/NM144676.3 |
| CWC22/NM020943.2 | MYRIP/NM015460.3 | TMED8/NM213601.1 |
| CWC25/NM017748.4 | MYSM1/NM001085487.2 | TMED9/NM017510.4 |
| CWC27/NM005869.3 | MYT1/NM004535.2 | TMEFF1/NM003692.4 |
| CWF19L1/NM018294.5 | MYZAP/NM001018100.4 | TMEFF2/NM016192.3 |
| CWF19L2/NM152434.2 | MZB1/NM016459.3 | TMEM100/NM001099640.1 |
| CWH43/NM025087.2 | MZF1/NM198055.1 | TMEM100/NM018286.2 |
| CX3CL1/NM002996.4 | MZT1/NM001071775.2 | TMEM101/NM032376.3 |
| CX3CR1/NM001171171.1 | MZT2A/NM001085365.1 | TMEM102/NM001320444.1 |
| CX3CR1/NM001171172.1 | MZT2B/NM025029.3 | TMEM102/NM178518.2 |
| CX3CR1/NM001337.3 | N4BP1/NM153029.3 | TMEM104/NM017728.3 |
| CXADR/NM001338.4 | N4BP2/NM018177.5 | TMEM106A/NM145041.3 |
| CXCL1/NM001511.3 | N4BP2L1/NM052818.2 | TMEM106B/NM001134232.1 |
| CXCL10/NM001565.3 | N4BP2L2/NM014887.2 | TMEM106C/NM024056.3 |
| CXCL11/NM005409.4 | N4BP3/NM015111.1 | TMEM107/NM183065.2 |
| CXCL12/NM199168.3 | N6AMT1/NM013240.5 | TMEM108/NM001136469.2 |
| CXCL13/NM006419.2 | NAA11/NM032693.2 | TMEM109/NM024092.2 |
| CXCL14/NM004887.4 | NAA15/NM057175.3 | TMEM11/NM003876.2 |
| CXCL16/NM022059.3 | NAA16/NM024561.4 | TMEM110-MUSTN1/NM001198974.2 |
| CXCL17/NM198477.2 | NAA25/NM024953.3 | TMEM114/NM001146336.1 |
| CXCL2/NM002089.3 | NAA30/NM001011713.2 | TMEM115/NM007024.4 |
| CXCL3/NM002090.2 | NAA35/NM001321881.1 | TMEM116/NM001294314.1 |
| CXCL5/NM002994.4 | NAA35/NM001321882.1 | TMEM117/NM032256.2 |
| CXCL6/NM002993.3 | NAA38/NM001320925.1 | TMEM119/NM181724.2 |
| CXCL8/NM000584.3 | NAA40/NM024771.3 | TMEM120A/NM031925.2 |
| CXCL9/NM002416.2 | NAA50/NM025146.3 | TMEM120B/NM001080825.2 |
| CXCR1/NM000634.2 | NAA60/NM001083600.2 | TMEM121/NM025268.2 |
| CXCR2/NM001168298.1 | NAA60/NM001083601.2 | TMEM123/NM052932.2 |
| CXCR2/NM001557.3 | NAAA/NM014435.3 | TMEM125/NM144626.2 |
| CXCR4/NM001008540.1 | NAALAD2/NM005467.3 | TMEM127/NM017849.3 |
| CXCR5/NM001716.4 | NAALADL1/NM005468.2 | TMEM128/NM001297551.1 |
| CXCR6/NM006564.1 | NAALADL2/NM207015.2 | TMEM129/NM001127266.1 |
| CXXC1/NM014593.3 | NAB1/NM005966.3 | TMEM130/NM152913.2 |
| CXXC4/NM025212.3 | NAB2/NM005967.3 | TMEM131/NM015348.1 |
| CXXC5/NM001317200.1 | NABP1/NM001031716.2 | TMEM132A/NM017870.3 |
| CXXC5/NM001317201.1 | NABP2/NM024068.3 | TMEM132B/NM052907.3 |
| CXXC5/NM001317202.1 | NACA/NM001113201.2 | TMEM132C/NM001136103.2 |
| CXXC5/NM001317205.1 | NACA/NM001320193.1 | TMEM132D/NM133448.2 |
| CXXC5/NM001317207.1 | NACA2/NM199290.3 | TMEM132E/NM001304438.1 |
| CXXC5/NM001317211.1 | NACAD/NM001146334.1 | TMEM133/NM032021.2 |
| CXXC5/NM016463.8 | NACC1/NM052876.3 | TMEM134/NM025124.3 |
| CYB561/NM001017916.1 | NACC2/NM144653.4 | TMEM135/NM022918.3 |
| CYB561/NM001017917.1 | NADK/NM001198993.1 | TMEM136/NM001198671.1 |
| CYB561/NM001915.3 | NADK/NM023018.4 | TMEM138/NM016464.4 |
| CYB561A3/NM001161452.1 | NADK2/NM001085411.2 | TMEM139/NM001282876.1 |
| CYB561A3/NM153611.4 | NADSYN1/NM018161.4 | TMEM139/NM153345.3 |
| CYB561D1/NM182580.2 | NAE1/NM003905.3 | TMEM140/NM018295.4 |
| CYB561D2/NM001291284.1 | NAF1/NM138386.2 | TMEM141/NM032928.3 |
| CYB5A/NM148923.3 | NAGA/NM000262.2 | TMEM143/NM018273.3 |
| CYB5B/NM030579.2 | NAGK/NM017567.4 | TMEM144/NM018342.4 |
| CYB5D1/NM144607.4 | NAGLU/NM000263.3 | TMEM147/NM032635.3 |
| CYB5D2/NM144611.3 | NAGPA/NM016256.3 | TMEM14A/NM014051.3 |
| CYB5R1/NM016243.2 | NAGS/NM153006.2 | TMEM14B/NM030969.4 |
| CYB5R2/NM001302826.1 | NAIF1/NM197956.3 | TMEM14C/NM016462.3 |
| CYB5R2/NM016229.4 | NAIP/NM004536.2 | TMEM150A/NM001031738.2 |
| CYB5R3/NM000398.6 | NALCN/NM052867.2 | TMEM150B/NM001282011.1 |
| CYB5R4/NM016230.3 | NAMPT/NM005746.2 | TMEM150C/NM001080506.1 |
| CYB5RL/NM001031672.2 | NANOGNB/NM001145465.1 | TMEM151A/NM153266.3 |
| CYBA/NM000101.3 | NANOS1/NM199461.3 | TMEM151B/NM001137560.1 |
| CYBRD1/NM024843.3 | NANOS2/NM001029861.2 | TMEM154/NM152680.2 |
| CYC1/NM001916.4 | NANOS3/NM001098622.2 | TMEM155/NM152399.3 |
| CYCS/NM018947.5 | NANP/NM152667.2 | TMEM156/NM024943.2 |
| CYFIP1/NM001287810.2 | NANS/NM018946.3 | TMEM158/NM015444.2 |
| CYFIP1/NM001324120.1 | NAP1L1/NM004537.5 | TMEM159/NM001301771.1 |
| CYFIP2/NM001037333.2 | NAP1L4/NM005969.3 | TMEM160/NM017854.1 |
| CYFIP2/NM014376.3 | NAP1L5/NM153757.3 | TMEM161A/NM017814.2 |
| CYGB/NM134268.4 | NAPA/NM003827.3 | TMEM163/NM030923.4 |
| CYHR1/NM138496.1 | NAPB/NM022080.2 | TMEM165/NM018475.4 |
| CYLC2/NM001340.4 | NAPEPLD/NM001122838.1 | TMEM167A/NM174909.4 |
| CYLD/NM001042355.1 | NAPG/NM003826.2 | TMEM167B/NM020141.3 |
| CYLD/NM001042412.1 | NAPRT/NM145201.5 | TMEM168/NM001287497.1 |
| CYP11A1/NM000781.2 | NAPSA/NM004851.2 | TMEM169/NM001142312.1 |
| CYP11B2/NM000498.3 | NARF/NM012336.3 | TMEM17/NM198276.2 |
| CYP17A1/NM000102.3 | NARFL/NM022493.2 | TMEM170A/NM145254.2 |
| CYP19A1/NM031226.2 | NARS/NM004539.3 | TMEM170B/NM001100829.2 |
| CYP1A1/NM001319217.1 | NARS2/NM024678.5 | TMEM171/NM173490.7 |
| CYP1A2/NM000761.4 | NASP/NM152298.3 | TMEM173/NM198282.3 |
| CYP1B1/NM000104.3 | NAT1/NM000662.7 | TMEM174/NM153217.2 |
| CYP20A1/NM177538.2 | NAT1/NM001160174.2 | TMEM175/NM032326.3 |
| CYP21A2/NM000500.7 | NAT1/NM001160179.2 | TMEM176A/NM018487.2 |
| CYP24A1/NM000782.4 | NAT10/NM024662.2 | TMEM176B/NM001101311.1 |
| CYP26B1/NM019885.3 | NAT14/NM020378.3 | TMEM176B/NM014020.3 |
| CYP26C1/NM183374.2 | NAT16/NM198571.2 | TMEM177/NM001105199.1 |
| CYP27A1/NM000784.3 | NAT2/NM000015.2 | TMEM178A/NM152390.2 |
| CYP27B1/NM000785.3 | NAT6/NM012191.3 | TMEM178B/NM001195278.1 |
| CYP2A13/NM000766.4 | NAT8/NM003960.3 | TMEM179/NM001286389.1 |
| CYP2A6/NM000762.5 | NAT8L/NM178557.3 | TMEM179B/NM199337.2 |
| CYP2A7/NM000764.2 | NAT9/NM015654.4 | TMEM18/NM152834.2 |
| CYP2B6/NM000767.4 | NATD1/NM152914.2 | TMEM181/NM020823.1 |
| CYP2C18/NM000772.2 | NAV1/NM020443.4 | TMEM182/NM144632.4 |
| CYP2C19/NM000769.2 | NAV2/NM001111018.1 | TMEM183A/NM138391.4 |
| CYP2C8/NM000770.3 | NAV3/NM014903.5 | TMEM183B/NM001079809.1 |
| CYP2D6/NM000106.5 | NAXD/NM001242882.1 | TMEM184A/NM001097620.1 |
| CYP2E1/NM000773.3 | NAXE/NM144772.2 | TMEM184B/NM001195071.1 |
| CYP2F1/NM000774.4 | NBAS/NM015909.3 | TMEM184B/NM012264.4 |
| CYP2J2/NM000775.3 | NBEA/NM015678.4 | TMEM184C/NM018241.2 |
| CYP2R1/NM024514.4 | NBEAL1/NM001114132.1 | TMEM185B/NM024121.2 |
| CYP2S1/NM030622.7 | NBEAL2/NM015175.2 | TMEM186/NM015421.3 |
| CYP2U1/NM183075.2 | NBL1/NM001204084.2 | TMEM189-UBE2V1/NM199203.2 |
| CYP2W1/NM017781.2 | NBL1/NM001204085.1 | TMEM19/NM018279.3 |
| CYP39A1/NM016593.4 | NBL1/NM001278164.1 | TMEM190/NM139172.1 |
| CYP3A4/NM017460.5 | NBL1/NM001278165.1 | TMEM191B/NM001242313.1 |
| CYP3A43/NM022820.4 | NBL1/NM001278166.1 | TMEM191C/NM001207052.1 |
| CYP3A5/NM000777.4 | NBL1/NM005380.7 | TMEM192/NM001100389.1 |
| CYP3A7-CYP3A51P/NM001256497.2 | NBN/NM002485.4 | TMEM198/NM001005209.2 |
| CYP46A1/NM006668.1 | NBPF1/NM017940.4 | TMEM198/NM001303098.1 |
| CYP4B1/NM000779.3 | NBPF10/NM001302371.1 | TMEM199/NM152464.2 |
| CYP4F11/NM021187.3 | NBPF11/NM001101663.4 | TMEM2/NM013390.2 |
| CYP4F12/NM023944.3 | NBPF12/NM001278141.1 | TMEM200A/NM001258276.1 |
| CYP4F2/NM001082.4 | NBPF14/NM015383.2 | TMEM200A/NM001258278.1 |
| CYP4F22/NM173483.3 | NBPF15/NM001170755.2 | TMEM200A/NM052913.2 |
| CYP4F3/NM000896.2 | NBPF15/NM173638.4 | TMEM200B/NM001003682.3 |
| CYP4F8/NM007253.3 | NBPF20/NM001278267.1 | TMEM200B/NM001171868.1 |
| CYP4V2/NM207352.3 | NBPF3/NM032264.4 | TMEM200C/NM001080209.1 |
| CYP4X1/NM178033.1 | NBPF4/NM001143989.2 | TMEM201/NM001010866.3 |
| CYP4Z1/NM178134.2 | NBPF6/NM001143988.1 | TMEM202/NM001080462.1 |
| CYP51A1/NM000786.3 | NBPF7/NM001047980.2 | TMEM203/NM053045.1 |
| CYP7A1/NM000780.3 | NBPF8/NM001037501.2 | TMEM204/NM024600.5 |
| CYP7B1/NM004820.3 | NBPF9/NM001277444.1 | TMEM205/NM198536.2 |
| CYP8B1/NM004391.2 | NBR1/NM005899.4 | TMEM206/NM018252.2 |
| CYR61/NM001554.4 | NBR1/NM031862.3 | TMEM207/NM207316.2 |
| CYS1/NM001037160.2 | NCALD/NM001040624.1 | TMEM208/NM014187.3 |
| CYSLTR2/NM001308471.1 | NCALD/NM001040630.1 | TMEM210/NM001282477.1 |
| CYSRT1/NM199001.3 | NCALD/NM032041.2 | TMEM211/NM001001663.1 |
| CYSTM1/NM032412.3 | NCAM1/NM181351.4 | TMEM213/NM001085429.1 |
| CYTH1/NM017456.3 | NCAM2/NM004540.3 | TMEM214/NM017727.4 |
| CYTH2/NM004228.6 | NCAPD2/NM014865.3 | TMEM215/NM212558.2 |
| CYTH3/NM004227.3 | NCAPD3/NM015261.2 | TMEM216/NM001173990.2 |
| CYTH4/NM013385.4 | NCAPG/NM022346.4 | TMEM217/NM145316.3 |
| CYTIP/NM004288.4 | NCAPG2/NM001281932.1 | TMEM218/NM001258238.1 |
| CYTL1/NM018659.2 | NCAPH/NM015341.4 | TMEM218/NM001258245.1 |
| CYYR1/NM052954.3 | NCAPH2/NM152299.3 | TMEM219/NM194280.3 |
| D2HGDH/NM152783.4 | NCBP1/NM002486.4 | TMEM221/NM001190844.1 |
| DAAM1/NM014992.2 | NCBP2/NM007362.3 | TMEM222/NM032125.2 |
| DAAM2/NM015345.3 | NCBP3/NM001114118.2 | TMEM223/NM001080501.2 |
| DAB1/NM021080.3 | NCCRP1/NM001001414.1 | TMEM225/NM001013743.2 |
| DAB2/NM001343.3 | NCDN/NM001014839.1 | TMEM229A/NM001136002.1 |
| DAB2IP/NM032552.3 | NCEH1/NM020792.4 | TMEM229B/NM182526.2 |
| DACH1/NM080759.5 | NCF1/NM000265.5 | TMEM230/NM014145.4 |
| DACT1/NM016651.5 | NCF2/NM000433.3 | TMEM231/NM001077418.2 |
| DACT2/NM214462.4 | NCF2/NM001127651.2 | TMEM232/NM001039763.3 |
| DACT3/NM145056.2 | NCF4/NM000631.4 | TMEM233/NM001136534.1 |
| DAD1/NM001344.3 | NCK1/NM006153.5 | TMEM234/NM019118.4 |
| DAG1/NM001177639.2 | NCK2/NM001004720.2 | TMEM235/NM001204210.1 |
| DAG1/NM001177643.2 | NCK2/NM003581.4 | TMEM236/NM001098844.2 |
| DAGLA/NM006133.2 | NCKAP1/NM205842.2 | TMEM237/NM001044385.2 |
| DAGLB/NM139179.3 | NCKAP5L/NM001037806.3 | TMEM238/NM001190764.1 |
| DALRD3/NM018114.5 | NCKIPSD/NM184231.2 | TMEM240/NM001114748.1 |
| DAND5/NM152654.2 | NCL/NM005381.2 | TMEM241/NM032933.5 |
| DAOA/NM172370.4 | NCLN/NM020170.3 | TMEM242/NM018452.5 |
| DAP/NM004394.2 | NCOA1/NM147223.2 | TMEM243/NM024315.2 |
| DAP3/NM001199849.1 | NCOA2/NM006540.3 | TMEM244/NM001010876.1 |
| DAP3/NM004632.3 | NCOA3/NM181659.2 | TMEM245/NM032012.3 |
| DAPK1/NM001288729.1 | NCOA4/NM001145260.1 | TMEM246/NM001303107.1 |
| DAPK1/NM001288730.1 | NCOA5/NM020967.2 | TMEM246/NM001303108.1 |
| DAPK1/NM001288731.1 | NCOA6/NM014071.4 | TMEM247/NM001145051.2 |
| DAPK2/NM014326.3 | NCOA7/NM001199622.1 | TMEM248/NM017994.4 |
| DAPK3/NM001348.2 | NCOR1/NM006311.3 | TMEM249/NM001252402.2 |
| DAPL1/NM001017920.2 | NCOR2/NM006312.5 | TMEM249/NM001280561.1 |
| DAPP1/NM014395.2 | NCR1/NM004829.6 | TMEM25/NM032780.3 |
| DARS/NM001349.3 | NCR2/NM004828.3 | TMEM252/NM153237.1 |
| DARS2/NM018122.4 | NCR3/NM147130.2 | TMEM253/NM001146683.1 |
| DAW1/NM178821.1 | NCR3LG1/NM001202439.2 | TMEM254/NM025125.3 |
| DAXX/NM001141969.1 | NCS1/NM014286.3 | TMEM255B/NM182614.2 |
| DAZAP1/NM018959.3 | NCSTN/NM015331.2 | TMEM256/NM152766.3 |
| DAZL/NM001351.3 | NDC1/NM018087.4 | TMEM259/NM033420.3 |
| DBF4/NM006716.3 | NDC80/NM006101.2 | TMEM260/NM017799.3 |
| DBF4B/NM145663.2 | NDEL1/NM030808.4 | TMEM261/NM033428.2 |
| DBH/NM000787.3 | NDFIP1/NM030571.3 | TMEM262/NM001282448.1 |
| DBI/NM001079862.2 | NDFIP2/NM019080.2 | TMEM263/NM001319661.1 |
| DBN1/NM080881.2 | NDN/NM002487.2 | TMEM263/NM001319662.1 |
| DBNDD1/NM001042610.2 | NDNF/NM024574.3 | TMEM265/NM001256829.1 |
| DBNDD2/NM001048221.2 | NDOR1/NM014434.3 | TMEM266/NM152335.2 |
| DBNDD2/NM001048223.2 | NDRG1/NM001135242.1 | TMEM30A/NM018247.3 |
| DBNDD2/NM001197139.1 | NDRG2/NM001320329.1 | TMEM30B/NM001017970.2 |
| DBNDD2/NM001197140.1 | NDRG2/NM201535.1 | TMEM33/NM018126.2 |
| DBNL/NM014063.6 | NDRG2/NM201540.1 | TMEM35B/NM001195156.1 |
| DBP/NM001352.4 | NDRG3/NM032013.3 | TMEM37/NM183240.2 |
| DBR1/NM016216.3 | NDRG4/NM001130487.1 | TMEM38A/NM024074.2 |
| DBX1/NM001029865.2 | NDST1/NM001543.4 | TMEM38B/NM018112.2 |
| DBX2/NM001004329.2 | NDST2/NM003635.3 | TMEM39A/NM018266.2 |
| DCAF10/NM024345.4 | NDST3/NM004784.2 | TMEM39B/NM018056.3 |
| DCAF11/NM001163484.1 | NDST4/NM022569.2 | TMEM40/NM018306.3 |
| DCAF11/NM025230.4 | NDUFA11/NM175614.4 | TMEM41A/NM080652.3 |
| DCAF12/NM015397.3 | NDUFA12/NM018838.4 | TMEM41B/NM015012.3 |
| DCAF13/NM015420.6 | NDUFA13/NM015965.6 | TMEM43/NM024334.2 |
| DCAF15/NM138353.2 | NDUFA2/NM002488.4 | TMEM44/NM138399.4 |
| DCAF16/NM017741.3 | NDUFA4/NM002489.3 | TMEM45A/NM018004.2 |
| DCAF17/NM025000.3 | NDUFA4L2/NM020142.3 | TMEM45B/NM138788.3 |
| DCAF4/NM015604.3 | NDUFA5/NM005000.4 | TMEM5/NM014254.2 |
| DCAF4L1/NM001029955.3 | NDUFA6/NM002490.3 | TMEM50A/NM014313.3 |
| DCAF4L2/NM152418.3 | NDUFA7/NM005001.4 | TMEM50B/NM006134.6 |
| DCAF5/NM003861.2 | NDUFA8/NM014222.2 | TMEM51/NM001136217.1 |
| DCAF6/NM018442.3 | NDUFA9/NM005002.4 | TMEM51/NM018022.2 |
| DCAF7/NM005828.4 | NDUFAB1/NM005003.2 | TMEM52/NM178545.3 |
| DCAF8/NM015726.3 | NDUFAF1/NM016013.3 | TMEM52B/NM153022.3 |
| DCAKD/NM001128631.2 | NDUFAF3/NM199069.1 | TMEM53/NM024587.3 |
| DCAKD/NM001288654.2 | NDUFAF4/NM014165.3 | TMEM54/NM033504.2 |
| DCAKD/NM001288655.1 | NDUFAF5/NM024120.4 | TMEM55A/NM018710.2 |
| DCANP1/NM130848.2 | NDUFAF6/NM152416.3 | TMEM55B/NM144568.3 |
| DCBLD1/NM173674.2 | NDUFAF7/NM144736.4 | TMEM56-RWDD3/NM001199691.1 |
| DCBLD2/NM080927.3 | NDUFB1/NM004545.3 | TMEM56/NM001199679.1 |
| DCD/NM001300854.1 | NDUFB10/NM004548.2 | TMEM56/NM152487.2 |
| DCDC1/NM181807.3 | NDUFB2/NM004546.2 | TMEM57/NM018202.5 |
| DCDC2/NM001195610.1 | NDUFB3/NM002491.2 | TMEM59/NM004872.4 |
| DCDC2/NM016356.4 | NDUFB4/NM004547.5 | TMEM59L/NM012109.2 |
| DCDC2B/NM001099434.1 | NDUFB5/NM002492.3 | TMEM60/NM032936.3 |
| DCDC2C/NM001287444.1 | NDUFB6/NM002493.4 | TMEM61/NM182532.2 |
| DCDC5/NM020869.3 | NDUFB7/NM004146.5 | TMEM62/NM024956.3 |
| DCHS1/NM003737.3 | NDUFB8/NM005004.3 | TMEM63A/NM014698.2 |
| DCLK1/NM004734.4 | NDUFC1/NM001184986.1 | TMEM63B/NM018426.2 |
| DCLK2/NM001040260.3 | NDUFC1/NM001184989.1 | TMEM63C/NM020431.3 |
| DCLK3/NM033403.1 | NDUFC1/NM002494.3 | TMEM64/NM001008495.3 |
| DCLRE1A/NM001271816.1 | NDUFC2-KCTD14/NM001203261.1 | TMEM65/NM194291.2 |
| DCLRE1A/NM014881.4 | NDUFS1/NM005006.6 | TMEM67/NM153704.5 |
| DCLRE1B/NM022836.3 | NDUFS2/NM004550.4 | TMEM68/NM152417.2 |
| DCLRE1C/NM001033855.2 | NDUFS3/NM004551.2 | TMEM69/NM016486.3 |
| DCN/NM001920.4 | NDUFS4/NM002495.3 | TMEM70/NM017866.5 |
| DCN/NM133503.3 | NDUFS5/NM001184979.1 | TMEM71/NM001145153.1 |
| DCP1A/NM018403.6 | NDUFS6/NM004553.4 | TMEM72/NM001123376.2 |
| DCP1B/NM152640.4 | NDUFS8/NM002496.3 | TMEM74/NM153015.2 |
| DCP2/NM152624.5 | NDUFV1/NM007103.3 | TMEM74B/NM001304749.1 |
| DCPS/NM014026.4 | NDUFV2/NM021074.4 | TMEM79/NM032323.2 |
| DCST1/NM152494.3 | NDUFV3/NM001001503.1 | TMEM80/NM001042463.2 |
| DCST2/NM144622.2 | NEB/NM001164507.1 | TMEM81/NM203376.1 |
| DCSTAMP/NM030788.3 | NEBL/NM213569.2 | TMEM82/NM001013641.2 |
| DCT/NM001922.4 | NECAB1/NM022351.4 | TMEM86A/NM153347.2 |
| DCTN1/NM004082.4 | NECAB2/NM019065.2 | TMEM86B/NM173804.4 |
| DCTN2/NM006400.4 | NECAB3/NM031232.3 | TMEM87B/NM032824.2 |
| DCTN3/NM007234.4 | NECAP1/NM015509.3 | TMEM88/NM203411.1 |
| DCTN5/NM032486.3 | NECAP2/NM018090.4 | TMEM88B/NM001146685.1 |
| DCTN6/NM006571.3 | NECTIN1/NM002855.4 | TMEM89/NM001008269.1 |
| DCTPP1/NM024096.1 | NECTIN2/NM002856.2 | TMEM8A/NM021259.2 |
| DCUN1D1/NM020640.3 | NECTIN3/NM015480.2 | TMEM8B/NM001042589.2 |
| DCUN1D2/NM001014283.1 | NECTIN4/NM030916.2 | TMEM8B/NM001042590.2 |
| DCUN1D3/NM173475.3 | NEDD4/NM006154.3 | TMEM9/NM001288564.1 |
| DCUN1D4/NM001040402.2 | NEDD4L/NM001144967.2 | TMEM9/NM001288565.1 |
| DCUN1D5/NM032299.3 | NEDD8-MDP1/NM001199823.1 | TMEM91/NM001098821.1 |
| DCXR/NM016286.3 | NEDD9/NM006403.3 | TMEM92/NM001168215.1 |
| DDA1/NM024050.5 | NEFH/NM021076.3 | TMEM92/NM153229.2 |
| DDAH1/NM012137.3 | NEFL/NM006158.4 | TMEM94/NM014738.5 |
| DDAH2/NM001303007.1 | NEFM/NM005382.2 | TMEM95/NM198154.2 |
| DDAH2/NM001303008.1 | NEGR1/NM173808.2 | TMEM97/NM014573.2 |
| DDB1/NM001923.4 | NEIL1/NM024608.3 | TMEM98/NM001033504.1 |
| DDB2/NM000107.2 | NEIL2/NM001135746.1 | TMEM99/NM001195386.1 |
| DDC/NM000790.3 | NEIL3/NM018248.2 | TMEM99/NM001195387.1 |
| DDC/NM001082971.1 | NEK1/NM012224.2 | TMEM99/NM145274.3 |
| DDHD1/NM030637.2 | NEK11/NM024800.4 | TMEM9B/NM020644.2 |
| DDHD2/NM001164232.1 | NEK2/NM002497.3 | TMF1/NM007114.2 |
| DDHD2/NM015214.2 | NEK3/NM002498.2 | TMIE/NM147196.2 |
| DDI1/NM001001711.2 | NEK3/NM152720.2 | TMIGD1/NM206832.2 |
| DDI2/NM032341.4 | NEK4/NM003157.4 | TMIGD2/NM144615.2 |
| DDIAS/NM145018.3 | NEK5/NM199289.2 | TMIGD3/NM020683.6 |
| DDIT3/NM004083.5 | NEK6/NM001166168.1 | TMOD1/NM001166116.1 |
| DDIT4/NM019058.2 | NEK6/NM001166170.1 | TMOD1/NM003275.3 |
| DDIT4L/NM145244.3 | NEK6/NM014397.5 | TMOD2/NM014548.3 |
| DDN/NM015086.1 | NEK7/NM133494.2 | TMOD3/NM014547.4 |
| DDO/NM003649.2 | NEK8/NM178170.2 | TMOD4/NM013353.2 |
| DDOST/NM005216.4 | NEK9/NM033116.4 | TMPO/NM003276.2 |
| DDR1/NM001297652.1 | NELFA/NM005663.4 | TMPPE/NM001039770.2 |
| DDR1/NM001297653.1 | NELFB/NM015456.4 | TMPRSS11A/NM182606.3 |
| DDR1/NM001954.4 | NELFCD/NM198976.2 | TMPRSS11B/NM182502.3 |
| DDR2/NM006182.2 | NELFE/NM002904.5 | TMPRSS11E/NM014058.3 |
| DDRGK1/NM023935.2 | NELL1/NM006157.4 | TMPRSS11F/NM207407.2 |
| DDT/NM001084392.1 | NELL2/NM001145108.1 | TMPRSS13/NM001077263.2 |
| DDT/NM001355.3 | NEMF/NM004713.4 | TMPRSS15/NM002772.2 |
| DDTL/NM001084393.1 | NEMP1/NM015257.2 | TMPRSS2/NM005656.3 |
| DDX10/NM004398.3 | NEMP2/NM001142645.1 | TMPRSS3/NM024022.2 |
| DDX11/NM030653.3 | NENF/NM013349.4 | TMPRSS4/NM019894.3 |
| DDX17/NM006386.4 | NEO1/NM002499.3 | TMPRSS5/NM030770.3 |
| DDX18/NM006773.3 | NEPRO/NM015412.3 | TMPRSS6/NM001289001.1 |
| DDX19A/NM018332.4 | NES/NM006617.1 | TMPRSS7/NM001042575.2 |
| DDX19B/NM007242.5 | NET1/NM005863.4 | TMPRSS9/NM182973.1 |
| DDX20/NM007204.4 | NETO1/NM001201465.1 | TMSB10/NM021103.3 |
| DDX21/NM004728.3 | NETO2/NM018092.4 | TMTC2/NM152588.2 |
| DDX23/NM004818.2 | NEU2/NM005383.2 | TMTC3/NM181783.3 |
| DDX24/NM020414.3 | NEU4/NM080741.3 | TMTC4/NM001079669.2 |
| DDX25/NM013264.4 | NEURL1/NM004210.4 | TMUB1/NM001136044.1 |
| DDX27/NM017895.7 | NEURL1B/NM001142651.2 | TMUB1/NM031434.3 |
| DDX28/NM018380.3 | NEURL3/NM001285485.1 | TMUB2/NM024107.2 |
| DDX31/NM022779.8 | NEURL4/NM032442.2 | TMUB2/NM177441.2 |
| DDX39A/NM005804.3 | NEUROD1/NM002500.4 | TMX1/NM030755.4 |
| DDX39B/NM004640.6 | NEUROD2/NM006160.3 | TMX2/NM015959.3 |
| DDX4/NM024415.2 | NEUROD4/NM021191.2 | TMX3/NM019022.3 |
| DDX41/NM016222.3 | NEUROG1/NM006161.2 | TMX4/NM021156.3 |
| DDX42/NM203499.2 | NEUROG2/NM024019.3 | TNC/NM002160.3 |
| DDX43/NM018665.2 | NEUROG3/NM020999.3 | TNF/NM000594.3 |
| DDX46/NM014829.3 | NEXN/NM144573.3 | TNFAIP1/NM021137.4 |
| DDX47/NM016355.3 | NF1/NM000267.3 | TNFAIP2/NM006291.2 |
| DDX49/NM019070.4 | NF2/NM000268.3 | TNFAIP3/NM006290.3 |
| DDX50/NM024045.1 | NFAM1/NM145912.6 | TNFAIP6/NM007115.3 |
| DDX51/NM175066.3 | NFASC/NM001005388.2 | TNFAIP8/NM001286813.1 |
| DDX52/NM007010.4 | NFAT5/NM006599.3 | TNFAIP8/NM014350.3 |
| DDX54/NM024072.3 | NFATC1/NM006162.4 | TNFAIP8L1/NM001167942.1 |
| DDX55/NM020936.2 | NFATC2/NM173091.3 | TNFAIP8L1/NM152362.2 |
| DDX56/NM019082.3 | NFATC2IP/NM032815.3 | TNFAIP8L2/NM024575.4 |
| DDX58/NM014314.3 | NFATC3/NM173165.2 | TNFAIP8L3/NM001311175.1 |
| DDX59/NM001031725.5 | NFATC4/NM004554.4 | TNFRSF10A/NM003844.3 |
| DDX60/NM017631.5 | NFE2/NM001261461.1 | TNFRSF10B/NM003842.4 |
| DDX60L/NM001012967.2 | NFE2/NM006163.2 | TNFRSF10C/NM003841.3 |
| DEAF1/NM021008.3 | NFE2L1/NM003204.2 | TNFRSF10D/NM003840.4 |
| DEC1/NM017418.2 | NFE2L2/NM006164.4 | TNFRSF11A/NM003839.3 |
| DECR1/NM001359.1 | NFE2L3/NM004289.6 | TNFRSF11B/NM002546.3 |
| DECR2/NM020664.3 | NFE4/NM001085386.1 | TNFRSF12A/NM016639.2 |
| DEDD/NM001039712.1 | NFIA/NM005595.4 | TNFRSF13B/NM012452.2 |
| DEDD/NM032998.2 | NFIC/NM005597.3 | TNFRSF13C/NM052945.3 |
| DEDD2/NM001270614.1 | NFIL3/NM001289999.1 | TNFRSF14/NM003820.3 |
| DEDD2/NM133328.3 | NFIL3/NM005384.2 | TNFRSF17/NM001192.2 |
| DEF6/NM022047.3 | NFIX/NM001271043.2 | TNFRSF18/NM148901.1 |
| DEF8/NM001242818.1 | NFKB1/NM003998.3 | TNFRSF19/NM018647.3 |
| DEFA1/NM004084.3 | NFKB2/NM001077494.3 | TNFRSF1A/NM001065.3 |
| DEFA1B/NM001042500.1 | NFKBIA/NM020529.2 | TNFRSF1B/NM001066.2 |
| DEFA3/NM005217.3 | NFKBID/NM001321831.1 | TNFRSF21/NM014452.4 |
| DEFA4/NM001925.2 | NFKBIE/NM004556.2 | TNFRSF25/NM148965.1 |
| DEFA5/NM021010.2 | NFKBIL1/NM005007.3 | TNFRSF4/NM003327.3 |
| DEFA6/NM001926.3 | NFKBIZ/NM031419.3 | TNFRSF6B/NM003823.3 |
| DEFB1/NM005218.3 | NFRKB/NM001143835.1 | TNFRSF8/NM001243.4 |
| DEFB103A/NM001081551.3 | NFS1/NM021100.4 | TNFRSF9/NM001561.5 |
| DEFB103B/NM018661.4 | NFU1/NM001002755.2 | TNFSF10/NM003810.3 |
| DEFB104A/NM080389.2 | NFX1/NM002504.5 | TNFSF11/NM003701.3 |
| DEFB104B/NM001040702.1 | NFXL1/NM001278623.1 | TNFSF12/NM003809.2 |
| DEFB105A/NM152250.2 | NFXL1/NM001278624.1 | TNFSF13/NM003808.3 |
| DEFB105B/NM001040703.2 | NFXL1/NM152995.5 | TNFSF13B/NM006573.4 |
| DEFB106A/NM152251.3 | NFYC/NM014223.4 | TNFSF14/NM003807.4 |
| DEFB106B/NM001040704.1 | NGB/NM021257.3 | TNFSF15/NM005118.3 |
| DEFB107A/NM001037668.1 | NGEF/NM001114090.1 | TNFSF18/NM005092.3 |
| DEFB107B/NM001040705.1 | NGF/NM002506.2 | TNFSF4/NM003326.4 |
| DEFB108B/NM001002035.1 | NGFR/NM002507.3 | TNFSF8/NM001244.3 |
| DEFB110/NM001037497.1 | NGLY1/NM018297.3 | TNFSF9/NM003811.3 |
| DEFB112/NM001037498.1 | NGRN/NM001033088.1 | TNIK/NM015028.3 |
| DEFB113/NM001037729.1 | NHEJ1/NM024782.2 | TNIP1/NM001252390.1 |
| DEFB114/NM001037499.1 | NHLH1/NM005598.3 | TNIP1/NM001252391.1 |
| DEFB115/NM001037730.1 | NHLH2/NM001111061.1 | TNIP1/NM001258454.1 |
| DEFB116/NM001037731.1 | NHLH2/NM005599.3 | TNIP1/NM006058.4 |
| DEFB118/NM054112.2 | NHLRC1/NM198586.2 | TNIP2/NM024309.3 |
| DEFB121/NM001011878.2 | NHLRC2/NM198514.3 | TNIP3/NM024873.5 |
| DEFB123/NM153324.3 | NHLRC3/NM001012754.3 | TNK1/NM003985.4 |
| DEFB124/NM001037500.1 | NHLRC4/NM176677.2 | TNK2/NM005781.4 |
| DEFB125/NM153325.3 | NHP2/NM017838.3 | TNKS/NM003747.2 |
| DEFB126/NM030931.3 | NHSL1/NM001144060.1 | TNKS1BP1/NM033396.2 |
| DEFB127/NM139074.3 | NID1/NM002508.2 | TNKS2/NM025235.3 |
| DEFB128/NM001037732.2 | NID2/NM007361.3 | TNN/NM022093.1 |
| DEFB129/NM080831.3 | NIF3L1/NM001136039.2 | TNNC2/NM003279.2 |
| DEFB130/NM001037804.1 | NIFK/NM032390.4 | TNNI2/NM001145829.1 |
| DEFB131/NM001040448.2 | NIM1K/NM153361.3 | TNNI2/NM003282.3 |
| DEFB132/NM207469.2 | NIN/NM182946.1 | TNNI3/NM000363.4 |
| DEFB133/NM001166478.1 | NINJ1/NM004148.3 | TNNI3K/NM015978.2 |
| DEFB134/NM001302695.1 | NINJ2/NM016533.5 | TNNT1/NM003283.5 |
| DEFB135/NM001033017.2 | NINL/NM025176.5 | TNNT3/NM006757.3 |
| DEFB136/NM001033018.2 | NIP7/NM016101.4 | TNP1/NM003284.3 |
| DEFB4A/NM004942.3 | NIPA1/NM144599.4 | TNP2/NM005425.4 |
| DEFB4B/NM001205266.1 | NIPA2/NM001008892.2 | TNPO1/NM153188.2 |
| DEGS1/NM003676.3 | NIPAL1/NM207330.2 | TNPO2/NM001136196.1 |
| DEGS2/NM206918.2 | NIPAL2/NM001321635.1 | TNPO3/NM012470.3 |
| DEK/NM003472.3 | NIPAL3/NM001322854.1 | TNR/NM003285.2 |
| DENND1A/NM020946.1 | NIPAL4/NM001099287.1 | TNRC18/NM001080495.2 |
| DENND1B/NM144977.4 | NIPBL/NM133433.3 | TNRC6A/NM014494.2 |
| DENND1C/NM024898.3 | NIPSNAP1/NM003634.3 | TNRC6B/NM015088.2 |
| DENND2A/NM015689.4 | NIPSNAP3A/NM015469.1 | TNRC6C/NM001142640.1 |
| DENND2C/NM198459.3 | NIPSNAP3B/NM018376.3 | TNS1/NM022648.5 |
| DENND2D/NM001271833.1 | NIT2/NM020202.4 | TNS2/NM015319.2 |
| DENND3/NM014957.2 | NKAIN1/NM024522.2 | TNS4/NM032865.5 |
| DENND4A/NM005848.4 | NKAIN2/NM001040214.2 | TNXB/NM019105.6 |
| DENND4B/NM014856.2 | NKAIN4/NM152864.3 | TOB1/NM001243877.1 |
| DENND4C/NM017925.6 | NKAPL/NM001007531.2 | TOB1/NM005749.3 |
| DENND5A/NM015213.3 | NKD1/NM033119.4 | TOB2/NM016272.3 |
| DENND5B/NM144973.3 | NKD2/NM033120.3 | TOE1/NM025077.3 |
| DENND6A/NM152678.2 | NKG7/NM005601.3 | TOLLIP/NM019009.3 |
| DENND6B/NM001001794.3 | NKIRAS1/NM020345.3 | TOM1/NM005488.2 |
| DENR/NM003677.4 | NKIRAS2/NM001001349.2 | TOM1L1/NM005486.2 |
| DEPDC1/NM017779.5 | NKIRAS2/NM017595.5 | TOM1L2/NM001082968.1 |
| DEPDC1B/NM018369.2 | NKPD1/NM198478.3 | TOMM20/NM014765.2 |
| DEPDC4/NM152317.3 | NKTR/NM005385.3 | TOMM20L/NM207377.2 |
| DEPDC5/NM014662.4 | NKX1-1/NM001290079.1 | TOMM22/NM020243.4 |
| DEPDC7/NM001077242.1 | NKX1-2/NM001146340.1 | TOMM34/NM006809.4 |
| DEPTOR/NM022783.3 | NKX2-2/NM002509.3 | TOMM40/NM006114.2 |
| DERA/NM015954.3 | NKX2-3/NM145285.2 | TOMM40L/NM032174.5 |
| DERL1/NM024295.5 | NKX2-4/NM033176.1 | TOMM5/NM001001790.2 |
| DERL2/NM016041.4 | NKX2-6/NM001136271.2 | TOMM6/NM001134493.1 |
| DERL3/NM001002862.2 | NKX2-8/NM014360.3 | TOMM70/NM014820.4 |
| DES/NM001927.3 | NKX3-1/NM006167.3 | TONSL/NM013432.4 |
| DESI1/NM015704.2 | NKX3-2/NM001189.3 | TOP1/NM003286.2 |
| DESI2/NM016076.4 | NKX6-1/NM006168.2 | TOP1MT/NM052963.2 |
| DET1/NM001144074.2 | NKX6-2/NM177400.2 | TOP2A/NM001067.3 |
| DET1/NM001321596.1 | NLE1/NM018096.4 | TOP2B/NM001068.3 |
| DEXI/NM014015.3 | NLGN1/NM014932.3 | TOP3A/NM004618.4 |
| DFFA/NM004401.2 | NLGN2/NM020795.3 | TOP3B/NM003935.4 |
| DFFB/NM004402.3 | NLK/NM016231.4 | TOPAZ1/NM001145030.1 |
| DFNA5/NM001127453.1 | NLN/NM020726.4 | TOPBP1/NM007027.3 |
| DFNA5/NM004403.2 | NLRC3/NM178844.3 | TOPORS/NM005802.4 |
| DFNB59/NM001042702.3 | NLRC4/NM001199138.1 | TOR1A/NM000113.2 |
| DGAT1/NM012079.5 | NLRC4/NM001199139.1 | TOR1AIP1/NM015602.3 |
| DGAT2/NM032564.4 | NLRC4/NM021209.4 | TOR1AIP2/NM001199260.1 |
| DGCR14/NM022719.2 | NLRC5/NM032206.4 | TOR1B/NM014506.2 |
| DGCR2/NM005137.2 | NLRP1/NM033004.3 | TOR2A/NM001085347.2 |
| DGCR6/NM005675.4 | NLRP12/NM144687.3 | TOR3A/NM022371.3 |
| DGCR6L/NM033257.3 | NLRP13/NM176810.2 | TOR4A/NM017723.2 |
| DGCR8/NM022720.6 | NLRP14/NM176822.3 | TOX/NM014729.2 |
| DGKA/NM001345.4 | NLRP2/NM001174081.1 | TOX2/NM001098796.1 |
| DGKA/NM201554.1 | NLRP2/NM017852.3 | TOX3/NM001080430.3 |
| DGKB/NM004080.2 | NLRP3/NM001079821.2 | TOX4/NM014828.3 |
| DGKD/NM152879.2 | NLRP3/NM004895.4 | TP53/NM000546.5 |
| DGKE/NM003647.2 | NLRP5/NM153447.4 | TP53BP1/NM005657.2 |
| DGKG/NM001346.2 | NLRP6/NM138329.2 | TP53BP2/NM001031685.2 |
| DGKH/NM178009.4 | NLRP7/NM139176.3 | TP53I11/NM001258320.1 |
| DGKI/NM004717.3 | NLRP8/NM176811.2 | TP53I11/NM001258322.1 |
| DGKQ/NM001347.3 | NLRP9/NM176820.3 | TP53I11/NM001258323.2 |
| DGKZ/NM201533.3 | NLRX1/NM001282143.1 | TP53I13/NM138349.2 |
| DGUOK/NM080916.2 | NLRX1/NM001282358.1 | TP53I3/NM004881.4 |
| DHCR24/NM014762.3 | NMB/NM021077.3 | TP53INP1/NM033285.3 |
| DHCR7/NM001360.2 | NMBR/NM002511.3 | TP53INP2/NM021202.1 |
| DHDDS/NM024887.3 | NMD3/NM015938.4 | TP53RK/NM033550.3 |
| DHDH/NM014475.3 | NME1-NME2/NM001018136.2 | TP53TG3/NM016212.4 |
| DHFR/NM000791.3 | NME1/NM000269.2 | TP53TG3B/NM001099687.3 |
| DHFRL1/NM001195643.1 | NME2/NM001018137.2 | TP53TG3C/NM001205259.2 |
| DHFRL1/NM176815.4 | NME2/NM001018138.1 | TP53TG3D/NM001243722.2 |
| DHH/NM021044.3 | NME2/NM001018139.2 | TP53TG5/NM014477.2 |
| DHODH/NM001361.4 | NME2/NM002512.3 | TP63/NM003722.4 |
| DHPS/NM001930.3 | NME3/NM002513.2 | TP73/NM005427.3 |
| DHRS1/NM001136050.2 | NME4/NM005009.2 | TPBG/NM001166392.1 |
| DHRS1/NM138452.2 | NME5/NM003551.2 | TPBG/NM006670.4 |
| DHRS11/NM024308.3 | NME6/NM005793.4 | TPBGL/NM001195528.1 |
| DHRS12/NM001270424.1 | NME7/NM013330.4 | TPCN1/NM017901.5 |
| DHRS13/NM144683.3 | NME8/NM016616.4 | TPCN2/NM139075.3 |
| DHRS3/NM004753.6 | NME9/NM178130.3 | TPD52/NM001025252.2 |
| DHRS4/NM021004.3 | NMI/NM004688.2 | TPD52L1/NM003287.3 |
| DHRS4L2/NM198083.3 | NMNAT1/NM001297778.1 | TPD52L2/NM199361.2 |
| DHRS7/NM016029.3 | NMNAT1/NM022787.3 | TPGS1/NM033513.2 |
| DHRS7B/NM015510.4 | NMNAT2/NM015039.3 | TPGS2/NM015476.3 |
| DHRS7C/NM001220493.1 | NMNAT3/NM001320510.1 | TPH1/NM004179.2 |
| DHRS9/NM001142270.1 | NMRAL1/NM001305141.1 | TPH2/NM173353.3 |
| DHRS9/NM001142271.1 | NMRAL1/NM020677.4 | TPI1/NM000365.5 |
| DHRS9/NM199204.1 | NMRK1/NM017881.2 | TPM1/NM000366.5 |
| DHTKD1/NM018706.6 | NMRK2/NM170678.2 | TPM2/NM003289.3 |
| DHX15/NM001358.2 | NMS/NM001011717.1 | TPM3/NM152263.3 |
| DHX16/NM003587.4 | NMT1/NM021079.4 | TPM4/NM001145160.1 |
| DHX29/NM019030.2 | NMT2/NM004808.2 | TPMT/NM000367.3 |
| DHX30/NM138615.2 | NMU/NM006681.3 | TPP1/NM000391.3 |
| DHX32/NM018180.2 | NMUR1/NM006056.4 | TPP2/NM003291.2 |
| DHX33/NM020162.3 | NMUR2/NM020167.4 | TPPP/NM007030.2 |
| DHX34/NM014681.5 | NNAT/NM005386.3 | TPPP2/NM173846.4 |
| DHX35/NM021931.3 | NNMT/NM006169.2 | TPPP3/NM016140.3 |
| DHX36/NM020865.2 | NNT/NM012343.3 | TPR/NM003292.2 |
| DHX37/NM032656.3 | NNT/NM182977.2 | TPRA1/NM001136053.2 |
| DHX38/NM014003.3 | NOA1/NM032313.3 | TPRG1/NM198485.3 |
| DHX40/NM024612.4 | NOB1/NM014062.2 | TPRG1L/NM182752.3 |
| DHX57/NM198963.1 | NOC2L/NM015658.3 | TPRKB/NM016058.2 |
| DHX58/NM024119.2 | NOC3L/NM022451.10 | TPRN/NM001128228.2 |
| DHX8/NM004941.2 | NOC4L/NM024078.2 | TPRX1/NM198479.2 |
| DHX9/NM001357.4 | NOCT/NM012118.3 | TPSAB1/NM003294.3 |
| DIABLO/NM019887.5 | NOD1/NM006092.2 | TPSB2/NM024164.5 |
| DIAPH1/NM005219.4 | NOD2/NM022162.2 | TPSD1/NM012217.2 |
| DIAPH3/NM001042517.1 | NODAL/NM018055.4 | TPSG1/NM012467.3 |
| DICER1/NM001271282.2 | NOG/NM005450.4 | TPST1/NM003596.3 |
| DICER1/NM030621.4 | NOL10/NM024894.3 | TPST2/NM001008566.1 |
| DICER1/NM177438.2 | NOL11/NM015462.4 | TPST2/NM003595.3 |
| DIDO1/NM001193370.1 | NOL12/NM024313.2 | TPT1/NM003295.3 |
| DIDO1/NM080797.3 | NOL3/NM001276309.1 | TPTE/NM199261.3 |
| DIEXF/NM014388.6 | NOL3/NM003946.6 | TPTE2/NM130785.3 |
| DIO1/NM000792.6 | NOL4/NM003787.4 | TPX2/NM012112.4 |
| DIO2/NM000793.5 | NOL4L/NM001256798.1 | TRA2A/NM013293.4 |
| DIO2/NM013989.4 | NOL6/NM022917.4 | TRA2B/NM004593.2 |
| DIO3/NM001362.3 | NOL8/NM017948.5 | TRABD/NM001320485.1 |
| DIP2A/NM015151.3 | NOL9/NM024654.4 | TRABD/NM001320487.1 |
| DIP2B/NM173602.2 | NOLC1/NM004741.4 | TRABD2A/NM001080824.2 |
| DIP2C/NM014974.2 | NOM1/NM138400.1 | TRABD2B/NM001194986.1 |
| DIRAS1/NM145173.3 | NOMO1/NM014287.3 | TRADD/NM001323552.1 |
| DIRAS2/NM017594.4 | NOMO2/NM173614.2 | TRAF1/NM001190945.1 |
| DIRAS3/NM004675.3 | NOMO3/NM001004067.3 | TRAF1/NM005658.4 |
| DIRC1/NM052952.2 | NOP10/NM018648.3 | TRAF2/NM021138.3 |
| DIRC2/NM032839.2 | NOP14/NM001291978.1 | TRAF3/NM003300.3 |
| DIS3/NM014953.4 | NOP16/NM016391.7 | TRAF3IP1/NM015650.3 |
| DIS3L/NM001143688.2 | NOP2/NM001033714.2 | TRAF3IP2/NM147686.3 |
| DIS3L2/NM152383.4 | NOP56/NM006392.3 | TRAF3IP3/NM025228.3 |
| DISC1/NM001012959.1 | NOP58/NM015934.4 | TRAF4/NM004295.3 |
| DISP1/NM032890.3 | NOP9/NM174913.2 | TRAF5/NM145759.2 |
| DISP2/NM033510.2 | NOS1/NM000620.4 | TRAF6/NM145803.2 |
| DISP3/NM020780.1 | NOS1AP/NM014697.2 | TRAF7/NM032271.2 |
| DIXDC1/NM001037954.3 | NOS2/NM000625.4 | TRAIP/NM005879.2 |
| DKK1/NM012242.2 | NOS3/NM000603.4 | TRAK1/NM001042646.2 |
| DKK2/NM014421.2 | NOSIP/NM015953.4 | TRAK2/NM015049.2 |
| DKK3/NM001018057.1 | NOSTRIN/NM001039724.3 | TRAM1/NM014294.5 |
| DKK3/NM013253.4 | NOTCH1/NM017617.4 | TRAMIL1/NM152402.2 |
| DKK3/NM015881.5 | NOTCH2/NM024408.3 | TRAM2/NM012288.3 |
| DKK4/NM014420.2 | NOTCH2NL/NM203458.4 | TRAP1/NM016292.2 |
| DLC1/NM182643.2 | NOTCH3/NM000435.2 | TRAPPC1/NM001166621.1 |
| DLD/NM000108.4 | NOTCH4/NM004557.3 | TRAPPC1/NM021210.4 |
| DLEC1/NM007335.3 | NOTO/NM001134462.1 | TRAPPC10/NM003274.4 |
| DLEU7/NM001306135.1 | NOTUM/NM178493.5 | TRAPPC11/NM021942.5 |
| DLG1/NM004087.2 | NOV/NM002514.3 | TRAPPC12/NM016030.5 |
| DLG4/NM001128827.2 | NOVA1/NM002515.2 | TRAPPC13/NM001093755.1 |
| DLG5/NM004747.3 | NOVA2/NM002516.3 | TRAPPC2L/NM016209.4 |
| DLGAP1/NM001242766.1 | NOX3/NM015718.2 | TRAPPC3/NM014408.4 |
| DLGAP2/NM004745.4 | NOX4/NM016931.4 | TRAPPC3L/NM001139444.2 |
| DLGAP3/NM001080418.2 | NOX5/NM001184779.1 | TRAPPC4/NM016146.5 |
| DLGAP4/NM014902.5 | NOXA1/NM006647.1 | TRAPPC5/NM001042462.1 |
| DLGAP5/NM014750.4 | NOXO1/NM172167.2 | TRAPPC6A/NM001270891.1 |
| DLK1/NM003836.6 | NOXRED1/NM001113475.2 | TRAPPC6B/NM001079537.1 |
| DLK2/NM023932.3 | NPAP1/NM018958.2 | TRAPPC8/NM014939.3 |
| DLK2/NM206539.2 | NPAS1/NM002517.3 | TRAPPC9/NM001160372.3 |
| DLL1/NM005618.3 | NPAS2/NM002518.3 | TRAT1/NM016388.3 |
| DLL3/NM203486.2 | NPAS3/NM022123.2 | TRDMT1/NM004412.6 |
| DLL4/NM019074.3 | NPAS4/NM178864.3 | TREH/NM007180.2 |
| DLST/NM001933.4 | NPAT/NM002519.2 | TREM1/NM018643.4 |
| DLX1/NM178120.4 | NPB/NM148896.4 | TREM2/NM018965.3 |
| DLX2/NM004405.3 | NPBWR1/NM005285.3 | TREML1/NM178174.3 |
| DLX3/NM005220.2 | NPBWR2/NM005286.2 | TREML2/NM024807.3 |
| DLX4/NM138281.2 | NPC1/NM000271.4 | TREML4/NM198153.2 |
| DLX5/NM005221.5 | NPC1L1/NM013389.2 | TRERF1/NM033502.3 |
| DLX6/NM005222.3 | NPC2/NM006432.3 | TREX1/NM033629.4 |
| DMAP1/NM001034024.1 | NPDC1/NM015392.3 | TRH/NM007117.4 |
| DMBT1/NM007329.2 | NPEPL1/NM024663.3 | TRHDE/NM013381.2 |
| DMBX1/NM172225.1 | NPEPPS/NM006310.3 | TRHR/NM003301.5 |
| DMC1/NM007068.3 | NPFF/NM003717.3 | TRIAP1/NM016399.2 |
| DMGDH/NM013391.3 | NPFFR1/NM022146.4 | TRIB1/NM025195.3 |
| DMKN/NM033317.4 | NPFFR2/NM004885.2 | TRIB2/NM021643.3 |
| DMP1/NM004407.3 | NPHP1/NM000272.3 | TRIB3/NM001301188.1 |
| DMPK/NM004409.4 | NPHP3/NM153240.4 | TRIB3/NM001301190.1 |
| DMRT1/NM021951.2 | NPHP4/NM015102.4 | TRIB3/NM001301196.1 |
| DMRT2/NM181872.4 | NPHS1/NM004646.3 | TRIB3/NM021158.4 |
| DMRT3/NM021240.3 | NPHS2/NM014625.3 | TRIL/NM014817.3 |
| DMRTA1/NM022160.2 | NPIPA1/NM006985.2 | TRIM10/NM006778.3 |
| DMRTA2/NM032110.2 | NPIPA7/NM001282507.1 | TRIM11/NM145214.2 |
| DMRTB1/NM033067.2 | NPIPA8/NM001282511.1 | TRIM13/NM005798.4 |
| DMRTC2/NM001040283.2 | NPIPB11/NM001310137.1 | TRIM14/NM014788.3 |
| DMTF1/NM001142327.1 | NPIPB15/NM001306094.1 | TRIM15/NM033229.2 |
| DMTF1/NM021145.3 | NPIPB3/NM130464.2 | TRIM16/NM006470.3 |
| DMTN/NM001114135.4 | NPIPB4/NM001310148.1 | TRIM16L/NM001037330.1 |
| DMTN/NM001114136.2 | NPIPB5/NM001135865.1 | TRIM17/NM016102.3 |
| DMTN/NM001978.4 | NPIPB6/NM001282524.1 | TRIM2/NM015271.4 |
| DMWD/NM004943.1 | NPIPB8/NM001310136.1 | TRIM21/NM003141.3 |
| DMXL1/NM005509.5 | NPIPB9/NM001287250.1 | TRIM22/NM006074.4 |
| DMXL2/NM015263.3 | NPL/NM030769.2 | TRIM23/NM001656.3 |
| DNA2/NM001080449.2 | NPLOC4/NM017921.3 | TRIM24/NM015905.2 |
| DNAAF1/NM178452.5 | NPM1/NM002520.6 | TRIM25/NM005082.4 |
| DNAAF2/NM018139.2 | NPM2/NM001286680.1 | TRIM26/NM003449.4 |
| DNAAF3/NM178837.4 | NPM2/NM182795.1 | TRIM27/NM006510.4 |
| DNAAF5/NM017802.3 | NPM3/NM006993.2 | TRIM28/NM005762.2 |
| DNAH1/NM015512.4 | NPNT/NM001033047.2 | TRIM3/NM001248006.1 |
| DNAH10/NM207437.3 | NPPA/NM006172.3 | TRIM3/NM006458.3 |
| DNAH11/NM001277115.1 | NPPB/NM002521.2 | TRIM31/NM007028.4 |
| DNAH14/NM001373.1 | NPPC/NM024409.3 | TRIM32/NM001099679.1 |
| DNAH17/NM173628.3 | NPR1/NM000906.3 | TRIM33/NM015906.3 |
| DNAH2/NM020877.3 | NPR2/NM003995.3 | TRIM34/NM001003827.1 |
| DNAH5/NM001369.2 | NPR3/NM000908.3 | TRIM34/NM021616.5 |
| DNAH6/NM001370.1 | NPRL2/NM006545.4 | TRIM35/NM171982.4 |
| DNAH7/NM018897.2 | NPS/NM001030013.1 | TRIM36/NM018700.3 |
| DNAH8/NM001206927.1 | NPTN/NM012428.3 | TRIM37/NM001005207.3 |
| DNAH9/NM001372.3 | NPTX1/NM002522.3 | TRIM38/NM006355.4 |
| DNAI1/NM012144.3 | NPTX2/NM002523.2 | TRIM39-RPP21/NM001199119.1 |
| DNAI2/NM023036.4 | NPTXR/NM014293.3 | TRIM39/NM172016.2 |
| DNAJA1/NM001539.3 | NPVF/NM022150.3 | TRIM4/NM033017.3 |
| DNAJA2/NM005880.3 | NPW/NM001099456.2 | TRIM40/NM138700.4 |
| DNAJA3/NM005147.5 | NPY2R/NM000910.3 | TRIM41/NM033549.4 |
| DNAJA4/NM018602.3 | NPY4R/NM005972.5 | TRIM42/NM152616.4 |
| DNAJB1/NM006145.2 | NPY5R/NM006174.3 | TRIM43/NM138800.1 |
| DNAJB11/NM016306.5 | NQO1/NM000903.2 | TRIM43B/NM001164464.1 |
| DNAJB12/NM001002762.2 | NQO2/NM000904.4 | TRIM44/NM017583.5 |
| DNAJB13/NM153614.3 | NQO2/NM001318940.1 | TRIM45/NM025188.3 |
| DNAJB14/NM001031723.3 | NR0B2/NM021969.2 | TRIM46/NM025058.4 |
| DNAJB2/NM006736.5 | NR1D1/NM021724.4 | TRIM47/NM033452.2 |
| DNAJB3/NM001001394.3 | NR1D2/NM005126.4 | TRIM48/NM024114.3 |
| DNAJB4/NM001317099.1 | NR1H2/NM007121.5 | TRIM49/NM020358.2 |
| DNAJB4/NM007034.4 | NR1H3/NM005693.3 | TRIM49B/NM001206626.1 |
| DNAJB5/NM001135004.2 | NR1H4/NM005123.3 | TRIM49C/NM001195234.1 |
| DNAJB7/NM145174.1 | NR1I2/NM022002.2 | TRIM49D1/NM001206627.1 |
| DNAJB8/NM153330.4 | NR1I3/NM005122.4 | TRIM49D2/NM001105522.1 |
| DNAJB9/NM012328.2 | NR2C1/NM003297.3 | TRIM5/NM033034.2 |
| DNAJC1/NM022365.3 | NR2C2/NM001291694.1 | TRIM50/NM001281451.1 |
| DNAJC10/NM018981.2 | NR2C2AP/NM176880.5 | TRIM50/NM178125.3 |
| DNAJC11/NM018198.3 | NR2E1/NM003269.4 | TRIM51/NM032681.3 |
| DNAJC12/NM021800.2 | NR2E3/NM014249.3 | TRIM52/NM032765.2 |
| DNAJC13/NM015268.3 | NR2F1/NM005654.5 | TRIM54/NM187841.2 |
| DNAJC14/NM032364.5 | NR2F2/NM021005.3 | TRIM56/NM030961.2 |
| DNAJC15/NM013238.2 | NR2F6/NM005234.3 | TRIM58/NM015431.3 |
| DNAJC16/NM015291.3 | NR3C1/NM000176.2 | TRIM59/NM173084.2 |
| DNAJC17/NM018163.2 | NR3C1/NM001018076.1 | TRIM6-TRIM34/NM001003819.3 |
| DNAJC18/NM152686.3 | NR3C2/NM000901.4 | TRIM60/NM152620.2 |
| DNAJC19/NM145261.3 | NR4A1/NM173157.2 | TRIM61/NM001012414.2 |
| DNAJC21/NM001012339.2 | NR4A2/NM006186.3 | TRIM62/NM018207.2 |
| DNAJC22/NM024902.3 | NR4A3/NM173200.2 | TRIM63/NM032588.3 |
| DNAJC24/NM181706.4 | NR5A1/NM004959.4 | TRIM64/NM001136486.1 |
| DNAJC25/NM001015882.2 | NR5A2/NM205860.2 | TRIM64B/NM001164397.1 |
| DNAJC27/NM016544.2 | NRARP/NM001004354.2 | TRIM64C/NM001206631.1 |
| DNAJC28/NM001040192.2 | NRAS/NM002524.4 | TRIM65/NM173547.3 |
| DNAJC28/NM017833.4 | NRBF2/NM030759.4 | TRIM67/NM001004342.3 |
| DNAJC3/NM006260.4 | NRBP1/NM013392.3 | TRIM68/NM018073.7 |
| DNAJC30/NM032317.2 | NRBP2/NM178564.3 | TRIM69/NM182985.4 |
| DNAJC4/NM005528.3 | NRCAM/NM001037132.2 | TRIM7/NM203293.2 |
| DNAJC5/NM025219.2 | NRDE2/NM017970.3 | TRIM71/NM001039111.2 |
| DNAJC5G/NM173650.2 | NREP/NM001142476.1 | TRIM73/NM198924.3 |
| DNAJC6/NM014787.3 | NREP/NM001142477.1 | TRIM74/NM198853.2 |
| DNAJC7/NM003315.3 | NREP/NM001142478.1 | TRIM77/NM001146162.1 |
| DNAJC8/NM014280.2 | NREP/NM001142479.1 | TRIM8/NM030912.2 |
| DNAJC9/NM015190.4 | NREP/NM001142481.1 | TRIM9/NM015163.5 |
| DNAL1/NM031427.3 | NREP/NM001142482.1 | TRIML1/NM178556.3 |
| DNAL4/NM005740.2 | NREP/NM001142483.1 | TRIML2/NM173553.2 |
| DNALI1/NM003462.3 | NREP/NM004772.2 | TRIO/NM007118.3 |
| DNASE1/NM005223.3 | NRF1/NM001040110.1 | TRIOBP/NM138632.2 |
| DNASE1L2/NM001301680.1 | NRG1/NM013956.4 | TRIP10/NM004240.3 |
| DNASE1L3/NM004944.3 | NRG3/NM001010848.3 | TRIP11/NM004239.4 |
| DNASE2/NM001375.2 | NRG4/NM138573.3 | TRIP12/NM001284215.1 |
| DNASE2B/NM021233.2 | NRGN/NM006176.2 | TRIP13/NM004237.3 |
| DND1/NM194249.2 | NRIP1/NM003489.3 | TRIP4/NM016213.4 |
| DNER/NM139072.3 | NRIP2/NM031474.2 | TRIP6/NM003302.2 |
| DNHD1/NM144666.2 | NRIP3/NM020645.2 | TRIQK/NM001171796.1 |
| DNLZ/NM001080849.2 | NRM/NM007243.2 | TRIQK/NM001191035.1 |
| DNM1/NM004408.3 | NRN1/NM001278710.1 | TRIQK/NM001191036.1 |
| DNM2/NM001005362.2 | NRN1/NM016588.2 | TRIT1/NM017646.5 |
| DNM3/NM001136127.2 | NRN1L/NM198443.1 | TRMO/NM016481.4 |
| DNMBP/NM015221.3 | NRP1/NM003873.5 | TRMT1/NM001136035.2 |
| DNMT3A/NM022552.4 | NRP2/NM201266.1 | TRMT1/NM017722.3 |
| DNMT3A/NM175629.2 | NRSN2/NM001323685.1 | TRMT10A/NM001134665.2 |
| DNMT3B/NM006892.3 | NRSN2/NM024958.2 | TRMT10A/NM152292.4 |
| DNMT3L/NM013369.3 | NRTN/NM004558.3 | TRMT10B/NM144964.3 |
| DNPEP/NM001319118.1 | NRXN1/NM004801.4 | TRMT10C/NM017819.3 |
| DNPEP/NM001319119.1 | NRXN2/NM015080.3 | TRMT112/NM016404.2 |
| DNPH1/NM006443.2 | NSA2/NM014886.4 | TRMT12/NM017956.3 |
| DNTT/NM004088.3 | NSD1/NM022455.4 | TRMT13/NM019083.2 |
| DNTTIP1/NM052951.2 | NSF/NM006178.3 | TRMT1L/NM030934.4 |
| DNTTIP2/NM014597.4 | NSFL1C/NM016143.4 | TRMT44/NM152544.2 |
| DOC2A/NM001282062.1 | NSG1/NM001040101.1 | TRMT5/NM020810.3 |
| DOC2A/NM001282063.1 | NSG1/NM014392.4 | TRMT6/NM015939.4 |
| DOC2A/NM001282068.1 | NSL1/NM015471.3 | TRMT61A/NM152307.2 |
| DOC2A/NM003586.2 | NSMAF/NM003580.3 | TRMT61B/NM017910.3 |
| DOC2B/NM003585.4 | NSMCE1/NM145080.3 | TRNAU1AP/NM017846.4 |
| DOCK1/NM001380.4 | NSMCE2/NM173685.2 | TRNP1/NM001013642.2 |
| DOCK10/NM001290263.1 | NSMCE3/NM138704.3 | TRNT1/NM182916.2 |
| DOCK2/NM004946.2 | NSMCE4A/NM017615.2 | TROAP/NM005480.3 |
| DOCK3/NM004947.4 | NSMF/NM015537.4 | TROVE2/NM001173524.1 |
| DOCK4/NM014705.3 | NSRP1/NM032141.3 | TROVE2/NM004600.5 |
| DOCK5/NM024940.7 | NSUN2/NM017755.5 | TRPA1/NM007332.2 |
| DOCK6/NM020812.3 | NSUN3/NM022072.3 | TRPC1/NM003304.4 |
| DOCK7/NM033407.3 | NSUN4/NM199044.3 | TRPC3/NM003305.2 |
| DOCK8/NM203447.3 | NSUN5/NM148956.3 | TRPC6/NM004621.5 |
| DOHH/NM001145165.1 | NSUN6/NM182543.3 | TRPC7/NM020389.2 |
| DOHH/NM031304.4 | NSUN7/NM024677.4 | TRPM1/NM002420.5 |
| DOK1/NM001381.4 | NT5C/NM014595.2 | TRPM2/NM003307.3 |
| DOK2/NM003974.3 | NT5C1A/NM032526.2 | TRPM3/NM206946.3 |
| DOK3/NM024872.2 | NT5C1B-RDH14/NM001199104.1 | TRPM4/NM017636.3 |
| DOK4/NM018110.3 | NT5C2/NM012229.4 | TRPM5/NM014555.3 |
| DOK5/NM018431.4 | NT5C3A/NM016489.12 | TRPM6/NM017662.4 |
| DOK6/NM152721.5 | NT5C3B/NM052935.4 | TRPM7/NM017672.5 |
| DOK7/NM173660.4 | NT5DC1/NM152729.2 | TRPM8/NM024080.4 |
| DOLK/NM014908.3 | NT5DC2/NM022908.2 | TRPS1/NM014112.4 |
| DONSON/NM017613.3 | NT5DC3/NM001031701.2 | TRPT1/NM001033678.3 |
| DOPEY2/NM001320714.1 | NT5E/NM002526.3 | TRPV1/NM018727.5 |
| DOPEY2/NM005128.3 | NT5M/NM020201.3 | TRPV1/NM080704.3 |
| DOT1L/NM032482.2 | NTAN1/NM173474.3 | TRPVI/NM080705.3 |
| DPAGT1/NM001382.3 | NTF3/NM002527.4 | TRPV1/NM080706.3 |
| DPCD/NM015448.1 | NTM/NM001144058.1 | TRPV2/NM016113.4 |
| DPEP1/NM004413.3 | NTMT1/NM001286796.1 | TRPV3/NM145068.3 |
| DPEP2/NM022355.3 | NTMT1/NM001286797.1 | TRPV4/NM021625.4 |
| DPEP3/NM022357.3 | NTMT1/NM001286799.1 | TRPV5/NM019841.6 |
| DPF1/NM001135155.2 | NTMT1/NM014064.3 | TRPV6/NM018646.5 |
| DPF2/NM006268.4 | NTN1/NM004822.2 | TRRAP/NM003496.3 |
| DPF3/NM001280542.1 | NTN3/NM006181.2 | TRUB1/NM139169.4 |
| DPH1/NM001383.3 | NTN4/NM021229.3 | TRUB2/NM015679.1 |
| DPH2/NM001384.4 | NTN5/NM145807.2 | TSACC/NM001304818.1 |
| DPH3/NM206831.2 | NTNG1/NM001113226.2 | TSACC/NM001304819.1 |
| DPH3P1/NM080750.4 | NTNG2/NM032536.2 | TSACC/NM001304820.1 |
| DPH5/NM001077394.1 | NTPCR/NM032324.1 | TSACC/NM144627.4 |
| DPH6/NM080650.3 | NTRK1/NM002529.3 | TSC1/NM000368.4 |
| DPH7/NM138778.3 | NTS/NM006183.4 | TSC22D1/NM006022.3 |
| DPM1/NM003859.2 | NTSR1/NM002531.2 | TSC22D2/NM014779.3 |
| DPM2/NM003863.3 | NTSR2/NM012344.3 | TSC22D4/NM030935.4 |
| DPM3/NM153741.1 | NUAK1/NM014840.2 | TSEN15/NM052965.3 |
| DPP10/NM020868.4 | NUAK2/NM030952.2 | TSEN2/NM001145392.1 |
| DPP4/NM001935.3 | NUBP1/NM002484.3 | TSEN34/NM001077446.3 |
| DPP6/NM130797.3 | NUBP2/NM012225.3 | TSEN34/NM001282332.1 |
| DPP7/NM013379.2 | NUBPL/NM025152.2 | TSEN34/NM024075.4 |
| DPP8/NM001320875.1 | NUCB1/NM006184.5 | TSEN54/NM207346.2 |
| DPP8/NM197960.3 | NUCB2/NM005013.2 | TSFM/NM005726.5 |
| DPP9-AS1/NM001242901.1 | NUCKS1/NM022731.4 | TSG101/NM006292.3 |
| DPP9/NM139159.4 | NUDC/NM006600.3 | TSGA10IP/NM152762.2 |
| DPPA2/NM138815.3 | NUDCD1/NM032869.3 | TSGA13/NM001304968.1 |
| DPPA3/NM199286.3 | NUDCD2/NM145266.4 | TSGA13/NM052933.3 |
| DPPA4/NM018189.3 | NUDCD3/NM015332.3 | TSHB/NM000549.4 |
| DPPA5/NM001025290.2 | NUDT1/NM002452.3 | TSHR/NM000369.2 |
| DPRX/NM001012728.1 | NUDT1/NM198950.1 | TSHZ1/NM005786.5 |
| DPT/NM001937.4 | NUDT1/NM198953.1 | TSHZ2/NM173485.5 |
| DPY19L2/NM173812.4 | NUDT12/NM031438.3 | TSHZ3/NM020856.2 |
| DPY19L3/NM001172774.1 | NUDT13/NM015901.5 | TSKS/NM021733.1 |
| DPY19L3/NM207325.2 | NUDT14/NM177533.4 | TSKU/NM001258210.1 |
| DPY19L4/NM181787.2 | NUDT15/NM018283.3 | TSKU/NM015516.3 |
| DPY30/NM032574.3 | NUDT16/NM152395.2 | TSLP/NM033035.4 |
| DPYD/NM000110.3 | NUDT16L1/NM032349.3 | TSN/NM004622.2 |
| DPYS/NM001385.2 | NUDT17/NM001012758.2 | TSNAX/NM005999.2 |
| DPYSL2/NM001386.5 | NUDT18/NM024815.3 | TSNAXIP1/NM018430.3 |
| DPYSL3/NM001387.2 | NUDT19/NM001105570.1 | TSPAN1/NM005727.3 |
| DPYSL4/NM006426.2 | NUDT2/NM001244390.1 | TSPAN10/NM001290212.1 |
| DPYSL5/NM001253723.1 | NUDT21/NM007006.2 | TSPAN11/NM001080509.2 |
| DPYSL5/NM001253724.1 | NUDT22/NM001128612.2 | TSPAN12/NM012338.3 |
| DPYSL5/NM020134.3 | NUDT22/NM032344.3 | TSPAN13/NM014399.3 |
| DQX1/NM133637.2 | NUDT3/NM006703.3 | TSPAN14/NM030927.2 |
| DR1/NM001938.2 | NUDT4/NM199040.3 | TSPAN15/NM012339.3 |
| DRAM1/NM018370.2 | NUDT5/NM014142.3 | TSPAN16/NM012466.3 |
| DRAM2/NM178454.4 | NUDT6/NM007083.4 | TSPAN17/NM012171.2 |
| DRAP1/NM006442.3 | NUDT7/NM001105663.2 | TSPAN18/NM130783.4 |
| DRAXIN/NM198545.3 | NUDT8/NM181843.2 | TSPAN19/NM001100917.1 |
| DRC1/NM145038.4 | NUDT9/NM024047.4 | TSPAN2/NM005725.5 |
| DRC3/NM031294.3 | NUF2/NM031423.3 | TSPAN3/NM005724.5 |
| DRC7/NM032269.5 | NUFIP1/NM012345.2 | TSPAN31/NM005981.3 |
| DRD1/NM000794.3 | NUFIP2/NM020772.2 | TSPAN32/NM139022.2 |
| DRD2/NM000795.3 | NUGGC/NM001010906.1 | TSPAN33/NM178562.4 |
| DRD3/NM000796.5 | NUMA1/NM006185.3 | TSPAN4/NM001025234.1 |
| DRD3/NM001282563.2 | NUMB/NM003744.5 | TSPAN4/NM001025235.1 |
| DRD3/NM001290809.1 | NUMBL/NM004756.4 | TSPAN4/NM001025237.1 |
| DRD4/NM000797.3 | NUP107/NM020401.2 | TSPAN4/NM001025238.1 |
| DRD5/NM000798.4 | NUP133/NM018230.2 | TSPAN5/NM005723.3 |
| DRG1/NM004147.3 | NUP153/NM005124.3 | TSPAN8/NM004616.2 |
| DRG2/NM001388.4 | NUP155/NM153485.2 | TSPAN9/NM001168320.1 |
| DRGX/NM001276451.1 | NUP160/NM015231.2 | TSPEAR/NM144991.2 |
| DRICH1/NM016449.3 | NUP188/NM015354.2 | TSPO/NM000714.5 |
| DROSHA/NM013235.4 | NUP210/NM024923.3 | TSPO/NM001256530.1 |
| DSC1/NM024421.2 | NUP210L/NM207308.2 | TSPO/NM001256531.1 |
| DSC2/NM024422.4 | NUP214/NM005085.3 | TSPO2/NM001010873.2 |
| DSCAM/NM001389.3 | NUP35/NM138285.4 | TSPO2/NM001159726.1 |
| DSCAML1/NM020693.3 | NUP37/NM024057.3 | TSPOAP1/NM004758.3 |
| DSCC1/NM024094.2 | NUP43/NM198887.2 | TSPYL1/NM003309.3 |
| DSCR3/NM006052.1 | NUP50/NM007172.3 | TSPYL4/NM021648.4 |
| DSCR4/NM005867.3 | NUP54/NM017426.3 | TSPYL5/NM033512.2 |
| DSE/NM001080976.2 | NUP58/NM001008564.1 | TSPYL6/NM001003937.2 |
| DSE/NM013352.3 | NUP62/NM001193357.1 | TSR1/NM018128.4 |
| DSEL/NM032160.2 | NUP62/NM012346.4 | TSSC1/NM003310.2 |
| DSG2/NM001943.4 | NUP62/NM016553.4 | TSSC4/NM001297659.1 |
| DSG3/NM001944.2 | NUP62/NM153719.3 | TSSC4/NM005706.3 |
| DSG4/NM177986.3 | NUP88/NM002532.5 | TSSK1B/NM032028.3 |
| DSN1/NM001145315.1 | NUP93/NM014669.4 | TSSK2/NM053006.4 |
| DSN1/NM001145316.1 | NUP98/NM016320.4 | TSSK3/NM052841.3 |
| DSP/NM004415.3 | NUPL2/NM007342.2 | TSSK4/NM174944.3 |
| DSPP/NM014208.3 | NUPR1/NM012385.2 | TSSK6/NM032037.3 |
| DST/NM001144770.1 | NUPR2/NM001145712.1 | TST/NM001270483.1 |
| DSTN/NM006870.3 | NUS1/NM138459.3 | TST/NM003312.5 |
| DSTYK/NM015375.2 | NUSAP1/NM016359.4 | TSTA3/NM003313.3 |
| DTD1/NM080820.5 | NUTF2/NM001322040.1 | TSTD1/NM001113205.1 |
| DTD2/NM080664.2 | NUTF2/NM005796.2 | TSTD2/NM139246.4 |
| DTHD1/NM001170700.2 | NUTM1/NM175741.2 | TSTD3/NM001195131.1 |
| DTL/NM016448.3 | NUTM2A/NM001099338.1 | TTBK1/NM032538.1 |
| DTNA/NM001392.4 | NUTM2B/NM001278495.1 | TTC1/NM001282500.1 |
| DTNB/NM021907.4 | NUTM2F/NM017561.1 | TTC13/NM024525.4 |
| DTNBP1/NM032122.4 | NUTM2G/NM001170741.1 | TTC16/NM144965.2 |
| DTWD1/NM001144955.1 | NVL/NM002533.3 | TTC17/NM018259.5 |
| DTWD2/NM173666.3 | NWD2/NM001144990.1 | TTC19/NM017775.3 |
| DTX1/NM004416.2 | NXF1/NM006362.4 | TTC21A/NM001105513.2 |
| DTX2/NM001102595.1 | NXN/NM022463.4 | TTC21B/NM024753.4 |
| DTX3/NM001286246.1 | NXNL1/NM138454.1 | TTC22/NM017904.3 |
| DTX3L/NM138287.3 | NXNL2/NM001161625.1 | TTC23/NM001288615.1 |
| DTX4/NM015177.1 | NXPE2/NM182495.5 | TTC23/NM001288616.1 |
| DTYMK/NM012145.3 | NXPE3/NM001134456.1 | TTC23L/NM144725.3 |
| DUOX1/NM175940.2 | NXPE3/NM145037.2 | TTC24/NM001105669.2 |
| DUOX2/NM014080.4 | NXPE4/NM001077639.1 | TTC25/NM031421.3 |
| DUOXA1/NM144565.3 | NXPH1/NM152745.2 | TTC26/NM024926.3 |
| DUOXA2/NM207581.3 | NXPH2/NM007226.2 | TTC27/NM017735.4 |
| DUPD1/NM001003892.1 | NXPH3/NM007225.2 | TTC28/NM001145418.1 |
| DUS1L/NM022156.4 | NXPH4/NM007224.3 | TTC29/NM031956.3 |
| DUS2/NM001271762.1 | NXT1/NM013248.2 | TTC3/NM001001894.1 |
| DUS3L/NM020175.2 | NYAP1/NM173564.3 | TTC3/NM003316.3 |
| DUSP1/NM004417.3 | NYNRIN/NM025081.2 | TTC30A/NM152275.3 |
| DUSP10/NM007207.5 | OAF/NM178507.2 | TTC30B/NM152517.2 |
| DUSP11/NM003584.2 | OAS1/NM001032409.2 | TTC31/NM022492.4 |
| DUSP12/NM007240.2 | OAS2/NM016817.2 | TTC32/NM001008237.2 |
| DUSP14/NM007026.3 | OAS3/NM006187.3 | TTC33/NM012382.2 |
| DUSP15/NM080611.4 | OASL/NM003733.3 | TTC34/NM001242672.1 |
| DUSP16/NM030640.2 | OAT/NM001322970.1 | TTC36/NM001080441.1 |
| DUSP18/NM001304795.1 | OAZ1/NM004152.3 | TTC37/NM014639.3 |
| DUSP19/NM080876.3 | OAZ2/NM002537.3 | TTC38/NM017931.2 |
| DUSP2/NM004418.3 | OAZ3/NM016178.2 | TTC39A/NM001144832.2 |
| DUSP22/NM020185.4 | OBFC1/NM024928.4 | TTC39B/NM152574.2 |
| DUSP23/NM001319658.1 | OBP2B/NM001288987.1 | TTC39C/NM153211.3 |
| DUSP23/NM017823.4 | OBSCN/NM001271223.2 | TTC4/NM004623.4 |
| DUSP26/NM001305116.1 | OBSL1/NM015311.2 | TTC5/NM138376.2 |
| DUSP26/NM024025.2 | OC90/NM001080399.2 | TTC6/NM001310135.1 |
| DUSP27/NM001080426.1 | OCEL1/NM024578.2 | TTC7A/NM020458.3 |
| DUSP28/NM001033575.1 | OCIAD1/NM001079839.2 | TTC7B/NM001010854.1 |
| DUSP3/NM004090.3 | OCIAD1/NM017830.3 | TTC8/NM198309.3 |
| DUSP4/NM001394.6 | OCIAD2/NM001014446.2 | TTC9/NM015351.1 |
| DUSP5/NM004419.3 | OCLM/NM022375.3 | TTC9B/NM152479.5 |
| DUSP6/NM001946.3 | OCLN/NM001205254.1 | TTC9C/NM001318812.1 |
| DUSP7/NM001947.3 | OCLN/NM002538.3 | TTC9C/NM001318813.1 |
| DUSP8/NM004420.2 | OCM/NM001097622.1 | TTF1/NM007344.3 |
| DUT/NM001025248.1 | OCM2/NM006188.3 | TTF2/NM003594.3 |
| DUX4/NM001293798.1 | OCSTAMP/NM080721.2 | TTI1/NM014657.2 |
| DUXA/NM001012729.1 | ODAM/NM017855.3 | TTI2/NM025115.3 |
| DVL1/NM004421.2 | ODC1/NM001287189.1 | TTK/NM003318.4 |
| DVL2/NM004422.2 | ODC1/NM002539.2 | TTL/NM153712.4 |
| DVL3/NM004423.3 | ODF2/NM153437.2 | TTLL10/NM001130045.1 |
| DXO/NM005510.3 | ODF2L/NM020729.2 | TTLL11/NM001139442.1 |
| DYDC1/NM138812.3 | ODF3/NM053280.4 | TTLL12/NM015140.3 |
| DYDC2/NM001270041.1 | ODF3B/NM001014440.3 | TTLL2/NM031949.4 |
| DYM/NM017653.3 | ODF3L1/NM175881.3 | TTLL3/NM001025930.3 |
| DYNAP/NM173629.2 | ODF3L2/NM182577.2 | TTLL4/NM014640.4 |
| DYNC1H1/NM001376.4 | ODF4/NM153007.4 | TTLL5/NM015072.4 |
| DYNC1I1/NM004411.4 | OGDH/NM002541.3 | TTLL6/NM001130918.1 |
| DYNC1I2/NM001271785.1 | OGDHL/NM018245.2 | TTLL7/NM024686.4 |
| DYNC1I2/NM001378.2 | OGFOD1/NM018233.3 | TTN/NM133378.4 |
| DYNC1LI2/NM006141.2 | OGFOD2/NM001304833.1 | TTPA/NM000370.3 |
| DYNC2H1/NM001080463.1 | OGFOD3/NM024648.2 | TTPAL/NM001039199.2 |
| DYNC2LI1/NM016008.3 | OGFR/NM007346.2 | TTR/NM000371.3 |
| DYNLL1/NM001037494.1 | OGFRL1/NM024576.4 | TTYH1/NM020659.3 |
| DYNLL1/NM003746.2 | OGG1/NM016821.2 | TTYH2/NM032646.5 |
| DYNLL2/NM080677.2 | OGN/NM033014.3 | TTYH3/NM025250.2 |
| DYNLRB1/NM014183.3 | OIP5/NM007280.1 | TUB/NM003320.4 |
| DYNLRB2/NM130897.2 | OIT3/NM152635.2 | TUBA1A/NM001270399.1 |
| DYNLT1/NM006519.3 | OLFM1/NM001282611.1 | TUBA1B/NM006082.2 |
| DYRK1A/NM130436.2 | OLFM2/NM001304347.1 | TUBA3D/NM080386.3 |
| DYRK1B/NM004714.2 | OLFM3/NM058170.3 | TUBA3E/NM207312.2 |
| DYRK2/NM006482.2 | OLFM4/NM006418.4 | TUBA4A/NM006000.2 |
| DYRK3/NM003582.2 | OLFML1/NM198474.3 | TUBA8/NM018943.2 |
| DYRK4/NM003845.2 | OLFML2A/NM182487.3 | TUBAL3/NM024803.2 |
| DYSF/NM001130987.1 | OLFML2B/NM015441.2 | TUBB/NM178014.3 |
| DYTN/NM001093730.1 | OLFML3/NM020190.3 | TUBB1/NM030773.3 |
| DYX1C1/NM130810.3 | OLIG1/NM138983.2 | TUBB2A/NM001069.2 |
| DZANK1/NM001099407.1 | OLIG3/NM175747.2 | TUBB2B/NM178012.4 |
| DZIP1/NM198968.3 | OLR1/NM002543.3 | TUBB3/NM006086.3 |
| DZIP1L/NM173543.2 | OMA1/NM145243.4 | TUBB4A/NM001289129.1 |
| DZIP3/NM014648.3 | OMD/NM005014.2 | TUBB4A/NM006087.3 |
| E2F1/NM005225.2 | OMG/NM002544.4 | TUBB4B/NM006088.5 |
| E2F2/NM004091.3 | OMP/NM006189.1 | TUBB6/NM001303524.1 |
| E2F3/NM001949.5 | ONECUT1/NM004498.2 | TUBB6/NM032525.2 |
| E2F4/NM001950.3 | ONECUT2/NM004852.2 | TUBB8/NM177987.2 |
| E2F5/NM001951.3 | ONECUT3/NM001080488.1 | TUBD1/NM016261.3 |
| E2F6/NM198256.3 | OOEP/NM001080507.2 | TUBE1/NM016262.4 |
| E2F7/NM203394.2 | OOSP2/NM173801.4 | TUBG1/NM001070.4 |
| E2F8/NM001256371.1 | OPA3/NM025136.3 | TUBG2/NM016437.2 |
| E2F8/NM001256372.1 | OPALIN/NM033207.4 | TUBGCP2/NM006659.3 |
| E2F8/NM024680.3 | OPCML/NM002545.4 | TUBGCP3/NM006322.5 |
| E4F1/NM004424.4 | OPLAH/NM017570.4 | TUBGCP4/NM014444.4 |
| EAF1/NM033083.6 | OPN1SW/NM001708.2 | TUBGCP5/NM001102610.1 |
| EAF2/NM018456.5 | OPN3/NM014322.2 | TUBGCP6/NM020461.3 |
| EAPP/NM018453.3 | OPN4/NM033282.3 | TUFT1/NM020127.2 |
| EARS2/NM001083614.1 | OPN5/NM181744.3 | TULP2/NM003323.2 |
| EBAG9/NM001278938.1 | OPRD1/NM000911.3 | TULP3/NM003324.4 |
| EBAG9/NM004215.4 | OPRL1/NM000913.5 | TULP4/NM020245.4 |
| EBAG9/NM198120.2 | OPRL1/NM001200019.1 | TUSC1/NM001004125.2 |
| EBF1/NM024007.4 | OPRM1/NM001008504.3 | TUSC2/NM007275.2 |
| EBF3/NM001005463.2 | OPTN/NM021980.4 | TUSC3/NM006765.3 |
| EBF4/NM001110514.1 | OR10A2/NM001004460.1 | TUSC5/NM172367.2 |
| EBI3/NM005755.2 | OR10A3/NM001003745.1 | TUT1/NM022830.2 |
| EBLN1/NM001199938.1 | OR10A4/NM207186.2 | TVP23A/NM001079512.3 |
| EBLN2/NM018029.4 | OR10A5/NM178168.1 | TVP23B/NM016078.5 |
| EBNA1BP2/NM006824.2 | OR10A6/NM001004461.1 | TVP23C-CDRT4/NM001204478.1 |
| EBPL/NM032565.4 | OR10A7/NM001005280.1 | TWF1/NM002822.4 |
| ECD/NM007265.2 | OR10AD1/NM001004134.1 | TWF2/NM007284.3 |
| ECE1/NM001397.2 | OR10AG1/NM001005491.1 | TWIST2/NM057179.2 |
| ECE2/NM014693.3 | OR10C1/NM013941.3 | TWISTNB/NM001002926.1 |
| ECEL1/NM004826.3 | OR10G2/NM001005466.2 | TWSG1/NM020648.5 |
| ECH1/NM001398.2 | OR10G3/NM001005465.1 | TXK/NM003328.2 |
| ECHDC1/NM001002030.1 | OR10G4/NM001004462.1 | TXLNA/NM175852.3 |
| ECHDC2/NM001198961.1 | OR10G7/NM001004463.1 | TXLNB/NM153235.3 |
| ECHDC3/NM024693.4 | OR10G8/NM001004464.1 | TXN/NM003329.3 |
| ECHS1/NM004092.3 | OR10G9/NM001001953.1 | TXN2/NM012473.3 |
| ECI1/NM001919.3 | OR10H1/NM013940.2 | TXNDC11/NM015914.6 |
| ECI2/NM001166010.1 | OR10H2/NM013939.2 | TXNDC12/NM015913.3 |
| ECI2/NM006117.2 | OR10H3/NM013938.1 | TXNDC15/NM024715.3 |
| ECM1/NM004425.3 | OR10H4/NM001004465.1 | TXNDC16/NM020784.2 |
| ECM2/NM001393.3 | OR10H5/NM001004466.1 | TXNDC17/NM032731.3 |
| ECSCR/NM001077693.3 | OR10J3/NM001004467.1 | TXNDC2/NM032243.5 |
| ECSIT/NM016581.4 | OR10J5/NM001004469.1 | TXNDC5/NM030810.3 |
| ECT2/NM018098.5 | OR10K1/NM001004473.1 | TXNDC8/NM001003936.3 |
| ECT2L/NM001195037.2 | OR10K2/NM001004476.1 | TXNDC9/NM005783.3 |
| EDAR/NM022336.3 | OR10P1/NM206899.1 | TXNIP/NM006472.5 |
| EDARADD/NM080738.3 | OR10Q1/NM001004471.2 | TXNL1/NM004786.2 |
| EDC3/NM001142443.1 | OR10R2/NM001004472.1 | TXNL4A/NM006701.4 |
| EDC4/NM014329.4 | OR10S1/NM001004474.1 | TXNL4B/NM001142317.1 |
| EDDM3A/NM006683.4 | OR10T2/NM001004475.1 | TXNL4B/NM001142318.1 |
| EDDM3B/NM022360.4 | OR10V1/NM001005324.1 | TXNL4B/NM017853.2 |
| EDEM1/NM014674.2 | OR10W1/NM207374.3 | TXNRD1/NM182743.2 |
| EDEM2/NM018217.2 | OR10X1/NM001004477.1 | TXNRD2/NM006440.4 |
| EDEM3/NM025191.3 | OR10Z1/NM001004478.1 | TXNRD3/NM052883.1 |
| EDF1/NM003792.3 | OR11A1/NM013937.3 | TXNRD3NB/NM001039783.1 |
| EDIL3/NM005711.4 | OR11G2/NM001005503.1 | TYK2/NM003331.4 |
| EDN1/NM001955.4 | OR11H1/NM001005239.1 | TYMP/NM001113756.2 |
| EDN2/NM001956.4 | OR11H12/NM001013354.1 | TYMS/NM001071.2 |
| EDN3/NM207034.2 | OR11H2/NM001197287.1 | TYMSOS/NM001012716.2 |
| EDNRA/NM001957.3 | OR11H4/NM001004479.1 | TYRO3/NM006293.3 |
| EDNRB/NM000115.3 | OR11H6/NM001004480.1 | TYROBP/NM003332.3 |
| EDNRB/NM001122659.2 | OR11L1/NM001001959.1 | TYSND1/NM173555.3 |
| EDRF1/NM015608.2 | OR12D2/NM013936.3 | TYW1/NM018264.3 |
| EED/NM003797.4 | OR12D3/NM030959.2 | TYW1B/NM001145440.2 |
| EEF1A1/NM001402.5 | OR13A1/NM001004297.2 | TYW3/NM138467.2 |
| EEF1A2/NM001958.3 | OR13C2/NM001004481.1 | TYW5/NM001039693.2 |
| EEF1AKMT1/NM174928.2 | OR13C3/NM001001961.1 | U2AF1/NM006758.2 |
| EEF1B2/NM021121.3 | OR13C4/NM001001919.1 | U2AF1L5/NM001320646.1 |
| EEF1D/NM001130053.2 | OR13C5/NM001004482.1 | U2AF2/NM007279.2 |
| EEF1E1/NM004280.4 | OR13C8/NM001004483.1 | U2SURP/NM001080415.1 |
| EEF1G/NM001404.4 | OR13C9/NM001001956.1 | UACA/NM018003.2 |
| EEF2/NM001961.3 | OR13D1/NM001004484.1 | UAP1/NM001324116.1 |
| EEF2K/NM013302.3 | OR13F1/NM001004485.1 | UAP1L1/NM207309.2 |
| EEF2KMT/NM201400.3 | OR13G1/NM001005487.1 | UBA2/NM005499.2 |
| EEFSEC/NM021937.4 | OR13J1/NM001004487.1 | UBA3/NM003968.3 |
| EEPD1/NM030636.2 | OR14A16/NM001001966.1 | UBA5/NM024818.4 |
| EFCAB1/NM024593.3 | OR14C36/NM001001918.1 | UBA52/NM001321017.1 |
| EFCAB11/NM145231.3 | OR14I1/NM001004734.1 | UBA52/NM001321019.1 |
| EFCAB12/NM207307.2 | OR14J1/NM030946.1 | UBA52/NM003333.4 |
| EFCAB13/NM152347.4 | OR1A1/NM014565.2 | UBA6/NM018227.5 |
| EFCAB14/NM014774.2 | OR1A2/NM012352.2 | UBA7/NM003335.2 |
| EFCAB2/NM032328.3 | OR1B1/NM001004450.1 | UBAC1/NM016172.2 |
| EFCAB3/NM173503.3 | OR1C1/NM012353.2 | UBAC2/NM001144072.1 |
| EFCAB5/NM198529.3 | OR1D2/NM002548.2 | UBALD1/NM145253.2 |
| EFCAB6/NM022785.3 | OR1D5/NM014566.1 | UBALD2/NM182565.3 |
| EFCAB9/NM001171183.1 | OR1E1/NM003553.2 | UBAP1/NM016525.4 |
| EFCC1/NM024768.2 | OR1E2/NM003554.2 | UBAP1L/NM001163692.1 |
| EFEMP1/NM001039348.2 | OR1F1/NM012360.1 | UBAP2/NM018449.3 |
| EFEMP2/NM016938.4 | OR1G1/NM003555.1 | UBAP2L/NM014847.3 |
| EFHB/NM144715.3 | OR1I1/NM001004713.1 | UBASH3A/NM001001895.2 |
| EFHC1/NM018100.3 | OR1J1/NM001004451.1 | UBASH3B/NM032873.4 |
| EFHD1/NM025202.3 | OR1J2/NM054107.1 | UBB/NM001281716.1 |
| EFHD2/NM024329.5 | OR1J4/NM001004452.1 | UBB/NM001281717.1 |
| EFL1/NM001322845.1 | OR1K1/NM080859.1 | UBB/NM001281718.1 |
| EFL1/NM024580.5 | OR1L1/NM001005236.3 | UBB/NM001281719.1 |
| EFNA1/NM004428.2 | OR1L3/NM001005234.1 | UBB/NM001281720.1 |
| EFNA2/NM001405.3 | OR1L4/NM001005235.1 | UBB/NM018955.3 |
| EFNA3/NM004952.4 | OR1L6/NM001004453.2 | UBC/NM021009.6 |
| EFNA4/NM005227.2 | OR1L8/NM001004454.1 | UBD/NM006398.3 |
| EFNA5/NM001962.2 | OR1M1/NM001004456.1 | UBE2B/NM003337.3 |
| EFNB2/NM004093.3 | OR1N1/NM012363.1 | UBE2C/NM007019.3 |
| EFNB3/NM001406.3 | OR1N2/NM001004457.1 | UBE2D1/NM003338.4 |
| EFR3A/NM015137.5 | OR1Q1/NM012364.1 | UBE2D2/NM003339.2 |
| EFR3B/NM014971.1 | OR1S1/NM001004458.1 | UBE2D3/NM181892.3 |
| EFS/NM005864.3 | OR1S2/NM001004459.1 | UBE2D4/NM015983.3 |
| EFTUD2/NM001258353.1 | OR2A1/NM001005287.1 | UBE2E1/NM003341.4 |
| EFTUD2/NM004247.3 | OR2A12/NM001004135.1 | UBE2E2/NM152653.3 |
| EGF/NM001963.5 | OR2A14/NM001001659.1 | UBE2E3/NM001278554.1 |
| EGFL7/NM016215.4 | OR2A2/NM001005480.2 | UBE2E3/NM001278555.1 |
| EGFL7/NM201446.2 | OR2A25/NM001004488.1 | UBE2E3/NM006357.3 |
| EGFL8/NM030652.3 | OR2A4/NM030908.2 | UBE2F/NM001278305.1 |
| EGFLAM/NM152403.3 | OR2A42/NM001001802.2 | UBE2F/NM080678.2 |
| EGFR/NM005228.3 | OR2A5/NM012365.1 | UBE2G1/NM003342.4 |
| EGLN1/NM022051.2 | OR2A7/NM001005328.1 | UBE2G2/NM003343.5 |
| EGLN2/NM053046.3 | OR2AE1/NM001005276.1 | UBE2H/NM003344.3 |
| EGLN2/NM080732.3 | OR2AG1/NM001004489.2 | UBE2I/NM003345.4 |
| EGLN3/NM022073.3 | OR2AG2/NM001004490.1 | UBE2I/NM194261.2 |
| EGR1/NM001964.2 | OR2AK2/NM001004491.1 | UBE2J1/NM016021.2 |
| EGR2/NM000399.4 | OR2AP1/NM001258285.1 | UBE2J2/NM058167.2 |
| EGR2/NM001136178.1 | OR2AT4/NM001005285.1 | UBE2K/NM005339.4 |
| EGR3/NM004430.2 | OR2B11/NM001004492.1 | UBE2L3/NM003347.3 |
| EGR4/NM001965.3 | OR2B2/NM033057.2 | UBE2L6/NM004223.4 |
| EHBP1/NM015252.3 | OR2B3/NM001005226.2 | UBE2M/NM003969.3 |
| EHBP1L1/NM001099409.1 | OR2B6/NM012367.1 | UBE2N/NM003348.3 |
| EHD1/NM001282444.1 | OR2C1/NM012368.2 | UBE2O/NM022066.3 |
| EHD1/NM006795.3 | OR2C3/NM198074.4 | UBE2Q1/NM017582.6 |
| EHD2/NM014601.3 | OR2D2/NM003700.1 | UBE2Q2/NM173469.3 |
| EHD4/NM139265.3 | OR2D3/NM001004684.1 | UBE2Q2L/NM001243531.1 |
| EHF/NM012153.5 | OR2F1/NM012369.2 | UBE2QL1/NM001145161.2 |
| EHHADH/NM001966.3 | OR2F2/NM001004685.1 | UBE2R2/NM017811.3 |
| EHMT1/NM024757.4 | OR2G2/NM001001915.1 | UBE2S/NM014501.2 |
| EHMT2/NM006709.4 | OR2G3/NM001001914.1 | UBE2T/NM014176.3 |
| EI24/NM004879.4 | OR2G6/NM001013355.1 | UBE2U/NM152489.1 |
| EID1/NM014335.2 | OR2H1/NM001318022.1 | UBE2V1/NM001032288.2 |
| EID2/NM153232.3 | OR2H2/NM007160.3 | UBE2V2/NM003350.2 |
| EID2B/NM152361.2 | OR2J2/NM030905.2 | UBE2W/NM001001481.2 |
| EID3/NM001008394.2 | OR2J3/NM001005216.3 | UBE2Z/NM023079.4 |
| EIF1/NM005801.3 | OR2K2/NM205859.1 | UBE3A/NM130838.1 |
| EIF1AD/NM001242481.1 | OR2L13/NM001304535.1 | UBE3B/NM183415.2 |
| EIF1B/NM005875.2 | OR2L2/NM001004686.2 | UBE3C/NM014671.2 |
| EIF2A/NM032025.4 | OR2L3/NM001004687.1 | UBE4B/NM006048.4 |
| EIF2AK1/NM014413.3 | OR2L5/NM001258284.1 | UBFD1/NM019116.2 |
| EIF2AK2/NM002759.3 | OR2L8/NM001001963.1 | UBIAD1/NM013319.2 |
| EIF2AK3/NM004836.6 | OR2M2/NM001004688.1 | UBL3/NM007106.3 |
| EIF2AK4/NM001013703.3 | OR2M3/NM001004689.1 | UBL4B/NM203412.1 |
| EIF2B1/NM001414.3 | OR2M4/NM017504.1 | UBL5/NM001048241.2 |
| EIF2B3/NM020365.4 | OR2M5/NM001004690.1 | UBL5/NM024292.3 |
| EIF2B4/NM001034116.1 | OR2M7/NM001004691.1 | UBL7/NM032907.4 |
| EIF2B5/NM003907.2 | OR2S2/NM019897.2 | UBLCP1/NM145049.3 |
| EIF2D/NM006893.2 | OR2T1/NM030904.1 | UBN1/NM001079514.2 |
| EIF2S1/NM004094.4 | OR2T10/NM001004693.1 | UBN2/NM173569.3 |
| EIF2S2/NM003908.4 | OR2T11/NM001001964.1 | UBOX5/NM014948.3 |
| EIF3A/NM003750.2 | OR2T12/NM001004692.1 | UBP1/NM001128161.1 |
| EIF3B/NM003751.3 | OR2T2/NM001004136.1 | UBP1/NM014517.4 |
| EIF3C/NM001199142.1 | OR2T27/NM001001824.1 | UBQLN1/NM013438.4 |
| EIF3C/NM003752.4 | OR2T29/NM001004694.2 | UBQLN3/NM017481.2 |
| EIF3CL/NM001317856.1 | OR2T3/NM001005495.1 | UBQLN4/NM020131.4 |
| EIF3D/NM003753.3 | OR2T33/NM001004695.1 | UBQLNL/NM145053.4 |
| EIF3E/NM001568.2 | OR2T34/NM001001821.1 | UBR1/NM174916.2 |
| EIF3F/NM003754.2 | OR2T35/NM001001827.1 | UBR2/NM015255.2 |
| EIF3H/NM003756.2 | OR2T4/NM001004696.1 | UBR3/NM172070.3 |
| EIF3I/NM003757.3 | OR2T5/NM001004697.1 | UBR4/NM020765.2 |
| EIF3J/NM003758.3 | OR2T6/NM001005471.1 | UBR5/NM015902.5 |
| EIF3L/NM016091.3 | OR2T8/NM001005522.1 | UBR7/NM175748.3 |
| EIF3M/NM006360.5 | OR2V1/NM001258283.1 | UBTD1/NM024954.4 |
| EIF4A1/NM001416.3 | OR2V2/NM206880.1 | UBTD2/NM152277.2 |
| EIF4A3/NM014740.3 | OR2W1/NM030903.3 | UBTF/NM014233.3 |
| EIF4B/NM001417.5 | OR2W3/NM001001957.2 | UBTFL1/NM001143975.1 |
| EIF4E/NM001968.3 | OR2W5/NM001004698.2 | UBXN1/NM001286077.1 |
| EIF4E2/NM004846.3 | OR2Y1/NM001001657.1 | UBXN10/NM152376.4 |
| EIF4E3/NM001134651.1 | OR2Z1/NM001004699.2 | UBXN11/NM183008.2 |
| EIF4EBP1/NM004095.3 | OR3A1/NM002550.2 | UBXN2A/NM181713.3 |
| EIF4EBP2/NM004096.4 | OR3A2/NM002551.3 | UBXN2B/NM001077619.1 |
| EIF4EBP3/NM003732.2 | OR3A3/NM012373.2 | UBXN4/NM014607.3 |
| EIF4G1/NM198241.2 | OR4A15/NM001005275.1 | UBXN6/NM025241.2 |
| EIF4G2/NM001172705.1 | OR4A16/NM001005274.1 | UBXN7/NM015562.1 |
| EIF4G2/NM001418.3 | OR4A47/NM001005512.2 | UBXN8/NM005671.3 |
| EIF4G3/NM003760.4 | OR4A5/NM001005272.3 | UCHL1/NM004181.4 |
| EIF4H/NM022170.1 | OR4B1/NM001005470.1 | UCHL3/NM006002.4 |
| EIF5/NM001969.4 | OR4C11/NM001004700.2 | UCHL5/NM015984.3 |
| EIF5/NM183004.4 | OR4C12/NM001005270.4 | UCK1/NM031432.3 |
| EIF5A/NM001143761.1 | OR4C13/NM001001955.2 | UCK2/NM012474.4 |
| EIF5A/NM001143762.1 | OR4C15/NM001001920.1 | UCKL1/NM001193379.1 |
| EIF5A/NM001970.4 | OR4C16/NM001004701.2 | UCMA/NM145314.2 |
| EIF5A2/NM020390.5 | OR4C3/NM001004702.1 | UCN/NM003353.3 |
| EIF5AL1/NM001099692.1 | OR4C45/NM001005513.1 | UCN2/NM033199.3 |
| EIF5B/NM015904.3 | OR4C46/NM001004703.1 | UCN3/NM053049.2 |
| EIF6/NM002212.3 | OR4C6/NM001004704.1 | UCP1/NM021833.4 |
| EIF6/NM181468.2 | OR4D1/NM012374.1 | UCP3/NM003356.3 |
| ELAC1/NM018696.2 | OR4D10/NM001004705.1 | UEVLD/NM001040697.2 |
| ELAC2/NM018127.6 | OR4D11/NM001004706.1 | UFC1/NM016406.3 |
| ELANE/NM001972.3 | OR4D2/NM001004707.3 | UFD1L/NM005659.6 |
| ELAVL1/NM001419.2 | OR4D5/NM001001965.1 | UFL1/NM015323.4 |
| ELAVL2/NM004432.3 | OR4D6/NM001004708.1 | UFSP1/NM001015072.3 |
| ELAVL4/NM021952.4 | OR4D9/NM001004711.1 | UFSP2/NM018359.3 |
| ELF1/NM172373.3 | OR4E1/NM001317107.1 | UGCG/NM003358.2 |
| ELF2/NM201999.2 | OR4E2/NM001001912.2 | UGDH/NM003359.3 |
| ELF3/NM004433.4 | OR4F15/NM001001674.1 | UGGT1/NM020120.3 |
| ELF5/NM001422.3 | OR4F16/NM001005277.1 | UGGT2/NM020121.3 |
| ELFN1/NM001128636.2 | OR4F17/NM001005240.1 | UGP2/NM006759.3 |
| ELFN2/NM052906.4 | OR4F21/NM001005504.1 | UGT1A1/NM000463.2 |
| ELK3/NM005230.3 | OR4F29/NM001005221.2 | UGT1A10/NM019075.2 |
| ELK4/NM001973.3 | OR4F3/NM001005224.1 | UGT1A3/NM019093.2 |
| ELL/NM006532.3 | OR4F4/NM001004195.2 | UGT1A4/NM007120.2 |
| ELL2/NM012081.5 | OR4F5/NM001005484.1 | UGT1A5/NM019078.1 |
| ELL3/NM025165.2 | OR4F6/NM001005326.1 | UGT1A6/NM001072.3 |
| ELMO1/NM001206480.2 | OR4K1/NM001004063.2 | UGT1A7/NM019077.2 |
| ELMO1/NM001206482.1 | OR4K13/NM001004714.1 | UGT1A8/NM019076.4 |
| ELMO1/NM014800.10 | OR4K14/NM001004712.1 | UGT1A9/NM021027.2 |
| ELMO2/NM133171.4 | OR4K15/NM001005486.1 | UGT2A1/NM006798.4 |
| ELMO3/NM024712.3 | OR4K17/NM001004715.1 | UGT2A2/NM001105677.2 |
| ELMOD2/NM153702.3 | OR4K2/NM001005501.1 | UGT2A3/NM024743.3 |
| ELMOD3/NM001135023.1 | OR4K5/NM001005483.1 | UGT2B10/NM001075.5 |
| ELN/NM000501.3 | OR4L1/NM001004717.1 | UGT2B11/NM001073.1 |
| ELOF1/NM032377.3 | OR4M1/NM001005500.1 | UGT2B15/NM001076.3 |
| ELOVL1/NM001256399.1 | OR4M2/NM001004719.2 | UGT2B17/NM001077.3 |
| ELOVL1/NM022821.3 | OR4N2/NM001004723.2 | UGT2B28/NM053039.1 |
| ELOVL2/NM017770.3 | OR4N4/NM001005241.3 | UGT2B7/NM001074.2 |
| ELOVL3/NM152310.2 | OR4N5/NM001004724.1 | UGT3A1/NM152404.3 |
| ELOVL4/NM022726.3 | OR4P4/NM001004124.2 | UGT3A2/NM174914.3 |
| ELOVL5/NM001301856.1 | OR4Q3/NM172194.1 | UGT8/NM001128174.2 |
| ELOVL5/NM021814.4 | OR4S1/NM001004725.1 | UGT8/NM001322114.1 |
| ELOVL6/NM001130721.1 | OR4S2/NM001004059.2 | UGT8/NM003360.4 |
| ELOVL6/NM024090.2 | OR4X1/NM001004726.1 | UHMK1/NM175866.4 |
| ELOVL7/NM024930.2 | OR4X2/NM001004727.1 | UHRF1/NM013282.4 |
| ELP2/NM018255.2 | OR51A2/NM001004748.1 | UHRF1BP1/NM017754.3 |
| ELP3/NM018091.5 | OR51A4/NM001005329.1 | UHRF1BP1L/NM015054.1 |
| ELP4/NM019040.4 | OR51A7/NM001004749.1 | UHRF2/NM152896.2 |
| ELP5/NM015362.4 | OR51B2/NM033180.4 | UIMC1/NM001199297.2 |
| ELP5/NM203415.1 | OR51B4/NM033179.2 | UIMC1/NM016290.4 |
| EMC1/NM015047.2 | OR51B5/NM001005567.2 | ULBP1/NM025218.3 |
| EMC10/NM206538.3 | OR51B6/NM001004750.1 | ULBP2/NM025217.3 |
| EMC2/NM014673.3 | OR51D1/NM001004751.2 | ULBP3/NM024518.2 |
| EMC3/NM018447.3 | OR51E1/NM152430.3 | ULK1/NM003565.2 |
| EMC4/NM016454.3 | OR51F1/NM001004752.1 | ULK2/NM014683.3 |
| EMC6/NM031298.3 | OR51F2/NM001004753.1 | ULK3/NM001284364.2 |
| EMC7/NM020154.2 | OR51G1/NM001005237.1 | ULK4/NM017886.3 |
| EMC8/NM006067.4 | OR51G2/NM001005238.1 | UMAD1/NM001302349.1 |
| EMC9/NM016049.3 | OR51I1/NM001005288.2 | UMOD/NM003361.3 |
| EMCN/NM016242.3 | OR51I2/NM001004754.2 | UMODL1/NM173568.3 |
| EME1/NM152463.2 | OR51L1/NM001004755.1 | UMPS/NM000373.3 |
| EME2/NM001257370.1 | OR51M1/NM001004756.2 | UNC119B/NM001080533.2 |
| EMG1/NM006331.7 | OR51Q1/NM001004757.2 | UNC13A/NM001080421.2 |
| EMID1/NM133455.3 | OR51S1/NM001004758.1 | UNC13B/NM006377.3 |
| EMILIN1/NM007046.3 | OR51T1/NM001004759.1 | UNC13D/NM199242.2 |
| EMILIN2/NM032048.2 | OR51V1/NM001004760.2 | UNC45A/NM018671.4 |
| EMILIN3/NM052846.1 | OR52A1/NM012375.2 | UNC45B/NM173167.3 |
| EML1/NM001008707.1 | OR52A5/NM001005160.2 | UNC50/NM014044.5 |
| EML2/NM012155.2 | OR52B2/NM001004052.1 | UNC5A/NM133369.2 |
| EML3/NM153265.2 | OR52B4/NM001005161.3 | UNC5B/NM170744.4 |
| EML4/NM019063.4 | OR52B6/NM001005162.2 | UNC5C/NM003728.3 |
| EML6/NM001039753.2 | OR52D1/NM001005163.2 | UNC5CL/NM173561.2 |
| EMP1/NM001423.2 | OR52E2/NM001005164.2 | UNC93A/NM018974.3 |
| EMP2/NM001424.5 | OR52E4/NM001005165.1 | UNC93B1/NM030930.3 |
| EMP3/NM001313905.1 | OR52E6/NM001005167.1 | UNCX/NM001080461.1 |
| EMP3/NM001425.2 | OR52E8/NM001005168.1 | UNG/NM080911.2 |
| EMSY/NM020193.4 | OR52H1/NM001005289.1 | UNK/NM001080419.2 |
| EMX1/NM004097.2 | OR52I1/NM001005169.1 | UNKL/NM001193388.3 |
| EMX2/NM004098.3 | OR52I2/NM001005170.2 | UPB1/NM016327.2 |
| EN1/NM001426.3 | OR52J3/NM001001916.2 | UPF1/NM002911.3 |
| EN2/NM001427.3 | OR52K1/NM001005171.2 | UPF2/NM015542.3 |
| ENAH/NM001008493.1 | OR52K2/NM001005172.2 | UPF2/NM080599.2 |
| ENAM/NM031889.2 | OR52L1/NM001005173.3 | UPF3A/NM023011.3 |
| ENC1/NM001256574.1 | OR52M1/NM001004137.1 | UPK1A/NM007000.3 |
| ENDOD1/NM015036.2 | OR52N1/NM001001913.1 | UPK1B/NM006952.3 |
| ENDOG/NM004435.2 | OR52N2/NM001005174.1 | UPK2/NM006760.3 |
| ENDOU/NM001172439.1 | OR52N4/NM001005175.3 | UPK3A/NM006953.3 |
| ENDOV/NM173627.4 | OR52N5/NM001001922.2 | UPK3BL/NM001114403.2 |
| ENG/NM000118.3 | OR52R1/NM001005177.3 | UPP1/NM001287426.1 |
| ENGASE/NM001042573.2 | OR52W1/NM001005178.1 | UPP1/NM003364.3 |
| ENHO/NM198573.2 | OR56A1/NM001001917.2 | UPP2/NM173355.3 |
| ENKD1/NM032140.1 | OR56A3/NM001003443.2 | UQCC1/NM018244.4 |
| ENKUR/NM145010.3 | OR56A4/NM001005179.2 | UQCC2/NM032340.3 |
| ENO1/NM001428.3 | OR56A5/NM001146033.1 | UQCC3/NM001085372.2 |
| ENO2/NM001975.2 | OR56B1/NM001005180.2 | UQCR10/NM013387.3 |
| ENO4/NM001242699.1 | OR56B4/NM001005181.2 | UQCRB/NM006294.4 |
| ENOPH1/NM021204.4 | OR5A1/NM001004728.1 | UQCRC1/NM003365.2 |
| ENOSF1/NM001318759.1 | OR5A2/NM001001954.1 | UQCRC2/NM003366.3 |
| ENOSF1/NM017512.5 | OR5AC2/NM054106.1 | UQCRFS1/NM006003.2 |
| ENOX1/NM001127615.1 | OR5AK2/NM001005323.1 | UQCRH/NM006004.3 |
| ENOX1/NM017993.3 | OR5AN1/NM001004729.1 | UQCRHL/NM001089591.1 |
| ENPEP/NM001977.3 | OR5AP2/NM001002925.1 | URAD/NM001105577.1 |
| ENPP1/NM006208.2 | OR5AR1/NM001004730.1 | URB1/NM014825.2 |
| ENPP2/NM006209.4 | OR5AS1/NM001001921.1 | URB2/NM014777.3 |
| ENPP3/NM005021.4 | OR5AU1/NM001004731.1 | URGCP-MRPS24/NM001204871.1 |
| ENPP4/NM014936.4 | OR5B12/NM001004733.2 | URGCP/NM001290075.1 |
| ENPP5/NM001290072.1 | OR5B17/NM001005489.1 | URGCP/NM001290076.1 |
| ENPP6/NM153343.3 | OR5B2/NM001005566.2 | URI1/NM003796.3 |
| ENPP7/NM178543.4 | OR5B21/NM001005218.1 | URM1/NM030914.3 |
| ENSA/NM004436.2 | OR5B3/NM001005469.1 | UROC1/NM144639.2 |
| ENTHD1/NM152512.3 | OR5C1/NM001001923.1 | UROS/NM000375.2 |
| ENTHD2/NM144679.2 | OR5D13/NM001001967.1 | USB1/NM024598.3 |
| ENTPD1/NM001776.5 | OR5D14/NM001004735.1 | USE1/NM018467.3 |
| ENTPD2/NM203468.2 | OR5D16/NM001005496.1 | USF1/NM001276373.1 |
| ENTPD3/NM001248.3 | OR5D18/NM001001952.1 | USF1/NM007122.4 |
| ENTPD3/NM001291960.1 | OR5F1/NM003697.1 | USF2/NM003367.3 |
| ENTPD4/NM004901.4 | OR5H1/NM001005338.1 | USF3/NM001009899.3 |
| ENTPD5/NM001321988.1 | OR5H14/NM001005514.1 | USH1C/NM005709.3 |
| ENTPD6/NM001247.4 | OR5H15/NM001005515.1 | USH1G/NM173477.4 |
| ENTPD7/NM020354.3 | OR5H2/NM001005482.1 | USH2A/NM206933.2 |
| ENTPD8/NM198585.2 | OR5H6/NM001005479.1 | USHBP1/NM031941.3 |
| ENY2/NM020189.5 | OR5I1/NM006637.1 | USMG5/NM001206427.1 |
| EOGT/NM173654.2 | OR5J2/NM001005492.1 | USO1/NM003715.3 |
| EOMES/NM005442.3 | OR5K1/NM001004736.3 | USP1/NM001017416.1 |
| EP300/NM001429.3 | OR5K2/NM001004737.1 | USP1/NM003368.4 |
| EP400/NM015409.4 | OR5K3/NM001005516.1 | USP10/NM005153.2 |
| EPAS1/NM001430.4 | OR5K4/NM001005517.1 | USP12/NM182488.3 |
| EPB41/NM203342.2 | OR5L1/NM001004738.1 | USP13/NM003940.2 |
| EPB41L2/NM001431.3 | OR5L2/NM001004739.1 | USP14/NM005151.3 |
| EPB41L3/NM012307.3 | OR5M1/NM001004740.1 | USP15/NM006313.2 |
| EPB41L4A/NM022140.3 | OR5M10/NM001004741.1 | USP16/NM006447.2 |
| EPB41L4B/NM019114.4 | OR5M11/NM001005245.1 | USP17L1/NM001256873.1 |
| EPB41L5/NM001184938.2 | OR5M3/NM001004742.2 | USP17L10/NM001256852.1 |
| EPB41L5/NM001184939.1 | OR5M8/NM001005282.1 | USP17L11/NM001256854.1 |
| EPB42/NM000119.2 | OR5M9/NM001004743.1 | USP17L12/NM001256853.1 |
| EPC1/NM001272004.1 | OR5P2/NM153444.1 | USP17L13/NM001256855.1 |
| EPC2/NM015630.3 | OR5P3/NM153445.1 | USP17L15/NM001256894.1 |
| EPCAM/NM002354.2 | OR5R1/NM001004744.1 | USP17L17/NM001256857.1 |
| EPDR1/NM017549.4 | OR5T1/NM001004745.1 | USP17L18/NM001256859.1 |
| EPG5/NM020964.2 | OR5T2/NM001004746.1 | USP17L19/NM001256860.1 |
| EPGN/NM001270989.1 | OR5T3/NM001004747.1 | USP17L2/NM201402.2 |
| EPHA1/NM005232.4 | OR5V1/NM030876.5 | USP17L20/NM001256861.1 |
| EPHA10/NM001099439.1 | OR5W2/NM001001960.1 | USP17L21/NM001256862.1 |
| EPHA2/NM004431.3 | OR6A2/NM003696.2 | USP17L22/NM001256863.1 |
| EPHA3/NM005233.5 | OR6B1/NM001005281.1 | USP17L24/NM001242327.1 |
| EPHA4/NM001304536.1 | OR6B2/NM001005853.1 | USP17L25/NM001242326.1 |
| EPHA4/NM004438.4 | OR6B3/NM173351.1 | USP17L26/NM001242328.1 |
| EPHA5/NM004439.7 | OR6C1/NM001005182.1 | USP17L27/NM001242330.1 |
| EPHA6/NM001080448.2 | OR6C2/NM054105.1 | USP17L28/NM001242331.1 |
| EPHA7/NM004440.3 | OR6C3/NM054104.1 | USP17L29/NM001242332.1 |
| EPHA8/NM020526.3 | OR6C4/NM001005494.1 | USP17L3/NM001256871.1 |
| EPHB1/NM004441.4 | OR6C6/NM001005493.1 | USP17L30/NM001256867.1 |
| EPHB2/NM004442.7 | OR6C65/NM001005518.1 | USP17L4/NM001256874.1 |
| EPHB3/NM004443.3 | OR6C68/NM001005519.2 | USP17L5/NM001242329.1 |
| EPHB4/NM004444.4 | OR6C70/NM001005499.1 | USP17L7/NM001256869.1 |
| EPHB6/NM004445.5 | OR6C74/NM001005490.1 | USP17L8/NM001256872.1 |
| EPHX1/NM000120.3 | OR6C75/NM001005497.1 | USP18/NM017414.3 |
| EPHX1/NM001136018.3 | OR6C76/NM001005183.1 | USP19/NM006677.2 |
| EPHX1/NM001291163.1 | OR6F1/NM001005286.1 | USP2/NM004205.4 |
| EPHX2/NM001979.5 | OR6K2/NM001005279.1 | USP20/NM001110303.3 |
| EPHX3/NM001142886.1 | OR6K3/NM001005327.2 | USP21/NM012475.4 |
| EPHX3/NM024794.2 | OR6K6/NM001005184.1 | USP22/NM015276.1 |
| EPHX4/NM173567.4 | OR6M1/NM001005325.1 | USP24/NM015306.2 |
| EPM2A/NM005670.3 | OR6N1/NM001005185.1 | USP25/NM013396.4 |
| EPM2AIP1/NM014805.3 | OR6N2/NM001005278.1 | USP28/NM020886.3 |
| EPN1/NM001130072.1 | OR6P1/NM001160325.1 | USP29/NM020903.2 |
| EPN2/NM014964.4 | OR6Q1/NM001005186.2 | USP3/NM006537.3 |
| EPN3/NM017957.2 | OR6S1/NM001001968.1 | USP30/NM032663.4 |
| EPO/NM000799.2 | OR6T1/NM001005187.1 | USP31/NM020718.3 |
| EPOR/NM000121.3 | OR6V1/NM001001667.1 | USP32/NM032582.3 |
| EPPK1/NM031308.3 | OR6X1/NM001005188.1 | USP33/NM201624.2 |
| EPRS/NM004446.2 | OR6Y1/NM001005189.1 | USP34/NM014709.3 |
| EPS15/NM001981.2 | OR7A10/NM001005190.1 | USP35/NM020798.2 |
| EPS15L1/NM001258374.1 | OR7A17/NM030901.1 | USP36/NM001321291.1 |
| EPS8/NM004447.5 | OR7A5/NM017506.1 | USP37/NM020935.2 |
| EPS8L1/NM133180.2 | OR7C1/NM198944.1 | USP38/NM032557.6 |
| EPS8L2/NM022772.3 | OR7C2/NM012377.1 | USP39/NM001256725.1 |
| EPS8L3/NM139053.2 | OR7D2/NM175883.2 | USP4/NM003363.3 |
| EPSTI1/NM033255.3 | OR7D4/NM001005191.2 | USP40/NM018218.2 |
| EPT1/NM033505.2 | OR7E24/NM001079935.1 | USP42/NM032172.2 |
| EPX/NM000502.5 | OR7G1/NM001005192.2 | USP43/NM153210.4 |
| EPYC/NM004950.4 | OR7G2/NM001005193.1 | USP44/NM001042403.2 |
| EQTN/NM020641.2 | OR7G3/NM001001958.1 | USP44/NM001278393.1 |
| ERAL1/NM005702.3 | OR8A1/NM001005194.1 | USP44/NM032147.4 |
| ERAP1/NM016442.3 | OR8B12/NM001005195.1 | USP46/NM022832.3 |
| ERAP2/NM001130140.1 | OR8B2/NM001005468.1 | USP47/NM001282659.1 |
| ERBB2/NM004448.3 | OR8B3/NM001005467.1 | USP48/NM032236.5 |
| ERBB3/NM001982.3 | OR8B4/NM001005196.1 | USP49/NM001286554.1 |
| ERBB4/NM005235.2 | OR8B8/NM012378.1 | USP5/NM003481.2 |
| ERBIN/NM018695.3 | OR8D1/NM001002917.1 | USP50/NM203494.4 |
| ERC1/NM178039.3 | OR8D2/NM001002918.1 | USP53/NM019050.2 |
| ERC2/NM015576.2 | OR8D4/NM001005197.1 | USP54/NM152586.3 |
| ERCC1/NM001983.3 | OR8G1/NM001002905.1 | USP6/NM001304284.1 |
| ERCC2/NM000400.3 | OR8G2/NM001291438.1 | USP6/NM004505.3 |
| ERCC3/NM000122.1 | OR8H1/NM001005199.1 | USP6NL/NM001080491.3 |
| ERCC4/NM005236.2 | OR8H2/NM001005200.1 | USP7/NM003470.2 |
| ERCC5/NM000123.3 | OR8H3/NM001005201.1 | USP8/NM005154.4 |
| ERCC6/NM000124.3 | OR8I2/NM001003750.1 | USPL1/NM005800.4 |
| ERCC6L2/NM001010895.2 | OR8J1/NM001005205.2 | UST/NM005715.2 |
| EREG/NM001432.2 | OR8J3/NM001004064.1 | UTF1/NM003577.2 |
| ERF/NM006494.3 | OR8K1/NM001002907.1 | UTP11/NM016037.3 |
| ERG/NM182918.3 | OR8K3/NM001005202.1 | UTP14C/NM021645.5 |
| ERGIC1/NM001031711.2 | OR8K5/NM001004058.2 | UTP15/NM032175.3 |
| ERGIC2/NM016570.2 | OR8S1/NM001005203.2 | UTP18/NM016001.2 |
| ERGIC3/NM198398.1 | OR8U1/NM001005204.1 | UTP20/NM014503.2 |
| ERH/NM004450.2 | OR9A2/NM001001658.1 | UTP23/NM032334.2 |
| ERI1/NM153332.3 | OR9A4/NM001001656.1 | UTP3/NM020368.2 |
| ERI2/NM001142725.1 | OR9G1/NM001005213.1 | UTP4/NM032830.2 |
| ERI3/NM024066.2 | OR9G4/NM001005284.1 | UTP6/NM018428.2 |
| ERICH1/NM207332.2 | OR9I1/NM001005211.1 | UTRN/NM007124.2 |
| ERICH2/NM001289947.1 | OR9K2/NM001005243.1 | UTS2/NM021995.2 |
| ERICH3/NM001002912.4 | OR9Q1/NM001005212.3 | UTS2B/NM198152.3 |
| ERICH4/NM001130514.1 | OR9Q2/NM001005283.2 | UTS2R/NM018949.2 |
| ERICH5/NM173549.2 | ORAI1/NM032790.3 | UVRAG/NM003369.3 |
| ERICH6/NM152394.4 | ORAI2/NM032831.3 | UVSSA/NM020894.3 |
| ERICH6B/NM182542.2 | ORAI3/NM152288.2 | UXS1/NM025076.4 |
| ERLEC1/NM015701.4 | ORAOV1/NM153451.2 | VAC14/NM018052.3 |
| ERLIN1/NM001100626.1 | ORC2/NM006190.4 | VAMP1/NM014231.4 |
| ERLIN1/NM006459.3 | ORC3/NM181837.2 | VAMP2/NM014232.2 |
| ERLIN2/NM007175.6 | ORC5/NM002553.3 | VAMP3/NM004781.3 |
| ERMAP/NM001017922.1 | ORC6/NM014321.3 | VAMP4/NM003762.4 |
| ERMAP/NM018538.3 | ORM1/NM000607.2 | VAMP5/NM006634.2 |
| ERMARD/NM018341.2 | ORM2/NM000608.2 | VAMP8/NM003761.4 |
| ERMN/NM001304345.1 | ORMDL1/NM016467.4 | VANGL1/NM001172412.1 |
| ERMN/NM020711.2 | ORMDL2/NM014182.4 | VANGL1/NM138959.2 |
| ERN1/NM001433.3 | ORMDL3/NM001320801.1 | VANGL2/NM020335.2 |
| ERN2/NM033266.3 | ORMDL3/NM139280.3 | VAPA/NM003574.5 |
| ERO1A/NM014584.2 | OS9/NM006812.3 | VARS2/NM020442.5 |
| ERO1B/NM019891.3 | OSBP/NM002556.2 | VASH1/NM014909.4 |
| ERP27/NM152321.3 | OSBP2/NM030758.3 | VASH2/NM001301056.1 |
| ERP29/NM006817.3 | OSBPL10/NM017784.4 | VASN/NM138440.2 |
| ERP44/NM015051.2 | OSBPL11/NM022776.4 | VASP/NM003370.3 |
| ERRFI1/NM018948.3 | OSBPL1A/NM080597.3 | VAT1/NM006373.3 |
| ERV3-1/NM001007253.3 | OSBPL2/NM144498.2 | VAT1L/NM020927.2 |
| ERVFRD-1/NM207582.2 | OSBPL3/NM015550.3 | VAV1/NM005428.3 |
| ERVH48-1/NM001308491.1 | OSBPL5/NM020896.3 | VAV2/NM003371.3 |
| ERVMER34-1/NM024534.5 | OSBPL6/NM032523.3 | VAV3/NM006113.4 |
| ERVV-1/NM152473.2 | OSBPL7/NM145798.2 | VAX1/NM001112704.1 |
| ERVV-2/NM001191055.1 | OSBPL8/NM020841.4 | VAX2/NM012476.2 |
| ERVW-1/NM001130925.1 | OSBPL9/NM024586.5 | VCAM1/NM001078.3 |
| ERVW-1/NM014590.3 | OSCP1/NM145047.4 | VCAN/NM004385.4 |
| ESAM/NM138961.2 | OSER1/NM016470.7 | VCL/NM014000.2 |
| ESCO2/NM001017420.2 | OSGEP/NM017807.3 | VCP/NM007126.3 |
| ESD/NM001984.1 | OSGEPL1/NM022353.2 | VCPIP1/NM025054.4 |
| ESF1/NM001276380.1 | OSGIN1/NM182981.2 | VCPKMT/NM024558.2 |
| ESF1/NM016649.3 | OSGIN2/NM004337.2 | VDAC1/NM003374.2 |
| ESPL1/NM012291.4 | OSM/NM020530.5 | VDAC2/NM001184823.1 |
| ESPN/NM031475.2 | OSMR/NM003999.2 | VDAC3/NM005662.6 |
| ESPNL/NM194312.3 | OSR1/NM145260.2 | VDR/NM001017535.1 |
| ESR1/NM000125.3 | OSR2/NM001142462.2 | VEGFA/NM001171624.1 |
| ESR1/NM001122740.1 | OST4/NM001134693.1 | VEGFB/NM003377.4 |
| ESR1/NM001122741.1 | OSTC/NM021227.3 | VEGFC/NM005429.4 |
| ESR1/NM001122742.1 | OSTF1/NM012383.4 | VENTX/NM014468.3 |
| ESR2/NM001437.2 | OSTM1/NM014028.3 | VEPH1/NM001167912.1 |
| ESRP1/NM017697.3 | OSTN/NM198184.1 | VEPH1/NM024621.2 |
| ESRP2/NM024939.2 | OTOA/NM144672.3 | VEZF1/NM007146.2 |
| ESRRA/NM001282450.1 | OTOF/NM194248.2 | VEZT/NM017599.3 |
| ESRRA/NM004451.4 | OTOG/NM001292063.1 | VGLL4/NM014667.3 |
| ESRRB/NM004452.3 | OTOGL/NM173591.3 | VHL/NM000551.3 |
| ESRRG/NM001134285.2 | OTOL1/NM001080440.1 | VHLL/NM001004319.2 |
| ESRRG/NM001243509.1 | OTOP1/NM177998.1 | VIL1/NM007127.2 |
| ESRRG/NM001243512.1 | OTOP2/NM178160.2 | VILL/NM015873.3 |
| ESRRG/NM001243514.1 | OTOP3/NM178233.2 | VIM/NM003380.3 |
| ESRRG/NM206594.2 | OTOR/NM020157.3 | VIP/NM003381.3 |
| ESYT1/NM015292.2 | OTOS/NM148961.3 | VIPAS39/NM001193314.1 |
| ESYT2/NM020728.2 | OTP/NM032109.2 | VIPR1/NM004624.3 |
| ESYT3/NM031913.4 | OTUB1/NM017670.2 | VIPR2/NM003382.4 |
| ETF1/NM004730.3 | OTUB2/NM023112.3 | VIT/NM053276.3 |
| ETFA/NM000126.3 | OTUD1/NM001145373.2 | VKORC1/NM024006.5 |
| ETFB/NM001985.2 | OTUD3/NM015207.1 | VKORC1L1/NM173517.4 |
| ETFBKMT/NM001135863.1 | OTUD6B/NM016023.3 | VLDLR/NM003383.4 |
| ETFBKMT/NM001135864.1 | OTUD7A/NM130901.1 | VMAC/NM001017921.3 |
| ETFBKMT/NM173802.3 | OTUD7B/NM020205.3 | VMO1/NM182566.2 |
| ETFDH/NM004453.3 | OTULIN/NM138348.4 | VMP1/NM030938.3 |
| ETHE1/NM014297.4 | OTX2/NM001270525.1 | VN1R1/NM020633.3 |
| ETNK1/NM018638.4 | OVCA2/NM080822.2 | VN1R2/NM173856.2 |
| ETNK2/NM018208.3 | OVCH1/NM183378.2 | VN1R4/NM173857.2 |
| ETS1/NM005238.3 | OVCH2/NM198185.4 | VN1R5/NM173858.1 |
| ETS2/NM005239.5 | OVGP1/NM002557.3 | VNN1/NM004666.2 |
| ETV2/NM014209.3 | OVOLI/NM004561.3 | VNN2/NM004665.4 |
| ETV3/NM001145312.1 | OVOL2/NM021220.3 | VNN3/NM001291702.1 |
| ETV3L/NM001004341.2 | OVOL3/NM001302757.1 | VOPP1/NM030796.4 |
| ETV4/NM001079675.2 | OXA1L/NM005015.3 | VPRBP/NM014703.2 |
| ETV4/NM001986.2 | OXCT1/NM000436.3 | VPREB1/NM007128.3 |
| ETV5/NM004454.2 | OXCT2/NM022120.1 | VPREB3/NM013378.2 |
| ETV6/NM001987.4 | OXER1/NM148962.4 | VPS11/NM021729.5 |
| ETV7/NM016135.3 | OXGR1/NM080818.3 | VPS13A/NM033305.2 |
| EVA1A/NM001135032.1 | OXLD1/NM001039842.2 | VPS13B/NM017890.4 |
| EVA1A/NM032181.2 | OXNAD1/NM138381.3 | VPS13D/NM015378.3 |
| EVA1B/NM001304762.1 | OXR1/NM181354.4 | VPS16/NM022575.3 |
| EVA1B/NM018166.2 | OXSM/NM017897.2 | VPS26A/NM004896.4 |
| EVA1C/NM058187.4 | OXSR1/NM005109.2 | VPS26B/NM052875.4 |
| EVC/NM153717.2 | OXT/NM000915.3 | VPS28/NM016208.3 |
| EVC2/NM147127.4 | OXTR/NM000916.3 | VPS29/NM016226.4 |
| EVI2A/NM001003927.2 | P2RX1/NM002558.3 | VPS33A/NM022916.4 |
| EVI2B/NM006495.3 | P2RX2/NM170683.3 | VPS33B/NM018668.4 |
| EVI5L/NM145245.4 | P2RX3/NM002559.3 | VPS35/NM018206.5 |
| EVL/NM016337.2 | P2RX4/NM002560.2 | VPS37A/NM152415.2 |
| EVPL/NM001988.3 | P2RX5/NM002561.3 | VPS37B/NM024667.2 |
| EVPLL/NM001145127.1 | P2RX6/NM005446.3 | VPS37C/NM017966.4 |
| EVX1/NM001989.4 | P2RX7/NM002562.5 | VPS37D/NM001077621.1 |
| EVX2/NM001080458.1 | P2RY1/NM002563.4 | VPS39/NM015289.3 |
| EWSR1/NM013986.3 | P2RY11/NM002566.4 | VPS41/NM014396.3 |
| EXD2/NM018199.3 | P2RY12/NM022788.4 | VPS45/NM007259.4 |
| EXD3/NM017820.4 | P2RY12/NM176876.2 | VPS4A/NM013245.2 |
| EXO1/NM006027.4 | P2RY13/NM176894.2 | VPS4B/NM004869.3 |
| EXO1/NM130398.3 | P2RY14/NM001081455.1 | VPS51/NM013265.3 |
| EXOC1/NM018261.3 | P2RY14/NM014879.3 | VPS52/NM022553.5 |
| EXOC2/NM018303.5 | P2RY2/NM002564.3 | VPS54/NM016516.2 |
| EXOC3/NM007277.4 | P2RY2/NM176071.2 | VPS72/NM005997.2 |
| EXOC3L1/NM178516.3 | P2RY6/NM001277204.1 | VPS8/NM001009921.2 |
| EXOC3L2/NM138568.3 | P2RY6/NM001277206.1 | VPS9D1/NM004913.2 |
| EXOC3L4/NM001077594.1 | P2RY6/NM001277207.1 | VRK1/NM003384.2 |
| EXOC4/NM021807.3 | P2RY6/NM176797.2 | VRK2/NM001130480.2 |
| EXOC5/NM006544.3 | P2RY6/NM176798.2 | VRK2/NM001130481.2 |
| EXOC6/NM019053.5 | P3H1/NM022356.3 | VRK2/NM001288837.1 |
| EXOC6B/NM015189.2 | P3H2/NM018192.3 | VRK3/NM016440.3 |
| EXOC7/NM015219.4 | P3H3/NM014262.4 | VRTN/NM018228.2 |
| EXOC8/NM175876.4 | P3H4/NM006455.2 | VSIG10/NM019086.5 |
| EXOG/NM005107.3 | P4HA1/NM000917.3 | VSIG10L/NM001163922.1 |
| EXOSC1/NM016046.4 | P4HA2/NM001142599.1 | VSIG2/NM014312.3 |
| EXOSC10/NM001001998.2 | P4HA2/NM004199.2 | VSIG8/NM001013661.1 |
| EXOSC2/NM014285.6 | P4HA3/NM182904.4 | VSNL1/NM003385.4 |
| EXOSC4/NM019037.2 | P4HB/NM000918.3 | VSTM2B/NM001146339.1 |
| EXOSC5/NM020158.3 | P4HTM/NM177939.2 | VSTM2L/NM080607.2 |
| EXOSC6/NM058219.2 | PA2G4/NM006191.2 | VSTM4/NM144984.3 |
| EXOSC7/NM015004.3 | PAAF1/NM025155.2 | VSTM5/NM001144871.1 |
| EXOSC8/NM181503.2 | PABPC1/NM002568.3 | VSX1/NM014588.5 |
| EXOSC9/NM005033.2 | PABPC3/NM030979.2 | VSX2/NM182894.2 |
| EXPH5/NM015065.2 | PABPC4/NM003819.3 | VTA1/NM016485.4 |
| EXT1/NM000127.2 | PABPC4L/NM001114734.1 | VTCN1/NM024626.3 |
| EXTL1/NM004455.2 | PABPN1/NM004643.3 | VTI1A/NM145206.3 |
| EXTL2/NM001439.3 | PABPN1L/NM001080487.2 | VTN/NM000638.3 |
| EXTL3/NM001440.3 | PACRG/NM001080378.1 | VWA1/NM022834.4 |
| EYA1/NM000503.5 | PACRG/NM001080379.1 | VWA2/NM001272046.1 |
| EYA1/NM172058.3 | PACRGL/NM001258345.1 | VWA3A/NM173615.4 |
| EYA2/NM005244.4 | PACS1/NM018026.3 | VWA3B/NM144992.4 |
| EYA3/NM001990.3 | PACS2/NM015197.3 | VWA5A/NM014622.4 |
| EYA4/NM004100.4 | PACSIN1/NM001199583.2 | VWA5B2/NM138345.2 |
| EYS/NM001142800.1 | PACSIN1/NM020804.4 | VWA7/NM025258.2 |
| EZH1/NM001991.4 | PACSIN2/NM001184970.1 | VWA8/NM001009814.1 |
| EZH2/NM004456.4 | PACSIN2/NM007229.3 | VWC2/NM198570.4 |
| EZR/NM001111077.1 | PACSIN3/NM001184975.1 | VWCE/NM152718.2 |
| EZR/NM003379.4 | PACSIN3/NM016223.4 | VWF/NM000552.4 |
| F10/NM000504.3 | PADI2/NM007365.2 | WAC/NM016628.4 |
| F11/NM000128.3 | PADI3/NM016233.2 | WAPL/NM015045.3 |
| F11R/NM016946.4 | PADI4/NM012387.2 | WARS/NM004184.3 |
| F12/NM000505.3 | PADI6/NM207421.4 | WARS/NM173701.1 |
| F13A1/NM000129.3 | PAF1/NM019088.3 | WARS2/NM015836.3 |
| F13B/NM001994.2 | PAFAH1B1/NM000430.3 | WASF1/NM003931.2 |
| F2R/NM001992.4 | PAFAH1B2/NM002572.3 | WASF2/NM006990.4 |
| F2RL1/NM005242.4 | PAFAH1B3/NM001145939.1 | WASF3/NM006646.5 |
| F2RL2/NM004101.3 | PAFAH1B3/NM001145940.1 | WASH1/NM182905.4 |
| F2RL3/NM003950.2 | PAFAH1B3/NM002573.3 | WASL/NM003941.3 |
| F3/NM001993.4 | PAFAH2/NM000437.3 | WBP1/NM012477.3 |
| F5/NM000130.4 | PAG1/NM018440.3 | WBP11/NM016312.2 |
| F7/NM000131.4 | PAGR1/NM024516.3 | WBP1L/NM017787.4 |
| FA2H/NM024306.4 | PAH/NM000277.1 | WBP2/NM012478.3 |
| FAAH/NM001441.2 | PAICS/NM001079524.1 | WBP2NL/NM152613.2 |
| FAAP100/NM025161.5 | PAICS/NM006452.3 | WBP4/NM007187.3 |
| FAAP20/NM182533.2 | PAIP1/NM183323.2 | WBSCR17/NM022479.2 |
| FABP1/NM001443.2 | PAIP2/NM001033112.2 | WBSCR22/NM017528.4 |
| FABP12/NM001105281.1 | PAIP2/NM016480.4 | WBSCR27/NM152559.2 |
| FABP2/NM000134.3 | PAIP2B/NM020459.1 | WBSCR28/NM182504.3 |
| FABP3/NM004102.4 | PAK1/NM002576.4 | WDCP/NM025203.2 |
| FABP4/NM001442.2 | PAK1IP1/NM017906.2 | WDFY1/NM020830.4 |
| FABP5/NM001444.2 | PAK2/NM002577.4 | WDFY3/NM014991.4 |
| FABP6/NM001445.2 | PAK4/NM001014832.1 | WDFY4/NM020945.1 |
| FABP9/NM001080526.1 | PAK6/NM001276717.1 | WDHD1/NM007086.3 |
| FADD/NM003824.3 | PAK6/NM020168.5 | WDPCP/NM015910.5 |
| FADS1/NM013402.4 | PALB2/NM024675.3 | WDR1/NM017491.3 |
| FADS2/NM004265.3 | PALD1/NM014431.2 | WDR11/NM018117.11 |
| FADS3/NM021727.4 | PALLD/NM016081.3 | WDR12/NM018256.3 |
| FADS6/NM178128.5 | PALM/NM002579.2 | WDR18/NM024100.3 |
| FAF1/NM007051.2 | PALM2-AKAP2/NM007203.4 | WDR19/NM025132.3 |
| FAF2/NM014613.2 | PALM3/NM001145028.1 | WDR20/NM181291.2 |
| FAH/NM000137.2 | PALMD/NM017734.4 | WDR24/NM032259.3 |
| FAHD1/NM031208.3 | PAM/NM138766.2 | WDR25/NM001161476.1 |
| FAHD2A/NM016044.2 | PAM16/NM016069.9 | WDR25/NM024515.4 |
| FAHD2B/NM001320848.1 | PAMR1/NM001001991.2 | WDR26/NM025160.6 |
| FAIM/NM001033032.1 | PAN2/NM014871.4 | WDR27/NM182552.4 |
| FAIM/NM018147.3 | PAN3/NM175854.7 | WDR3/NM006784.2 |
| FAIM2/NM012306.3 | PANK1/NM148978.2 | WDR31/NM145241.4 |
| FAM101A/NM181709.4 | PANK2/NM153638.3 | WDR33/NM018383.4 |
| FAM101B/NM182705.2 | PANK3/NM024594.3 | WDR34/NM052844.3 |
| FAM102A/NM001035254.2 | PANK4/NM018216.2 | WDR35/NM020779.3 |
| FAM102B/NM001010883.2 | PANO1/NM001293167.1 | WDR36/NM139281.2 |
| FAM103A1/NM031452.3 | PANX1/NM015368.3 | WDR37/NM014023.3 |
| FAM104A/NM032837.2 | PANX2/NM052839.3 | WDR4/NM033661.4 |
| FAM105A/NM019018.2 | PANX3/NM052959.2 | WDR41/NM018268.3 |
| FAM107A/NM007177.3 | PAOX/NM152911.3 | WDR43/NM015131.2 |
| FAM107B/NM031453.3 | PAPD4/NM001114393.1 | WDR45B/NM019613.3 |
| FAM109A/NM144671.4 | PAPD5/NM001040284.2 | WDR46/NM005452.5 |
| FAM109B/NM001002034.2 | PAPD7/NM006999.4 | WDR47/NM014969.5 |
| FAM110A/NM001289146.1 | PAPLN/NM173462.3 | WDR48/NM020839.3 |
| FAM110A/NM001289147.1 | PAPOLA/NM032632.4 | WDR49/NM178824.3 |
| FAM110A/NM031424.5 | PAPOLB/NM020144.4 | WDR5/NM017588.2 |
| FAM110A/NM207121.4 | PAPOLG/NM022894.3 | WDR5/NM052821.3 |
| FAM110B/NM147189.2 | PAPPA/NM002581.4 | WDR53/NM182627.1 |
| FAM110C/NM001077710.2 | PAPSS1/NM005443.4 | WDR54/NM032118.3 |
| FAM110D/NM024869.2 | PAPSS2/NM004670.3 | WDR55/NM017706.4 |
| FAM111A/NM001142519.2 | PAQR3/NM001040202.1 | WDR59/NM030581.3 |
| FAM111A/NM198847.2 | PAQR4/NM152341.4 | WDR5B/NM019069.3 |
| FAM111B/NM198947.3 | PAQR5/NM001104554.1 | WDR6/NM018031.4 |
| FAM114A1/NM138389.2 | PAQR5/NM017705.3 | WDR60/NM018051.4 |
| FAM117A/NM030802.3 | PAQR6/NM001272104.1 | WDR61/NM001303247.1 |
| FAM117B/NM173511.3 | PAQR7/NM178422.5 | WDR62/NM173636.4 |
| FAM118A/NM001104595.1 | PAQR8/NM133367.4 | WDR63/NM145172.4 |
| FAM118A/NM017911.2 | PAQR9/NM198504.2 | WDR64/NM144625.4 |
| FAM118B/NM024556.3 | PARD3/NM019619.3 | WDR66/NM144668.5 |
| FAM120A/NM014612.4 | PARD3B/NM001302769.1 | WDR7/NM015285.2 |
| FAM120AOS/NM198841.2 | PARD6B/NM032521.2 | WDR70/NM018034.2 |
| FAM120B/NM032448.2 | PARD6G/NM032510.3 | WDR72/NM182758.3 |
| FAM122A/NM138333.4 | PARG/NM003631.3 | WDR73/NM032856.3 |
| FAM124A/NM145019.3 | PARK2/NM004562.2 | WDR74/NM018093.3 |
| FAM124B/NM024785.2 | PARK7/NM001123377.1 | WDR75/NM032168.2 |
| FAM126A/NM032581.3 | PARL/NM018622.6 | WDR76/NM024908.3 |
| FAM126B/NM173822.4 | PARM1/NM015393.3 | WDR77/NM024102.3 |
| FAM129A/NM052966.3 | PARP1/NM001618.3 | WDR78/NM024763.4 |
| FAM129B/NM022833.3 | PARP10/NM032789.4 | WDR81/NM001163809.1 |
| FAM129C/NM173544.4 | PARP11/NM020367.5 | WDR82/NM025222.3 |
| FAM131A/NM144635.4 | PARP12/NM022750.3 | WDR83/NM001099737.2 |
| FAM131B/NM001031690.2 | PARP14/NM017554.2 | WDR83/NM032332.3 |
| FAM131C/NM182623.2 | PARP15/NM001113523.2 | WDR83OS/NM016145.3 |
| FAM132A/NM001014980.2 | PARP16/NM017851.5 | WDR86/NM198285.2 |
| FAM132B/NM001291832.1 | PARP2/NM005484.3 | WDR88/NM173479.3 |
| FAM133B/NM152789.3 | PARP3/NM005485.5 | WDR89/NM001258272.1 |
| FAM134A/NM024293.5 | PARP4/NM006437.3 | WDR90/NM145294.4 |
| FAM134B/NM019000.4 | PARP6/NM020214.3 | WDR91/NM014149.3 |
| FAM134C/NM178126.3 | PARP8/NM001178055.1 | WDR93/NM020212.1 |
| FAM135A/NM001105531.2 | PARP8/NM024615.3 | WDR97/NM001316309.1 |
| FAM135B/NM015912.3 | PARP9/NM001146102.1 | WDSUB1/NM001128212.2 |
| FAM136A/NM032822.2 | PARP9/NM031458.2 | WDSUB1/NM001128213.1 |
| FAM13A/NM014883.3 | PARPBP/NM017915.4 | WDSUB1/NM152528.2 |
| FAM13B/NM016603.3 | PARS2/NM152268.3 | WDTC1/NM015023.4 |
| FAM149A/NM001006655.2 | PARVA/NM018222.4 | WDYHV1/NM018024.2 |
| FAM149A/NM015398.2 | PARVB/NM013327.4 | WEE1/NM003390.3 |
| FAM149B1/NM173348.1 | PARVG/NM001137605.2 | WEE2/NM001105558.1 |
| FAM150B/NM001002919.2 | PARVG/NM022141.6 | WFDC1/NM001282466.1 |
| FAM151A/NM176782.2 | PATE1/NM138294.2 | WFDC10A/NM080753.2 |
| FAM151B/NM205548.2 | PATE2/NM212555.2 | WFDC10B/NM172131.2 |
| FAM153B/NM001265615.1 | PATE3/NM001129883.3 | WFDC11/NM147197.2 |
| FAM155A/NM001080396.2 | PATE4/NM001144874.1 | WFDC12/NM080869.1 |
| FAM157A/NM001145248.1 | PATL1/NM152716.2 | WFDC13/NM172005.1 |
| FAM157B/NM001145249.1 | PATL2/NM001145112.1 | WFDC2/NM006103.3 |
| FAM159A/NM001042693.2 | PATZ1/NM014323.2 | WFDC3/NM080614.1 |
| FAM159B/NM001164442.1 | PAWR/NM002583.2 | WFDC5/NM145652.3 |
| FAM160A1/NM001109977.1 | PAX1/NM006192.4 | WFDC6/NM080827.1 |
| FAM160A2/NM032127.3 | PAX2/NM001304569.1 | WFDC8/NM181510.2 |
| FAM160B1/NM020940.3 | PAX4/NM006193.2 | WFDC9/NM147198.3 |
| FAM160B2/NM022749.5 | PAX5/NM016734.2 | WFIKKN1/NM053284.2 |
| FAM161A/NM032180.2 | PAX6/NM001258462.1 | WFIKKN2/NM175575.5 |
| FAM161B/NM152445.2 | PAX6/NM001258463.1 | WFS1/NM001145853.1 |
| FAM162A/NM014367.3 | PAX6/NM001604.5 | WHAMM/NM001080435.2 |
| FAM162B/NM001085480.2 | PAX7/NM001135254.1 | WHRN/NM015404.3 |
| FAM163A/NM173509.2 | PAX9/NM006194.3 | WHSC1/NM001042424.2 |
| FAM163B/NM001080515.2 | PAXBP1/NM016631.3 | WHSC1/NM133331.2 |
| FAM166A/NM001001710.2 | PBK/NM018492.3 | WHSC1L1/NM023034.1 |
| FAM166B/NM001164310.2 | PBLD/NM022129.3 | WI2-2373I1.2/NM001195127.1 |
| FAM167A/NM053279.2 | PBOV1/NM021635.2 | WIF1/NM007191.4 |
| FAM167B/NM032648.2 | PBX1/NM002585.3 | WIPF1/NM001077269.1 |
| FAM168B/NM001321743.1 | PBX2/NM002586.4 | WIPF1/NM003387.4 |
| FAM168B/NM001321747.1 | PBX3/NM006195.5 | WIPF2/NM133264.4 |
| FAM168B/NM001321748.1 | PBX4/NM025245.2 | WIPF3/NM001080529.2 |
| FAM169A/NM015566.2 | PBXIP1/NM020524.3 | WIPI1/NM017983.6 |
| FAM169B/NM182562.2 | PC/NM001040716.1 | WIPI2/NM016003.3 |
| FAM170A/NM182761.3 | PC/NM022172.2 | WISP1/NM003882.3 |
| FAM170B/NM001164484.1 | PCBD1/NM000281.3 | WISP3/NM198239.1 |
| FAM171A1/NM001010924.1 | PCBD2/NM032151.4 | WLS/NM024911.6 |
| FAM171A2/NM198475.2 | PCBP1/NM006196.3 | WNK1/NM018979.3 |
| FAM171B/NM177454.3 | PCBP2/NM031989.4 | WNK2/NM001282394.1 |
| FAM172A/NM032042.5 | PCBP3/NM020528.2 | WNK4/NM032387.4 |
| FAM173A/NM023933.2 | PCBP4/NM001174100.1 | WNT1/NM005430.3 |
| FAM173B/NM199133.3 | PCBP4/NM033010.2 | WNT10A/NM025216.2 |
| FAM174A/NM198507.2 | PCCA/NM000282.3 | WNT10B/NM003394.3 |
| FAM174B/NM207446.2 | PCCB/NM000532.4 | WNT11/NM004626.2 |
| FAM175B/NM032182.3 | PCDH1/NM032420.3 | WNT16/NM057168.1 |
| FAM177A1/NM173607.3 | PCDH12/NM016580.3 | WNT2/NM003391.2 |
| FAM177B/NM001324080.1 | PCDH15/NM033056.3 | WNT2B/NM024494.2 |
| FAM177B/NM207468.2 | PCDH17/NM001040429.2 | WNT3/NM030753.4 |
| FAM178B/NM001122646.2 | PCDH18/NM019035.4 | WNT3A/NM033131.3 |
| FAM179A/NM199280.2 | PCDH20/NM022843.3 | WNT4/NM030761.4 |
| FAM179B/NM001308120.1 | PCDH7/NM001173523.1 | WNT5A/NM003392.4 |
| FAM180A/NM205855.3 | PCDH8/NM002590.3 | WNT5B/NM030775.2 |
| FAM180B/NM001164379.1 | PCDH9/NM203487.2 | WNT5B/NM032642.2 |
| FAM181B/NM175885.3 | PCDHA10/NM018901.3 | WNT6/NM006522.3 |
| FAM183A/NM001101376.2 | PCDHA11/NM018902.4 | WNT7A/NM004625.3 |
| FAM184A/NM024581.5 | PCDHA12/NM018903.3 | WNT7B/NM058238.2 |
| FAM184B/NM015688.1 | PCDHA13/NM018904.2 | WNT8A/NM058244.3 |
| FAM185A/NM001145268.1 | PCDHA2/NM018905.2 | WNT8B/NM003393.3 |
| FAM186A/NM001145475.1 | PCDHA3/NM018906.2 | WNT9A/NM003395.2 |
| FAM186B/NM032130.2 | PCDHA4/NM018907.3 | WNT9B/NM003396.2 |
| FAM187B/NM152481.1 | PCDHA5/NM018908.2 | WRAP53/NM001143990.1 |
| FAM188A/NM024948.3 | PCDHA6/NM018909.3 | WRAP53/NM001143991.1 |
| FAM188B/NM032222.2 | PCDHA7/NM018910.2 | WRAP53/NM001143992.1 |
| FAM189A1/NM015307.1 | PCDHA8/NM018911.2 | WRAP73/NM017818.3 |
| FAM189B/NM006589.2 | PCDHA9/NM031857.1 | WRB-SH3BGR/NM001317744.1 |
| FAM193A/NM003704.3 | PCDHAC1/NM018898.3 | WRB/NM004627.4 |
| FAM193B/NM001190946.2 | PCDHAC2/NM018899.5 | WRN/NM000553.4 |
| FAM196A/NM001039762.2 | PCDHB1/NM013340.3 | WRNIP1/NM020135.2 |
| FAM196B/NM001129891.1 | PCDHB10/NM018930.3 | WSB1/NM015626.8 |
| FAM198A/NM001129908.2 | PCDHB11/NM018931.2 | WSB2/NM018639.4 |
| FAM198B/NM001128424.1 | PCDHB12/NM018932.3 | WSCD1/NM015253.1 |
| FAM198B/NM016613.6 | PCDHB13/NM018933.3 | WSCD2/NM001304447.1 |
| FAM19A1/NM001252216.1 | PCDHB14/NM018934.3 | WSCD2/NM014653.3 |
| FAM19A1/NM213609.3 | PCDHB15/NM018935.3 | WT1/NM024426.4 |
| FAM19A2/NM178539.4 | PCDHB16/NM020957.3 | WTAP/NM001270531.1 |
| FAM19A3/NM182759.2 | PCDHB2/NM018936.3 | WTAP/NM004906.4 |
| FAM19A5/NM001082967.2 | PCDHB3/NM018937.4 | WTH3DI/NM001077637.1 |
| FAM200A/NM145111.3 | PCDHB4/NM018938.3 | WTIP/NM001080436.1 |
| FAM200B/NM001145191.1 | PCDHB5/NM015669.4 | WWC1/NM015238.2 |
| FAM204A/NM022063.2 | PCDHB7/NM018940.3 | WWC2/NM024949.5 |
| FAM205A/NM001141917.1 | PCDHB8/NM019120.4 | WWOX/NM016373.3 |
| FAM205C/NM001309426.1 | PCDHB9/NM019119.4 | WWP1/NM007013.3 |
| FAM206A/NM017832.3 | PCDHGA1/NM018912.2 | WWP2/NM001270454.1 |
| FAM207A/NM001316984.1 | PCDHGA10/NM018913.2 | WWTR1/NM001168278.1 |
| FAM208A/NM001112736.1 | PCDHGA11/NM018914.2 | WWTR1/NM001168280.1 |
| FAM209A/NM001012971.3 | PCDHGA12/NM003735.2 | WWTR1/NM015472.4 |
| FAM209B/NM001013646.3 | PCDHGA2/NM018915.3 | XAF1/NM017523.3 |
| FAM20A/NM017565.3 | PCDHGA3/NM018916.3 | XBP1/NM005080.3 |
| FAM20B/NM001324310.1 | PCDHGA4/NM018917.3 | XCL1/NM002995.2 |
| FAM20B/NM001324311.1 | PCDHGA5/NM018918.2 | XCL2/NM003175.3 |
| FAM20B/NM014864.3 | PCDHGA6/NM018919.2 | XCR1/NM005283.2 |
| FAM20C/NM020223.3 | PCDHGA7/NM018920.3 | XDH/NM000379.3 |
| FAM210B/NM080821.2 | PCDHGA8/NM032088.1 | XIRP1/NM194293.2 |
| FAM212A/NM203370.1 | PCDHGA9/NM018921.2 | XIRP2/NM152381.5 |
| FAM212B/NM019099.4 | PCDHGB1/NM018922.2 | XKR3/NM001318251.1 |
| FAM213A/NM001243778.1 | PCDHGB2/NM018923.2 | XKR4/NM052898.1 |
| FAM213A/NM001243779.1 | PCDHGB3/NM018924.4 | XKR5/NM207411.4 |
| FAM213A/NM032333.4 | PCDHGB4/NM003736.2 | XKR6/NM173683.3 |
| FAM213B/NM152371.3 | PCDHGB5/NM018925.2 | XKR7/NM001011718.1 |
| FAM214A/NM019600.3 | PCDHGB6/NM018926.2 | XKR8/NM018053.3 |
| FAM214B/NM001317991.1 | PCDHGB7/NM018927.3 | XKR9/NM001011720.1 |
| FAM214B/NM025182.3 | PCDHGC3/NM002588.3 | XKR9/NM001287260.1 |
| FAM217A/NM173563.2 | PCDHGC4/NM018928.2 | XPA/NM000380.3 |
| FAM217B/NM001190826.1 | PCDHGC5/NM018929.2 | XPC/NM004628.4 |
| FAM217B/NM022106.2 | PCED1A/NM022760.4 | XPNPEP1/NM020383.3 |
| FAM218A/NM153027.1 | PCED1B/NM001281429.1 | XPNPEP3/NM022098.3 |
| FAM219A/NM147202.1 | PCF11/NM015885.3 | XPO1/NM003400.3 |
| FAM219B/NM020447.4 | PCGF1/NM032673.2 | XPO4/NM022459.4 |
| FAM21A/NM001005751.2 | PCGF2/NM007144.2 | XPO5/NM020750.2 |
| FAM21C/NM015262.2 | PCGF3/NM006315.5 | XPO6/NM015171.3 |
| FAM220A/NM001037163.1 | PCGF5/NM001256549.1 | XPO7/NM015024.4 |
| FAM221A/NM199136.4 | PCGF5/NM001257101.1 | XPR1/NM004736.3 |
| FAM221B/NM001012446.3 | PCGF5/NM032373.4 | XRCC1/NM006297.2 |
| FAM222A/NM032829.2 | PCGF6/NM001011663.1 | XRCC2/NM005431.1 |
| FAM222B/NM001288632.1 | PCID2/NM001127202.3 | XRCC3/NM001100119.1 |
| FAM222B/NM001288634.1 | PCIF1/NM022104.3 | XRCC4/NM022406.3 |
| FAM227B/NM152647.2 | PCK1/NM002591.3 | XRCC5/NM021141.3 |
| FAM228A/NM001040710.2 | PCK2/NM004563.3 | XRCC6/NM001288976.1 |
| FAM228B/NM001145710.1 | PCLO/NM033026.5 | XRCC6/NM001469.4 |
| FAM229A/NM001167676.1 | PCM1/NM006197.3 | XRN1/NM019001.4 |
| FAM229B/NM001033564.2 | PCMT1/NM001252053.1 | XRN2/NM012255.4 |
| FAM231A/NM001282321.1 | PCMTD1/NM052937.3 | XRRA1/NM182969.2 |
| FAM231B/NM001310155.1 | PCMTD2/NM018257.2 | XXYLT1/NM152531.4 |
| FAM234B/NM020853.1 | PCNA/NM182649.1 | XYLB/NM005108.3 |
| FAM24A/NM001029888.1 | PCNT/NM006031.5 | XYLT1/NM022166.3 |
| FAM24B/NM152644.2 | PCNX1/NM014982.2 | XYLT2/NM022167.3 |
| FAM25A/NM001146157.2 | PCNX2/NM014801.3 | YAE1D1/NM020192.4 |
| FAM25C/NM001137548.2 | PCNX3/NM032223.3 | YAF2/NM005748.5 |
| FAM25G/NM001137549.1 | PCNX4/NM022495.5 | YAP1/NM001130145.2 |
| FAM26E/NM153711.3 | PCOLCE/NM002593.3 | YARS/NM003680.3 |
| FAM26F/NM001010919.2 | PCOLCE2/NM013363.3 | YARS2/NM001040436.2 |
| FAM35A/NM019054.2 | PCP2/NM001271830.1 | YBEY/NM001314025.1 |
| FAM3B/NM058186.3 | PCP4/NM006198.2 | YBX1/NM004559.4 |
| FAM3C/NM014888.2 | PCP4L1/NM001102566.1 | YBX2/NM015982.3 |
| FAM3D/NM138805.2 | PCSK1/NM000439.4 | YBX3/NM003651.4 |
| FAM43A/NM153690.4 | PCSK5/NM006200.5 | YDJC/NM001017964.1 |
| FAM43B/NM207334.2 | PCSK6/NM002570.4 | YEATS2/NM018023.4 |
| FAM45A/NM207009.3 | PCSK7/NM004716.3 | YEATS4/NM006530.3 |
| FAM46A/NM017633.2 | PCSK9/NM174936.3 | YES1/NM005433.3 |
| FAM46B/NM052943.3 | PCTP/NM021213.3 | YIF1A/NM020470.2 |
| FAM46C/NM017709.3 | PCYOX1/NM016297.3 | YIF1B/NM001039672.2 |
| FAM47E-STBD1/NM001242939.1 | PCYOX1L/NM024028.3 | YIPF1/NM018982.4 |
| FAM49A/NM030797.3 | PCYT1A/NM005017.3 | YIPF2/NM001321439.1 |
| FAM50B/NM012135.2 | PCYT2/NM002861.4 | YIPF2/NM024029.4 |
| FAM53A/NM001013622.3 | PDAP1/NM014891.6 | YIPF3/NM015388.3 |
| FAM53A/NM001174070.2 | PDC/NM002597.4 | YIPF4/NM032312.3 |
| FAM53B/NM014661.3 | PDCD1/NM005018.2 | YIPF7/NM182592.2 |
| FAM57A/NM024792.2 | PDCD10/NM007217.3 | YJEFN3/NM198537.3 |
| FAM57B/NM031478.5 | PDCD10/NM145859.1 | YKT6/NM006555.3 |
| FAM60A/NM001135811.1 | PDCD10/NM145860.1 | YLPM1/NM019589.2 |
| FAM60A/NM001135812.1 | PDCD11/NM014976.1 | YME1L1/NM014263.3 |
| FAM63B/NM001040450.2 | PDCD1LG2/NM025239.3 | YOD1/NM018566.3 |
| FAM64A/NM019013.2 | PDCD2/NM002598.3 | YPEL1/NM013313.4 |
| FAM65A/NM024519.3 | PDCD2L/NM032346.1 | YPEL2/NM001005404.3 |
| FAM65B/NM014722.3 | PDCD4/NM014456.4 | YPEL3/NM001145524.1 |
| FAM65C/NM080829.3 | PDCD5/NM004708.3 | YPEL4/NM145008.2 |
| FAM69A/NM001006605.4 | PDCD6/NM013232.3 | YPEL5/NM016061.2 |
| FAM69B/NM152421.3 | PDCD6IP/NM013374.5 | YRDC/NM024640.3 |
| FAM69C/NM001044369.2 | PDCD7/NM005707.1 | YTHDC1/NM133370.2 |
| FAM71A/NM153606.3 | PDCL/NM005388.4 | YTHDC2/NM022828.3 |
| FAM71B/NM130899.2 | PDCL2/NM152401.2 | YTHDF1/NM017798.3 |
| FAM71C/NM153364.3 | PDCL3/NM024065.4 | YTHDF2/NM001173128.1 |
| FAM71D/NM173526.3 | PDE10A/NM001130690.2 | YTHDF2/NM016258.2 |
| FAM71E1/NM138411.2 | PDE11A/NM016953.3 | YTHDF3/NM001277813.1 |
| FAM71E2/NM001145402.1 | PDE12/NM177966.6 | YTHDF3/NM001277814.1 |
| FAM71F1/NM032599.3 | PDE1A/NM005019.4 | YWHAB/NM139323.3 |
| FAM71F2/NM001012454.5 | PDE1B/NM000924.3 | YWHAE/NM006761.4 |
| FAM72A/NM001123168.2 | PDE1C/NM001191056.3 | YWHAG/NM012479.3 |
| FAM72B/NM001100910.1 | PDEIC/NM005020.4 | YWHAH/NM003405.3 |
| FAM72C/NM001287385.1 | PDE2A/NM002599.4 | YWHAQ/NM006826.3 |
| FAM72D/NM207418.2 | PDE3A/NM000921.4 | YWHAZ/NM001135699.1 |
| FAM73B/NM032809.2 | PDE3B/NM000922.3 | YWHAZ/NM001135701.1 |
| FAM76A/NM152660.2 | PDE4A/NM001111307.1 | YWHAZ/NM001135702.1 |
| FAM76B/NM144664.4 | PDE4B/NM001037341.1 | YWHAZ/NM003406.3 |
| FAM78A/NM033387.3 | PDE4B/NM002600.3 | YWHAZ/NM145690.2 |
| FAM78B/NM001017961.4 | PDE4C/NM000923.4 | YY1/NM003403.4 |
| FAM81A/NM152450.2 | PDE4D/NM001165899.1 | YY1AP1/NM139118.2 |
| FAM81B/NM152548.2 | PDE4DIP/NM001198834.3 | ZACN/NM180990.3 |
| FAM83A/NM032899.6 | PDE5A/NM033430.2 | ZADH2/NM175907.5 |
| FAM83B/NM001010872.2 | PDE6A/NM000440.2 | ZAK/NM016653.2 |
| FAM83C/NM178468.5 | PDE6B/NM000283.3 | ZAN/NM003386.2 |
| FAM83D/NM030919.2 | PDE6C/NM006204.3 | ZAP70/NM001079.3 |
| FAM83E/NM017708.3 | PDE6D/NM002601.3 | ZAR1/NM175619.2 |
| FAM83F/NM138435.2 | PDE6G/NM002602.3 | ZAR1L/NM001136571.1 |
| FAM83G/NM001039999.2 | PDE6H/NM006205.2 | ZASP/NM001289933.1 |
| FAM83H/NM198488.3 | PDE7A/NM002603.3 | ZBBX/NM024687.3 |
| FAM84A/NM145175.2 | PDE7B/NM018945.3 | ZBED2/NM024508.4 |
| FAM84B/NM174911.4 | PDE8A/NM002605.2 | ZBED3/NM032367.2 |
| FAM86B1/NM001083537.1 | PDE8B/NM003719.3 | ZBED4/NM014838.2 |
| FAM86B2/NM001137610.1 | PDE9A/NM002606.2 | ZBED5/NM021211.3 |
| FAM86C1/NM001099653.1 | PDF/NM022341.1 | ZBED6/NM001174108.1 |
| FAM89A/NM198552.2 | PDGFA/NM033023.4 | ZBED6CL/NM138434.2 |
| FAM89B/NM001098785.1 | PDGFB/NM002608.3 | ZBED8/NM001303251.1 |
| FAM8A1/NM016255.2 | PDGFC/NM016205.2 | ZBED9/NM052923.1 |
| FAM90A1/NM018088.3 | PDGFD/NM033135.3 | ZBP1/NM030776.2 |
| FAM91A1/NM144963.3 | PDGFRA/NM006206.4 | ZBTB1/NM001123329.1 |
| FAM92A1/NM145269.4 | PDGFRB/NM002609.3 | ZBTB10/NM001105539.2 |
| FAM96A/NM032231.5 | PDGFRL/NM006207.2 | ZBTB11/NM014415.3 |
| FAM96B/NM016062.3 | PDHA2/NM005390.4 | ZBTB12/NM181842.2 |
| FAM98A/NM015475.4 | PDHB/NM000925.3 | ZBTB14/NM001143823.2 |
| FAM98B/NM173611.3 | PDHX/NM003477.2 | ZBTB14/NM001243702.1 |
| FAM98C/NM174905.3 | PDIA2/NM006849.2 | ZBTB14/NM001243704.1 |
| FAN1/NM014967.4 | PDIA3/NM005313.4 | ZBTB17/NM003443.2 |
| FANCA/NM000135.2 | PDIA4/NM004911.4 | ZBTB18/NM205768.2 |
| FANCC/NM000136.2 | PDIA5/NM006810.3 | ZBTB2/NM020861.2 |
| FANCC/NM001243743.1 | PDIA6/NM005742.3 | ZBTB20/NM001164343.2 |
| FANCD2/NM033084.4 | PDIK1L/NM001243532.1 | ZBTB20/NM001164344.2 |
| FANCD2OS/NM173472.1 | PDIKIL/NM152835.4 | ZBTB20/NM001164346.2 |
| FANCE/NM021922.2 | PDILT/NM174924.1 | ZBTB20/NM001164347.2 |
| FANCF/NM022725.3 | PDK1/NM002610.4 | ZBTB20/NM015642.5 |
| FANCG/NM004629.1 | PDK2/NM002611.4 | ZBTB21/NM001320731.1 |
| FANCI/NM018193.2 | PDK4/NM002612.3 | ZBTB22/NM001145338.1 |
| FANCL/NM018062.3 | PDLIM1/NM020992.3 | ZBTB22/NM005453.4 |
| FANK1/NM145235.3 | PDLIM2/NM021630.5 | ZBTB24/NM014797.2 |
| FAR1/NM032228.5 | PDLIM3/NM014476.5 | ZBTB25/NM001304507.1 |
| FAR2/NM001271783.1 | PDLIM4/NM003687.3 | ZBTB25/NM006977.3 |
| FAR2/NM018099.4 | PDLIM5/NM006457.4 | ZBTB26/NM001304363.1 |
| FARP1/NM005766.3 | PDLIM7/NM005451.4 | ZBTB26/NM001304364.1 |
| FARP2/NM014808.3 | PDP1/NM001161781.1 | ZBTB26/NM020924.3 |
| FARS2/NM001318872.1 | PDP1/NM018444.3 | ZBTB3/NM024784.3 |
| FARS2/NM006567.4 | PDP2/NM020786.2 | ZBTB32/NM014383.2 |
| FARSA/NM004461.2 | PDPK1/NM002613.4 | ZBTB37/NM001122770.1 |
| FARSB/NM005687.4 | PDPN/NM006474.4 | ZBTB38/NM001080412.2 |
| FAS/NM000043.5 | PDPR/NM001322117.1 | ZBTB39/NM014830.2 |
| FASLG/NM000639.2 | PDRG1/NM030815.2 | ZBTB4/NM020899.3 |
| FASN/NM004104.4 | PDS5A/NM001100399.1 | ZBTB40/NM014870.3 |
| FASTK/NM006712.4 | PDS5B/NM015032.3 | ZBTB41/NM194314.2 |
| FASTKD1/NM024622.5 | PDSS1/NM014317.4 | ZBTB42/NM001137601.2 |
| FASTKD2/NM014929.3 | PDSS2/NM020381.3 | ZBTB43/NM001135776.1 |
| FASTKD3/NM024091.3 | PDX1/NM000209.3 | ZBTB45/NM001316978.1 |
| FASTKD5/NM021826.4 | PDXDC1/NM015027.3 | ZBTB45/NM001316980.1 |
| FAT1/NM005245.3 | PDXK/NM003681.4 | ZBTB45/NM001316981.1 |
| FAT2/NM001447.2 | PDXP/NM020315.4 | ZBTB45/NM001316982.1 |
| FAT4/NM024582.4 | PDYN/NM001190892.1 | ZBTB46/NM025224.3 |
| FAU/NM001997.4 | PDYN/NM001190900.1 | ZBTB47/NM145166.3 |
| FAXC/NM032511.2 | PDYN/NM024411.4 | ZBTB48/NM001278647.1 |
| FAXDC2/NM032385.4 | PDZD2/NM178140.3 | ZBTB49/NM145291.3 |
| FBF1/NM001319193.1 | PDZD3/NM001168468.1 | ZBTB5/NM014872.2 |
| FBL/NM001436.3 | PDZD7/NM001195263.1 | ZBTB6/NM006626.5 |
| FBLIM1/NM017556.2 | PDZD8/NM173791.4 | ZBTB7A/NM015898.3 |
| FBLN1/NM006486.2 | PDZK1/NM002614.4 | ZBTB7B/NM001256455.1 |
| FBLN2/NM001004019.1 | PDZK1IP1/NM005764.3 | ZBTB7C/NM001039360.2 |
| FBLN2/NM001165035.1 | PDZRN3/NM015009.2 | ZBTB7C/NM001318841.1 |
| FBLN5/NM006329.3 | PDZRN4/NM001164595.1 | ZBTB8A/NM001040441.2 |
| FBLN7/NM153214.2 | PEA15/NM003768.4 | ZBTB8B/NM001145720.1 |
| FBN1/NM000138.4 | PEAK1/NM024776.3 | ZBTB8OS/NM178547.3 |
| FBN2/NM001999.3 | PEAR1/NM001080471.1 | ZBTB9/NM152735.3 |
| FBN3/NM032447.4 | PEBP1/NM002567.3 | ZC2HC1A/NM016010.2 |
| FBP1/NM000507.3 | PEBP4/NM144962.2 | ZC2HC1B/NM001013623.2 |
| FBP1/NM001127628.1 | PECAM1/NM000442.4 | ZC2HC1C/NM024643.3 |
| FBP2/NM003837.3 | PECR/NM018441.5 | ZC3H10/NM001303125.1 |
| FBRSL1/NM001142641.1 | PEF1/NM012392.3 | ZC3H10/NM032786.2 |
| FBXL12/NM017703.2 | PEG10/NM001040152.1 | ZC3H11A/NM014827.5 |
| FBXL13/NM145032.3 | PEG3/NM001146186.1 | ZC3H12A/NM001323550.1 |
| FBXL14/NM152441.2 | PEG3/NM006210.2 | ZC3H12D/NM207360.2 |
| FBXL15/NM024326.3 | PELI2/NM021255.2 | ZC3H13/NM015070.4 |
| FBXL16/NM153350.3 | PELI3/NM001098510.1 | ZC3H14/NM024824.4 |
| FBXL17/NM001163315.2 | PELO/NM015946.4 | ZC3H15/NM018471.2 |
| FBXL18/NM024963.5 | PELP1/NM014389.2 | ZC3H18/NM144604.3 |
| FBXL19/NM001099784.2 | PEMT/NM007169.2 | ZC3H3/NM015117.2 |
| FBXL2/NM012157.3 | PEMT/NM148173.1 | ZC3H4/NM015168.1 |
| FBXL20/NM032875.2 | PENK/NM001135690.2 | ZC3H6/NM198581.2 |
| FBXL21/NM012159.4 | PEPD/NM000285.3 | ZC3H7A/NM014153.3 |
| FBXL22/NM203373.2 | PER1/NM002616.2 | ZC3H7B/NM017590.5 |
| FBXL3/NM012158.2 | PER2/NM022817.2 | ZC3H8/NM032494.2 |
| FBXL4/NM001278716.1 | PERM1/NM001291366.1 | ZC3HAV1/NM020119.3 |
| FBXL5/NM012161.3 | PERP/NM022121.4 | ZC3HAV1L/NM080660.3 |
| FBXL6/NM012162.3 | PES1/NM014303.3 | ZC3HC1/NM016478.4 |
| FBXL7/NM012304.4 | PET117/NM001164811.1 | ZCCHC10/NM017665.3 |
| FBXL8/NM018378.2 | PEX1/NM000466.2 | ZCCHC11/NM001009881.2 |
| FBXO10/NM012166.2 | PEX10/NM002617.3 | ZCCHC14/NM015144.2 |
| FBXO11/NM025133.4 | PEX11A/NM003847.2 | ZCCHC17/NM016505.3 |
| FBXO16/NM172366.3 | PEX11B/NM003846.2 | ZCCHC2/NM017742.5 |
| FBXO17/NM024907.6 | PEX11G/NM080662.3 | ZCCHC24/NM153367.3 |
| FBXO18/NM032807.4 | PEX12/NM000286.2 | ZCCHC3/NM033089.6 |
| FBXO2/NM012168.5 | PEX13/NM002618.3 | ZCCHC4/NM024936.2 |
| FBXO21/NM033624.2 | PEX14/NM004565.2 | ZCCHC6/NM024617.3 |
| FBXO22/NM147188.2 | PEX16/NM004813.2 | ZCCHC7/NM001289120.1 |
| FBXO24/NM012172.4 | PEX19/NM002857.3 | ZCCHC7/NM032226.2 |
| FBXO25/NM183421.1 | PEX2/NM000318.2 | ZCCHC8/NM017612.4 |
| FBXO27/NM178820.4 | PEX2/NM001172086.1 | ZCCHC9/NM001131036.1 |
| FBXO28/NM015176.3 | PEX26/NM001127649.2 | ZCRB1/NM033114.3 |
| FBXO3/NM012175.3 | PEX26/NM017929.5 | ZCWPW1/NM017984.4 |
| FBXO30/NM032145.4 | PEX3/NM003630.2 | ZCWPW2/NM001040432.3 |
| FBXO31/NM024735.4 | PEX5/NM001131025.1 | ZDBF2/NM020923.2 |
| FBXO32/NM058229.3 | PEX5/NM001131026.1 | ZDHHC1/NM013304.2 |
| FBXO33/NM203301.3 | PEX5L/NM016559.2 | ZDHHC11/NM024786.2 |
| FBXO34/NM017943.3 | PEX6/NM000287.3 | ZDHHC14/NM024630.2 |
| FBXO34/NM152231.1 | PEX7/NM000288.3 | ZDHHC16/NM032327.3 |
| FBXO36/NM174899.4 | PF4/NM002619.3 | ZDHHC18/NM032283.2 |
| FBXO38/NM030793.4 | PF4V1/NM002620.3 | ZDHHC19/NM001039617.1 |
| FBXO39/NM153230.2 | PFAS/NM012393.2 | ZDHHC2/NM016353.4 |
| FBXO40/NM016298.3 | PFDN1/NM002622.4 | ZDHHC21/NM178566.4 |
| FBXO41/NM001080410.2 | PFDN6/NM001185181.2 | ZDHHC22/NM174976.2 |
| FBXO42/NM018994.2 | PFDN6/NM001265596.1 | ZDHHC23/NM173570.4 |
| FBXO43/NM001029860.3 | PFKFB2/NM006212.2 | ZDHHC24/NM207340.1 |
| FBXO44/NM001304791.1 | PFKFB4/NM004567.3 | ZDHHC3/NM001135179.1 |
| FBXO44/NM033182.5 | PFKM/NM000289.5 | ZDHHC4/NM001134389.1 |
| FBXO45/NM001105573.1 | PFKP/NM002627.4 | ZDHHC6/NM022494.2 |
| FBXO46/NM001080469.1 | PFN1/NM005022.3 | ZDHHC7/NM017740.2 |
| FBXO47/NM001008777.2 | PFN2/NM053024.3 | ZDHHC8/NM013373.3 |
| FBXO48/NM001024680.2 | PFN3/NM001029886.2 | ZEB2/NM014795.3 |
| FBXO5/NM012177.3 | PFN4/NM199346.2 | ZFAND1/NM024699.2 |
| FBXO6/NM018438.5 | PGA3/NM001079807.3 | ZFAND2A/NM182491.2 |
| FBXO7/NM012179.3 | PGA4/NM001079808.3 | ZFAND3/NM021943.2 |
| FBXO8/NM012180.2 | PGA5/NM014224.4 | ZFAND4/NM001128324.2 |
| FBXO9/NM033480.2 | PGAM1/NM002629.3 | ZFAND4/NM174890.3 |
| FBXW10/NM001267585.1 | PGAM2/NM000290.3 | ZFAND5/NM001102420.2 |
| FBXW11/NM012300.2 | PGAM5/NM001170543.1 | ZFAND5/NM001278243.1 |
| FBXW12/NM207102.2 | PGAP1/NM024989.3 | ZFAND6/NM001242911.1 |
| FBXW2/NM012164.3 | PGAP2/NM014489.3 | ZFAND6/NM001242913.1 |
| FBXW4/NM022039.3 | PGBD1/NM032507.3 | ZFAND6/NM001242914.1 |
| FBXW5/NM018998.3 | PGBD2/NM170725.2 | ZFAND6/NM001242915.1 |
| FBXW7/NM018315.4 | PGBD3/NM170753.3 | ZFAT/NM001029939.3 |
| FBXW8/NM153348.2 | PGBD4/NM152595.4 | ZFAT/NM001289394.1 |
| FBXW9/NM032301.2 | PGBD5/NM001258311.1 | ZFC3H1/NM144982.4 |
| FCAMR/NM032029.4 | PGC/NM002630.3 | ZFHX2/NM033400.2 |
| FCAR/NM002000.3 | PGD/NM002631.3 | ZFHX3/NM006885.3 |
| FCER1A/NM002001.3 | PGF/NM002632.5 | ZFHX4/NM024721.4 |
| FCER1G/NM004106.1 | PGGT1B/NM005023.3 | ZFP1/NM153688.3 |
| FCER2/NM001220500.1 | PGK2/NM138733.4 | ZFP14/NM020917.2 |
| FCF1/NM015962.4 | PGLS/NM012088.2 | ZFP2/NM030613.3 |
| FCGBP/NM003890.2 | PGLYRP1/NM005091.2 | ZFP28/NM020828.1 |
| FCGR1A/NM000566.3 | PGLYRP2/NM052890.3 | ZFP3/NM153018.2 |
| FCGR1B/NM001244910.1 | PGLYRP3/NM052891.2 | ZFP30/NM014898.2 |
| FCGR2A/NM021642.3 | PGLYRP4/NM020393.3 | ZFP36/NM003407.3 |
| FCGR2B/NM004001.4 | PGM1/NM002633.2 | ZFP36L1/NM004926.3 |
| FCGR3A/NM000569.6 | PGM2/NM018290.3 | ZFP37/NM003408.2 |
| FCGR3B/NM000570.4 | PGM2L1/NM173582.4 | ZFP41/NM001271156.2 |
| FCGRT/NM001136019.2 | PGM5/NM021965.3 | ZFP41/NM173832.5 |
| FCGRT/NM004107.4 | PGP/NM001042371.2 | ZFP42/NM001304358.1 |
| FCHO1/NM001161358.1 | PGPEP1/NM017712.3 | ZFP57/NM001109809.2 |
| FCHO1/NM015122.2 | PGR/NM000926.4 | ZFP62/NM152283.4 |
| FCHO2/NM138782.2 | PGRMC2/NM006320.4 | ZFP64/NM199427.2 |
| FCHSD1/NM033449.2 | PHACTR1/NM001242648.2 | ZFP69/NM198494.2 |
| FCHSD2/NM014824.2 | PHACTR2/NM001100166.1 | ZFP69B/NM023070.2 |
| FCMR/NM005449.4 | PHACTR3/NM080672.4 | ZFP82/NM133466.3 |
| FCN1/NM002003.3 | PHACTR4/NM023923.3 | ZFP90/NM133458.3 |
| FCN2/NM004108.2 | PHAX/NM032177.3 | ZFP91/NM053023.4 |
| FCN3/NM003665.2 | PHB/NM001281496.1 | ZFPL1/NM006782.3 |
| FCRL1/NM052938.4 | PHB/NM001281715.1 | ZFPM1/NM153813.2 |
| FCRL2/NM030764.3 | PHB2/NM001144831.1 | ZFPM2/NM012082.3 |
| FCRL3/NM052939.3 | PHC1/NM004426.2 | ZFR/NM016107.3 |
| FCRL4/NM031282.2 | PHC2/NM198040.2 | ZFR2/NM015174.1 |
| FCRL5/NM031281.2 | PHC3/NM024947.3 | ZFYVE1/NM021260.3 |
| FCRL6/NM001004310.2 | PHF1/NM024165.2 | ZFYVE16/NM014733.4 |
| FCRLA/NM032738.3 | PHF10/NM018288.3 | ZFYVE19/NM001077268.1 |
| FCRLB/NM001002901.3 | PHF11/NM001040443.2 | ZFYVE21/NM024071.3 |
| FDCSP/NM152997.3 | PHF12/NM001033561.1 | ZFYVE26/NM015346.3 |
| FDFT1/NM001287743.1 | PHF13/NM153812.2 | ZFYVE27/NM144588.6 |
| FDFT1/NM004462.4 | PHF19/NM015651.2 | ZFYVE28/NM020972.2 |
| FDPS/NM001135821.1 | PHF2/NM005392.3 | ZFYVE9/NM004799.3 |
| FDPS/NM002004.3 | PHF20/NM016436.4 | ZG16/NM152338.3 |
| FDX1/NM004109.4 | PHF20L1/NM016018.4 | ZG16B/NM145252.2 |
| FDX1L/NM001031734.3 | PHF21A/NM016621.3 | ZGLP1/NM001103167.1 |
| FDXACB1/NM138378.2 | PHF23/NM024297.2 | ZGPAT/NM032527.4 |
| FDXR/NM024417.4 | PHF3/NM015153.3 | ZGRF1/NM018392.4 |
| FECH/NM000140.3 | PHF5A/NM032758.3 | ZHX1-C8orf76/NM001204180.1 |
| FEM1A/NM018708.2 | PHF7/NM001321126.1 | ZHX2/NM014943.3 |
| FEM1B/NM015322.4 | PHGDH/NM006623.3 | ZHX3/NM015035.3 |
| FEM1C/NM020177.2 | PHGR1/NM001145643.1 | ZIC2/NM007129.3 |
| FER1L5/NM001293083.1 | PHIP/NM017934.5 | ZIC4/NM032153.5 |
| FER1L6/NM001039112.2 | PHKB/NM000293.2 | ZIC5/NM033132.3 |
| FERD3L/NM152898.2 | PHKG1/NM006213.4 | ZIK1/NM001010879.3 |
| FERMT1/NM017671.4 | PHKG2/NM000294.2 | ZIM2/NM001146327.1 |
| FERMT2/NM006832.2 | PHLDA1/NM007350.3 | ZIM3/NM052882.1 |
| FERMT3/NM031471.5 | PHLDA2/NM003311.3 | ZKSCAN1/NM003439.2 |
| FES/NM002005.3 | PHLDA3/NM012396.4 | ZKSCAN2/NM001012981.4 |
| FETUB/NM014375.2 | PHLDB1/NM001144758.2 | ZKSCAN3/NM024493.3 |
| FEV/NM017521.2 | PHLDB1/NM015157.3 | ZKSCAN4/NM019110.4 |
| FEZ1/NM005103.4 | PHLDB2/NM145753.2 | ZKSCAN5/NM014569.3 |
| FEZ2/NM001042548.1 | PHLDB3/NM198850.3 | ZKSCAN5/NM145102.3 |
| FEZF2/NM018008.3 | PHLPP1/NM194449.3 | ZKSCAN7/NM001288590.1 |
| FFAR1/NM005303.2 | PHOSPHO1/NM178500.3 | ZKSCAN8/NM006298.3 |
| FFAR2/NM005306.2 | PHOSPHO2-KLHL23/NM001199290.1 | ZMAT2/NM144723.2 |
| FFAR3/NM005304.3 | PHOX2A/NM005169.3 | ZMAT3/NM022470.3 |
| FFAR4/NM001195755.1 | PHOX2B/NM003924.3 | ZMAT4/NM024645.2 |
| FGA/NM000508.4 | PHPT1/NM014172.5 | ZMAT5/NM001318129.1 |
| FGD2/NM173558.3 | PHRF1/NM020901.3 | ZMAT5/NM019103.2 |
| FGD3/NM001083536.1 | PHTF1/NM001323041.1 | ZMIZ1/NM020338.3 |
| FGD3/NM033086.2 | PHTF1/NM001323042.1 | ZMIZ2/NM031449.3 |
| FGD4/NM139241.3 | PHTF1/NM006608.2 | ZMPSTE24/NM005857.4 |
| FGD5/NM152536.3 | PHTF2/NM001127357.1 | ZMYM1/NM001289088.1 |
| FGD6/NM018351.3 | PHYH/NM006214.3 | ZMYM1/NM024772.4 |
| FGF1/NM000800.4 | PHYHD1/NM001100876.1 | ZMYM2/NM001190964.2 |
| FGF1/NM001144892.2 | PHYHIP/NM001099335.1 | ZMYM2/NM001190965.2 |
| FGF1/NM001257205.1 | PHYKPL/NM153373.3 | ZMYM2/NM197968.3 |
| FGF1/NM001257209.1 | PI15/NM015886.4 | ZMYM4/NM005095.2 |
| FGF1/NM001257210.1 | PI16/NM001199159.1 | ZMYM5/NM001142684.1 |
| FGF10/NM004465.1 | PI16/NM153370.2 | ZMYM6/NM007167.3 |
| FGF11/NM004112.3 | PI3/NM002638.3 | ZMYND10/NM015896.3 |
| FGF12/NM021032.4 | PI4K2A/NM018425.3 | ZMYND11/NM006624.5 |
| FGF14/NM175929.2 | PI4K2B/NM018323.3 | ZMYND12/NM032257.4 |
| FGF17/NM003867.3 | PI4KA/NM058004.3 | ZMYND15/NM032265.2 |
| FGF18/NM003862.2 | PI4KB/NM001198773.1 | ZMYND19/NM138462.2 |
| FGF19/NM005117.2 | PI4KB/NM001198774.1 | ZMYND8/NM183047.3 |
| FGF2/NM002006.4 | PIANP/NM001244015.1 | ZNF10/NM015394.4 |
| FGF20/NM019851.2 | PIANP/NM153685.3 | ZNF100/NM173531.3 |
| FGF21/NM019113.3 | PIAS1/NM016166.2 | ZNF101/NM033204.3 |
| FGF22/NM020637.1 | PIAS2/NM004671.4 | ZNF107/NM001282359.1 |
| FGF23/NM020638.2 | PIAS3/NM006099.3 | ZNF112/NM013380.3 |
| FGF3/NM005247.2 | PIAS4/NM015897.3 | ZNF114/NM153608.2 |
| FGF4/NM002007.2 | PIBF1/NM006346.2 | ZNF117/NM015852.3 |
| FGF5/NM004464.3 | PICALM/NM001008660.2 | ZNF121/NM001308269.1 |
| FGF6/NM020996.2 | PICK1/NM001039584.1 | ZNF124/NM001297568.1 |
| FGF7/NM002009.3 | PID1/NM001100818.1 | ZNF131/NM003432.2 |
| FGF8/NM033163.3 | PIDD1/NM145886.3 | ZNF132/NM003433.3 |
| FGF9/NM002010.2 | PIEZO1/NM001142864.3 | ZNF133/NM001083330.2 |
| FGFBP1/NM005130.4 | PIEZO2/NM022068.3 | ZNF134/NM003435.4 |
| FGFBP2/NM031950.3 | PIF1/NM001286496.1 | ZNF135/NM003436.3 |
| FGFBP3/NM152429.4 | PIFO/NM181643.5 | ZNF136/NM003437.3 |
| FGFR1/NM023110.2 | PIGB/NM004855.4 | ZNF138/NM006524.3 |
| FGFR1OP/NM007045.3 | PIGBOS1/NM001308421.1 | ZNF14/NM021030.2 |
| FGFR3/NM000142.4 | PIGF/NM002643.3 | ZNF140/NM003440.3 |
| FGFR4/NM002011.4 | PIGG/NM017733.4 | ZNF141/NM003441.2 |
| FGFRL1/NM001004356.2 | PIGH/NM004569.3 | ZNF142/NM001105537.2 |
| FGFRL1/NM001004358.1 | PIGK/NM005482.2 | ZNF146/NM001099639.1 |
| FGFRL1/NM021923.3 | PIGL/NM004278.3 | ZNF148/NM021964.2 |
| FGGY/NM018291.3 | PIGM/NM145167.2 | ZNF154/NM001085384.2 |
| FGL1/NM201552.1 | PIGN/NM176787.4 | ZNF155/NM001260487.1 |
| FGL1/NM201553.1 | PIGO/NM032634.3 | ZNF16/NM001029976.2 |
| FGL2/NM006682.2 | PIGP/NM001320480.1 | ZNF160/NM001102603.1 |
| FGR/NM001042729.1 | PIGQ/NM148920.2 | ZNF165/NM003447.3 |
| FGR/NM001042747.1 | PIGR/NM002644.3 | ZNF169/NM194320.3 |
| FGR/NM005248.2 | PIGS/NM033198.3 | ZNF17/NM006959.2 |
| FH/NM000143.3 | PIGT/NM015937.5 | ZNF175/NM007147.3 |
| FHAD1/NM052929.1 | PIGU/NM080476.4 | ZNF177/NM003451.2 |
| FHDC1/NM033393.2 | PIGV/NM001202554.1 | ZNF18/NM001303281.1 |
| FHIT/NM001320900.1 | PIGV/NM017837.3 | ZNF18/NM144680.3 |
| FHL2/NM001039492.2 | PIGW/NM178517.3 | ZNF180/NM013256.5 |
| FHL2/NM001318894.1 | PIGX/NM017861.3 | ZNF181/NM001029997.3 |
| FHL2/NM201555.1 | PIGZ/NM025163.3 | ZNF184/NM001318891.1 |
| FHL2/NM201557.3 | PIH1D1/NM017916.2 | ZNF184/NM001318892.1 |
| FHL3/NM004468.4 | PIH1D2/NM138789.3 | ZNF189/NM003452.3 |
| FHL5/NM001170807.2 | PIK3AP1/NM152309.2 | ZNF19/NM006961.3 |
| FHL5/NM001322466.1 | PIK3C2A/NM002645.3 | ZNF195/NM007152.4 |
| FHL5/NM001322467.1 | PIK3C2B/NM002646.3 | ZNF197/NM006991.3 |
| FHOD1/NM013241.2 | PIK3C2G/NM004570.5 | ZNF2/NM021088.3 |
| FHOD3/NM025135.4 | PIK3C3/NM002647.3 | ZNF20/NM021143.3 |
| FIBCD1/NM001145106.1 | PIK3CA/NM006218.3 | ZNF200/NM003454.3 |
| FIBCD1/NM032843.4 | PIK3CB/NM006219.2 | ZNF200/NM198088.2 |
| FIBIN/NM203371.1 | PIK3CD/NM005026.3 | ZNF202/NM001301779.1 |
| FIBP/NM004214.4 | PIK3CG/NM001282427.1 | ZNF205/NM001042428.1 |
| FICD/NM007076.2 | PIK3CG/NM002649.3 | ZNF205/NM001278158.1 |
| FIG4/NM014845.5 | PIK3IP1/NM052880.4 | ZNF207/NM003457.3 |
| FIGLA/NM001004311.3 | PIK3R1/NM181523.2 | ZNF211/NM006385.4 |
| FIGN/NM018086.3 | PIK3R2/NM005027.3 | ZNF212/NM012256.3 |
| FIGNL2/NM001013690.4 | PIK3R3/NM001114172.1 | ZNF213/NM001134655.1 |
| FILIP1/NM015687.3 | PIK3R3/NM003629.3 | ZNF213/NM004220.2 |
| FILIP1L/NM182909.3 | PIK3R4/NM014602.2 | ZNF214/NM013249.2 |
| FIS1/NM016068.2 | PIK3R5/NM014308.3 | ZNF215/NM013250.2 |
| FITM1/NM203402.2 | PIK3R6/NM001010855.3 | ZNF217/NM006526.2 |
| FITM2/NM001080472.2 | PIKFYVE/NM015040.3 | ZNF219/NM001101672.1 |
| FIZ1/NM032836.2 | PILRA/NM013439.2 | ZNF219/NM001102454.1 |
| FJX1/NM014344.3 | PILRB/NM178238.3 | ZNF219/NM016423.2 |
| FKBP10/NM021939.3 | PIM1/NM002648.3 | ZNF22/NM006963.4 |
| FKBP11/NM016594.2 | PIM3/NM001001852.3 | ZNF221/NM001297588.1 |
| FKBP14/NM017946.3 | PIN1/NM006221.3 | ZNF221/NM013359.2 |
| FKBP15/NM015258.1 | PINK1/NM032409.2 | ZNF222/NM013360.2 |
| FKBP1A/NM000801.4 | PINLYP/NM001193621.1 | ZNF223/NM013361.5 |
| FKBP1B/NM004116.4 | PINX1/NM017884.5 | ZNF224/NM013398.3 |
| FKBP2/NM001135208.1 | PIP/NM002652.2 | ZNF225/NM013362.3 |
| FKBP2/NM004470.3 | PIP4K2A/NM005028.4 | ZNF226/NM001032372.1 |
| FKBP2/NM057092.2 | PIP4K2B/NM003559.4 | ZNF226/NM001032373.1 |
| FKBP4/NM002014.3 | PIP4K2C/NM001146258.1 | ZNF226/NM001319089.1 |
| FKBP5/NM001145775.2 | PIP5K1B/NM003558.3 | ZNF226/NM016444.2 |
| FKBP5/NM001145776.1 | PIP5K1C/NM012398.2 | ZNF227/NM182490.2 |
| FKBP5/NM004117.3 | PIP5KL1/NM001135219.1 | ZNF229/NM014518.3 |
| FKBP6/NM003602.4 | PIPOX/NM016518.2 | ZNF23/NM001304492.1 |
| FKBP7/NM181342.2 | PIRT/NM001101387.1 | ZNF23/NM145911.2 |
| FKBP8/NM012181.4 | PISD/NM014338.3 | ZNF230/NM006300.3 |
| FKBP9/NM007270.4 | PITHD1/NM020362.4 | ZNF232/NM014519.3 |
| FKBPL/NM022110.3 | PITPNA/NM006224.3 | ZNF233/NM001207005.1 |
| FKRP/NM024301.4 | PITPNC1/NM012417.3 | ZNF233/NM181756.2 |
| FKTN/NM006731.2 | PITPNM1/NM004910.2 | ZNF234/NM001144824.1 |
| FLAD1/NM201398.2 | PITPNM2/NM020845.2 | ZNF235/NM004234.4 |
| FLCN/NM144997.5 | PITPNM3/NM031220.3 | ZNF236/NM007345.3 |
| FLG/NM002016.1 | PITRM1/NM001242307.1 | ZNF239/NM001099282.1 |
| FLG2/NM001014342.2 | PITX1/NM002653.4 | ZNF239/NM001099284.1 |
| FLI1/NM002017.4 | PITX2/NM000325.5 | ZNF239/NM005674.2 |
| FLII/NM002018.3 | PITX3/NM005029.3 | ZNF24/NM006965.3 |
| FLJ45513/NM001242791.1 | PIWIL2/NM001135721.1 | ZNF25/NM145011.2 |
| FLNB/NM001457.3 | PIWIL2/NM018068.3 | ZNF250/NM021061.4 |
| FLNC/NM001127487.1 | PIWIL3/NM001008496.3 | ZNF251/NM138367.1 |
| FLOT1/NM005803.3 | PIWIL4/NM152431.2 | ZNF253/NM021047.2 |
| FLOT2/NM004475.2 | PJA2/NM014819.4 | ZNF254/NM203282.3 |
| FLRT1/NM013280.4 | PKD1/NM001009944.2 | ZNF256/NM005773.2 |
| FLRT2/NM013231.4 | PKD1L1/NM138295.3 | ZNF257/NM033468.3 |
| FLRT3/NM198391.2 | PKD1L2/NM052892.3 | ZNF26/NM019591.3 |
| FLT1/NM002019.4 | PKD1L3/NM181536.1 | ZNF260/NM001012756.2 |
| FLT3/NM004119.2 | PKD2/NM000297.3 | ZNF263/NM005741.4 |
| FLT3LG/NM001204503.1 | PKD2L1/NM016112.2 | ZNF264/NM003417.4 |
| FLT3LG/NM001459.3 | PKD2L2/NM001300921.1 | ZNF267/NM003414.5 |
| FLT4/NM182925.4 | PKDCC/NM138370.2 | ZNF268/NM001165881.2 |
| FLVCR1/NM014053.3 | PKDREJ/NM006071.1 | ZNF268/NM003415.2 |
| FLVCR2/NM017791.2 | PKIA/NM181839.2 | ZNF273/NM021148.2 |
| FLYWCH1/NM032296.2 | PKIB/NM032471.5 | ZNF274/NM133502.2 |
| FLYWCH2/NM001142499.1 | PKIB/NM181794.2 | ZNF276/NM152287.3 |
| FMN1/NM001103184.3 | PKIG/NM001281445.1 | ZNF277/NM021994.2 |
| FMN2/NM020066.4 | PKIG/NM007066.4 | ZNF28/NM006969.3 |
| FMNL1/NM005892.3 | PKLR/NM181871.3 | ZNF280A/NM080740.4 |
| FMNL2/NM052905.3 | PKM/NM002654.5 | ZNF280B/NM080764.3 |
| FMNL3/NM175736.4 | PKMYT1/NM004203.4 | ZNF281/NM001281293.1 |
| FMO1/NM002021.2 | PKN1/NM213560.1 | ZNF284/NM001037813.2 |
| FMO4/NM002022.2 | PKN2/NM006256.3 | ZNF285/NM001291489.1 |
| FMO5/NM001461.3 | PKN3/NM013355.4 | ZNF286A/NM001130842.1 |
| FMOD/NM002023.4 | PKNOX1/NM004571.4 | ZNF286B/NM001145045.1 |
| FN1/NM212474.2 | PKNOX2/NM022062.2 | ZNF287/NM020653.2 |
| FN3K/NM022158.3 | PKP2/NM001005242.2 | ZNF292/NM015021.1 |
| FN3KRP/NM024619.3 | PKP3/NM007183.3 | ZNF296/NM145288.1 |
| FNBP1/NM015033.2 | PKP4/NM003628.4 | ZNF3/NM001278284.1 |
| FNBP1L/NM001164473.2 | PLA1A/NM015900.3 | ZNF3/NM032924.4 |
| FNBP4/NM015308.3 | PLA2G10/NM003561.2 | ZNF30/NM001099437.1 |
| FNDC1/NM032532.2 | PLA2G12A/NM030821.4 | ZNF302/NM001289186.1 |
| FNDC3A/NM014923.4 | PLA2G12B/NM032562.4 | ZNF304/NM020657.3 |
| FNDC3B/NM001135095.1 | PLA2G15/NM012320.3 | ZNF311/NM001010877.3 |
| FNDC3B/NM022763.3 | PLA2G16/NM001128203.1 | ZNF316/NM001278559.1 |
| FNDC5/NM153756.2 | PLA2G16/NM007069.3 | ZNF317/NM020933.4 |
| FNDC7/NM001144937.1 | PLA2G1B/NM000928.2 | ZNF318/NM014345.2 |
| FNDC8/NM017559.2 | PLA2G2A/NM000300.3 | ZNF319/NM020807.2 |
| FNDC9/NM001001343.3 | PLA2G2A/NM001161729.1 | ZNF32/NM001005368.2 |
| FNIP1/NM133372.2 | PLA2G2C/NM001316722.1 | ZNF32/NM006973.2 |
| FNIP2/NM020840.2 | PLA2G2D/NM012400.3 | ZNF320/NM207333.2 |
| FNTA/NM002027.2 | PLA2G2E/NM014589.2 | ZNF322/NM001242797.1 |
| FNTB/NM002028.3 | PLA2G2F/NM022819.3 | ZNF324/NM014347.2 |
| FOCAD/NM017794.4 | PLA2G3/NM015715.4 | ZNF324B/NM207395.2 |
| FOLH1/NM004476.1 | PLA2G4A/NM024420.2 | ZNF326/NM182976.3 |
| FOLR1/NM016729.2 | PLA2G4C/NM003706.2 | ZNF329/NM024620.3 |
| FOLR2/NM001113536.1 | PLA2G4D/NM178034.3 | ZNF330/NM014487.5 |
| FOLR3/NM000804.3 | PLA2G4E/NM001206670.1 | ZNF331/NM001253799.1 |
| FOPNL/NM144600.3 | PLA2G4F/NM213600.3 | ZNF331/NM001253800.1 |
| FOS/NM005252.3 | PLA2G5/NM000929.2 | ZNF331/NM001253801.2 |
| FOSB/NM006732.2 | PLA2G6/NM003560.2 | ZNF331/NM001317113.1 |
| FOSL1/NM005438.4 | PLA2G7/NM001168357.1 | ZNF331/NM001317114.1 |
| FOSL2/NM005253.3 | PLA2G7/NM005084.3 | ZNF331/NM001317116.1 |
| FOXA1/NM004496.3 | PLA2R1/NM007366.4 | ZNF331/NM001317119.1 |
| FOXA2/NM153675.2 | PLAA/NM001031689.2 | ZNF331/NM001317120.1 |
| FOXA3/NM004497.2 | PLAC4/NM182832.2 | ZNF331/NM001317121.1 |
| FOXB1/NM012182.2 | PLAC8/NM001130716.1 | ZNF333/NM032433.2 |
| FOXB2/NM001013735.1 | PLAC8/NM016619.2 | ZNF334/NM018102.4 |
| FOXC1/NM001453.2 | PLAC8L1/NM001029869.1 | ZNF335/NM022095.3 |
| FOXC2/NM005251.2 | PLAC9/NM001012973.1 | ZNF337/NM001290261.1 |
| FOXD1/NM004472.2 | PLAG1/NM002655.2 | ZNF337/NM015655.3 |
| FOXD2/NM004474.3 | PLAGL1/NM001080951.2 | ZNF33A/NM006974.2 |
| FOXD3/NM012183.2 | PLAGL1/NM001289046.1 | ZNF33B/NM006955.2 |
| FOXD4/NM207305.4 | PLAGL1/NM001317156.1 | ZNF34/NM030580.4 |
| FOXD4L1/NM012184.4 | PLAGL1/NM001317157.1 | ZNF341/NM032819.4 |
| FOXD4L3/NM199135.4 | PLAGL1/NM001317159.1 | ZNF343/NM001282495.1 |
| FOXD4L4/NM199244.3 | PLAGL2/NM002657.3 | ZNF343/NM024325.5 |
| FOXD4L5/NM001126334.1 | PLAT/NM000930.4 | ZNF345/NM001242472.1 |
| FOXD4L6/NM001085476.1 | PLAU/NM002658.4 | ZNF345/NM001242475.1 |
| FOXE1/NM004473.3 | PLB1/NM153021.4 | ZNF345/NM003419.4 |
| FOXE3/NM012186.2 | PLBD1/NM024829.5 | ZNF346/NM012279.3 |
| FOXF1/NM001451.2 | PLCB1/NM015192.3 | ZNF347/NM032584.2 |
| FOXF2/NM001452.1 | PLCB2/NM004573.2 | ZNF35/NM003420.3 |
| FOXG1/NM005249.4 | PLCB3/NM001316314.1 | ZNF350/NM021632.3 |
| FOXH1/NM003923.2 | PLCB4/NM000933.3 | ZNF354A/NM005649.2 |
| FOXI1/NM012188.4 | PLCD1/NM006225.3 | ZNF354B/NM058230.2 |
| FOXI2/NM207426.2 | PLCD3/NM133373.4 | ZNF354C/NM014594.2 |
| FOXI3/NM001135649.1 | PLCD4/NM032726.3 | ZNF358/NM018083.4 |
| FOXJ1/NM001454.3 | PLCE1/NM016341.3 | ZNF362/NM152493.2 |
| FOXJ3/NM001198850.1 | PLCG1/NM002660.2 | ZNF365/NM014951.2 |
| FOXJ3/NM001198851.1 | PLCG2/NM002661.4 | ZNF366/NM152625.2 |
| FOXJ3/NM014947.4 | PLCH1/NM014996.2 | ZNF367/NM153695.3 |
| FOXK1/NM001037165.1 | PLCH2/NM014638.3 | ZNF37A/NM001324245.1 |
| FOXK2/NM004514.3 | PLCL1/NM006226.3 | ZNF382/NM032825.4 |
| FOXL1/NM005250.2 | PLCL2/NM015184.5 | ZNF383/NM152604.1 |
| FOXL2/NM023067.3 | PLCXD2/NM153268.3 | ZNF384/NM001135734.2 |
| FOXL2NB/NM001040061.2 | PLCXD3/NM001005473.2 | ZNF385A/NM001290002.1 |
| FOXM1/NM202002.2 | PLCZ1/NM033123.3 | ZNF385A/NM015481.2 |
| FOXN1/NM003593.2 | PLD1/NM002662.4 | ZNF385B/NM152520.4 |
| FOXN2/NM002158.3 | PLD2/NM002663.4 | ZNF391/NM001322288.1 |
| FOXN3/NM005197.3 | PLD3/NM001031696.3 | ZNF391/NM001322293.1 |
| FOXN4/NM213596.2 | PLD3/NM001291311.1 | ZNF394/NM032164.2 |
| FOXO1/NM002015.3 | PLD4/NM138790.3 | ZNF395/NM018660.2 |
| FOXO3/NM001455.3 | PLD5/NM152666.2 | ZNF396/NM001322290.1 |
| FOXO3/NM201559.2 | PLD6/NM178836.3 | ZNF397/NM001135178.2 |
| FOXO6/NM001291281.2 | PLEC/NM201380.3 | ZNF398/NM170686.2 |
| FOXP1/NM001244814.1 | PLEK2/NM016445.2 | ZNF404/NM001033719.2 |
| FOXP1/NM001244816.1 | PLEKHA1/NM001001974.2 | ZNF407/NM017757.2 |
| FOXP1/NM032682.5 | PLEKHA1/NM021622.4 | ZNF408/NM024741.2 |
| FOXP2/NM014491.3 | PLEKHA2/NM021623.1 | ZNF410/NM021188.2 |
| FOXP4/NM138457.2 | PLEKHA3/NM019091.3 | ZNF414/NM032370.2 |
| FOXQ1/NM033260.3 | PLEKHA4/NM020904.2 | ZNF415/NM001164309.1 |
| FOXR1/NM181721.2 | PLEKHA5/NM019012.5 | ZNF415/NM018355.3 |
| FOXRED1/NM017547.3 | PLEKHA7/NM175058.4 | ZNF416/NM017879.1 |
| FOXRED2/NM001102371.1 | PLEKHA8/NM001197026.1 | ZNF417/NM152475.2 |
| FOXRED2/NM024955.5 | PLEKHB1/NM021200.2 | ZNF418/NM133460.2 |
| FOXS1/NM004118.3 | PLEKHB2/NM017958.2 | ZNF419/NM001098494.1 |
| FPGS/NM004957.5 | PLEKHD1/NM001161498.1 | ZNF420/NM144689.3 |
| FPGT-TNNI3K/NM001112808.2 | PLEKHF1/NM024310.4 | ZNF425/NM001001661.2 |
| FPR1/NM001193306.1 | PLEKHF2/NM024613.3 | ZNF426/NM024106.2 |
| FPR2/NM001005738.1 | PLEKHG1/NM001029884.1 | ZNF429/NM001001415.2 |
| FPR2/NM001462.3 | PLEKHG2/NM022835.2 | ZNF43/NM003423.3 |
| FPR3/NM002030.4 | PLEKHG3/NM001308147.1 | ZNF430/NM025189.3 |
| FRA10AC1/NM145246.4 | PLEKHG4/NM001129727.2 | ZNF431/NM133473.2 |
| FRAT1/NM005479.3 | PLEKHG4/NM001129728.1 | ZNF432/NM001322284.1 |
| FRAT2/NM012083.2 | PLEKHG4/NM001129729.2 | ZNF432/NM001322285.1 |
| FREM1/NM144966.5 | PLEKHG4B/NM052909.3 | ZNF432/NM014650.3 |
| FREM2/NM207361.5 | PLEKHG5/NM001042664.1 | ZNF436/NM001077195.1 |
| FREM3/NM001168235.1 | PLEKHG5/NM001042665.1 | ZNF436/NM030634.2 |
| FRG1/NM004477.2 | PLEKHG5/NM020631.4 | ZNF438/NM001143766.1 |
| FRG2/NM001005217.1 | PLEKHG6/NM001144856.1 | ZNF439/NM152262.2 |
| FRG2B/NM001080998.1 | PLEKHG6/NM018173.3 | ZNF44/NM016264.3 |
| FRG2C/NM001124759.1 | PLEKHG7/NM001004330.2 | ZNF440/NM152357.2 |
| FRK/NM002031.2 | PLEKHH1/NM020715.2 | ZNF442/NM030824.2 |
| FRMD3/NM174938.5 | PLEKHH2/NM172069.3 | ZNF443/NM005815.4 |
| FRMD4A/NM018027.4 | PLEKHH3/NM024927.4 | ZNF444/NM018337.3 |
| FRMD4B/NM015123.2 | PLEKHJ1/NM018049.2 | ZNF445/NM181489.5 |
| FRMD5/NM032892.4 | PLEKHM1/NM014798.2 | ZNF446/NM017908.3 |
| FRMD6/NM001042481.2 | PLEKHM2/NM015164.2 | ZNF45/NM003425.3 |
| FRMD6/NM152330.3 | PLEKHM3/NM001080475.2 | ZNF451/NM001257273.1 |
| FRMD8/NM031904.3 | PLEKHN1/NM032129.2 | ZNF454/NM001178090.2 |
| FRMPD2/NM001018071.3 | PLEKHO1/NM016274.5 | ZNF460/NM006635.3 |
| FRRS1L/NM014334.2 | PLEKHO2/NM025201.4 | ZNF461/NM153257.4 |
| FRS2/NM001278357.1 | PLEKHS1/NM024889.4 | ZNF462/NM021224.4 |
| FRS3/NM006653.4 | PLET1/NM001145024.1 | ZNF467/NM207336.1 |
| FRY/NM023037.2 | PLGLB1/NM001032392.2 | ZNF468/NM001008801.1 |
| FRYL/NM015030.1 | PLGLB2/NM002665.4 | ZNF469/NM001127464.2 |
| FRZB/NM001463.3 | PLGRKT/NM018465.3 | ZNF470/NM001001668.3 |
| FSBP/NM001256141.1 | PLIN1/NM002666.4 | ZNF471/NM020813.3 |
| FSCB/NM032135.3 | PLIN2/NM001122.3 | ZNF473/NM001006656.2 |
| FSCN1/NM003088.3 | PLIN3/NM005817.4 | ZNF474/NM207317.2 |
| FSCN2/NM012418.3 | PLIN5/NM001013706.2 | ZNF479/NM033273.2 |
| FSCN3/NM020369.2 | PLK1/NM005030.5 | ZNF48/NM001214906.1 |
| FSD1/NM024333.2 | PLK3/NM004073.3 | ZNF48/NM001214909.1 |
| FSD1L/NM001287192.1 | PLK4/NM014264.4 | ZNF48/NM152652.2 |
| FSD2/NM001007122.3 | PLK5/NM001243079.1 | ZNF480/NM144684.3 |
| FSHB/NM001018080.1 | PLLP/NM015993.2 | ZNF483/NM133464.3 |
| FSIP1/NM152597.4 | PLN/NM002667.4 | ZNF484/NM031486.2 |
| FSIP2/NM173651.3 | PLOD1/NM000302.3 | ZNF485/NM001318141.1 |
| FST/NM013409.2 | PLOD2/NM182943.2 | ZNF486/NM052852.3 |
| FSTL1/NM007085.4 | PLOD3/NM001084.4 | ZNF488/NM153034.2 |
| FSTL3/NM005860.2 | PLPP1/NM176895.2 | ZNF490/NM020714.2 |
| FSTL4/NM015082.1 | PLPP2/NM003712.3 | ZNF491/NM152356.3 |
| FTCD/NM006657.2 | PLPP3/NM003713.4 | ZNF492/NM020855.2 |
| FTH1/NM002032.2 | PLPP4/NM001030059.2 | ZNF493/NM001076678.2 |
| FTL/NM000146.3 | PLPP5/NM001102559.1 | ZNF496/NM032752.1 |
| FTMT/NM177478.1 | PLPP6/NM203453.3 | ZNF497/NM198458.2 |
| FTO/NM001080432.2 | PLPP7/NM032728.3 | ZNF500/NM021646.3 |
| FTSJ3/NM017647.3 | PLPPR1/NM017753.2 | ZNF501/NM145044.3 |
| FUBP1/NM003902.4 | PLPPR2/NM022737.2 | ZNF502/NM001134442.1 |
| FUBP3/NM003934.1 | PLPPR3/NM024888.2 | ZNF503/NM032772.5 |
| FUCA1/NM000147.4 | PLPPR4/NM014839.4 | ZNF507/NM014910.4 |
| FUCA2/NM032020.4 | PLPPR5/NM001037317.1 | ZNF510/NM014930.2 |
| FUOM/NM001098483.2 | PLRG1/NM002669.3 | ZNF511/NM145806.3 |
| FURIN/NM001289823.1 | PLS1/NM001145319.1 | ZNF512/NM032434.3 |
| FURIN/NM001289824.1 | PLS1/NM001172312.1 | ZNF512B/NM020713.2 |
| FURIN/NM002569.3 | PLS1/NM002670.2 | ZNF513/NM144631.5 |
| FUS/NM004960.3 | PLSCR1/NM021105.2 | ZNF514/NM032788.2 |
| FUT1/NM000148.3 | PLSCR2/NM020359.2 | ZNF516/NM014643.3 |
| FUT10/NM032664.3 | PLSCR3/NM020360.3 | ZNF517/NM213605.2 |
| FUT11/NM173540.2 | PLSCR4/NM001128304.1 | ZNF518A/NM001278524.1 |
| FUT2/NM001097638.2 | PLSCR4/NM001128305.1 | ZNF518A/NM001278525.1 |
| FUT3/NM000149.3 | PLSCR5/NM001085420.1 | ZNF518B/NM053042.2 |
| FUT4/NM002033.3 | PLTP/NM006227.3 | ZNF519/NM145287.3 |
| FUT5/NM002034.2 | PLVAP/NM031310.2 | ZNF521/NM015461.2 |
| FUT6/NM000150.2 | PLXDC1/NM020405.4 | ZNF524/NM153219.3 |
| FUT7/NM004479.3 | PLXDC2/NM032812.8 | ZNF526/NM133444.2 |
| FUT8/NM178156.2 | PLXNA1/NM032242.3 | ZNF528/NM032423.2 |
| FUT9/NM006581.3 | PLXNA2/NM025179.3 | ZNF529/NM001145649.1 |
| FUZ/NM025129.4 | PLXNA4/NM020911.1 | ZNF530/NM020880.4 |
| FXN/NM000144.4 | PLXNB1/NM001130082.2 | ZNF532/NM018181.5 |
| FXR2/NM004860.3 | PLXNB1/NM002673.5 | ZNF534/NM001143939.1 |
| FXYD1/NM001278717.1 | PLXNB2/NM012401.3 | ZNF536/NM014717.2 |
| FXYD1/NM001278718.1 | PLXNC1/NM005761.2 | ZNF540/NM001172225.2 |
| FXYD1/NM005031.4 | PLXND1/NM015103.2 | ZNF540/NM152606.4 |
| FXYD1/NM021902.3 | PM20D1/NM152491.4 | ZNF541/NM001277075.1 |
| FXYD2/NM001680.4 | PM20D2/NM001010853.2 | ZNF543/NM213598.3 |
| FXYD3/NM005971.3 | PMAIP1/NM021127.2 | ZNF544/NM014480.3 |
| FXYD4/NM173160.2 | PMCH/NM002674.3 | ZNF546/NM178544.4 |
| FXYD5/NM001164605.1 | PMEL/NM006928.4 | ZNF547/NM173631.3 |
| FXYD5/NM014164.5 | PMEPA1/NM020182.4 | ZNF548/NM152909.3 |
| FXYD5/NM144779.2 | PMF1/NM007221.3 | ZNF549/NM153263.2 |
| FXYD6-FXYD2/NM001204268.1 | PMFBP1/NM031293.2 | ZNF550/NM001277090.1 |
| FXYD6/NM001164831.2 | PML/NM033238.2 | ZNF552/NM024762.3 |
| FXYD7/NM022006.1 | PMM1/NM002676.2 | ZNF554/NM001102651.1 |
| FYB/NM199335.3 | PMM2/NM000303.2 | ZNF555/NM152791.4 |
| FYCO1/NM024513.3 | PMP22/NM000304.3 | ZNF556/NM024967.2 |
| FYN/NM002037.5 | PMP22/NM001281455.1 | ZNF557/NM001044387.1 |
| FYTTD1/NM032288.6 | PMP22/NM153322.2 | ZNF558/NM144693.2 |
| FZD1/NM003505.1 | PMPCA/NM015160.2 | ZNF560/NM152476.2 |
| FZD10/NM007197.3 | PMPCB/NM004279.2 | ZNF561/NM152289.2 |
| FZD2/NM001466.3 | PMS1/NM000534.4 | ZNF562/NM001130031.1 |
| FZD3/NM145866.1 | PMS2/NM000535.6 | ZNF563/NM145276.2 |
| FZD4/NM012193.3 | PMVK/NM006556.3 | ZNF564/NM144976.3 |
| FZD5/NM003468.3 | PNISR/NM001322410.1 | ZNF565/NM001042474.2 |
| FZD6/NM001164615.1 | PNKD/NM015488.4 | ZNF565/NM152477.4 |
| FZD6/NM003506.3 | PNKP/NM007254.3 | ZNF566/NM001145345.1 |
| FZD7/NM003507.1 | PNLDC1/NM001271862.1 | ZNF566/NM032838.4 |
| FZD8/NM031866.2 | PNLIPRP2/NM005396.4 | ZNF567/NM001300979.1 |
| FZD9/NM003508.2 | PNLIPRP3/NM001011709.2 | ZNF568/NM198539.3 |
| FZR1/NM016263.3 | PNMA1/NM006029.4 | ZNF569/NM152484.2 |
| G0S2/NM015714.3 | PNMA2/NM007257.5 | ZNF57/NM173480.2 |
| G2E3/NM017769.4 | PNMAL1/NM018215.3 | ZNF570/NM001321991.1 |
| G3BP1/NM198395.1 | PNMAL2/NM020709.2 | ZNF570/NM144694.3 |
| G3BP2/NM012297.4 | PNMT/NM002686.4 | ZNF571/NM001290314.1 |
| G3BP2/NM203505.2 | PNN/NM002687.3 | ZNF572/NM152412.2 |
| G6PC2/NM021176.2 | PNOC/NM006228.4 | ZNF573/NM001172690.1 |
| G6PC3/NM138387.3 | PNP/NM000270.3 | ZNF574/NM022752.5 |
| GAA/NM001079804.2 | PNPLA1/NM001145717.1 | ZNF575/NM174945.2 |
| GAB1/NM207123.2 | PNPLA2/NM020376.3 | ZNF576/NM001145347.1 |
| GAB2/NM080491.2 | PNPLA3/NM025225.2 | ZNF576/NM024327.2 |
| GAB4/NM001037814.1 | PNPLA5/NM138814.3 | ZNF577/NM032679.2 |
| GABARAP/NM007278.1 | PNPLA6/NM001166113.1 | ZNF578/NM001099694.1 |
| GABARAPL1/NM031412.2 | PNPLA6/NM006702.4 | ZNF579/NM152600.2 |
| GABARAPL2/NM007285.6 | PNPLA7/NM152286.4 | ZNF580/NM001163423.1 |
| GABBR1/NM001470.3 | PNPLA8/NM015723.4 | ZNF580/NM016202.2 |
| GABBR2/NM005458.7 | PNPO/NM018129.3 | ZNF580/NM207115.1 |
| GABPB1/NM016654.4 | PNPT1/NM033109.4 | ZNF581/NM016535.3 |
| GABPB2/NM144618.2 | PNRC1/NM006813.2 | ZNF582/NM144690.2 |
| GABRA1/NM001127648.1 | PNRC2/NM017761.3 | ZNF583/NM001159860.1 |
| GABRA2/NM000807.2 | POC1A/NM015426.4 | ZNF583/NM001159861.1 |
| GABRA2/NM001114175.1 | POC1B/NM172240.2 | ZNF583/NM152478.2 |
| GABRA6/NM000811.2 | POC5/NM001099271.1 | ZNF584/NM173548.2 |
| GABRB1/NM000812.3 | PODN/NM153703.4 | ZNF585A/NM001288800.1 |
| GABRB3/NM000814.5 | PODXL/NM001018111.2 | ZNF585B/NM152279.3 |
| GABRD/NM000815.4 | PODXL2/NM015720.3 | ZNF586/NM017652.3 |
| GABRG1/NM173536.3 | POFUT1/NM015352.1 | ZNF587/NM032828.3 |
| GABRG2/NM198904.2 | POFUT2/NM133635.4 | ZNF587B/NM001204818.1 |
| GABRR1/NM002042.4 | POGK/NM017542.4 | ZNF589/NM016089.2 |
| GABRR2/NM002043.4 | POGLUT1/NM152305.2 | ZNF592/NM014630.2 |
| GABRR3/NM001105580.2 | POGZ/NM015100.3 | ZNF593/NM015871.4 |
| GAD1/NM000817.2 | POLA2/NM002689.3 | ZNF594/NM032530.1 |
| GADD45A/NM001924.3 | POLD1/NM002691.3 | ZNF595/NM182524.3 |
| GADD45B/NM015675.3 | POLD2/NM001127218.2 | ZNF596/NM001042415.2 |
| GADD45G/NM006705.3 | POLD2/NM001256879.1 | ZNF596/NM001042416.2 |
| GADD45GIP1/NM052850.3 | POLD3/NM006591.2 | ZNF596/NM001287254.1 |
| GAK/NM005255.3 | POLD4/NM021173.4 | ZNF596/NM001287256.1 |
| GAL/NM015973.4 | POLDIP2/NM015584.4 | ZNF596/NM173539.3 |
| GAL3ST1/NM004861.2 | POLE/NM006231.3 | ZNF597/NM152457.2 |
| GAL3ST2/NM022134.2 | POLE2/NM002692.3 | ZNF598/NM178167.3 |
| GAL3ST3/NM033036.2 | POLE3/NM017443.4 | ZNF599/NM001007248.2 |
| GAL3ST4/NM024637.4 | POLE4/NM019896.2 | ZNF600/NM001321866.1 |
| GALC/NM000153.3 | POLG/NM002693.2 | ZNF605/NM183238.3 |
| GALE/NM000403.3 | POLG2/NM007215.3 | ZNF606/NM025027.3 |
| GALE/NM001008216.1 | POLH/NM006502.2 | ZNF607/NM032689.4 |
| GALE/NM001127621.1 | POLI/NM007195.2 | ZNF608/NM020747.2 |
| GALK1/NM000154.1 | POLK/NM016218.2 | ZNF609/NM015042.1 |
| GALK2/NM001001556.2 | POLL/NM001174084.1 | ZNF610/NM001161426.1 |
| GALNS/NM000512.4 | POLM/NM013284.3 | ZNF610/NM173530.2 |
| GALNT1/NM020474.3 | POLN/NM181808.3 | ZNF611/NM001161499.1 |
| GALNT10/NM198321.3 | POLQ/NM199420.3 | ZNF611/NM030972.3 |
| GALNT11/NM022087.3 | POLR1A/NM015425.3 | ZNF613/NM001031721.3 |
| GALNT12/NM024642.4 | POLR1B/NM019014.5 | ZNF614/NM025040.3 |
| GALNT14/NM024572.3 | POLR1C/NM203290.3 | ZNF615/NM198480.3 |
| GALNT15/NM054110.4 | POLR1D/NM015972.3 | ZNF616/NM178523.4 |
| GALNT18/NM198516.2 | POLR1E/NM022490.2 | ZNF618/NM001318042.1 |
| GALNT2/NM004481.4 | POLR2A/NM000937.4 | ZNF619/NM001145082.2 |
| GALNT3/NM004482.3 | POLR2B/NM000938.2 | ZNF620/NM175888.3 |
| GALNT4/NM003774.4 | POLR2D/NM004805.3 | ZNF621/NM001098414.2 |
| GALNT5/NM014568.1 | POLR2E/NM002695.3 | ZNF622/NM033414.2 |
| GALNT6/NM007210.3 | POLR2F/NM021974.4 | ZNF623/NM014789.3 |
| GALNT7/NM017423.2 | POLR2G/NM002696.2 | ZNF624/NM020787.3 |
| GALNT8/NM017417.1 | POLR2H/NM006232.3 | ZNF625/NM145233.3 |
| GALNT9/NM001122636.1 | POLR2I/NM006233.4 | ZNF626/NM001076675.2 |
| GALNTL6/NM001034845.2 | POLR2J/NM006234.4 | ZNF627/NM145295.3 |
| GALP/NM033106.3 | POLR2J2/NM032959.5 | ZNF628/NM033113.2 |
| GALR1/NM001480.3 | POLR2J3/NM001097615.2 | ZNF629/NM001080417.1 |
| GALR2/NM003857.3 | POLR2K/NM005034.3 | ZNF638/NM001014972.2 |
| GALR3/NM003614.1 | POLR2L/NM021128.4 | ZNF639/NM001303426.1 |
| GALT/NM000155.3 | POLR2M/NM015532.4 | ZNF639/NM016331.2 |
| GAMT/NM000156.5 | POLR3A/NM007055.3 | ZNF641/NM001172681.1 |
| GAN/NM022041.3 | POLR3B/NM018082.5 | ZNF644/NM201269.2 |
| GANAB/NM198334.2 | POLR3C/NM006468.7 | ZNF648/NM001009992.1 |
| GAP43/NM002045.3 | POLR3D/NM001722.2 | ZNF649/NM023074.3 |
| GAPDH/NM001289746.1 | POLR3E/NM018119.3 | ZNF652/NM001145365.1 |
| GAPDH/NM002046.5 | POLR3F/NM006466.3 | ZNF652/NM014897.2 |
| GAPDHS/NM014364.4 | POLR3G/NM006467.2 | ZNF653/NM138783.3 |
| GAPT/NM001304431.1 | POLR3GL/NM032305.1 | ZNF658/NM033160.6 |
| GAPVD1/NM001282680.1 | POLR3H/NM001018050.3 | ZNF660/NM173658.2 |
| GAR1/NM032993.2 | POLR3H/NM001282884.1 | ZNF662/NM001134656.1 |
| GAREM1/NM022751.2 | POLR3K/NM016310.4 | ZNF664/NM001204298.1 |
| GAREM2/NM001168241.1 | POLRMT/NM005035.3 | ZNF665/NM024733.3 |
| GARNL3/NM001286779.1 | POM121/NM172020.4 | ZNF667/NM001321356.1 |
| GARS/NM002047.3 | POM121C/NM001099415.2 | ZNF668/NM001172670.1 |
| GART/NM000819.4 | POM121L12/NM182595.3 | ZNF669/NM024804.2 |
| GART/NM001136005.1 | POM121L2/NM033482.3 | ZNF670/NM033213.4 |
| GART/NM001136006.1 | POMC/NM001319204.1 | ZNF671/NM024833.2 |
| GAS1/NM002048.2 | POMGNT2/NM032806.5 | ZNF672/NM024836.2 |
| GAS2/NM001143830.1 | POMK/NM001277971.1 | ZNF676/NM001001411.2 |
| GAS2/NM005256.3 | POMP/NM015932.5 | ZNF677/NM001317998.1 |
| GAS2/NM177553.2 | POMT1/NM001077365.1 | ZNF677/NM182609.3 |
| GAS2L1/NM006478.4 | POMT2/NM013382.5 | ZNF679/NM153363.2 |
| GAS2L2/NM139285.3 | POMZP3/NM012230.3 | ZNF680/NM178558.4 |
| GAS2L3/NM001303130.1 | PON1/NM000446.5 | ZNF681/NM138286.2 |
| GAS6/NM000820.3 | PON2/NM000305.2 | ZNF682/NM033196.2 |
| GAS7/NM201433.1 | PON3/NM000940.2 | ZNF683/NM001114759.2 |
| GAS8/NM001481.2 | POP1/NM001145861.1 | ZNF684/NM152373.3 |
| GAST/NM000805.4 | POP1/NM015029.2 | ZNF687/NM001304764.1 |
| GATA2/NM001145661.1 | POP3/NM001320010.1 | ZNF687/NM020832.2 |
| GATA2/NM032638.4 | POP4/NM006627.2 | ZNF688/NM145271.3 |
| GATA3/NM002051.2 | POP5/NM015918.3 | ZNF689/NM138447.2 |
| GATA5/NM080473.4 | POP7/NM005837.2 | ZNF691/NM015911.3 |
| GATA6/NM005257.5 | POPDC2/NM022135.3 | ZNF692/NM017865.3 |
| GATAD1/NM021167.4 | POPDC3/NM022361.4 | ZNF695/NM020394.4 |
| GATAD2A/NM017660.3 | POR/NM000941.2 | ZNF696/NM030895.2 |
| GATAD2B/NM020699.2 | POSTN/NM006475.2 | ZNF697/NM001080470.1 |
| GATB/NM004564.2 | POT1/NM015450.2 | ZNF699/NM198535.2 |
| GATC/NM176818.2 | POTEA/NM001005365.2 | ZNF7/NM003416.3 |
| GATM/NM001482.2 | POTEB/NM001277304.1 | ZNF70/NM021916.3 |
| GATS/NM178831.6 | POTEB2/NM001277303.1 | ZNF700/NM144566.2 |
| GATSL2/NM001145064.2 | POTEB3/NM207355.3 | ZNF701/NM018260.2 |
| GATSL3/NM001037666.2 | POTED/NM174981.3 | ZNF703/NM025069.2 |
| GBA/NM000157.3 | POTEE/NM001083538.1 | ZNF704/NM001033723.2 |
| GBA/NM001005741.2 | POTEF/NM001099771.2 | ZNF705A/NM001004328.2 |
| GBA2/NM020944.2 | POTEG/NM001005356.2 | ZNF705B/NM001193630.1 |
| GBAS/NM001483.2 | POTEH/NM001136213.1 | ZNF705D/NM001039615.3 |
| GBE1/NM000158.3 | POTEI/NM001277406.1 | ZNF705E/NM001278713.1 |
| GBF1/NM004193.2 | POTEJ/NM001277083.1 | ZNF705G/NM001164457.1 |
| GBGT1/NM021996.5 | POTEM/NM001145442.1 | ZNF706/NM001267708.1 |
| GBP1/NM002053.2 | POU1F1/NM000306.3 | ZNF706/NM001267709.1 |
| GBP2/NM004120.4 | POU2AF1/NM006235.2 | ZNF708/NM021269.2 |
| GBP3/NM018284.2 | POU2F1/NM002697.3 | ZNF709/NM152601.3 |
| GBP4/NM052941.4 | POU2F2/NM002698.4 | ZNF713/NM182633.2 |
| GBP5/NM052942.3 | POU2F3/NM014352.3 | ZNF714/NM182515.3 |
| GBP6/NM198460.2 | POU3F1/NM002699.3 | ZNF716/NM001159279.1 |
| GBP7/NM207398.2 | POU3F2/NM005604.3 | ZNF717/NM001128223.2 |
| GBX1/NM001098834.2 | POU3F3/NM006236.2 | ZNF717/NM001290208.1 |
| GC/NM000583.3 | POU4F1/NM006237.3 | ZNF718/NM001039127.5 |
| GC/NM001204306.1 | POU4F2/NM004575.2 | ZNF720/NM001130913.1 |
| GCA/NM012198.3 | POU4F3/NM002700.2 | ZNF721/NM133474.3 |
| GCAT/NM014291.3 | POU5F1/NM002701.5 | ZNF726/NM001244038.1 |
| GCC1/NM024523.5 | POU5F1B/NM001159542.1 | ZNF727/NM001159522.1 |
| GCC2/NM181453.3 | POU5F2/NM153216.1 | ZNF728/NM001267716.1 |
| GCDH/NM000159.3 | POU6F1/NM002702.3 | ZNF729/NM001242680.1 |
| GCFC2/NM003203.4 | POU6F2/NM001166018.1 | ZNF730/NM001277403.1 |
| GCG/NM002054.4 | PP2D1/NM001252657.1 | ZNF732/NM001137608.1 |
| GCGR/NM000160.4 | PPA1/NM021129.3 | ZNF735/NM001159524.1 |
| GCH1/NM000161.2 | PPA2/NM176869.2 | ZNF736/NM001170905.2 |
| GCHFR/NM005258.2 | PPAN/NM020230.5 | ZNF737/NM001159293.1 |
| GCK/NM000162.3 | PPARA/NM001001928.2 | ZNF74/NM001256524.1 |
| GCLC/NM001498.3 | PPARD/NM006238.4 | ZNF740/NM001004304.3 |
| GCLM/NM002061.3 | PPARG/NM015869.4 | ZNF747/NM001305019.1 |
| GCM1/NM003643.3 | PPARGC1A/NM013261.3 | ZNF749/NM001023561.3 |
| GCM2/NM004752.3 | PPARGC1B/NM133263.3 | ZNF750/NM024702.2 |
| GCN1/NM006836.1 | PPAT/NM002703.4 | ZNF76/NM003427.4 |
| GCNT1/NM001097633.1 | PPBP/NM002704.3 | ZNF761/NM001289951.1 |
| GCNT1/NM001097634.1 | PPCDC/NM021823.4 | ZNF761/NM001289952.1 |
| GCNT1/NM001097635.1 | PPCS/NM024664.3 | ZNF763/NM001012753.1 |
| GCNT1/NM001097636.1 | PPDPF/NM024299.2 | ZNF764/NM033410.3 |
| GCNT2/NM145655.3 | PPEF2/NM006239.2 | ZNF766/NM001010851.2 |
| GCNT3/NM004751.2 | PPFIA2/NM003625.4 | ZNF768/NM024671.3 |
| GCNT4/NM016591.2 | PPFIA3/NM003660.3 | ZNF770/NM014106.3 |
| GCNT7/NM080615.1 | PPFIA4/NM001304331.1 | ZNF771/NM001142305.1 |
| GCSAML/NM001281853.1 | PPFIBP1/NM003622.3 | ZNF772/NM001024596.2 |
| GCSAML/NM145278.4 | PPFIBP2/NM003621.3 | ZNF773/NM198542.2 |
| GCSH/NM004483.4 | PPHLN1/NM201439.1 | ZNF774/NM001004309.2 |
| GDA/NM004293.4 | PPIA/NM021130.4 | ZNF775/NM173680.3 |
| GDAP1/NM018972.2 | PPIAL4A/NM001143883.2 | ZNF777/NM015694.2 |
| GDAP1L1/NM024034.4 | PPIAL4C/NM001135789.3 | ZNF778/NM182531.3 |
| GDAP2/NM017686.3 | PPIAL4D/NM001164261.1 | ZNF780A/NM001010880.2 |
| GDE1/NM016641.3 | PPIAL4E/NM001144032.2 | ZNF780A/NM001142578.1 |
| GDF1/NM001492.5 | PPIAL4F/NM001164262.2 | ZNF780B/NM001005851.2 |
| GDF10/NM004962.3 | PPIAL4G/NM001123068.1 | ZNF781/NM152605.3 |
| GDF11/NM005811.3 | PPIC/NM000943.4 | ZNF782/NM001001662.1 |
| GDF15/NM004864.2 | PPID/NM005038.2 | ZNF783/NM001195220.1 |
| GDF2/NM016204.2 | PPIE/NM006112.3 | ZNF784/NM203374.1 |
| GDF3/NM020634.1 | PPIF/NM005729.3 | ZNF785/NM152458.6 |
| GDF5/NM001319138.1 | PPIG/NM004792.2 | ZNF786/NM152411.3 |
| GDF6/NM001001557.2 | PPIH/NM006347.3 | ZNF787/NM001002836.3 |
| GDF7/NM182828.2 | PPIL1/NM016059.4 | ZNF789/NM213603.2 |
| GDF9/NM005260.4 | PPIL2/NM148175.2 | ZNF79/NM007135.2 |
| GDI2/NM001494.3 | PPIL3/NM130906.2 | ZNF790/NM001242800.1 |
| GDNF-AS1/NM001278094.1 | PPIL4/NM139126.3 | ZNF790/NM001242801.1 |
| GDNF/NM000514.3 | PPIP5K1/NM001130859.2 | ZNF790/NM001242802.1 |
| GDPD1/NM182569.3 | PPIP5K1/NM014659.5 | ZNF790/NM206894.3 |
| GDPD3/NM024307.2 | PPIP5K2/NM015216.3 | ZNF791/NM153358.2 |
| GDPD4/NM182833.1 | PPL/NM002705.4 | ZNF792/NM175872.4 |
| GDPD5/NM030792.6 | PPM1A/NM021003.4 | ZNF793/NM001013659.2 |
| GDPGP1/NM001013657.2 | PPM1B/NM002706.5 | ZNF799/NM001080821.2 |
| GEM/NM005261.3 | PPM1E/NM014906.4 | ZNF8/NM021089.2 |
| GEMIN2/NM003616.2 | PPM1F/NM014634.3 | ZNF80/NM007136.3 |
| GEMIN4/NM015721.2 | PPM1H/NM020700.1 | ZNF800/NM176814.4 |
| GEMIN5/NM015465.4 | PPM1J/NM005167.5 | ZNF804A/NM194250.1 |
| GEMIN6/NM024775.9 | PPM1K/NM152542.4 | ZNF804B/NM181646.3 |
| GEMIN7/NM001007269.1 | PPM1M/NM144641.3 | ZNF805/NM001023563.3 |
| GEMIN7/NM001007270.1 | PPM1N/NM001080401.1 | ZNF806/NM001304449.1 |
| GEMIN7/NM001319054.1 | PPME1/NM016147.2 | ZNF813/NM001004301.3 |
| GEMIN7/NM001319055.1 | PPOX/NM000309.3 | ZNF814/NM001144989.1 |
| GEN1/NM001130009.2 | PPP1CB/NM002709.2 | ZNF816-ZNF321P/NM001202473.1 |
| GEN1/NM182625.4 | PPP1CB/NM206876.1 | ZNF821/NM001201554.1 |
| GET4/NM015949.2 | PPP1CC/NM002710.3 | ZNF821/NM017530.2 |
| GFER/NM005262.2 | PPP1R10/NM002714.3 | ZNF823/NM001080493.3 |
| GFI1/NM001127215.1 | PPP1R11/NM021959.2 | ZNF829/NM001037232.3 |
| GFI1/NM001127216.1 | PPP1R12A/NM001143885.1 | ZNF83/NM001277947.1 |
| GFI1/NM005263.3 | PPP1R12A/NM002480.2 | ZNF83/NM001277951.1 |
| GFI1B/NM004188.6 | PPP1R12B/NM002481.3 | ZNF830/NM052857.3 |
| GFM1/NM024996.5 | PPP1R12C/NM017607.3 | ZNF831/NM178457.2 |
| GFM2/NM032380.4 | PPP1R13B/NM015316.2 | ZNF835/NM001005850.2 |
| GFOD1/NM018988.3 | PPP1R13L/NM001142502.1 | ZNF836/NM001102657.1 |
| GFOD2/NM030819.3 | PPP1R13L/NM006663.3 | ZNF837/NM138466.1 |
| GFPT1/NM002056.3 | PPP1R14A/NM033256.2 | ZNF839/NM018335.4 |
| GFPT2/NM005110.3 | PPP1R14B/NM138689.2 | ZNF84/NM001289972.1 |
| GFRA1/NM001145453.2 | PPP1R14C/NM030949.2 | ZNF84/NM003428.5 |
| GFRA1/NM145793.3 | PPP1R14D/NM017726.7 | ZNF841/NM001136499.1 |
| GFRA2/NM001495.4 | PPP1R15B/NM032833.4 | ZNF843/NM001136509.1 |
| GFRA4/NM145762.2 | PPP1R16A/NM032902.5 | ZNF845/NM138374.2 |
| GFRAL/NM207410.2 | PPP1R16B/NM015568.3 | ZNF846/NM001077624.1 |
| GFY/NM001195256.1 | PPP1R17/NM006658.4 | ZNF85/NM003429.4 |
| GGA1/NM013365.4 | PPP1R18/NM001134870.1 | ZNF850/NM001193552.1 |
| GGA2/NM015044.4 | PPP1R18/NM133471.3 | ZNF852/NM001287349.1 |
| GGA3/NM138619.3 | PPP1R1A/NM006741.3 | ZNF853/NM017560.1 |
| GGACT/NM001195087.1 | PPP1R1B/NM032192.3 | ZNF860/NM001137674.2 |
| GGACT/NM033110.2 | PPP1R2/NM006241.7 | ZNF865/NM001195605.1 |
| GGCT/NM024051.3 | PPP1R21/NM001135629.2 | ZNF878/NM001080404.2 |
| GGCX/NM000821.6 | PPP1R26/NM014811.3 | ZNF879/NM001136116.1 |
| GGH/NM003878.2 | PPP1R27/NM001007533.3 | ZNF880/NM001145434.1 |
| GGN/NM152657.3 | PPP1R32/NM145017.2 | ZNF883/NM001101338.1 |
| GGNBP2/NM024835.4 | PPP1R35/NM145030.2 | ZNF888/NM001310127.1 |
| GGT1/NM013421.2 | PPP1R36/NM172365.2 | ZNF891/NM001277291.1 |
| GGT1/NM013430.2 | PPP1R37/NM019121.1 | ZNF90/NM007138.1 |
| GGT5/NM004121.3 | PPP1R3B/NM001201329.1 | ZNF91/NM003430.3 |
| GGT6/NM001122890.2 | PPP1R3B/NM024607.3 | ZNF92/NM152626.3 |
| GGT7/NM178026.2 | PPP1R3C/NM005398.5 | ZNF93/NM031218.3 |
| GGTLC1/NM178311.2 | PPP1R3D/NM006242.3 | ZNF98/NM001098626.1 |
| GGTLC1/NM178312.2 | PPP1R3E/NM001276318.1 | ZNF99/NM001080409.2 |
| GGTLC2/NM001282879.1 | PPP1R3G/NM001145115.2 | ZNFX1/NM021035.2 |
| GH1/NM000515.4 | PPP1R42/NM001013626.2 | ZNHIT1/NM006349.2 |
| GH2/NM002059.4 | PPP1R8/NM014110.4 | ZNHIT2/NM014205.3 |
| GHDC/NM032484.4 | PPP1R9A/NM017650.2 | ZNHIT3/NM004773.3 |
| GHITM/NM014394.2 | PPP1R9B/NM032595.4 | ZNHIT6/NM017953.3 |
| GHR/NM000163.4 | PPP2CB/NM001009552.1 | ZNRD1/NM001278785.1 |
| GHR/NM001242400.2 | PPP2R1A/NM014225.5 | ZNRF1/NM032268.4 |
| GHR/NM001242401.3 | PPP2R1B/NM181699.2 | ZNRF2/NM147128.3 |
| GHR/NM001242402.2 | PPP2R2A/NM002717.3 | ZNRF3/NM032173.3 |
| GHR/NM001242403.2 | PPP2R2B/NM181676.2 | ZNRF4/NM181710.3 |
| GHR/NM001242404.2 | PPP2R2C/NM020416.3 | ZP1/NM207341.3 |
| GHR/NM001242405.2 | PPP2R2D/NM018461.4 | ZP2/NM003460.2 |
| GHR/NM001242406.2 | PPP2R3A/NM181897.2 | ZP3/NM007155.5 |
| GHRH/NM021081.5 | PPP2R3C/NM017917.3 | ZP4/NM021186.3 |
| GHRHR/NM000823.3 | PPP2R4/NM021131.4 | ZPBP/NM007009.2 |
| GHRL/NM016362.4 | PPP2R4/NM178000.2 | ZPR1/NM003904.4 |
| GHSR/NM198407.2 | PPP2R5A/NM006243.3 | ZRANB1/NM017580.2 |
| GID8/NM017896.2 | PPP2R5B/NM006244.3 | ZRANB2/NM203350.2 |
| GIF/NM005142.2 | PPP2R5C/NM002719.3 | ZRANB3/NM032143.3 |
| GIGYF1/NM022574.4 | PPP2R5D/NM006245.3 | ZSCAN1/NM182572.3 |
| GIGYF2/NM001103146.1 | PPP2R5E/NM006246.3 | ZSCAN10/NM032805.2 |
| GIMAP1-GIMAP5/NM001199577.1 | PPP3CA/NM000944.4 | ZSCAN12/NM001163391.1 |
| GIMAP4/NM018326.2 | PPP3CB/NM021132.3 | ZSCAN16/NM025231.2 |
| GIMAP5/NM018384.4 | PPP3CC/NM005605.4 | ZSCAN18/NM023926.4 |
| GIMAP6/NM024711.5 | PPP3R1/NM000945.3 | ZSCAN2/NM181877.3 |
| GIMAP7/NM153236.3 | PPP3R2/NM147180.3 | ZSCAN20/NM145238.3 |
| GIMAP8/NM175571.3 | PPP4C/NM002720.2 | ZSCAN21/NM145914.2 |
| GIMD1/NM001195138.1 | PPP4R1/NM001042388.2 | ZSCAN22/NM001321116.1 |
| GIN1/NM017676.2 | PPP4R2/NM174907.3 | ZSCAN23/NM001012455.1 |
| GINS1/NM021067.4 | PPP4R3A/NM001284280.1 | ZSCAN26/NM001111039.2 |
| GINS2/NM016095.2 | PPP4R3B/NM001282850.1 | ZSCAN29/NM152455.3 |
| GINS3/NM022770.3 | PPP4R4/NM058237.1 | ZSCAN30/NM001166012.2 |
| GIP/NM004123.2 | PPP5C/NM006247.3 | ZSCAN31/NM001135215.1 |
| GIPC1/NM005716.3 | PPP5D1/NM001205281.1 | ZSCAN31/NM001135216.1 |
| GIPC2/NM017655.5 | PPP6R1/NM014931.3 | ZSCAN31/NM030899.4 |
| GIPC3/NM133261.2 | PPP6R2/NM014678.4 | ZSCAN31/NM145909.2 |
| GIPR/NM000164.3 | PPP6R3/NM001164160.1 | ZSCAN4/NM152677.2 |
| GIT1/NM014030.3 | PPT1/NM000310.3 | ZSCAN5A/NM024303.2 |
| GIT2/NM057169.3 | PPT2/NM138717.2 | ZSCAN5B/NM001080456.2 |
| GJA1/NM000165.4 | PPTC7/NM139283.1 | ZSCAN9/NM001199480.1 |
| GJA10/NM032602.1 | PPY/NM002722.4 | ZSWIM1/NM080603.4 |
| GJA3/NM021954.3 | PQLC1/NM025078.4 | ZSWIM4/NM023072.2 |
| GJA4/NM002060.2 | PQLC2/NM017765.2 | ZSWIM5/NM020883.1 |
| GJA5/NM005266.6 | PQLC3/NM152391.4 | ZSWIM6/NM020928.1 |
| GJA5/NM181703.3 | PRAC1/NM032391.2 | ZSWIM7/NM001042697.1 |
| GJA8/NM005267.4 | PRAC2/NM001282276.1 | ZSWIM8/NM015037.3 |
| GJA9/NM030772.4 | PRADC1/NM032319.1 | ZUFSP/NM145062.2 |
| GJB2/NM004004.5 | PRAM1/NM032152.4 | ZW10/NM004724.3 |
| GJB3/NM001005752.1 | PRAMEF1/NM023013.4 | ZWILCH/NM017975.4 |
| GJB3/NM024009.2 | PRAMEF10/NM001039361.4 | ZWINT/NM032997.2 |
| GJB4/NM153212.2 | PRAMEF11/NM001146344.2 | ZXDC/NM025112.4 |
| GJB5/NM005268.3 | PRAMEF12/NM001080830.4 | ZYG11A/NM001004339.2 |
| GJB6/NM001110219.2 | PRAMEF13/NM001291380.1 | ZYG11B/NM024646.2 |
| GJB6/NM006783.4 | PRAMEF14/NM001024661.2 | ZYX/NM001010972.1 |
| GJB7/NM198568.2 | PRAMEF15/NM001098376.2 | ZZEF1/NM015113.3 |
| GJC1/NM001080383.1 | PRAMEF17/NM001099851.3 | ZZZ3/NM015534.5 |
| GJC1/NM005497.3 | PRAMEF18/NM001099850.2 | |
| GJC2/NM020435.3 | PRAMEF19/NM001099790.2 | |
| GJC3/NM181538.2 | PRAMEF2/NM023014.1 | |
| GJD2/NM020660.2 | PRAMEF20/NM001099852.2 | |
| GJD3/NM152219.3 | PRAMEF25/NM001310134.1 | |
| GJD4/NM153368.2 | PRAMEF26/NM001306072.1 | |
| GK2/NM033214.2 | PRAMEF27/NM001300891.1 | |
| GK5/NM001039547.2 | PRAMEF33P/NM001291381.1 | |
| GKAP1/NM025211.3 | PRAMEF4/NM001009611.4 | |
| GLB1/NM000404.3 | PRAMEF5/NM001013407.2 | |
| GLB1L/NM001286423.1 | PRAMEF6/NM001010889.2 | |
| GLB1L2/NM138342.3 | PRAMEF7/NM001012277.4 | |
| GLB1L3/NM001080407.2 | PRAMEF8/NM001012276.3 | |
| GLCCI1/NM138426.3 | PRAMEF9/NM001010890.2 | |
| GLCE/NM015554.2 | PRAP1/NM145202.4 | |
| GLDC/NM000170.2 | PRB1/NM005039.3 | |
| GLDN/NM181789.2 | PRB2/NM006248.3 | |
| GLE1/NM001003722.1 | PRB3/NM006249.5 | |
| GLG1/NM012201.5 | PRB4/NM002723.5 | |
| GLI1/NM005269.2 | PRC1/NM003981.3 | |
| GLI2/NM005270.4 | PRCC/NM005973.4 | |
| GLI3/NM000168.5 | PRCD/NM001077620.2 | |
| GLI4/NM138465.3 | PRCP/NM005040.3 | |
| GLIPR1L1/NM152779.3 | PRDM1/NM001198.3 | |
| GLIS1/NM147193.2 | PRDM10/NM199437.1 | |
| GLIS2/NM001318918.1 | PRDM11/NM001256695.1 | |
| GLIS2/NM032575.2 | PRDM12/NM021619.2 | |
| GLIS3/NM152629.3 | PRDM13/NM021620.3 | |
| GLMN/NM053274.2 | PRDM14/NM024504.3 | |
| GLMP/NM144580.2 | PRDM15/NM022115.4 | |

In an aspect, the methods and systems described herein for inferring gene expression may comprise detecting and/or staging a disease in a subject. The detection and/or staging of the disease may be based, at least in part, on the gene expression score of one or more genes from a plurality of genes of a subject.

In some embodiments, the methods may comprise preparing a methylation sequencing library for inferring gene expression. The methods may comprise obtaining a biological sample from a subject. The methods may comprise extracting cell-free deoxyribonucleic acid (cfDNA) from the biological sample, wherein the cfDNA comprises a plurality of cfDNA fragments. The methods may comprise providing conditions capable of converting unmethylated cytosines to uracils in the cfDNA fragments to produce a plurality of converted cfDNA fragments. The methods may comprise enriching the plurality of converted cfDNA fragments to produce enriched converted cfDNA fragment molecule. The enriching may comprise contacting the plurality of converted cfDNA fragments with a probe set comprising hybridization probes having sequence complementarity to at least two transcription start site (TSS) sequences of a plurality of TSS sequences selected from the genes listed in Tables 1-5. The methods may comprise amplifying the enriched converted cfDNA fragment molecules to produce amplified enriched converted cfDNA fragments. The methods may comprise determining a nucleic acid sequence of a plurality of cfDNA sequencing fragments of the amplified enriched converted cfDNA fragments. The methods may comprise processing the plurality of cfDNA sequencing fragments. The processing may comprise calculating a gene expression score for one or more genes of a plurality of genes. The gene expression score may indicate a probability of expression or non-expression of the one or more genes of the plurality of genes. The methods may comprise detecting a presence or an absence of a disease in the subject based on the determining a nucleic acid sequence of a plurality of cfDNA sequencing fragments. The methods may comprise detecting a presence or an absence of a disease in the subject based on the processing the plurality of cfDNA sequencing fragments. The methods may comprise detecting a presence or an absence of a disease in the subject based on the calculated gene expression score for one or more genes of a plurality of genes.

In some embodiment, the methods may comprise preparing a sequencing library for inferring gene expression. The methods may comprise obtaining a biological sample from a subject. The methods may comprise extracting cell-free deoxyribonucleic acid (cfDNA) from the biological sample, wherein the cfDNA comprises a plurality of cfDNA fragments. The methods may comprise amplifying the plurality of cfDNA fragments to produce amplified cfDNA fragments. The methods may comprise determining a nucleic acid sequence of a plurality of cfDNA sequencing fragments of the amplified cfDNA fragments. The methods may comprise processing the plurality of sequenced cfDNA fragments. The processing may comprise calculating a gene expression score for one or more genes in a plurality of genes. The gene expression score may indicate a probability of expression or non-expression of the one or more genes of the plurality of genes. The methods may comprise detecting a presence or an absence of a disease in the subject based on the processing the plurality of sequenced cfDNA fragments. The methods may comprise detecting a presence or absence of a disease in the subject based on the gene expression score of the one or more genes.

The disease may comprise cancer. The cancer may comprise a combination of cancers or a combination of cancer types. In some embodiments, the cancer may comprise breast cancer, diffuse large B cell cancer, lymphoma, liver cancer, ovarian cancer, lung cancer, renal cancer, bladder cancer, prostate cancer, pancreatic cancer, cervical cancer, color cancer, testicular cancer, thyroid cancer, bile duct cancer, esophageal cancer, skin cancer, kidney cancer, endometrial cancer, small intestine cancer, or stomach cancer. The cancer may comprise a stage of a cancer. For example, the cancer may be stage I cancer, stage II cancer, stage III cancer, or stage IV cancer. The cancer may be an early-stage cancer (e.g., stage 0, I or II). The cancer may be a late-stage cancer (e.g., stage III or IV).

In some embodiments, the methods may detect a disease in a subject with an accuracy of at least 50%, at least 55%, at least 60%, at least 65%, at least 70%, at least 75%, at least 80%, at least 85%, at least 90%, at least 95%, at least 96%, at least 97%, at least 98%, or at least 99%.

In some embodiments, the methods may detect a disease in a subject with a sensitivity of at least 50%, at least 55%, at least 60%, at least 65%, at least 70%, at least 75%, at least 80%, at least 85%, at least 90%, at least 95%, at least 96%, at least 97%, at least 98%, or at least 99%.

In some embodiments, the methods may detect a disease in a subject with a specificity of at least 50%, at least 55%, at least 60%, at least 65%, at least 70%, at least 75%, at least 80%, at least 85%, at least 90%, at least 95%, at least 96%, at least 97%, at least 98%, or at least 99%.

In some embodiments, the methods may detect a disease in a subject with an Area Under the Curve (AUC) value of a Receiver Operating Characteristic (ROC) curve of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or least about 0.99.

In some embodiments, the methods may detect a disease in a subject with a negative predictive value (NPV) of at least 50%, at least 55%, at least 60%, at least 65%, at least 70%, at least 75%, at least 80%, at least 85%, at least 90%, at least 95%, at least 96%, at least 97%, at least 98%, or at least 99%.

In some embodiments, the methods may detect a disease in a subject with a positive predictive value (PPV) of at least 50%, at least 55%, at least 60%, at least 65%, at least 70%, at least 75%, at least 80%, at least 85%, at least 90%, at least 95%, at least 96%, at least 97%, at least 98%, or at least 99%.

In an aspect, the methods and systems described herein may comprise monitoring the presence or susceptibility of a disease in a subject. The monitoring may comprise assess the presence or susceptibility of the disease at a plurality of time points, for example, one or more, two or more, three or more, four or more, five or more, six or more, seven or more, eight or more, nine or more, or ten or more time points. The assessing may be based at least on the presence or susceptibility of the disease determined at each of the plurality of time points.

In an aspect, the methods and systems described herein may comprise providing the subject with a therapeutic intervention or administering a treatment to the subject based at least in part on the analysis described herein. The therapeutic intervention may comprise a chemotherapy, a radiotherapy, an immunotherapy, a surgery, or a combination thereof.

In an aspect, the methods and systems herein for inferring gene expression may comprise monitoring a minimal residual disease (MRD) in a subject. The subject may be previously treated for a disease. The minimal residual disease may comprise response to treatment, tumor load, residual tumor post-surgery, relapse, secondary screen, primary screen, or cancer progression. The method may further comprise administering a treatment to the subject based on a detected change in the minimal residual disease in the subject. In some embodiments, the treatment may comprise chemotherapy, radiotherapy, immunotherapy, or surgery.

### II. Machine Learning Systems and Models

In another aspect, the present disclosure provides systems and methods comprising a classifier generated based on feature information derived from sequence analysis from biological samples of cfDNA. The classifier forms part of a predictive engine for distinguishing groups in a population based on sequence features identified in biological samples such as cfDNA.

In some embodiments, a classifier is created by normalizing the sequence information by formatting similar portions of the sequence information into a unified format and a unified scale; storing the normalized sequence information in a columnar database; training a prediction engine by applying one or more one machine learning operations to the stored normalized sequence information, the prediction engine mapping, for a particular population, a combination of one or more features; applying the prediction engine to the accessed field information to identify an individual associated with a group; and classifying the individual into a group.

The trained classifier may be configured to accept a plurality of input variables and to produce one or more output values based on the plurality of input variables. The plurality of input variables may comprise one or more datasets. For example, an input variable may comprise a number of nucleic acid sequences corresponding to or aligning to a set of genomic loci. The plurality of input variables may also include clinical health data of a subject.

A trained algorithm provided herein may comprise a classifier, such that each of the one or more output values comprises one of a fixed number of possible values (e.g., in some embodiments, a linear classifier such as, but not limited to, a logistic regression classifier, while in other embodiments, a non-linear, deep learning classifier such as, but not limited to, convolutional neural nets, etc.) indicating a classification of a sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier. The output values may comprise descriptive labels, numerical values, or a combination thereof. Some of the output values may comprise descriptive labels. Such descriptive labels may provide an identification or indication of an assessment of gene expression, and may comprise, for example, positive, negative, high-risk, intermediate-risk, low-risk, or indeterminate of gene expression. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of gene expression or gene non-expression. For example, a binary classification of samples may assign an output value of “negative” or 0 if the sample indicates that the subject has less than a 50% probability of gene expression or non-gene expression. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a gene expression or non-gene expression of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having gene expression or non-gene expression of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of gene expression or non-gene expression of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a gene expression or non-gene expression of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive,” “negative,” 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained classifier may be trained with a plurality of independent training samples. Independent training samples may be associated with gene expression or non-gene expression.

The trained classifier may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples.

The trained classifier may be trained with a first number of independent training samples associated with gene expression or non-gene expression.

The trained classifier may be configured to identify gene expression or non-gene expression at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying gene expression or non-gene expression by the trained algorithm may be calculated as the percentage of independent test samples that are correctly identified or classified as having gene expression or non-gene expression.

The trained classifier may be configured to identify gene expression or non-gene expression with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying gene expression or non-gene expression using the trained classifier may be calculated as the percentage of samples identified or classified as having gene expression or non-gene expression.

The trained classifier may be configured to identify gene expression or non-gene expression with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

In some embodiments, the model, classifier, or predictive test has a sensitivity of at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

The trained classifier may be configured to identify gene expression or non-gene expression with an Area Under the Receiver Operator Characteristic (AUROC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUROC may be calculated as an integral of the Receiver Operator Characteristic (ROC) curve (e.g., the area under the ROC curve, or AUC) associated with the trained classifier in classifying samples as having or not having gene expression or non-gene expression.

The trained classifier may be adjusted or tuned to improve one or more of the performance, accuracy, PPV, NPV, clinical sensitivity, clinical specificity, or AUC of identifying gene expression or non-gene expression. The trained classifier may be adjusted or tuned by adjusting parameters of the trained classifier (e.g., a set of cutoff values used to classify a sample as disclosed elsewhere herein, or weights of a neural network). The trained classifier may be adjusted or tuned continuously during the training process or after the training process has completed.

After the trained classifier is initially trained, a subset of the inputs may be identified as most influential or most important to be included for making high-quality classifications. For example, a subset of the plurality of input variables may be identified as most influential or most important to be included for making high-quality classifications or identifications of assessments of gene expression or non-gene expression. The plurality of input variables or a subset thereof may be ranked based on classification metrics indicative of each input variable's influence or importance toward making high-quality classifications or identifications of assessments of the gene expression or non-gene expression. Such metrics may be used to reduce, in some cases significantly, the number of input variables (e.g., predictor variables) that may be used to train the trained classifier to a desired performance level (e.g., based on a desired minimum accuracy, PPV, NPV, clinical sensitivity, clinical specificity, AUC, or a combination thereof). For example, if training the trained classifier with a plurality comprising several dozen or hundreds of input variables in the trained classifier results in an accuracy of classification of more than 99%, then training the trained classifier instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

In some embodiments, the subject matter disclosed herein can include a digital processing device or use of the same. In some embodiments, the digital processing device can include one or more hardware central processing units (CPU), graphics processing units (GPU), or tensor processing units (TPU) that carry out the device's functions. In some embodiments, the digital processing device can include an operating system configured to perform executable instructions. In some embodiments, the digital processing device may be connected a computer network. In some embodiments, the digital processing device may be connected to the Internet. In some embodiments, the digital processing device may be connected to a cloud computing infrastructure. In some embodiments, the digital processing device may be connected to an intranet. In some embodiments, the digital processing device may be connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some embodiments, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD®, Linux, Apple® Mac OS X Server®, Oracle® Solaris® Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some embodiments, the operating system can be provided by cloud computing, and cloud computing resources can be provided by one or more service providers.

In some embodiments, the device can include a storage and/or memory device. The storage and/or memory device can be one or more physical apparatuses used to store data or programs on a temporary or permanent basis. In some embodiments, the device can be volatile memory and require power to maintain stored information. In some embodiments, the device can be non-volatile memory and retain stored information when the digital processing device is not powered. In some embodiments, the non-volatile memory can include flash memory. In some embodiments, the non-volatile memory can include dynamic random-access memory (DRAM). In some embodiments, the non-volatile memory can include ferroelectric random-access memory (FRAM). In some embodiments, the non-volatile memory can include phase-change random access memory (PRAM). In some embodiments, the device can be a storage device including, for example, CD-ROMs, DVDs, flash memory devices, magnetic disk drives, magnetic tapes drives, optical disk drives, and cloud computing-based storage. In some embodiments, the storage and/or memory device can be a combination of devices such as those disclosed herein. In some embodiments, the digital processing device can include a display to send visual information to a user. In some embodiments, the display can be a cathode ray tube (CRT). In some embodiments, the display can be a liquid crystal display (LCD). In some embodiments, the display can be a thin film transistor liquid crystal display (TFT-LCD). In some embodiments, the display can be an organic light emitting diode (OLED) display. In some embodiments, an OLED display can be a passive-matrix OLED (PMOLED) or active-matrix OLED (AMOLED) display. In some embodiments, the display can be a plasma display. In some embodiments, the display can be a video projector. In some embodiments, the display can be a combination of devices such as those disclosed herein.

In some embodiments, the digital processing device can include an input device to receive information from a user. In some examples, the input device can be a keyboard. In some embodiments, the input device can be a pointing device including, for example, a mouse, trackball, trackpad, joystick, game controller, or stylus. In some embodiments, the input device can be a touch screen or a multi-touch screen. In some embodiments, the input device can be a microphone to capture voice or other sound input. In some embodiments, the input device can be a video camera to capture motion or visual input. In some embodiments, the input device can be a combination of devices such as those disclosed herein.

In some embodiments, the subject matter disclosed herein can include one or more non-transitory computer-readable storage media encoded with a program including instructions executable by the operating system. The operating system may be part of a networked digital processing device. In some examples, a computer-readable storage medium can be a tangible component of a digital processing device. In some embodiments, a computer-readable storage medium may be removable from a digital processing device. In some embodiments, a computer-readable storage medium can include, for example, CD-ROMs, DVDs, flash memory devices, solid state memory, magnetic disk drives, magnetic tape drives, optical disk drives, cloud computing systems and services, and the like. In some embodiments, the program and instructions can be permanently, substantially permanently, semi-permanently, or non-transitorily encoded on the media.

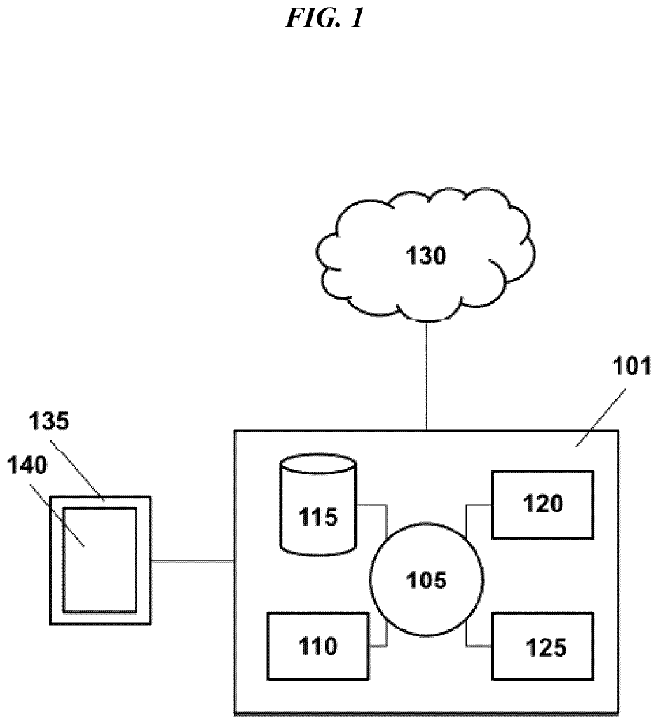

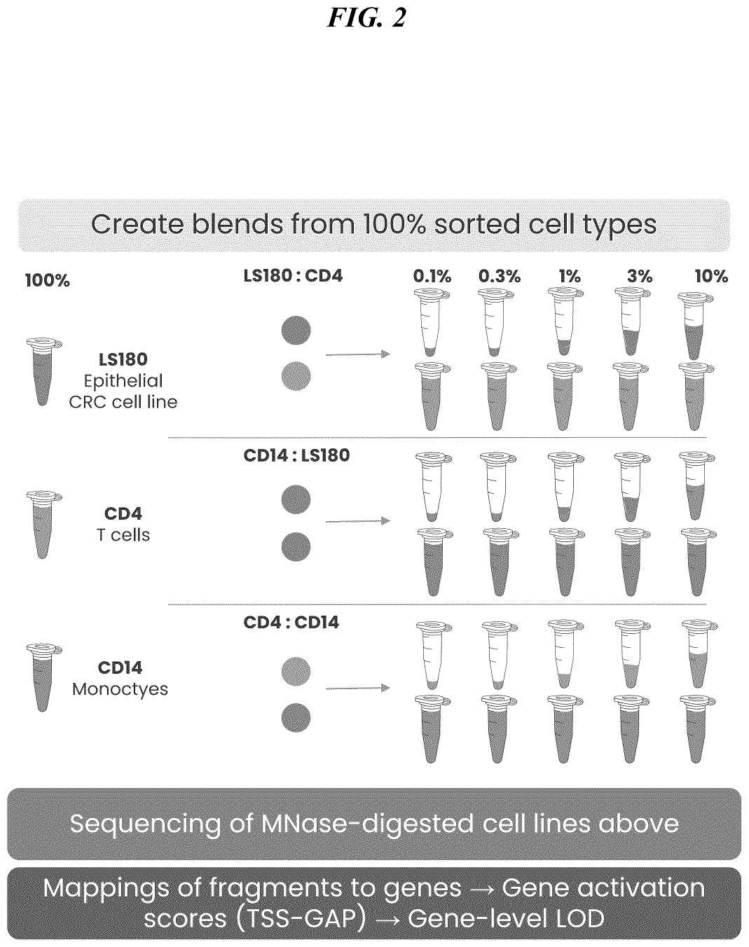

The present disclosure provides computer systems that are programmed to implement methods disclosed herein. FIG. 1 shows a computer system 101 that is programmed or otherwise configured to perform methods of the present disclosure, such as storing, processing, identifying, or interpreting subject (e.g., patient) data, biological data, biological sequences, reference sequences, or features. The computer system 101 can process various aspects of subject (e.g., patient) data, biological data, biological sequences, or reference sequences of the present disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some embodiments is a telecommunication and/or data network. The network 130 can include one or more computer servers, which can enable distributed computing, such as cloud computing. The network 130, in some embodiments with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some examples, the circuit is an application specific integrated circuit (ASIC).

The storage unit 115 can store files, such as drivers, libraries and saved programs. The storage unit 115 can store user data, e.g., user preferences and user programs. The computer system 101 in some embodiments can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as disclosed herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some examples, the code can be retrieved from the storage unit 115 and stored on the memory 110 for ready access by the processor 105. In some embodiments, the electronic storage unit 115 can be precluded, and machine-executable instructions are stored on memory 110.

The code can be pre-compiled and configured for use with a machine having a processer adapted to execute the code or can be interpreted or compiled during runtime. The code can be supplied in a programming language that can be selected to enable the code to execute in a pre-compiled, interpreted, or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a nucleic acid sequence, an enriched nucleic acid sample, an expression profile, and an analysis of an expression profile. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, probe a plurality of regulatory elements, sequence a nucleic acid sample, enrich a nucleic acid sample, determine an expression profile of a nucleic acid sample, analyze an expression profile of a nucleic acid sample, and archive or disseminate results of analysis of an expression profile.

In some examples, the subject matter disclosed herein can include at least one computer program or use of the same. A computer program can be a sequence of instructions, executable in the digital processing device's CPU, GPU, or TPU, written to perform a specified task. Computer-readable instructions can be implemented as program modules, such as functions, objects, Application Programming Interfaces (APIs), data structures, and the like, that perform particular tasks or implement particular abstract data types. For example, a computer program can be written in various versions of various languages.

The functionality of the computer-readable instructions can be combined or distributed as desired in various environments. In some examples, a computer program can include one sequence of instructions. In some examples, a computer program can include a plurality of sequences of instructions. In some examples, a computer program can be provided from one location. In some examples, a computer program can be provided from a plurality of locations. In some examples, a computer program can include one or more software modules. In some examples, a computer program can include, in part or in whole, one or more web applications, one or more mobile applications, one or more standalone applications, one or more web browser plug-ins, extensions, add-ins, or add-ons, or combinations thereof.

In some examples, the computer processing can be a method of statistics, mathematics, biology, or any combination thereof. In some examples, the computer processing method includes a dimension reduction method including, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, matrix factorization, network clustering, and neural network.

In some examples, the computer processing method is a supervised machine learning method including, for example, a regression, support vector machine, tree-based method, and network.

In some examples, the computer processing method is an unsupervised machine learning method including, for example, clustering, network, principal component analysis, and matrix factorization.

### EXAMPLES

### Example 1: Inference of Gene Expression Using Fragmentation Patterns from Targeted High-Depth Sequencing of Cell-Free DNA

Using methods and systems of the present disclosure, a targeted sequencing approach is developed that enables individual gene expression inference from cfDNA fragmentation patterns based on transcription start-site gene activation probability (TSS-GAP).

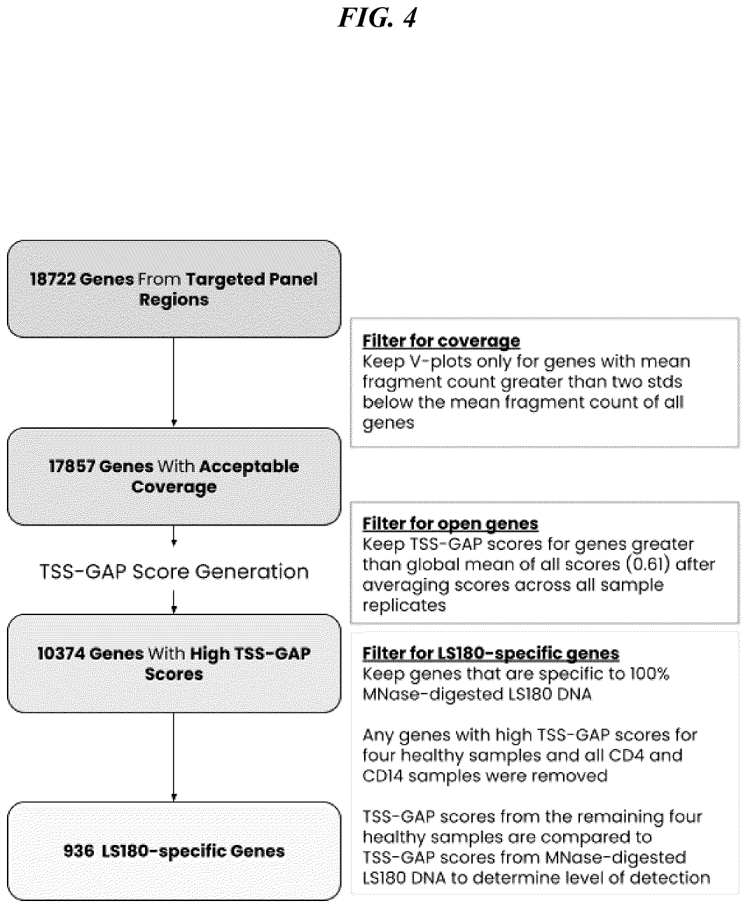

To assess the sensitivity of this approach for cfDNA-inferred gene expression profiling, TSS-GAP was applied to blends of micrococcal nuclease (MNase)-digested DNA from a cancer cell line (LS180) and sorted peripheral blood mononuclear cells (PBMC), with LS180 DNA at varying concentrations (0.1-10%). This enabled the determination of the limit of detection (LoD), relative to healthy donor background levels, on the basis of the genes' activation probabilities (TSS-GAP scores) as shown in FIG. 4.

As provided in FIG. 4, 936 genes were observed with high inferred expression in the LS180 cancer cell line and low inferred expression in non-cancer samples (e.g., CD4, CD14, and plasma cfDNA from 4 healthy donors) and these genes were used for healthy donor background and LoD assessment. Using these gene-level background values as cutoffs, blends were assessed to determine the lowest blend level at which expression of the gene was able to be differentiated from background. Out of the 936 genes, 326 (34.8%) were detectable at a blend level of 0.1%, 59 (6.3%) at 0.3%, 31 (3.3%) at 1%, 19 (2.0%) at 3%, and 39 (4.2%) at 10%. In total, 416 genes were detected by TSS-GAP at levels 1% of or lower above background, which highlighted that this approach for detecting expression of certain genes even from a small fraction of cells contributing cfDNA to liquid biopsy samples.

### Example 2: Validation of Fragmentation-Based Gene Expression Inference Using Sorted Cell Lines

### 1. BACKGROUND

Cell-free DNA (cfDNA) contains epigenetic signatures of the cells from which it was produced. As a result, cfDNA enables gene activation inference and tissue-of-origin classification with potential applications for non-invasive cancer detection. However, because of low depth of coverage of sites of interest, current whole genome sequencing (WGS) methods and techniques have not been able to infer expression of individual genes or limited gene sets. Additionally, low tumor cell contribution in cfDNA increases the difficulty of detecting tumor signals above healthy background.

Using methods and systems of the present disclosure, the computational method described herein was developed to measure transcription start site gene activation probabilities (referring to herein as “TSS-GAP”) using a targeted deep sequencing approach. The sensitivity of this computational method was measured by analyzing results of micrococcal nuclease (MNase)-digested nuclei and cfDNA. Micrococcal nuclease is an endo-exonuclease that digests nucleic acids.

### 2. OBJECTIVES

One objective of the methods disclosed herein is to determine sets of expressed genes from TSS-GAP scores specific to a cancer cell line while removing all traces of healthy signal.

An additional objective disclosed herein is to determine gene-level limit of detection (LoD) of TSS-GAP by comparing TSS-GAP scores associated with cancer signal to those associated with healthy donor-derived signal among the prior sets of genes.

### 3. METHODS

Micrococcal nuclease (MNase)-digested DNA has been shown to produce nucleosome-bound fragments comparable to cfDNA.

cfDNA from healthy donor plasma (n=8) served as the healthy background signal while blends of MNase-digested DNA from a CRC epithelial cell line (LS180) and sorted immune cell populations (CD4 and CD14) at varying concentrations (n=18) served as contributing tumor and immune cell signal that can be found in cfDNA

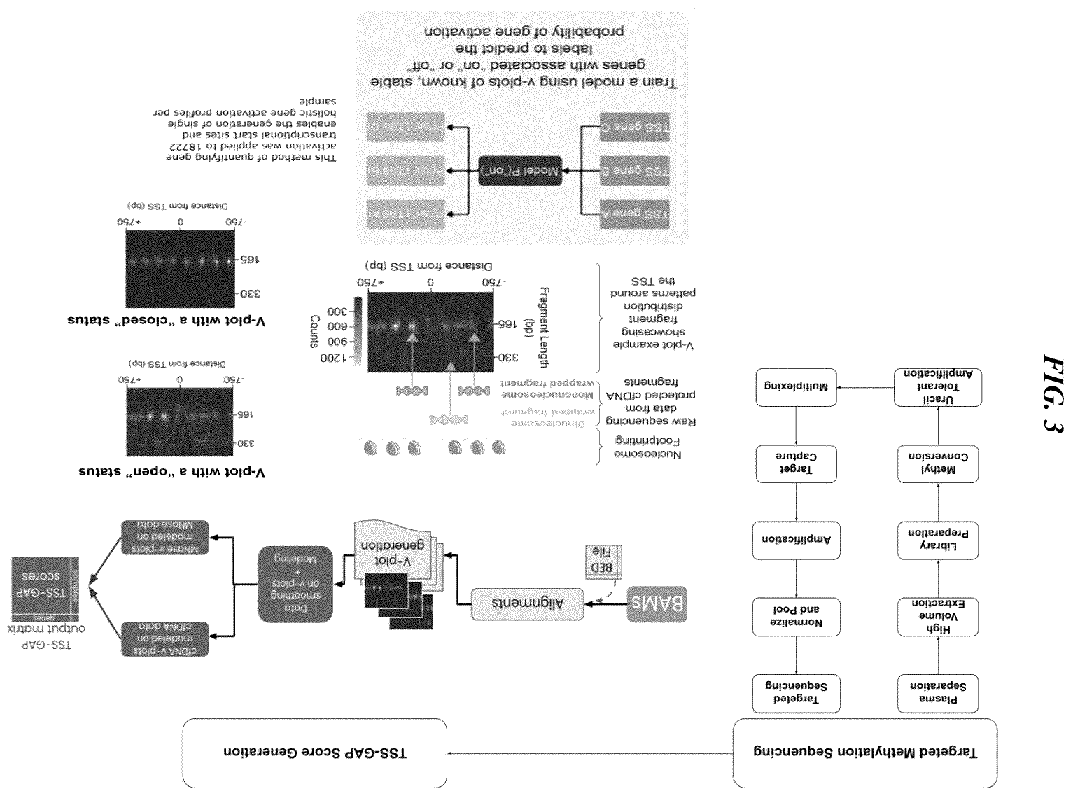

As shown in FIG. 3, gene activation was predicted from plasma cell-free DNA (cfDNA) using both fragment length and fragment position.

TSS-GAP scores ranged from 0-1, where a score of 0 indicates the lowest possible activation score and a score of 1 indicates the highest possible activation score.

BAM files were downloaded and prepared for alignment. BED files were further added for alignment. V-plots, which are two-dimensional plots of paired-end sequencing fragments from chromatin accessibility assays (e.g., MNase-seq), are generated. cfDNA is known to correspond to regions of the genome that are protected by proteins. Paired-end sequencing of cfDNA provides fragment lengths and recovers protected fragments of DNA. For an average V-plot of an expressed “on” gene, DNA-protein binding location and binding-site size can be inferred from fragment length and location (genomic position) of sequenced cfDNA fragments. To generate the V-plots, the number of fragments is counted from a single-nucleotide base pair region (from a BED file) outward-750 bp to +750 bp in 33 bp bins. This region can encompass all fragments from 16 bp up to a maximum size of 400 bp (e.g., can be counted in 16 bp bins). This fragment counting method is done across all regions of the BED file. What is outputted are 4D arrays (the V-plots) with the corresponding dimensions: (sample, region in bed file, fragment size, fragment position).

Each pixel in the V-plot is colored by how many fragments with a particular length (y-axis) have a midpoint at this position (x-axis) and darker colors on the V-plot indicate a larger number of fragments. The V-plots were smoothed and modeled. A machine learning model was trained to determine whether a gene is “on” or “off” in cfDNA producing cells. As described herein, the candidate “off” genes were selected from Table 1 and the “on” genes were selected from Table 2. The machine learning model was trained on the average expression of stable genes from external datasets. The V-plots were generated using different data types, such as cfDNA data for cfDNA v-plots and MNase-treated DNA data for MNase V-plots. Modeling was then performed on cfDNA V-plots for cfDNA data and modeling was performed on MNase V-plots for MNase data. TSS-GAP scores were generated by a TSS-GAP output matrix, where the x-axis represented genes and the y-axis represented samples.

As shown in FIG. 4, LS180-specific open genes were extracted through the blacklisting of healthy-specific genes for determining the limit of detection (LoD). To extract LS180-specific open genes, V-plots for genes with reasonable coverage (e.g., based on overall fragment count) were analyzed and generated a TSS-GAP score. The genes with TSS-GAP scores greater than the global mean of all scores (e.g., 0.61 (scores for each sample were averaged across replicates)) were retained. Genes that were not specific to 100% MNase-digested LS180 DNA were filtered out (e.g., and the remaining list of genes should be colorectal cancer-specific genes and should not include healthy- or immune-contributed signal). Lastly, limit of detection (LoD) was obtained by comparing the associated TSS-GAP scores between MNase-digested LS180 DNA and healthy donor-derived cfDNA.

### 4. RESULTS

FIG. 5 shows generated V-plots by the methods described herein for the genes FOXA1 and MUC6 at various percentages of LS180 (e.g., 100%, 90%, 1%) and at a healthy state. The openness of the V-plots reflects TSS-GAP score profiles for healthy donor-derived cfDNA and LS180 dilutions (e.g., 100%, 90%, 1%).

Still referring to FIG. 5, At 1% LS180, expression signal in FOXA1 is detected while expression signal in MUC6 is not from their respective TSS-GAP scores (FOXA1 score: 0.73, MUC6 score: 0.02). This can be traced back to a difference in fragment length distributions at the TSS from the genes' associated V-plots. The V-plot for FOXA1 shows a finer open “V” pattern with fragments spanning 120 base pairs to 210 base pairs at the TSS that help the machine learning model deem it to be open. The V-plot for MUC6 lacks the proper open “V” pattern with minimal fragments spanning 144 base pairs to 200 base pairs as the TSS makes it hard for the TSS-GAP model to deem it open or closed.

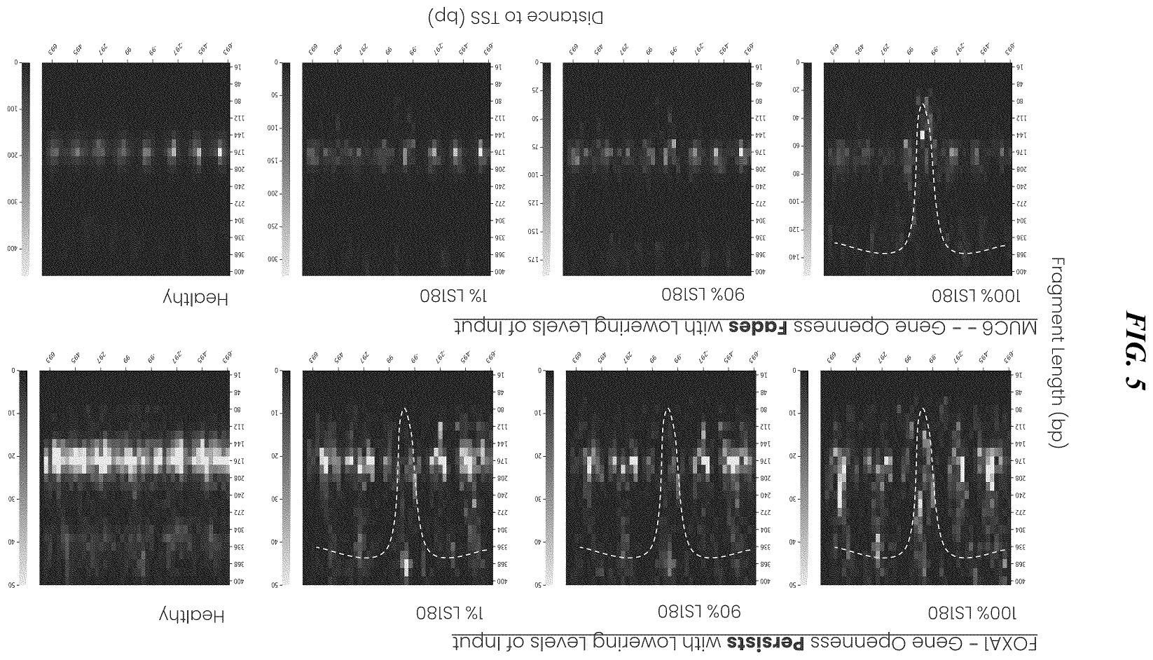

Referring to FIG. 6, TSS-GAP scores in LS180 dilutions within LS180 specific genes were observed to be higher than those of non-blacklisted healthy samples.

Genes observed with the 15 best limit of detection (LoD) (e.g., the greatest separation between mean LS180 TSS-GAP score and max healthy score) showed TSS-GAP can distinguish specific signals from one cell type from another at significantly low dilutions. The 15 genes, as shown in FIG. 5, include SOWAHB, TMEM63C, SOX2, TMEM184A, NBL1, B4GALNT2, TFAP2B, RND2, TP63, ATG9B, IGSF9, TMEM82, C10orf99, LOXL1, and GRB7.

Several genes are expressed in colorectal tissue and epithelial cells e.g., TP63 and SOX2. Round dots represent “open” and highly-expressed LS180 scores; the darker the color, the more concentrated the sample. Boxplots represent non-blacklisted healthy scores.

TABLE 6
LS180-specific genes detectable above
healthy at each concentration.

| | Concentration of LS180 | | | | | | Total |
|---|---|---|---|---|---|---|---|
| | 0.10% | 0.30% | 1.0% | 3.0% | 10.0% | None | genes |
| # genes | 326 | 385 | 416 | 435 | 474 | 462 | 936 |
| detectable | | | | | | | |
| at | | | | | | | |
| each | | | | | | | |
| concentration | | | | | | | |
| of LS180: | | | | | | | |

Limit of Detection (LoD) may be referred to as the number of genes that are specific to each dilution level. Numbers of genes in the table are cumulative across concentrations of LS180. Detectability is determined if the minimum TSS-GAP score for a gene at a concentration of LS180 is greater than the mean TSS-GAP score for the same gene among cfDNA from four healthy donors.

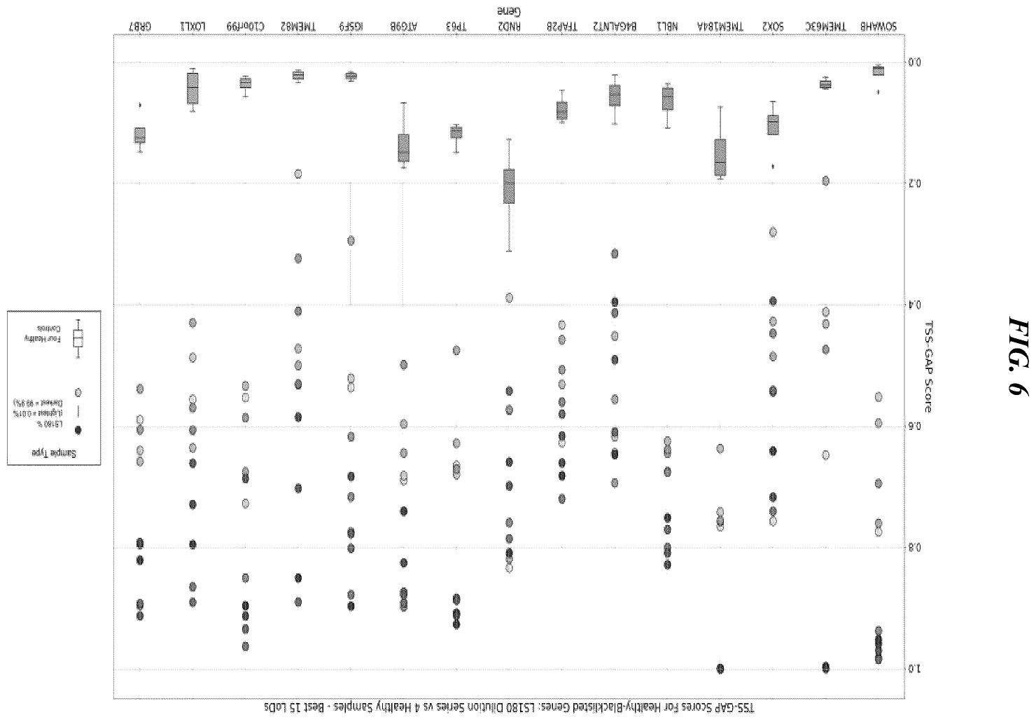

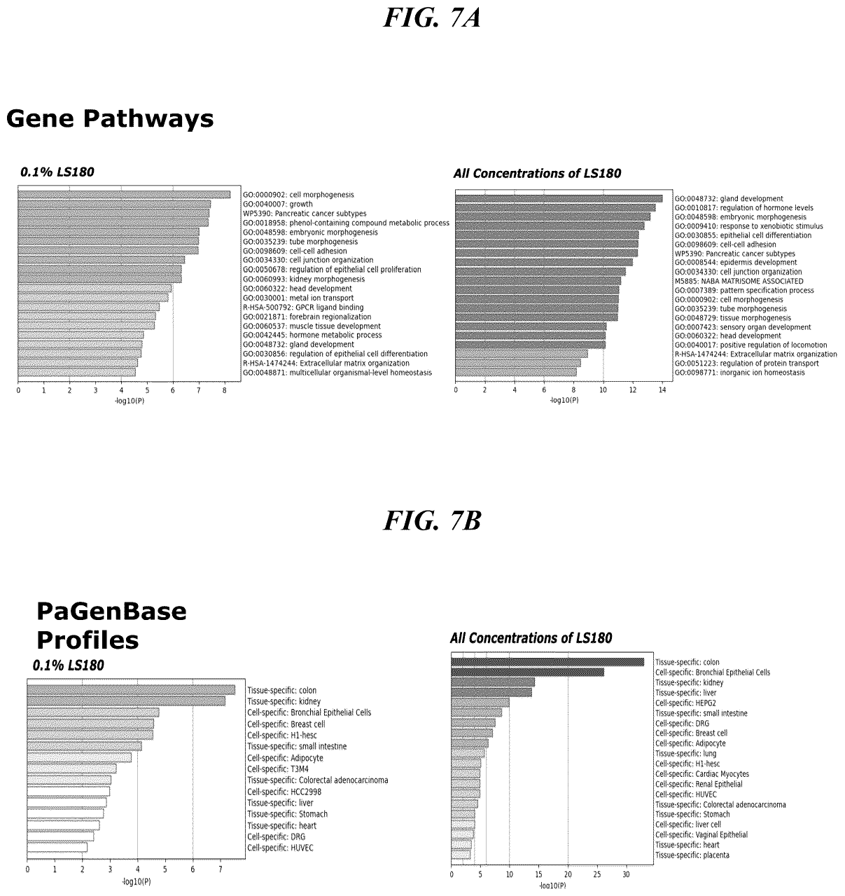

FIGS. 7A-7B show graphs illustrating the most enriched gene pathways and PaGenBase gene profiles in 0.1% LS180 and all concentrations of LS180 (see Table 6).

Referring to FIGS. 7A-7B, bar graphs depicting the top enriched gene pathways and PaGenBase gene profiles in 0.1% LS180 and all concentrations of LS180 ranked most enriched to least enriched by-log (p-adjusted). Gene pathways were gathered from the following ontology sources: KEGG Pathway, GO Biological Processes, Reactome Gene Sets, Canonical Pathways, CORUM, WikiPathways, and PANTHER Pathway. PaGenBase gene profiles were gathered from the PaGenBase database.

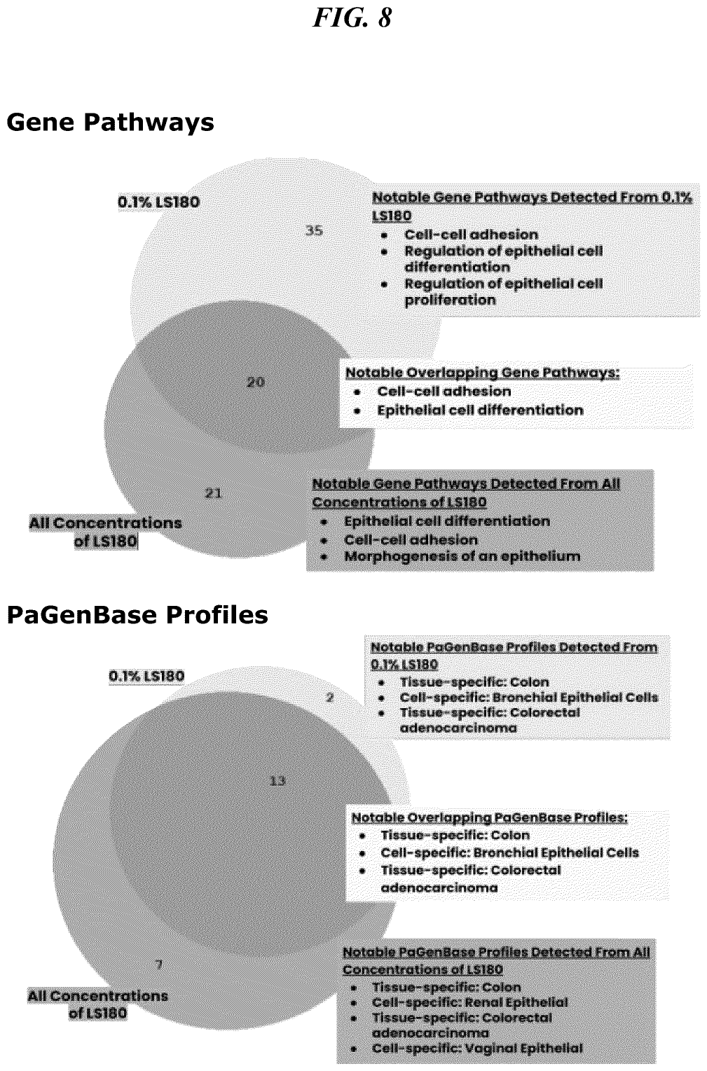

FIG. 8, a pair of Venn diagrams showing notable overlapping gene pathways and profiles depicted in FIGS. 7A-7B between 0.1% LS180 and all concentrations of LS180.

TSS-GAP was able to detect epithelia colorectal cancer (CRC)-specific pathways and gene expression profiles at higher concentrations of LS180 (epithelial cell differentiation, colon tissue/epithelial cell signatures at all concentrations). TSS-GAP was also able to detect such relevant information at significantly lower concentrations of LS180 (cell-cell adhesion, regulation of epithelial cell proliferation, colorectal adenocarcinoma tissue signatures and epithelial cell signatures at 0.1%).

### 5. CONCLUSIONS

The method presented herein describes the methods for TSS-GAP as well as how to assess the sensitivity of TSS-GAP at the gene level.

Thus, an LoD for TSS-GAP can be obtained on the individual gene level. For example, LS180 signal can be detected above healthy cfDNA signal at very low concentrations of LS180-down to 0.1%

In certain embodiments, colorectal cancer epithelial lineage genes can be detected at very low levels (0.1%) by TSS-GAP

### Example 3: Methods and Systems for Generating Tss-Gap Scores

cfDNA-based pipelines

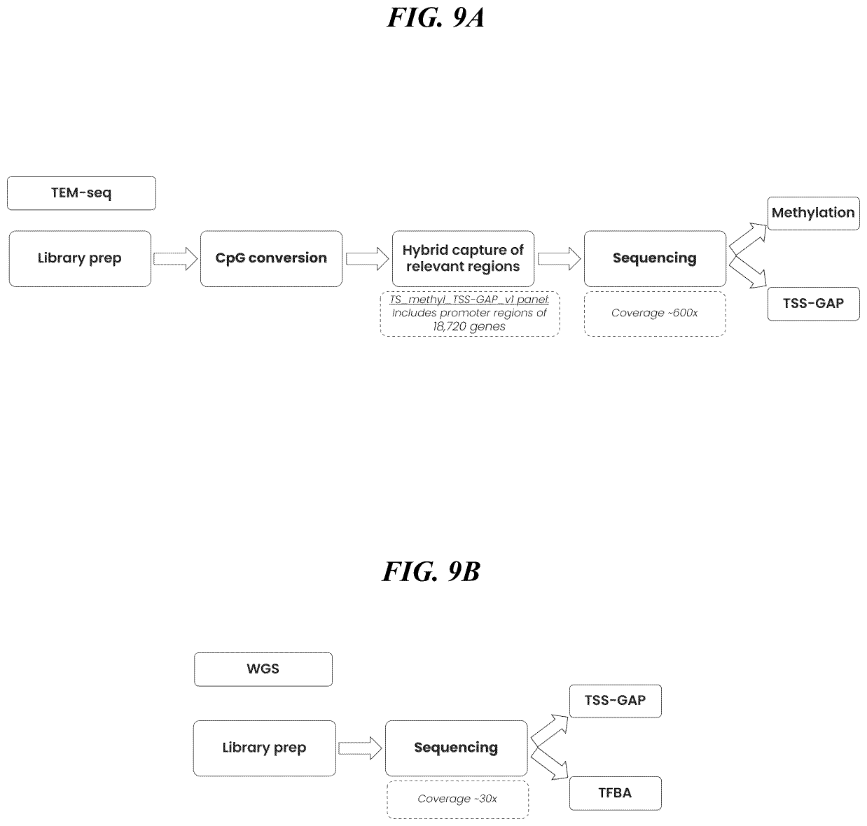

FIG. 9A illustrates an example of a workflow used in the methods and systems described herein. As shown in FIG. 9A, DNA (e.g., cell-free DNA, nuclease-treated DNA, fragmented DNA, etc.) is used to generate a sequencing library (library prep). An enzymatic conversion operation is performed (CpG conversion) as described herein (e.g., EM-seq). A hybrid capture panels that include regions flanking transcription start site (TSS) sequences of a panel of genes are provided. For example, the hybrid capture panels may target flanking sequences that are 750 base pairs upstream and 750 base pairs downstream of a TSS sequence. Target genes may include the genes provided in any of Tables 1-5 as described herein. In one embodiment, target genes may include the genes provided in Table 1. In another embodiment, target genes may include the genes provided in Table 2. In yet another embodiment, target genes may include the genes provided in Table 3. In still another embodiment, target genes may include the genes provided in Table 4. In another embodiment, target genes may include the genes provided in Table 5. Sequencing at a coverage of about 600×is performed using an Illumina sequencer to generate reads for methylation, transcription start site gene activation probability (TSS-GAP), or a combination thereof.

FIG. 9B illustrates an example of a workflow used in the methods and systems described herein. As shown in FIG. 9B, DNA (e.g., cell-free DNA, aminase-treated DNA, fragmented DNA, etc.) is used to generate a sequencing library (library prep). Sequencing at a coverage of about 30×is performed to generate reads for TSS-GAP, transcription factor binding accessibility (TFBA), or a combination thereof. Computational analysis of the methylation sequencing, whole genome sequencing, TSS-GAP, and/or TFBA shown in FIGS. 9A-9B can be conducted to generate various computational readouts used in the methods and systems described herein.

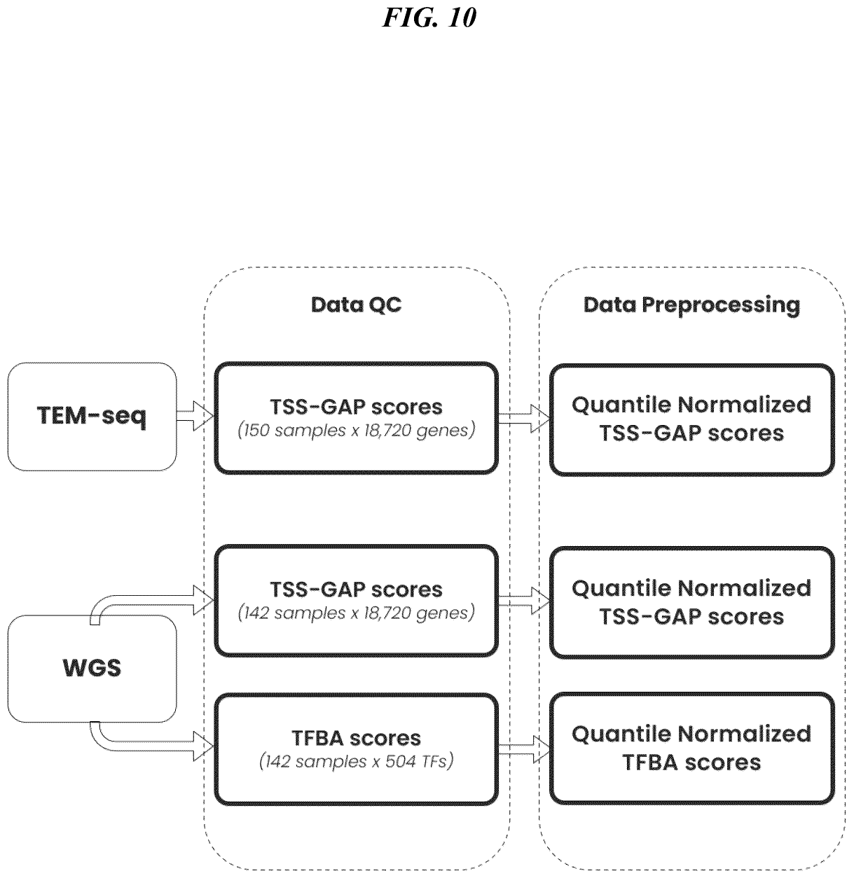

From the TEM-seq and WGS workflows illustrated in FIGS. 9A-9B, TSS-GAP scores are generated (data QC and data preprocessing), as shown in FIG. 10.

Dna Fragmentation Profiles Associated with cfDna and MNase-Digestion

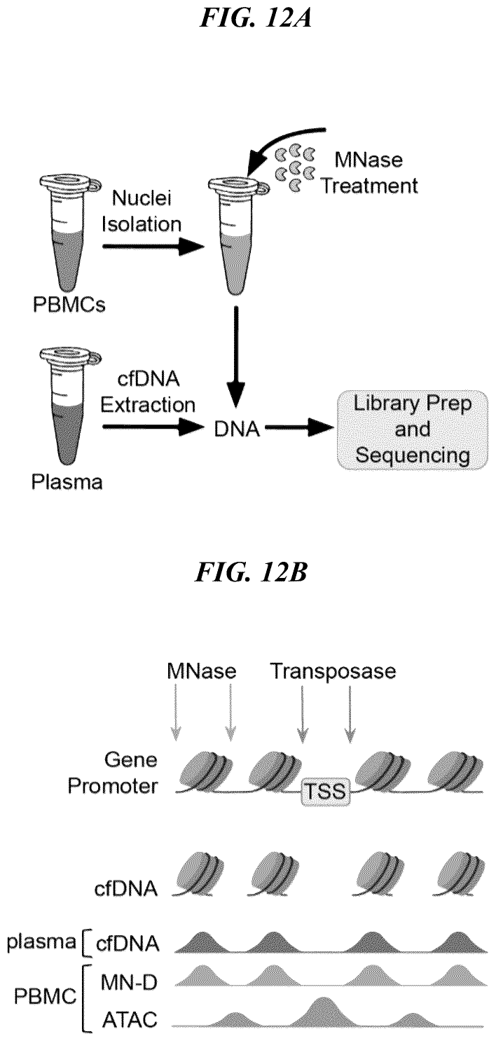

FIG. 12A shows an alternative workflow for processing samples to generate DNA from Peripheral Blood Mononuclear Cells (PBMCs) for library prep and sequencing. In a first workflow, Peripheral Blood Mononuclear Cells are collected, nuclei are isolated from the PBMCs, and the resulting isolate is treated with MNase. In some cases, circulating blood cells, tumor cfDNA, cell lines, monocytes, t-cells, LS180 cell line, plasma, or a combination thereof, may be used. Still referring to FIG. 12A, in a second workflow, plasma is collected and cfDNA is extracted from the plasma. In some cases, previously extracted cfDNA is obtained. Fragmentation patterns are generated from the DNA from the first workflow (“PBMC/MNase”) and the second workflow (“cfDNA”).

FIG. 12B shows a comparison between the fragmentation patterns from the PBMC/MNase workflow and the cfDNA workflow. As shown in FIG. 12B, a similar fragmentation pattern is observed from the PBMC/MNase workflow and the cfDNA workflow. As such, the PBMC/MNase workflow method is validated as comparable to the cfDNA workflow method.

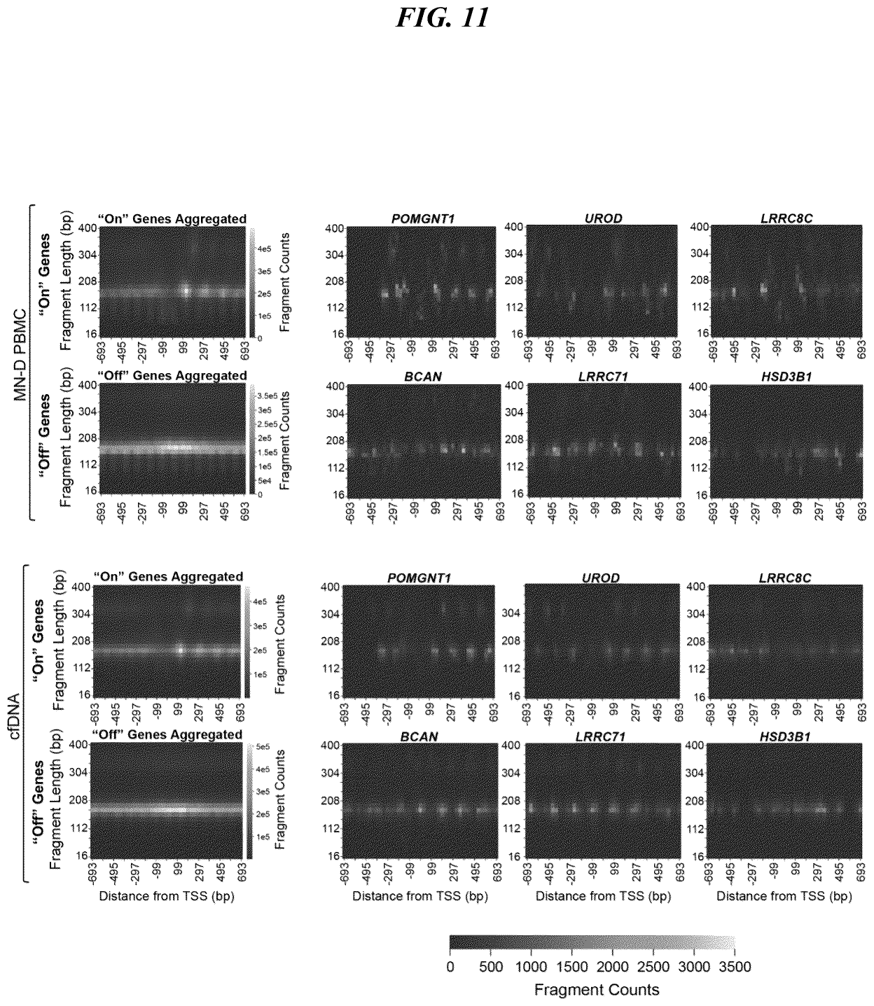

1,190 training genes are used to train a machine learning model to determine whether a gene is likely to be expressed or non-expressed using the methods described herein. A list of 595 genes was created, and these genes are known to be off (Table 1). These genes are typically off in tissue and were cross referenced with FANTOM. A list of 595 genes was created, and these genes are known to be constitutively on (Table 2). These genes are mostly housekeeping genes and were cross referenced with APPRIS. FIG. 11 illustrates v-plots for 6 representative genes (POMGNT1, UROD, LRRC8C, BCAN, LRRC71, and HSD3B1). The representative “on” genes include POMGNT1, UROD, and LRRC8C. The representative “off” genes include BCAN, LRRC71, and HSD3B1. As shown in FIG. 11, when a gene is “on” there is less saturation in the middle of the v-plot around the TSS, there are fewer fragments, and the chromatin is observed to be opened. When a gene is “off” there is more saturation in the middle of the v-plot around the TSS, which shows that there are many fragments, and the chromatin is observed to be closed. FIG. 11 (top) shows v-plots from the PBMC/MNase workflow described in FIG. 12A and FIG. 11 (bottom) shows v-plots from the cell-free DNA workflow described in FIG. 12A.

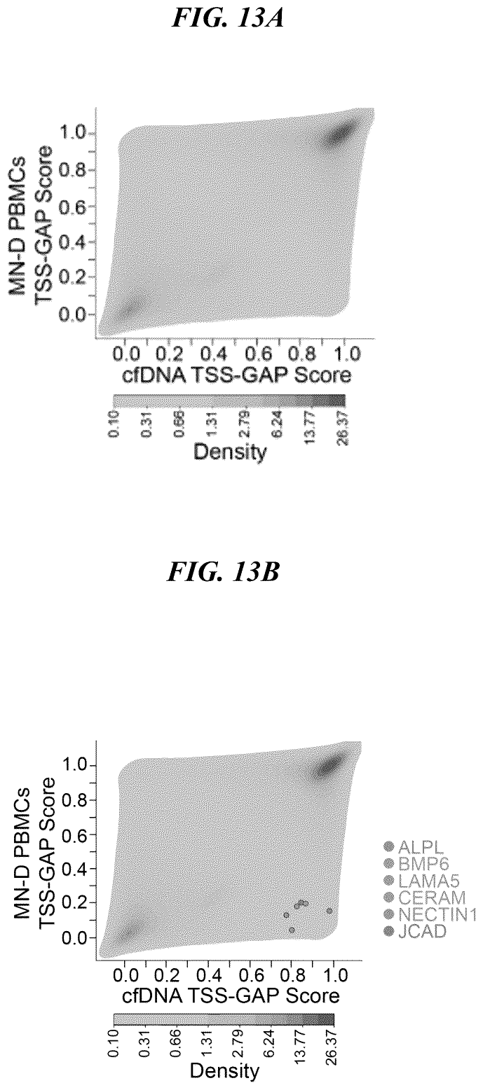

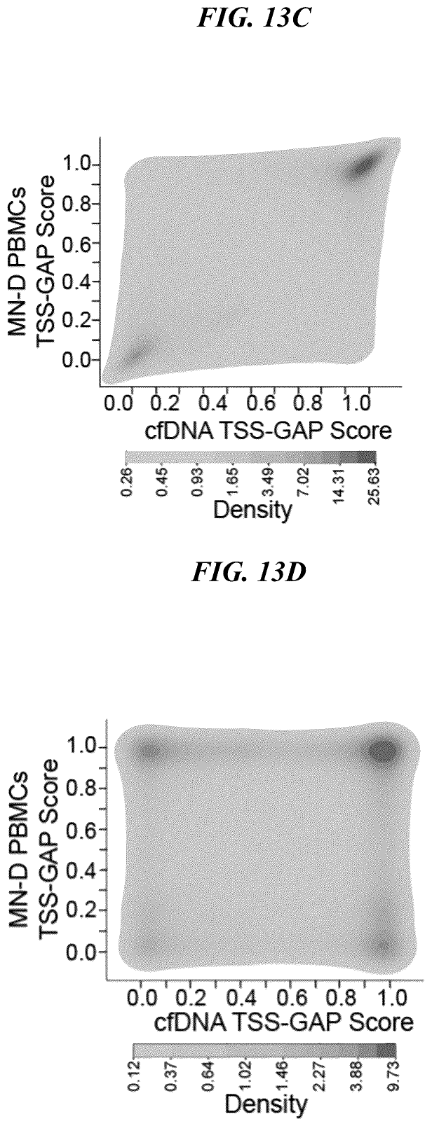

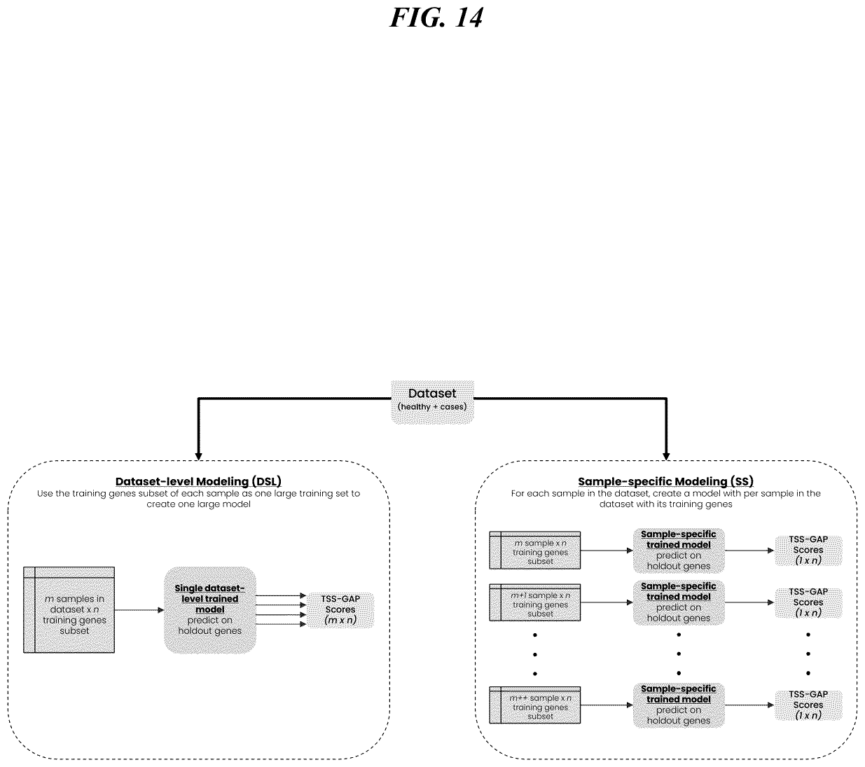

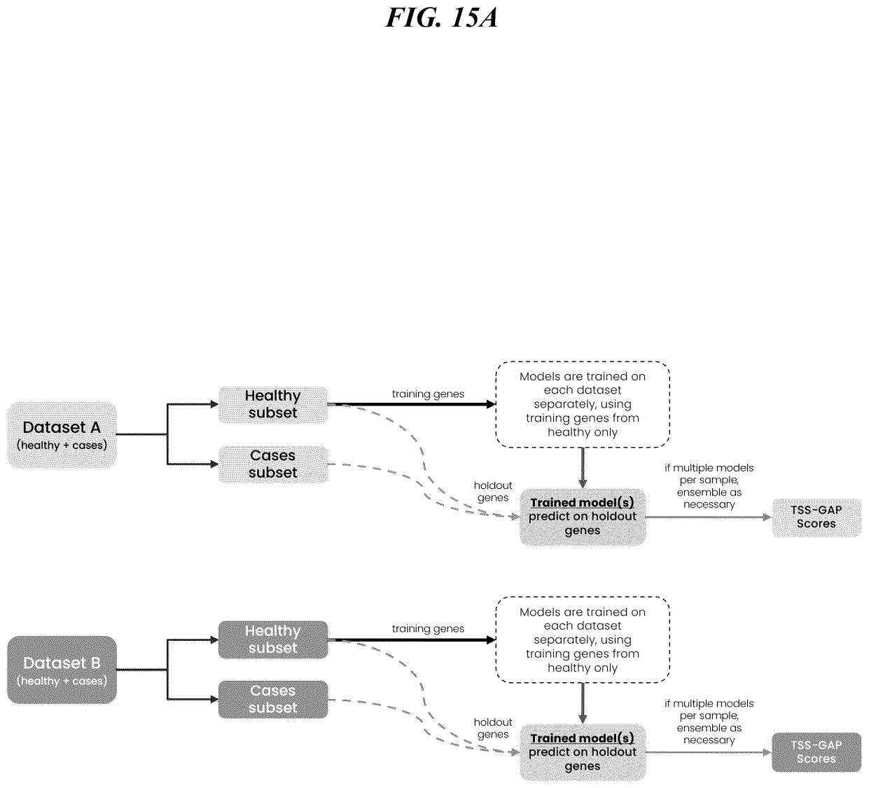

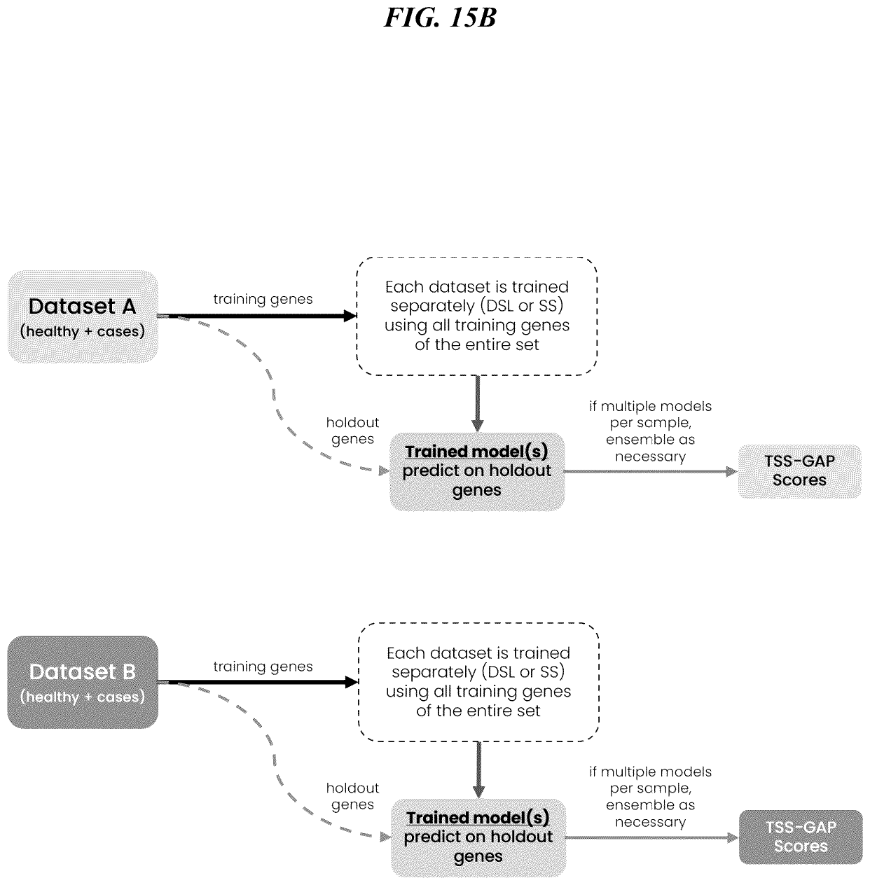

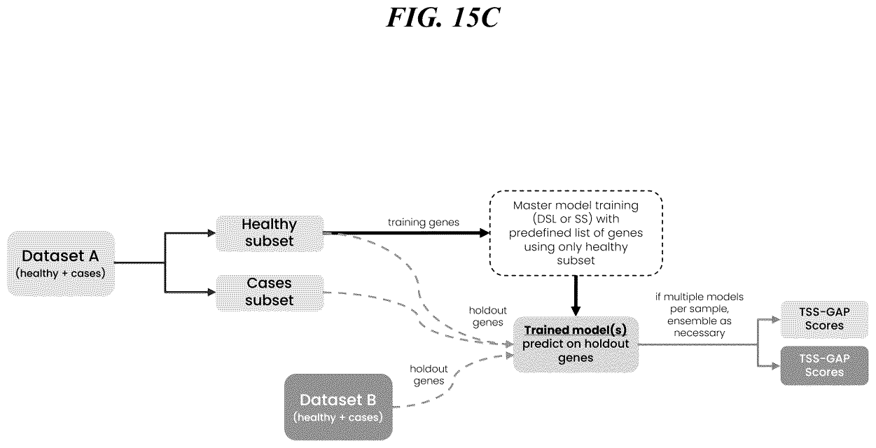

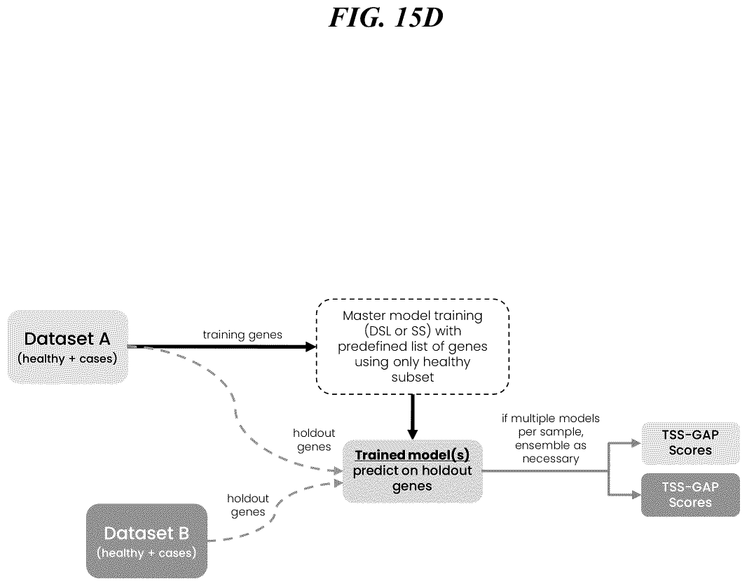

FIGS. 13A-D illustrate scatter plots comparing the TSS-GAP scores of the PBMC/MNase workflow and the cfDNA workflow illustrated in FIG. 12A. As shown in FIGS. 13A-D, both workflows/methods show comparable TSS-GAP scores.

FIGS. 13A-13B show that there was high concordance between the PBMC/MNase workflow and the cfDNA workflow. FIG. 13B validated that certain epithelial genes (e.g., ALPL, BMP6, LAMA5, CERAM, NECTIN1, and JCAD) that were expected to be high in the cfDNA workflow and low in the PBMC/MNase workflow showed results as expected.

In FIG. 13C, the samples were scrambled, and the genes were matched. FIG. 13C shows a strong correlation between the PBMC/MNase workflow and the cfDNA workflow.

In FIG. 13D, the samples were matched, and the genes were scrambled. The clustering around the four corners in FIG. 13D illustrates that the correlation was not as strong between the PBMC/MNase workflow and the cfDNA workflow.

Cfdna Extraction from Plasma

Cell-free DNA was extracted from plasma using magnetic beads (e.g., Omega Mag-Bind cfDNA kit). Briefly, the plasma samples were treated with ProteinaseK at 60° C. to remove any contaminating proteins, followed by binding of DNA to the magnetic beads. The bead-bound DNA was then purified using sequential washes and eluted in Elution Buffer. The eluted cfDNA was quantified using the 5400 Fragment Analyzer System with the HS Large Fragment Kit (Agilent).

MNase Digestion and DNA Isolation from PBMCs

Nuclei isolation, MNase treatment, and nucleosomal DNA purification was performed on PBMCs with the EZ Nucleosomal DNA Prep Kit (Zymo) according to the manufacturer instructions with minor modifications. Specifically, the PBMCs were freshly thawed, and the number of live cells counted with the Countess 3 Automated Cell Counter (Invitrogen). MNase digestion was performed on ˜500,000 live cells with 0.5 U of MNase at 25° C.° C. for 5 minutes. After purification, the size distribution and concentration of the resulting DNA was assessed using the 5400 Fragment Analyzer System with the HS Large Fragment Kit (Agilent).

Methyl Conversion of cfDNA and/or DNA Isolated from PBMCs

The extracted cfDNA or DNA isolated from PBMCs is used to generate a sequencing library (library prep). The DNA may include cell-free DNA. The DNA may include nuclease-treated DNA, for example, MNase treated DNA. The DNA may include fragmented DNA. After library prep, enzymatic conversion is performed as described herein, which may comprise CpG conversion, enzymatic methyl-seq (EM-seq), or a combination thereof on the sequencing libraries. Examples of enzymatic methyl conversion operations that may be used include enzymatic methyl-seq (EM-seq) and TET-assisted pyridine borane sequencing (TAPS).

EM-seq is a minimally destructive conversion methylation sequencing method for converting cytosines to uracils in nucleic acids. This bisulfite-free method preserves the length of nucleic acid molecules while achieving conversion rates similar to bisulfite sequencing. Further, EM-Seq can result in higher sequencing quality scores for cytosine and guanine base pairs, and can provide a more even coverage of various genomic features, such as CpG islands. EM-Seq may comprise two sets of enzymatic reactions. In a first reaction, a ten eleven translocation (TET) enzyme (e.g., TET1, TET2, TET3, Naegleria TET, and genetically engineered versions and/or variants thereof) and a β-glucosyltransferase (e.g., T4 BGT) may convert 5mC and 5hmC into products that cannot be deaminated, or are resistant to deamination, by a cytosine-deaminating enzyme (e.g., APOBEC). In a second reaction, a cytosine-deaminating enzyme (e.g., APOBEC) may deaminate unmodified (e.g., unmethylated) cytosines by converting them to uracils.

In another embodiment, TAPS can be used in enzymatic methylation sequencing operations. TAPS is a minimally-destructive conversion methylation sequencing method for converting cytosines to uracil in nucleic acid. This bisulfite-free method may allow minimal degradation of DNA, and may preserve the length of nucleic acid molecules while achieving conversion rates similar to sodium bisulfite sequencing. TAPS can result in higher sequencing quality scores for cytosines and guanine base pairs, and can provide a more even coverage of various genomic features, such as CpG islands.

In TAPS, a ten eleven translocation enzyme (e.g., TET1) is used to oxidize both 5mC and 5hmC to 5caC. Pyridine borane may be used to reduce 5caC to dihydrouracil, a uracil derivative that is converted to thymine after PCR. TAPS can be performed in two other ways: TAPSβ and chemical-assisted pyridine borane sequencing (CAPS). In TAPSβ, β-glucosyltransferase is used to label 5hmC with glucose to protect 5hmC from the oxidation and reduction reactions and allows for specific detection of 5mC. In CAPS, potassium perruthenate acts as the chemical replacement for Tet1 and specifically oxidizes 5hmC, thus allowing for direct detection.

### Hybrid Capture Panel

Hybrid capture panels were designed to cover the regions flanking the transcription start sites (+750 base pairs (window size: 1,500 base pairs)) of the protein-coding genes used for TSS-GAP featurization set forth in Tables 1-5. Biotinylated-DNA probes were generated against the genomic regions corresponding to each panel.

### Tss-GAP Sequencing

Libraries for the TSS-GAP assay were generated using a proprietary developed workflow that uses PBMC-derived MNAse-digested DNA or cfDNA.

The libraries were enriched for desired regions of interest using a hybrid capture protocol as described above utilizing the gene panels set forth in Tables 1-5. Biotinylated probes covering the gene panel were hybridized to the library DNA. Streptavidin coated beads were used to elute the probe-bound DNA molecules. The enriched libraries were then PCR amplified and subsequently sequenced using the Illumina Novaseq6000 to generate paired-end reads.

BCL files generated from sequencing were demultiplexed using bcl2fastq. FASTQ files were then trimmed using a proprietary workflow. Trimmed FASTQ files were used to generate BAM files using the human reference genome, hs38DH. Picard was used to mark duplicate reads in these BAM files.

### V-Plot Generation

V-plots were generated with fragment size information from deduplicated BAM files corresponding to regions of the DNA panel. Fragment start positions within the V-plots were defined relative to the Transcription Start Site (TSS) and assessed in 33 bp bins that extended outwards on both sides of the TSS. Fragment lengths were assessed in 16 bp bins up to a maximum size of 400 bp. The resulting 2-D heatmap displays the fragment count per genomic region bin along the x-axis and the fragment length bin along the y-axis.

### Tss-GAP Featurization

The protein-coding genes covered in the DNA panels were divided into training and test (holdout) gene sets for the purpose of TSS-GAP featurization. The training set consists of 595 “on” genes (Table 2) and 595 “off” genes (Table 1) with previously established typical expression patterns. V-plots were denoised using a Haar wavelet transform-based approach. Denoised v-plots corresponding to the training gene set were used to train a linear or non-linear model to classify a v-plot (one per gene per sample) as “on” or “off”. The corresponding logistic regression probabilities were used to generate gene activation (TSS-GAP) scores.

As shown in FIG. 3, gene activation was predicted from plasma cell-free DNA (cfDNA) or DNA isolated from PBMCs using both fragment length and fragment position for each of the protein-encoding genes listed in Tables 1-5. The TSS-GAP scores ranged from 0-1, where a score of 0 indicates the lowest possible activation score (non-expression) and a score of 1 indicates the highest possible activation score (expression) as described in more detail above.

While certain examples of methods and systems have been shown and disclosed herein, one of skill in the art will realize that these are provided by way of example only and not intended to be limiting within the specification. Numerous variations, changes, and substitutions will now occur to those skilled in the art without departing from the scope disclosed herein. Furthermore, it shall be understood that all aspects of the disclosed methods and systems are not limited to the specific depictions, configurations or relative proportions set forth herein which depend upon a variety of conditions and variables and the description is intended to include such alternatives, modifications, variations or equivalents.

1.-155. (canceled)

156. A method for preparing a methylation sequencing library for inferring gene expression, the method comprising:
(a) obtaining a biological sample from a subject;
(b) extracting cell-free deoxyribonucleic acid (cfDNA) from the biological sample, wherein the cfDNA comprises a plurality of cfDNA fragments;
(c) providing conditions capable of converting unmethylated cytosines to uracils in the cfDNA fragments to produce a plurality of converted cfDNA fragments;
(d) enriching the plurality of converted cfDNA fragments to produce enriched converted cfDNA fragment molecules, wherein the enriching comprises contacting the plurality of converted cfDNA fragments with a probe set comprising hybridization probes having sequence complementarity to at least two transcription start site (TSS) sequences of a plurality of TSS sequences selected from the genes listed in Tables 1-5;
(e) amplifying the enriched converted cfDNA fragment molecules to produce amplified enriched converted cfDNA fragments; and
(f) determining a nucleic acid sequence of a plurality of cfDNA sequencing fragments of the amplified enriched converted cfDNA fragments.

157. The method of claim 156, further comprising processing the plurality of cfDNA sequencing fragments, wherein the processing comprises calculating a gene expression score for one or more genes of a plurality of genes, wherein the gene expression score indicates a probability of expression or non-expression of the one or more genes of the plurality of genes.

158. The method of claim 156, wherein the plurality of TSS sequences are selected from the genes listed in Table 1.

159. The method of claim 156, wherein the plurality of TSS sequences are selected from the genes listed in Table 2.

160. The method of claim 156, wherein the plurality of TSS sequences are selected from the genes listed in Table 3.

161. The method of claim 156, wherein the plurality of TSS sequences are selected from the genes listed in Table 4.

162. The method of claim 156, wherein the plurality of TSS sequences are selected from the genes listed in Table 5.

163. The method of claim 156, wherein the biological sample comprises a blood sample or a cellular sample.

164. The method of claim 163, wherein the blood sample comprises a plasma sample, a serum sample, or a buffy coat sample.

165. The method of claim 163, wherein the cellular sample comprises a tissue sample, a biopsy sample, or a plurality of cells from a cell line.

166. The method of claim 156, wherein deoxyribonucleic acid (DNA) from the biological sample is treated with one or more nucleases prior to (b), wherein the one or more nucleases comprises micrococcal nuclease (MNase).

167. The method of claim 156, further comprising performing a sequencing assay on the plurality of cfDNA fragments.

168. The method of claim 167, wherein the sequencing assay comprises next generation sequencing (NGS), whole genome sequencing (WGS), bisulfite conversion, enzymatic conversion, or TET-assisted pyridine borane sequencing (TAPS) conversion.

169. The method of claim 156, further comprising determining fragmentation patterns in the plurality of cfDNA sequencing fragments.

170. The method of claim 169, further comprising using the fragmentation patterns to train a machine learning classifier capable of distinguishing between gene expression and gene non-expression.

171. The method of claim 157, wherein the gene expression score comprises a value of between 0 and 1, wherein a gene expression score of 0 corresponds to non-expression of the gene and a gene expression score of 1 corresponds to expression of the gene.

172. The method of claim 157, further comprising detecting a presence or an absence of a disease in the subject based at least in part on the gene expression score of the one or more genes.

173. The method of claim 172, further comprising administering a treatment to the subject based on detecting the presence of the disease in the subject.

174. The method of claim 156, further comprising administering a treatment to the subject based on determining a cancer subtype of the subject, or based on inferred gene expression patterns of a cancer in the subject.

175. A computer system for inferring gene expression, the system comprising:
(a) a non-transitory memory; and
(b) a processor in communication with the non-transitory memory, the processor configured to execute the following operations in order to effectuate a method comprising the operations of:
(i) obtaining a biological sample from a subject;
(ii) extracting cell-free DNA (cfDNA) from the biological sample, wherein the cfDNA comprises a plurality of cfDNA fragments;
(iii) performing a sequencing assay on the plurality of cfDNA fragments to generate a plurality of cfDNA sequencing fragments;
(iv) computer processing the plurality of cfDNA sequencing fragments; and
(v) calculating, based at least in part on the computer processing, a gene expression score for one or more genes of a plurality of genes, wherein the gene expression score indicates a probability of expression or non-expression of the one or more genes of the plurality of genes.
